(19) United States
(12) Patent Application Publication
(10) Pub. No.: US 2025/0345378 A1
(43) Pub. Date: Nov. 13, 2025

(54) PHAGE COMPOSITIONS FOR ESCHERICHIA COMPRISING CRISPR-CAS SYSTEMS AND METHODS OF USE THEREOF

(71) Applicant: Locus Biosciences, Inc., Morrisville, NC (US)

(72) Inventors: Hannah Hewitt Tuson, Morrisville, NC (US); Lana McMillan, Morrisville, NC (US); Brooke Sauder, Morrisville, NC (US)

(73) Assignee: Locus Biosciences, Inc., Morrisville, NC (US)

(21) Appl. No.: 18/862,452

(22) PCT Filed: May 3, 2023
(86) PCT No.: PCT/US2023/066566

§ 371 Date: Nov. 1, 2024

Related U.S. Application Data
(60) Provisional application No. 63/338,110, filed on May 4, 2022.

(51) Int. Cl. A61K 35/768; A61P 13/02; A61P 31/04; C07K 14/245; C12N 7/00; C12N 15/70

(52) U.S. Cl. CPC A61K 35/768; A61P 13/02; A61P 31/04; C07K 14/245; C12N 7/00; C12N 15/70; C12N 2795/10021; C12N 2795/10022; C12N 2795/10032; C12N 2795/10043; C12N 2795/10121; C12N 2795/10122; C12N 2795/10132

(57) ABSTRACT

Disclosed here are bacteriophage compositions for Escherichia comprising CRISPR-Cas systems and methods of use thereof.

### CROSS-REFERENCE

This application claims the benefit of U.S. Provisional Application No. 63/338,110 filed May 4, 2022, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on May 3, 2023, is named 53240-752_601_SL.xml and is 288 KB in size.

### SUMMARY

In certain aspects, described herein is a recombinant bacteriophage comprising a nucleic acid sequence encoding a Colicin 10, Colicin Ib, Colicin U, or Colicin K. In some embodiments, the Colicin 10 has at least 80% identity to SEQ ID NO 166. In some embodiments, the Colicin 10 is a homolog of the Colicin 10 having SEQ ID NO: 166. In some embodiments, the colicin comprises Colicin Ib. In some embodiments, the Colicin Ib has at least 80% identity to SEQ ID NO 165. In some embodiments, the Colicin Ib is a homolog of the Colicin Ib having SEQ ID NO: 165. In some embodiments, the colicin comprises Colicin 10. In some embodiments, the colicin comprises the Colicin U. In some embodiments, the Colicin U has at least 80% identity to SEQ ID NO 168. In some embodiments, the Colicin U is a homolog of the Colicin U having SEQ ID NO: 168. In some embodiments, the colicin comprises the Colicin K. In some embodiments, the Colicin K has at least 80% identity to SEQ ID NO 167. In some embodiments, the Colicin K is a homolog of the Colicin K having SEQ ID NO: 167. In some embodiments, the recombinant bacteriophage binds to and/or infects Escherichia. In some embodiments, the recombinant bacteriophage is a recombinant Tequatrovirus. In some embodiments, the recombinant Tequatrovirus is a recombinant p004k. In some embodiments, the recombinant bacteriophage has at least 80% sequence identity with p004k (ATCC Accession No. PTA-127149). In some embodiments, the recombinant Tequatrovirus is a recombinant p00ex. In some embodiments, the recombinant bacteriophage virus has at least 80% sequence identity with p00ex (ATCC Accession No. PTA-127145). In some embodiments, the recombinant bacteriophage is a recombinant Mosigvirus. In some embodiments, the recombinant Mosigvirus is a recombinant p00c0. In some embodiments, the recombinant bacteriophage virus has at least 80% sequence identity with p00c0 (ATCC Accession No. PTA-127143). In some embodiments, the recombinant bacteriophage is a recombinant Phapecoctavirus. In some embodiments, the recombinant Phapecoctavirus is a recombinant p00jc. In some embodiments, the recombinant bacteriophage virus has at least 80% sequence identity with p00jc (ATCC Accession No. PTA-127147). In some embodiments, the recombinant bacteriophage is a recombinant Myoviridae. In some embodiments, the recombinant Myoviridae is a recombinant p00ke. In some embodiments, the recombinant bacteriophage virus has at least 80% sequence identity with p00ke (ATCC Accession No. PTA-127148). In some embodiments, described herein is a composition comprising at least two of the recombinant bacteriophages described herein.

A recombinant Tequatrovirus bacteriophage comprising a nucleic acid sequence encoding Colicin U. In some embodiments, the nucleic acid encoding Colicin U is at least 90% identical to SEQ ID NO: 164, and the recombinant Tequatrovirus bacteriophage is at least 90% identical to p00ex (ATCC Accession No. PTA-127145). A recombinant Tequatrovirus bacteriophage comprising a nucleic acid sequence encoding Colicin K. In some embodiments, the nucleic acid encoding Colicin K is at least 90% identical to SEQ ID NO: 163, and the recombinant Tequatrovirus bacteriophage is at least 90% identical to p004k (ATCC Accession No. PTA-127149). A recombinant Phapecoctavirus bacteriophage comprising a nucleic acid sequence encoding Colicin 10. In some embodiments, the nucleic acid encoding Colicin 10 is at least 90% identical to SEQ ID NO: 162, and the recombinant Phapecoctavirus bacteriophage is at least 90% identical to p00jc (ATCC Accession No. PTA-127147). A recombinant Myoviridae bacteriophage comprising a nucleic acid sequence encoding Colicin 10. In some embodiments, the nucleic acid encoding Colicin 10 is at least 90% identical to SEQ ID NO: 161, and the recombinant Myoviridae bacteriophage is at least 90% identical to p00ke (ATCC Accession No. PTA-127148). In some embodiments, the nucleic acid sequence is operably linked to a promoter sequence. In some embodiments, the promoter sequence is at least about 80% to SEQ ID NO: 1-11, 19, 64 or 65. In some embodiments, the recombinant bacteriophage is an obligate lytic bacteriophage. In some embodiments, the recombinant bacteriophage is a temperate bacteriophage that is rendered lytic. In some embodiments, the temperate bacteriophage is rendered lytic by the removal, replacement, or inactivation of a lysogeny gene. In some embodiments, described herein is a composition comprising at least two of the recombinant bacteriophages described herein.

A recombinant bacteriophage comprising at least 80% sequence identity to p6921. A recombinant bacteriophage comprising at least 80% sequence identity to p6977. A recombinant bacteriophage comprising at least 80% sequence identity to p6984. A recombinant bacteriophage comprising at least 80% sequence identity to p00exe299. A recombinant bacteriophage comprising at least 80% sequence identity to p004ke127. A recombinant bacteriophage comprising at least 80% sequence identity to p00jce098. A recombinant bacteriophage comprising at least 80% sequence identity to p00exe296. A recombinant bacteriophage comprising at least 80% sequence identity to p00c0e103. In some embodiments, described herein is a composition comprising at least two of the recombinant bacteriophages described herein.

A composition comprising a first nucleic acid encoding a first Colicin, and a second nucleic acid encoding a second Colicin, wherein the first Colicin comprises Colicin Ib, and the second Colicin comprises Colicin 10, Colicin U, or Colicin K. In some embodiments, the second Colicin comprises Colicin 10. In some embodiments, the second Colicin comprises Colicin U. In some embodiments, the second Colicin comprises Colicin K.

A composition comprising a first nucleic acid encoding a first Colicin, and a second nucleic acid encoding a second Colicin, wherein the first Colicin comprises Colicin 10, and the second Colicin comprises Colicin U or Colicin K. In some embodiments, the second Colicin comprises Colicin U. In some embodiments, the second Colicin comprises Colicin K.

A composition comprising a first nucleic acid encoding a first Colicin, and a second nucleic acid encoding a second Colicin, wherein the first Colicin comprises Colicin U, and the second Colicin comprises Colicin K. A composition comprising a first nucleic acid encoding Colicin Ib, a second nucleic acid encoding Colicin 10, and a third nucleic acid encoding Colicin U. A composition comprising a first nucleic acid encoding Colicin Ib, a second nucleic acid encoding Colicin 10, and a third nucleic acid encoding Colicin K. A composition comprising a first nucleic acid encoding Colicin Ib, a second nucleic acid encoding Colicin U, and a third nucleic acid encoding Colicin K. A composition comprising a first nucleic acid encoding Colicin 10, a second nucleic acid encoding Colicin U, and third nucleic acid encoding Colicin K. A composition comprising a first nucleic acid encoding Colicin Ib, a second nucleic acid encoding Colicin 10, a third nucleic acid encoding Colicin U, and a fourth nucleic acid encoding Colicin K. In some embodiments, described herein is a method of killing bacteria comprising introducing into the bacteria genetic material from the recombinant bacteriophage or compositions described herein In some embodiments, described herein is a method of treating a disease in an individual in need thereof, the method comprising administering to the individual the recombinant bacteriophage or compositions described herein. In some embodiments, the disease is caused by a bacteria. In some embodiments, the bacteria is Escherichia (e.g., E. coli). A method of killing bacteria comprising contacting the bacteria with the composition described herein, thereby killing the bacteria.

In certain aspects described herein is a method of killing a population of bacteria, the method comprising contacting the population of bacteria with one or more Colicins, wherein the one or more Colicins comprises Colicin Ib, Colicin 10, Colicin U, or Colicin K, or a combination of two or more thereof. In some embodiments, the one or more Colicins comprises Colicin Ib. In some embodiments, the one or more Colicins comprises Colicin 10. In some embodiments, the one or more Colicins comprises Colicin U. In some embodiments, the one or more Colicins comprises Colicin K.

In certain aspects, described herein is a method of reducing resistance to a bacteriophage in a population of target bacteria, comprising administering the bacteriophage to the population of target bacteria, wherein the bacteriophage comprises comprising a nucleic acid sequence encoding a Colicin Ib, Colicin 10, Colicin U, or Colicin K; wherein the population of target bacteria administered the bacteriophage comprising a nucleic acid sequence has a lower concentration of target bacteriophage at 24 hours following administration when compared to a second population of target bacteriophage administered a wildtype bacteriophage. In some embodiments, described herein is a method of treating a urinary tract infection in a subject in need thereof, the method comprising administering the recombinant bacteriophage or composition described herein to a subject in need thereof; wherein the recurrent urinary tract infection comprises a Escherichia coli infection, wherein the first bacteriophage and the second bacteriophage target E. coli. In some embodiments, described herein is a method of reducing the levels of Escherichia coli in a subject, the method comprising administering the recombinant bacteriophage or composition described herein to a subject in need thereof, wherein the Escherichia coli concentration is or has been measured in a blood or urine sample of the patient. In some embodiments, described herein is the use of the recombinant bacteriophage or composition described herein for the treatment of a disease in a subject in need thereof. In some embodiments, described herein is a manufacture of a medicament the recombinant bacteriophage or composition described herein for use in treatment of a disease in a subject in need thereof. In some embodiments, described herein is a kit comprising the recombinant bacteriophage or composition described herein.

### BRIEF DESCRIPTION OF THE DRAWINGS

The novel features of the disclosures are set forth with particularity in the appended claims. A better understanding of the features and advantages of the present disclosures will be obtained by reference to the following detailed description that sets forth illustrative embodiments, in which the principles of the disclosures are utilized, and the accompanying drawings of which:

FIG. 1A depicts the sequence and arrangement of the crArray containing the SC2 spacer (SEQ ID NO: 42, with linker SEQ ID NO: 55). FIG. 1B depicts the array sequence for bacteriophages p004Ke009, p00c0e030, and p00exe014 (labeled PAIC array 2, SEQ ID NO: 43). FIG. 1C depicts the pJC_Cfp1 array sequence (array: SEQ ID NO: 44, sequence shown: SEQ ID NO: 56).

FIG. 2 is a schematic representation of the genome of wild type bacteriophage p004k and its engineered variant p004ke007. The bar below the genome axis indicates the region of the genome that was removed and replaced. The schematic below the bacteriophage genome illustrates the DNA that was used to replace WT bacteriophage genes in the deleted region.

FIGS. 3A-3D illustrates the efficacy of the crArray/Cas system insert in E. coli. FIG. 3A depicts the growth on an agar plate of p004kwt (wild type) and p004ke007 (SC2+Cas system) mixed with three strains of E. coli (b3402, b3418, or b4098). FIGS. 3B-3D are a quantification of the optical density of the cell growth in b3402, b3418, and b4098, respectively.

FIG. 4 depicts the growth on an agar plate of p00EX.015 and p00EXe014.008 (3 spacer full construct) mixed with two strains of E. coli (b2185 or b3911).

FIG. 5 shows an exemplary CRISPR-Cas system for testing selected constructs.

FIG. 6 illustrates the plasmid based experimental set up to test crRNA array in targeting bacteria.

FIG. 7 shows data demonstrating that the spacers have high coverage and efficacy.

FIG. 8 shows data from individual PAIC spacer kill test.

FIG. 9A shows crRNA alone causes no statistical reduction in CFU formation. FIGS. 9B-9C shows data indicating selected spacers do not activate endogenous EcIF or EcIE systems. FIG. 9B shows ECIF crosstalk of the identified spacers. FIG. 9C shows ECIE activity of PAIC spacers.

FIG. 10 shows a mutational model for predicting number of targeting sites for mitigating mutational escape.

FIG. 11 shows plasmid-based PAIC array kill data.

FIG. 12A-12C shows host range of CK618 and antibiotics on 88-isolate panel. FIG. 12A, Host range on all 88 strains. FIG. 12B, Host range on 21 MDR strains. FIG. 12C, Host range on 31 strains with any beta-lactam resistance excluding cephems. Single treatment is treatment with either CK618 or antibiotic. Antibiotic+CK618 is the combination of antibiotic and CK618.

FIG. 13A-13C shows host range of CK618 and antibiotics on 304-isolate panel. FIG. 13A, Host range on all 304 strains. FIG. 13B, Host range on 93 MDR strains. FIG. 13C, Host range on 91 strains with any beta-lactam resistance excluding cephems. Single treatment is treatment with either CK618 or antibiotic. Antibiotic+CK618 is the combination of antibiotic and CK618.

FIG. 14A shows the schematic design of in vivo testing of the engineered bacteriophage efficacy using CK570 bacteriophage cocktail. FIG. 14B shows the schematic design of in vivo testing of the engineered bacteriophage efficacy using CK618 bacteriophage cocktail.

FIGS. 15A-15C show data demonstrating reduction in bacterial burden in kidney (FIG. 15A), bladder (FIG. 15B) and urine (FIG. 15C) following CK570 administration in mouse UTI model.

FIGS. 16A-16E show data demonstrating detectable levels of bacteriophage in kidney (FIG. 16A), bladder (FIG. 16B), urine (FIG. 16C), blood (FIG. 16D) and spleen (FIG. 16E) following CK570 administration.

FIGS. 17A-17B show data demonstrating reduction in bacterial burden in kidney (FIG. 17A), and bladder (FIG. 17B) following CK618 administration in mouse UTI model.

FIGS. 18A-18C show data demonstrating biodistribution of intravenous CK618 administered either as single dose or as five twice daily doses, with or without an initial intraurethral dose of CK618, in kidney (FIG. 18A), bladder (FIG. 18B), and blood plasma (FIG. 18C).

FIG. 19 shows graphical representations of AUC (area under the curve) ratio and time to OD (optical density).

FIG. 20 depicts the maximum log CFU reduction observed on treatment with cocktail CK570.

FIG. 21 depicts the correlation of liquid host range values with CFU reduction results.

FIGS. 22A-22C depicts Host Range of LBP-EC01 v.2 and Antibiotics on 88-Isolate Panel. FIG. 22A depicts the host range on all 88 strains. FIG. 22B depicts the host range on 21 MDR strains. FIG. 22C depicts the host range on 31 strains with any beta-lactam resistance excluding cephems. Single treatment is treatment with either LBP-EC01 v.2 or antibiotic. Antibiotic+LBP-EC01 v.2 is the combination of antibiotic and LBP-EC01 v.2.

FIG. 23A-23C depicts Host Range of LBP-EC01 v.2 and Antibiotics on 304-Isolate Panel. FIG. 23A depicts the host range on all 304 isolates. FIG. 23B depicts the host range on 93 MDR strains. FIG. 23C depicts the host range on 91 strains with any beta-lactam resistance excluding cephems. Single treatment is treatment with either LBP-EC01 v.2 or antibiotic. Antibiotic+LBP-EC01 v.2 is the combination of antibiotic and LBP-EC01 v.2.

FIG. 24 depicts a study schematic for a UTI model.

FIG. 25 depicts the efficacy of intravenous ck570 in combination with TMP/SMX (bactrim), dosed twice daily over five days, in reducing bacterial burden in a mouse UTI model. ANOVA=analysis of variance; CFU=colony-forming unit; h=hour; IU=intraurethral; IV=intravenous; UTI=urinary tract infection. Data are displayed as mean±standard error of the mean. Statistical significance was determined by one-way ANOVA with Tukey's post-test. *p<0.05, **p<0.01, ***p<0.001, ****p<0.0001.

FIG. 26 depicts similar detectable levels of bacteriophage after administration of CK570 as monotherapy or in combination with TMP/SMX (Bactrim). Data are displayed as mean±standard error of the mean. Statistical significance was determined by one-way ANOVA with Tukey's post-test. *p<0.05, **p<0.01, ***p<0.001, ****p<0.0001.

FIG. 27 depicts a study schematic for a UTI model.

FIG. 28 depicts the efficacy of intravenous CK618, dosed twice daily for a total of five doses, with or without an initial intraurethral dose of CK618 in reducing bacterial burden in a mouse UTI model. Data are displayed as mean±standard error of the mean. Statistical significance was determined by one-way ANOVA with Tukey's post-test. *p<0.05, **p<0.01, ***p<0.001, ****p<0.0001.

FIG. 29 depicts the biodistribution of intravenous CK618, administered as either a single dose or as five twice-daily doses, with or without an initial intraurethral dose of CK618. Data are displayed as mean±standard error of the mean. Statistical significance was determined by one-way ANOVA with Tukey's post-test. *p<0.05, **p<0.01, ***p<0.001, ****p<0.0001.

FIG. 30 depicts a study schematic for a UTI model.

FIG. 31 depicts an overlaid mean plot and scatter plot of urine concentrations of LBP-EC01 (PFU/mL) for Day 1 on linear and semi-logarithmic scale (PK Population). The LLOQ value (5 PFU/mL) was used for concentration results of 0 PFU/mL for the semi-logarithmic scale. The PK Population was defined as all patients who received at least 1 dose of LBP-EC01 and had at least 1 measurable post-dose concentration of LBP-EC01.

FIG. 32 depicts Mean (±SD) Plot of Log Change in E. coli Burden by Visit (Maximum Approach1; Modified ITT Population2). Maximum approach involved taking the highest CFU/mL for any individual strain of E. coli present in a sample at a given timepoint. The Modified ITT Population was defined as all patients in the ITT Population who did not have 1 of the following: antibiotic use during the study, low baseline E. coli colony count (below 1×103 CFU/mL), early withdrawal from IMP dosing, possessed mixed flora or no growth at baseline or up to Day 2, IMP bacteriophage detected in a patient who received the placebo, or possessed an SAE.

FIG. 33 depicts a case study: subject 106-012 demonstrates proof of mechanism bacteriophage amplification with corresponding PD response.

FIG. 34 depicts the study design of the clinical trial.

FIG. 35 depicts colicin activity against an E. coli clinical validation panel.

FIG. 36 depicts the varying activity of different colicins against an E. coli clinical validation panel.

FIG. 37 depicts the improvement in bacterial suppression of 22 E. coli clinical isolates following treatment with p004ke124 (Colicin Ib), p004ke125 (Colicin K), or CK1325 (p004ke124 [Colicin Ib]+p004ke125 [Colicin K]) compared to treatment with p004k (wild-type) 24 hours following phage administration.

FIG. 38 depicts the bacterial titer of E. coli strain b5421 after treatment with bacteriophage p004k WT, p004k124 (Colicin Ib), p004k125 (Colicin K), or CK1325 (p004ke124 [Colicin Ib]+p004ke125 [Colicin K]) over time.

FIG. 39 depicts the improvement in bacterial suppression of 19 E. coli clinical isolates following treatment with p00jce098 (Colicin 10), p00jce172 (Colicin K), or p00jce096 (Colicin Ib) compared to treatment with p00jc (wild-type) 24 hours following administration.

FIG. 40 depicts the improvement in bacterial suppression of 7 E. coli clinical isolates following treatment with p00jce098 (Colicin 10), p00jce175 (Colicin U), or p00jce172 (Colicin K) compared to treatment with p00jc (wild-type) 24 hours following administration.

FIG. 41 depicts the improvement in bacterial suppression of 21 E. coli clinical isolates following treatment with p00exe296 (Colicin Ib), p00exe297 (Colicin K), or p00exe291 (Colicin 10) compared to treatment with p00ex (wild-type) 24 hours after administration.

FIG. 42 depicts the improvement in bacterial suppression of 11 E. coli clinical isolates following treatment with p00exe296 (Colicin Ib), p00exe299 (Colicin U), p00exe297 (Colicin K), or p00exe300 (Colicin K) compared to treatment with p00ex (wild-type) 24 hours after administration.

FIG. 43 depicts the percentage of replicates at limit of detection (LOD) 24 hours after treating 77 E. coli clinical isolates with bacteriophage cocktails CK000618, CK001369, CK001360, CK001391, CK001352, CK001372, or CK001390.

FIG. 44 depicts the percentage of replicates at limit of detection (LOD) 24 hours after treating 57 E. coli clinical isolates with bacteriophage cocktails CK000618, CK001360, CK001391, CK001352, or CK001372.

FIG. 45 depicts improvement in killing of 8 E. coli clinical isolates when treated with a phage cocktail containing colicin-engineered phages (CK001391 or CK001372 compared to a control (cells only) or to treatment with a phage cocktail that does not contain colicin-engineered phages (CK000618, CK001360, or CK001352) over different CFU/mL input concentrations.

FIG. 46 depicts the final bacterial titer of E. coli strain b3414 24 hours after treatment of various input bacterial titers with bacteriophage cocktails CK000618, CK001360, CK001391, CK001352, or CK001372.

FIG. 47 depicts the bacterial titer of E. coli strain b3414 4 hours after treatment with various MOIs of bacteriophage cocktails CK000618, CK001360, CK001391, CK001352, or CK001372.

FIG. 48 depicts the final bacterial titer of E. coli strain b3970 24 hours after treatment of various input bacterial titers with bacteriophage cocktails CK000618, CK001360, CK001391, CK001352, or CK001372.

FIG. 49 depicts the final bacterial titer of E. coli strain b3414 24 hours after treatment with various MOIs of bacteriophage cocktails CK000618, CK001360, CK001391, CK001352, or CK001372.

FIG. 50 depicts the percentage of all 215 E. coli strains in a clinical isolate panel that are at LOD in a CFU reduction assay compared to the subset of 80 Bactrim-resistant (BactrimR) and 104 multidrug-resistant (MDR) strains 24 hours after administration of CK000618, CK001360, CK001391, CK001352, or CK001372.

FIG. 51 depicts a study schematic for a neutropenic thigh model.

FIG. 52 depicts the concentration of E. coli strain b3370 in the mouse thigh 4 or 7 hours after administration of Colicin 10, Colicin U, or a combination therapy of Colicin 10+Colicin U.

FIG. 53A depicts the concentration (CFU/mL) of E. coli strain b3370 after administration of Col10 at 0.0295 nM-29.5 nM, measured 24 hours following administration. FIG. 53B depicts the concentration (CFU/mL) of E. coli strain b3370 after administration of Col10 at 0.0295 nM-29.5 nM, measured 24 hours following administration. FIG. 53C depicts the concentration (CFU/mL) of E. coli strain b3411 after administration of Col10 at 0.0295 nM-29.5 nM, measured 24 hours following administration. FIG. 53D depicts the concentration (CFU/mL) of E. coli strain b3411 after administration of Col10 at 0.0295 nM-29.5 nM, measured 24 hours following administration.

FIG. 54 depicts the concentration of E. coli strain b3370 in the mouse thigh 4 or 7 hours after administration of increasing concentrations of Colicin 10.

### DETAILED DESCRIPTION

Disclosed herein, are compositions and methods for killing an Escherichia species. In some embodiments, the compositions comprise a lytic bacteriophage comprising a colicin, a CRISPR-Cas system, or a combination thereof. In some embodiments, the compositions comprise a cocktail comprising at least two, three, four, five, six, seven, or eight bacteriophage described herein.

Additional bacteriophage embodiments disclosed herein include a bacteriophage modified to replace bacteriophage DNA with a nucleic acid sequence encoding a CRISPR system comprising: a CRISPR array comprising a spacer sequence complementary to a target nucleotide sequence in a target bacteria, and a sequence encoding a CRISPR nuclease.

Also disclosed herein, in certain embodiments, are pharmaceutical compositions comprising the bacteriophages disclosed herein.

Further disclosed herein, in certain embodiments, are methods of killing a Escherichia species comprising introducing into the Escherichia species a bacteriophage described herein. Further disclosed herein, in certain embodiments, are methods of treating a disease in an individual in need thereof, the method comprising administering to the individual a bacteriophage described herein. The bacteriophage in some embodiments is a wild-type bacteriophage. The bacteriophage in some embodiments is an engineered bacteriophage. The bacteriophage in some embodiments comprises one or more bacteriophage of Table 1A, or at least 80% sequence identity to one or more bacteriophage of Table 1A. The bacteriophage may comprise two or more different bacteriophage in a cocktail, e.g., CK618.

The present disclosure further provides nucleic acid sequences, e.g., for incorporation into a bacteriophage. In some embodiments, the nucleic acid sequence has at least 80% identity to any one of SEQ ID NOS: 38-44. Some such nucleic acid sequences comprise a spacer and/or a repeat sequence, wherein the spacer sequence if present is complementary to a target bacteria sequence.

### Escherichia

In some embodiments, the bacterium comprises one or more species of Escherichia. In some embodiments, the bacterium comprises one or more strains of Escherichia. In some embodiments, the target bacterium is Escherichia coli.

In some embodiments, the E. coli causes an infection or disease. In some embodiments, the infection or disease is acute or chronic. In some embodiments, the infection or disease is localized or systemic. In some embodiments, infection or disease is idiopathic. In some embodiments, the infection or disease is acquired through means including, but not limited to, respiratory inhalation, ingestion, skin and wound infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, infections of indwelling medical devices such as joint prostheses, dental implants, catheters and cardiac implants, sexual contact, and/or hospital-acquired and ventilator-associated bacterial pneumonias. In some embodiments, the E. coli causes urinary tract infection. In some embodiments, the E. coli causes and/or exacerbates an inflammatory disease. In some embodiments, the E. coli causes and/or exacerbates an autoimmune disease. In some embodiments, the E. coli causes and/or exacerbates inflammatory bowel disease (IBD). In some embodiments, the E. coli causes inflammatory bowel disease (IBD). In some embodiments, the E. coli causes and/or exacerbates psoriasis. In some embodiments, the E. coli causes and/or exacerbates psoriatic arthritis (PA). In some embodiments, the E. coli causes and/or exacerbates rheumatoid arthritis (RA). In some embodiments, the E. coli causes and/or exacerbates systemic lupus erythematosus (SLE). In some embodiments, the E. coli causes and/or exacerbates multiple sclerosis (MS). In some embodiments, the E. coli causes and/or exacerbates Graves' disease. In some embodiments, the E. coli causes and/or exacerbates Hashimoto's thyroiditis. In some embodiments, the E. coli causes and/or exacerbates Myasthenia gravis. In some embodiments, the E. coli causes and/or exacerbates vasculitis. In some embodiments, the E. coli causes and/or exacerbates cancer. In some embodiments, the E. coli causes and/or exacerbates cancer progression. In some embodiments, the E. coli causes and/or exacerbates cancer metastasis. In some embodiments, the E. coli causes and/or exacerbates resistance to cancer therapy. In some embodiments, the therapy used to address cancer includes, but is not limited to, chemotherapy, immunotherapy, hormone therapy, targeted drug therapy, and/or radiation therapy. In some embodiments, the cancer develops in organs including, but not limited to the, anus, bladder, blood and blood components, bone, bone marrow, brain, breast, cervix uteri, colon and rectum, esophagus, kidney, larynx, lymphatic system, muscle (i.e., soft tissue), oral cavity and pharynx, ovary, pancreas, prostate, skin, small intestine, stomach, testis, thyroid, uterus, and/or vulva. In some embodiments, the E. coli causes and/or exacerbates disorders of the central nervous system (CNS). In some embodiments, the E. coli causes and/or exacerbates attention deficit/hyperactivity disorder (ADHD). In some embodiments, the E. coli causes and/or exacerbates autism. In some embodiments, the E. coli causes and/or exacerbates bipolar disorder. In some embodiments, the E. coli causes and/or exacerbates major depressive disorder. In some embodiments, the E. coli causes and/or exacerbates epilepsy. In some embodiments, the E. coli causes and/or exacerbates neurodegenerative disorders including, but not limited to, Alzheimer's disease, Huntington's disease, and/or Parkinson's disease.

In some embodiments, one or more bacteriophage are administered to a patient with cystic fibrosis or cystic fibrosis-associated bronchiectasis. In some embodiments, a combination of two or more bacteriophage are administered to a patient with cystic fibrosis or cystic fibrosis-associated bronchiectasis. In some embodiments, administration of the bacteriophage to a patient with cystic fibrosis or cystic fibrosis-associated bronchiectasis results in a reduction in bacterial load in the patient. In some embodiments, the reduction in bacterial load results in a clinical improvement in the patient with cystic fibrosis or cystic fibrosis-associated bronchiectasis.

In some embodiments, one or more bacteriophage are administered to a patient with non-cystic fibrosis bronchiectasis. In some embodiments, a combination of two or more bacteriophage are administered to a patient with non-cystic fibrosis bronchiectasis. In some embodiments, administration of the bacteriophage to a patient with non-cystic fibrosis bronchiectasis results in a reduction in bacterial load in the patient. In some embodiments, the reduction in bacterial load results in a clinical improvement in the patient with non-cystic fibrosis bronchiectasis.

### Colicins

Bacteriocins are antibacterial proteins produced by bacteria to kill other bacteria. In some embodiments, they target specific or related species. Bacteriocins produced by E. coli may be referred to as “colicins”. Without being limited by theory, colicins function to kill other bacteria, such as E. coli, through various mechanisms, including targeting enzymatic activity, and forming pores in target bacteria. In some embodiments, the colicins described herein function through pore formation. In some embodiments, the colicin comprises Colicin Ib, Colicin 10, Colicin U, Colicin K, Colicin Ia, Colicin E1, Colicin D, Colicin E2, Colicin E3, or Colicin B.

In certain aspects, described herein are compositions comprising a first nucleic acid encoding a first colicin and a second nucleic acid encoding a second colicin. In some embodiments, the first colicin comprises Colicin Ib and the second colicin comprises Colicin 10, Colicin U, or Colicin K. In some embodiments, the second colicin comprises Colicin 10. In some embodiments, the second colicin comprises Colicin U. In some embodiments, the second colicin comprises Colicin K. In some embodiments, the first Colicin comprises Colicin 10, and the second Colicin comprises Colicin U or Colicin K. In some embodiments, the second Colicin comprises Colicin U. In some embodiments, the second Colicin comprises Colicin K. In some embodiments, the first Colicin comprises Colicin U, and the second Colicin comprises Colicin K.

In some embodiments, described herein is a composition comprising a first nucleic acid encoding Colicin Ib, a second nucleic acid encoding Colicin 10, and a third nucleic acid encoding Colicin U. In some embodiments, described herein is a composition comprising a first nucleic acid encoding Colicin Ib, a second nucleic acid encoding Colicin 10, and a third nucleic acid encoding Colicin K. In some embodiments, described herein is a composition comprising a first nucleic acid encoding Colicin Ib, a second nucleic acid encoding Colicin U, and a third nucleic acid encoding Colicin K. In some embodiments, described herein is a composition comprising a first nucleic acid encoding Colicin 10, a second nucleic acid encoding Colicin U, and third nucleic acid encoding Colicin K. In some embodiments, described herein is a composition comprising a first nucleic acid encoding Colicin Ib, a second nucleic acid encoding Colicin 10, a third nucleic acid encoding Colicin U, and a fourth nucleic acid encoding Colicin K.

In some embodiments, the bacteriophage described herein comprises a nucleic acid sequence encoding a colicin. In some embodiments, the colicin comprises Colicin Ib, Colicin 10, Colicin U, or Colicin K, or homologs thereof.

In some embodiments, the bacteriophage comprises a nucleic acid sequence encoding Colicin Ib (ColIb), or homologs thereof. In some embodiments, the colicin comprises Colicin Ib (e.g. a sequence at least 80% identical to SEQ ID NO 165). In some embodiments, the colicin comprises a homolog of Colicin Ib. In some embodiments, the colicin comprises at least about 70%, 80%, 85%, 90%, 91%, 92%, 93%, 94%, 95%, 96%, 97%, 98%, 99%, or 100% with SEQ ID NO 165. In some embodiments, the colicin is encoded by a sequence comprising at least about 70%, 80%, 85%, 90%, 91%, 92%, 93%, 94%, 95%, 96%, 97%, 98%, 99%, or 100% sequence identity with SEQ ID NO: 161.

In some embodiments, the bacteriophage comprises a nucleic acid sequence encoding Colicin 10 (Col10), or homologs thereof. In some embodiments, the colicin comprises Colicin 10 (e.g. a sequence at least 80% identical to SEQ ID NO 166). In some embodiments, the colicin comprises a homolog of Colicin 10. In some embodiments, the colicin comprises at least about 70%, 80%, 85%, 90%, 91%, 92%, 93%, 94%, 95%, 96%, 97%, 98%, 99%, or 100% with SEQ ID NO 166. In some embodiments, the colicin is encoded by a sequence comprising at least about 70%, 80%, 85%, 90%, 91%, 92%, 93%, 94%, 95%, 96%, 97%, 98%, 99%, or 100% sequence identity with SEQ ID NO: 162.

In some embodiments, the bacteriophage comprises a nucleic acid sequence encoding Colicin K (ColK), or homologs thereof. In some embodiments, the colicin comprises Colicin K (e.g. a sequence at least 80% identical to SEQ ID NO 167). In some embodiments, the colicin comprises a homolog of Colicin K. In some embodiments, the colicin comprises at least about 70%, 80%, 85%, 90%, 91%, 92%, 93%, 94%, 95%, 96%, 97%, 98%, 99%, or 100% with SEQ ID NO 167. In some embodiments, the colicin is encoded by a sequence comprising at least about 70%, 80%, 85%, 90%, 91%, 92%, 93%, 94%, 95%, 96%, 97%, 98%, 99%, or 100% sequence identity with SEQ ID NO: 163.

In some embodiments, the bacteriophage comprises a nucleic acid sequence encoding Colicin U (ColU), or homologs thereof. In some embodiments, the colicin comprises Colicin U (e.g. a sequence at least 80% identical to SEQ ID NO 168). In some embodiments, the colicin comprises a homolog of Colicin U. In some embodiments, the colicin comprises at least about 70%, 80%, 85%, 90%, 91%, 92%, 93%, 94%, 95%, 96%, 97%, 98%, 99%, or 100% with SEQ ID NO 168. In some embodiments, the colicin is encoded by a sequence comprising at least about 70%, 80%, 85%, 90%, 91%, 92%, 93%, 94%, 95%, 96%, 97%, 98%, 99%, or 100% sequence identity with SEQ ID NO: 164.

In some embodiments, the bacteriophage comprises a nucleic acid sequence encoding Colicin Ia (ColIa), or homologs thereof. In some embodiments, the colicin comprises Colicin Ia (e.g. a sequence at least 80% identical to SEQ ID NO 177). In some embodiments, the colicin comprises a homolog of Colicin Ia. In some embodiments, the colicin comprises at least about 70%, 80%, 85%, 90%, 91%, 92%, 93%, 94%, 95%, 96%, 97%, 98%, 99%, or 100% with SEQ ID NO 177. In some embodiments, the colicin is encoded by a sequence comprising at least about 70%, 80%, 85%, 90%, 91%, 92%, 93%, 94%, 95%, 96%, 97%, 98%, 99%, or 100% sequence identity with SEQ ID NO: 171.

In some embodiments, the bacteriophage comprises a nucleic acid sequence encoding Colicin E1 (ColE1), or homologs thereof. In some embodiments, the colicin comprises Colicin E1 (e.g. a sequence at least 80% identical to SEQ ID NO 178). In some embodiments, the colicin comprises a homolog of Colicin E1. In some embodiments, the colicin comprises at least about 70%, 80%, 85%, 90%, 91%, 92%, 93%, 94%, 95%, 96%, 97%, 98%, 99%, or 100% with SEQ ID NO 178. In some embodiments, the colicin is encoded by a sequence comprising at least about 70%, 80%, 85%, 90%, 91%, 92%, 93%, 94%, 95%, 96%, 97%, 98%, 99%, or 100% sequence identity with SEQ ID NO: 172.

In some embodiments, the bacteriophage comprises a nucleic acid sequence encoding Colicin D (ColD), or homologs thereof. In some embodiments, the colicin comprises Colicin D (e.g. a sequence at least 80% identical to SEQ ID NO 179). In some embodiments, the colicin comprises a homolog of Colicin D. In some embodiments, the colicin comprises at least about 70%, 80%, 85%, 90%, 91%, 92%, 93%, 94%, 95%, 96%, 97%, 98%, 99%, or 100% with SEQ ID NO 179. In some embodiments, the colicin is encoded by a sequence comprising at least about 70%, 80%, 85%, 90%, 91%, 92%, 93%, 94%, 95%, 96%, 97%, 98%, 99%, or 100% sequence identity with SEQ ID NO: 173.

In some embodiments, the bacteriophage comprises a nucleic acid sequence encoding Colicin E2 (ColE2), or homologs thereof. In some embodiments, the colicin comprises Colicin U (e.g. a sequence at least 80% identical to SEQ ID NO 180). In some embodiments, the colicin comprises a homolog of Colicin E2. In some embodiments, the colicin comprises at least about 70%, 80%, 85%, 90%, 91%, 92%, 93%, 94%, 95%, 96%, 97%, 98%, 99%, or 100% with SEQ ID NO 180. In some embodiments, the colicin is encoded by a sequence comprising at least about 70%, 80%, 85%, 90%, 91%, 92%, 93%, 94%, 95%, 96%, 97%, 98%, 99%, or 100% sequence identity with SEQ ID NO: 174.

In some embodiments, the bacteriophage comprises a nucleic acid sequence encoding Colicin E3 (ColE3), or homologs thereof. In some embodiments, the colicin comprises Colicin E3 (e.g. a sequence at least 80% identical to SEQ ID NO 181). In some embodiments, the colicin comprises a homolog of Colicin E3. In some embodiments, the colicin comprises at least about 70%, 80%, 85%, 90%, 91%, 92%, 93%, 94%, 95%, 96%, 97%, 98%, 99%, or 100% with SEQ ID NO 181. In some embodiments, the colicin is encoded by a sequence comprising at least about 70%, 80%, 85%, 90%, 91%, 92%, 93%, 94%, 95%, 96%, 97%, 98%, 99%, or 100% sequence identity with SEQ ID NO: 175.

In some embodiments, the bacteriophage comprises a nucleic acid sequence encoding Colicin B (ColB), or homologs thereof. In some embodiments, the colicin comprises Colicin B (e.g. a sequence at least 80% identical to SEQ ID NO 182). In some embodiments, the colicin comprises a homolog of Colicin B. In some embodiments, the colicin comprises at least about 70%, 80%, 85%, 90%, 91%, 92%, 93%, 94%, 95%, 96%, 97%, 98%, 99%, or 100% with SEQ ID NO 182. In some embodiments, the colicin is encoded by a sequence comprising at least about 70%, 80%, 85%, 90%, 91%, 92%, 93%, 94%, 95%, 96%, 97%, 98%, 99%, or 100% sequence identity with SEQ ID NO: 176.

### CRISPR/CAS Systems

CRISPR-Cas systems are naturally adaptive immune systems found in bacteria and archaea. The CRISPR system is a nuclease system involved in defense against invading bacteriophages and plasmids that provides a form of acquired immunity. There is a diversity of CRISPR-Cas systems based on the set of cas genes and their phylogenetic relationship. There are at least six different types (I through VI) where Type I represents over 50% of all identified systems in both bacteria and archaea. In some embodiments, a Type I, Type II, Type III, Type IV, Type V, or Type VI CRISPR-Cas system is used herein.

Type I systems are divided into seven subtypes including: Type I-A, Type I-B, Type I-C, Type I-D, Type I-E, Type I-F, and Type I-U. Type I CRISPR-Cas systems include a multi-subunit complex called Cascade (for complex associated with antiviral defense), Cas3 (a protein with nuclease, helicase, and exonuclease activity that is responsible for degradation of the target DNA), and CRISPR array encoding crRNA (stabilizes Cascade complex and directs Cascade and Cas3 to DNA target). Cascade forms a complex with the crRNA, and the protein-RNA pair recognizes its genomic target by complementary base pairing between the 5′ end of the crRNA sequence and a predefined protospacer. This complex is directed to homologous loci of pathogen DNA via regions encoded within the crRNA and protospacer-adjacent motifs (PAMs) within the pathogen genome. Base pairing occurs between the crRNA and the target DNA sequence leading to a conformational change. In the Type I-E system, the PAM is recognized by the CasA protein within Cascade, which then unwinds the flanking DNA to evaluate the extent of base pairing between the target and the spacer portion of the crRNA. Sufficient recognition leads Cascade to recruit and activate Cas3. Cas3 then nicks the non-target strand and begins degrading the strand in a 3′-to-5′ direction.

In the Type I-C system, the proteins Cas5, Cas8c, and Cas7 form the Cascade effector complex. Cas5 processes the pre-crRNA (which can take the form of a multi-spacer array, or a single spacer between two repeats) to produce individual crRNA(s) made up of a hairpin structure formed from the remaining repeat sequence and a linear spacer. The effector complex then binds to the processed crRNA and scans DNA to identify PAM sites. In the Type I-C system, the PAM is recognized by the Cas8c protein, which then acts to unwind the DNA duplex. If the sequence 3′ of the PAM matches the crRNA spacer that is bound to effector complex, a conformational change in the complex occurs and Cas3 is recruited to the site. Cas3 then nicks the non-target strand and begins degrading the DNA.

In some embodiments, the CRISPR-Cas system is endogenous to the Escherichia species. In some embodiments, the CRISPR-Cas system is exogenous to the Escherichia species. In some embodiments, the CRISPR-Cas system is a Type I CRISPR-Cas system. In some embodiments, the CRISPR-Cas system is a Type I-A CRISPR-Cas system. In some embodiments, the CRISPR-Cas system is a Type I-B CRISPR-Cas system. In some embodiments, the CRISPR-Cas system is a Type I-C CRISPR-Cas system. In some embodiments, the CRISPR-Cas system is a Type I-D CRISPR-Cas system. In some embodiments, the CRISPR-Cas system is a Type I-E CRISPR-Cas system. In some embodiments, the CRISPR-Cas system is a Type I-F CRISPR-Cas system. In some embodiments, the CRISPR-Cas system is a Type I-U CRISPR-Cas system.

In some embodiments, the CRISPR-Cas system is a Type II CRISPR-Cas system. In some embodiments, the CRISPR-Cas system is a Type III CRISPR-Cas system. In some embodiments, the CRISPR-Cas system is a Type IV CRISPR-Cas system. In some embodiments, the CRISPR-Cas system is a Type VI CRISPR-Cas system.

In some embodiments, processing of a CRISPR-array disclosed herein includes, but is not limited to, the following processes: 1) transcription of the nucleic acid encoding a pre-crRNA; 2) recognition of the pre-crRNA by Cascade and/or specific members of Cascade, such as Cas6, and 3) processing of the pre-crRNA by Cascade or members of Cascade, such as Cas6, into mature crRNAs. In some embodiments, the mode of action for a Type I CRISPR system includes, but is not limited to, the following processes: 4) mature crRNA complexation with Cascade; 5) target recognition by the complexed mature crRNA/Cascade complex; and 6) nuclease activity at the target leading to DNA degradation.

### CRISPR Bacteriophages

Disclosed herein, in certain embodiments, are bacteriophage compositions comprising CRISPR-Cas systems and methods of use thereof.

Bacteriophages or “phages” represent a group of bacterial viruses and are engineered or sourced from environmental sources. Individual bacteriophage host ranges are usually narrow, meaning, bacteriophages are highly specific to one strain or few strains of a bacterial species and this specificity makes them unique in their antibacterial action. Bacteriophages are bacterial viruses that rely on the host's cellular machinery to replicate. Bacteriophages are generally classified as virulent or temperate bacteriophages depending on their lifestyle. Virulent bacteriophages, also known as lytic bacteriophages, can only undergo lytic replication. Lytic bacteriophages infect a host cell, undergo numerous rounds of replication, and trigger cell lysis to release newly made bacteriophage particles. In some embodiments, the lytic bacteriophages disclosed herein retain their replicative ability. In some embodiments, the lytic bacteriophages disclosed herein retain their ability to trigger cell lysis. In some embodiments, the lytic bacteriophages disclosed herein retain both they replicative ability and the ability to trigger cell lysis. In some embodiments, the bacteriophages disclosed herein comprise a CRISPR array. In some embodiments, the CRISPR array does not affect the bacteriophages ability to replicate and/or trigger cell lysis. Temperate or lysogenic bacteriophages can undergo lysogeny in which the bacteriophage stops replicating and stably resides within the host cell, either integrating into the bacterial genome or being maintained as an extrachromosomal plasmid. Temperate bacteriophages can also undergo lytic replication similar to their lytic bacteriophage counterparts. Whether a temperate bacteriophage replicates lytically or undergoes lysogeny upon infection depends on a variety of factors including growth conditions and the physiological state of the cell. A bacterial cell that has a lysogenic bacteriophage integrated into its genome is referred to as a lysogenic bacterium or lysogen. Exposure to adverse conditions may trigger reactivation of the lysogenic bacteriophage, termination of the lysogenic state and resumption of lytic replication by the bacteriophage. This process is called induction. Adverse conditions which favor the termination of the lysogenic state include desiccation, exposure to UV or ionizing radiation, and exposure to mutagenic chemicals. This leads to the expression of the bacteriophage genes, reversal of the integration process, and lytic multiplication. In some embodiments, the temperate bacteriophages disclosed herein are rendered lytic. The term “lysogeny gene” refers to any gene whose gene product promotes lysogeny of a temperate bacteriophage. Lysogeny genes can directly promote, as in the case of integrase proteins that facilitate integration of the bacteriophage into the host genome. Lysogeny genes can also indirectly promote lysogeny as in the case of CI transcriptional regulators which prevent transcription of genes required for lytic replication and thus favor maintenance of lysogeny.

Bacteriophages package and deliver synthetic DNA using three general approaches. Under the first approach, the synthetic DNA is recombined into the bacteriophage genome in a targeted manner, which usually involves a selectable marker. Under the second approach, restriction sites within the bacteriophage are used to introduce synthetic DNA in-vitro. Under the third approach, a plasmid generally encoding the bacteriophage packaging sites and lytic origin of replication is packaged as part of the assembly of the bacteriophage particle. The resulting plasmids have been coined “phagemids.”

Phages are limited to a given bacterial strain for evolutionary reasons. In some cases, injecting their genetic material into an incompatible strain is counterproductive. Bacteriophages have therefore evolved to specifically infect a limited cross-section of bacterial strains. However, some bacteriophages have been discovered that inject their genetic material into a wide range of bacteria. The classic example is the P1 bacteriophage, which has been shown to inject DNA in a range of gram-negative bacteria.

Disclosed herein, in some embodiments, are bacteriophages comprising a first nucleic acid sequence encoding a first spacer sequence or a crRNA transcribed therefrom, wherein the first spacer sequence is complementary to a target nucleotide sequence from a target gene in a Escherichia species. In some embodiments, the bacteriophage comprises a first nucleic acid sequence encoding a first spacer sequence or a crRNA transcribed therefrom, wherein the first spacer sequence is complementary to a target nucleotide sequence from a target gene in a Escherichia species, provided that the bacteriophage is rendered lytic. In some embodiments, the bacteriophage is a temperate bacteriophage. In some embodiments, the bacteriophage is rendered lytic by removal, replacement, or inactivation of a lysogenic gene. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the bacteriophage is rendered lytic by the removal of a regulatory element of a lysogeny gene. In some embodiments, the bacteriophage is rendered lytic by the removal of a promoter of a lysogeny gene. In some embodiments, the bacteriophage is rendered lytic by the removal of a functional element of a lysogeny gene. In some embodiments, the lysogenic gene is an activator gene. In some embodiments, the lysogenic gene is cII gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, the bacteriophage is rendered lytic via a second CRISPR array comprising a second spacer directed to a lysogenic gene. In some embodiments, the bacteriophage is rendered lytic by the insertion of one or more lytic genes. In some embodiments, the bacteriophage is rendered lytic by the insertion of one or more genes that contribute to the induction of a lytic cycle. In some embodiments, the bacteriophage is rendered lytic by altering the expression of one or more genes that contribute to the induction of a lytic cycle. In some embodiments, the bacteriophage phenotypically changes from a lysogenic bacteriophage to a lytic bacteriophage. In some embodiments, the phenotypic change is via a self-targeting CRISPR-Cas system to render a bacteriophage lytic since it is incapable of lysogeny. In some embodiments, the self-targeting CRISPR-Cas comprises a self-targeting crRNA from the prophage genome and kills lysogens. In some embodiments, the bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI)). In some embodiments, the bacteriophage that is rendered lytic is prevented from reverting to lysogenic state. In some embodiments, the bacteriophage that is rendered lytic is prevented from reverting back to lysogenic state by way of introducing an additional CRIPSR array. In some embodiments, the bacteriophage does not confer any new properties onto the Escherichia species beyond cellular death cause by lytic activity of the bacteriophage and/or the activity of the CRISPR array. Further disclosed herein, in some embodiments, are temperate bacteriophages comprising a first nucleic acid sequence encoding a first spacer sequence or a crRNA transcribed therefrom, wherein the first spacer sequence is complementary to a target nucleotide sequence from a target gene in a Escherichia species, provided the bacteriophage is rendered lytic. In some embodiments, the bacteriophage infects multiple bacterial strains. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential gene that is needed for survival of the Escherichia species. In some embodiments, the gene is Tsf acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK. In some embodiments, the target nucleotide sequence is in a non-essential gene. Non-limiting example non-essential genes include ppSa (e.g., SC2), raiA (e.g., SC6), and intergenic conserved repeat (e.g., SC6). In some embodiments, the target nucleotide sequence is a noncoding sequence. In some embodiments, the noncoding sequence is an intergenic sequence. In some embodiments, the spacer sequence is complementary to a target nucleotide sequence of a highly conserved sequence in a Escherichia species. In some embodiments, the spacer sequence is complementary to a target nucleotide sequence of a sequence present in the Escherichia species. In some embodiments, the spacer sequence is complementary to a target nucleotide sequence that comprises all or a part of a promoter sequence of the essential gene. In some embodiments, the first nucleic acid sequence comprises a first CRISPR array comprising at least one repeat sequence. In some embodiments, the at least one repeat sequence is operably linked to the first spacer sequence at either its 5′ end or its 3′ end.

In some embodiments, the bacteriophage or phagemid DNA is from a lysogenic or temperate bacteriophage. In some embodiments, the bacteriophages or phagemids include but are not limited to P1 bacteriophage, a M13 bacteriophage, a λ bacteriophage, a T4 bacteriophage, a ϕC2 bacteriophage, a ϕCD27 bacteriophage, a ϕNM1 bacteriophage, Bc431 v3 bacteriophage, ϕ10 bacteriophage, ϕ25 bacteriophage, ϕ151 bacteriophage, A511-like bacteriophages, B054, 0176-like bacteriophages, or Campylobacter bacteriophages (such as NCTC 12676 and NCTC 12677). In some embodiments, the bacteriophage includes, but is not limited to p004ke007, p004Ke005 (ATCC Accession No. PTA-127150), p004K (ATCC Accession No. PTA-127149), p00c0e030 (Accession No. PTA-127144), p00c0e103, p00c0 (ATCC Accession No. PTA-127143), p00exe014 (ATCC Accession No PTA-127146), p00ex (ATCC Accession No. PTA-127145), p00jc (ATCC Accession No. PTA-127147), p00ke (ATCC Accession No. PTA-127148), p5516 (ATCC Accession No. PTA-127151), p0031, p0078, p00c8, or p0033L.

In some embodiments, a plurality of bacteriophages are used together. In some embodiments, the plurality of bacteriophages used together targets the same or different bacteria within a sample or subject.

In some embodiments, bacteriophages of interest are obtained from environmental sources or commercial research vendors. In some embodiments, obtained bacteriophages are screened for lytic activity against a library of bacteria and their associated strains. In some embodiments, the bacteriophages are screened against a library of bacteria and their associated strains for their ability to generate primary resistance in the screened bacteria.

In some embodiments, the nucleic acid is inserted into the bacteriophage genome. In some embodiments, the nucleic acid comprises a crArray, a Cas system, or a combination thereof. In some embodiments, the nucleic acid is inserted into the bacteriophage genome at a transcription terminator site at the end of an operon of interest. In some embodiments, the nucleic acid is inserted into the bacteriophage genome as a replacement for one or more removed non-essential genes. In some embodiments, the nucleic acid is inserted into the bacteriophage genome as a replacement for one or more removed lysogenic genes. In some embodiments, the replacement of non-essential and/or lysogenic genes with the nucleic acid enhances the lytic activity of the bacteriophage. In some embodiments, the replacement of non-essential and/or lysogenic genes with the nucleic acid renders a lysogenic bacteriophage lytic.

In some embodiments, the nucleic acid is introduced into the bacteriophage genome at a first location while one or more non-essential and/or lysogenic genes are separately removed and/or inactivated from the bacteriophage genome at a separate location. In some embodiments, the removal of one or more non-essential and/or lysogenic genes renders a lysogenic bacteriophage into a lytic bacteriophage. Similarly, in some embodiments, one or more lytic genes are introduced into the bacteriophage so as to render a non-lytic, lysogenic bacteriophage into a lytic bacteriophage.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

In some embodiments, the non-essential gene to be removed and/or replaced from the bacteriophage is a gene that is non-essential for the survival of the bacteriophage. In some embodiments, the non-essential gene to be removed and/or replaced from the bacteriophage is a gene that is non-essential for the induction and/or maintenance of lytic cycle.

Disclosed herein, in certain embodiments, are bacteriophages comprising a complete exogenous CRISPR system. In some embodiments, the CRISPR-Cas system is Type I CRISPR-Cas system, Type II CRISPR-Cas system, Type III CRISPR-Cas system, Type IV CRISPR-Cas system, Type V CRISPR-Cas system, or Type VI CRISPR-Cas system. Disclosed herein, in certain embodiments, are bacteriophages comprising a nucleic acid sequence encoding a Type I CRISPR-Cas system comprising: (a) a CRISPR array; (b) a Cascade polypeptide; and (c) a Cas3 polypeptide. Example Type I component sequences are provided as SEQ ID NOS: 47-54. Disclosed herein, in certain embodiments, are bacteriophages comprising a nucleic acid sequence encoding a Type V CRISPR-Cpf1 (Cas12a) system comprising a CRISPR array associated with a Cpf1 nuclease polypeptide. An example Cpf1 sequence is provided as SEQ ID NO: 46. In some embodiments, a CRISPR system comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOS: 46-54. In some embodiments, a CRISPR system comprises any one of SEQ ID NOS: 46-54. For instance, the CRISPR system comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 46; a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 47; a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 48; a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 49; a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 50; a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 51; a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 52; a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 53; or a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 54; or any combination thereof.

In some embodiments, the bacteriophage is p004ke007. In some embodiments, the bacteriophage is p004Ke005. In some embodiments, the bacteriophage is an engineered p004K. In some embodiments, the bacteriophage is an engineered p00c0. In some embodiments, the bacteriophage is an engineered p00ex. In some embodiments, the bacteriophage is p00jc. In some embodiments, the bacteriophage is p00ke. In some embodiments, the bacteriophage is p5516. In some embodiments, the bacteriophage is p004ke009. In some embodiments, the bacteriophage is p00c0e030. In some embodiments, the bacteriophage is p00c0e103. In some embodiments, the bacteriophage is p00exe014. In some embodiments, the bacteriophage is p0031. In some embodiments, the bacteriophage is p0078. In some embodiments, the bacteriophage is p00c8. In some embodiments, the bacteriophage is p0033L. In some embodiments, the bacteriophage comprises a bacteriophage listed in Table 1A. In some embodiments, the bacteriophage comprises a CRISPR-Cas system.

In some embodiments, a plurality of bacteriophages are used together. In some embodiments, the plurality of bacteriophages used together targets the same or different bacteria within a sample or subject. In some embodiments, a cocktail comprising a plurality of bacteriophages is used together. In some embodiments, the cocktail comprises at least 2, 3, 4, 5, 6, 7, 8, 9, 10, or more bacteriophages selected from Table 1A. In some embodiments, the cocktail comprises 2 bacteriophages selected from Table 1A. In some embodiments, the cocktail comprises 3 bacteriophages selected from Table 1A. In some embodiments, the cocktail comprises 3 bacteriophages selected from Table 1A. In some embodiments, the cocktail comprises 4 bacteriophages selected from Table 1A. In some embodiments, the cocktail comprises 5 bacteriophages selected from Table 1A. In some embodiments, the cocktail comprises 6 bacteriophages selected from Table 1A. In some embodiments, at least one bacteriophage in the cocktail comprises a CRISPR array (e.g., comprising one or more components of Tables 2-4). In some embodiments, at least 2, 3, 4, 5, 6, 7, 8, 9, 10, or more bacteriophages present in the cocktail comprise a CRISPR array. In some embodiments, at least one bacteriophage in the cocktail comprises a nucleic acid sequence encoding a Cascade polypeptide. In some embodiments, at least 2, 3, 4, 5, 6, 7, 8, 9, 10, or more bacteriophages present in the cocktail comprise a nucleic acid sequence encoding a Cascade polypeptide. In some embodiments, at least one bacteriophage in the cocktail comprises a nucleic acid sequence encoding a Cas3 polypeptide. In some embodiments, at least 2, 3, 4, 5, 6, 7, 8, 9, 10, or more bacteriophages present in the cocktail comprise a nucleic acid sequence encoding a Cas3 polypeptide. In some embodiments, the cocktail comprises p004ke009, p00c0e030, p00exe014, wherein each bacteriophage comprises a Type I CRISPR-Cas system. In some embodiments, the cocktail further comprises p5516. In some embodiments, the cocktail further comprises p00jc. In some embodiments, the cocktail further comprises p00ke. In some embodiments, the cocktail further comprises p0031. In some embodiments, the cocktail further comprises p0078. In some embodiments, the cocktail further comprises p00c8. In some embodiments, the cocktail further comprises p0033L.

### CRISPR Array

In some embodiments, the CRISPR array (crArray) comprises a spacer sequence and at least one repeat sequence. In some embodiments, the CRISPR array encodes a processed, mature crRNA. In some embodiments, the mature crRNA is introduced into a bacteriophage or an Escherichia species. In some embodiments, an endogenous or exogenous Cas6 processes the CRISPR array into mature crRNA. In some embodiments, an exogenous Cas6 is introduced into the bacteriophage. In some embodiments, the bacteriophage comprises an exogenous Cas6. In some embodiments, an exogenous Cas6 is introduced into a Escherichia species.

In some embodiments, the CRISPR array comprises a spacer sequence. In some embodiments, the CRISPR array further comprises at least one repeat sequence. In some embodiments, the at least one repeat sequence is operably linked to the spacer sequence at either its 5′ end or its 3′ end. In some embodiments, the CRISPR array is of any length and comprises any number of spacer nucleotide sequences alternating with repeat nucleotide sequences necessary to achieve the desired level of killing of a Escherichia species by targeting one or more essential genes. In some embodiments, the CRISPR array comprises, consists essentially of, or consists of 1 to about 100 spacer nucleotide sequences, each linked on its 5′ end and its 3′ end to a repeat nucleotide sequence. In some embodiments, the CRISPR array comprises, consists essentially of, or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 38. In some embodiments, the CRISPR array comprises SEQ ID NO: 38. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a spacer from Table 3 (e.g., 1, 2, or 3 spacers from Table 3). In some embodiments, the CRISPR array comprises a spacer from Table 3. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a repeat from Table 4 (e.g., 1, 2, or 3 spacers from Table 3). In some embodiments, the CRISPR array comprises a repeat from Table 4. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a promoter from Table 1A. In some embodiments, the CRISPR array comprises a promoter from Table 1A.

In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 39. In some embodiments, the CRISPR array comprises SEQ ID NO: 39. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a spacer from Table 3 (e.g., 1, 2, or 3 spacers from Table 3). In some embodiments, the CRISPR array comprises a spacer from Table 3. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a repeat from Table 4 (e.g., 1, 2, or 3 spacers from Table 3). In some embodiments, the CRISPR array comprises a repeat from Table 4. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a promoter from Table 1A. In some embodiments, the CRISPR array comprises a promoter from Table 1A.

In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 40. In some embodiments, the CRISPR array comprises SEQ ID NO: 40. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a spacer from Table 3 (e.g., 1, 2, or 3 spacers from Table 3). In some embodiments, the CRISPR array comprises a spacer from Table 3. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a repeat from Table 4 (e.g., 1, 2, or 3 spacers from Table 3). In some embodiments, the CRISPR array comprises a repeat from Table 4. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a promoter from Table 1A. In some embodiments, the CRISPR array comprises a promoter from Table 1A.

In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 41. In some embodiments, the CRISPR array comprises SEQ ID NO: 41. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a spacer from Table 3 (e.g., 1, 2, or 3 spacers from Table 3). In some embodiments, the CRISPR array comprises a spacer from Table 3. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a repeat from Table 4 (e.g., 1, 2, or 3 spacers from Table 3). In some embodiments, the CRISPR array comprises a repeat from Table 4. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a promoter from Table 1A. In some embodiments, the CRISPR array comprises a promoter from Table 1A.

In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 42. In some embodiments, the CRISPR array comprises SEQ ID NO: 42. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a spacer from Table 3 (e.g., 1, 2, or 3 spacers from Table 3). In some embodiments, the CRISPR array comprises a spacer from Table 3. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a repeat from Table 4 (e.g., 1, 2, or 3 spacers from Table 3). In some embodiments, the CRISPR array comprises a repeat from Table 4. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a promoter from Table 1A. In some embodiments, the CRISPR array comprises a promoter from Table 1A.

In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 43. In some embodiments, the CRISPR array comprises SEQ ID NO: 43. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a spacer from Table 3 (e.g., 1, 2, or 3 spacers from Table 3). In some embodiments, the CRISPR array comprises a spacer from Table 3. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a repeat from Table 4 (e.g., 1, 2, or 3 spacers from Table 3). In some embodiments, the CRISPR array comprises a repeat from Table 4. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a promoter from Table 1A. In some embodiments, the CRISPR array comprises a promoter from Table 1A.

In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 44. In some embodiments, the CRISPR array comprises SEQ ID NO: 44. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a spacer from Table 3 (e.g., 1, 2, or 3 spacers from Table 3). In some embodiments, the CRISPR array comprises a spacer from Table 3. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a repeat from Table 4 (e.g., 1, 2, or 3 spacers from Table 3). In some embodiments, the CRISPR array comprises a repeat from Table 4. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a promoter from Table 1A. In some embodiments, the CRISPR array comprises a promoter from Table 1A.

### Spacer Sequence

In some embodiments, the spacer sequence is complementary to a target nucleotide sequence in a Escherichia species. In some embodiments, the target nucleotide sequence is a coding region. In some embodiments, the coding region is an essential gene. In some embodiments, the coding region is a nonessential gene. In some embodiments, the target nucleotide sequence is a noncoding sequence. In some embodiments, the noncoding sequence is an intergenic sequence. In some embodiments, the spacer sequence is complementary to a target nucleotide sequence of a highly conserved sequence in a Escherichia species. In some embodiments, the spacer sequence is complementary to a target nucleotide sequence of a sequence present in the Escherichia species. In some embodiments, the spacer sequence is complementary to a target nucleotide sequence that comprises all or a part of a promoter sequence of the essential gene. In some embodiments, the spacer sequence comprises one, two, three, four, or five mismatches as compared to the target nucleotide sequence. In some embodiments, the mismatches are contiguous. In some embodiments, the mismatches are noncontiguous. In some embodiments, the spacer sequence has 70% complementarity to a target nucleotide sequence. In some embodiments, the spacer sequence has 80% complementarity to a target nucleotide sequence. In some embodiments, the spacer sequence is 85%, 90%, 95%, 96%, 97%, 98%, 99% or 100% complementarity to a target nucleotide sequence. In some embodiments, the spacer sequence has 100% complementarity to the target nucleotide sequence. In some embodiments, the spacer sequence has complete complementarity or substantial complementarity over a region of a target nucleotide sequence that are at least about 8 nucleotides to about 150 nucleotides in length. In some embodiments, a spacer sequence has complete complementarity or substantial complementarity over a region of a target nucleotide sequence that is at least about 20 nucleotides to about 100 nucleotides in length. In some embodiments, the 5′region of the spacer sequence is 100% complementary to a target nucleotide sequence while the 3′ region of the spacer is substantially complementary to the target nucleotide sequence and therefore the overall complementarity of the spacer sequence to the target nucleotide sequence is less than 100%. For example, in some embodiments, the first 7, 8, 9, 10, 11, 12, 13, 14, 15, 16 nucleotides in the 3′ region of a 20 nucleotide spacer sequence (seed region) is 100% complementary to the target nucleotide sequence, while the remaining nucleotides in the 5′ region of the spacer sequence are substantially complementary (e.g., at least about 70% complementary) to the target nucleotide sequence. In some embodiments, the first 7 to 12 nucleotides of the 3′ end of the spacer sequence is 100% complementary to the target nucleotide sequence, while the remaining nucleotides in the 5′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target nucleotide sequence. In some embodiments, the first 7 to 10 nucleotides in the 3′ end of the spacer sequence is 75%-99% complementary to the target nucleotide sequence, while the remaining nucleotides in the 5′ region of the spacer sequence are at least about 50% to about 99% complementary to the target nucleotide sequence. In some embodiments, the first 7 to 10 nucleotides in the 3′ end of the spacer sequence is 100% complementary to the target nucleotide sequence, while the remaining nucleotides in the 5′ region of the spacer sequence are substantially complementary (e.g., at least about 70% complementary) to the target nucleotide sequence. In some embodiments, the first 10 nucleotides (within the seed region) of the spacer sequence is 100% complementary to the target nucleotide sequence, while the remaining nucleotides in the 5′ region of the spacer sequence are substantially complementary (e.g., at least about 70% complementary) to the target nucleotide sequence. In some embodiment, the 5′ region of a spacer sequence (e.g., the first 8 nucleotides at the 5′ end, the first 10 nucleotides at the 5′ end, the first 15 nucleotides at the 5′ end, the first 20 nucleotides at the 5′ end) have about 75% complementarity or more (75% to about 100% complementarity) to the target nucleotide sequence, while the remainder of the spacer sequence have about 50% or more complementarity to the target nucleotide sequence. In some embodiments, the first 8 nucleotides at the 5′ end of the spacer sequence have 100% complementarity to the target nucleotide sequence or have one or two mutations and therefore is about 88% complementary or about 75% complementary to the target nucleotide sequence, respectively, while the remainder of the spacer nucleotide sequence is at least about 50% or more complementary to the target nucleotide sequence.

In some embodiments, the spacer sequence is about 15 nucleotides to about 150 nucleotides in length. In some embodiments, the spacer nucleotide sequence is about 15 nucleotides to about 100 nucleotides in length (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides or more). In some embodiments, the spacer nucleotide sequence is a length of about 8 to about 150 nucleotides, about 8 to about 100 nucleotides, about 8 to about 50 nucleotides, about 8 to about 40 nucleotides, about 8 to about 30 nucleotides, about 8 to about 25 nucleotides, about 8 to about 20 nucleotides, about 10 to about 150 nucleotides, about 10 to about 100 nucleotides, about 10 to about 80 nucleotides, about 10 to about 50 nucleotides, about 10 to about 40, about 10 to about 30, about 10 to about 25, about 10 to about 20, about 15 to about 150, about 15 to about 100, about 15 to about 50, about 15 to about 40, about 15 to about 30, about 20 to about 150 nucleotides, about 20 to about 100 nucleotides, about 20 to about 80 nucleotides, about 20 to about 50 nucleotides, about 20 to about 40, about 20 to about 30, about 20 to about 25, at least about 8, at least about 10, at least about 15, at least about 20, at least about 25, at least about 30, at least about 32, at least about 35, at least about 40, at least about 44, at least about 50, at least about 60, at least about 70, at least about 80, at least about 90, at least about 100, at least about 110, at least about 120, at least about 130, at least about 140, at least about 150 nucleotides in length, or more, and any value or range therein. In some embodiments, the P. aeruginosa Type I-C Cas system has a spacer length of about 30 to 39 nucleotides, about 31 to about 38 nucleotides, about 32 to about 37 nucleotides, about 33 to about 36 nucleotides, about 34 to about 35 nucleotides, or about 35 nucleotides In some embodiments, the P. aeruginosa Type I-C Cas system has a spacer length of about 34 nucleotides. In some embodiments, the P. aeruginosa Type I-C Cas system has a spacer length of at least about 10, at least about 15, at least about 20, at least about 21, at least about 22, at least about 23, at least about 24, at least about 25, at least about 26, at least about 27, at least about 29, at least about 29, at least about 30, at least about 31, at least about 32, at least about 33, at least about 34, at least about, at least about 35, at least about 36, at least about 37, at least about 38, at least about 39, at least about 20, at least about 41, at least about 42, at least about 43, at least about 44, at least about 45, or more than about 45 nucleotides.

In some embodiments, the spacer sequence comprises at least or about 70%, 80%, 85%, 90%, 91%, 92%, 93%, 94%, 95%, 96%, 97%, 98%, 99%, or 100% sequence identity to SEQ ID NO: 12 or 20-37. In some instances, the spacer sequence comprises at least or about 95% sequence identity to SEQ ID NO: 12 or 20-37. In some instances, the spacer sequence comprises at least or about 97% sequence identity to SEQ ID NO: 12 or 20-37. In some instances, the spacer sequence comprises at least or about 99% sequence identity to SEQ ID NO: 12 or 20-37. In some instances, the spacer sequence comprises 100% sequence identity to SEQ ID NO: 12 or 20-37. In some instances, the spacer sequence comprises at least a portion having at least or about 3, 4, 5, 6, 7, 8, 9, 10, 12, 14, 16, 17, 18, 19, 20, 21, 22, 23, 24, 25, 26, 27, 28, 29, 30, 31, 32, 33, 34, or more than 34 nucleotides of SEQ ID NO: 12 or 20-37.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

The term “homology,” “sequence identity” or “similarity” between two proteins is determined by comparing the amino acid sequence and its conserved amino acid substitutes of one protein sequence to the second protein sequence. Similarity may be determined by procedures which are well-known in the art, for example, a BLAST program (Basic Local Alignment Search Tool at the National Center for Biological Information).

In some embodiments, the identity of two or more spacer sequences of the CRISPR array is the same. In some embodiments, the identity of two or more spacer sequences of the CRISPR array is different. In some embodiments, the identity of two or more spacer sequences of the CRISPR array is different but are complementary to one or more target nucleotide sequences. In some embodiments, the identity of two or more spacer sequences of the CRISPR array is different and are complementary to one or more target nucleotide sequences that are overlapping sequences. In some embodiments, the identity of two or more spacer sequences of the CRISPR array is different and are complementary to one or more target nucleotide sequences that are not overlapping sequences. In some embodiments, the target nucleotide sequence is about 10 to about 40 consecutive nucleotides in length located immediately adjacent to a PAM sequence (PAM sequence located immediately 3′ of the target region) in the genome of the organism. In some embodiments, a target nucleotide sequence is located adjacent to or flanked by a PAM (protospacer adjacent motif).

The PAM sequence is found in the target gene next to the region to which a spacer sequence binds as a result of being complementary to that region and identifies the point at which base pairing with the spacer nucleotide sequence begins. The exact PAM sequence that is required varies between each different CRISPR-Cas system and is identified through established bioinformatics and experimental procedures. Non-limiting examples of PAMs include CCA, CCT, CCG, TTC, AAG, AGG, ATG, GAG, and/or CC. For Type I systems, the PAM is located immediately 5′ to the sequence that matches the spacer, and thus is 3′ to the sequence that base pairs with the spacer nucleotide sequence, and is directly recognized by Cascade. Once a protospacer is recognized, Cascade generally recruits the endonuclease Cas3, which cleaves and degrades the target DNA. For Type II systems, the PAM is required for a Cas9/sgRNA to form an R-loop to interrogate a specific DNA sequence through Watson-Crick pairing of its guide RNA with the genome. The PAM specificity is a function of the DNA-binding specificity of the Cas9 protein (e.g., a -protospacer adjacent motif recognition domain at the C-terminus of Cas9).

In some embodiments, the target nucleotide sequence in the bacterium to be killed is any essential target nucleotide sequence of interest. In some embodiments, the target nucleotide sequence is a non-essential sequence. In some embodiments, a target nucleotide sequence comprises, consists essentially of or consist of all or a part of a nucleotide sequence encoding a promoter, or a complement thereof, of the essential gene. In some embodiments, the spacer nucleotide sequence is complementary to a promoter, or a part thereof, of the essential gene. In some embodiments, the target nucleotide sequence comprises all or a part of a nucleotide sequence located on a coding or a non-coding strand of the essential gene. In some embodiments, the target nucleotide sequence comprises all or a part of a nucleotide sequence located on a coding of a transcribed region of the essential gene.

In some embodiments, the essential gene is any gene of an organism that is critical for its survival. However, being essential is highly dependent on the circumstances in which an organism lives. For instance, a gene required to digest starch is only essential if starch is the only source of energy. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential gene that is needed for survival of the Escherichia species. In some embodiments, the essential gene is Tsf acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK. In some embodiments, the target nucleotide sequence comprises a non-essential gene or a portion thereof. In some embodiments, a non-essential gene is any gene of an organism that is not critical for survival. However, being non-essential is highly dependent on the circumstances in which an organism lives. Non-limiting example non-essential genes include ppSa (e.g., SC2), raiA (e.g., SC6), and intergenic conserved repeat (e.g., SC6).

In some embodiments, non-limiting examples of the target nucleotide sequence of interest includes a target nucleotide sequence encoding a transcriptional regulator, a translational regulator, a polymerase gene, a metabolic enzyme, a transporter, an RNase, a protease, a DNA replication enzyme, a DNA modifying or degrading enzyme, a regulatory RNA, a transfer RNA, or a ribosomal RNA. In some embodiments, the target nucleotide sequence is from a gene involved in cell-division, cell structure, metabolism, motility, pathogenicity, virulence, or antibiotic resistance. In some embodiments, the target nucleotide sequence is from a hypothetical gene whose function is not yet characterized. Thus, for example, these genes are any genes from any bacterium.

The appropriate spacer sequences for a full-construct bacteriophage may be identified by locating a search set of representative genomes, searching the genomes with relevant parameters, and determining the quality of a spacer for use in a CRISPR engineered bacteriophage.

First, a suitable search set of representative genomes is located and acquired for the organism/species/target of interest. The set of representative genomes may be found in a variety of databases, including without limitations the NCBI genbank or the PATRIC database. NCBI genbank is one of the largest databases available and contains a mixture of reference and submitted genomes for nearly every organism sequenced to date. Specifically, for pathogenic prokaryotes, the PATRIC (Pathosystems Resource Integration Center) database provides an additional comprehensive resource of genomes and provides a focus on clinically relevant strains and genomes relevant to a drug product. Both of the above databases allow for bulk downloading of genomes via FTP (File Transfer Protocol) servers, enabling rapid and programmatic dataset acquisition.

Next, the genomes are searched with relevant parameters to locate suitable spacer sequences. Genomes may be read from start to end, in both the forward and reverse complement orientations, to locate contiguous stretches of DNA that contain a PAM (Protospacer Adjacent Motif) site. The spacer sequence will be the N-length DNA sequence 3′ or 5′ adjacent to the PAM site (depending on the CRISPR system type), where N is specific to the Cas system of interest and is generally known ahead of time. Characterizing the PAM sequence and spacer sequences may be performed during the discovery and initial research of a Cas system. Every observed PAM-adjacent spacer may be saved to a file and/or database for downstream use. The exact PAM sequence that is required varies between each different CRISPR-Cas system and is identified through established bioinformatics and experimental procedures.

Next, the quality of a spacer for use in a CRISPR engineered bacteriophage is determined. Each observed spacer may be evaluated to determine how many of the evaluated genomes they are present in. The observed spacers may be evaluated to see how many times they may occur in each given genome. Spacers that occur in more than one location per genome may be advantageous because the Cas system may not be able to recognize the target site if a mutation occurs, and each additional “backup” site increases the likelihood that a suitable, non-mutated target location will be present. The observed spacers may be evaluated to determine whether they occur in functionally annotated regions of the genome. If such information is available, the functional annotations may be further evaluated to determine whether those regions of the genome are “essential” for the survival and function of the organism. By focusing on spacers that occur in all, or nearly all, evaluated genomes of interest (>=99%), the spacer selection may be broadly applicable to many targeted genomes. Provided a large selection pool of conserved spacers exists, preference may be given to spacers that occur in regions of the genome that have known function, with higher preference given if those genomic regions are “essential” for survival and occur more than 1 time per genome.

The spacer sequences for a full construct bacteriophage, in some embodiments, are validated. In some embodiments, a first step comprises identifying a plasmid that replicates in the organism, species, or target of interest. In some embodiments, the plasmid has a selectable marker. In some embodiments, the selectable marker is an antibiotic-resistance gene. In some embodiments, an expression cassette includes a nucleotide sequence for a selectable marker. In some embodiments, the selectable marker is adenine deaminase (ada), blasticidin S deaminases (Bsr, BSD), bleomycin-binding protein (Ble), Neomycin phosphotransferase (neo), histidinol dehydrogenase (hisD), glutamine synthetase (GS), dihydrofolate reductase (dhfr), cytosine deaminase (codA), puromycin N-acetyltransferase (Pac), or hygromycin B phosphotransferase (Hph), ampicillin, chloramphenicol, kanamycin, tetracycline, polymyxin B, erythromycin, carbenicillin, streptomycin, spectinomycin, puromycin N-acetyltransferase (Pac), or zeocin (Sh bla). In some embodiments, the selectable marker is a gene involved in thymidylate synthase, thymidine kinase, dihydrofolate reductase, or glutamine synthetase. In some embodiments, the selectable marker is a gene encoding a fluorescent protein.

In some embodiments, a second step comprises inserting the genes encoding the Cas system into the plasmid such that they will be expressed in the organism, species, or target of interest. In some embodiments, a promoter is provided upstream of the Cas system. In some embodiments, the promoter is recognized by the organism, species, or target of interest to drive the expression of the Cas system. Exemplary promoters include, but are not limited to, L-arabinose inducible (araBAD, P_{BAD}) promoter, any lac promoter, L-rhamnose inducible (rhaPBAD) promoter, T7 RNA polymerase promoter, trc promoter, tac promoter, lambda bacteriophage promoter (p_{L}p_{L}-9G-50), anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-Iac operator, nprM-lac operator, Vhb, Protein A, corynebacterial-E. coli like promoters, thr, horn, diphtheria toxin promoter, sig A, sig B, nusG, SoxS, katb, a-amylase (Pamy), Ptms, P43 (comprised of two overlapping RNA polymerase a factor recognition sites, σA, σB), Ptms, P43, rplK-rplA, ferredoxin promoter, and/or xylose promoter. In some embodiments, the promoter is a BBa_J23102, BBa_J23104, or BBa_J23109. In some embodiments the promoter is derived from the organism, species, or target bacterium, such as endogenous CRISPR promoter, endogenous Cas operon promoter, p16, plpp, or ptat. In some embodiments, the promoter is a bacteriophage promoter, such as the promoter for gp105 or gp245. In some embodiments, a ribosomal binding site (RBS) is provided between the promoter and the Cas system. In some embodiments, the RBS is recognized by the organism, species, or target of interest.

In some embodiments, a third step comprises providing genome-targeting spacers into the plasmid. In some embodiments, the genome-targeting spacers are identified using bioinformatics. In some embodiments, the genome-targeting spacers are provided upstream of the repeat-spacer-repeat. In some embodiments, a promoter is provided. In some embodiments, the promoter is recognized by the organism, species, or target of interest to drive the expression of the crRNA. In some embodiments, the cloning for the third step comprises using an organism or species that is not targeted by the spacer being cloned.

In some embodiments, a fourth step comprises providing a non-target spacer into the plasmid that expresses the Cas system. In some embodiments, the non-target spacer comprises a sequence that is random. In some embodiments, the non-target spacer comprises a sequence that does not comprise targeting sites in the genome of the organism, species, or target of interest. In some embodiments, the non-target spacer sequence is determined using bioinformatics to not comprise targeting sites in the genome of the organism, species, or target of interest. In some embodiments, the non-target spacer sequence is provided upstream of the repeat-spacer-repeat. In some embodiments, a promoter is provided. In some embodiments, the promoter is recognized by the organism, species, or target of interest to drive the expression of the crRNA.

In some embodiments, a fifth step comprises determining an efficacy of each spacer generated. In some embodiments, the killing efficacy is determined. In some embodiments, the efficacy of each spacer at targeting the bacterial genome is determined. In some embodiments, the plasmids comprising the spacer comprises about 0.5-fold, about 1-fold, 5-fold, 10-fold, 20-fold, 40-fold, 60-fold, 80-fold, or up to about 100 fold reduction in transfer rate as compared to a plasmid that comprises the non-targeting spacer.

### Repeat Nucleotide Sequences

In some embodiments, a repeat nucleotide sequence of the CRISPR array comprises a nucleotide sequence of any known repeat nucleotide sequence of a CRISPR-Cas system. In some embodiments, a repeat nucleotide sequence is of a synthetic sequence comprising the secondary structure of a native repeat from a CRISPR-Cas system (e.g., an internal hairpin). In some embodiments, the repeat nucleotide sequences are distinct from one another based on the known repeat nucleotide sequences of a CRISPR-Cas system. In some embodiments, the repeat nucleotide sequences are each composed of distinct secondary structures of a native repeat from a CRISPR-Cas system (e.g., an internal hairpin). In some embodiments, the repeat nucleotide sequences are a combination of distinct repeat nucleotide sequences operable with a CRISPR-Cas system.

In some embodiments, the spacer sequence is linked at its 5′ end to the 3′ end of a repeat sequence. In some embodiments, the spacer sequence is linked at its 5′ end to about 1 to about 8, about 1 to about 10, or about 1 to about 15 nucleotides of the 3′ end of a repeat sequence. In some embodiments, the about 1 to about 8, about 1 to about 10, about 1 to about 15 nucleotides of the repeat sequence are a portion of the 3′ end of a repeat sequence. In some embodiments, the spacer nucleotide sequence is linked at its 3′ end to the 5′ end of a repeat sequence. In some embodiments, the spacer is linked at its 3′ end to about 1 to about 8, about 1 to about 10, or about 1 to about 15 nucleotides of the 5′ end of a repeat sequence. In some embodiments, the about 1 to about 8, about 1 to about 10, about 1 to about 15 nucleotides of the repeat sequence are a portion of the 5′ end of a repeat sequence.

In some embodiments, the spacer nucleotide sequence is linked at its 5′ end to a first repeat sequence and linked at its 3′ end to a second repeat sequence to form a repeat-spacer-repeat sequence. In some embodiments, the spacer sequence is linked at its 5′ end to the 3′ end of a first repeat sequence and is linked at its 3′ end to the 5′ of a second repeat sequence where the spacer sequence and the second repeat sequence are repeated to form a repeat-(spacer-repeat)n sequence such that n is any integer from 1 to 100. In some embodiments, a repeat-(spacer-repeat)n sequence comprises, consists essentially of, or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

In some embodiments, the repeat sequence is identical to or substantially identical to a repeat sequence from a wild-type CRISPR loci. In some embodiments, the repeat sequence is a repeat sequence found in Table 3. In some embodiments, the repeat sequence is a sequence described herein. In some embodiments, the repeat sequence comprises a portion of a wild type repeat sequence (e.g., 1, 2, 3, 4, 5, 6, 7, 8, 9, 10, 11, 12, 13, 14, 15 or more contiguous nucleotides of a wild type repeat sequence). In some embodiments, the repeat sequence comprises, consists essentially of, or consists of at least one nucleotide (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, or more nucleotides, or any range therein). In some embodiments, the repeat sequence comprises, consists essentially of, or consists of no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 nucleotides. In some embodiments, the repeat sequence comprises about 20 to 40, 21 to 40, 22 to 40 23 to 40, 24 to 40, 25 to 40, 26 to 40, 27 to 40, 28 to 40, 29 to 40, 30 to 30, 31 to 40, 32 to 40, 33 to 40, 34 to 40, 35 to 40, 36 to 40, 37 to 40, 38 to 40, 39 to 40, 20 to 39, 20 to 38, 20 to 37, 20 to 36, 20 to 35, 20 to 34, 20 to 33, 20 to 32, 20 to 31, 20 to 30, 20 to 29, 20 to 28, 20 to 26, 20 to 25, 20 to 24, 20 to 23, 20 to 22, or 20 to 21 nucleotides. In some embodiments, the repeat sequence comprises about 20 to 35, 21 to 35, 22 to 35 23 to 35, 24 to 35, 25 to 35, 26 to 35, 27 to 35, 28 to 35, 29 to 35, 30 to 30, 31 to 35, 32 to 35, 33 to 35, 34 to 35, 25 to 40, 25 to 39, 25 to 38, 25 to 37, 25 to 36, 25 to 35, 25 to 34, 25 to 33, 25 to 32, 25 to 31, 25 to 30, 25 to 29, 25 to 28, 25 to 26 nucleotides. In some embodiments, the system is a P. aeruginosa Type I-C Cas system. In some embodiments, the P. aeruginosa Type I-C Cas system has a repeat length of about 25 to 38 nucleotides.

In some embodiments, the repeat sequence comprises at least or about 70%, 80%, 85%, 90%, 91%, 92%, 93%, 94%, 95%, 96%, 97%, 98%, 99%, or 100% sequence identity to any one of SEQ ID NOs: 13-18. In some instances, the repeat sequence comprises at least or about 95% sequence identity to any one of SEQ ID NOS: 13-18. In some instances, the repeat sequence comprises at least or about 97% sequence identity to any one of SEQ ID NOS: 13-18. In some instances, the repeat sequence comprises at least or about 99% sequence identity to any one of SEQ ID NOS: 13-18. In some instances, the repeat sequence comprises 100% sequence identity to any one of SEQ ID NOS: 13-18. In some instances, the repeat sequence comprises at least a portion having at least or about 3, 4, 5, 6, 7, 8, 9, 10, 12, 14, 16, 17, 18, 19, 20, 21, 22, 23, 24, 25, 26, 27, 28, 29, 30, 31, 32, or more than 32 nucleotides of any one of SEQ ID NOS: 13-18.

### Type I CRISPR-Cas System

In some embodiments, the Type I CRISPR-Cas system is a Type I-A system, Type I-B system, Type I-C system, Type I-D system, Type I-E system, or Type I-F system. In some embodiments, the Type I CRISPR-Cas system is a Type I-A system. In some embodiments, the Type I CRISPR-Cas system is a Type I-B system. In some embodiments, the Type I CRISPR-Cas system is a Type I-C system. In some embodiments, the Type I CRISPR-Cas system is a Type I-D system. In some embodiments, the Type I CRISPR-Cas system is a Type I-E system. In some embodiments, the Type I CRISPR-Cas system is a Type I-F system. In some embodiments, the Type I CRISPR-Cas system comprises Cascade polypeptides. Type I Cascade polypeptides process CRISPR arrays to produce a processed RNA that is then used to bind the complex to a target sequence that is complementary to the spacer in the processed RNA. In some embodiments, the Type I Cascade complex is a Type I-A Cascade polypeptides, a Type I-B Cascade polypeptides, a Type I-C Cascade polypeptides, a Type I-D Cascade polypeptides, a Type I-E Cascade polypeptides, a Type I-F Cascade polypeptides, or a Type I-U Cascade polypeptides.

In some embodiments, the Type I Cascade complex comprises: (a) a nucleotide sequence encoding a Cas7 (Csa2) polypeptide, a nucleotide sequence encoding a Cas8a1 (Csx13) polypeptide or a Cas8a2 (Csx9) polypeptide, a nucleotide sequence encoding a Cas5 polypeptide, a nucleotide sequence encoding a Csa5 polypeptide, a nucleotide sequence encoding a Cas6a polypeptide, a nucleotide sequence encoding a Cas3′ polypeptide, and a nucleotide sequence encoding a Cas3″ polypeptide having no nuclease activity (Type I-A); (b) a nucleotide sequence encoding a Cas6b polypeptide, a nucleotide sequence encoding a Cas8b (Csh1) polypeptide, a nucleotide sequence encoding a Cas7 (Csh2) polypeptide, and a nucleotide sequence encoding a Cas5 polypeptide (Type I-B); (c) a nucleotide sequence encoding a Cas5d polypeptide, a nucleotide sequence encoding a Cas8c (Csd1) polypeptide, and a nucleotide sequence encoding a Cas7 (Csd2) polypeptide (Type I-C); (d) a nucleotide sequence encoding a Cas1 Od (Csc3) polypeptide, a nucleotide sequence encoding a Csc2 polypeptide, a nucleotide sequence encoding a Csc1 polypeptide, and a nucleotide sequence encoding a Cas6d polypeptide (Type I-D); (e) a nucleotide sequence encoding a Cse1 (CasA) polypeptide, a nucleotide sequence encoding a Cse2 (CasB) polypeptide, a nucleotide sequence encoding a Cas7 (CasC) polypeptide, a nucleotide sequence encoding a Cas5 (CasD) polypeptide, and a nucleotide sequence encoding a Cas6e (CasE) polypeptide (Type I-E); and/or (f) a nucleotide sequence encoding a Cys1 polypeptide, a nucleotide sequence encoding a Cys2 polypeptide, a nucleotide sequence encoding a Cas7 (Cys3) polypeptide, and a nucleotide sequence encoding a Cas6f polypeptide (Type I-F).

In some embodiments, the Type I CRISPR-Cas system is exogenous to the Escherichia species. In some embodiments, the Type I CRISPR-Cas system is a Pseudomonas aeruginosa Type I-C Cas system (e.g. SEQ ID NO: 47-50).

### Type V CRISPR System

In some embodiments, the CRISPR-Cas system employed in the method disclosed herein is the Type V CRISPR system, using the Cpf1 nuclease (e.g., CRISPR from Prevotella and Francisella1, SEQ ID NO: 46). This monomeric protein with 1200-1500 amino acids length belongs to type V CRISPR system. Cpf1 CRISPR array consists of nine spacer sequences, which are disassociated by 36 nucleotide long repeated sequences. Cpf1 recognizes a 5′-TTTV-3′ PAM in a DNA target, which leads to the base pairing of the spacer-derived segment of the crRNA with the complementary target DNA. Since Cpf1 simultaneously possesses RNAase and DNAase activity, it does not employ tracrRNAs for crRNA biogenesis; instead, the pre-crRNA forms a pseudoknot, that is recognized and cleaved by Cpf1 itself. Cpf1 induces staggered ends (5 or 8 nucleotides 5′overhang concerning crRNA length) at the cleaved sites. AM recognition is the first step in Cpf1-mediated gene editing. When PAM is located in the surrounding of a related protospacer, it will set off subsequent hybridization of the crRNA to the target DNA strand and the formation of an R-loop structure. The PAM sequences of Cpf1 family proteins are predominantly T-rich and differ only in the number of thymidines. Also, it was revealed that the nuclease component of Cpf1 recognizes 5′-TTN-3′ PAM on the target strand. PI, REC1, and WED domains altogether participate in the PAM recognition.

### Other Antimicrobial Agents and Peptides

In some embodiments, a bacteriophage disclosed herein expresses at least one antimicrobial agent or peptide disclosed herein. In some embodiments, a bacteriophage disclosed herein is further genetically modified to express an antibacterial peptide, a functional fragment of an antibacterial peptide, and/or a lytic gene. In some embodiments, the bacteriophage comprises a nucleic acid that encodes a peptide that prevents bacteriophage degradation or a peptide that assists in breaking down or degrading biofilm matrix. In some embodiments, the bacteriophage expresses at least two antimicrobial agents or peptides disclosed herein. In some embodiments, the bacteriophage expresses at least one colicin.

In some embodiments, a bacteriophage described herein comprises a nucleic acid that encodes a peptide that prevents bacteriophage degradation or enables escape of the bacteriophage from the host defenses. In some embodiments, a bacteriophage disclosed herein comprises a nucleic acid sequence that encodes an enzybiotic where the protein product of the nucleic acid sequence targets bacteriophage resistant bacteria. In some embodiments, the peptide comprises Ipi (e.g., a sequence at least 80% identical to SEQ ID NO: 63).

In some embodiments, the bacteriophage comprises nucleic acids which encode enzymes which assist in breaking down or degrading biofilm matrix. In some embodiments, a bacteriophage disclosed herein comprises nucleic acids encoding Dispersin D aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, xylanase or lyase. In some embodiments, the enzyme is selected from the group consisting of cellulases, such as glycosyl hydroxylase family of cellulases, such as glycosyl hydroxylase 5 family of enzymes also called cellulase A; polyglucosamine (PGA) depolymerases; and colonic acid depolymerases, such as 1,4-L-fucodise hydrolase, colanic acid, depolymerazing alginase, DNase I, or combinations thereof. In some embodiments, a bacteriophage disclosed herein secretes an enzyme disclosed herein. In some embodiments, the peptide disrupts quorum sensing and biofilm formation. In some embodiments, the peptide increases the sensitivity of a bacterial cell to an antibiotic. In some embodiments, the enzyme comprises DNAse I (e.g., a sequence at least 80% identical to SEQ ID NO: 62).

In some embodiments, an antimicrobial agent or peptide is expressed and/or secreted by a bacteriophage disclosed herein. In some embodiments, the antimicrobial agent or peptide comprises PLNC8α. In some embodiments, the antimicrobial agent or peptide comprises PLNC8β. In some embodiments, the antimicrobial agent or peptide comprises LytM. In some embodiments, the antimicrobial agent or peptide comprises an anti-restriction modification enzyme.

In some embodiments, an antimicrobial agent or peptide is expressed and/or secreted by a bacteriophage disclosed herein. In some embodiments, a bacteriophage disclosed herein secretes and expresses an antibiotic such as ampicillin, penicillin, penicillin derivatives, cephalosporins, monobactams, carbapenems, ofloxacin, ciproflaxacin, levofloxacin, gatifloxacin, norfloxacin, lomefloxacin, trovafloxacin, moxifloxacin, sparfloxacin, gemifloxacin, pazufloxacin or any antibiotic disclosed herein. In some embodiments, a bacteriophage disclosed herein comprises a nucleic acid sequence encoding an antibacterial peptide, expresses an antibacterial peptide, or secretes a peptide that aids or enhances killing of a target bacterium. In some embodiments, a bacteriophage disclosed herein comprises a nucleic acid sequence encoding a peptide, a nucleic acid sequence encoding an antibacterial peptide, expresses an antibacterial peptide, or secretes a peptide that aids or enhances the activity of the first and/or the second Type I CRISPR-Cas system.

In some embodiments, the antimicrobial agent or peptide is encoded by a sequence comprising at least or about 70%, 80%, 85%, 90%, 91%, 92%, 93%, 94%, 95%, 96%, 97%, 98%, 99%, or 100% sequence identity to SEQ ID NO: 60. In some embodiments, the antimicrobial agent or peptide is encoded by a sequence comprising at least or about 70%, 80%, 85%, 90%, 91%, 92%, 93%, 94%, 95%, 96%, 97%, 98%, 99%, or 100% sequence identity to SEQ ID NO: 61.

In some embodiments, the antimicrobial agent or peptide comprises at least or about 70%, 80%, 85%, 90%, 91%, 92%, 93%, 94%, 95%, 96%, 97%, 98%, 99%, or 100% sequence identity to SEQ ID NO: 62. In some embodiments, the antimicrobial agent or peptide comprises at least or about 70%, 80%, 85%, 90%, 91%, 92%, 93%, 94%, 95%, 96%, 97%, 98%, 99%, or 100% sequence identity to SEQ ID NO: 63.

| SEQ | des- | |
|---|---|---|
| ID NO | cription | Sequence (5′-3′) |
| 60 | hDNaseI | atgttaaagattgctgcttttaacattcaaacatttggtgaaacaaaaatgagtaatgctacattagtatcat |
| | | atattgttcaaattttatctagatatgatattgctttagttcaagaagttagagatagtcatttaacagctgt |
| | | aggtaaattattagataatttaaatcaagatgctcctgatacataccattacgtagtaagtgaacctttaggt |
| | | agaaattcatataaagaaagatacttatttgtatatagacctgaccaagtaagtgctgtagattcatattatt |
| | | atgatgatggttgtgaaccttgtggtaatgatacatttaatagagaacctgctattgttagattcttttcaag |
| | | atttacagaagtaagagaatttgctattgtacctttacatgctgctcctggtgatgctgtagctgaaattgat |
| | | gctttatatgatgtatatttagatgtacaagaaaaatggggtttagaagatgtaatgttaatgggtgatttta |
| | | atgctggttgtagttatgtaagaccttcacaatggtctagtattagattatggacaagtcctacatttcaatg |
| | | gttaattcctgattcagctgatacaacagctacacctacacattgtgcttatgatagaattgtagtagctggt |
| | | atgttattaagaggtgctgtagtacctgattctgctttaccttttaattttcaagctgcttatggtttaagtg |
| | | accaattagctcaagctatttcagaccattatcctgtagaagtaatgttaaaacatcatcaccatcatcacta |
| | | a |
| 61 | Ipi | atgaaaactttcaaagaatttacttctacgactaccccggtttctactattactgaagctactcttacttctg |
| | | aagttattaaagcaaataaaggacgagaaggtaaaccgatgattagtctggttgatggtgaagaaatcaaagg |
| | | tactgtttacctaggtgatgggtggtctgctaaaaaggatggtgctacaatcgttatctctcctgctgaagaa |
| | | actgcgttgtttaaagctaaacacatttctgcggcacatctcaagattattgctaaaaatcttttgtaa |
| 62 | DNaseI | MLKIAAFNIQTFGETKMSNATLVSYIVQILSRYDIALVQEVRDSHLTAVG |
| | | KLLDNLNQDAPDTYHYVVSEPLGRNSYKERYLFVYRPDQVSAVDSYYY |
| | | DDGCEPCGNDTFNREPAIVRFFSRFTEVREFAIVPLHAAPGDAVAEIDAL |
| | | YDVYLDVQEKWGLEDVMLMGDFNAGCSYVRPSQWSSIRLWTSPTFQW |
| | | LIPDSADTTATPTHCAYDRIVVAGMLLRGAVVPDSALPFNFQAAYGLSD |
| | | QLAQAISDHYPVEVMLKHHHHHH* |
| 63 | Ipi | MKTFKEFTSTTTPVSTITEATLTSEVIKANKGREGKPMISLVDGEEIKGTV |
| | | YLGDGWSAKKDGATIVISPAEETALFKAKHISAAHLKIIAKNLL* |

### Bacteriophage

In some embodiments, the bacteriophage is an obligate lytic bacteriophage. In some P21D embodiments, the bacteriophage is a temperate bacteriophage with retained lysogeny genes. In some embodiments, the bacteriophage is a temperate bacteriophage with some lysogeny genes removed, replaced, or inactivated. In some embodiments, the bacteriophage is a temperate bacteriophage with a lysogeny gene removed, replaced, or inactivated, thereby rendering the bacteriophage lytic.

In some embodiments, the bacteriophage targets Escherichia spp. In some embodiments, the bacteriophage targets Escherichia coli. In some embodiments, the bacteriophage specifically targets Escherichia spp. over other bacterial species. In some embodiments, the bacteriophage targets Escherichia spp. in the absence of a CRISPR-Cas system.

In some embodiments, the bacteriophage is a Tequatrovirus, a Mosigvirus, a Phapecoctavirus, a Unique Myoviridae, a Vectrevirus, or a Tequintavirus. In some embodiments, the bacteriophage is a Tequatrovirus. In some embodiments, the bacteriophage is a Mosigyvirus. In some embodiments, the bacteriophage is a Phapecoctavirus. In some embodiments, the bacteriophage is a Unique Myoviridae. In some embodiments, the bacteriophage is a Vectrevirus. In some embodiments, the bacteriophage is a Tequintavirus. In some embodiments, the bacteriophage comprises a CRISPR-Cas3 system. In some embodiments, the bacteriophage comprises a colicin.

In some embodiments, the bacteriophage is p004Ke009, p00c0e030, p00c0e103, p00exe014, p004ke007, p004Ke005, p004K, p00c0, p00ex, p00jc, p00ke, or p5516, which target Escherichia ssp. In some embodiments, the bacteriophages include, but are not limited to, p004ke007, p004Ke005, p004K, p00c0, p00ex, p00jc, p00ke, or p5516. In some embodiments, “a bacteriophage” is inclusive of one or more bacteriophage, where a first bacteriophage and a second bacteriophage are different or the same. For instance, a bacteriophage comprises p004Ke009, p00c0e030, p00c0e030, and p00exe014.

In some embodiments, the bacteriophage is p004K (ATCC Accession No. PTA-127149), or a mutant thereof which retains the ability to target Escherichia spp. In some embodiments, the bacteriophage comprises at least 70%, 75%, 80%, 85%, 90% 95%, or 100% sequence identity with p004K. In some embodiments, the bacteriophage is a p004K bacteriophage comprising a CRISPR system. In some embodiments, the bacteriophage comprises at least 70%, 75%, 80%, 85%, 90% 95%, or 100% sequence identity with p004ke005. In some embodiments, the bacteriophage comprises at least 70%, 75%, 80%, 85%, 90% 95%, or 100% sequence identity with p004ke007. In some embodiments, the bacteriophage comprises at least 70%, 75%, 80%, 85%, 90% 95%, or 100% sequence identity with p004ke009 (ATCC Accession No. PTA-127150). In some embodiments, the bacteriophage is a p004K bacteriophage comprising a colicin as described herein. In some embodiments, the bacteriophage comprises at least 70%, 75%, 80%, 85%, 90% 95%, or 100% sequence identity with p004ke127 (ATCC Accession No. PTA-127580). In some embodiments, the bacteriophage comprises at least at least 70%, 75%, 80%, 85%, 90% 95%, or 100% sequence identity with p004ke124.

In some embodiments, the bacteriophage is p00c0 (ATCC Accession No. PTA-127143), or a mutant thereof which retains the ability to target Escherichia spp. In some embodiments, the bacteriophage comprises at least 70%, 75%, 80%, 85%, 90% 95%, or 100% sequence identity with p00c0. In some embodiments, the bacteriophage is a p00c0 bacteriophage comprising a CRISPR system. In some embodiments, the bacteriophage comprises at least 70%, 75%, 80%, 85%, 90% 95%, or 100% sequence identity with p00c0e030 (ATCC Accession No. PTA-127144). In some embodiments, the bacteriophage comprises at least 70%, 75%, 80%, 85%, 90% 95%, or 100% sequence identity with p00c0e103. In some embodiments, the bacteriophage is a p00c0e bacteriophage comprising a colicin as described herein.

In some embodiments, the bacteriophage is p00ex (ATCC Accession No. PTA-127145), or a mutant thereof which retains the ability to target Escherichia spp. In some embodiments, the bacteriophage comprises at least 70%, 75%, 80%, 85%, 90% 95%, or 100% sequence identity with p00ex. In some embodiments, the bacteriophage is a p00ex bacteriophage comprising a CRISPR system. In some embodiments, the bacteriophage comprises at least 70%, 75%, 80%, 85%, 90% 95%, or 100% sequence identity with p00exe014 (ATCC Accession No. PTA-127146). In some embodiments, the bacteriophage is a p00ex bacteriophage comprising a colicin as described herein. In some embodiments, the bacteriophage comprises at least 70%, 75%, 80%, 85%, 90% 95%, or 100% sequence identity with p00exe299 (ATCC Accession No. PTA-127578). In some embodiments, the bacteriophage comprises at least 70%, 75%, 80%, 85%, 90% 95%, or 100% sequence identity with p00exe296 (ATCC Accession No. PTA-127582). In some embodiments, the bacteriophage comprises at least 70%, 75%, 80%, 85%, 90% 95%, or 100% sequence identity with p00exe291. In some embodiments, the bacteriophage comprises at least 70%, 75%, 80%, 85%, 90% 95%, or 100% sequence identity with p00exe300.

In some embodiments, the bacteriophage is p00jc (ATCC Accession No. PTA-127147), or a mutant thereof which retains the ability to target Escherichia spp. In some embodiments, the bacteriophage comprises at least 70%, 75%, 80%, 85%, 90% 95%, or 100% sequence identity with p00jc. In some embodiments, the bacteriophage is a p00jc bacteriophage comprising a CRISPR system. In some embodiments, the bacteriophage is a p00jc bacteriophage comprising a colicin as described herein. In some embodiments, the bacteriophage comprises at least 70%, 75%, 80%, 85%, 90% 95%, or 100% sequence identity with p00jce098 (ATCC Accession No. PTA-127581). In some embodiments, the bacteriophage comprises at least 70%, 75%, 80%, 85%, 90%, 95%, or 100% sequence identity with p00jce172.

In some embodiments, the bacteriophage is p00ke (ATCC Accession No. PTA-127148), or a mutant thereof which retains the ability to target Escherichia spp. In some embodiments, the bacteriophage comprises at least 70%, 75%, 80%, 85%, 90% 95%, or 100% sequence identity with p00ke. In some embodiments, the bacteriophage is a p00ke bacteriophage comprising a CRISPR system. In some embodiments, the bacteriophage is a p00ke bacteriophage comprising a colicin as described herein. In some embodiments, the bacteriophage comprises at least 70%, 75%, 80%, 85%, 90% 95%, or 100% sequence identity with p00kee072.

In some embodiments, the bacteriophage is p5516 (ATCC Accession No PTA-127151), or a mutant thereof which retains the ability to target Escherichia spp. In some embodiments, the bacteriophage comprises at least 70%, 75%, 80%, 85%, 90% 95%, or 100% sequence identity with p5516. In some embodiments, the bacteriophage is a p5516 bacteriophage comprising a CRISPR system. In some embodiments, the bacteriophage is a p5516 bacteriophage comprising a colicin as described herein.

In some embodiments, the bacteriophage is p6984 (ATCC Accession No PTA-127578), or a mutant thereof which retains the ability to target Escherichia spp. In some embodiments, the bacteriophage comprises at least 70%, 75%, 80%, 85%, 90% 95%, or 100% sequence identity with p6984. In some embodiments, the bacteriophage is a p6984 bacteriophage comprising a CRISPR system. In some embodiments, the bacteriophage is a p6984 bacteriophage comprising a colicin as described herein.

In some embodiments, the bacteriophage is p6921 (ATCC Accession No PTA-127576), or a mutant thereof which retains the ability to target Escherichia spp. In some embodiments, the bacteriophage comprises at least 70%, 75%, 80%, 85%, 90% 95%, or 100% sequence identity with p6921. In some embodiments, the bacteriophage is a p6921 bacteriophage comprising a CRISPR system. In some embodiments, the bacteriophage is a p6921 bacteriophage comprising a colicin as described herein.

In some embodiments, the bacteriophage is p6977 (ATCC Accession No PTA-127577), or a mutant thereof which retains the ability to target Escherichia spp. In some embodiments, the bacteriophage comprises at least 70%, 75%, 80%, 85%, 90% 95%, or 100% sequence identity with p5516. In some embodiments, the bacteriophage is a p6977 bacteriophage comprising a CRISPR system. In some embodiments, the bacteriophage is a p6977 bacteriophage comprising a colicin as described herein.

In some embodiments, described herein is a recombinant Tequatrovirus comprising a nucleic acid sequence encoding Colicin U. In some embodiments, the Tequatrovirus has at least 70%, 75%, 80%, 85%, 90% 95%, or 100% sequence identity with p00ex. In some embodiments, the recombinant Tequatrovirus is a recombinant p00ex. In some embodiments the recombinant bacteriophage virus has at least 70%, 75%, 80%, 85%, 90% 95%, or 100% sequence identity with p00ex. In some embodiments, the Colicin U has at least 70%, 75%, 80%, 85%, 90% 95%, or 100% identity to SEQ ID NO 168. In some embodiments, Colicin U is a homolog of the Colicin U having SEQ ID NO: 163. In some embodiments, the nucleic acid encoding Colicin U is at least 70%, 75%, 80%, 85%, 90% 95%, or 100% identical to SEQ ID NO: 163, and the recombinant Tequatrovirus bacteriophage is at least 70%, 75%, 80%, 85%, 90% 95%, or 100% identical to p00ex (ATCC Accession No. PTA-127145). In some embodiments, the Tequatrovirus has at least 70%, 75%, 80%, 85%, 90% 95%, or 100% sequence identity with p004k. In some embodiments, the recombinant Tequatrovirus is a recombinant p004k. In some embodiments the recombinant bacteriophage virus has at least 70%, 75%, 80%, 85%, 90% 95%, or 100% sequence identity with p004k. In some embodiments, the Colicin U has at least 70%, 75%, 80%, 85%, 90% 95%, or 100% identity to SEQ ID NO 168. In some embodiments, Colicin U is a homolog of the Colicin U having SEQ ID NO: 163. In some embodiments, the nucleic acid encoding Colicin U is at least 70%, 75%, 80%, 85%, 90% 95%, or 100% identical to SEQ ID NO: 163, and the recombinant Tequatrovirus bacteriophage is at least 70%, 75%, 80%, 85%, 90% 95%, or 100% identical to p004k (ATCC Accession No. PTA-127149).

In some embodiments, described herein is a recombinant Tequatrovirus comprising a nucleic acid sequence encoding Colicin K. In some embodiments, the Tequatrovirus has at least 70%, 75%, 80%, 85%, 90% 95%, or 100% sequence identity with p00ex. In some embodiments, the recombinant Tequatrovirus is a recombinant p00ex. In some embodiments the recombinant bacteriophage virus has at least 70%, 75%, 80%, 85%, 90% 95%, or 100% sequence identity with p00ex. In some embodiments, the Colicin K has at least 70%, 75%, 80%, 85%, 90% 95%, or 100% identity to SEQ ID NO 167. In some embodiments, Colicin K is a homolog of the Colicin K having SEQ ID NO: 161. In some embodiments, the nucleic acid encoding Colicin K is at least 70%, 75%, 80%, 85%, 90% 95%, or 100% identical to SEQ ID NO: 161, and the recombinant Tequatrovirus bacteriophage is at least 70%, 75%, 80%, 85%, 90% 95%, or 100% identical to p00ex (ATCC Accession No. PTA-127145). In some embodiments, the Tequatrovirus has at least 70%, 75%, 80%, 85%, 90% 95%, or 100% sequence identity with p004k. In some embodiments, the recombinant Tequatrovirus is a recombinant p004k. In some embodiments the recombinant bacteriophage virus has at least 70%, 75%, 80%, 85%, 90% 95%, or 100% sequence identity with p004k. In some embodiments, the Colicin K has at least 70%, 75%, 80%, 85%, 90% 95%, or 100% identity to SEQ ID NO 167. In some embodiments, Colicin K is a homolog of the Colicin K having SEQ ID NO: 161. In some embodiments, the nucleic acid encoding Colicin K is at least 70%, 75%, 80%, 85%, 90% 95%, or 100% identical to SEQ ID NO: 161, and the recombinant Tequatrovirus bacteriophage is at least 70%, 75%, 80%, 85%, 90% 95%, or 100% identical to p004k (ATCC Accession No. PTA-127149).

In some embodiments, described herein is a recombinant Tequatrovirus comprising a nucleic acid sequence encoding Colicin 10. In some embodiments, the Tequatrovirus has at least 70%, 75%, 80%, 85%, 90% 95%, or 100% sequence identity with p00ex. In some embodiments, the recombinant Tequatrovirus is a recombinant p00ex. In some embodiments the recombinant bacteriophage virus has at least 70%, 75%, 80%, 85%, 90% 95%, or 100% sequence identity with p00ex. In some embodiments, the Colicin 10 has at least 70%, 75%, 80%, 85%, 90% 95%, or 100% identity to SEQ ID NO 166. In some embodiments, Colicin 10 is a homolog of the Colicin 10 having SEQ ID NO: 161. In some embodiments, the nucleic acid encoding Colicin 10 is at least 70%, 75%, 80%, 85%, 90% 95%, or 100% identical to SEQ ID NO: 161, and the recombinant Tequatrovirus bacteriophage is at least 70%, 75%, 80%, 85%, 90% 95%, or 100% identical to p00ex (ATCC Accession No. PTA-127145). In some embodiments, the Tequatrovirus has at least 70%, 75%, 80%, 85%, 90% 95%, or 100% sequence identity with p004k. In some embodiments, the recombinant Tequatrovirus is a recombinant p004k. In some embodiments the recombinant bacteriophage virus has at least 70%, 75%, 80%, 85%, 90% 95%, or 100% sequence identity with p004k. In some embodiments, the Colicin 10 has at least 70%, 75%, 80%, 85%, 90% 95%, or 100% identity to SEQ ID NO 166. In some embodiments, Colicin 10 is a homolog of the Colicin 10 having SEQ ID NO: 162. In some embodiments, the nucleic acid encoding Colicin 10 is at least 70%, 75%, 80%, 85%, 90% 95%, or 100% identical to SEQ ID NO: 162, and the recombinant Tequatrovirus bacteriophage is at least 70%, 75%, 80%, 85%, 90% 95%, or 100% identical to p004k (ATCC Accession No. PTA-127149).

In some embodiments, described herein is a recombinant Tequatrovirus comprising a nucleic acid sequence encoding Colicin Ib. In some embodiments, the Tequatrovirus has at least 70%, 75%, 80%, 85%, 90% 95%, or 100% sequence identity with p00ex. In some embodiments, the recombinant Tequatrovirus is a recombinant p00ex. In some embodiments the recombinant bacteriophage virus has at least 70%, 75%, 80%, 85%, 90% 95%, or 100% sequence identity with p00ex. In some embodiments, the Colicin Ib has at least 70%, 75%, 80%, 85%, 90% 95%, or 100% identity to SEQ ID NO 165. In some embodiments, Colicin Ib is a homolog of the Colicin Ib having SEQ ID NO: 161. In some embodiments, the nucleic acid encoding Colicin Ib is at least 70%, 75%, 80%, 85%, 90% 95%, or 100% identical to SEQ ID NO: 161, and the recombinant Tequatrovirus bacteriophage is at least 70%, 75%, 80%, 85%, 90% 95%, or 100% identical to p00ex (ATCC Accession No. PTA-127145). In some embodiments, the Tequatrovirus has at least 70%, 75%, 80%, 85%, 90% 95%, or 100% sequence identity with p004k. In some embodiments, the recombinant Tequatrovirus is a recombinant p004k. In some embodiments the recombinant bacteriophage virus has at least 70%, 75%, 80%, 85%, 90% 95%, or 100% sequence identity with p004k. In some embodiments, the Colicin Ib has at least 70%, 75%, 80%, 85%, 90% 95%, or 100% identity to SEQ ID NO 165. In some embodiments, Colicin Ib is a homolog of the Colicin Ib having SEQ ID NO: 161. In some embodiments, the nucleic acid encoding Colicin Ib is at least 70%, 75%, 80%, 85%, 90% 95%, or 100% identical to SEQ ID NO: 161, and the recombinant Tequatrovirus bacteriophage is at least 70%, 75%, 80%, 85%, 90% 95%, or 100% identical to p004k (ATCC Accession No. PTA-127149).

In some embodiments, described herein is a recombinant Mosigvirus comprising a nucleic acid sequence encoding Colicin U. In some embodiments, the Mosigvirus has at least 70%, 75%, 80%, 85%, 90% 95%, or 100% sequence identity with p00c0. In some embodiments, the recombinant Mosigvirus is a recombinant p00c0. In some embodiments the recombinant bacteriophage virus has at least 70%, 75%, 80%, 85%, 90% 95%, or 100% sequence identity with p00c0. In some embodiments, the Colicin U has at least 70%, 75%, 80%, 85%, 90% 95%, or 100% identity to SEQ ID NO 168. In some embodiments, Colicin U is a homolog of the Colicin U having SEQ ID NO: 163. In some embodiments, the nucleic acid encoding Colicin U is at least 70%, 75%, 80%, 85%, 90% 95%, or 100% identical to SEQ ID NO: 163, and the recombinant Mosigvirus bacteriophage is at least 70%, 75%, 80%, 85%, 90% 95%, or 100% identical to p00c0 (ATCC Accession No. PTA-127143).

In some embodiments, described herein is a recombinant Mosigvirus comprising a nucleic acid sequence encoding Colicin K. In some embodiments, the Mosigvirus has at least 70%, 75%, 80%, 85%, 90% 95%, or 100% sequence identity with p00c0. In some embodiments, the recombinant Mosigvirus is a recombinant p00c0. In some embodiments the recombinant bacteriophage virus has at least 70%, 75%, 80%, 85%, 90% 95%, or 100% sequence identity with p00c0. In some embodiments, the Colicin K has at least 70%, 75%, 80%, 85%, 90% 95%, or 100% identity to SEQ ID NO 167. In some embodiments, Colicin K is a homolog of the Colicin K having SEQ ID NO: 161. In some embodiments, the nucleic acid encoding Colicin K is at least 70%, 75%, 80%, 85%, 90% 95%, or 100% identical to SEQ ID NO: 161, and the recombinant Mosigvirus bacteriophage is at least 70%, 75%, 80%, 85%, 90% 95%, or 100% identical to p00c0 (ATCC Accession No. PTA-127143).

In some embodiments, described herein is a recombinant Mosigvirus comprising a nucleic acid sequence encoding Colicin 10. In some embodiments, the Mosigvirus has at least 70%, 75%, 80%, 85%, 90% 95%, or 100% sequence identity with p00c0. In some embodiments, the recombinant Mosigvirus is a recombinant p00c0. In some embodiments the recombinant bacteriophage virus has at least 70%, 75%, 80%, 85%, 90% 95%, or 100% sequence identity with p00c0. In some embodiments, the Colicin 10 has at least 70%, 75%, 80%, 85%, 90% 95%, or 100% identity to SEQ ID NO 166. In some embodiments, Colicin 10 is a homolog of the Colicin 10 having SEQ ID NO: 161. In some embodiments, the nucleic acid encoding Colicin 10 is at least 70%, 75%, 80%, 85%, 90% 95%, or 100% identical to SEQ ID NO: 161, and the recombinant Mosigvirus bacteriophage is at least 70%, 75%, 80%, 85%, 90% 95%, or 100% identical to p00c0 (ATCC Accession No. PTA-127143).

In some embodiments, described herein is a recombinant Mosigvirus comprising a nucleic acid sequence encoding Colicin Ib. In some embodiments, the Mosigvirus has at least 70%, 75%, 80%, 85%, 90% 95%, or 100% sequence identity with p00c0. In some embodiments, the recombinant Mosigvirus is a recombinant p00c0. In some embodiments the recombinant bacteriophage virus has at least 70%, 75%, 80%, 85%, 90% 95%, or 100% sequence identity with p00c0. In some embodiments, the Colicin Ib has at least 70%, 75%, 80%, 85%, 90% 95%, or 100% identity to SEQ ID NO 165. In some embodiments, Colicin Ib is a homolog of the Colicin Ib having SEQ ID NO: 161. In some embodiments, the nucleic acid encoding Colicin Ib is at least 70%, 75%, 80%, 85%, 90% 95%, or 100% identical to SEQ ID NO: 161, and the recombinant Mosigvirus bacteriophage is at least 70%, 75%, 80%, 85%, 90% 95%, or 100% identical to p00c0 (ATCC Accession No. PTA-127143).

In some embodiments, described herein is a recombinant Phapecoctavirus comprising a nucleic acid sequence encoding Colicin U. In some embodiments, the Phapecoctavirus has at least 70%, 75%, 80%, 85%, 90% 95%, or 100% sequence identity with p00jc. In some embodiments, the recombinant Phapecoctavirus is a recombinant p00jc. In some embodiments the recombinant bacteriophage virus has at least 70%, 75%, 80%, 85%, 90% 95%, or 100% sequence identity with p00jc. In some embodiments, the Colicin U has at least 70%, 75%, 80%, 85%, 90% 95%, or 100% identity to SEQ ID NO 168. In some embodiments, Colicin U is a homolog of the Colicin U having SEQ ID NO: 163. In some embodiments, the nucleic acid encoding Colicin U is at least 70%, 75%, 80%, 85%, 90% 95%, or 100% identical to SEQ ID NO: 163, and the recombinant Phapecoctavirus bacteriophage is at least 70%, 75%, 80%, 85%, 90% 95%, or 100% identical to p00jc (ATCC Accession No. PTA-127147).

In some embodiments, described herein is a recombinant Phapecoctavirus comprising a nucleic acid sequence encoding Colicin K. In some embodiments, the Phapecoctavirus has at least 70%, 75%, 80%, 85%, 90% 95%, or 100% sequence identity with p00jc. In some embodiments, the recombinant Phapecoctavirus is a recombinant p00jc. In some embodiments the recombinant bacteriophage virus has at least 70%, 75%, 80%, 85%, 90% 95%, or 100% sequence identity with p00jc. In some embodiments, the Colicin K has at least 70%, 75%, 80%, 85%, 90% 95%, or 100% identity to SEQ ID NO 167. In some embodiments, Colicin K is a homolog of the Colicin K having SEQ ID NO: 161. In some embodiments, the nucleic acid encoding Colicin K is at least 70%, 75%, 80%, 85%, 90% 95%, or 100% identical to SEQ ID NO: 161, and the recombinant Phapecoctavirus bacteriophage is at least 70%, 75%, 80%, 85%, 90% 95%, or 100% identical to p00jc (ATCC Accession No. PTA-127147).

In some embodiments, described herein is a recombinant Phapecoctavirus comprising a nucleic acid sequence encoding Colicin 10. In some embodiments, the Phapecoctavirus has at least 70%, 75%, 80%, 85%, 90% 95%, or 100% sequence identity with p00jc. In some embodiments, the recombinant Phapecoctavirus is a recombinant p00jc. In some embodiments the recombinant bacteriophage virus has at least 70%, 75%, 80%, 85%, 90% 95%, or 100% sequence identity with p00jc. In some embodiments, the Colicin 10 has at least 70%, 75%, 80%, 85%, 90% 95%, or 100% identity to SEQ ID NO 166. In some embodiments, Colicin 10 is a homolog of the Colicin 10 having SEQ ID NO: 161. In some embodiments, the nucleic acid encoding Colicin 10 is at least 70%, 75%, 80%, 85%, 90% 95%, or 100% identical to SEQ ID NO: 161, and the recombinant Phapecoctavirus bacteriophage is at least 70%, 75%, 80%, 85%, 90% 95%, or 100% identical to p00jc (ATCC Accession No. PTA-127147).

In some embodiments, described herein is a recombinant Phapecoctavirus comprising a nucleic acid sequence encoding Colicin Ib. In some embodiments, the Phapecoctavirus has at least 70%, 75%, 80%, 85%, 90% 95%, or 100% sequence identity with p00jc. In some embodiments, the recombinant Phapecoctavirus is a recombinant p00jc. In some embodiments the recombinant bacteriophage virus has at least 70%, 75%, 80%, 85%, 90% 95%, or 100% sequence identity with p00jc. In some embodiments, the Colicin Ib has at least 70%, 75%, 80%, 85%, 90% 95%, or 100% identity to SEQ ID NO 165. In some embodiments, Colicin Ib is a homolog of the Colicin Ib having SEQ ID NO: 161. In some embodiments, the nucleic acid encoding Colicin Ib is at least 70%, 75%, 80%, 85%, 90% 95%, or 100% identical to SEQ ID NO: 161, and the recombinant Phapecoctavirus bacteriophage is at least 70%, 75%, 80%, 85%, 90% 95%, or 100% identical to p00jc (ATCC Accession No. PTA-127147).

In some embodiments, described herein is a recombinant Unique Myoviridae comprising a nucleic acid sequence encoding Colicin U. In some embodiments, the Unique Myoviridae has at least 70%, 75%, 80%, 85%, 90% 95%, or 100% sequence identity with p00ke. In some embodiments, the recombinant Unique Myoviridae is a recombinant p00ke. In some embodiments the recombinant bacteriophage virus has at least 70%, 75%, 80%, 85%, 90% 95%, or 100% sequence identity with p00ke. In some embodiments, the Colicin U has at least 70%, 75%, 80%, 85%, 90% 95%, or 100% identity to SEQ ID NO 168. In some embodiments, Colicin U is a homolog of the Colicin U having SEQ ID NO: 163. In some embodiments, the nucleic acid encoding Colicin U is at least 70%, 75%, 80%, 85%, 90% 95%, or 100% identical to SEQ ID NO: 163, and the recombinant Unique Myoviridae bacteriophage is at least 70%, 75%, 80%, 85%, 90% 95%, or 100% identical to p00ke (ATCC Accession No. PTA-127148).

In some embodiments, described herein is a recombinant Unique Myoviridae comprising a nucleic acid sequence encoding Colicin K. In some embodiments, the Unique Myoviridae has at least 70%, 75%, 80%, 85%, 90% 95%, or 100% sequence identity with p00ke. In some embodiments, the recombinant Unique Myoviridae is a recombinant p00ke. In some embodiments the recombinant bacteriophage virus has at least 70%, 75%, 80%, 85%, 90% 95%, or 100% sequence identity with p00ke. In some embodiments, the Colicin K has at least 70%, 75%, 80%, 85%, 90% 95%, or 100% identity to SEQ ID NO 167. In some embodiments, Colicin K is a homolog of the Colicin K having SEQ ID NO: 161. In some embodiments, the nucleic acid encoding Colicin K is at least 70%, 75%, 80%, 85%, 90% 95%, or 100% identical to SEQ ID NO: 161, and the recombinant Unique Myoviridae bacteriophage is at least 70%, 75%, 80%, 85%, 90% 95%, or 100% identical to p00ke (ATCC Accession No. PTA-127148).

In some embodiments, described herein is a recombinant Unique Myoviridae comprising a nucleic acid sequence encoding Colicin 10. In some embodiments, the Unique Myoviridae has at least 70%, 75%, 80%, 85%, 90% 95%, or 100% sequence identity with p00ke. In some embodiments, the recombinant Unique Myoviridae is a recombinant p00ke. In some embodiments the recombinant bacteriophage virus has at least 70%, 75%, 80%, 85%, 90% 95%, or 100% sequence identity with p00ke. In some embodiments, the Colicin 10 has at least 70%, 75%, 80%, 85%, 90% 95%, or 100% identity to SEQ ID NO 166. In some embodiments, Colicin 10 is a homolog of the Colicin 10 having SEQ ID NO: 161. In some embodiments, the nucleic acid encoding Colicin 10 is at least 70%, 75%, 80%, 85%, 90% 95%, or 100% identical to SEQ ID NO: 161, and the recombinant Unique Myoviridae bacteriophage is at least 70%, 75%, 80%, 85%, 90% 95%, or 100% identical to p00ke (ATCC Accession No. PTA-127148).

In some embodiments, described herein is a recombinant Unique Myoviridae comprising a nucleic acid sequence encoding Colicin Ib. In some embodiments, the Unique Myoviridae has at least 70%, 75%, 80%, 85%, 90% 95%, or 100% sequence identity with p00ke. In some embodiments, the recombinant Unique Myoviridae is a recombinant p00ke. In some embodiments the recombinant bacteriophage virus has at least 70%, 75%, 80%, 85%, 90% 95%, or 100% sequence identity with p00ke. In some embodiments, the Colicin Ib has at least 70%, 75%, 80%, 85%, 90% 95%, or 100% identity to SEQ ID NO 165. In some embodiments, Colicin Ib is a homolog of the Colicin Ib having SEQ ID NO: 161. In some embodiments, the nucleic acid encoding Colicin Ib is at least 70%, 75%, 80%, 85%, 90% 95%, or 100% identical to SEQ ID NO: 161, and the recombinant Unique Myoviridae bacteriophage is at least 70%, 75%, 80%, 85%, 90% 95%, or 100% identical to p00ke (ATCC Accession No. PTA-127148).

In some embodiments, the bacteriophage comprises a bacteriophage listed in Table 1A, or a mutant thereof, which retains the ability to target Escherichia spp.

### Bacteriophage Cocktails

Also disclosed herein is a cocktail comprising two or more bacteriophage. In some embodiments, the two or more bacteriophage are selected from the lineage consisting of a Tequatrovirus, a Mosigyvirus, a Phapecoctavirus, a Unique Myoviridae, a Vectrevirus, or a Tequintavirus. In some embodiments, the cocktail comprises at least six bacteriophage, wherein the bacteriophage comprise a Tequatrovirus, a Mosigyvirus, a Phapecoctavirus, a Unique Myoviridae, and a vectrevirus. In some embodiments, at least one bacteriophage of the cocktail comprises a colicin. In some embodiments, at least two bacteriophages of the cocktail comprise a colicin. In some embodiments, at least three bacteriophage of the cocktail comprise a colicin. In some embodiments, at least four bacteriophage of the cocktail comprise a colicin. In some embodiments, at least one bacteriophage of the cocktail does not comprise a colicin. In some embodiments, at least two bacteriophages of the cocktail do not comprise a colicin. In some embodiments, at least three bacteriophage of the cocktail do not comprise a colicin. In some embodiments, at least one bacteriophage of the cocktail comprises a CRISPR-Cas system. In some embodiments, at least two bacteriophages of the cocktail comprise a CRISPR-Cas system. In some embodiments, at least three bacteriophage of the cocktail comprise a CRISPR-Cas system. In some embodiments, at least four bacteriophage of the cocktail comprise a CRISPR-Cas system. In some embodiments, at least one bacteriophage of the cocktail does not comprise a CRISPR-Cas system. In some embodiments, at least two bacteriophages of the cocktail do not comprise a CRISPR-Cas system. In some embodiments, at least three bacteriophages of the cocktail do not comprise a CRISPR-Cas system. In some embodiments, at least one bacteriophage of the cocktail does not comprise a CRISPR-Cas system and does not comprise a colicin. In some embodiments, the cocktail comprises a cocktail described in Table 1B or Table 36.

In some embodiments, the bacteriophage cocktail has a host range greater than that of an individual bacteriophage. The increased host range may allow for targeting a large number of strains of E. coli. In some embodiments, the bacteriophage cocktail targets at least 70%, 75%, 80%, 85%, 90%, 95%, 97.5%, 99% or more than 99% of strains of E. coli. In some embodiments, the bacteriophage cocktail targets at least 50, 100, 150, 200, 250, 300, 3500, 400, 450, 500 or more than 500 strains of E. coli. In some embodiments, the bacteriophage cocktail targets E. coli specifically and does not target other species of bacteria. In some embodiments, the bacteriophage cocktail targets E. coli and does not broadly target other Enterobacteriaceae. In some embodiments, the bacteriophage targets E. coli and does not broadly target Shigella spp, Salmonella spp, Citrobacter spp, or a combination thereof.

In some embodiments, the cocktail comprises at least two bacteriophage, wherein the bacteriophage comprise p00ex, p004k, p00ke, p00c0, p00jc, p5516, p6921, p6977, and p6984. In some embodiments, the cocktail comprises a first bacteriophage comprising at least 70%, 75%, 80%, 85%, 90%, 95%, or 100% sequence identity with p00ex. In some embodiments, the cocktail comprises a second bacteriophage comprising at least 70%, 75%, 80%, 85%, 90%, 95%, or 100% sequence identity with p0004k. In some embodiments, the cocktail comprises a third bacteriophage comprising at least 70%, 75%, 80%, 85%, 90%, 95%, or 100% sequence identity with p00ke. In some embodiments, the cocktail comprises a fourth bacteriophage comprising at least 70%, 75%, 80%, 85%, 90%, 95%, or 100% sequence identity with p00c0. In some embodiments, the cocktail comprises a fifth bacteriophage comprising at least 70%, 75%, 80%, 85%, 90%, 95%, or 100% sequence identity with p00jc. In some embodiments, the cocktail comprises a sixth bacteriophage comprising at least 70%, 75%, 80%, 85%, 90%, 95%, or 100% sequence identity with p6921. In some embodiments, the cocktail comprises a seventh bacteriophage comprising at least 70%, 75%, 80%, 85%, 90%, 95%, or 100% sequence identity with p6984. In some embodiments, the cocktail comprises an eighth bacteriophage comprising at least 70%, 75%, 80%, 85%, 90%, 95%, or 100% sequence identity with p6921. In some embodiments, the cocktail comprises a ninth bacteriophage comprising at least 70%, 75%, 80%, 85%, 90%, 95%, or 100% sequence identity with p6977. In some embodiments, at least one, two, three, four, or five bacteriophage comprise a CRISPR-Cas system. In some embodiments, at least one, two, or three bacteriophage do not comprise a CRISPR Cas system. In some embodiments, at least one, two, three, four, or five bacteriophage comprise a nucleic acid encoding a colicin. In some embodiments, the colicin comprises Colicin U, Colicin K, Colicin 10, Colicin Ib, or a homolog thereof.

In some embodiments, the cocktail comprises at least two bacteriophage, wherein the bacteriophage comprise p00ex, p0004k, p00ke, p00c0, p00jc, p5516, or p6921. In some embodiments, the cocktail comprises a first bacteriophage comprising at least 70%, 75%, 80%, 85%, 90%, 95%, or 100% sequence identity with p00ex. In some embodiments, the cocktail comprises a second bacteriophage comprising at least 70%, 75%, 80%, 85%, 90%, 95%, or 100% sequence identity with p0004k. In some embodiments, the cocktail comprises a third bacteriophage comprising at least 70%, 75%, 80%, 85%, 90%, 95%, or 100% sequence identity with p00ke. In some embodiments, the cocktail comprises a fourth bacteriophage comprising at least 70%, 75%, 80%, 85%, 90%, 95%, or 100% sequence identity with p00c0. In some embodiments, the cocktail comprises a fifth bacteriophage comprising at least 70%, 75%, 80%, 85%, 90%, 95%, or 100% sequence identity with p00jc. In some embodiments, the cocktail comprises a sixth bacteriophage comprising at least 70%, 75%, 80%, 85%, 90%, 95%, or 100% sequence identity with p6921. In some embodiments, the cocktail comprises a seventh bacteriophage comprising at least 70%, 75%, 80%, 85%, 90%, 95%, or 100% sequence identity with p6984. In some embodiments, at least one, two, three, four, or five bacteriophage comprise a CRISPR-Cas system. In some embodiments, at least one, two, or three bacteriophage do not comprise a CRISPR Cas system. In some embodiments, at least one, two, three, four, or five bacteriophage comprise a nucleic acid encoding a colicin. In some embodiments, the colicin comprises Colicin U, Colicin K, Colicin 10, Colicin Ib, or a homolog thereof.

In some embodiments, the cocktail comprises a first recombinant bacteriophage comprising at least 70%, 75%, 80%, 85%, 90%, 95%, or 100% sequence identity with p6921; and (b) a second bacteriophage comprising at least 70%, 75%, 80%, 85%, 90%, 95%, or 100% sequence identity with p5516, p00exe014, p004Ke009, p00jc, p00ke, or p00c0e030. In some embodiments, the cocktail comprises a third bacteriophage comprising at least 70%, 75%, 80%, 85%, 90%, 95%, or 100% sequence identity with p5516, p00exe014, p004Ke009, p00jc, p00ke, or p00c0e030. In some embodiments, the cocktail comprises a fourth bacteriophage comprising at least 70%, 75%, 80%, 85%, 90%, 95%, or 100% sequence identity with p5516, p00exe014, p004Ke009, p00jc, p00ke, or p00c0e030. In some embodiments, the cocktail comprises a fifth bacteriophage comprising at least 70%, 75%, 80%, 85%, 90%, 95%, or 100% sequence identity with p5516, p00exe014, p004Ke009, p00jc, p00ke, or p00c0e030. In some embodiments, the cocktail comprises a sixth bacteriophage comprising at least 70%, 75%, 80%, 85%, 90%, 95%, or 100% sequence identity with p5516, p00exe014, p004Ke009, p00jc, p00ke, or p00c0e030. In some embodiments, the cocktail comprises a seventh bacteriophage comprising at least 70%, 75%, 80%, 85%, 90%, 95%, or 100% sequence identity with p5516, p00exe014, p004Ke009, p00jc, p00ke, or p00c0e030.

In some embodiments, the cocktail comprises at least two bacteriophage, wherein the bacteriophage comprise p00ex, p004k, p00ke, p00jc, p5516, p6984, or p6977. In some embodiments, the first bacteriophage comprises at least 70%, 75%, 80%, 85%, 90% 95%, or 100% sequence identity with p00ex. In some embodiments, the cocktail comprises a second bacteriophage, wherein the second bacteriophage comprises at least 70%, 75%, 80%, 85%, 90% 95%, or 100% with p004k. In some embodiments, the cocktail comprises a third bacteriophage, wherein the third bacteriophage comprises at least 70%, 75%, 80%, 85%, 90% 95%, or 100% sequence identity with p00ke. In some embodiments, the cocktail comprises a fourth bacteriophage, wherein the fourth bacteriophage comprises at least 70%, 75%, 80%, 85%, 90% 95%, or 100% identity with p00jc. In some embodiments, the cocktail comprises a fifth bacteriophage, wherein the fifth bacteriophage comprises at least 70%, 75%, 80%, 85%, 90% 95%, or 100% sequence identity p5516. In some embodiments, the cocktail comprises a sixth bacteriophage, wherein the sixth bacteriophage comprises at least 70%, 75%, 80%, 85%, 90% 95%, or 100% sequence identity with p6984. In some embodiments, the cocktail comprises a seventh bacteriophage, wherein the seventh bacteriophage comprises at least 70%, 75%, 80%, 85%, 90% 95%, or 100% sequence identity with p6977. In some embodiments, at least one, two, three, four, or five bacteriophage comprise a CRISPR-Cas system. In some embodiments, at least one, two, or three bacteriophage do not comprise a CRISPR Cas system. In some embodiments, at least one, two, three, four, or five bacteriophage comprise a nucleic acid encoding a colicin. In some embodiments, at least one, two, three, four, or five bacteriophage comprise a nucleic acid encoding a colicin. In some embodiments, the colicin comprises Colicin U, Colicin K, Colicin 10, Colicin Ib, or a homolog thereof.

In some embodiments, described herein is a cocktail comprising at least two recombinant bacteriophage, wherein the first recombinant bacteriophage comprises at least 70%, 75%, 80%, 85%, 90% 95%, or 100% sequence identity with p00exe299. In some embodiments, the first bacteriophage comprises a nucleic sequence encoding a colicin. In some embodiments, the first bacteriophage comprises a sequence encoding Colicin U, Colicin K, Colicin 10, Colicin Ib, or homologs thereof. In some embodiments, the first bacteriophage comprises a sequence encoding Colicin U. In some embodiments, the first bacteriophage comprises a sequence encoding at least 70%, 75%, 80%, 85%, 90% 95%, or 100% sequence identity with SEQ ID NO 168. In some embodiments, the cocktail comprises a second bacteriophage comprising at least 70%, 75%, 80%, 85%, 90% 95%, or 100% sequence identity with p004ke127, p00jce098, p00kee072, p00c0e030, p00c0e103, p6977, or p6984. In some embodiments, the second bacteriophage comprises a sequence encoding Colicin U, Colicin K, Colicin 10, Colicin Ib, or homologs thereof. In some embodiments, the second bacteriophage comprises a sequence encoding at least 70%, 75%, 80%, 85%, 90% 95%, or 100% sequence identity with SEQ ID NO 165-168.

In some embodiments, described herein is a cocktail comprising at least two recombinant bacteriophage, wherein the first recombinant bacteriophage comprises at least 70%, 75%, 80%, 85%, 90% 95%, or 100% sequence identity with p004ke127. In some embodiments, the first bacteriophage comprises a nucleic sequence encoding a colicin. In some embodiments, the first bacteriophage comprises a sequence encoding Colicin U, Colicin K, Colicin 10, Colicin Ib, or homologs thereof. In some embodiments, the first bacteriophage comprises a sequence encoding Colicin K. In some embodiments, the first bacteriophage comprises a sequence encoding at least 70%, 75%, 80%, 85%, 90% 95%, or 100% sequence identity with SEQ ID NO 167. In some embodiments, the second bacteriophage comprises at least 70%, 75%, 80%, 85%, 90% 95%, or 100% sequence identity with p00exe299, p00jce098, p00kee072, p00c0e030, p00c0e103, p6977, or p6984. In some embodiments, the second bacteriophage comprises a sequence encoding Colicin U, Colicin K, Colicin 10, Colicin Ib, or homologs thereof. In some embodiments, the second bacteriophage comprises a sequence encoding at least 70%, 75%, 80%, 85%, 90% 95%, or 100% sequence identity with SEQ ID NO 165-168.

In some embodiments, described herein is a first recombinant bacteriophage comprising at least 70%, 75%, 80%, 85%, 90% 95%, or 100% sequence identity with p00jce098. In some embodiments, the first bacteriophage comprises a nucleic sequence encoding a colicin. In some embodiments, the first bacteriophage comprises a sequence encoding Colicin U, Colicin K, Colicin 10, Colicin Ib, or homologs thereof. In some embodiments, the first bacteriophage comprises a sequence encoding Colicin 10. In some embodiments, the first bacteriophage comprises a sequence encoding at least 70%, 75%, 80%, 85%, 90% 95%, or 100% sequence identity with SEQ ID NO 166. In some embodiments, the second bacteriophage comprises at least 70%, 75%, 80%, 85%, 90% 95%, or 100% sequence identity with p00exe299, p004ke127, p00kee072, p00c0e030, p00c0e103, p6977, or p6984. In some embodiments, the second bacteriophage comprises a sequence encoding Colicin U, Colicin K, Colicin 10, Colicin Ib, or homologs thereof. In some embodiments, the second bacteriophage comprises a sequence encoding at least 70%, 75%, 80%, 85%, 90% 95%, or 100% sequence identity with SEQ ID NO 165-168.

In some embodiments, described herein is a cocktail comprising a first recombinant bacteriophage comprising at least 70%, 75%, 80%, 85%, 90% 95%, or 100% sequence identity with p00kee072. In some embodiments, the first bacteriophage comprises a nucleic sequence encoding a colicin. In some embodiments, the first bacteriophage comprises a sequence encoding Colicin U, Colicin K, Colicin 10, Colicin Ib, or homologs thereof. In some embodiments, the first bacteriophage comprises a sequence encoding Colicin 10. In some embodiments, the first bacteriophage comprises a sequence encoding at least 70%, 75%, 80%, 85%, 90% 95%, or 100% sequence identity with SEQ ID NO 166. In some embodiments, the second bacteriophage comprises at least 70%, 75%, 80%, 85%, 90% 95%, or 100% sequence identity with p00exe299, p004ke127, p00jce098, p00c0e030, p00c0e103, p6977, or p6984. In some embodiments, the second bacteriophage comprises a sequence encoding Colicin U, Colicin K, Colicin 10, Colicin Ib, or homologs thereof. In some embodiments, the second bacteriophage comprises a sequence encoding at least 70%, 75%, 80%, 85%, 90% 95%, or 100% sequence identity with SEQ ID NO 165-168.

In some embodiments, described herein is a first recombinant bacteriophage comprising at least 70%, 75%, 80%, 85%, 90% 95%, or 100% sequence identity with p00c0e030. In some embodiments, the first recombinant bacteriophage comprising at least 70%, 75%, 80%, 85%, 90% 95%, or 100% sequence identity with p00c0e103. In some embodiments, the first bacteriophage comprises a nucleic sequence encoding a colicin. In some embodiments, the first bacteriophage comprises a sequence encoding Colicin U, Colicin K, Colicin 10, Colicin Ib, or homologs thereof. In some embodiments, the first bacteriophage comprises a sequence encoding at least 70%, 75%, 80%, 85%, 90% 95%, or 100% sequence identity with SEQ ID NO 164-168. In some embodiments, the second bacteriophage comprises at least 70%, 75%, 80%, 85%, 90% 95%, or 100% sequence identity with p00exe299, p004ke127, p00jce098, p00kee072, p6977, or p6984. In some embodiments, the second bacteriophage comprises a sequence encoding Colicin U, Colicin K, Colicin 10, Colicin Ib, or homologs thereof. In some embodiments, the second bacteriophage comprises a sequence encoding at least 70%, 75%, 80%, 85%, 90% 95%, or 100% sequence identity with SEQ ID NO 165-168.

In some embodiments, described herein is a cocktail comprising a first recombinant bacteriophage comprising at least 70%, 75%, 80%, 85%, 90% 95%, or 100% sequence identity with p6977. In some embodiments, the first bacteriophage comprises a nucleic sequence encoding a colicin. In some embodiments, the first bacteriophage comprises a sequence encoding Colicin U, Colicin K, Colicin 10, Colicin Ib, or homologs thereof. In some embodiments, the first bacteriophage comprises a sequence encoding at least 70%, 75%, 80%, 85%, 90% 95%, or 100% sequence identity with SEQ ID NO 164-168. In some embodiments, the second bacteriophage comprises at least 70%, 75%, 80%, 85%, 90% 95%, or 100% sequence identity with p00exe299, p004ke127, p00jce098, p00kee072, p00c0e030, p00c0e103, or p6984. In some embodiments, the second bacteriophage comprises a sequence encoding Colicin U, Colicin K, Colicin 10, Colicin Ib, or homologs thereof. In some embodiments, the second bacteriophage comprises a sequence encoding at least 70%, 75%, 80%, 85%, 90% 95%, or 100% sequence identity with SEQ ID NO 165-168.

In some embodiments, described herein is a cocktail comprising a first recombinant bacteriophage comprising at least 70%, 75%, 80%, 85%, 90% 95%, or 100% sequence identity with p6984. In some embodiments, the first bacteriophage comprises a nucleic sequence encoding a colicin. In some embodiments, the first bacteriophage comprises a sequence encoding Colicin U, Colicin K, Colicin 10, Colicin Ib, or homologs thereof. In some embodiments, the first bacteriophage comprises a sequence encoding at least 70%, 75%, 80%, 85%, 90% 95%, or 100% sequence identity with SEQ ID NO 164-168. In some embodiments, the second bacteriophage comprises at least 70%, 75%, 80%, 85%, 90% 95%, or 100% sequence identity with p00exe299, p004ke127, p00jce098, p00kee072, p00c0e030, p00coe103, or p6977. In some embodiments, the second bacteriophage comprises a sequence encoding Colicin U, Colicin K, Colicin 10, Colicin Ib, or homologs thereof. In some embodiments, the second bacteriophage comprises a sequence encoding at least 70%, 75%, 80%, 85%, 90% 95%, or 100% sequence identity with SEQ ID NO 165-168.

In some embodiments, the cocktail comprises at least two bacteriophage, wherein the bacteriophage comprise p004K, p00c0, p00ex, p00jc, p00ke, or p5516. In some embodiments, the cocktail comprises a first bacteriophage comprising at least 70%, 75%, 80%, 85%, 90%, 95%, or 100% sequence identity with p004ke009. In some embodiments, the cocktail comprises a second bacteriophage comprising at least 70%, 75%, 80%, 85%, 90%, 95%, or 100% sequence identity with p00c0. In some embodiments, the cocktail comprises a third bacteriophage comprising at least 70%, 75%, 80%, 85%, 90%, 95%, or 100% sequence identity with p00exe014. In some embodiments, the cocktail comprises a fourth bacteriophage comprising at least 70%, 75%, 80%, 85%, 90%, 95%, or 100% sequence identity with p00exe014. In some embodiments, the cocktail comprises a fifth bacteriophage comprising at least 70%, 75%, 80%, 85%, 90%, 95%, or 100% sequence identity with p00jc. In some embodiments, the cocktail comprises a fifth bacteriophage comprising at least 70%, 75%, 80%, 85%, 90%, 95%, or 100% sequence identity with p00jc, wherein the bacteriophage comprises a CRISPR-Cas system. In some embodiments, the cocktail comprises a fifth bacteriophage comprising at least 70%, 75%, 80%, 85%, 90%, 95%, or 100% sequence identity with p00ke. In some embodiments, the cocktail comprises a fifth bacteriophage comprising at least 70%, 75%, 80%, 85%, 90%, 95%, or 100% sequence identity with p00ke, wherein the bacteriophage comprises a CRISPR-Cas system. In some embodiments, the cocktail comprises a sixth bacteriophage comprising at least 70%, 75%, 80%, 85%, 90%, 95%, or 100% sequence identity with p5516.

In some embodiments, the first bacteriophage comprises at least 70%, 75%, 80%, 85%, 90% 95%, or 100% sequence identity with p004K. In some embodiments, the cocktail comprises a second bacteriophage, wherein the second bacteriophage comprises at least 70%, 75%, 80%, 85%, 90% 95%, or 100% with p00c0, p00ex, p00jc, p00ke, or p5516. In some embodiments, the cocktail comprises a third bacteriophage, wherein the third bacteriophage comprises at least 70%, 75%, 80%, 85%, 90% 95%, or 100% with p00c0, p00ex, p00jc, p00ke, or p5516. In some embodiments, the cocktail comprises a fourth bacteriophage, wherein the fourth bacteriophage comprises at least 70%, 75%, 80%, 85%, 90% 95%, or 100% with p00c0, p00ex, p00jc, p00ke, or p5516. In some embodiments, the cocktail comprises a fifth bacteriophage, wherein the fifth bacteriophage comprises at least 70%, 75%, 80%, 85%, 90% 95%, or 100% with p00c0, p00ex, p00jc, p00ke, or p5516. In some embodiments, the cocktail comprises a sixth bacteriophage, wherein the sixth bacteriophage comprises at least 70%, 75%, 80%, 85%, 90% 95%, or 100% with p00c0, p00ex, p00jc, p00ke, or p5516. In some embodiments, at least one, two, three, four, or five bacteriophage comprise a CRISPR-Cas system. In some embodiments, at least one, two, or three bacteriophage do not comprise a CRISPR Cas system.

In some embodiments, the first bacteriophage comprises at least 70%, 75%, 80%, 85%, 90% 95%, or 100% sequence identity with p00c0. In some embodiments, the cocktail comprises a second bacteriophage, wherein the second bacteriophage comprises at least 70%, 75%, 80%, 85%, 90% 95%, or 100% with p004K, p00ex, p00jc, p00ke, or p5516. In some embodiments, the cocktail comprises a third bacteriophage, wherein the third bacteriophage comprises at least 70%, 75%, 80%, 85%, 90% 95%, or 100% with p004K, p00ex, p00jc, p00ke, or p5516. In some embodiments, the cocktail comprises a fourth bacteriophage, wherein the fourth bacteriophage comprises at least 70%, 75%, 80%, 85%, 90% 95%, or 100% with p004K, p00ex, p00jc, p00ke, or p5516. In some embodiments, the cocktail comprises a fifth bacteriophage, wherein the fifth bacteriophage comprises at least 70%, 75%, 80%, 85%, 90% 95%, or 100% with p004K, p00ex, p00jc, p00ke, or p5516. In some embodiments, the cocktail comprises a sixth bacteriophage, wherein the sixth bacteriophage comprises at least 70%, 75%, 80%, 85%, 90% 95%, or 100% with p004K, p00ex, p00jc, p00ke, or p5516. In some embodiments, at least one, two, three, four, or five bacteriophage comprise a CRISPR-Cas system. In some embodiments, at least one, two, or three bacteriophage do not comprise a CRISPR Cas system.

In some embodiments, the first bacteriophage comprises at least 70%, 75%, 80%, 85%, 90% 95%, or 100% sequence identity with p00ex. In some embodiments, the cocktail comprises a second bacteriophage, wherein the second bacteriophage comprises at least 70%, 75%, 80%, 85%, 90% 95%, or 100% with p00c0, p004K, p00jc, p00ke, or p5516. In some embodiments, the cocktail comprises a third bacteriophage, wherein the third bacteriophage comprises at least 70%, 75%, 80%, 85%, 90% 95%, or 100% with p00c0, p004K, p00jc, p00ke, or p5516. In some embodiments, the cocktail comprises a fourth bacteriophage, wherein the fourth bacteriophage comprises at least 70%, 75%, 80%, 85%, 90% 95%, or 100% with p00c0, p004K, p00jc, p00ke, or p5516. In some embodiments, the cocktail comprises a fifth bacteriophage, wherein the fifth bacteriophage comprises at least 70%, 75%, 80%, 85%, 90% 95%, or 100% with p00c0, p004K, p00jc, p00ke, or p5516. In some embodiments, the cocktail comprises a sixth bacteriophage, wherein the sixth bacteriophage comprises at least 70%, 75%, 80%, 85%, 90% 95%, or 100% with p00c0, p004K, p00jc, p00ke, or p5516. In some embodiments, at least one, two, three, four, or five bacteriophage comprise a CRISPR-Cas system. In some embodiments, at least one, two, or three bacteriophage do not comprise a CRISPR Cas system.

In some embodiments, the first bacteriophage comprises at least 70%, 75%, 80%, 85%, 90% 95%, or 100% sequence identity with p00jc. In some embodiments, the cocktail comprises a second bacteriophage, wherein the second bacteriophage comprises at least 70%, 75%, 80%, 85%, 90% 95%, or 100% with p00c0, p00ex, p004K, p00ke, or p5516. In some embodiments, the cocktail comprises a third bacteriophage, wherein the third bacteriophage comprises at least 70%, 75%, 80%, 85%, 90% 95%, or 100% with p00c0, p00ex, p004K, p00ke, or p5516. In some embodiments, the cocktail comprises a fourth bacteriophage, wherein the fourth bacteriophage comprises at least 70%, 75%, 80%, 85%, 90% 95%, or 100% with p00c0, p00ex, p004K, p00ke, or p5516. In some embodiments, the cocktail comprises a fifth bacteriophage, wherein the fifth bacteriophage comprises at least 70%, 75%, 80%, 85%, 90% 95%, or 100% with p00c0, p00ex, p004K, p00ke, or p5516. In some embodiments, the cocktail comprises a sixth bacteriophage, wherein the sixth bacteriophage comprises at least 70%, 75%, 80%, 85%, 90% 95%, or 100% with p00c0, p00ex, p004K, p00ke, or p5516. In some embodiments, at least one, two, three, four, or five bacteriophage comprise a CRISPR-Cas system. In some embodiments, at least one, two, or three bacteriophage do not comprise a CRISPR Cas system.

In some embodiments, the first bacteriophage comprises at least 70%, 75%, 80%, 85%, 90% 95%, or 100% sequence identity with p00ke. In some embodiments, the cocktail comprises a second bacteriophage, wherein the second bacteriophage comprises at least 70%, 75%, 80%, 85%, 90% 95%, or 100% with p00c0, p00ex, p00jc, p004K, or p5516. In some embodiments, the cocktail comprises a third bacteriophage, wherein the third bacteriophage comprises at least 70%, 75%, 80%, 85%, 90% 95%, or 100% with p00c0, p00ex, p00jc, p004K, or p5516. In some embodiments, the cocktail comprises a fourth bacteriophage, wherein the fourth bacteriophage comprises at least 70%, 75%, 80%, 85%, 90% 95%, or 100% with p00c0, p00ex, p00jc, p004K, or p5516. In some embodiments, the cocktail comprises a fifth bacteriophage, wherein the fifth bacteriophage comprises at least 70%, 75%, 80%, 85%, 90% 95%, or 100% with p00c0, p00ex, p00jc, p004K, or p5516. In some embodiments, the cocktail comprises a sixth bacteriophage, wherein the sixth bacteriophage comprises at least 70%, 75%, 80%, 85%, 90% 95%, or 100% with p00c0, p00ex, p00jc, p004K, or p5516. In some embodiments, at least one, two, three, four, or five bacteriophage comprise a CRISPR-Cas system. In some embodiments, at least one, two, or three bacteriophage do not comprise a CRISPR Cas system.

In some embodiments, the first bacteriophage comprises at least 70%, 75%, 80%, 85%, 90% 95%, or 100% sequence identity with p5516. In some embodiments, the cocktail comprises a second bacteriophage, wherein the second bacteriophage comprises at least 70%, 75%, 80%, 85%, 90% 95%, or 100% with p00c0, p00ex, p00jc, p00ke, or p004K. In some embodiments, the cocktail comprises a third bacteriophage, wherein the third bacteriophage comprises at least 70%, 75%, 80%, 85%, 90% 95%, or 100% with p00c0, p00ex, p00jc, p00ke, or p004K. In some embodiments, the cocktail comprises a fourth bacteriophage, wherein the fourth bacteriophage comprises at least 70%, 75%, 80%, 85%, 90% 95%, or 100% with p00c0, p00ex, p00jc, p00ke, or p004K. In some embodiments, the cocktail comprises a fifth bacteriophage, wherein the fifth bacteriophage comprises at least 70%, 75%, 80%, 85%, 90% 95%, or 100% with p00c0, p00ex, p00jc, p00ke, or p004K. In some embodiments, the cocktail comprises a sixth bacteriophage, wherein the sixth bacteriophage comprises at least 70%, 75%, 80%, 85%, 90% 95%, or 100% with p00c0, p00ex, p00jc, p00ke, or p004K. In some embodiments, at least one, two, three, four, or five bacteriophage comprise a CRISPR-Cas system. In some embodiments, at least one, two, or three bacteriophage do not comprise a CRISPR Cas system.

In some embodiments, bacteriophages of interest are obtained from environmental sources or from commercial research vendors. In some embodiments, obtained bacteriophages are screened for lytic activity against a library of bacteria and their associated strains. In some embodiments, the bacteriophages are screened against a library of bacteria and their associated strains for their ability to generate primary resistance in the screened bacteria.

### Insertion Sites

In some embodiments, the insertion of the nucleic acid sequence into a bacteriophage preserves the lytic activity of the bacteriophage. In some embodiments, the nucleic acid sequence is inserted into the bacteriophage genome. In some embodiments, the nucleic acid sequence is inserted into the bacteriophage genome at a transcription terminator site at the end of an operon of interest. In some embodiments, the nucleic acid sequence is inserted into the bacteriophage genome as a replacement for one or more removed non-essential genes. In some embodiments, the nucleic acid sequence is inserted into the bacteriophage genome as a replacement for one or more removed lysogenic genes. In some embodiments, the replacement of non-essential and/or lysogenic genes with the nucleic acid sequence does not affect the lytic activity of the bacteriophage. In some embodiments, the replacement of non-essential and/or lysogenic genes with the nucleic acid sequence preserves the lytic activity of the bacteriophage. In some embodiments, the replacement of non-essential and/or lysogenic genes with the nucleic acid sequence enhances the lytic activity of the bacteriophage. In some embodiments, the replacement of non-essential and/or lysogenic genes with the nucleic acid sequence renders a lysogenic bacteriophage lytic.

In some embodiments, the nucleic acid sequence is introduced into the bacteriophage genome at a first location while one or more non-essential and/or lysogenic genes are separately removed and/or inactivated from the bacteriophage genome at a separate location. In some embodiments, the nucleic acid sequence is introduced into the bacteriophage at a first location while one or more non-essential and/or lysogenic genes are separately removed and/or inactivated from the bacteriophage genome at multiple separate locations. In some embodiments, the removal and/or inactivation of one or more non-essential and/or lysogenic genes does not affect the lytic activity of the bacteriophage. In some embodiments, the removal and/or inactivation of one or more non-essential and/or lysogenic genes preserves the lytic activity of the bacteriophage. In some embodiments, the removal of one or more non-essential and/or lysogenic genes renders a lysogenic bacteriophage into a lytic bacteriophage.

In some embodiments, the bacteriophage is a temperate bacteriophage which has been rendered lytic by any of the aforementioned means. In some embodiments, a temperate bacteriophage is rendered lytic by the removal, replacement, or inactivation of one or more lysogenic genes. In some embodiments, the lytic activity of the bacteriophage is due to the removal, replacement, or inactivation of at least one lysogeny gene. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the lysogenic gene is an activator gene. In some embodiments, the lysogenic gene is cII gene. In some embodiments, the lysogenic gene is lexA gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, a temperate bacteriophage is rendered lytic by the insertion of one or more lytic genes. In some embodiments, a temperate bacteriophage is rendered lytic by the insertion of one or more genes that contribute to the induction of a lytic cycle. In some embodiments, a temperate bacteriophage is rendered lytic by altering the expression of one or more genes that contribute to the induction of a lytic cycle. In some embodiments, a temperate bacteriophage phenotypically changes from a lysogenic bacteriophage to a lytic bacteriophage. In some embodiments, a temperate bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI). In some embodiments, a temperate bacteriophage that is rendered lytic is prevented from reverting to lysogenic state. In some embodiments, a temperate bacteriophage that is rendered lytic is prevented from reverting back to lysogenic state by way the self-targeting activity of the first introduced CRISPR array. In some embodiments, a temperate bacteriophage that is rendered lytic is prevented from reverting back to lysogenic state by way of introducing an additional CRISPR array. In some embodiments, the bacteriophage does not confer any new properties onto the target bacterium beyond cellular death cause by lytic activity of the bacteriophage and/or the activity of the first or second CRISPR array.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

### Non-Essential Gene

In some embodiments, the non-essential gene to be removed and/or replaced from the bacteriophage is a gene that is non-essential for the survival of the bacteriophage. In some embodiments, the non-essential gene to be removed and/or replaced from the bacteriophage is a gene that is non-essential for the induction and/or maintenance of lytic cycle.

### Transcriptional Activators

In some embodiments, the nucleic acid sequence further comprises a transcriptional activator. In some embodiments, the transcriptional activator encoded regulates the expression of genes of interest within the Escherichia species. In some embodiments, the transcriptional activator activates the expression of genes of interest within the Escherichia species whether exogenous or endogenous. In some embodiments, the transcriptional activator activates the expression genes of interest within the Escherichia species by disrupting the activity of one or more inhibitory elements within the Escherichia species. In some embodiments, the inhibitory element comprises a transcriptional repressor. In some embodiments, the inhibitory element comprises a global transcriptional repressor. In some embodiments the inhibitory element is a histone-like nucleoid-structuring (H-NS) protein or homologue or functional fragment thereof. In some embodiments, the inhibitory element is a leucine responsive regulatory protein (LRP). In some embodiments, the inhibitory element is a CodY protein.

In some bacteria, the CRISPR-Cas system is poorly expressed and considered silent under most environmental conditions. In these bacteria, the regulation of the CRISPR-Cas system is the result of the activity of transcriptional regulators, for example histone-like nucleoid-structuring (H-NS) protein which is widely involved in transcriptional regulation of the host genome. H-NS exerts control over host transcriptional regulation by multimerization along AT-rich sites resulting in DNA bending. In some bacteria, such as E. coli, the regulation of the CRISPR-Cas3 operon is regulated by H-NS.

Similarly, in some bacteria, the repression of the CRISPR-Cas system is controlled by an inhibitory element, for example the leucine responsive regulatory protein (LRP). LRP has been implicated in binding to upstream and downstream regions of the transcriptional start sites. Notably, the activity of LRP in regulating expression of the CRISPR-Cas system varies from bacteria to bacteria. Unlike, H-NS which has broad inter-species repression activity, LRP has been shown to differentially regulate the expression of the host CRISPR-Cas system. As such, in some instances, LRP reflects a host-specific means of regulating CRISPR-Cas system expression in different bacteria.

In some instances, the repression of CRISPR-Cas system is also controlled by inhibitory element CodY. CodY is a GTP-sensing transcriptional repressor that acts through DNA binding. The intracellular concentration of GTP acts as an indicator for the environmental nutritional status. Under normal culture conditions, GTP is abundant and binds with CodY to repress transcriptional activity. However, as GTP concentrations decreases, CodY becomes less active in binding DNA, thereby allowing transcription of the formerly repressed genes to occur. As such, CodY acts as a stringent global transcriptional repressor.

In some embodiments, the transcriptional activator is a LeuO polypeptide, homolog or functional fragment thereof, a leuO coding sequence, or an agent that upregulates LeuO. In some embodiments, the transcriptional activator comprises any ortholog or functional equivalent of LeuO. In some bacteria, LeuO acts in opposition to H-NS by acting as a global transcriptional regulator that responds to environmental nutritional status of a bacterium. Under normal conditions, LeuO is poorly expressed. However, under amino acid starvation and/or reaching of the stationary phase in the bacterial life cycle, LeuO is upregulated. Increased expression of LeuO leads to it antagonizing H-NS at overlapping promoter regions to effect gene expression. Overexpression of LeuO upregulates the expression of the CRISPR-Cas system.

In some embodiments, the expression of LeuO leads to disruption of an inhibitory element. In some embodiments, the disruption of an inhibitory element due to expression of LeuO removes the transcriptional repression of a CRISPR-Cas system. In some embodiments, the expression of LeuO removes transcriptional repression of a CRISPR-Cas system due to activity of H-NS. In some embodiments, the disruption of an inhibitory element due to the expression of LeuO causes an increase in the expression of a CRISPR-Cas system. In some embodiments, the increase in the expression of a CRISPR-Cas system due to the disruption of an inhibitory element caused by the expression of LeuO causes an increase in the CRISPR-Cas processing of a nucleic acid sequence comprising a CRISPR array. In some embodiments, the increase in the expression of a CRISPR-Cas system due to the disruption of an inhibitory element by the expression of LeuO causes an increase in the CRISPR-Cas processing of a nucleic acid sequence comprising a CRISPR array so as to increase the level of lethality of the CRISPR array against a bacterium. In some embodiments, transcriptional activator causes increase activity of a bacteriophage and/or the CRISPR-Cas system.

### Regulatory Elements

In some embodiments, the nucleic acid sequences are operatively associated with a variety of promoters, terminators and other regulatory elements for expression in various organisms or cells. In some embodiments, the nucleic acid sequence further comprises a leader sequence. In some embodiments, the nucleic acid sequence further comprises a promoter sequence. In some embodiments, at least one promoter and/or terminator is operably linked to the nucleic acid encoding the colicin or the CRISPR array. Any promoter useful with this disclosure is used and includes, for example, promoters functional with the organism of interest as well as constitutive, inducible, developmental regulated, tissue-specific/preferred-promoters, and the like, as disclosed herein. A regulatory element as used herein is endogenous or heterologous. In some embodiments, an endogenous regulatory element derived from the subject organism is inserted into a genetic context in which it does not naturally occur (e.g. a different position in the genome than as found in nature), thereby producing a recombinant or non-native nucleic acid.

In some embodiments, expression of the nucleic acid sequence is constitutive, inducible, temporally regulated, developmentally regulated, or chemically regulated. In some embodiments, the expression of the nucleic acid sequence is made constitutive, inducible, temporally regulated, developmentally regulated, or chemically regulated by operatively linking the nucleic acid sequence to a promoter functional in an organism of interest. In some embodiments, repression is made reversible by operatively linking the nucleic acid sequence to an inducible promoter that is functional in an organism of interest. The choice of promoter disclosed herein varies depending on the quantitative, temporal and spatial requirements for expression, and also depending on the host cell to be transformed.

Exemplary promoters for use with the methods, bacteriophages and compositions disclosed herein include promoters that are functional in bacteria. For example, L-arabinose inducible (araBAD, P_{BAD}) promoter, any lac promoter, L-rhamnose inducible (rhaPBAD) promoter, T7 RNA polymerase promoter, trc promoter, tac promoter, lambda bacteriophage promoter (p_{L}p_{L}-9G-50), anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, a-amylase (Pamy), Ptms, P43 (comprised of two overlapping RNA polymerase a factor recognition sites, σ, σB), Ptms, P43, rplK-rplA, ferredoxin promoter, and/or xylose promoter. In some embodiments, the promoter is a BBa_J23102 promoter. In some embodiments, the promoter works in a broad range of bacteria, such as BBa_J23104, BBa_J23109, Bba_J23102. In some embodiments the promoter is derived from the target bacterium, such as endogenous CRISPR promoter, endogenous Cas operon promoter, p16, plpp, or ptat. In some embodiments, the promoter is a bacteriophage promoter, such as the promoter for gp105 or gp245.

In some embodiments, the promoter comprises at least or about 70%, 80%, 85%, 90%, 91%, 92%, 93%, 94%, 95%, 96%, 97%, 98%, 99%, or 100% sequence identity to any one of SEQ ID NOs: 1-11, 19, 64-65 or 70-160. In some instances, the promoter comprises at least or about 95% sequence identity to any one of SEQ ID NOS: 1-11, 19, 64-65 or 70-160. In some instances, the promoter comprises at least or about 97% sequence identity to any one of SEQ ID NOS: 1-11, 19, 64-65 or 70-160. In some instances, the promoter comprises at least or about 99% sequence identity to any one of SEQ ID NOS: 1-11, 19, 64-65 or 70-160. In some instances, the promoter comprises 100% sequence identity to any one of SEQ ID NOS: 1-11, 19, 64-65 or 70-160. In some instances, the promoter comprises at least a portion having at least or about 3, 4, 5, 6, 7, 8, 9, 10, 12, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more than 50 nucleotides of any one of SEQ ID NOS: 1-11, 19, 64-65 or 70-160. In some instances, the promoter comprises at least a portion having at least or about 50, 55, 60, 65, 70, 75, 80, 85, 90, 95, 100, 105, 110, 115 120, 125, 130, 135, 140, 145, 150, 155, 160, 165, 170, 175, 180, 185, 190, 195, 200, 205, 210, 215, or more than 215 nucleotides of any one of SEQ ID NOS: 1-11, 19, 64-65 or 70-160. In some embodiments, the promoter comprises 10-300, 20-300, 30-300, 40-300, 50-300, 60-300, 70-300, 80-300, 90-300, 100-300, 110-300, 120-300, 130-300, 140-300, 150-300, 160-300, 170-300, 180-300, 190-300, 210-300, 220-300, 230-300, 240-300, 250-300, 260-300, 270-300, 280-300, 290-300 nucleotides with sequence identity to any one of SEQ ID NOS: 1-11, 19, 64-65 or 70-160.

In some embodiments, inducible promoters are used. In some embodiments, chemical-regulated promoters are used to modulate the expression of a gene in an organism through the application of an exogenous chemical regulator. The use of chemically regulated promoters enables RNAs and/or the polypeptides encoded by the nucleic acid sequence to be synthesized only when, for example, an organism is treated with the inducing chemicals. In some embodiments where a chemical-inducible promoter is used, the application of a chemical induces gene expression. In some embodiments wherein a chemical-repressible promoter is used, the application of the chemical represses gene expression. In some embodiments, the promoter is a light-inducible promoter, where application of specific wavelengths of light induces gene expression. In some embodiments, a promoter is a light-repressible promoter, where application of specific wavelengths of light represses gene expression.

### Expression Cassette

In some embodiments, the nucleic acid sequence is an expression cassette or in an expression cassette. In some embodiments, the expression cassettes are designed to express the nucleic acid sequence disclosed herein. In some embodiments, the nucleic acid sequence is an expression cassette encoding a colicin. In some embodiments, the nucleic acid sequence is an expression cassette encoding components of a CRISPR-Cas system. In some embodiments, the nucleic acid sequence is an expression cassette encoding components of a Type I CRISPR-Cas system. In some embodiments, the nucleic acid sequence is an expression cassette encoding an operable CRISPR-Cas system. In some embodiments, the nucleic acid sequence is an expression cassette encoding the operable components of a Type I CRISPR-Cas system, including Cascade and Cas3. In some embodiments, the nucleic acid sequence is an expression cassette encoding the operable components of a Type I CRISPR-Cas system, including a crRNA, Cascade and Cas3.

In some embodiments, an expression cassette comprising a nucleic acid sequence of interest is chimeric, meaning that at least one of its components is heterologous with respect to at least one of its other components. In some embodiments, an expression cassette is naturally occurring but has been obtained in a recombinant form useful for heterologous expression.

In some embodiments, an expression cassette includes a transcriptional and/or translational termination region (i.e. termination region) that is functional in the selected host cell. In some embodiments, termination regions are responsible for the termination of transcription beyond the heterologous nucleic acid sequence of interest and for correct mRNA polyadenylation. In some embodiments, the termination region is native to the transcriptional initiation region, is native to the operably linked nucleic acid sequence of interest, is native to the host cell, or is derived from another source (i.e., foreign or heterologous to the promoter, to the nucleic acid sequence of interest, to the host, or any combination thereof). In some embodiments, terminators are operably linked to the nucleic acid sequence disclosed herein.

In some embodiments, an expression cassette includes a nucleotide sequence for a selectable marker. In some embodiments, the nucleotide sequence encodes either a selectable or a screenable marker, depending on whether the marker confers a trait that is selected for by chemical means, such as by using a selective agent (e.g. an antibiotic), or on whether the marker is simply a trait that one identifies through observation or testing, such as by screening (e.g., fluorescence).

### Vectors

In addition to expression cassettes, the nucleic acid sequences disclosed herein (e.g. nucleic acid sequence comprising a CRISPR array) are used in connection with vectors. A vector comprises a nucleic acid molecule comprising the nucleotide sequence(s) to be transferred, delivered or introduced. Non-limiting examples of general classes of vectors include, but are not limited to, a viral vector, a plasmid vector, a bacteriophage vector, a phagemid vector, a cosmid vector, a fosmid vector, a bacteriophage, an artificial chromosome, or an agrobacterium binary vector in double or single stranded linear or circular form which may or may not be self-transmissible or mobilizable. A vector transforms prokaryotic or eukaryotic host either by integration into the cellular genome or exist extrachromosomally (e.g. autonomous replicating plasmid with an origin of replication). Additionally, included are shuttle vectors by which is meant a DNA vehicle capable, naturally or by design, of replication in two different host organisms. In some embodiments, a shuttle vector replicates in actinomycetes and bacteria and/or eukaryotes. In some embodiments, the nucleic acid in the vector are under the control of, and operably linked to, an appropriate promoter or other regulatory elements for transcription in a host cell. In some embodiments, the vector is a bi-functional expression vector which functions in multiple hosts.

### Sequence Optimization

In some embodiments, the nucleic acid sequence encoding a payload (e.g. the nucleic acid insert) is optimized for stable expression in a bacteriophage genome. In some embodiments, the insert is stable through at least 2, 3, 4, 5, 6, 7, 8, 9, or 10 generations of passaging. In some embodiments, the nucleic acid sequence is optimized by optimizing the insertion site, modifying secondary structures, modifying DNA modification sites, modifying restriction enzyme motifs, codon optimization, GC % optimization, or a combination thereof. In some embodiments, the insertion site of the nucleic acid sequence is optimized. In some embodiments, the nucleic acid sequence is modified to remove secondary structures.

In certain embodiments, the bacteriophage comprises a nucleic acid insert modified from an exogenous nucleic acid described herein, wherein the nucleic acid comprises a first plurality of codons encoding for a first protein, and the nucleic acid insert comprises a second plurality of codons encoding for a second protein, wherein the first protein and the second protein have at least 90% amino acid sequence identity, and wherein at least 50% of the second plurality of codons are high frequency codons in the bacteriophage genome. In certain aspects, described herein is a method of inserting an exogenous sequence comprising a plurality of codons encoding a first protein into a bacteriophage, the method comprising substituting one or more of the plurality of codons with a codon native to the bacteriophage to generate a nucleic acid insert encoding a second protein, wherein the first protein and the second protein have at least 90% amino acid sequence identity. In certain aspects, described herein is a nucleic acid insert modified from an exogenous nucleic acid, wherein the nucleic acid comprises a first plurality of codons encoding for a first protein, and the nucleic acid insert comprises a second plurality of codons encoding for a second protein, wherein the first protein and the second protein have at least 90% amino acid sequence identity, and wherein at least 50% of the second plurality of codons are high frequency codons in the bacteriophage genome. In some embodiments, the first protein and the second protein have at least 95%, 97.5%, 99% or 99.5% sequence identity. In some embodiments, at least 50%<60%, 70%, 80%, 90% or more than 90% of the second plurality of codons are high frequency codons in the bacteriophage genome. In some embodiments, the second plurality of codons match the profile of codons in the bacteriophage genome.

In some embodiments, the nucleic acid sequence is modified to remove DNA modification sites. In some embodiments, the DNA modification sites comprise DNA methylation sites.

In some embodiments, the nucleic acid sequence is modified to remove restriction enzyme motifs. In some embodiments, the nucleic acid sequence is modified to remove restriction enzyme motifs for a restriction enzyme derived from a bacterial species described herein. In some embodiments, the nucleic acid insert does not comprise, or comprises fewer than 10 sites recognized by a bacterial enzyme.

In some embodiments, the nucleic acid sequence is codon optimized for expression in any species of interest. Codon optimization involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species-specific codon usage table with the codons present in the native polynucleotide sequences. Codon optimization of a nucleotide sequence results in a nucleotide sequence having less than 100% identity (e.g., 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function as that encoded by the original nucleotide sequence. In some embodiments, the nucleic acid sequences of this disclosure are codon optimized for expression in the organism/species of interest.

In some embodiments, the nucleic acid sequence is modified to optimize the percent GC content. In some embodiments, the percent GC content is modified so that at least 10%, 20%, 30%, 40%, 50%, 60%, 70%, 80% 90% or more than 90% of the nucleotides comprises guanine or cytosine. In some embodiments, the percent GC content is modified so that no more than 10%, 20%, 30%, 40%, 50%, 60%, 70%, 80% 90% or more than 90% of the nucleotides comprises guanine or cytosine.

In some embodiments, the exogenous nucleic acid is a bacterial nucleic acid. In some embodiments, the nucleic acid insert and the bacterial nucleic acid have less than 100%, 95%, 90%, 80%, 70%, 60%, or 50% sequence identity. In some embodiments, the first bacterial protein is a CRISPR-Cas protein as described herein. In some embodiments, the first bacterial protein is an antimicrobial agent and/or peptide as described herein.

### Transformation

In some embodiments, the nucleic acid sequence, and/or expression cassettes disclosed herein are expressed transiently and/or stably incorporated into the genome of a host organism. In some embodiments, a the nucleic acid sequence and/or expression cassettes disclosed herein is introduced into a cell by any method known to those of skill in the art. Exemplary methods of transformation include transformation via electroporation of competent cells, passive uptake by competent cells, chemical transformation of competent cells, as well as any other electrical, chemical, physical (mechanical) and/or biological mechanism that results in the introduction of nucleic acid into a cell, including any combination thereof. In some embodiments, transformation of a cell comprises nuclear transformation. In some embodiments, transformation of a cell comprises plasmid transformation and conjugation.

In some embodiments, when more than one nucleic acid sequence is introduced, the nucleotide sequences are assembled as part of a single nucleic acid construct, or as separate nucleic acid constructs, and are located on the same or different nucleic acid constructs. In some embodiments, nucleotide sequences are introduced into the cell of interest in a single transformation event, or in separate transformation events.

### Methods of Use

Disclosed herein, in certain embodiments, are methods of killing a E. coli comprising introducing into the E. coli any of the bacteriophages disclosed herein.

Further disclosed herein, in certain embodiments, are methods of modifying a mixed population of bacterial cells having a first bacterial species that comprises a target nucleotide sequence in the essential gene and a second bacterial species that does not comprise a target nucleotide sequence in the essential gene, the method comprising introducing into the mixed population of bacterial cells any of the bacteriophages disclosed herein.

Also disclosed herein, in certain embodiments, are methods of treating a disease in an individual in need thereof, the method comprising administering to the individual any of the bacteriophages disclosed herein.

In some embodiments, the population of bacteria is contacted with one or more colicins. In some embodiments, the one or more colicins comprises Colicin Ib, Colicin 10, Colicin U, Colicin K, or a combination thereof. In some embodiments, the population of bacteria is contacted with at least a first colicin and a second colicin, wherein the first colicin comprises Colicin Ib and the second colicin comprises Colicin 10, Colicin U, Colicin K. In some embodiments, the population of bacteria is contacted with at least a first colicin and a second colicin, wherein the first colicin comprises Colicin 10 and the second colicin comprises Colicin Ib, Colicin U, Colicin K. In some embodiments, the population of bacteria is contacted with at least a first colicin and a second colicin, wherein the first colicin comprises Colicin U and the second colicin comprises Colicin Ib, Colicin 10, Colicin K. In some embodiments, the population of bacteria is contacted with at least a first colicin and a second colicin, wherein the first colicin comprises Colicin K and the second colicin comprises Colicin Ib, Colicin 10, Colicin U.

In some embodiments, the E. coli is killed solely by lytic activity of the bacteriophage. In some embodiments, the E. coli is killed solely by the activity of the colicin. In some embodiments, the E. coli is killed solely by activity of the CRISPR-Cas system. In some embodiments, the E. coli is killed by the processing of the CRISPR array by a CRISPR-Cas system to produce a processed crRNA capable of directing CRISPR-Cas based endonuclease activity and/or cleavage at the target nucleotide sequence in the target gene of the bacterium.

In some embodiments, the E. coli is killed by lytic activity of the bacteriophage in combination with activity of the Type I CRISPR-Cas system. In some embodiments, the E. coli is killed by the activity of the Type I CRISPR-Cas system, independently of the lytic activity of the bacteriophage. In some embodiments, the activity of the Type I CRISPR-Cas system supplements or enhances the lytic activity of the bacteriophage. In some embodiments, the activity of the Type I CRISPR-Cas system and the lytic activity of the bacteriophage are additive.

In some embodiments, the E. coli is killed by lytic activity of the bacteriophage in combination with activity of the colicin. In some embodiments, the E. coli is killed by the activity of the colicin, independently of the lytic activity of the bacteriophage. In some embodiments, the activity of the colicin supplements or enhances the lytic activity of the bacteriophage. In some embodiments, the activity of the colicin and the lytic activity of the bacteriophage are additive.

In some embodiments, the lytic activity of the bacteriophage and the activity of the Type I CRISPR-Cas system is synergistic. In some embodiments, a synergistic activity is defined as an activity resulting in a greater level of bacteriophage kill than the additive combination of the lytic activity of the bacteriophage and the Type I CRISPR-Cas system. In some embodiments, the lytic activity of the bacteriophage is modulated by a concentration of the bacteriophage. In some embodiments, the activity of the Type I CRISPR-Cas system is modulated by a concentration of the bacteriophage.

In some embodiments, the lytic activity of the bacteriophage and the activity of the colicin is synergistic. In some embodiments, a synergistic activity is defined as an activity resulting in a greater level of bacteriophage kill than the additive combination of the lytic activity of the bacteriophage and the colicin. In some embodiments, the lytic activity of the bacteriophage is modulated by a concentration of the bacteriophage. In some embodiments, the activity of the colicin is modulated by a concentration of the bacteriophage.

In some embodiments, the synergistic killing of the bacterium is modulated to favor killing by the lytic activity of the bacteriophage over the activity of the first CRISPR-Cas system by increasing the concentration of bacteriophage administered to the bacterium. In some embodiments, the synergistic killing of the bacterium is modulated to disfavor killing by the lytic activity of the bacteriophage over the activity of the CRISPR-Cas system by decreasing the concentration of bacteriophage administered to the bacterium. In some embodiments, at low concentrations, lytic replication allows for amplification and killing of the target bacteria. In some embodiments, at high concentrations, amplification of a bacteriophage is not required. In some embodiments, the synergistic killing of the bacterium is modulated to favor killing by the activity of the CRISPR-Cas system over the lytic activity of the bacteriophage by altering the number, the length, the composition, the identity, or any combination thereof, of the spacers so as to increase the lethality of the CRISPR array. In some embodiments, the synergistic killing of the bacterium is modulated to disfavor killing by the activity of the CRISPR-Cas system over the lytic activity of the bacteriophage by altering the number, the length, the composition, the identity, or any combination thereof, of the spacers so as to decrease the lethality of the CRISPR array.

### Administration Routes and Dosage

Dose and duration of the administration of a composition disclosed herein will depend on a variety of factors, including the subject's age, subject's weight, and tolerance of the bacteriophage. In some embodiments, a bacteriophage disclosed herein is administered to patients intra-arterially, intravenously, intraurethrally, intramuscularly, orally, subcutaneously, by inhalation, or any combination thereof. In some embodiments, a bacteriophage disclosed herein is administered to patients by oral administration. In some embodiments, a bacteriophage disclosed herein is administered to patients by topical, cutaneous, transdermal, transmucosal, implantation, sublingual, buccal, rectal, vaginal, ocular, otic, or nasal administration. In some embodiments, a bacteriophage disclosed herein is administered to patients by any combination of the aforementioned routes of administration.

In some embodiments, the bacteriophage or bacteriophage cocktail is administered intravenously for at least 1, 2, 3, 4, 5, 6, 7, 8, 9, 10 or more than 10 days. In some embodiments, the bacteriophage or bacteriophage cocktail is administered intravenously for 1 day and administered orally for at least 1, 2, 3, 4, 5, 6, 7, 8, 9 or 10 days. In some embodiments, the bacteriophage or bacteriophage cocktail is administered intravenously for 2 days and administered orally for at least 1, 2, 3, 4, 5, 6, 7, 8, 9 or 10 days. In some embodiments, the bacteriophage or bacteriophage cocktail is administered intravenously for 3 days and administered orally for at least 1, 2, 3, 4, 5, 6, 7, 8, 9 or 10 days. In some embodiments, the bacteriophage or bacteriophage cocktail is administered intravenously for 4 days and administered orally for at least 1, 2, 3, 4, 5, 6, 7, 8, 9 or 10 days. In some embodiments, the bacteriophage or bacteriophage cocktail is administered intravenously for 5 days and administered orally for at least 1, 2, 3, 4, 5, 6, 7, 8, 9 or 10 days.

In some embodiments, the bacteriophage or bacteriophage cocktail is administered intraurethrally for at least 1, 2, 3, 4, 5, 6, 7, 8, 9, 10 or more than 10 days. In some embodiments, the bacteriophage or bacteriophage cocktail is administered intraurethrally for 1 day and administered orally for at least 1, 2, 3, 4, 5, 6, 7, 8, 9 or 10 days. In some embodiments, the bacteriophage or bacteriophage cocktail is administered intraurethrally for 2 days and administered orally for at least 1, 2, 3, 4, 5, 6, 7, 8, 9 or 10 days. In some embodiments, the bacteriophage or bacteriophage cocktail is administered intraurethrally for 3 days and administered orally for at least 1, 2, 3, 4, 5, 6, 7, 8, 9 or 10 days. In some embodiments, the bacteriophage or bacteriophage cocktail is administered intraurethrally for 4 days and administered orally for at least 1, 2, 3, 4, 5, 6, 7, 8, 9 or 10 days. In some embodiments, the bacteriophage or bacteriophage cocktail is administered intraurethrally for 5 days and administered orally for at least 1, 2, 3, 4, 5, 6, 7, 8, 9 or 10 days.

In some embodiments, a dose of bacteriophage between 10³ and 10²⁰ PFU is given. In some embodiments, a dose of bacteriophage between 10³ and 10¹⁰ PFU is given. In some embodiments, a dose of bacteriophage between 10⁶ and 10²⁰ PFU is given. In some embodiments, a dose of bacteriophage between 10⁶ and 10¹⁰ PFU is given. For example, in some embodiments, the bacteriophage is present in a composition in an amount between 10³ and 10¹¹ PFU. In some embodiments, the bacteriophage is present in a composition in an amount about 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², 10²³, 10²⁴ PFU or more. In some embodiments, the bacteriophage is present in a composition in an amount of less than 10¹ PFU. In some embodiments, the bacteriophage is present in a composition in an amount between 10¹ and 10⁸, 10⁴ and 10⁹, 10⁵ and 10¹⁰, or 10⁷ and 10¹¹ PFU. In some embodiments, a composition comprising two or more bacteriophage is administered to a subject, wherein each bacteriophage is administered in an amount about 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹², 10¹⁸, 10¹⁹, 10²⁰, 10²¹, 10²², 10²³, 10²⁴ PFU or more. In some embodiments, a composition comprising two or more bacteriophage is administered to a subject, wherein each bacteriophage is administered in an amount of less than 10¹ PFU. In some embodiments, a composition comprising two or more bacteriophage is administered to a subject, wherein each bacteriophage is administered in an amount between 10¹ and 10⁸, 10⁴ and 10⁹, 10⁵ and 10¹⁰, or 10⁷ and 10¹¹ PFU. In some embodiments, a composition comprising two or more bacteriophage is administered to the subject, wherein each of the one or more bacteriophage is present in the composition at a concentration of at least about 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, or 10²⁰ PFU/mL. In some embodiments, the composition comprises less than about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², 10²³, 10²⁴ PFU/mL. In some embodiments, the composition comprises from about 1×10⁹ PFU to about 1×10¹² PFU/mL.

In some embodiments, a bacteriophage or a mixture is administered to a subject in need thereof 1, 2, 3, 4, 5, 6, 7, 8, 9, 10, 11, 12, 13, 14, 15, 16, 17, 18, 19, 20, 21, 22, 23, or 24 times a day. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, or 21 times a week. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month. In some embodiments, a bacteriophage or a mixture is administered to a subject in need thereof every 2, 4, 6, 8, 10, 12, 14, 18, 20, 22, or 24 hours.

In some embodiments, the compositions (bacteriophage) disclosed herein are administered before, during, or after the occurrence of a disease or condition. In some embodiment, the timing of administering the composition containing the bacteriophage varies. In some embodiments, the pharmaceutical compositions are used as a prophylactic and are administered continuously to subjects with a propensity to conditions or diseases in order to prevent the occurrence of the disease or condition. In some embodiments, pharmaceutical compositions are administered to a subject during or as soon as possible after the onset of the symptoms. In some embodiments, the administration of the compositions is initiated within the first 48 hours of the onset of the symptoms, within the first 24 hours of the onset of the symptoms, within the first 6 hours of the onset of the symptoms, or within 3 hours of the onset of the symptoms. In some embodiments, the initial administration of the composition is via any route practical, such as by any route described herein using any formulation described herein. In some embodiments, the compositions is administered as soon as is practicable after the onset of a disease or condition is detected or suspected, and for a length of time necessary for the treatment of the disease, such as, for example, from about 1 month to about 3 months. In some embodiments, the length of treatment will vary for each subject.

In some embodiments, after the initial treatment, the frequency of reoccurrence of the disease or condition is reduced compared to that of a subject not administered the treatment. In some embodiments, the disease or condition does not reoccur for at least one, two, three, four, five, six, seven, eight, nine, ten, eleven, twelve, or more than twelve months. In some embodiments, the disease or condition is a urinary tract infection.

In some embodiments, the bacteriophage or bacteriophage cocktail is administered concurrently with an antibiotic.

### Bacterial Infections

Disclosed herein, in certain embodiments, are methods of treating bacterial infections. In some embodiments, the bacteriophages disclosed herein treat or prevent diseases or conditions mediated or caused by bacteria as disclosed herein in a human or animal subject. In some embodiments, the bacteriophages disclosed herein treat or prevent diseases or conditions caused or exacerbated by bacteria as disclosed herein in a human or animal subject. Such bacteria are typically in contact with tissue of the subject including: gut, oral cavity, lung, armpit, ocular, vaginal, anal, ear, nose or throat tissue. In some embodiments, a bacterial infection is treated by modulating the activity of the bacteria and/or by directly killing of the bacteria.

In some embodiments, the bacterium is Escherichia spp. In some embodiments, the bacterium is Escherichia coli

In some embodiments, one or more Escherichia species present in a bacterial population are pathogenic. In some embodiments, the pathogenic bacteria are uropathogenic. In some embodiments, the pathogenic bacterium is uropathogenic E. coli (UPEC). In some embodiments, the pathogenic bacteria are diarrheagenic. In some embodiments, the pathogenic bacteria are diarrheagenic E. coli (DEC). In some embodiments, the pathogenic bacteria are Shiga-toxin producing. In some embodiments, the pathogenic bacterium is Shiga-toxin producing E. coli (STEC). In some embodiments, the pathogenic bacteria are Shiga-toxin producing. In some embodiments, the pathogenic bacterium is Shiga-toxin producing E. coli (STEC). In some embodiments, the pathogenic bacterium is Shiga-toxin producing E. coli (STEC). In some embodiments, the pathogenic bacteria are various O-antigen:H-antigen serotype E. coli. In some embodiments, the pathogenic bacteria are enteropathogenic. In some embodiments, the pathogenic bacterium is enteropathogenic E. coli (EPEC).

In some embodiments, the bacteriophages disclosed herein are used to treat an infection, a disease, or a condition, in the gastrointestinal tract of a subject. In some embodiments, the bacteriophages are used to modulate and/or kill target bacteria within the microbiome or gut flora of a subject. In some embodiments, the bacteriophages are used to selectively modulate and/or kill one or more target bacteria from a plurality of bacteria within the microbiome or gut flora of a subject. In some embodiments, the bacteriophages are used to selectively modulate and/or kill one or more target enteropathogenic bacteria from a plurality of bacteria within the microbiome or gut flora of a subject. In some embodiments, the target enteropathogenic bacterium is enteropathogenic E. coli (EPEC). In some embodiments, the bacteriophages are used to selectively modulate and/or kill one or more target diarrheagenic bacteria from a plurality of bacteria within the microbiome or gut flora of a subject. In some embodiments, the target diarrheagenic bacterium is diarrheagenic E. coli (DEC). In some embodiments, the bacteriophages are used to selectively modulate and/or kill one or more target Shiga-toxin producing bacteria from a plurality of bacteria within the microbiome or gut flora of a subject. In some embodiments, the target Shiga-toxin producing bacterium is Shiga-toxin producing E. coli (STEC).

In some embodiments, the bacteriophages disclosed herein are used to treat an infection, a disease, or a condition, in the urinary tract of a subject. In some embodiments, the bacteriophages are used to modulate and/or kill target bacteria within the urinary tract flora of a subject. The urinary tract flora includes, but is not limited, to Staphylococcus epidermidis, Enterococcus faecalis, and some alpha-hemolytic Streptococci. In some embodiments, the bacteriophages are used to selectively modulate and/or kill one or more target uropathogenic bacteria from a plurality of bacteria within the urinary tract flora of a subject. In some embodiments, the target bacterium is uropathogenic E. coli (UPEC). In some embodiments, the bacterial infection is a recurrent urinary tract infection.

In some embodiments, the bacteriophages disclosed herein are used to treat an infection, a disease, or a condition, on the skin of a subject. In some embodiments, the bacteriophages are used to modulate and/or kill target bacteria on the skin of a subject.

In some embodiments, the bacteriophages disclosed herein are used to treat an infection, a disease, or a condition, on a mucosal membrane of a subject. In some embodiments, the bacteriophages are used to modulate and/or kill target bacteria on the mucosal membrane of a subject.

In some embodiments, the pathogenic bacteria are antibiotic resistant. In some embodiments, the pathogenic bacteria are extended-spectrum beta-lactamase (ESBL) producing. In one embodiment, the pathogenic bacterium is carbapenem-resistant E. coli.

In some embodiments, the one or more target bacteria present in the bacterial population form a biofilm. In some embodiments, the biofilm comprises pathogenic bacteria. In some embodiments, the bacteriophage disclosed herein is used to treat a biofilm.

In some embodiments, the bacterium is includes Escherichia spp. In some embodiments, the bacterium is Escherichia coli.

In some embodiments, the bacteriophage treats acne and other related skin infections.

In some embodiments, the Escherichia species is a multiple drug resistant (MDR) bacteria strain. An MDR strain is a bacteria strain that is resistant to at least one antibiotic. In some embodiments, a bacteria strain is resistant to an antibiotic class such as a cephalosporin, a fluoroquinolone, a carbapenem, a colistin, an aminoglycoside, vancomycin, streptomycin, and methicillin. In some embodiments, the bacteria strain is Escherichia coli.

In some embodiments, the bacterium is E. coli. In some embodiments, the methods and compositions disclosed herein are for use in veterinary and medical applications as well as research applications.

### Microbiome

“Microbiome”, “microbiota”, and “microbial habitat” are used interchangeably hereinafter and refer to the ecological community of microorganisms that live on or in a subject's bodily surfaces, cavities, and fluids. Non-limiting examples of habitats of microbiome include: gut, colon, skin, skin surfaces, skin pores, vaginal cavity, umbilical regions, conjunctival regions, intestinal regions, stomach, nasal cavities and passages, gastrointestinal tract, urogenital tracts, saliva, mucus, and feces. In some embodiments, the microbiome comprises microbial material including, but not limited to, bacteria, archaea, protists, fungi, and viruses. In some embodiments, the microbial material comprises a gram-negative bacterium. In some embodiments, the microbial material comprises a gram-positive bacterium. In some embodiments, the microbial material comprises Proteobacteria, Actinobacteria, Bacteroidetes, or Firmicutes.

In some embodiments, the bacteriophages as disclosed herein are used to modulate or kill target bacteria within the microbiome of a subject. In some embodiments, the bacteriophages are used to modulate and/or kill target bacteria within the microbiome by the CRISPR-Cas system, lytic activity, or a combination thereof. In some embodiments, the bacteriophages are used to modulate and/or kill target bacteria within the microbiome of a subject. In some embodiments, the bacteriophages are used to selectively modulate and/or kill one or more target bacteria from a plurality of bacteria within the microbiome of a subject. In some embodiments, the target bacterium is E. coli. In some embodiments, the E. coli is a multidrug-resistant (MDR) strain. In some embodiments, the E. coli is an extended spectrum beta-lactamase (ESBL) strain. In some embodiments, the E. coli is a carbapenem-resistant strain. In some embodiments, the E. coli is a non-multidrug-resistant (non-MDR) strain. In some embodiments, the E. coli is a non-carbapenem-resistant strain. In some embodiments, the pathogenic bacteria are uropathogenic. In some embodiments, the pathogenic bacterium is uropathogenic E. coli (UPEC). In some embodiments, the pathogenic bacteria are diarrheagenic. In some embodiments, the pathogenic bacteria are diarrheagenic E. coli (DEC). In some embodiments, the pathogenic bacteria are Shiga-toxin producing. In some embodiments, the pathogenic bacterium is Shiga-toxin producing E. coli (STEC). In some embodiments, the pathogenic bacteria are various O-antigen:H-antigen serotype E. coli. In some embodiments, the pathogenic bacteria are enteropathogenic. In some embodiments, the pathogenic bacterium is enteropathogenic E. coli (EPEC).

In some embodiments, the bacteriophages are used to modulate or kill target single or plurality of bacteria within the microbiome or gut flora of the gastrointestinal tract of a subject. Modification (e.g., dysbiosis) of the microbiome or gut flora increases the risk for health conditions such as diabetes, mental disorders, ulcerative colitis, colorectal cancer, autoimmune disorders, obesity, diabetes, diseases of the central nervous system and inflammatory bowel disease. An exemplary bacteria associated with diseases and conditions of gastrointestinal tract and are being modulated or killed by the bacteriophages include strains, sub-strains, and enterotypes of E. coli.

In some embodiments, the bacteriophages are used to modulate or kill target single or plurality of bacteria within the microbiome or gut flora of the gastrointestinal tract of a subject. Modification (e.g., dysbiosis) of the microbiome or gut flora increases the risk for health conditions such as diabetes, mental disorders, ulcerative colitis, colorectal cancer, autoimmune disorders, obesity, diabetes, diseases of the central nervous system and inflammatory bowel disease. An exemplary list of the bacteria associated with diseases and conditions of gastrointestinal tract and are being modulated or killed by the bacteriophages include strains, sub-strains, and enterotypes of enterobacteriaceae, pasteurellaceae, fusobacteriaceae, neisseriaceae, veillonellaceae, gemellaceae, bacteriodales, clostridiales, erysipelotrichaceae, bifidobacteriaceae bacteroides, faecalibacterium, roseburia, blautia, ruminococcus, coprococcus, streptococcus, dorea, blautia, ruminococcus, lactobacillus, enterococcus, streptococcus, Escherichia coli, Fusobacterium nucleatum, Haemophilus parainfluenzae (pasteurellaceae), Veillonella parvula, Eikenella corrodens (neisseriaceae), Gemella moribillum, Bacteroides vulgatus, Bacteroides caccae, Bifidobacterium bifidum, Bifidobacterium longum, Bifidobacterium adolescentis, Bifidobacterium dentum, Blautia hansenii, Ruminococcus gnavus, Clostridium nexile, Faecalibacterium prausnitzii, Ruminoccus torques, Clostridium bolteae, Eubacterium rectale, Roseburia intestinalis, Coprococcus comes, actinomyces, lactococcus, roseburia, streptococcus, blautia, dialister, desulfovibrio, escherichia, lactobacillus, coprococcus, clostridium, bifidobacterium, klebsiella, granulicatella, eubacterium, anaerostipes, parabacteroides, coprobacillus, gordonibacter, collinsella, bacteroides, faecalibacterium, anaerotruncus, alistipes, haemophilus, anaerococcus, veillonella, arevotella, akkermansia, bilophila, sutterella, eggerthella, holdemania, gemella, peptoniphilus, rothia, enterococcus, pediococcus, citrobacter, odoribacter, enterobacteria, fusobacterium, and proteus.

In some embodiments, a bacteriophage disclosed herein is administered to a subject to promote a healthy microbiome. In some embodiments, a bacteriophage disclosed herein is administered to a subject to restore a subject's microbiome to a microbiome composition that promotes health. In some embodiments, a composition comprising a bacteriophage disclosed herein comprises a prebiotic or a third agent. In some embodiment, microbiome related disease or disorder is treated by a bacteriophage disclosed herein.

### Environmental Therapy

In some embodiments, bacteriophages disclosed herein are further used for food and agriculture sanitation (including meats, fruits and vegetable sanitation), hospital sanitation, home sanitation, vehicle and equipment sanitation, industrial sanitation, etc. In some embodiments, bacteriophages disclosed herein are used for the removal of antibiotic-resistant or other undesirable pathogens from medical, veterinary, animal husbandry, or any additional environments bacteria are passed to humans or animals.

Environmental applications of bacteriophage in health care institutions are for equipment such as endoscopes and environments such as ICUs which are potential sources of nosocomial infection due to pathogens that are difficult or impossible to disinfect. In some embodiments, a bacteriophage disclosed herein is used to treat equipment or environments inhabited by bacterial genera which become resistant to commonly used disinfectants. In some embodiments, bacteriophage compositions disclosed herein are used to disinfect inanimate objects. In some embodiments, an environment disclosed herein is sprayed, painted, or poured onto with aqueous solutions with bacteriophage titers. In some embodiment a solution described herein comprises between 101-1020 plaque forming units (PFU)/ml. In some embodiments, a bacteriophage disclosed herein is applied by aerosolizing agents that include dry dispersants to facilitate distribution of the bacteriophage into the environment. In some embodiments, objects are immersed in a solution containing bacteriophage disclosed herein.

### Sanitation

In some embodiments, bacteriophages disclosed herein are used as sanitation agents in a variety of fields. Although the terms “phage” or “bacteriophage” may be used, it should be noted that, where appropriate, this term should be broadly construed to include a single bacteriophage, multiple bacteriophages, such as a bacteriophage mixtures and mixtures of a bacteriophage with an agent, such as a disinfectant, a detergent, a surfactant, water, etc.

In some embodiments, bacteriophages are used to sanitize hospital facilities, including operating rooms, patient rooms, waiting rooms, lab rooms, or other miscellaneous hospital equipment. In some embodiments, this equipment includes electrocardiographs, respirators, cardiovascular assist devices, intraaortic balloon pumps, infusion devices, other patient care devices, televisions, monitors, remote controls, telephones, beds, etc. In some situations, the bacteriophage is applied through an aerosol canister. In some embodiments, bacteriophage is applied by wiping the bacteriophage on the object with a transfer vehicle.

In some embodiments, a bacteriophage described herein is used in conjunction with patient care devices. In some embodiment, bacteriophage is used in conjunction with a conventional ventilator or respiratory therapy device to clean the internal and external surfaces between patients. Examples of ventilators include devices to support ventilation during surgery, devices to support ventilation of incapacitated patients, and similar equipment. In some embodiments, the conventional therapy includes automatic or motorized devices, or manual bag-type devices such as are commonly found in emergency rooms and ambulances. In some embodiments, respiratory therapy includes inhalers to introduce medications such as bronchodilators as commonly used with chronic obstructive pulmonary disease or asthma, or devices to maintain airway patency such as continuous positive airway pressure devices.

In some embodiment, a bacteriophage described herein is used to cleanse surfaces and treat colonized people in an area where highly-contagious bacterial diseases, such as meningitis or enteric infections are present.

In some embodiments, water supplies are treated with a composition disclosed herein. In some embodiments, bacteriophage disclosed herein is used to treat contaminated water, water found in cisterns, wells, reservoirs, holding tanks, aqueducts, conduits, and similar water distribution devices. In some embodiments, the bacteriophage is applied to industrial holding tanks where water, oil, cooling fluids, and other liquids accumulate in collection pools. In some embodiments, a bacteriophage disclosed herein is periodically introduced to the industrial holding tanks in order to reduce bacterial growth.

In some embodiments, bacteriophages disclosed herein are used to sanitize a living area, such as a house, apartment, condominium, dormitory, or any living area. In some embodiments, the bacteriophage is used to sanitize public areas, such as theaters, concert halls, museums, train stations, airports, pet areas, such as pet beds, or litter boxes. In this capacity, the bacteriophage is dispensed from conventional devices, including pump sprayers, aerosol containers, squirt bottles, pre-moistened towelettes, etc, applied directly to (e.g., sprayed onto) the area to be sanitized, or be transferred to the area via a transfer vehicle, such as a towel, sponge, etc. In some embodiments, a bacteriophage disclosed herein is applied to various rooms of a house, including the kitchen, bedrooms, bathrooms, garage, basement, etc. In some embodiments, a bacteriophage disclosed herein is in the same manner as conventional cleaners. In some embodiments, the bacteriophage is applied in conjunction with (before, after, or simultaneously with) conventional cleaners provided that the conventional cleaner is formulated so as to preserve adequate bacteriophage biologic activity.

In some embodiments, a bacteriophage disclosed herein is added to a component of paper products, either during processing or after completion of processing of the paper products. Paper products to which a bacteriophage disclosed herein is added include, but are not limited to, paper towels, toilet paper, moist paper wipes.

### Food Safety

In some embodiments, a bacteriophage described herein is used in any food product or nutritional supplement, for preventing contamination. Examples for food or pharmaceuticals products are milk, yoghurt, curd, cheese, fermented milks, milk based fermented products, ice-creams, fermented cereal based products, milk based powders, infant formulae or tablets, liquid suspensions, dried oral supplement, wet oral supplement, or dry-tube-feeding.

The broad concept of bacteriophage sanitation is applicable to other agricultural applications and organisms. Produce, including fruits and vegetables, dairy products, and other agricultural products. For example, freshly-cut produce frequently arrive at the processing plant contaminated with pathogenic bacteria. This has led to outbreaks of food-borne illness traceable to produce. In some embodiments, the application of bacteriophage preparations to agricultural produce substantially reduce or eliminate the possibility of food-borne illness through application of a single bacteriophage or bacteriophage mixture with specificity toward species of bacteria associated with food-borne illness. In some embodiments, bacteriophages are applied at various stages of production and processing to reduce bacterial contamination at that point or to protect against contamination at subsequent points.

In some embodiments, specific bacteriophages are applied to produce in restaurants, grocery stores, produce distribution centers. In some embodiments, bacteriophages disclosed herein are periodically or continuously applied to the fruit and vegetable contents of a salad bar. In some embodiments, the application of bacteriophages to a salad bar or to sanitize the exterior of a food item is a misting or spraying process or a washing process.

In some embodiments, a bacteriophage described herein is used in matrices or support media containing with packaging containing meat, produce, cut fruits and vegetables, and other foodstuffs. In some embodiments, polymers that are suitable for packaging are impregnated with a bacteriophage preparation.

In some embodiments, a bacteriophage described herein is used in farm houses and livestock feed. In some embodiments, on a farm raising livestock, the livestock is provided with bacteriophage in their drinking water, food, or both. In some embodiments, a bacteriophage described herein is sprayed onto the carcasses and used to disinfect the slaughter area.

The use of specific bacteriophages as biocontrol agents on produce provides many advantages. For example, bacteriophages are natural, non-toxic products that will not disturb the ecological balance of the natural microflora in the way the common chemical sanitizers do, but will specifically lyse the targeted food-borne pathogens. Because bacteriophages, unlike chemical sanitizers, are natural products that evolve along with their host bacteria, new bacteriophages that are active against recently emerged, resistant bacteria are rapidly identified when required, whereas identification of a new effective sanitizer is a much longer process, several years.

### Pharmaceutical Compositions

Disclosed herein, in certain embodiments, are pharmaceutical compositions comprising (a) the nucleic acid sequences as disclosed herein; and (b) a pharmaceutically acceptable excipient. Also disclosed herein, in certain embodiments, are pharmaceutical compositions comprising (a) the bacteriophages as disclosed herein; and (b) a pharmaceutically acceptable excipient. Further disclosed herein, in certain embodiments, are pharmaceutical compositions comprising (a) the compositions as disclosed herein; and (b) a pharmaceutically acceptable excipient.

In some embodiments, the disclosure provides pharmaceutical compositions and methods of administering the same to treat bacterial, archaeal infections or to disinfect an area. In some embodiments, the pharmaceutical composition comprises any of the reagents discussed above in a pharmaceutically acceptable carrier. In some embodiments, a pharmaceutical composition or method disclosed herein treats urinary tract infections (UTI) and/or inflammatory diseases (e.g. inflammatory bowel disease (IBD)). In some embodiments, a pharmaceutical composition or method disclosed herein treats Crohn's disease. In some embodiments, a pharmaceutical composition or method disclosed herein treats ulcerative colitis.

In some embodiments, compositions disclosed herein comprise medicinal agents, pharmaceutical agents, carriers, adjuvants, dispersing agents, diluents, and the like.

In some embodiments, the bacteriophages disclosed herein are formulated for administration in a pharmaceutical carrier in accordance with suitable methods. In some embodiments, the manufacture of a pharmaceutical composition according to the disclosure, the bacteriophage is admixed with, inter alia, an acceptable carrier. In some embodiments, the carrier is a solid (including a powder) or a liquid, or both, and is preferably formulated as a unit-dose composition. In some embodiments, one or more bacteriophages are incorporated in the compositions disclosed herein, which are prepared by any suitable method of a pharmacy.

In some embodiments, the pharmaceutical compositions comprise a certain level of endotoxin. In some embodiments, the pharmaceutical compositions comprise less than about 100 EU/mL, 90 EU/mL, 80 EU/mL, 70 EU/mL, 60 EU/mL, 50 EU/mL, 40 EU/mL, 30 EU/mL, 20 EU/mL, 10 EU/mL, 9 EU/mL, 8 EU/mL, 7 EU/mL, 6 EU/mL, 5 EU/mL, 4 EU/mL, 2 EU/mL or 1 EU/mL endotoxin. In some embodiments, the pharmaceutical compositions comprise less than about 50 EU/mL endotoxin. In some embodiments, the pharmaceutical compositions comprise less than about 10 EU/mL endotoxin. In some embodiments, the pharmaceutical compositions comprises less than about 450 EU/dose, 400 EU/dose, 350 EU/dose, 300 EU/dose, 250 EU/dose, 200 EU/dose, 150 EU/dose, 100 EU/dose, 50 EU/dose, or 25 EU/dose.

In some embodiment, a method of treating subject's in-vivo, comprising administering to a subject a pharmaceutical composition comprising a bacteriophage disclosed herein in a pharmaceutically acceptable carrier, wherein the pharmaceutical composition is administered in a therapeutically effective amount. In some embodiments, the administration of the bacteriophage to a human subject or an animal in need thereof are by any means known in the art.

In some embodiments, bacteriophages disclosed herein are for oral administration. In some embodiments, the bacteriophages are administered in solid dosage forms, such as capsules, tablets, and powders, or in liquid dosage forms, such as elixirs, syrups, and suspensions. In some embodiments, compositions and methods suitable for buccal (sub-lingual) administration include lozenges comprising the bacteriophages in a flavored base, usually sucrose and acacia or tragacanth; and pastilles comprising the bacteriophages in an inert base such as gelatin and glycerin or sucrose and acacia.

In some embodiments, methods and compositions of the present disclosure are suitable for parenteral administration comprising sterile aqueous and non-aqueous injection solutions of the bacteriophage. In some embodiments, these preparations are isotonic with the blood of the intended recipient. In some embodiments, these preparations comprise antioxidants, buffers, bacteriostals and solutes which render the composition isotonic with the blood of the intended recipient. In some embodiments, aqueous and non-aqueous sterile suspensions include suspending agents and thickening agents. In some embodiments, compositions disclosed herein are presented in unit dose or multi-dose containers, for example sealed ampoules and vials, and are stored in a freeze-dried (lyophilized) condition requiring only the addition of the sterile liquid carrier, for example, saline or water for injection on immediately prior to use.

In some embodiment, methods and compositions suitable for rectal administration are presented as unit dose suppositories. In some embodiments, these are prepared by admixing the bacteriophage with one or more conventional solid carriers, for example, cocoa butter, and then shaping the resulting mixture. In some embodiments, methods and compositions suitable for topical application to the skin are in the form of an ointment, cream, lotion, paste, gel, spray, aerosol, or oil. In some embodiments, carriers which are used include petroleum jelly, lanoline, polyethylene glycols, alcohols, transdermal enhancers, and combinations of two or more thereof.

In some embodiments, methods and compositions suitable for transdermal administration are presented as discrete patches adapted to remain in intimate contact with the epidermis of the recipient for a prolonged period of time.

In some embodiments, methods and compositions suitable for nasal administration or otherwise administered to the lungs of a subject include any suitable means, e.g., administered by an aerosol suspension of respirable particles comprising the bacteriophage compositions, which the subject inhales. In some embodiments, the respirable particles are liquid or solid. As used herein, “aerosol” includes any gas-borne suspended phase, which is capable of being inhaled into the bronchioles or nasal passages. In some embodiments, aerosols of liquid particles are produced by any suitable means, such as with a pressure-driven aerosol nebulizer or an ultrasonic nebulizer. In some embodiments, aerosols of solid particles comprising the composition is produced with any solid particulate medicament aerosol generator, by techniques known in the pharmaceutical art.

In some embodiment, methods and compositions suitable for administering bacteriophages disclosed herein to a surface of an object or subject includes aqueous solutions. In some embodiments, such aqueous solutions are sprayed onto the surface of an object or subject. In some embodiment, the aqueous solutions are used to irrigate and clean a physical wound of a subject form foreign debris including bacteria.

In some embodiments, the bacteriophages disclosed herein are administered to the subject in a therapeutically effective amount. In some embodiments, at least one bacteriophage composition disclosed herein is formulated as a pharmaceutical formulation. In some embodiments, a pharmaceutical formulation comprises 1, 2, 3, 4, 5, 6, 7, 8, 9, 10, 11, 12, 13, 14, 15, 16, 17, 18, 19, 20 or more bacteriophage disclosed herein. In some instances, a pharmaceutical formulation comprises a bacteriophage described herein and at least one of: an excipient, a diluent, or a carrier.

In some embodiments, a pharmaceutical formulation comprises an excipient. Excipients are described in the Handbook of Pharmaceutical Excipients, American Pharmaceutical Association (1986) and includes but are not limited to solvents, dispersion media, diluents, or other liquid vehicles, dispersion or suspension aids, surface active agents, isotonic agents, thickening or emulsifying agents, preservatives, solid binders, and lubricants.

Non-limiting examples of suitable excipients include but is not limited to a buffering agent, a preservative, a stabilizer, a binder, a compaction agent, a lubricant, a chelator, a dispersion enhancer, a disintegration agent, a flavoring agent, a sweetener, a coloring agent.

In some embodiments, an excipient is a buffering agent. Non-limiting examples of suitable buffering agents include but is not limited to sodium citrate, magnesium carbonate, magnesium bicarbonate, calcium carbonate, and calcium bicarbonate. In some embodiments, a pharmaceutical formulation comprises any one or more buffering agent listed: sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts.

In some embodiments an excipient is a preservative. Non-limiting examples of suitable preservatives include but is not limited to antioxidants, such as alpha-tocopherol and ascorbate, and antimicrobials, such as parabens, chlorobutanol, and phenol. In some embodiments, antioxidants include but not limited to Ethylenediaminetetraacetic acid (EDTA), citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some embodiments, preservatives include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, protease inhibitor, reducing agent, alkylating agent, antimicrobial agent, oxidase inhibitor, or other inhibitor.

In some embodiments, a pharmaceutical formulation comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, the binders that are used in a pharmaceutical formulation are selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiments, a pharmaceutical formulation comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil. In some embodiments, lubricants that are in a pharmaceutical formulation are selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and tale or a combination thereof.

In some embodiments, an excipient comprises a flavoring agent. In some embodiments, flavoring agents includes natural oils; extracts from plants, leaves, flowers, and fruits; and combinations thereof.

In some embodiments, an excipient comprises a sweetener. Non-limiting examples of suitable sweeteners include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, xylitol, and the like.

In some instances, a pharmaceutical formulation comprises a coloring agent. Non-limiting examples of suitable color agents include food, drug and cosmetic colors (FD&C), drug and cosmetic colors (D&C), and external drug and cosmetic colors (Ext. D&C).

In some embodiments, the pharmaceutical formulation disclosed herein comprises a chelator. In some embodiments, a chelator includes ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA.

In some instances, a pharmaceutical formulation comprises a diluent. Non-limiting examples of diluents include water, glycerol, methanol, ethanol, and other similar biocompatible diluents. In some embodiments, a diluent is an aqueous acid such as acetic acid, citric acid, maleic acid, hydrochloric acid, phosphoric acid, nitric acid, sulfuric acid, or similar.

In some embodiments, a pharmaceutical formulation comprises a surfactant. In some embodiments, surfactants are be selected from, but not limited to, polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, amino acids such as L-leucine, sugar esters of fatty acids, glycerides of fatty acids or a combination thereof.

In some instances, a pharmaceutical formulation comprises an additional pharmaceutical agent. In some embodiments, an additional pharmaceutical agent is an antibiotic agent. In some embodiments, an antibiotic agent is of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides or tetracycline.

In some embodiments, an antibiotic agent described herein is an aminoglycoside such as Amikacin, Gentamicin, Kanamycin, Neomycin, Netilmicin, Tobramycin or Paromomycin. In some embodiments, an antibiotic agent described herein is an Ansamycin such as Geldanamycin or Herbimycin.

In some embodiments, an antibiotic agent described herein is a carbacephem such as Loracarbef. In some embodiments, an antibiotic agent described herein is a carbapenem such as Ertapenem, Doripenem, Imipenem/Cilastatin or Meropenem.

In some embodiments, an antibiotic agent described herein is a cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil or Cefuroxime. In some embodiments, an antibiotic agent is a Cephalosporins (third generation) such as Cefixime, Cefdinir, Cefditoren, Cefoperazone, Cefotaxime, Cefpodoxime, Ceftibuten, Ceftizoxime and Ceftriaxone or a Cephalosporins (fourth generation) such as Cefepime or Ceftobiprole.

In some embodiments, an antibiotic agent described herein is a lincosamide such as Clindamycin and Azithromycin, or a macrolide such as Azithromycin, Clarithromycin, Dirithromycin, Erythromycin, Roxithromycin, Troleandomycin, Telithromycin and Spectinomycin.

In some embodiments, an antibiotic agent described herein is a monobactams such as Aztreonam, or a nitrofuran such as Furazolidone or Nitrofurantoin.

In some embodiments, an antibiotic agent described herein is a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin.

In some embodiments, an antibiotic agent described herein is a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, or Trimethoprim-Sulfamethoxazole (Co-trimoxazole) (TMP-SMX).

In some embodiments, an antibiotic agent described herein is a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

In some embodiments, an antibiotic agent described herein is a polypeptide such as Bacitracin, Colistin or Polymyxin B.

In some embodiments, an antibiotic agent described herein is a tetracycline such as Demeclocycline, Doxycycline, Minocycline or Oxytetracycline.

### CERTAIN TERMINOLOGY

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

Unless the context indicates otherwise, it is specifically intended that the various features of the disclosure described herein are able of being used in any combination. Moreover, the present disclosure also contemplates that in some embodiments, any feature or combination of features set forth herein are excluded or omitted. To illustrate, if the specification states that a composition comprises components A, B and C, it is specifically intended that any of A, B or C, or a combination thereof, are omitted and disclaimed singularly or in any combination.

One of skill in the art will understand the interchangeability of terms designating the various CRISPR-Cas systems and their components due to a lack of consistency in the literature and an ongoing effort in the art to unify such terminology.

As used in the description and the appended claims, the singular forms “a,” “an” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

The term “about” as used herein when referring to a measurable value such as a dosage or time period and the like refers to variations of ±20%, ±10%, ±5%, ±1%, ±0.5%, or even ±0.1% of the specified amount. As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise”, “comprises”, and “comprising”, “includes”, “including”, “have” and “having”, as used herein, specify the presence of the stated features, steps, operations, elements, and/or components, but do not preclude the presence or addition of one or more other features, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising.”

The term “consists of” and “consisting of”, as used herein, excludes any features, steps, operations, elements, and/or components not otherwise directly stated. The use of “consisting of” limits only the features, steps, operations, elements, and/or components set forth in that clause and does exclude other features, steps, operations, elements, and/or components from the claim as a whole.

In some embodiments, as used herein “a part of” or “a portion of” or similar language includes at least 10 contiguous nucleobases or amino acids, as applicable.

The terms “complementary” or “complementarity”, as used herein, refer to the natural binding of polynucleotides under permissive salt and temperature conditions by base-pairing. For example, the sequence “A-G-T” binds to the complementary sequence “T-C-A.” Complementarity between two single-stranded molecules is “partial,” in which only some of the nucleotides bind, or it is complete when total complementarity exists between the single stranded molecules. The degree of complementarity between nucleic acid strands has significant effects on the efficiency and strength of hybridization between nucleic acid strands.

“Complement” as used herein means 100% complementarity or identity with the comparator nucleotide sequence or it means less than 100% complementarity (e.g., about 80%, 81%, 82%, 83%, 84%, 85%, 86%, 87%, 88%, 89%, 90%, 91%, 92%, 93%, 94%, 95%, 96%, 97%, 98%, 99%, and the like, complementarity). Complement or complementable may also be used in terms of a “complement” to or “complementing” a mutation.

As used herein, the term “CRISPR bacteriophage”, “CRISPR enhanced bacteriophage”, and “crPhage” refers to a bacteriophage particle comprising bacteriophage DNA comprising at least one heterologous polynucleotide that encodes at least one component of a CRISPR-Cas system (e.g., CRISPR array, crRNA; e.g., P1 bacteriophage comprising an insertion of a targeting crRNA). In some embodiments, the polynucleotide encodes at least one transcriptional activator of a CRISPR-Cas system. In some embodiments, the polynucleotide encodes at least one component of an anti-CRISPR polypeptide of a CRISPR-Cas system.

As used herein, the phrase “substantially identical,” or “substantial identity” in the context of two nucleic acid molecules, nucleotide sequences or protein sequences, refers to two or more sequences or subsequences that have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments, substantial identity refers to two or more sequences or subsequences that have at least about 70%, at least about 75%, at least about 80%, at least about 85%, at least about 90%, at least about 95, 96, 96, 97, 98, or 99% identity. For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences is to a full-length polynucleotide sequence or to a portion thereof, or to a longer polynucleotide sequence. In some instances, “Percent identity” is determined using BLASTX version 2.0 for translated nucleotide sequences and BLASTN version 2.0 for polynucleotide sequences.

As used herein, a “target nucleotide sequence” refers to the portion of a target gene (i.e., target region in the genome or the “protospacer sequence,” which is adjacent to a protospacer adjacent motif (PAM) sequence) that is fully complementary or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a CRISPR array.

As used herein, the term “protospacer adjacent motif” or “PAM” refers to a DNA sequence present on the target DNA molecule adjacent to the nucleotide sequence matching the spacer sequence. This motif is found in the target gene next to the region to which a spacer sequence binds as a result of being complementary to that region and identifies the point at which base pairing with the spacer nucleotide sequence begins. The exact PAM sequence that is required varies between each different CRISPR-Cas system. Non-limiting examples of PAMs include CCA, CCT, CCG, TTC, AAG, AGG, ATG, GAG, and/or CC. In some instances, in Type I systems, the PAM is located immediately 5′ to the sequence that matches the spacer, and thus is 3′ to the sequence that base pairs with the spacer nucleotide sequence, and is directly recognized by Cascade. In some instances, for B. halodurans Type I-C systems, the PAM is YYC, where Y can be either T or C. In some instances, for the P. aeruginosa Type I-C system, the PAM is TTC. Once a cognate protospacer and PAM are recognized, Cas3 is recruited, which then cleaves and degrades the target DNA. For Type II systems, the PAM is required for a Cas9/sgRNA to form an R-loop to interrogate a specific DNA sequence through Watson-Crick pairing of its guide RNA with the genome. The PAM specificity is a function of the DNA-binding specificity of the Cas9 protein (e.g., a -protospacer adjacent motif recognition domain at the C-terminus of Cas9).

As used herein, the term “gene” refers to a nucleic acid molecule capable of being used to produce mRNA, tRNA, rRNA, miRNA, anti-microRNA, regulatory RNA, and the like. Genes may or may not be capable of being used to produce a functional protein or gene product. Genes include both coding and non-coding regions (e.g., introns, regulatory elements, promoters, enhancers, termination sequences and/or 5′ and 3′ untranslated regions). A gene is “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

By the terms “treat,” “treating,” or “treatment,” it is intended that the severity of the subject's condition is reduced or at least partially improved or modified and that some alleviation, mitigation or decrease in at least one clinical symptom is achieved, and/or there is a delay in the progression of the disease or condition, and/or delay of the onset of a disease or illness. With respect to an infection, a disease or a condition, the term refers to a decrease in the symptoms or other manifestations of the infection, disease or condition. In some embodiments, treatment provides a reduction in symptoms or other manifestations of the infection, disease or condition by at least about 5%, e.g., about 10%, 15%, 20%, 25%, 30%, 35%, 40%, 45%, 50% or more.

The terms “prevent,” “preventing,” and “prevention” (and grammatical variations thereof) refer to prevention and/or delay of the onset of an infection, disease, condition and/or a clinical symptom(s) in a subject and/or a reduction in the severity of the onset of the infection, disease, condition and/or clinical symptom(s) relative to what would occur in the absence of carrying out the methods disclosed herein prior to the onset of the disease, disorder and/or clinical symptom(s). Thus, in some embodiments, to prevent infection, food, surfaces, medical tools and devices are treated with compositions and by methods disclosed herein.

The terms with respect to an “infection”, “a disease”, or “a condition”, used herein, refer to any adverse, negative, or harmful physiological condition in a subject. In some embodiments, the source of an “infection”, “a disease”, or “a condition”, is the presence of a target bacterial population in and/or on a subject. In some embodiments, the bacterial population comprises one or more target bacterial species. In some embodiments, the one or more bacteria species in the bacterial population comprise one or more strains of one or more bacteria. In some embodiments, the target bacterial population causes an “infection”, “a disease”, or “a condition” that is acute or chronic. In some embodiments, the target bacterial population causes an “infection”, “a disease”, or “a condition” that is localized or systemic. In some embodiments, the target bacterial population causes an “infection”, “a disease”, or “a condition” that is idiopathic. In some embodiments, the target bacterial population causes an “infection”, “a disease”, or “a condition” that is acquired through means, including but not limited to, respiratory inhalation, ingestion, skin and wound infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, infections of indwelling medical devices such as joint prostheses, dental implants, catheters and cardiac implants, sexual contact, and/or hospital-acquired and ventilator-associated bacterial pneumonias.

The terms “individual”, or “subject” as used herein includes any animal that has or is susceptible to an infection, disease or condition involving bacteria. Thus, in some embodiments, subjects are mammals, avians, reptiles, amphibians, fish, crustaceans, or mollusks. Mammalian subjects include but are not limited to humans, non-human primates (e.g., gorilla, monkey, baboon, and chimpanzee, etc.), dogs, cats, goats, horses, pigs, cattle, sheep, and the like, and laboratory animals (e.g., rats, guinea pigs, mice, gerbils, hamsters, and the like). Avian subjects include but are not limited to chickens, ducks, turkeys, geese, quail, pheasants, and birds kept as pets (e.g., parakeets, parrots, macaws, cockatoos, canaries, and the like). Fish subjects include but are not limited to species used in aquaculture (e.g., tuna, salmon, tilapia, catfish, carp, trout, cod, bass, perch, snapper, and the like). Crustacean subjects include but are not limited to species used in aquaculture (e.g., shrimp, prawn, lobster, crayfish, crab and the like). Mollusk subjects include but are not limited to species used in aquaculture (e.g., abalone, mussel, oyster, clams, scallop and the like). In some embodiments, suitable subjects include both males and females and subjects of any age, including embryonic (e.g., in-utero or in-ovo), infant, juvenile, adolescent, adult and geriatric subjects. In some embodiments, a subject is a human.

As used here the term “isolated” in context of a nucleic acid sequence is a nucleic acid sequence that exists apart from its native environment.

As used herein, “expression cassette” means a recombinant nucleic acid molecule comprising a nucleotide sequence of interest (e.g., the recombinant nucleic acid molecules and CRISPR arrays disclosed herein), wherein the nucleotide sequence is operably associated with at least a control sequence (e.g., a promoter).

As used herein, “chimeric” refers to a nucleic acid molecule or a polypeptide in which at least two components are derived from different sources (e.g., different organisms, different coding regions).

As used herein, “selectable marker” means a nucleotide sequence that when expressed imparts a distinct phenotype to the host cell expressing the marker and thus allows such transformed cells to be distinguished from those that do not have the marker.

As used herein, “vector” refers to a composition for transferring, delivering or introducing a nucleic acid (or nucleic acids) into a cell.

As used herein, “pharmaceutically acceptable” means a material that is not biologically or otherwise undesirable, i.e., the material are administered to a subject without causing any undesirable biological effects such as toxicity.

As used herein the term “biofilm” means an accumulation of microorganisms embedded in a matrix of polysaccharide. Biofilms form on solid biological or non-biological surfaces and are medically important, accounting for over 80 percent of microbial infections in the body.

As used herein, the term “in vivo” is used to describe an event that takes place in a subject's body.

As used herein, the term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, “homolog” of a first protein or first gene derived from a first organism (also referred to as a “homologous gene” or “homologous protein”) refers to a second protein or a second gene that is present in a second organism, wherein the first organism and the second organism share a common ancestor. A homolog may have at least 50%, 60%, 70%, 80%, 85%, 90%, 95%, 97.5%, 98%, 99%, or 100% sequence identity with the first gene or first protein.

### Further Embodiments

1. A bacteriophage comprising a CRISPR system comprising:
   (a) a CRISPR array comprising a spacer sequence complementary to a target nucleotide sequence in an Escherichia species; and
   (b) a nucleic acid sequence encoding a CRISPR associated nuclease.
2. The bacteriophage of embodiment 1, wherein the spacer sequence comprises at least about 90%, 91%, 92%, 93%, 94%, 95%, 96%, 97%, 98%, or 99% sequence identity to any one of SEQ ID NOS: 12 or 20-37.
3. The bacteriophage of embodiment 1 or embodiment 2, wherein the spacer sequence comprises a first spacer sequence and a second spacer sequence, wherein the first spacer sequence is complementary to the target nucleotide sequence in the Escherichia species, and the second spacer sequence is complementary to a second target nucleotide sequence in the Escherichia species and/or a second Escherichia species; optionally wherein the spacer sequence comprises a third spacer sequence, wherein the third spacer sequence is complementary to a third target nucleotide sequence in the Escherichia species, second Escherichia species, and/or third Escherichia species.
4. The bacteriophage of any one of embodiments 1-3, wherein the CRISPR array further comprises at least one repeat sequence, wherein optionally the at least one repeat sequence is operably linked to the spacer sequence at either its 5′ end or its 3′ end.
5. The bacteriophage of embodiment 4, wherein the repeat sequence comprises at least about 90%, 91%, 92%, 93%, 94%, 95%, 96%, 97%, 98%, or 99% sequence identity to any one of SEQ ID NOs: 13-18.
6. The bacteriophage of any one of embodiments 1-5, wherein the CRISPR array comprises at least about 90%, 91%, 92%, 93%, 94%, 95%, 96%, 97%, 98%, or 99% sequence identity to a sequence selected from SEQ ID NOS: 38-45.
7. The bacteriophage of any one of embodiments 1-6, wherein the target nucleotide sequence comprises a coding sequence.
8. The bacteriophage of any one of embodiments 1-6, wherein the target nucleotide sequence comprises a non-coding or intergenic sequence.
9. The bacteriophage of any one of embodiments 1-6, wherein the target nucleotide sequence comprises all or a part of (e.g., at least 10 contiguous nucleobases) a promoter sequence.
10. The bacteriophage of embodiment 9, wherein the promoter sequence comprises at least about 90%, 91%, 92%, 93%, 94%, 95%, 96%, 97%, 98%, or 99% sequence identity to any one of SEQ ID NOs: 1-11 or 19.
11. The bacteriophage of any one of embodiments 1-10, wherein the target nucleotide sequence comprises all or a part of (e.g., at least 10 contiguous nucleobases) a nucleotide sequence located on a coding strand of a transcribed region of an essential gene, or a non-essential gene.
12. The bacteriophage of embodiment 11, wherein the target nucleotide sequence comprises all of a part of (e.g., at least 10 contiguous nucleobases) a nucleotide sequence located on a coding strand of a transcribed region of an essential gene, and the essential gene is Tsf acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK.
13. The bacteriophage of any one of embodiments 1-12, wherein the CRISPR system comprises a CRISPR-Cpf1 system, a Type II CRISPR-Cas system, or a Type V CRISPR-Cas system.
14. The bacteriophage of any one of embodiments 1-12, wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
15. The bacteriophage of embodiment 14, wherein the Cascade complex comprises: (i) a Cas5d polypeptide, a Cas8c polypeptide, and a Cas7 polypeptide (Type I-C CRISPR-Cas system); (ii) a Cas7 polypeptide, a Cas8a1 polypeptide or a Cas8a2 polypeptide, a Cas5 polypeptide, a Csa5 polypeptide, and a Cas6a polypeptide, a Cas3′ polypeptide, and a Cas3″ polypeptide having no nuclease activity (Type I-A CRISPR-Cas system); (iii) a Cas6b polypeptide, a Cas8b polypeptide, a Cas7 polypeptide, and a Cas5 polypeptide (Type I-B CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
16. The bacteriophage of embodiment 14, wherein the Cascade complex comprises a Cas5d polypeptide, a Cas8c polypeptide, and a Cas7 polypeptide (Type I-C CRISPR-Cas system).
17. The bacteriophage of any one of embodiments 14-16, wherein the Cascade complex comprises a Pseudomonas aeruginosa Type I-C Cascade complex.
18. The bacteriophage of any one of embodiments 1-17, further comprising a nucleic acid sequence comprising a promoter sequence.
19. The bacteriophage of any one of embodiments 1-18, wherein the bacteriophage infects multiple bacterial strains.
20. The bacteriophage of any one of embodiments 1-19, wherein the bacteriophage is an obligate lytic bacteriophage.
21. The bacteriophage of any one of embodiments 1-20, wherein the bacteriophage is a temperate bacteriophage that is rendered lytic.
22. The bacteriophage of embodiment 21, wherein the temperate bacteriophage is rendered lytic by the removal, replacement, or inactivation of a lysogeny gene.
23. The bacteriophage of any one of embodiments 20-22, wherein the Escherichia species is killed solely by lytic activity of the bacteriophage.
24. The bacteriophage of any one of embodiments 20-22, wherein the Escherichia species is killed solely by activity of the CRISPR system.
25. The bacteriophage of any one of embodiments 20-22, wherein the Escherichia species is killed by lytic activity of the bacteriophage in combination with activity of the CRISPR system.
26. The bacteriophage of embodiment 25, wherein the Escherichia species is killed by the activity of the CRISPR system, independently of the lytic activity of the bacteriophage.
27. The bacteriophage of embodiment 25, wherein the activity of the CRISPR system supplements or enhances the lytic activity of the bacteriophage.
28. The bacteriophage of embodiment 25, wherein the lytic activity of the bacteriophage and the activity of the CRISPR system are synergistic.
29. The bacteriophage of any one of embodiments 20-26, wherein the lytic activity of the bacteriophage, the activity of the CRISPR system, or both, is modulated by a concentration of the bacteriophage.
30. The bacteriophage of any one of embodiments 1-29, wherein the bacteriophage comprises a Tequatrovirus, a Mosigyvirus, a Phapecoctavirus, a Unique Myoviridae, or a Vectrevirus.
31. The bacteriophage of any one of embodiments 1-30, wherein the bacteriophage comprises at least
80% sequence identity to a bacteriophage selected from p004K, p00c0, p00ex, p00jc, p00ke, or p5516.
32. The bacteriophage of embodiment 31, wherein the bacteriophage is p004Ke007, p004Ke009, p00c0e030, p00c0e103, or p00exe014.
33. The bacteriophage of any one of embodiments 1-32, wherein the nucleic acid sequence is inserted into a non-essential bacteriophage gene.
34. A bacteriophage comprising a CRISPR system comprising:
   a. a CRISPR array comprising a first spacer sequence comprising a sequence selected from SEQ ID NOS: 12 or 20-37; and
   b. a nucleic acid sequence encoding a CRISPR associated nuclease.
35. The bacteriophage of embodiment 34, wherein the first spacer sequence comprises SEQ ID NO: 12, SEQ ID NO: 25, or SEQ ID NO: 24.
36. The bacteriophage of embodiment 34, wherein the CRISPR array comprises a second spacer sequence.
37. The bacteriophage of embodiment 36, wherein the first spacer sequence comprises SEQ ID NO: 12, and the second spacer sequence comprises SEQ ID NO: 25, or the first spacer sequence comprises SEQ ID NO: 12, and the second spacer sequence comprises SEQ ID NO: 24, or the first spacer sequence comprises SEQ ID NO: 25, and the second spacer sequence comprises SEQ ID NO: 24.
38. The bacteriophage of embodiment 37, wherein the CRISPR array comprises a third spacer sequence.
39. The bacteriophage of embodiment 38, wherein the first spacer sequence comprises SEQ ID NO: 12, the second spacer sequence comprises SEQ ID NO: 25, and the third spacer sequence comprises SEQ ID NO: 24.
40. A pharmaceutical composition comprising:
   (a) the bacteriophage of any one of embodiments 1-39; and
   (b) a pharmaceutically acceptable excipient.
41. The pharmaceutical composition of embodiment 40, wherein the pharmaceutical composition comprises at least two bacteriophage.
42. The pharmaceutical composition of embodiment 40 or embodiment 41, wherein the bacteriophage are from the lineage consisting of a Tequatrovirus, a Mosigyvirus, a Phapecoctavirus, a Unique Myoviridae, or a Vectrevirus.
43. The pharmaceutical composition of embodiment 41 or embodiment 42, wherein the pharmaceutical composition comprises at least six bacteriophage, wherein the bacteriophage comprise p004k, p00c0, p00ex, p00jc, p00ke, and p5516; or p00exe014, p004Ke009, p00jc, p00ke, p00c0e030, p00c0e103, and p5516.
44. The pharmaceutical composition of any one of embodiments 40-43, wherein the pharmaceutical composition is in the form of a tablet, a capsule, a liquid, a syrup, an oral formulation, an intravenous formulation, an intranasal formulation, an ocular formulation, an otic formulation, a subcutaneous formulation, a topical formulation, a transdermal formulation, a transmucosal formulation, an inhalable respiratory formulation, a suppository, a lyophilized formulation, a nebulizable formulation, and any combination thereof.
45. A method of killing an Escherichia species comprising introducing into the Escherichia species genetic material from a bacteriophage comprising a CRISPR system comprising:
   (a) a CRISPR array comprising a spacer sequence complementary to target nucleotide sequence in the Escherichia species; and
   (b) a nucleic acid sequence encoding a CRISPR associated nuclease.
46. The method of embodiment 45, wherein the spacer sequence comprises at least about 90%, 91%, 92%, 93%, 94%, 95%, 96%, 97%, 98%, or 99% sequence identity to any one of SEQ ID NOS: 12 or 20-37.
47. The method of embodiment 45 or embodiment 46, wherein the spacer sequence comprises a first spacer sequence and a second spacer sequence, wherein the first spacer sequence is complementary to the target nucleotide sequence in the Escherichia species, and the second spacer sequence is complementary to a second target nucleotide sequence in the Escherichia species and/or a second Escherichia species; optionally wherein the spacer sequence comprises a third spacer sequence, wherein the third spacer sequence is complementary to a third target nucleotide sequence in the Escherichia species, second Escherichia species, and/or third Escherichia species.
48. The method of any one of embodiments 45-47, wherein the CRISPR array further comprises at least one repeat sequence, wherein optionally the at least one repeat sequence is operably linked to the spacer sequence at either its 5′ end or its 3′ end.
49. The method of embodiment 48, wherein the repeat sequence comprises at least about 90%, 91%, 92%, 93%, 94%, 95%, 96%, 97%, 98%, or 99% sequence identity to any one of SEQ ID NOs: 13-18.
50. The method of any one of embodiments 45-49, wherein the CRISPR array comprises at least about 90%, 91%, 92%, 93%, 94%, 95%, 96%, 97%, 98%, or 99% sequence identity to a sequence selected from SEQ ID NOS: 38-45.
51. The method of any one of embodiments 45-50, wherein the target nucleotide sequence comprises a coding sequence.
52. The method of any one of embodiments 45-50, wherein the target nucleotide sequence comprises a non-coding or intergenic sequence.
53. The method of any one of embodiments 45-52, wherein the target nucleotide sequence comprises all or a part of (e.g., at least 10 contiguous nucleobases) a promoter sequence.
54. The method of embodiment 53, wherein the promoter sequence comprises at least about 90%, 91%, 92%, 93%, 94%, 95%, 96%, 97%, 98%, or 99% sequence identity to any one of SEQ ID NOs: 1-11 or 19.
55. The method of any one of embodiments 45-54, wherein the target nucleotide sequence comprises all or a part of (e.g., at least 10 contiguous nucleobases) a nucleotide sequence located on a coding strand of a transcribed region of an essential gene, or a non-essential gene.
56. The method of embodiment 55, wherein the target nucleotide sequence comprises all of a part of (e.g., at least 10 contiguous nucleobases) a nucleotide sequence located on a coding strand of a transcribed region of an essential gene, and the essential gene is Tsf acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK.
57. The method of any one of embodiments 45-56, wherein the CRISPR system comprises a CRISPR-Cpf1 system, a Type II CRISPR-Cas system, or a Type V CRISPR-Cas system.
58. The method of any one of embodiments 45-56, wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
59. The method of embodiment 58, wherein the Cascade complex comprises:
   (i) a Cas5d polypeptide, a Cas8c polypeptide, and a Cas7 polypeptide (Type I-C CRISPR-Cas system); (ii) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (iii) a Cas6b polypeptide, a Cas8b polypeptide, a Cas7 polypeptide, and a Cas5 polypeptide (Type I-B CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
60. The method of embodiment 58, wherein the Cascade complex comprises a Cas5d polypeptide, a Cas8c polypeptide, and a Cas7 polypeptide (Type I-C CRISPR-Cas system).
61. The method of embodiment 60, wherein the Cascade complex comprises a Pseudomonas aeruginosa Type I-C Cascade complex.
62. The method of any one of embodiments 45-61, wherein the CRISPR system further comprises a nucleic acid sequence comprising a promoter sequence.
63. The method of any one of embodiments 45-62, wherein the bacteriophage is an obligate lytic bacteriophage.
64. The method of any one of embodiments 45-63, wherein the bacteriophage is a temperate bacteriophage that is rendered lytic.
65. The method of embodiment 64, wherein the temperate bacteriophage is rendered lytic by the removal, replacement, or inactivation of a lysogeny gene.
66. The method of any one of embodiments 45-65, wherein the Escherichia species is killed solely by activity of the CRISPR system.
67. The method of any one of embodiments 57-66, wherein the Escherichia species is killed by lytic activity of the bacteriophage in combination with activity of the CRISPR system.
68. The method of embodiment 67, wherein the Escherichia species is killed by the activity of the CRISPR system, independently of the lytic activity of the bacteriophage.
69. The method of embodiment 67, wherein the activity of the CRISPR system supplements or enhances the lytic activity of the bacteriophage.
70. The method of embodiment 67, wherein the lytic activity of the bacteriophage and the activity of the CRISPR system are synergistic.
71. The method of any one of embodiments 66-70, wherein the lytic activity of the bacteriophage, the activity of the CRISPR system, or both, is modulated by a concentration of the bacteriophage.
72. The method of any one of embodiments 45-71, wherein the bacteriophage infects multiple bacterial strains.
73. The method of any one of embodiments 45-72, wherein the bacteriophage comprises at least 80% identity to p004K, p00c0, p00ex, p00jc, p00ke, p5516, p004Ke007, p004Ke009, p00c0e030, p00c0e103 or p00exe014.
74. The method of any one of embodiments 45-73, wherein the nucleic acid sequence is inserted in place of or adjacent to a non-essential bacteriophage gene.
75. The method of any one of embodiments 45-74, wherein a mixed population of bacterial cells comprises the Escherichia species.
76. A method of treating a disease in an individual in need thereof, the method comprising administering to the individual a bacteriophage comprising a CRISPR system comprising:
   (a) a CRISPR array comprising a spacer sequence complementary to target nucleotide sequence in an Escherichia species; and
   (b) a nucleic acid sequence encoding a CRISPR associated nuclease.
77. The method of embodiment 76, wherein the spacer sequence comprises at least about 90%, 91%, 92%, 93%, 94%, 95%, 96%, 97%, 98%, or 99% sequence identity to any one of SEQ ID NOS: 12 or 20-37.
78. The method of embodiment 76 or embodiment 77, wherein the spacer sequence comprises a first spacer sequence and a second spacer sequence, wherein the first spacer sequence is complementary to the target nucleotide sequence in the Escherichia species, and the second spacer sequence is complementary to a second target nucleotide sequence in the Escherichia species and/or a second Escherichia species; optionally wherein the spacer sequence comprises a third spacer sequence, wherein the third spacer sequence is complementary to a third target nucleotide sequence in the Escherichia species, second Escherichia species, and/or third Escherichia species.
79. The method of embodiment 76 or embodiment 77, wherein the CRISPR array further comprises at least one repeat sequence, wherein optionally the at least one repeat sequence is operably linked to the spacer sequence at either its 5′ end or its 3′ end.
80. The method of embodiment 79, wherein the repeat sequence comprises at least about 90%, 91%, 92%, 93%, 94%, 95%, 96%, 97%, 98%, or 99% sequence identity to any one of SEQ ID NOs: 13-18.
81. The method of any one of embodiments 76-80, wherein the CRISPR array comprises at least about 90%, 91%, 92%, 93%, 94%, 95%, 96%, 97%, 98%, or 99% sequence identity to a sequence selected from SEQ ID NOS: 38-45.
82. The method of any one of embodiments 76-81, wherein the target nucleotide sequence comprises a coding sequence.
83. The method of any one of embodiments 76-81, wherein the target nucleotide sequence comprises a non-coding or intergenic sequence.
84. The method of any one of embodiments 76-83, wherein the target nucleic acid sequence comprises all or a part of (e.g., at least 10 contiguous nucleobases) a promoter sequence.
85. The method of embodiment 84, wherein the promoter sequence comprises at least about 90%, 91%, 92%, 93%, 94%, 95%, 96%, 97%, 98%, or 99% sequence identity to any one of SEQ ID NOs: 1-11 or 19.
86. The method of any one of embodiments 76-85, wherein the target nucleotide sequence comprises all or a part of (e.g., at least 10 contiguous nucleobases) a nucleotide sequence located on a coding strand of a transcribed region of an essential gene, or a non-essential gene.
87. The method of embodiment 86, wherein the target nucleotide sequence comprises all of a part of (e.g., at least 10 contiguous nucleobases) a nucleotide sequence located on a coding strand of a transcribed region of an essential gene, and the essential gene is Tsf acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK.
88. The method of any one of embodiments 76-87, wherein the CRISPR system comprises a CRISPR-Cpf1 system, a Type II CRISPR-Cas system, or a Type V CRISPR-Cas system.
89. The method of any one of embodiments 76-87, wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
90. The method of embodiment 89, wherein the Cascade complex comprises:
   (i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (ii) a Cas6b polypeptide, a Cas8b polypeptide, a Cas7 polypeptide, and a Cas5 polypeptide (Type I-B CRISPR-Cas system); (iii) a Cas5d polypeptide, a Cas8c polypeptide, and a Cas7 polypeptide (Type I-C CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
91. The method of embodiment 90, wherein the Cascade complex comprises a Cas5d polypeptide, a Cas8c polypeptide, and a Cas7 polypeptide (Type I-C CRISPR-Cas system).
92. The method of any one of embodiments 76-91, wherein the CRISPR system further comprises a nucleic acid sequence comprising a promoter sequence.
93. The method of any one of embodiments 76-92, wherein the bacteriophage is an obligate lytic bacteriophage.
94. The method of any one of embodiments 76-92, wherein the bacteriophage is a temperate bacteriophage that is rendered lytic.
95. The method of embodiment 94, wherein the temperate bacteriophage is rendered lytic by the removal, replacement, or inactivation of a lysogeny gene.
96. The method of any one of embodiments 93-95, wherein the Escherichia species is killed solely by activity of the CRISPR system.
97. The method of any one of embodiments 93-95, wherein the Escherichia species is killed by lytic activity of the bacteriophage in combination with activity of the CRISPR system.
98. The method of embodiment 97, wherein the Escherichia species is killed by the activity of the CRISPR system, independently of the lytic activity of the bacteriophage.
99. The method of embodiment 97, wherein the activity of the CRISPR system supplements or enhances the lytic activity of the bacteriophage.
100. The method of embodiment 97, wherein the lytic activity of the bacteriophage and the activity of the CRISPR system are synergistic.
101. The method of any one of embodiments 93-100, wherein the lytic activity of the bacteriophage, the activity of the CRISPR system, or both, is modulated by a concentration of the bacteriophage.
102. The method of any one of embodiments 76-101, wherein the bacteriophage infects multiple bacterial strains.
103. The method of any one of embodiments 76-102, wherein the bacteriophage comprises at least 80% identity to p004K, p00c0, p00ex, p00jc, p00ke, p5516, p004Ke007, p004Ke009, p00c0e030, p00c0e103, or p00exe014.
104. The method of any one of embodiments 76-103, wherein the nucleic acid sequence is inserted in place of or adjacent to a non-essential bacteriophage gene.
105. The method of any one of embodiments 76-104, wherein the disease is a bacterial infection.
106. The method of any one of embodiments 76-105, wherein the Escherichia species causing the disease is a drug resistant Escherichia species.
107. The method of embodiment 106, wherein the drug resistant Escherichia species is resistant to at least one antibiotic.
108. The method of any one of embodiments 76-107, wherein the Escherichia species causing the disease is a multidrug resistant Escherichia species.
109. The method of embodiment 108, wherein the multi-drug resistant Escherichia species is resistant to at least one antibiotic.
110. The method of any one of embodiments 101 or 109, wherein the antibiotic comprises a cephalosporin, a fluoroquinolone, a carbapenem, a colistin, an aminoglycoside, vancomycin, streptomycin, or methicillin.
111. The method of any one of embodiments 76-110, wherein the Escherichia species is Escherichia coil.
112. The method of any one of embodiments 76-111, wherein the administering is intra-arterial, intravenous, intraurethral, intramuscular, oral, subcutaneous, inhalation, topical, cutaneous, transdermal, transmucosal, implantation, sublingual, buccal, rectal, vaginal, ocular, otic, or nasal administration or any combination thereof.
113. The method of any one of embodiments 76-112, wherein the individual is a mammal.
114. The method of any one of embodiments 76-113, further comprising administering at least one additional bacteriophage.
115. A bacteriophage comprising a nucleic acid sequence encoding a Type I CRISPR-Cas system comprising:
   (a) a CRISPR array comprising a spacer sequence complementary to target nucleotide sequence in a Escherichia species;
   (b) a Cascade polypeptide comprising Cas5, Cas8c and Cas7; and
   (c) a Cas3 polypeptide.
116. The bacteriophage of embodiment 115, wherein the spacer sequence comprises at least about 90%, 91%, 92%, 93%, 94%, 95%, 96%, 97%, 98%, or 99% sequence identity to any one of SEQ ID NOS: 12 or 20-37.
117. The bacteriophage of embodiment 115 or embodiment 116, wherein the spacer sequence comprises a first spacer sequence and a second spacer sequence, wherein the first spacer sequence is complementary to the target nucleotide sequence in the Escherichia species, and the second spacer sequence is complementary to a second target nucleotide sequence in the Escherichia species and/or a second Escherichia species; optionally wherein the spacer sequence comprises a third spacer sequence, wherein the third spacer sequence is complementary to a third target nucleotide sequence in the Escherichia species, second Escherichia species, and/or third Escherichia species.
118. The bacteriophage of any one of embodiments 115-117, wherein the CRISPR array further comprises at least one repeat sequence, wherein optionally the at least one repeat sequence is operably linked to the spacer sequence at either its 5′ end or its 3′ end.
119. The bacteriophage of embodiment 118, wherein the repeat sequence comprises at least about 90%, 91%, 92%, 93%, 94%, 95%, 96%, 97%, 98%, or 99% sequence identity to any one of SEQ ID NOs: 13-18.
120. The bacteriophage of any one of embodiments 115-119, wherein the CRISPR array comprises at least about 90%, 91%, 92%, 93%, 94%, 95%, 96%, 97%, 98%, or 99% sequence identity to a sequence selected from SEQ ID NOS: 38-45.
121. The bacteriophage of any one of embodiments 115-120, wherein the target nucleotide sequence comprises a coding sequence.
122. The bacteriophage of any one of embodiments 115-120, wherein the target nucleotide sequence comprises a non-coding or intergenic sequence.
123. The bacteriophage of any one of embodiments 115-122, wherein the target nucleotide sequence comprises all or a part of (e.g., at least 10 contiguous nucleobases) a promoter sequence.
124. The bacteriophage of embodiment 123, wherein the promoter sequence comprises at least about 90%, 91%, 92%, 93%, 94%, 95%, 96%, 97%, 98%, or 99% sequence identity to any one of SEQ ID NOs: 1-11 or 19.
125. The bacteriophage of any one of embodiments 115-124, wherein the target nucleotide sequence comprises all or a part of (e.g., at least 10 contiguous nucleobases) a nucleotide sequence located on a coding strand of a transcribed region of an essential gene, or a non-essential gene.
126. The bacteriophage of embodiment 125, wherein the target nucleotide sequence comprises all of a part of (e.g., at least 10 contiguous nucleobases) a nucleotide sequence located on a coding strand of a transcribed region of an essential gene, and the essential gene is Tsf acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK.
127. The bacteriophage of any one of embodiments 115-126, wherein the CRISPR-Cas system further comprises a promoter sequence.
128. The bacteriophage of any one of embodiments 115-127, wherein the bacteriophage is an obligate lytic bacteriophage.
129. The bacteriophage of any one of embodiments 115-127, wherein the bacteriophage is a temperate bacteriophage that is rendered lytic.
130. The bacteriophage of embodiment 129, wherein the temperate bacteriophage is rendered lytic by the removal, replacement, or inactivation of a lysogeny gene.
131. The bacteriophage of any one of embodiments 127-130, wherein the Escherichia species is killed solely by lytic activity of the bacteriophage.
132. The bacteriophage of any one of embodiments 127-130, wherein the Escherichia species is killed solely by activity of the CRISPR system.
133. The bacteriophage of any one of embodiments 127-132, wherein the Escherichia species is killed by lytic activity of the bacteriophage in combination with activity of the CRISPR system.
134. The bacteriophage of embodiment 133, wherein the Escherichia species is killed by the activity of the CRISPR system, independently of the lytic activity of the bacteriophage.
135. The bacteriophage of embodiment 133, wherein the activity of the CRISPR system supplements or enhances the lytic activity of the bacteriophage.
136. The bacteriophage of embodiment 133, wherein the lytic activity of the bacteriophage and the activity of the CRISPR system are synergistic.
137. The bacteriophage of any one of embodiments 131-136, wherein the lytic activity of the bacteriophage, the activity of the CRISPR system, or both, is modulated by a concentration of the bacteriophage
138. The bacteriophage of any one of embodiments 115-137, wherein the bacteriophage infects multiple bacterial strains.
139. The bacteriophage of any one of embodiments 115-138, wherein the bacteriophage comprises at least 80% identity to p004ke007, p004Ke005, p004K, p00c0, p00ex, p00jc, p00ke, or p5516.
140. The bacteriophage of any one of embodiments 115-139, wherein the nucleic acid sequence is inserted into a non-essential bacteriophage gene.
141. A pharmaceutical composition comprising:
(a) the bacteriophage of any one of embodiments 115-140; and
(b) a pharmaceutically acceptable excipient.
142. The pharmaceutical composition of embodiment 141, wherein the pharmaceutical composition comprises at least two bacteriophage.
143. The pharmaceutical composition of embodiment 142, wherein the pharmaceutical composition comprises at last six bacteriophage, wherein each bacteriophage comprises at least 80% sequence identity to p004k, p00c0, p00ex, p00jc, p00ke, and p5516; or p00exe014, p004Ke009, p00jc, p00ke, p00c0e030, p00c0e103, and p5516.
144. The pharmaceutical composition of any one of embodiments 141-143, wherein the pharmaceutical composition is in the form of a tablet, a capsule, a liquid, a syrup, an oral formulation, an intravenous formulation, an intranasal formulation, an ocular formulation, an otic formulation, a subcutaneous formulation, a topical formulation, a transdermal formulation, a transmucosal formulation, an inhalable respiratory formulation, a suppository, a lyophilized formulation, a nebulizable formulation, and any combination thereof.
145. A method of sanitizing a surface in need thereof, the method comprising administering to the surface a bacteriophage comprising a Type I CRISPR system comprising:
   (a) a CRISPR array comprising a spacer sequence complementary to target nucleotide sequence in an Escherichia species; and
   (b) a nucleic acid sequence encoding a CRISPR associated nuclease.
146. The method of embodiment 145, wherein the surface is a hospital surface, a vehicle surface, an equipment surface, or an industrial surface.
147. The method of embodiment 145 or embodiment 146, wherein the CRISPR system comprises a CRISPR-Cpf1 system, a Type II CRISPR-Cas system, or a Type V CRISPR-Cas system.
148. The method of any one of embodiments 145-147, wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
149. The method of embodiment 148, wherein the Cascade complex comprises:
(i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (ii) a Cas6b polypeptide, a Cas8b polypeptide, a Cas7 polypeptide, and a Cas5 polypeptide (Type I-B CRISPR-Cas system); (iii) a Cas5d polypeptide, a Cas8c polypeptide, and a Cas7 polypeptide (Type I-C CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
150. The method of embodiment 149, wherein the Cascade complex comprises a Cas5d polypeptide, a Cas8c polypeptide, and a Cas7 polypeptide (Type I-C CRISPR-Cas system).
151. A method of preventing contamination in a food product or a nutritional supplement, the method comprising adding to the food product or the nutritional supplement a bacteriophage comprising a nucleic acid sequence encoding a Type I CRISPR system comprising:
(a) a CRISPR array comprising a spacer sequence complementary to target nucleotide sequence in an Escherichia species; and
(b) a nucleic acid sequence encoding a CRISPR associated nuclease.
152. The method of embodiment 151, wherein the food product or nutritional supplement comprises milk, yoghurt, curd, cheese, fermented milks, milk based fermented products, ice-creams, fermented cereal based products, milk based powders, infant formulae or tablets, liquid suspensions, dried oral supplement, wet oral supplement, or dry-tube-feeding.
153. The method of embodiment 151 or embodiment 152, wherein the CRISPR system comprises a CRISPR-Cpf1 system, a Type II CRISPR-Cas system, or a Type V CRISPR-Cas system.
154. The method of any one of embodiments 151-153 wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
155. The method of embodiment 154, wherein the Cascade complex comprises:
(i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (ii) a Cas6b polypeptide, a Cas8b polypeptide, a Cas7 polypeptide, and a Cas5 polypeptide (Type I-B CRISPR-Cas system); (iii) a Cas5d polypeptide, a Cas8c polypeptide, and a Cas7 polypeptide (Type I-C CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
156. The method of embodiment 155, wherein the Cascade complex comprises a Cas5d polypeptide, a Cas8c polypeptide, and a Cas7 polypeptide (Type I-C CRISPR-Cas system).
157. A bacteriophage comprising a nucleic acid sequence encoding a Type I CRISPR system comprising:
(a) a CRISPR array comprising spacer sequences complementary to target nucleotide sequence in a Escherichia species, wherein the spacer sequences comprise at least one of SEQ ID NOS: 12 or 20-37; and
(b) a nucleic acid sequence encoding a CRISPR associated nuclease.
158. The bacteriophage of embodiment 157, wherein the spacer sequences comprise a first spacer sequence and a second spacer sequence, wherein the first spacer sequence is complementary to the target nucleotide sequence in the Escherichia species, and the second spacer sequence is complementary to a second target nucleotide sequence in the Escherichia species and/or a second Escherichia species; optionally wherein the spacer sequence comprises a third spacer sequence, wherein the third spacer sequence is complementary to a third target nucleotide sequence in the Escherichia species, second Escherichia species, and/or third Escherichia species.
159. The bacteriophage of embodiment 157 or embodiment 158, wherein the spacer sequences comprise two or three sequences selected from SEQ ID NOS: 12 or 20-37.
160. The bacteriophage of any one of embodiments 157-159, wherein the CRISPR system comprises a CRISPR-Cpf1 system, a Type II CRISPR-Cas system, or a Type V CRISPR-Cas system.
161. The bacteriophage of any one of embodiments 157-159, wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
162. The bacteriophage of embodiment 161, wherein the Cascade complex comprises:
(i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (ii) a Cas6b polypeptide, a Cas8b polypeptide, a Cas7 polypeptide, and a Cas5 polypeptide (Type I-B CRISPR-Cas system); (iii) a Cas5d polypeptide, a Cas8c polypeptide, and a Cas7 polypeptide (Type I-C CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
163. The bacteriophage of embodiment 162, wherein the Cascade complex comprises a Cas5d polypeptide, a Cas8c polypeptide, and a Cas7 polypeptide (Type I-C CRISPR-Cas system).
164. A bacteriophage comprising at least 80% sequence identity to a bacteriophage selected from p004K, p00c0, p00ex, p00jc, p00ke, or p5516.
165. The bacteriophage of embodiment 164, wherein the bacteriophage comprises at least 80% identity to p004Ke007, p004Ke009, p00c0e030, p00c0e103, or p00exe014.
166. The bacteriophage of embodiment 164 or embodiment 165, further comprising
(a) a CRISPR array comprising one or more spacer sequences complementary to target nucleotide sequence in a Escherichia species;
(b) a nucleic acid encoding a Cascade polypeptide; and
(c) a nucleic acid encoding a Cas3 polypeptide.
167. The bacteriophage of embodiment 166, wherein the one or more spacer sequence comprises at least one of SEQ ID NOs: 12 or 20-37 or at least 90%, 91%, 92%, 93%, 94%, 95%, 96%, 97%, 98%, or 99% sequence identity to any one of SEQ ID NOs: 12 or 20-37.
168. The bacteriophage of embodiment 164, wherein the one or more spacer sequences comprise two or three spacer sequences, wherein each spacer sequence has at least about 90%, 91%, 92%, 93%, 94%, 95%, 96%, 97%, 98%, or 99% sequence identity to any one of SEQ ID NOS: 12 or 20-37.
169. The bacteriophage of any one of embodiments 166-168, wherein the one or more spacer sequences comprise a first spacer sequence and a second spacer sequence, wherein the first spacer sequence is complementary to the target nucleotide sequence in the Escherichia species, and the second spacer sequence is complementary to a second target nucleotide sequence in the Escherichia species and/or a second Escherichia species; optionally wherein the spacer sequence comprises a third spacer sequence, wherein the third spacer sequence is complementary to a third target nucleotide sequence in the Escherichia species, second Escherichia species, and/or third Escherichia species.
170. The bacteriophage of any one of embodiments 164-169, wherein the CRISPR array further comprises at least one repeat sequence.
171. The bacteriophage of embodiment 170, wherein the at least one repeat sequence is operably linked to the spacer sequence at either its 5′ end or its 3′ end.
172. The bacteriophage of embodiment 164 or embodiment 171, wherein the repeat sequence comprises at least about 90%, 91%, 92%, 93%, 94%, 95%, 96%, 97%, 98%, or 99% sequence identity to any one of SEQ ID NOs: 13-18.
173. The bacteriophage of any one of embodiments 164-172, wherein the CRISPR array comprises at least about 90%, 91%, 92%, 93%, 94%, 95%, 96%, 97%, 98%, or 99% sequence identity to a sequence selected from SEQ ID NOS: 38-45.
174. The bacteriophage of any one of embodiments 164-173, wherein the target nucleotide sequence comprises a coding sequence.
175. The bacteriophage of any one of embodiments 164-173, wherein the target nucleotide sequence comprises a non-coding or intergenic sequence.
176. The bacteriophage of any one of embodiments 164-174, wherein the comprising a promoter sequence.
177. The bacteriophage of embodiment 176, wherein the promoter sequence comprises at least about 90%, 91%, 92%, 93%, 94%, 95%, 96%, 97%, 98%, or 99% sequence identity to any one of SEQ ID NOs: 1-11 or 19.
178. A bacteriophage modified to replace bacteriophage DNA with a nucleic acid sequence encoding a CRISPR system comprising: a CRISPR array comprising a spacer sequence complementary to a target nucleotide sequence in a target bacteria, and a sequence encoding a CRISPR nuclease.
179. The bacteriophage of embodiment 178, wherein the target bacteria comprises an Escherichia species.
180. The bacteriophage of embodiment 178 or embodiment 179, wherein the modified bacteriophage is viable.
181. The bacteriophage of any one of embodiments 178-180, wherein the modified bacteriophage is lytic.
182. The bacteriophage of any one of embodiments 178-181, wherein the nucleic acid sequence encoding the CRISPR system is a single nucleic acid sequence.
183. The bacteriophage of any one of embodiments 178-181, wherein the nucleic acid sequence encoding the CRISPR system comprises two or more nucleic acid sequences encoding the CRISPR system.
184. The bacteriophage of any one of embodiments 178-183, wherein the CRISPR system, CRISPR array, and/or sequence encoding the CRISPR nuclease is positioned adjacent to (e.g., 0 nucleobases away) or at least one nucleobase away from a bacteriophage coding sequence comprising a promoter and a gene encoding a protein.
185. The bacteriophage of any one of embodiments 178-184, wherein the CRISPR system, CRISPR array, and/or sequence encoding the CRISPR nuclease is positioned adjacent to (e.g., 0 nucleobases away) or at least one nucleobase away from an essential gene of the bacteriophage.
186. The bacteriophage of any one of embodiments 178-185, wherein the CRISPR system comprises a total of about 3000 nucleobases to about 8000 nucleobases.
187. The bacteriophage of any one of embodiments 178-186, wherein the CRISPR system comprises a total of about 4000 nucleobases to about 8000 nucleobases.
188. The bacteriophage of any one of embodiments 178-187, wherein the CRISPR system comprises a total of about 5000 nucleobases to about 8000 nucleobases.
189. The bacteriophage of any one of embodiments 178-188, wherein the bacteriophage DNA is from a p004ke, p00c0, or p00ex bacteriophage.
190. The bacteriophage of any one of embodiments 178-189, wherein the bacteriophage is a modified p00ke bacteriophage.
191. The bacteriophage of any one of embodiments 178-189, wherein the bacteriophage is a modified p00c0 bacteriophage.
192. The bacteriophage of any one of embodiments 178-189, wherein the bacteriophage is a modified p00ex bacteriophage.
193. The bacteriophage of any one of embodiments 178-192, wherein the bacteriophage DNA is not essential for viability, does not affect bacteriophage replication, does not affect bacteriophage lysis, does not affect the natural lifestyle of the bacteriophage, or does not affect the functionality of the bacteriophage, or any combination of two or more thereof.
194. The bacteriophage of any one of embodiments 178-193, wherein the CRISPR system is a CRISPR-Cas system.
195. The bacteriophage of any one of embodiments 178-193, wherein the CRISPR system is a CRISPR-Cpf1 system.
196. The bacteriophage of any one of embodiments 178-193, wherein the CRISPR system comprises a CRISPR-Cpf1 system, a Type II CRISPR-Cas system, or a Type V CRISPR-Cas system.
197. The bacteriophage of any one of embodiments 178-193, wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
198. The bacteriophage of embodiment 197, wherein the Cascade complex comprises: (i) a Cas5d polypeptide, a Cas8c polypeptide, and a Cas7 polypeptide (Type I-C CRISPR-Cas system); (ii) a Cas7 polypeptide, a Cas8a1 polypeptide or a Cas8a2 polypeptide, a Cas5 polypeptide, a Csa5 polypeptide, and a Cas6a polypeptide, a Cas3′ polypeptide, and a Cas3″ polypeptide having no nuclease activity (Type I-A CRISPR-Cas system); (iii) a Cas6b polypeptide, a Cas8b polypeptide, a Cas7 polypeptide, and a Cas5 polypeptide (Type I-B CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
199. The bacteriophage of embodiment 198, wherein the Cascade complex comprises a Cas5d polypeptide, a Cas8c polypeptide, and a Cas7 polypeptide (Type I-C CRISPR-Cas system).
200. The bacteriophage of embodiment 198, wherein the Cascade complex comprises a Pseudomonas aeruginosa Type I-C Cascade complex.
201. The bacteriophage of any one of embodiments 178-193, wherein the CRISPR system comprises Type I CRISPR-Cas components.
202. The bacteriophage of any one of embodiments 178-193, wherein the CRISPR system comprises Type V CRISPR-Cas components.
203. The bacteriophage of any one of embodiments 178-193, wherein the CRISPR system comprises Type II CRISPR-Cas components.
204. The bacteriophage of any one of embodiments 178-193, wherein the CRISPR nuclease comprises a Cas3, or a Cas3′ and a Cas3″ having no nuclease activity.
205. The bacteriophage of any one of embodiments 178-193, wherein the CRISPR nuclease comprises Cpf1.
206. The bacteriophage of any one of embodiments 178-193, wherein the CRISPR nuclease comprises Cas9.
207. The bacteriophage of any one of embodiments 178-206, wherein the spacer sequence comprises at least about 90%, 91%, 92%, 93%, 94%, 95%, 96%, 97%, 98%, or 99% sequence identity to any one of SEQ ID NOS: 12 or 20-37.
208. The bacteriophage of any one of embodiments 178-207, wherein the CRISPR array further comprises at least one repeat sequence.
209. The bacteriophage of embodiment 208, wherein the at least one repeat sequence is operably linked to the spacer sequence at either its 5′ end or its 3′ end.
210. The bacteriophage of embodiment 208 or embodiment 209, wherein the repeat sequence comprises at least about 90%, 91%, 92%, 93%, 94%, 95%, 96%, 97%, 98%, or 99% sequence identity to any one of SEQ ID NOs: 13-18.
211. The bacteriophage of any one of embodiments 178-210, wherein the CRISPR array comprises at least about 90%, 91%, 92%, 93%, 94%, 95%, 96%, 97%, 98%, or 99% sequence identity to a sequence selected from SEQ ID NOS: 38-45.
212. The bacteriophage of any one of embodiments 178-211, wherein the target nucleotide sequence comprises a coding sequence.
213. The bacteriophage of any one of embodiments 178-211, wherein the target nucleotide sequence comprises a non-coding or intergenic sequence.
214. The bacteriophage of any one of embodiments 178-213, wherein the target nucleotide sequence comprises all or a part of (e.g., at least 10 contiguous nucleobases) a promoter sequence.
215. The bacteriophage of embodiment 214, wherein the promoter sequence comprises at least about 90%, 91%, 92%, 93%, 94%, 95%, 96%, 97%, 98%, or 99% sequence identity to any one of SEQ ID NOs: 1-11 or 19.
216. The bacteriophage of any one of embodiments 178-215, wherein the target nucleotide sequence comprises all or a part of (e.g., at least 10 contiguous nucleobases) a nucleotide sequence located on a coding strand of a transcribed region of an essential gene, or a non-essential gene.
217. The bacteriophage of embodiment 216, wherein the target nucleotide sequence comprises all of a part of (e.g., at least 10 contiguous nucleobases) a nucleotide sequence located on a coding strand of a transcribed region of an essential gene, and the essential gene is Tsf acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK.
218. The bacteriophage of any one of embodiments 178-217, wherein the CRISPR system comprises a nucleic acid sequence comprising a promoter sequence.
219. The bacteriophage of any one of embodiments 178-218, wherein the bacteriophage infects multiple bacterial strains.
220. The bacteriophage of any one of embodiments 178-219, wherein the bacteriophage is an obligate lytic bacteriophage.
221. The bacteriophage of any one of embodiments 178-219, wherein the bacteriophage is a temperate bacteriophage that is rendered lytic.
222. The bacteriophage of embodiment 221, wherein the temperate bacteriophage is rendered lytic by the removal, replacement, or inactivation of a lysogeny gene.
223. The bacteriophage of any one of embodiments 178-222, wherein the target bacteria is killed solely by lytic activity of the bacteriophage.
224. The bacteriophage of any one of embodiments 178-222, wherein the target bacteria is killed solely by activity of the CRISPR system.
225. The bacteriophage of any one of embodiments 178-222, wherein the target bacteria is killed by lytic activity of the bacteriophage in combination with activity of the CRISPR system.
226. The bacteriophage of embodiment 220 or embodiment 221, wherein the target bacteria is killed by the activity of the CRISPR system, independently of the lytic activity of the bacteriophage.
227. The bacteriophage of embodiment 220 or embodiment 221, wherein the activity of the CRISPR system supplements or enhances the lytic activity of the bacteriophage.
228. The bacteriophage of embodiment 220 or embodiment 221, wherein the lytic activity of the bacteriophage and the activity of the CRISPR system are synergistic.
229. The bacteriophage of embodiment 220 or embodiment 221, wherein the lytic activity of the bacteriophage, the activity of the CRISPR system, or both, is modulated by a concentration of the bacteriophage.
230. The bacteriophage of any one of embodiments 178-229, wherein the bacteriophage comprises a Tequatrovirus, a Mosigyvirus, a Phapecoctavirus, a Unique Myoviridae, or a Vectrevirus.
231. The bacteriophage of any one of embodiments 178-230, wherein the bacteriophage comprises at least 80% sequence identity to a bacteriophage selected from p004K, p00c0, p00ex, p00jc, p00ke, p5516, p00exe014, p004Ke009, p00c0e030, or p00c0e103.
232. The bacteriophage of any one of embodiments 178-231, wherein the bacteriophage is modified from a bacteriophage selected from p004K, p00c0, p00ex, p00jc, p00ke, or p5516.
233. A nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOS: 38-45.
234. A nucleic acid sequence having at least 80% identity to SEQ ID NO: 39.
235. The nucleic acid of embodiment 234, having at least 90%, 91%, 92%, 93%, 94%, 95%, 96%, 97%, 98%, or 99% sequence identity to SEQ ID NO: 39.
236. The nucleic acid of embodiment 234, comprising SEQ ID NO: 39.
237. A nucleic acid sequence having at least 80% identity to SEQ ID NO: 43.
238. The nucleic acid of embodiment 237, having at least 90%, 91%, 92%, 93%, 94%, 95%, 96%, 97%, 98%, or 99% sequence identity to SEQ ID NO: 43.
239. The nucleic acid of embodiment 237, comprising SEQ ID NO: 43.
240. The nucleic acid of any one of embodiments 233-239, comprising SEQ ID NO: 12.
241. The nucleic acid of any one of embodiments 233-240, comprising SEQ ID NO: 25.
242. The nucleic acid of any one of embodiments 233-241, comprising SEQ ID NO: 24.
243. The nucleic acid of any one of embodiments 233-242, comprising SEQ ID NO: 13.
244. A CRISPR array comprising the nucleic acid sequence of any one of embodiments 227-243.
245. The CRISPR array of embodiment 244, further comprising a promoter.
246. The CRISPR array of embodiment 245, wherein the promoter is a promoter from Escherichia, Shigella, Klebsiella, Pseudomonas, or other bacterial species.
247. The CRISPR array of embodiment 245 or embodiment 246, wherein the promoter comprises a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOS: 1-11 or 19.
248. The CRISPR array of embodiment 245 or embodiment 246, wherein the promoter comprises a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 11.
249. The CRISPR array of any one of embodiments 245-248, wherein the promoter is a rrnB P1 promoter.
250. The CRISPR array of any one of embodiments 233-249, comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 43.
251. A bacteriophage comprising the nucleic acid of any one of embodiments 233-243, or the CRISPR array of any one of embodiments 244-250.
252. The bacteriophage of embodiment 251, wherein the nucleic acid and/or CRISPR array is positioned adjacent to (e.g., 0 nucleobases away) or at least one nucleobase away from a bacteriophage coding sequence comprising a promoter and a gene encoding a protein.
253. The bacteriophage of embodiment 251 or embodiment 252, wherein the nucleic acid and/or CRISPR array is positioned adjacent to (e.g., 0 nucleobases away) or at least one nucleobase away from a bacteriophage coding sequence comprising a promoter and a gene encoding a protein.
254. The bacteriophage of any one of embodiments 251-253, wherein the nucleic acid and/or CRISPR array replaces bacteriophage DNA.
255. The bacteriophage of embodiment 254, wherein the bacteriophage DNA is from a p004ke, p00c0, or p00ex bacteriophage.
256. The bacteriophage of embodiment 254 or embodiment 255, wherein the bacteriophage DNA is a modified p00ke bacteriophage.
257. The bacteriophage of embodiment 254 or embodiment 256, wherein the bacteriophage DNA is a modified p00c0 bacteriophage.
258. The bacteriophage of embodiment 254 or embodiment 256, wherein the bacteriophage DNA is a modified p00ex or bacteriophage.
259. The bacteriophage of any one of embodiments 251-258, wherein the bacteriophage DNA is not essential for viability, does not affect bacteriophage replication, does not affect bacteriophage lysis, does not affect the natural lifestyle of the bacteriophage, or does not affect the functionality of the bacteriophage, or any combination of two or more thereof.
260. The bacteriophage of any one of embodiments 251-259, wherein the bacteriophage is viable.
261. The bacteriophage of any one of embodiments 251-260, wherein the bacteriophage infects multiple bacterial strains.
262. The bacteriophage of any one of embodiments 251-261, wherein the bacteriophage is lytic.
263. The bacteriophage of any one of embodiments 251-261, wherein the bacteriophage is an obligate lytic bacteriophage.
264. The bacteriophage of any one of embodiments 251-263, wherein the bacteriophage is a temperate bacteriophage that is rendered lytic.
265. The bacteriophage of embodiment 264, wherein the temperate bacteriophage is rendered lytic by the removal, replacement, or inactivation of a lysogeny gene.
266. The bacteriophage of any one of embodiments 251-265, wherein the bacteriophage comprises a Tequatrovirus, a Mosigyvirus, a Phapecoctavirus, a Unique Myoviridae, or a Vectrevirus.
267. The bacteriophage of any one of embodiments 251-266, wherein the nucleic acid and/or CRISPR array is part of a CRISPR system present in the bacteriophage.
268. The bacteriophage of embodiment 267, wherein the CRISPR system comprises a total of about 3000 nucleobases to about 8000 nucleobases.
269. The bacteriophage of embodiment 267, wherein the CRISPR system comprises a total of about 4000 nucleobases to about 8000 nucleobases.
270. The bacteriophage of embodiment 267, wherein the CRISPR system comprises a total of about 5000 nucleobases to about 8000 nucleobases.
271. The bacteriophage of any one of embodiments 267-270, wherein the CRISPR system is a CRISPR-Cas system.
272. The bacteriophage of any one of embodiments 267-270, wherein the CRISPR system is a CRISPR-Cpf1 system.
273. The bacteriophage of any one of embodiments 267-270, wherein the CRISPR system comprises a CRISPR-Cpf1 system, a Type II CRISPR-Cas system, or a Type V CRISPR-Cas system.
274. The bacteriophage of any one of embodiments 267-270, wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
275. The bacteriophage of embodiment 274, wherein the Cascade complex comprises:
(i) a Cas5d polypeptide, a Cas8c polypeptide, and a Cas7 polypeptide (Type I-C CRISPR-Cas system); (ii) a Cas7 polypeptide, a Cas8a1 polypeptide or a Cas8a2 polypeptide, a Cas5 polypeptide, a Csa5 polypeptide, and a Cas6a polypeptide, a Cas3′ polypeptide, and a Cas3″ polypeptide having no nuclease activity (Type I-A CRISPR-Cas system); (iii) a Cas6b polypeptide, a Cas8b polypeptide, a Cas7 polypeptide, and a Cas5 polypeptide (Type I-B CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
276. The bacteriophage of embodiment 275, wherein the Cascade complex comprises a Cas5d polypeptide, a Cas8c polypeptide, and a Cas7 polypeptide (Type I-C CRISPR-Cas system).
277. The bacteriophage of embodiment 266 or embodiment 267, wherein the Cascade complex comprises a Pseudomonas aeruginosa Type I-C Cascade complex.
278. The bacteriophage of any one of embodiments 267-270, wherein the CRISPR system comprises Type I CRISPR-Cas components.
279. The bacteriophage of any one of embodiments 267-270, wherein the CRISPR system comprises Type V CRISPR-Cas components.
280. The bacteriophage of any one of embodiments 267-270, wherein the CRISPR system comprises Type II CRISPR-Cas components.
281. The bacteriophage of any one of embodiments 267-270, wherein the CRISPR system comprises a nucleic acid encoding a nuclease.
282. The bacteriophage of embodiment 281, wherein the nuclease comprises a Cas 3, or a Cas3′ and a Cas3″ having no nuclease activity.
283. The bacteriophage of embodiment 281, wherein the nuclease comprises Cpf1.
284. The bacteriophage of embodiment 281, wherein the CRISPR nuclease comprises Cas9.
285. The bacteriophage of any one of embodiments 251-284, wherein the bacteriophage comprises at least 80%, 81%, 82%, 83%, 84%, 85%, 86%, 87%, 88%, 89%, 90%, 91%, 92%, 93%, 94%, 95%, 96%, 97%, 98%, or 99% sequence identity to a bacteriophage selected from p004K, p00c0, p00ex, p00jc, p00ke, or p5516.
286. The bacteriophage of any one of embodiments 251-285, wherein the bacteriophage is modified from a bacteriophage selected from p004K, p00c0, p00ex, p00jc, p00ke, or p5516.
287. The bacteriophage of any one of embodiments 251-286, wherein the bacteriophage comprises at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004Ke009.
288. The bacteriophage of any one of embodiments 251-286, wherein the bacteriophage comprises at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030.
289. The bacteriophage of any one of embodiments 251-286, wherein the bacteriophage comprises at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014.
290. A composition comprising the bacteriophage of any one of embodiments 1-232 or 251-289, and a wild-type bacteriophage.
291. The composition of embodiment 290, wherein the wild-type bacteriophage target any one of the bacteria of Table 6.
292. The composition of embodiment 290 or embodiment 291, wherein the wild-type bacteriophage comprises p00ke.
293. The composition of any one of embodiments 290-292, wherein the wild-type bacteriophage comprises p5516.
294. The composition of any one of embodiments 290-293, wherein the wild-type bacteriophage comprises p00jc.
295. A composition comprising the bacteriophage of any one of embodiments 1-232 or 251-289, and a second bacteriophage.
296. The composition of embodiment 295, wherein the second bacteriophage is from the lineage consisting of a Tequatrovirus, a Mosigyvirus, a Phapecoctavirus, a Unique Myoviridae, or a Vectrevirus.
297. The composition of embodiment 295 or embodiment 296, wherein the second bacteriophage is modified to replace bacteriophage DNA with a nucleic acid sequence encoding a CRISPR system comprising: a CRISPR array comprising a spacer sequence complementary to a target nucleotide sequence in a target bacteria, and a sequence encoding a CRISPR nuclease.
298. The composition of any one of embodiments 295-297, wherein at least one of the bacteria of Table 6 is targeted by the second bacteriophage.
299. The composition of embodiment 298, wherein at least 10, 20, 30, 40, 50, 100, 150, 200, 250, or 300 of the bacteria of Table 6 are targeted by the second bacteriophage.
300. The composition of any one of embodiments 295-299, wherein the second bacteriophage comprises at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009.
301. The composition of any one of embodiments 295-299, wherein the second bacteriophage comprises at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030.
302. The composition of any one of embodiments 295-299, wherein the second bacteriophage comprises at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014.
303. A recombinant bacteriophage comprising at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009.
304. A recombinant bacteriophage comprising at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030.
305. A recombinant bacteriophage comprising at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014.
306. A bacteriophage comprising at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00jc.
307. A bacteriophage comprising at least 80%, 81%, 82%, 83%, 84%, 85%, 86%, 87%, 88%, 89%,
90%, 91%, 92%, 93%, 94%, 95%, 96%, 97%, 98%, or 99% sequence identity to p00ke.
308. A bacteriophage comprising at least 80%, 81%, 82%, 83%, 84%, 85%, 86%, 87%, 88%, 89%, 90%, 91%, 92%, 93%, 94%, 95%, 96%, 97%, 98%, or 99% sequence identity to p5516.
309. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, and a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030, p00c0e103, p00exe014, p00jc, p00ke, or p5516.
310. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030, and a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, p00exe014, p00jc, p00ke, or p5516.
311. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014, and a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, p00c0e030, p00c0e103, p00jc, p00ke, or p5516.
312. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00jc, and a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, p00c0e030, p00c0e103, p00exe014, p00ke, or p5516.
313. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00ke, and a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, p00c0e030, p00c0e103, p00exe014, p00jc, or p5516.
314. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p5516, and a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, p00c0e030, p00c0e103, p00exe014, p00jc, or p00ke.
315. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030, p00c0e103, and a third bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00jc, p00ke, or p5516.
316. The composition of embodiment 315, comprising a fourth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00jc, p00ke, or p5516; optionally comprising a fifth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00jc, p00ke, or p5516; and optionally comprising a sixth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00jc, p00ke, or p5516.
317. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014, and a third bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00c0e030, p00c0e103, p00jc, p00ke, or p5516.
318. The composition of embodiment 317, comprising a fourth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00c0e030, p00c0e103, p00jc, p00ke, or p5516; optionally comprising a fifth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00c0e030, p00c0e103, p00jc, p00ke, or p5516; and optionally comprising a sixth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00c0e030, p00c0e103, p00jc, p00ke, or p5516.
319. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00jc, and a third bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00c0e103, p00ke, or p5516.
320. The composition of embodiment 319, comprising a fourth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00c0e103, p00ke, or p5516; optionally comprising a fifth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00c0e103, p00ke, or p5516; and optionally comprising a sixth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00c0e103, p00ke, or p5516.
321. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00ke, and a third bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00c0e103, p00jc, or p5516.
322. The composition of embodiment 321, comprising a fourth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00c0e103, p00jc, or p5516; optionally comprising a fifth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00c0e103, p00jc, or p5516; and optionally comprising a sixth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00c0e103, p00jc, or p5516.
323. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p5516, and a third bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00c0e103, p00ke, or p00jc.
324. The composition of embodiment 323, comprising a fourth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00c0e103, p00ke, or p00jc; optionally comprising a fifth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00c0e103, p00ke, or p00jc; and optionally comprising a sixth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00c0e103, p00ke, or p00jc.
325. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030, p00c0e103, a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014, and a third bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00jc.
326. The composition of embodiment 325, comprising a fourth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00jc; optionally comprising a fifth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00jc; and optionally comprising a sixth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00jc.
327. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030, p00c0e103, a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00jc, and a third bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00exe014.
328. The composition of embodiment 327, comprising a fourth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00exe014; optionally comprising a fifth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00exe014; and optionally comprising a sixth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00exe014.
329. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030, p00c0e103, a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00ke, and a third bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00exe014, or p00jc.
330. The composition of embodiment 329, comprising a fourth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00exe014, or p00jc; optionally comprising a fifth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00exe014, or p00jc; and optionally comprising a sixth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00exe014, or p00jc.
331. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030, p00c0e103, a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p5516, and a third bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00exe014, or p00jc.
332. The composition of embodiment 331, comprising a fourth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00exe014, or p00jc; optionally comprising a fifth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00exe014, or p00jc; and optionally comprising a sixth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00exe014, or p00jc.
333. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014, a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00jc, and a third bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00c0e030, p00c0e103, or p5516.
334. The composition of embodiment 333, comprising a fourth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00c0e030, p00c0e103, or p5516; optionally comprising a fifth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00c0e030, p00c0e103, or p5516; and optionally comprising a sixth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00c0e030, p00c0e103, or p5516.
335. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014, a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00ke, and a third bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, p00c0e103, or p5516.
336. The composition of embodiment 335, comprising a fourth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, p00c0e103, or p5516; optionally comprising a fifth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, p00c0e103, or p5516; and optionally comprising a sixth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, p00c0e103, or p5516.
337. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014, a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p5516, and a third bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, p00c0e103, or p00ke.
338. The composition of embodiment 337, comprising a fourth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, p00c0e103, or p00ke; optionally comprising a fifth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, p00c0e103, or p00ke; and optionally comprising a sixth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, p00c0e103, or p00ke.
339. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00jc, a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00ke, and a third bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, p00c0e103, or p5516.
340. The composition of embodiment 339, comprising a fourth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, p00c0e103, or p5516; optionally comprising a fifth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, p00c0e103, or p5516; and optionally comprising a sixth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, p00c0e103, or p5516.
341. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00jc, a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p5516, and a third bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, p00c0e103, or p00ke.
342. The composition of embodiment 341, comprising a fourth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, p00c0e103, or p00ke; optionally comprising a fifth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, p00c0e103, or p00ke; and optionally comprising a sixth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, p00c0e103, or p00ke.
343. A composition comprising a first bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00ke, a second bacteriophage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p5516, and a third bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, p00c0e103, or p00jc.
344. The composition of embodiment 343, comprising a fourth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, p00c0e103, or p00jc; optionally comprising a fifth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, p00c0e103, or p00jc; and optionally comprising a sixth bacteriophage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, p00c0e103, or p00jc.
345. The composition of any one of embodiments 316, 318, 320, 322, 324, 326, 328, 330, 332, 334, 336, 338, 340, 342 or 344, wherein the fourth bacteriophage is different from the third page.
346. The composition of any one of embodiments 316, 318, 320, 322, 324, 326, 328, 330, 332, 334, 336, 338, 340, 342, 344 or 345, wherein the fifth bacteriophage is present, and the fifth bacteriophage is different from the third and fourth bacteriophage.
347. The composition of any one of embodiments 316, 318, 320, 322, 324, 326, 328, 330, 332, 334, 336, 338, 340, 342 or 344-346, wherein the sixth bacteriophage is present, and the sixth bacteriophage is different from the third, fourth, and fifth bacteriophage.
348. A pharmaceutical composition comprising:
(a) the bacteriophage of any one of embodiments 1-232, 251-289, or 303-308 or the composition of any one of embodiments 290-302 or 309-347; and
(b) a pharmaceutically acceptable excipient.
349. The pharmaceutical composition of embodiment 339, wherein the pharmaceutical composition is in the form of a tablet, a capsule, a liquid, a syrup, an oral formulation, an intravenous formulation, an intranasal formulation, an ocular formulation, an otic formulation, a subcutaneous formulation, a topical formulation, a transdermal formulation, a transmucosal formulation, an inhalable respiratory formulation, a suppository, a lyophilized formulation, a nebulizable formulation, and any combination thereof.
350. A method of killing bacteria comprising introducing into the bacteria genetic material from the bacteriophage of any one of embodiments 1-232, 251-289, or 303-308.
351. A method of killing bacteria comprising introducing into the bacteria genetic material from a bacteriophage from the composition of any one of embodiments 290-302 or 309-347.
352. A method of treating a disease in an individual in need thereof, the method comprising administering to the individual the bacteriophage of any one of embodiments 1-232, 251-289, or 303-308.
353. A method of treating a disease in an individual in need thereof, the method comprising administering to the individual the composition of any one of embodiments 290-302 or 309-347.
354. The method of embodiment 352 or embodiment 353, wherein the disease is a bacterial infection.
355. The method of any one of embodiments 352-354, wherein the bacteria is Escherichia.
356. A method of sanitizing a surface in need thereof, the method comprising administering to the surface the bacteriophage of any one of embodiments 1-232, 251-289, or 303-308.
357. A method of sanitizing a surface in need thereof, the method comprising administering to the surface the composition of any one of embodiments 290-302 or 309-347.
358. The method of embodiment 356 or embodiment 357, wherein the surface comprises Escherichia.
359. A method of preventing bacterial contamination in a food product or a nutritional supplement, the method comprising adding to the food product or the nutritional supplement the bacteriophage of any one of embodiments 1-232, 251-289, or 303-308.
360. A method of preventing bacterial contamination in a food product or a nutritional supplement, the method comprising adding to the food product or the nutritional supplement the composition of any one of embodiments 290-302 or 309-347.
361. The method of embodiment 359 or embodiment 360, wherein the bacteria is Escherichia.
362. A method of killing bacteria comprising contacting the bacteria with a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00jc.
363. A method of killing bacteria comprising contacting the bacteria with a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00ke.
364. A method of killing bacteria comprising contacting the bacteria with a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p5516.
365. A method of killing bacteria comprising contacting the bacteria with a composition comprising p00jc.
366. A method of killing bacteria comprising contacting the bacteria with a composition comprising p00ke.
367. A method of killing bacteria comprising contacting the bacteria with a composition comprising p5516.
368. A method of treating a disease in an individual in need thereof, the method comprising administering to the individual a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00jc.
369. A method of treating a disease in an individual in need thereof, the method comprising administering to the individual a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00ke.
370. A method of treating a disease in an individual in need thereof, the method comprising administering to the individual a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p5516.
371. A method of treating a disease in an individual in need thereof, the method comprising administering to the individual a composition comprising p00jc.
372. A method of treating a disease in an individual in need thereof, the method comprising administering to the individual a composition comprising p00ke.
373. A method of treating a disease in an individual in need thereof, the method comprising administering to the individual a composition comprising p5516.
374. The method of any one of embodiments 359-364, wherein the disease is a bacterial infection.
375. A method of sanitizing a surface in need thereof, the method comprising administering to the surface a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00jc.
376. A method of sanitizing a surface in need thereof, the method comprising administering to the surface a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00ke.
377. A method of sanitizing a surface in need thereof, the method comprising administering to the surface a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p5516.
378. A method of sanitizing a surface in need thereof, the method comprising administering to the surface a composition comprising p00jc.
379. A method of sanitizing a surface in need thereof, the method comprising administering to the surface a composition comprising p00ke.
380. A method of sanitizing a surface in need thereof, the method comprising administering to the surface a composition comprising p5516.
381. A method of preventing bacterial contamination in a food product or a nutritional supplement, the method comprising adding to the food product or the nutritional supplement a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00jc.
382. A method of preventing bacterial contamination in a food product or a nutritional supplement, the method comprising adding to the food product or the nutritional supplement a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00ke.
383. A method of preventing bacterial contamination in a food product or a nutritional supplement, the method comprising adding to the food product or the nutritional supplement a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p5516.
384. A method of preventing bacterial contamination in a food product or a nutritional supplement, the method comprising adding to the food product or the nutritional supplement a composition comprising p00jc.
385. A method of preventing bacterial contamination in a food product or a nutritional supplement, the method comprising adding to the food product or the nutritional supplement a composition comprising p00ke.
386. A method of preventing bacterial contamination in a food product or a nutritional supplement, the method comprising adding to the food product or the nutritional supplement a composition comprising p5516.
387. The method of any one of embodiments 375-386, wherein the bacteria is Escherichia.
388. A method of killing a plurality of bacteria, the method comprising combining the plurality of bacteria with a first bacteriophage and a second bacteriophage, wherein the first bacteriophage targets a first subset of the plurality of bacteria, and the second bacteriophage targets the second subset of the plurality of bacteria.
389. A method of treating a disease in an individual comprising a plurality of bacteria, the method comprising administering to the individual a first bacteriophage and a second bacteriophage, wherein the first bacteriophage targets a first subset of the plurality of bacteria, and the second bacteriophage targets the second subset of the plurality of bacteria.
390. The method of embodiment 389, wherein the disease is a bacterial infection.
391. A method of sanitizing a surface comprising a plurality of bacteria, the method comprising administering to the surface a first bacteriophage and a second bacteriophage, wherein the first bacteriophage targets a first subset of the plurality of bacteria, and the second bacteriophage targets the second subset of the plurality of bacteria.
392. A method of preventing bacterial contamination from a plurality of bacteria in a food product or a nutritional supplement, the method comprising adding to the food product or the nutritional supplement a first bacteriophage and a second bacteriophage, wherein the first bacteriophage targets a first subset of the plurality of bacteria, and the second bacteriophage targets the second subset of the plurality of bacteria.
393. The method of any one of embodiments 388-392, wherein targeting the first subset of the plurality of bacteria comprises infecting the first subset of the plurality of bacteria, and targeting the second subset of the plurality of bacteria comprises infecting the second subset of the plurality of bacteria.
394. The method of any one of embodiments 388-393, wherein the first subset is different from the second subset.
395. The method of any one of embodiments 388-394, wherein the plurality of bacteria comprises two or more bacteria of Table 6.
396. The method of any one of embodiments 388-395, wherein the plurality of bacteria comprises at least 50, 100, 150, 200, 250, 300, or 350 bacteria of Table 6.
397. The method of any one of embodiments 388-396, wherein the first bacteriophage comprises the bacteriophage of any one of embodiments 1-232, 251-289, or 303-308.
398. The method of any one of embodiments 388-397, wherein the second bacteriophage comprises the bacteriophage of any one of embodiments 1-232, 251-289, or 303-308.
399. The method of any one of embodiments 388-396, wherein the first bacteriophage comprises a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identical to p00jc.
400. The method of any one of embodiments 388-396, wherein the first bacteriophage comprises a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identical to p00ke
401. The method of any one of embodiments 388-396, wherein the first bacteriophage comprises a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identical to p5516.
402. The method of any one of embodiments 388-396, wherein the first bacteriophage comprises a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identical to p004Ke009.
403. The method of any one of embodiments 388-396, wherein the first bacteriophage comprises a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identical to p00c0e030.
404. The method of any one of embodiments 388-396, wherein the first bacteriophage comprises a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identical to p00exe014.
405. The method of any one of embodiments 388-397 or 399-404, wherein the second bacteriophage comprises a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identical to p00jc.
406. The method of any one of embodiments 388-397 or 399-404, wherein the second bacteriophage comprises a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identical to p00ke.
407. The method of any one of embodiments 388-397 or 399-404, wherein the second bacteriophage comprises a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identical to p5516.
408. The method of any one of embodiments 388-397 or 399-404, wherein the second bacteriophage comprises a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identical to p004Ke009.
409. The method of any one of embodiments 388-397 or 399-404, wherein the second bacteriophage comprises a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identical to p00c0e030.
410. The method of any one of embodiments 388-397 or 399-404, wherein the second bacteriophage comprises a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identical to p00exe014.
411. A composition comprising one or more bacteriophage, wherein each of the one or more bacteriophage is present in the composition at a concentration of at least 1×10⁷ plaque forming units (PFU) per milliliter (ml) (PFU/ml), and wherein the composition has less than or equal to about 50 endotoxin units per ml.
412. The composition of embodiment 411, wherein the composition comprises at least 1×10⁷ PFU of total bacteriophages per ml.
413. The composition of embodiment 412, wherein the composition comprises from about 1×10⁷ PFU to about 1×10¹³ PFU of total bacteriophage per ml
414. The composition of any one of embodiments 411 to 413, wherein the composition comprises a total of about 1×10⁹-1×10¹⁴ PFU of total bacteriophages.
415. The composition of any one of embodiments 411 to 414, wherein each of the one or more bacteriophage is present in the composition from 1×10⁹-1×10¹⁴ PFU/mL
416. The composition of any one of embodiments 411 to 415, wherein the endotoxin level is determined by USP/NF<85>/DUR-QCT-1771-TM.
417. The composition of any one of embodiments 411 to 416, wherein the one or more bacteriophage comprises one or more of Tequatrovirus, Phapecoctavirus, Myovirus, Mosigvirus, and Vectrevirus.
418. The composition of embodiment 417, wherein the composition comprises a Tequatrovirus, and the Tequatrovirus is p00ex, p004k, or an engineered Tequatrovirus.
419. The composition of any one of embodiments 411 to 418, wherein at least one of the one or more bacteriophage comprises an exogenous CRISPR-Cas system component.
420. The composition of embodiment 419, wherein the CRISPR-Cas system is a Type I CRISPR Cas system.
421. The composition of any one of embodiments 411 to 420, wherein at least one of the one or more bacteriophage infect Escherichia.
422. A composition comprising one or more bacteriophage selected from Tequatrovirus, Phapecoctavirus, Myovirus, Mosigvirus, and Vectrevirus; wherein the composition has less than or equal to about 50 endotoxin units per ml.
423. A composition comprising one or more bacteriophage comprising a CRISPR-Cas system, wherein the composition has less than or equal to about 50 endotoxin units per ml.
424. The composition of any preceding embodiment, wherein the composition is administered to a subject in a dosage range between about 1×10⁹ and about 1×10¹⁴ PFU total bacteriophage.
425. The composition of any one of embodiments 422 to 424, wherein the composition is administered at least once a day for 3 days.
426. The composition of any one of embodiments 422 to 425, wherein the one or more bacteriophage target E. coli.
427. A method of treating a subject in need thereof, wherein the method comprises administering the composition of embodiments 411 to 426 to the subject.
428. The method of embodiment 427, wherein the subject is afflicted with an Escherichia infection.
429. The method of embodiment 427 or 428, wherein the Escherichia infection comprises a urinary tract infection.
430. The method of any one of embodiments 17 to 428, wherein the composition is administered intraurethrally, intravenously, or orally.
431. A method of treating a urinary tract infection in a subject in need thereof, the method comprising
administering a bacteriophage cocktail comprising at least a first bacteriophage and at least a second bacteriophage to a subject in need thereof;
wherein the recurrent urinary tract infection comprises a Escherichia coli infection, wherein the first bacteriophage and the second bacteriophage target E. coli.
432. The method of embodiment 431, wherein the urinary tract infection is a recurrent urinary tract infection.
433. A method of reducing the levels of Escherichia coli in a subject, the method comprising
administering a bacteriophage cocktail comprising at least a first bacteriophage and at least a second bacteriophage to a subject in need thereof, wherein the Escherichia coli concentration is or has been measured in a blood or urine sample of the patient.
434. The method of embodiment 433, wherein the Escherichia coli concentration is measured prior to administration of the bacteriophage cocktail.
435. The method of embodiment 433, wherein the Escherichia coli concentration is measured after administration of the bacteriophage cocktail.
436. The method of any one of embodiments 433 to 435, further comprising measuring the Escherichia coli concentration.
437. The method of any one of embodiments 431 to 436, wherein after administration of the bacteriophage cocktail, the subject does not have a reoccurrence of a urinary tract infection for at least three months.
438. The method of any one of embodiments 431 to 437, wherein the bacteriophage cocktail is administered intravenously.
439. The method of any one of embodiments 431 to 438, wherein the bacteriophage cocktail is administered orally.
440. The method of any one of embodiments 431 to 439, wherein the bacteriophage cocktail is administered intravenously on at least a first treatment day and wherein the bacteriophage cocktail is administered orally on at least a second treatment day.
441. The method of any one of embodiments 431 to 440, wherein the bacteriophage cocktail is administered orally for at least 3 days.
442. The method of any one of embodiments 431 to 441, wherein the bacteriophage cocktail is administered concurrently with an antibiotic.
443. The method of embodiment 442, wherein the antibiotic comprises sulfamethoxazole or trimethoprim.
444. The method of any one of embodiments 431 to 443, wherein the bacteriophage cocktail is administered at a concentration of 1×10¹⁰ to 1×10¹³ PFU.
445. The method of any one of embodiments 431 to 444, wherein the bacteriophage cocktail is administered with less than about 50 EU/mL endotoxin.
446. The method of any one of embodiments 431 to 445, wherein the first bacteriophage comprises comprising a CRISPR system comprising:
a. a CRISPR array comprising a first spacer sequence comprising a sequence selected from SEQ ID NOS: 12 or 20-37; and
b. a nucleic acid sequence encoding a CRISPR associated nuclease.
447. The method of embodiment 446, wherein the first spacer sequence comprises SEQ ID NO: 12, SEQ ID NO: 25, or SEQ ID NO: 24.
448. The method of embodiment 446 or 447, wherein the CRISPR array comprises a second spacer sequence.
449. The method of embodiment 448, wherein the first spacer sequence comprises SEQ ID NO: 12, and the second spacer sequence comprises SEQ ID NO: 25, or the first spacer sequence comprises SEQ ID NO: 12, and the second spacer sequence comprises SEQ ID NO: 24, or the first spacer sequence comprises SEQ ID NO: 25, and the second spacer sequence comprises SEQ ID NO: 24.
450. The method of any one of embodiments 446 to 449, wherein the CRISPR array comprises a third spacer sequence.
451. The method of embodiment 450, wherein the first spacer sequence comprises SEQ ID NO: 12, the second spacer sequence comprises SEQ ID NO: 25, and the third spacer sequence comprises SEQ ID NO: 24.
452. The method of any one of embodiments 431 to 451, wherein (i) the first bacteriophage comprises a phage bacteriophage at least 80% identical to p00jc, (ii) the first bacteriophage comprises a phage bacteriophage at least 80% identical to p00ke, (iii) the first bacteriophage comprises a phage bacteriophage at least 80% identical to p5516, (iv) the first bacteriophage comprises a phage bacteriophage at least 80% identical to p004Ke009, (v) the first bacteriophage comprises a phage at least 80% identical to p00c0e030, p00c0e103, or (vi) the first bacteriophage comprises a phage at least 80% identical to p00exe014.
453. The method of any one of embodiments 431 to 452, wherein (i) the first phage bacteriophage has at least 80% sequence identity to p004ke009, and the second phage bacteriophage has at least 80% sequence identity to p00c0e030, p00c0e103, p00exe014, p00jc, p00ke, or p5516, (ii) the first phage bacteriophage has at least 80% sequence identity to p00c0e030, p00c0e103, and the second phage has at least 80% sequence identity to p004ke009, p00exe014, p00jc, p00ke, or p5516, (iii) the first phage has at least 80% sequence identity to p00exe014, and the second phage has at least 80% sequence identity to p004ke009, p00c0e030, p00c0e103, p00jc, p00ke, or p5516, (iv) the first phage has at least 80% sequence identity to p00jc, and the second phage has at least 80% sequence identity to p004ke009, p00c0e030, p00c0e103, p00exe014, p00ke, or p5516, (v) the first phage has at least 80% sequence identity to p00ke, and the second phage has at least 80% sequence identity to p004ke009, p00c0e030, p00c0e103, p00exe014, p00jc, or p5516, or (vi) the first phage has at least 80% sequence identity to p5516, and the second phage has at least 80% sequence identity to p004ke009, p00c0e030, p00c0e103, p00exe014, p00jc, or p00ke.
454. The method of any one of embodiments 431 to 453, wherein the bacteriophage cocktail further comprises at least a third phage, wherein the third phage has at least 80% sequence identity to a phage selected from the list consisting of p00exe014, p004Ke009, p00jc, p00ke, p00c0e030, p00c0e103, and p5516.
455. The method of embodiment 454, wherein the bacteriophage cocktail further comprises at least a fourth phage, wherein the fourth phage has at least 80% sequence identity to a phage selected from the list consisting of p00exe014, p004Ke009, p00jc, p00ke, p00c0e030, p00c0e103, and p5516.
456. The method of embodiment 455, wherein the bacteriophage cocktail further comprises at least a fifth phage, wherein the fifth phage has at least 80% sequence identity to a phage selected from the list consisting of p00exe014, p004Ke009, p00jc, p00ke, p00c0e030, p00c0e103, and p5516.
457. The method of embodiment 456, wherein the bacteriophage cocktail further comprises at least a sixth phage, wherein the sixth phage has at least 80% sequence identity to a phage selected from the list consisting of p00exe014, p004Ke009, p00jc, p00ke, p00c0e030, p00c0e103, and p5516.
458. The method of embodiment 457, wherein the bacteriophage cocktail comprises p00exe014, p004Ke009, p00jc, p00ke, p00c0e030, p00c0e103, and p5516.
459. A composition comprising a plurality of bacteriophage that infect at least about 90% of a collection of at least 50 Escherichia isolates.
460. A composition comprising a plurality of bacteriophage that infect at least about 90% of the Escherichia isolates of Table 11.
461. The composition of embodiment 459 or 460, wherein the plurality of bacteriophage comprises one or more of Tequatrovirus, Phapecoctavirus, Myovirus, Mosigvirus, and Vectrevirus.
462. The composition of any one of embodiments 459 to 461, wherein the Escherichia isolates comprise pathogenic bacteria.
463. The composition of any one of embodiments 459 to 462, wherein the plurality of bacteriophage do not broadly target other Enterobacteriacea.
464. The composition of any one of embodiments 459 to 463, wherein the plurality of bacteriophage infect Escherichia coli and do not broadly target Shigella spp, Salmonella spp, Citrobacter spp, or a combination thereof.
465. The composition of any one of embodiments 459 to 464, wherein infectivity is determined with a plaque assay or growth inhibition assay.

### EXAMPLES

The following examples are included for illustrative purposes only and are not intended to limit the scope of the invention.

### Example 1: Engineered Bacteriophage Used in this Application

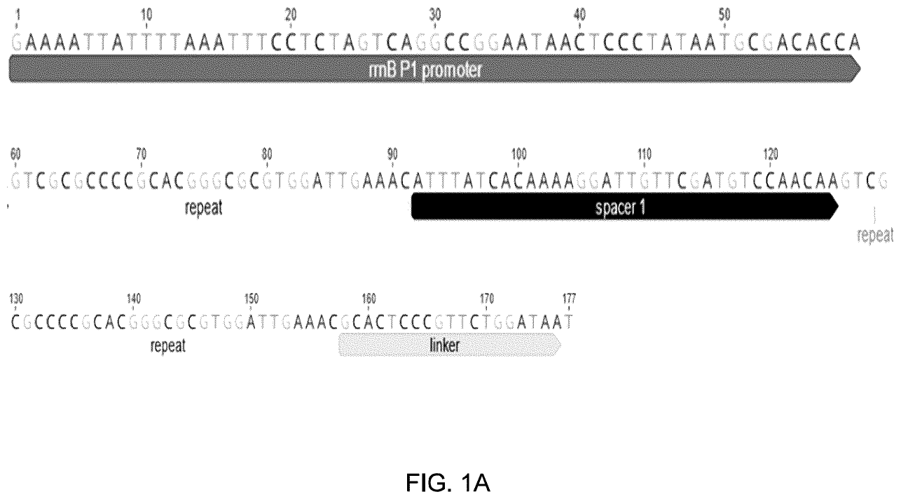

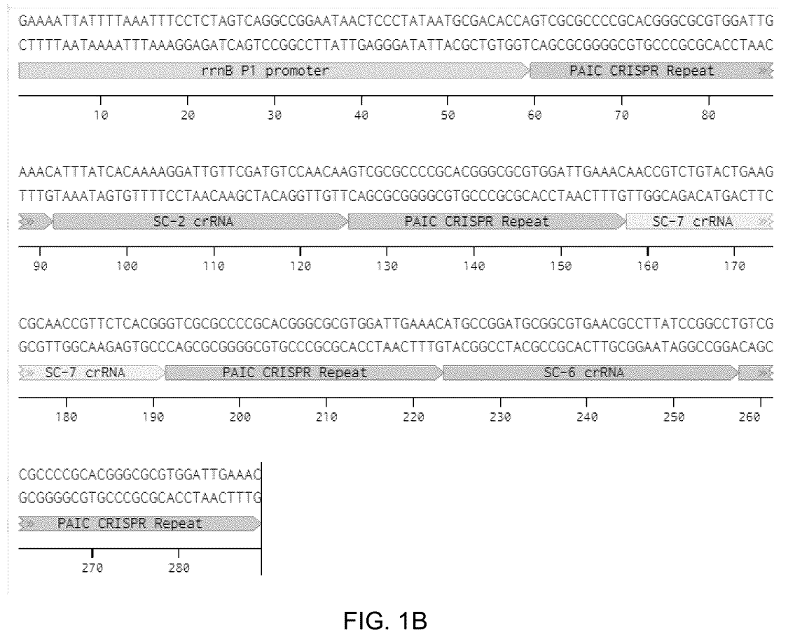

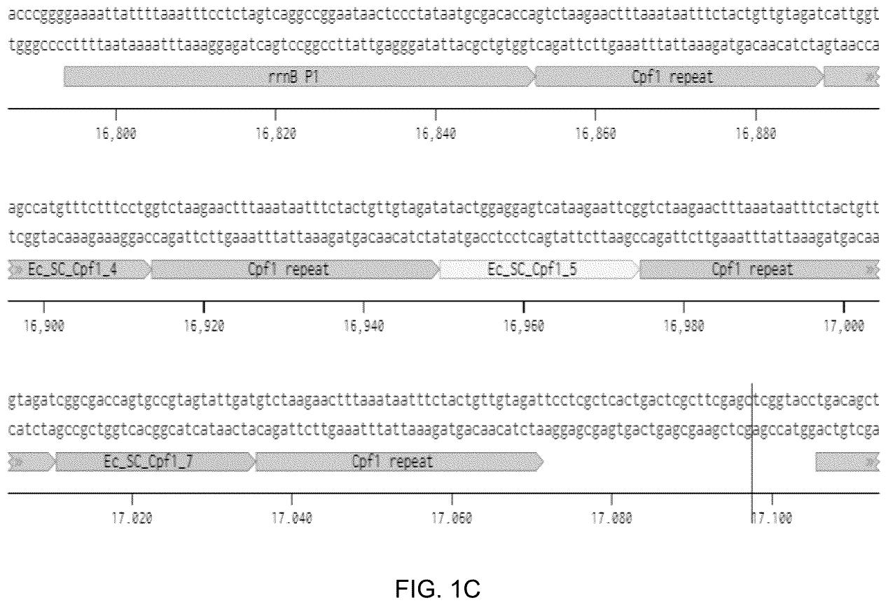

Certain bacteriophages were engineered to contain a crArray and Cas construct and/or encode a colicin. Table 1A depicts the components of the bacteriophages used in the following application. Table 2 depicts the sequences of the promoters used to drive expression of both the crArray and the Cas promoter. Table 3 depicts the sequence of the spacer sequence in the crArray used to target specific sites. Table 4 depicts the repeat sequences. Further, FIG. 1A depicts the sequence and alignment of the preliminary crArray5 (SC2) used in the following examples. FIG. 1B provides the full array sequence and alignment for final cocktail bacteriophages.

Cpf1 systems and sequences are provided herein. Exemplary pJC_Cpf1 array sequence and alignment is detailed in FIG. 1C. Cpf1 sequence elements are also listed in Tables 1-4 and 23.

TABLE 1A
Components of bacteriophage
WILDTYPE BACTERIOPHAGE

| | | Patent | |
|---|---|---|---|
| | | Deposit | |
| Strain name | Genus | Number* | Payload |
| p004k | Tequatrovirus | PTA-127149 | N/A (WT) |
| p00c0 | Mosigvirus | PTA-127143 | N/A (WT) |
| p00ex | Tequatrovirus | PTA-127145 | N/A (WT) |
| p00jc | Phapecoctavirus | PTA-127147 | N/A (WT) |
| p00ke | Unclassified | PTA-127148 | N/A (WT) |
| | Myovirus | | |
| p5516 | Vectrevirus | PTA-127151 | N/A (WT) |
| p6921 | Tequintavirus | PTA-127576 | N/A (WT) |
| p6977 | Tequatrovirus | PTA-127577 | N/A (WT) |
| p6984 | Tequatrovirus | PTA-127578 | N/A (WT) |

| | | | | | | |
|---|---|---|---|---|---|---|
| | | Patent | | | | |
| | | Deposit | crArray | crArray | Cas | Cas |
| Strain name | Genus | Number* | Promoter | ID | Promoter | ID |

CRISPR Bacteriophage

| | | | | | | |
|---|---|---|---|---|---|---|
| p004Ke007 | Tequatrovirus | N/A | rrnB P1 | (SC2) | Bba_J23109 | PaIC |
| | | | | (SEQ ID | | |
| | | | | NO: 42) | | |
| p004Ke009 | Tequatrovirus | PTA-127150 | rrnB P1 | PAIC | Bba_J23109 | PaIC |
| | | | | array 2 | | |
| | | | | (FIG. 1B) | | |
| p00c0e030 | Mosigvirus | PTA-127144 | rrnB P1 | PAIC | Bba_J23109 | PaIC |
| | | | | array 2 | | |
| | | | | (FIG. 1B) | | |
| p00c0e103 | Mosigvirus | N/A** | rrnB P1 | PAIC | Bba_J23109 | PaIC |
| | | | | array 2 | | |
| | | | | (FIG. 1B) | | |
| p00exe014 | Tequatrovirus | PTA-127146 | rrnB P1 | PAIC | Bba_J23109 | PaIC |
| | | | | array 2 | | |
| | | | | (FIG. 1B) | | |
| p00jce006 | Phapecoctavirus | N/A | rrnB P1 | Cpfl array | Bba_J23109 | Cpf1 |
| | | | | (SEQ ID | | |
| | | | | NO: 44) | | |
| p00exe003 | Tequatrovirus | N/A | Leader | Type I-E / | N/A | N/A |
| | | | sequence | I-F array | | |
| | | | | (SEQ ID | | |
| | | | | NO: 184) | | |
| p004ke005 | Tequatrovirus | N/A | Leader | Type I-E / | N/A | N/A |
| | | | sequence | I-F array | | |
| | | | | (SEQ ID | | |
| | | | | NO: 184) | | |
| p0031e002 | Tequatrovirus | N/A | Leader | Type I-E / | N/A | N/A |
| | | | sequence | I-F array | | |
| | | | | (SEQ ID | | |
| | | | | NO: 184) | | |

Colicin bacteriophage

| | | Patent | | |
|---|---|---|---|---|
| | | Deposit | | |
| Strain name | Genus | Number* | Promoter | Colicin |
| p00exe299 | Tequatrovirus | PTA-127579 | prom000106 | Colicin U (SEQ ID NO 168) |
| p00exe300 | Tequatrovirus | N/A | prom000106 | Colicin K (SEQ ID NO 167) |
| p00exe296 | Tequatrovirus | PTA-127582 | prom000012 | Colicin Ib (SEQ ID NO: 165) |
| p004ke127 | Tequatrovirus | PTA-127580 | prom000106 | Colicin K (SEQ ID NO 167) |
| p004ke124 | Tequatrovirus | N/A | prom000012 | Colicin Ib (SEQ ID NO: 165) |
| p00jce098 | Phapecoctavirus | PTA-127581 | prom000012 | Colicin 10 (SEQ ID NO 166) |
| p00jce172 | Phapecoctavirus | N/A | prom000106 + | Colicin K (SEQ ID NO 167) |
| | | | prom000073 | |
| p00kee072 | Unclassified | N/A | prom000012 | Colicin 10 (SEQ ID NO 166) |
| | Myovirus | | | |
| p00c0e105 | Mosigvirus | N/A | prom000106 | Colicin K (SEQ ID NO 167) |
| p00kee154 | Unclassified | N/A | prom000012 | Colicin Ib (SEQ ID NO: 165) |
| | Myovirus | | | |
| p00kee162 | Unclassified | N/A | prom000106 | Colicin U (SEQ ID NO 168) |
| | Myovirus | | | |
| p004ke125 | Tequatrovirus | N/A | prom000106 | Colicin K (SEQ ID NO 167) |
| p00jce096 | Phapecoctavirus | N/A | prom000012 | Colicin Ib (SEQ ID NO: 165) |
| p00exe297 | Tequatrovirus | N/A | prom000106 | Colicin K (SEQ ID NO 167) |
| p00exe291 | Tequatrovirus | N/A | prom000012 | Colicin 10 (SEQ ID NO 166) |

*Select bacteriophage were deposited with the ATCC Oct. 21, 2021 and Apr. 21, 2023.
**p00c0e030 comprises p00c0e030 (ATCC Deposit No ) with a deletion of SEQ ID NO: 183

TABLE 1B
Bacteriophage cocktails

| | | CK000618 | | | | LBP- |
|---|---|---|---|---|---|---|
| Phage | Genus | (LCP-ECO1 v.2) | CK000479 | CK000507 | CK000570 | EC01 v.1 |
| p00ex | Tequatrovirus | Full Construct | Wild Type | Half Construct | Full Construct | Half Construct |
| | | (p00exe014) | (p00ex) | (p00exe003) | (p00exe014) | (p00exe003) |
| p004k | Tequatrovirus | Full Construct | Wild Type | Half Construct | Full Construct | Half Construct |
| | | (p004ke009) | (p004k) | (p004ke005) | (p004ke009) | (p004ke005) |
| p00jc | Phapecoctavirus | Wild Type | Wild Type | Wild Type | Full Construct | N/A |
| | | (p00jc) | (p00jc) | (p00jc) | (p00jce006) | |
| p00ke | Unclassified | Wild Type | Wild Type | Wild Type | Wild Type | N/A |
| | Myovirus | (p00ke) | (p00ke) | (p00ke) | (p00ke) | |
| p00c0 | Mosigvirus | Full Construct | Wild Type | Wild Type | Full Construct | NA |
| | | (p00c0e103) | (p00c0) | (p00c0) | (p00c0e030) | |
| p5516 | Vectrevirus | Wild Type | Wild Type | Wild Type | Wild Type | N/A |
| | | (p5516) | (p5516) | (p5516) | (p5516) | |
| p0031 | Tequatrovirus | N/A | N/A | N/A | N/A | Half Construct |
| | | | | | | (p0031e002) |
| p0078 | Tequatrovirus | N/A | N/A | N/A | N/A | N/A |
| p00c8 | Phapecoctavirus | N/A | N/A | N/A | N/A | N/A |
| p0033L | Unclassified | N/A | N/A | N/A | N/A | N/A |
| | Myovirus | | | | | |

* Half construct refers to a bacteriophage containing a nucleic acid encoding a CRISPR array; Full construct refers to a bacteriophage containing a nucleic acid encoding a CRISPR array and a Cas3/CASCADE complex

TABLE 2
Promoter sequences

| SEQ | | | |
|---|---|---|---|
| ID | | | |
| NO | Promoter | Source | Sequence |
| 1 | ACR | phage | ACAAGCGGCACATTGTGCCTATTGCGAATTAGGCACAATGT |
| | | genome | GCCTAATCTAACGTCATGCCAGCCACAACGGCGAGGCGCC |
| | | | AAGAAGGATAGAAGCC |
| 2 | BBa_J23109 | BioBrick | TTTACAGCTAGCTCAGTCCTAGGGACTGTGCTAGC |
| 3 | endogenous | P. aeruginosa | GATTTTTTTCGGGTGAGGTTGCGGGCTGTTCGGTAGGTTTAT |
| | (promoter + | genome | AAACACTGCTATCCAAAGCTATGGACACGCTCGGCTACGA |
| | RBS) | | GAACAGTTGGCGTGATGGCCTCTAGCAATTAGATTGTTATG |
| | | | CGACATCCGCAGACTTGGCAGGGAGCGCACCT |
| 4 | BBa_J23106 | BioBrick | TTTACGGCTAGCTCAGTCCTAGGTATAGTGCTAGC |
| 5 | P16 | P. aeruginosa | ATCCGAGGGATACGGGCCTTGTCAGCACGGTGTTGCTAATG |
| | (promoter + | genome | AGAGCCTTTGCCCGGGCAATAGTACGGGCAGTGTGTAGCG |
| | RBS) | | GATTGAAAGACGCTGAATCACTGACAGGCATGAAGACTAT |
| | | | CGATAGAGTCTGATAGTGTCGCCGCCGCACAGCGGATAGA |
| | | | GTCCACAGTCATTGAAGTGTTAATCCGCGATCAAGCTC |
| 6 | gp105 | phage | GACCTAGCTTTTATAGCGGGTTTCGTGGTTTATAGCCCATTG |
| | (promoter + | genome | AAAAAAATCTCACATCTATATCACAGGTGTGCACTCGTTCC |
| | RBS) | | CGAAAGGTTCTGAGTCTACTTGATCAAGTATTGAAATACCA |
| | | | TCGTAAAGGAAAAAGACATGTCTATTCGTGATAGCGAAAA |
| | | | CAACAACGGCCAACAGCAGCAGACCGCGCAAACTGCCGCC |
| | | | CCCGCCCCGCAA |
| 7 | gp245 | phage | TTCAATTTAAGTAGTAACGAGGTCAGCCCGGAATCTTTGGG |
| | (promoter + | genome | TATTCTTAAGGTATTTCTGACTCAGTGTGGTTGGGACAGCTT |
| | RBS) | | CACTGTACATTGCACTGGATTTGTTAATTTCTTATACCGGGG |
| | | | CACCATGGGCAGCAAATCGTGTTACGAATTCCGTCTAACCA |
| | | | ATAAGCGAGCTAAATA |
| 8 | Pamp | plasmid | CGCGGAACCCCTATTTGTTTATTTTTCTAAATACATTCAAAT |
| | | | ATGTATCCGCTCATGAGACAATAACCCTGATAAATGCTTCA |
| | | | ATAATATTGAAAAAGGAAGAGT |
| 9 | Plpp | P. aeruginosa | CTTCAAGAATTCGTATTGACCCCATAGACAGCTTCGTCGAC |
| | (promoter + | genome | GCCCGTCCCGGCCCCCTTGGGCTTGCCGGACGGCTTATGTC |
| | RBS) | | ATGATGGCGCCACCCTCGCAGGTTCAAGGCCGGCTTTCTTC |
| | | | CTCTATGAACAAATCCCTTGCGCTGACTACGTAATCAC |
| 10 | Ptat | P. aeruginosa | CTTCAAGAATTCGGGGTATTCCTGATCCTGCGCCGCTAGCG |
| | (promoter + | genome | CCGCGCACGGCCACTAGGCCCGCGCCGATAGCCAGTCGCG |
| | RBS) | | CTCCCGGCTGGCACACTACTCCCATTTCCGCCGGAAACGCG |
| | | | CGCAACGTACCGGCAACGAACGTGGAAAGACCATGAAAGA |
| | | | CTGGCTGGATGAGATTCACTGGAACGCCGTGACCTACGTAT |
| | | | GCAC |
| 11 | rrnB P1 | E. coli | GAAAATTATTTTAAATTTCCTCTAGTCAGGCCGGAATAACT |
| | | | CCCTATAATGCGACACCA |
| 19 | BBa_J23109 | BioBrick | TTTACAGCTAGCTCAGTCCTAGGGACTGTGCTAGCATTAAA |
| | (promoter + | | GAGGAGAAAATG |
| | RBS) | | |
| 64 | BBa_J23102 | BioBrick | TTGACAGCTAGCTCAGTCCTAGGTACTGTGCTAGC |
| 65 | Leader | E. coli | GATAATTAGTGCTGCGGGTAGGTAAGGATAAAAAAGGGTG |
| | Sequence | | GCAGCAGGAGATTGAGATGGTTTTGCTTTATTAACAACGGG |
| | | | CTAAACGTGTAGTATTTGA |

TABLE 3
Spacer and array sequences

| | Spacer | SEQID | |
|---|---|---|---|
| Array ID | Target | NO | Sequence |
| SC2 | ppSa | 12 | ATTTATCACAAAAGGATTGTTCGATGTCCAACAA |
| SC1 | rpoD | 20 | CGGCAGATGGTCATTGACCTCGGCATAGGTCAGA |
| SC3 | cydB | 21 | ATAATCGATCATTTGACGACTCCTGTCTTAGCGT |
| SC4 | ihFa/pheT | 22 | TGACATTTCAGCTTTTGTAAGCGCCATAGGTTCA |
| SC5 | raiA | 23 | GCCTTTGTGCTGCAGTTTATTGAGCTGCCGTTCC |
| SC6 | intergenic | 24 | ATGCCGGATGCGGCGTGAACGCCTTATCCGGCCT |
| | conserved | | |
| | repeat | | |
| SC7 | rpmH | 25 | AACCGTCTGTACTGAAGCGCAACCGTTCTCACGG |
| SC8 | accB | 26 | GTAAGATTAAAAAACTGATCGAGCTGGTTGAAGA |
| SC9 | gyrB | 27 | TTATGACTCCTCCAGTATCAAAGTCCTGAAAGGG |
| SC10 | rplS | 28 | GTAACCGCGGTCTGCACTCTGCATTCACTGTTCG |
| SC11 | leuS | 29 | CCTATCCTTCTGGTCGACTACACATGGGCCACGT |
| SC12 | rpsD | 30 | TGATCTGTCTGCGGACATTAACGAACACCTGATC |
| SC13 | rplE | 31 | TGTCATGCAAGTCCCTCGGGTCGAGAAGATCACC |
| SC14 | rplR | 32 | CAATATCATGGTCGTGTCCAGGCACTGGCAGATG |
| SC15 | rpsU | 33 | TATGAAAAACCGACTACCGAACGTAAGCGCGCTA |
| SC16 | fusA | 34 | TGAACAACGAAATCATCCTGGTAACCTGTGGTTC |
| Cpf1_4 | UMP kinase | 35 | CATTGGTAGCCATGTTTCTTTCCTG |
| Cpf2_5 | DNA gyrase | 36 | ATACTGGAGGAGTCATAAGAATTCG |
| | subunit B | | |
| Cpf3_7 | 30s | 37 | CGGCGACCAGTGCCGTAGTATTGAT |
| | ribosomal | | |
| | protein | | |
| SC2 array | ppSa | 38 | GTCGCGCCCCGCACGGGCGCGTGGATTGAAACATTTATCACA |
| | | | AAAGGATTGTTCGATGTCCAACAAGTCGCGCCCCGCACGGGC |
| | | | GCGTGGATTGAAAC |
| PAIC | ppSa, | 39 | GTCGCGCCCCGCACGGGCGCGTGGATTGAAACATTTATCACA |
| array 2 (4 | intergenic | | AAAGGATTGTTCGATGTCCAACAAGTCGCGCCCCGCACGGGC |
| repeats) | conserved | | GCGTGGATTGAAACAACCGTCTGTACTGAAGCGCAACCGTTC |
| | repeat, and | | TCACGGGTCGCGCCCCGCACGGGCGCGTGGATTGAAACATGC |
| | rpmH | | CGGATGCGGCGTGAACGCCTTATCCGGCCTGTCGCGCCCCGC |
| | | | ACGGGCGCGTGGATTGAAAC |
| PAIC | ppSa, | 40 | ATTTATCACAAAAGGATTGTTCGATGTCCAACAAGTCGCGCC |
| array 2 (2 | intergenic | | CCGCACGGGCGCGTGGATTGAAACAACCGTCTGTACTGAAGC |
| repeats) | conserved | | GCAACCGTTCTCACGGGTCGCGCCCCGCACGGGCGCGTGGAT |
| | repeat, and | | TGAAACATGCCGGATGCGGCGTGAACGCCTTATCCGGCCT |
| | rpmH | | |
| Cpf1 | UMP kinase, | 41 | GTCTAAGAACTTTAAATAATTTCTACTGTTGTAGATCATTGGT |
| array | DNA gyrase | | AGCCATGTTTCTTTCCTGGTCTAAGAACTTTAAATAATTTCTA |
| | subunit B, | | CTGTTGTAGATATACTGGAGGAGTCATAAGAATTCGGTCTAA |
| | and 30s | | GAACTTTAAATAATTTCTACTGTTGTAGATCGGCGACCAGTGC |
| | ribosomal | | CGTAGTATTGATGTCTAAGAACTTTAAATAATTTCTACTGTTG |
| | protein | | TAGAT |
| SC2 array | ppSa | 42 | GAAAATTATTTTAAATTTCCTCTAGTCAGGCCGGAATAACTCC |
| with | | | CTATAATGCGACACCAGTCGCGCCCCGCACGGGCGCGTGGAT |
| example | | | TGAAACATTTATCACAAAAGGATTGTTCGATGTCCAACAAGT |
| promoter | | | CGCGCCCCGCACGGGCGCGTGGATTGAAAC |
| PAIC | ppSa, | 43 | GAAAATTATTTTAAATTTCCTCTAGTCAGGCCGGAATAACTCC |
| array 2 | intergenic | | CTATAATGCGACACCAGTCGCGCCCCGCACGGGCGCGTGGAT |
| with | conserved | | TGAAACATTTATCACAAAAGGATTGTTCGATGTCCAACAAGT |
| example | repeat, and | | CGCGCCCCGCACGGGCGCGTGGATTGAAACAACCGTCTGTAC |
| promoter | rpmH | | TGAAGCGCAACCGTTCTCACGGGTCGCGCCCCGCACGGGCGC |
| (FIG. 1B) | | | GTGGATTGAAACATGCCGGATGCGGCGTGAACGCCTTATCCG |
| | | | GCCTGTCGCGCCCCGCACGGGCGCGTGGATTGAAAC |
| Cpf1 | UMP kinase, | 44 | GAAAATTATTTTAAATTTCCTCTAGTCAGGCCGGAATAACTCC |
| array with | DNA gyrase | | CTATAATGCGACACCAGTCTAAGAACTTTAAATAATTTCTACT |
| example | subunit B, | | GTTGTAGATCATTGGTAGCCATGTTTCTTTCCTGGTCTAAGAA |
| promoter | and 30s | | CTTTAAATAATTTCTACTGTTGTAGATATACTGGAGGAGTCAT |
| | ribosomal | | AAGAATTCGGTCTAAGAACTTTAAATAATTTCTACTGTTGTAG |
| | protein | | ATCGGCGACCAGTGCCGTAGTATTGATGTCTAAGAACTTTAA |
| | | | ATAATTTCTACTGTTGTAGAT |
| Type I-E/ | ftsA | 184 | GAGTTCCCCGCGCCAGCGGGGATAAACCGCTGAAGTAGAAA |
| I-F array | | | AACGTGTTACAGCATCAGTCGAGTTCCCCGCGCCAGCGGGG |
| | | | ATAAACCGGGAGAGAGTGAGCGATCCTCCGTTAACATAGTT |
| | | | CACTGCCGTACAGGCAGCTTAGAAAGTATTATTCGACGGCG |
| | | | GTGGGATTGCTTCACGTTCACTGCCGTACAGGCAGCTTAGA |
| | | | AA |

TABLE 4
Repeat Sequences

| Repeat | SEQ ID NO: | Sequence |
|---|---|---|
| Repeat 1 | 13 | GTCGCGCCCCGCACGGGCGCGTGGATTGAAAC |
| Repeat 2 | 14 | GTCGCGCCCCGCACGGGCGCGTGGAGTGAAAG |
| Repeat 3 | 15 | GTCGCGCCCCGCACGGGTGCGTGGATTGAAAC |
| Repeat 4 | 16 | GTCGCGCCCCGCATGGGCGCGTGGATTGAACA |
| Repeat 5 | 17 | GTCGCGCCCTACGCGGGCGCGTGGAGTGAAAG |
| Cpf1 repeat | 18 | GTCTAAGAACTTTAAATAATTTCTACTGTTGTAGAT |
| Type I-E | | GAGTTCCCCGCGCCAGCGGGGATAAACCG |
| repeat | | |
| Type I-F | | GTTCACTGCCGTACAGGCAGCTTAGAAA |
| repeat | | |

### Example 2: Targeting crArrays in E. coli

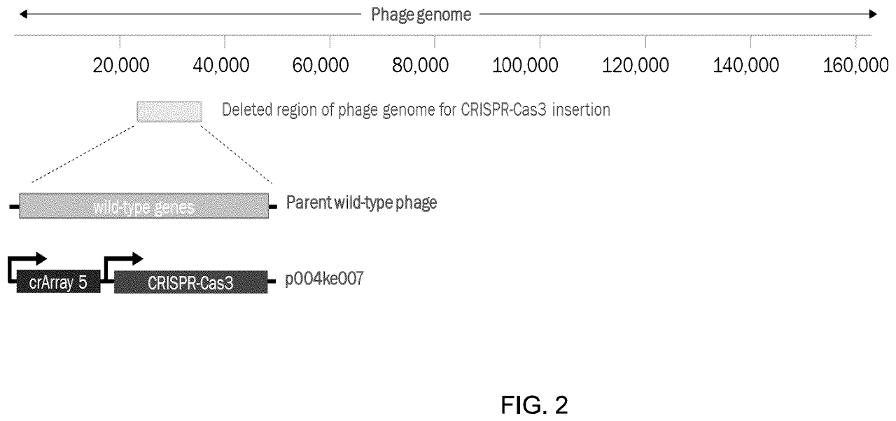

FIG. 2 shows a schematic representation of the genome of wild type bacteriophage p004k and its engineered variant p004ke007. The bar below the genome axis indicates the region of the genome that was removed and replaced. The schematic below the bacteriophage genome illustrates the DNA that was used to replace WT bacteriophage genes in the deleted region.

E. coli bacteriophage p004k (wild type) and p004ke007 (targeting SC2+Cas system) were mixed with the indicated bacterial strain while the bacteria was in logarithmic growth and plated onto LB agar 3 hours post inoculation. The ratio of bacteriophage to bacteria was altered by performing a dilution series of the bacteriophage, so that the amount of bacteria remained constant in each spot but the amount of bacteriophage changed. The relative ratio of the bacteriophage and bacteria shifted over the course of the experiment as the bacteria replicate and succumb to the bacteriophage, which is why an MOI is not listed. The label at the top of each set of images denotes the strain shown.

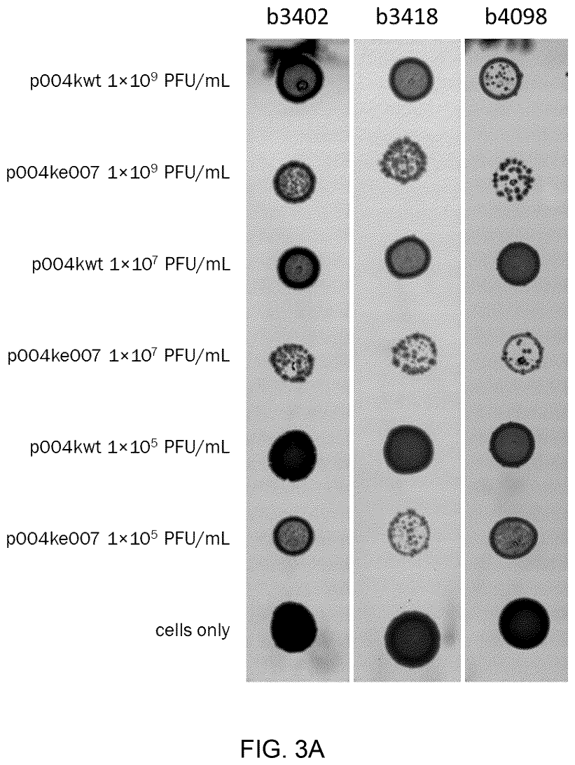

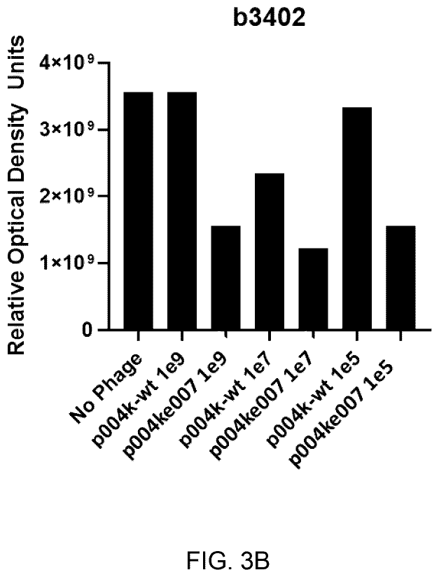

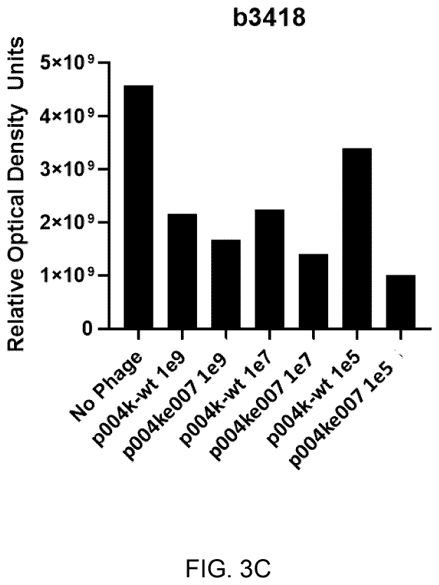

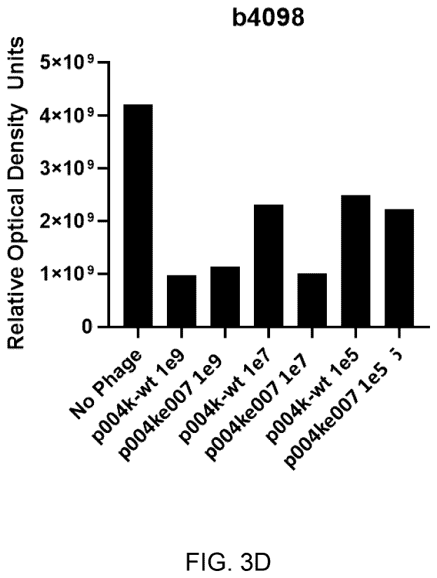

In this assay, the bacteriophage was mixed 1:1 with the indicated bacterial culture in mid-logarithmic growth to obtain the final bacteriophage titers listed on the left side of the image. The bacteria-phage mixture was incubated for 3 hours and then 2 ul of the culture was spotted onto LB plates, as depicted in FIG. 3A. Bacteria that survived the bacteriophage replicate to form visible colonies, so fewer bacteria means better bacteriophage kill. In this assay, the engineered bacteriophage appeared to kill the bacteria better than wildtype at all dilutions, but the 1×10⁷ dilution was the most visually striking for each E. coli strain. FIGS. 3B-3D show quantification of the images in FIG. 3A. The quantification was determined by comparing the relative optical density of each spot (essentially how dark each spot is, with a darker spot being indicative of more cell growth). Together, these data show that the engineered bacteriophage containing a Cas systems and crArray targeting the bacterial genome has improved kill over the wild type bacteriophage parent in multiple strains. These data demonstrate that bacteriophage expressing exogenous Cas systems improve the bacteriophage against diverse human pathogens.

### Example 3: Designing and Validating Spacer Sequences to Target a Target Bacterium

### Spacer Design

A spacer sequence is designed using the following protocol. First, suitable search set of representative genomes for the organism/species/target of interest are acquired. Examples of suitable databases include NCBI genbank and the PATRIC (Pathosystems Resource Integration Center) database. The genomes are downloaded in bulk via FTP (File Transfer Protocol) servers, enabling rapid and programmatic dataset acquisition.

The genomes are searched with relevant parameters to locate suitable spacer sequences. The genomes can be read from start to end, in both the forward and reverse complement orientations, to locate contiguous stretches of DNA that contain a PAM (Protospacer Adjacent Motif) site. The spacer sequence will be the N-length DNA sequence 3′ adjacent to the PAM site, where N is specific to the Cas system of interest and is generally known ahead of time. Characterizing the PAM sequence and spacer sequences are generally performed during the discovery and initial research of a Cas system. Every observed PAM-adjacent spacer can be saved to a file and/or database for downstream use.

Next, the quality of a spacer for use in a CRISPR engineered bacteriophage is determined using the following process. First, each observed spacer can be evaluated to determine how many of the evaluated genomes they are present in. The observed spacers can additionally be evaluated to see how many times they may occur in each given genome. Spacers that occur in more than one location per genome can be advantageous because the Cas system may not be able to recognize the target site if a mutation occurs, and each additional “backup” site increases the likelihood that a suitable, non-mutated target location will be present. The observed spacers can be evaluated to determine whether they occur in functionally annotated regions of the genome. If such information is available, the functional annotations can be further evaluated to determine whether those regions of the genome are “essential” for the survival and function of the organism. Focusing on spacers that occur in all, or nearly all, evaluated genomes of interest (>=99) ensures broad applicability to justify the spacer selection. Provided a large selection pool of conserved spacers exists, preference may be given to spacers that occur in regions of the genome that have known function, with higher preference given if those genomic regions are “essential” for survival and occur more than 1 time per genome.

### Spacer Validation

The identified spacer sequences can then be validated by completing the following procedure. First, a plasmid that replicates in the organism(s) of interest and has a selectable marker (e.g. an antibiotic-resistance gene) is identified. The genes encoding the Cas system are inserted into the plasmid such that they will be expressed in the organism of interest. Upstream of the Cas system, a promoter is included that is recognized by the organism of interest to drive expression of the Cas system. Between the promoter and the Cas system, a ribosomal binding site (RBS) is included that is recognized by the organism of interest.

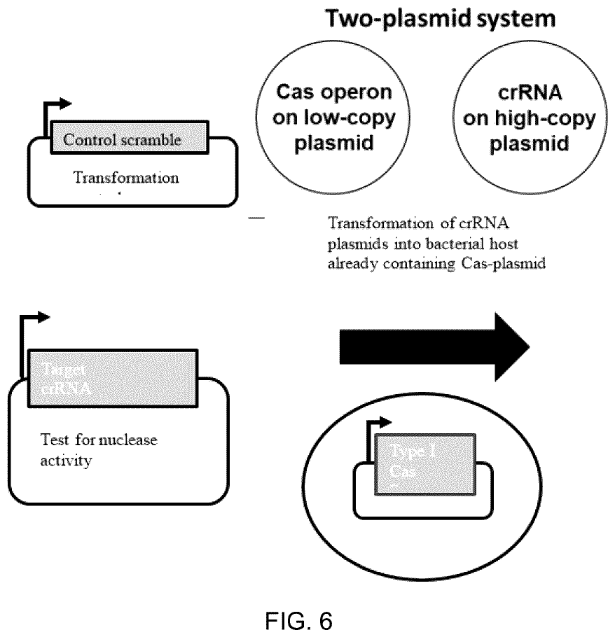

A two-plasmid system was used (illustrated in FIG. 6). One plasmid contained the CRISPR-Cas system expressed via a constitutive promoter (same promoter and RBS as in engineered bacteriophage) and a kanamycin resistance marker for selection. This plasmid was transformed into E. coli.

A second plasmid contained the individual crRNA expressed via a constitutive promoter (same promoter as in engineered bacteriophage) and an ampicillin resistance marker for selection. Electrocompetent cells were prepared from the E. coli strain harboring the CRISPR-Cas plasmid. The crRNA containing plasmid was then transformed into this strain.

Next, the killing efficacy of each tested spacer is determined. The plasmids listed in Table 5 are normalized to the same molar concentration. Each plasmid is transferred to the organism of interest by transformation, conjugation, or any other method for introducing a plasmid into a cell. The transformed cells are plated onto the appropriate selective media (e.g. antibiotic-containing agar). Following cell growth into colonies, the colonies that resulted from each different plasmid transfer are enumerated. Plasmids containing targeting spacers with a significantly lower transfer rate than the control plasmid containing the non-targeting spacer are considered to be successful at targeting the bacterial genome.

TABLE 5
Plasmids and controls used

| Plasmid | Function |
|---|---|
| Empty backbone vector | Control for plasmid transfer efficiency |
| Vector containing the cas system | Control for cas system toxicity |
| Vector containing the cas system | Control for off-target effects |
| and the non-targeting spacer | |
| Vector containing the cas system | Test sample |
| and the targeting spacer | |

### Example 4: Overview of E. coli Engineering Workflow

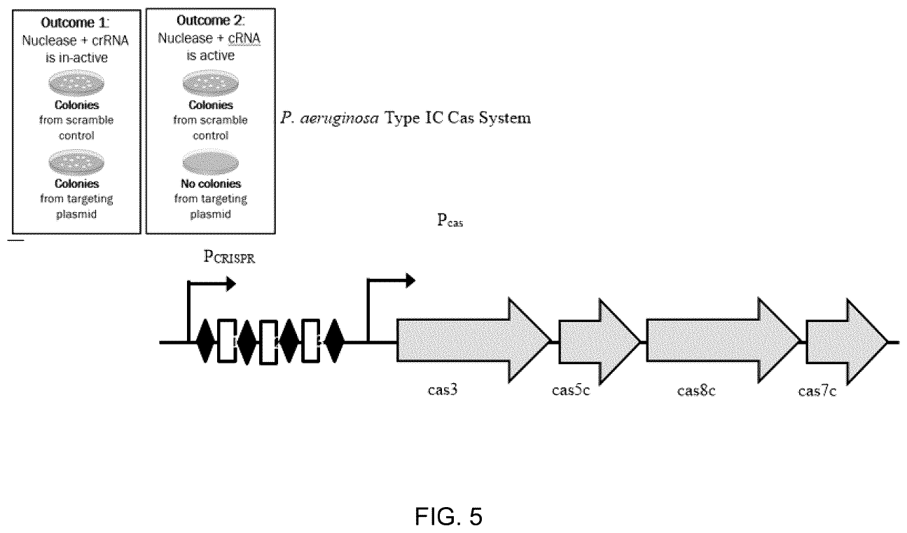

FIG. 5 shows an exemplary CRISPR-Cas system, the P. aeruginosa Type 1C Cas System. The PAIC gene operon is nearly 5700 bp, comprised of 4 separate proteins. Array of crRNA is expressed separately.

A plasmid-based assay was utilized to test efficacy of crRNA (illustrated in FIG. 6). A two-plasmid system was used. One plasmid contained the CRISPR-Cas system expressed via a constitutive promoter (same promoter and RBS as in engineered bacteriophage) and a kanamycin resistance marker for selection. This plasmid was transformed into E. coli.

A second plasmid contained the individual crRNA expressed via a constitutive promoter (same promoter as in engineered bacteriophage) and an ampicillin resistance marker for selection. Electrocompetent cells were prepared from the E. coli strain harboring the CRISPR-Cas plasmid. The crRNA containing plasmid was then transformed into this strain.

The number of colony forming units (CFU) formed after the transformation of either an active crRNA or a “scramble” control (randomized nucleotide sequence of same length as normal crRNA) were compared. The fewer colonies that developed, the greater the reduction attributable to the combination of the crRNA plasmid and the CRISPR-Cas plasmid.

For example, if the control crRNA plasmid transformation led to 1E7 CFUs and an active crRNA plasmid transformation led to 1E4 CFUs, that would represent a 3-log reduction attributed to CRISPR-Cas induced bacterial killing

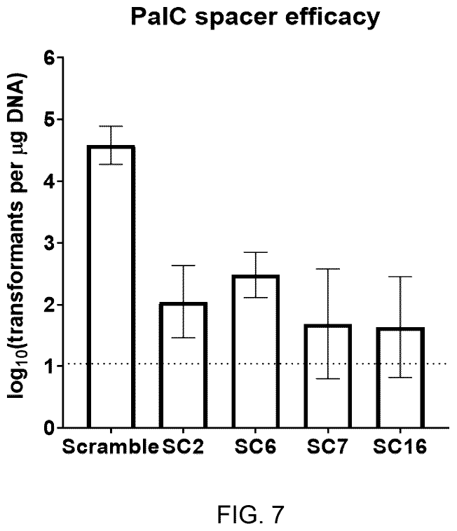

As shown in FIG. 7, the nominated spacers have high coverage and efficacy. The initial list of crRNA were chosen based on how conserved their target sequence was on a panel of ˜10,000 E. coli genomes publicly available. The efficacy column was determined based on the previously described plasmid-based assay. Highlighted spacer targets are the ones chosen for array testing. Selection was based on multiple parameters, including but not limited to percent coverage, efficacy and target among others.

### Example 5. Testing Efficacy of crRNA Array

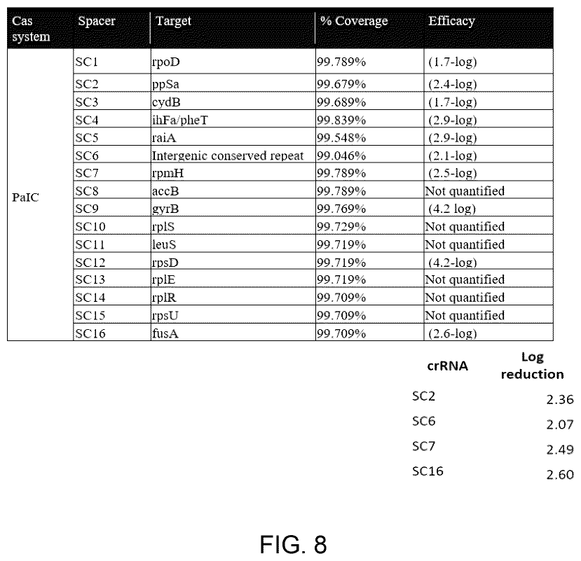

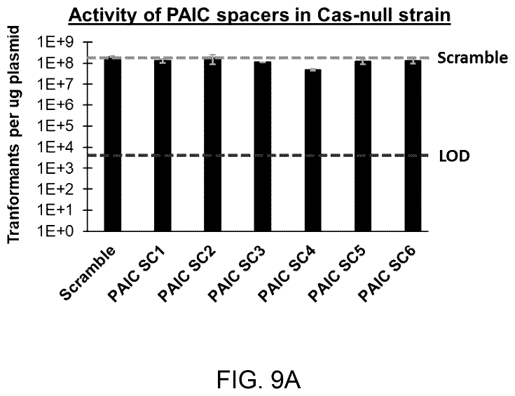

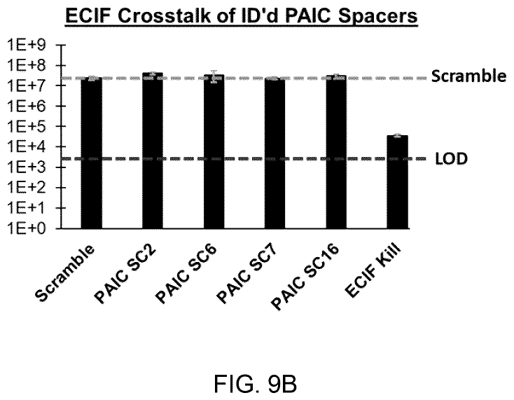

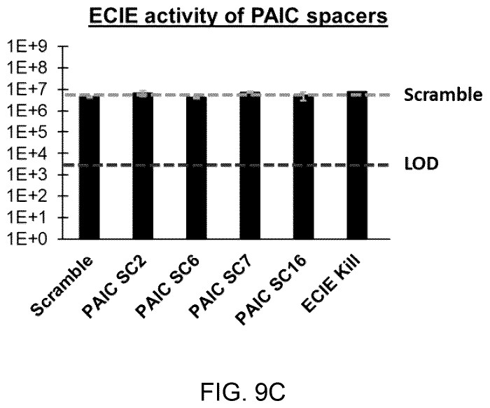

The spacer arrays were tested using the methods described above. FIG. 8 shows exemplary representative PaIC spacer efficiency, showing plasmid-based transformation results for 4 selected crRNA. Compared to scramble control, all four showed an approximately 2 log decreased in E. coli colonies. Replicates with no colonies are imputed to be ½% of limit of detection (LOD). Data was plotted as mean±S.D. The reduction in CFUs was not explained by general plasmid toxicity or another mechanism (i.e., antisense repression) as substantial CFU reductions are not observed in a Cas-null strain of E. coli (no PAIC CRISPR-Cas plasmid) (FIG. 9A). Additionally, there was no measurable reduction in CFU formation when the selected crRNA plasmids are transformed into E. coli strains possessing native CRISPR-Cas systems. A crRNA designed for the E. coli type IF (EcIF) system led to a multiple log reduction in CFUs, which indicate high specificity of the crRNA design, and absence of cross-talk of the identified PaIC spacers (FIG. 9B). A crRNA designed for the E. coli type IE (EcIE) did not activate the endogenous Cas system due to the absence of LeuO transcriptional regulator required to activate the native EcIE systems (FIG. 9C). K12 E. coli strain DH5-alpha strain was used for EcIE testing and E. coli NC101 strain was used for EcIF testing in this assay.

### Example 6. A Prediction Model for Number of Sites to be Targeted that can Sufficiently Avoid Mutational Escape

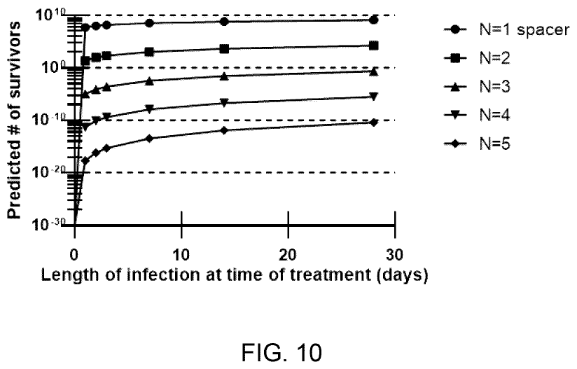

A basic model of mutation frequency was developed to understand the number of independent CRISPR-Cas targets needed to prevent escape. Data shown in FIG. 10 suggests that targeting 3 independent sites with CRISPR-Cas is sufficient to mitigate risk of mutational escape. This model assumes several worst-case parameters for example: (a) Normal mutation rate of 1e-6 per genome; (b) High bioburden of 1014 CFU (total GI microbiome or complete coating of ˜100 m2 lung surface area); (c) High growth rate in vivo (20 minute doubling time); (d) Any mutation completely eliminates CRISPR-Cas activity. Under these highly stringent assumptions, 3 spacers are sufficient to completely suppress the emergence of resistance for a month of continuous infection, or ˜2,000 bacterial generations at 1014 CFU.

### Example 7. Analysis of PaIC Arrays 1 and 2

Based on the assays described in the previous sections, at least two arrays were further analyzed, PaIC array 1 and PaIC array 2. Table 9 characterizes the arrays.

TABLE 9
PaIC array 1 and PaIC array 2

| | PAIC ARRAY 1 | | PAIC ARRAY 2 | |
|---|---|---|---|---|
| | SPACER | | SPACER | |
| | ID | COVERAGE | ID | COVERAGE |
| SPACER 1 | SC7 | 99.789% | SC7 | 99.789% |
| SPACER 2 | SC16 | 99.709% | SC6 | 99.046% |
| SPACER 3 | SC2 | 99.679% | SC2 | 99.679% |

| | | |
|---|---|---|
| TOTAL | 99.96% | 99.99% |

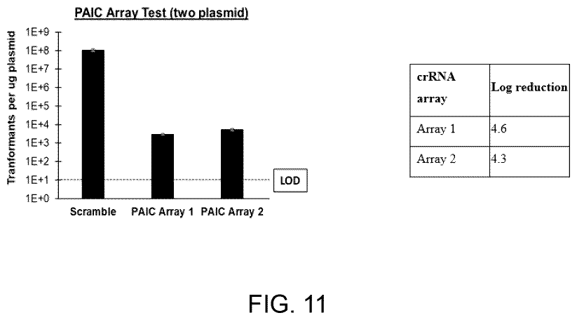

As indicated in Table 9, both the arrays have high overall coverage on about 10,000 clinical EC strains. A comparison of the array kill data using the plasmid based kill method described earlier, both PaIC arrays showed high competence in bacterial killing (FIG. 11). The array sequences are provided in Table 10 below.

TABLE 10
Nucleotide sequence of PaIC array 1 and PaIC array 2. Sequence includes
promoter, CRISPR repeats and three crRNA spacer sequences.

| Array | SEQ ID | Sequence |
|---|---|---|
| PAIC | 45 | gaaaattattttaaatttcctctagtcaggccggaataactccctataat |
| array 1 | | gcgacaccagtcgcgccccgcacgggcgcgtggattgaaacATTTATCA |
| | | CAAAAGGATTGTTCGATGTCCAACAAgtcgcgccccgcacgggcgcgtg |
| | | gattgaaacaaccgtctgtactgaagcgcaaccgttctcacgggtcgcg |
| | | ccccgcacgggcgcgtggattgaaactgaacaacgaaatcatcctggta |
| | | acctgtggttcgtcgcgccccgcacgggcgcgtggattgaaac |
| PAIC | 43 | Gaaaattattttaaatttcctctagtcaggccggaataactccctataat |
| array 2 | | gcgacaccagtcgcgccccgcacgggcgcgtggattgaaacATTTATCA |
| | | CAAAAGGATTGTTCGATGTCCAACAAgtcgcgccccgcacgggcgcgtg |
| | | gattgaaacaaccgtctgtactgaagcgcaaccgttctcacgggtcgcg |
| | | ccccgcacgggcgcgtggattgaaacATGCCGGATGCGGCGTGAACGCC |
| | | TTATCCGGCCTgtcgcgccccgcacgggcgcgtggattgaaac |

### Example 8: Purified and Cocktail E. coli Bacteriophage Host Range Assay

Data for purified and cocktail E. coli bacteriophage was acquired, reported in the best result from combined liquid and plaquing host range assays. The data summarized was the median of the binary hits across both liquid and plaquing host range for a given bacteriophage plus strain combination.

Determining liquid host range involved the addition of 5 μL of frozen, OD-controlled, culture material, 5 μL of known titer bacteriophage material and 40 μL of growth media into a well of a 364-well plate along with appropriate culture, bacteriophage, and media only controls. The plate was incubated for 20 hours at 37 C while shaking and OD600 readings were taken by the liquid handler every hour. The results were calculated by determining the ratio between areas under the cover (AUC) for samples with bacteriophage added and their respective controls. Samples with AUC ratios below 0.65 were considered positive (+) hits while AUC ratios greater than or equal to 0.65 were negative (−) hits.

For plaquing host range assays, bacterial strains of interest were cultured and screened for prophage. Bacteriophage of interest were serially diluted 50-fold across a microtiter plate from undiluted to 50-3 in 1×PBS. Agar overlays of strains used as titer host were poured and allowed to sit overnight. The following day, lysates for the bacterial strains of interest were spotted. After 15-20 min, the plates were imaged using the Hamilton-STAR-C062 and either counted by hand or run through an internally developed image analysis pipeline for transformation, background subtraction, and counting. Samples with a positive (+) number of plaque forming units were considered hits.

The results of this assay involving E. coli, wildtype Tequatrovirus bacteriophage (p004k and p00ex), wildtype Mosigvirus bacteriophage (p00c0), wildtype Phapecoctavirus bacteriophage (p00jc), wildtype Unique Myoviridae bacteriophage (p00ke), wildtype Vectrevirus bacteriophage (p5516), and cocktail CK507 were listed in Table 11. Cocktail CK507 comprises p004ke009, p00c0e030, p00ex014, p00jc, p00ke, and p5516. Overall, host range data was increased for cocktail CK507 as compared to purified bacteriophage.

TABLE 11
E. coli bacteriophage Host Range for Wild-Type
bacteriophage and Cocktail CK507

| | Individual bacteriophage | | | | | | Cocktail |
|---|---|---|---|---|---|---|---|
| Target | p004k | p00c0 | p00ex | p00jc | p00ke | p5516 | CK507 |
| b001024 | + | + | − | + | + | − | + |
| b001963 | − | − | − | − | − | − | + |
| b001965 | − | − | − | − | − | − | + |
| b001966 | − | − | + | + | + | − | + |
| b001991 | + | − | + | − | − | + | + |
| b001995 | − | − | − | − | − | − | + |
| b001997 | − | − | − | − | − | + | + |
| b001999 | − | − | + | + | + | − | + |
| b002027 | − | − | + | − | − | − | + |
| b002029 | − | − | − | − | − | + | + |
| b002030 | − | − | − | − | − | − | + |
| b002033 | + | − | − | − | − | − | + |
| b002034 | − | − | − | − | + | − | + |
| b002039 | − | − | + | − | − | − | + |
| b002073 | − | − | + | − | − | + | + |
| b002078 | − | − | − | − | − | − | + |
| b002105 | − | − | + | − | + | + | + |
| b002194 | + | − | + | − | − | + | + |
| b002205 | − | − | − | + | + | − | + |
| b002207 | − | − | + | − | − | − | + |
| b002208 | + | − | − | − | − | − | + |
| b002209 | + | − | − | − | − | − | + |
| b002232 | − | − | + | + | + | − | + |
| b002239 | − | − | − | + | + | + | + |
| b002245 | − | − | − | − | − | + | + |
| b002252 | − | − | − | + | + | − | + |
| b002257 | − | − | − | − | − | + | + |
| b002263 | − | − | − | + | − | − | + |
| b002301 | − | − | − | − | + | − | + |
| b002305 | − | − | − | + | − | − | + |
| b002308 | − | − | + | + | + | − | + |
| b002502 | − | − | + | − | − | + | + |
| b002503 | + | − | + | − | − | − | + |
| b002504 | − | − | + | + | − | − | + |
| b002505 | − | + | + | − | − | − | + |
| b002506 | − | − | + | − | − | − | + |
| b002507 | − | − | − | − | − | − | − |
| b002508 | − | − | − | − | − | − | + |
| b002509 | − | − | + | − | − | − | + |
| b002510 | − | − | − | − | − | − | + |
| b002511 | + | − | + | + | − | − | + |
| b002512 | − | − | + | − | − | − | + |
| b002513 | + | − | + | − | − | − | + |
| b002514 | − | − | − | − | − | − | − |
| b002515 | − | − | + | + | + | − | + |
| b002516 | + | − | + | − | − | − | + |
| b002517 | − | − | + | − | − | − | + |
| b002518 | − | − | + | − | − | + | + |
| b002519 | − | − | + | − | − | + | + |
| b002520 | + | − | − | − | − | − | + |
| b002521 | − | − | − | + | + | − | + |
| b002522 | + | − | + | − | − | − | + |
| b003247 | − | − | + | − | + | − | + |
| b003347 | − | − | − | − | − | − | + |
| b003348 | − | − | + | + | + | − | + |
| b003349 | − | + | + | + | + | − | + |
| b003350 | − | − | − | − | − | − | − |
| b003351 | − | − | − | − | − | − | − |
| b003352 | − | − | − | − | − | − | − |
| b003353 | − | − | + | − | − | − | + |
| b003354 | − | − | − | + | + | − | + |
| b003355 | − | − | + | + | − | − | + |
| b003356 | − | − | − | + | − | − | + |
| b003357 | − | − | + | + | + | − | + |
| b003358 | − | − | − | − | − | − | + |
| b003359 | − | − | − | − | − | − | − |
| b003360 | − | − | − | − | − | − | + |
| b003361 | + | − | + | + | − | − | + |
| b003362 | − | − | + | + | − | − | + |
| b003363 | + | − | − | − | − | − | + |
| b003364 | − | − | + | − | + | − | + |
| b003365 | − | − | − | − | − | − | − |
| b003366 | − | − | + | − | + | − | + |
| b003367 | − | − | − | − | − | − | − |
| b003368 | − | − | + | − | − | + | + |
| b003369 | + | − | + | + | − | − | + |
| b003370 | − | − | + | + | + | − | + |
| b003371 | − | − | − | + | + | − | + |
| b003372 | − | − | − | − | − | − | − |
| b003373 | − | − | − | − | − | − | − |
| b003374 | + | − | + | − | + | − | + |
| b003375 | − | − | + | − | − | + | + |
| b003376 | − | − | + | − | − | + | + |
| b003377 | − | − | + | − | − | − | + |
| b003378 | − | − | − | + | − | − | + |
| b003379 | − | − | + | − | − | + | + |
| b003380 | − | − | − | − | − | − | − |
| b003381 | + | − | + | − | − | − | + |
| b003382 | − | − | + | − | − | − | + |
| b003383 | − | − | − | + | + | − | + |
| b003384 | + | − | + | + | − | − | + |
| b003385 | − | − | + | − | − | + | + |
| b003386 | − | − | − | + | + | + | + |
| b003387 | + | − | + | + | + | − | + |
| b003388 | + | − | + | − | − | − | + |
| b003389 | − | − | + | − | − | − | + |
| b003390 | − | − | + | − | − | + | + |
| b003391 | + | − | + | + | − | − | + |
| b003392 | − | − | + | − | − | − | + |
| b003393 | − | − | − | − | − | − | + |
| b003394 | + | − | + | − | − | + | + |
| b003395 | + | − | + | + | + | − | + |
| b003396 | + | − | + | − | − | − | + |
| b003397 | + | − | + | − | − | − | + |
| b003398 | − | − | − | − | + | − | + |
| b003399 | − | − | − | − | − | − | + |
| b003400 | + | − | + | − | − | − | + |
| b003401 | + | − | + | − | − | − | + |
| b003402 | + | − | + | − | − | − | + |
| b003403 | − | − | − | + | + | − | + |
| b003404 | − | − | + | − | − | − | + |
| b003405 | − | − | + | + | + | − | + |
| b003406 | − | + | + | − | − | − | + |
| b003407 | + | − | + | − | − | + | + |
| b003408 | − | − | + | − | − | + | + |
| b003409 | + | − | + | + | + | − | + |
| b003410 | − | − | + | − | − | − | + |
| b003411 | + | − | + | + | + | − | + |
| b003412 | + | − | + | − | − | − | + |
| b003413 | − | − | − | − | − | + | + |
| b003414 | − | − | + | + | + | + | + |
| b003415 | − | − | − | − | − | − | + |
| b003416 | − | − | − | − | − | − | + |
| b003417 | + | − | + | + | + | + | + |
| b003418 | + | − | + | − | − | + | + |
| b003419 | + | − | − | − | − | − | + |
| b003420 | − | − | + | + | + | + | + |
| b003421 | − | − | − | − | − | − | + |
| b003422 | + | + | − | + | + | − | + |
| b003675 | − | − | + | − | − | + | + |
| b003762 | − | − | + | − | − | + | + |
| b003763 | − | − | − | − | − | − | + |
| b003764 | − | − | − | − | − | − | + |
| b003765 | + | − | − | − | − | − | + |
| b003770 | + | − | + | + | + | − | + |
| b003771 | − | − | − | + | − | − | + |
| b003773 | − | − | + | − | − | + | + |
| b003774 | + | − | + | − | − | − | + |
| b003777 | − | − | − | − | + | − | + |
| b003779 | − | − | − | − | − | − | + |
| b003780 | − | − | − | − | + | − | + |
| b003781 | − | − | − | − | − | − | − |
| b003782 | + | − | + | + | + | − | + |
| b003783 | − | − | + | + | + | − | + |
| b003786 | − | − | − | − | − | − | + |
| b003787 | − | − | + | − | − | − | + |
| b003788 | − | − | + | − | − | + | + |
| b003790 | − | − | + | − | − | − | + |
| b003791 | − | − | − | + | + | − | + |
| b003793 | − | − | + | − | − | + | + |
| b003795 | − | − | − | − | − | − | + |
| b003798 | − | − | + | − | − | + | + |
| b003802 | − | − | − | − | − | − | − |
| b003804 | − | − | − | − | − | − | + |
| b003805 | − | − | + | + | − | − | + |
| b003806 | − | − | − | − | − | − | + |
| b003812 | + | − | + | − | − | − | + |
| b003813 | + | − | + | − | + | − | + |
| b003815 | − | − | + | − | + | − | + |
| b003818 | + | − | − | + | + | − | + |
| b003821 | − | − | + | + | + | − | + |
| b003823 | + | − | + | + | + | − | + |
| b003826 | − | − | − | − | − | − | − |
| b003827 | − | − | − | − | − | − | + |
| b003829 | − | − | − | − | − | − | − |
| b003830 | − | − | + | − | − | + | + |
| b003838 | − | − | − | + | − | − | + |
| b003840 | − | − | − | − | − | − | − |
| b003841 | − | − | + | − | − | − | + |
| b003843 | − | − | − | − | − | − | − |
| b003845 | + | − | + | + | + | − | + |
| b003850 | − | − | − | − | − | − | − |
| b003851 | − | − | + | − | + | + | + |
| b003852 | + | − | + | + | + | − | + |
| b003853 | + | − | + | − | − | + | + |
| b003855 | + | − | + | − | − | − | + |
| b003856 | − | − | + | − | − | + | + |
| b003864 | + | − | + | − | − | − | + |
| b003865 | + | + | + | + | − | − | + |
| b003868 | + | − | − | + | + | − | + |
| b003871 | − | − | + | + | + | − | + |
| b003872 | − | − | + | − | − | − | + |
| b003874 | − | − | − | − | − | − | − |
| b003880 | − | − | − | + | − | − | + |
| b003885 | − | − | − | − | − | − | + |
| b003886 | − | − | − | − | − | − | + |
| b003887 | − | − | − | + | + | − | + |
| b003889 | + | − | + | + | − | − | + |
| b003890 | + | − | + | − | − | − | + |
| b003893 | − | − | − | + | + | − | + |
| b003894 | − | − | − | − | − | + | + |
| b003896 | − | − | − | − | − | − | + |
| b003897 | − | − | − | − | − | − | − |
| b003898 | − | − | + | − | − | + | + |
| b003899 | − | − | + | − | − | + | + |
| b003900 | − | − | + | − | − | − | + |
| b003902 | + | − | + | + | + | − | + |
| b003903 | − | − | + | − | − | + | + |
| b003907 | − | − | − | − | + | + | + |
| b003908 | − | − | − | + | + | + | + |
| b003909 | − | − | − | + | + | − | + |
| b003912 | − | − | + | + | + | + | + |
| b003920 | + | − | + | − | + | − | + |
| b003922 | − | − | − | − | − | + | + |
| b003923 | − | − | + | − | − | − | + |
| b003924 | − | − | + | − | − | + | + |
| b003926 | + | − | + | + | + | − | + |
| b003930 | − | − | + | − | − | + | + |
| b003931 | − | − | + | − | − | + | + |
| b003932 | − | − | + | − | + | + | + |
| b003934 | + | − | + | − | + | − | + |
| b003935 | − | − | − | − | − | − | − |
| b003941 | − | − | − | − | − | + | + |
| b003942 | − | − | − | − | − | − | + |
| b003943 | − | − | − | − | − | − | − |
| b003944 | − | − | − | − | + | − | + |
| b003945 | − | − | + | − | − | − | + |
| b003947 | − | − | − | − | − | − | + |
| b003948 | − | − | − | − | − | − | − |
| b003949 | − | − | − | + | − | − | + |
| b003952 | − | − | + | − | + | − | + |
| b003954 | − | − | − | − | − | − | − |
| b003956 | − | − | − | + | + | − | + |
| b003957 | − | − | + | + | + | − | + |
| b003958 | − | − | − | − | − | − | + |
| b003962 | + | − | + | − | − | − | + |
| b003963 | + | − | + | − | − | − | + |
| b003965 | − | − | − | − | − | − | − |
| b003969 | + | − | + | − | − | − | + |
| b003970 | − | − | + | − | − | + | + |
| b003974 | − | − | + | − | − | − | + |
| b003976 | − | − | − | − | − | − | + |
| b003977 | − | − | + | − | − | − | + |
| b003978 | + | − | − | + | + | − | + |
| b003979 | + | − | + | + | + | − | + |
| b003982 | + | − | + | − | − | − | + |
| b003983 | + | − | − | − | − | − | + |
| b003984 | − | − | + | + | + | − | + |
| b003986 | − | − | + | + | + | − | + |
| b003989 | − | − | − | + | − | − | + |
| b003990 | − | − | − | − | + | − | + |
| b003996 | − | − | − | − | − | − | − |
| b003998 | + | − | + | + | + | + | + |
| b004000 | − | − | + | − | − | + | + |
| b004008 | − | − | − | − | − | − | − |
| b004009 | − | − | + | + | − | − | + |
| b004012 | − | − | + | − | − | − | + |
| b004013 | − | − | − | − | − | − | + |
| b004014 | + | − | + | − | − | − | + |
| b004015 | − | − | + | − | − | + | + |
| b004016 | − | − | + | − | − | + | + |
| b004017 | − | − | − | + | + | − | + |
| b004018 | + | − | + | − | − | − | + |
| b004020 | − | − | − | + | − | − | + |
| b004021 | − | − | + | − | − | − | + |
| b004022 | − | − | − | − | − | − | − |
| b004023 | − | − | + | − | − | − | + |
| b004025 | − | − | − | − | − | − | − |
| b004026 | − | − | − | − | + | − | + |
| b004027 | − | − | + | + | + | − | + |
| b004028 | − | − | + | − | − | + | + |
| b004031 | + | − | + | − | − | − | + |
| b004032 | − | − | − | − | − | − | − |
| b004033 | − | − | − | − | − | − | + |
| b004034 | − | − | − | − | + | − | + |
| b004037 | − | − | − | + | − | − | + |
| b004038 | − | − | − | − | − | − | − |
| b004039 | − | − | − | − | − | − | + |
| b004040 | − | − | − | + | + | − | + |
| b004041 | − | − | − | − | − | − | + |
| b004043 | − | − | − | + | − | − | − |
| b004044 | − | − | − | + | + | − | + |
| b004045 | − | − | + | − | − | − | + |
| b004046 | + | + | + | + | + | − | + |
| b004048 | + | − | + | − | − | + | + |
| b004049 | − | − | − | − | − | − | + |
| b004052 | − | − | − | + | + | − | + |
| b004057 | − | − | + | − | − | − | + |
| b004058 | − | − | + | + | + | − | + |
| b004059 | − | − | − | + | + | − | + |
| b004060 | − | − | + | − | + | + | + |
| b004092 | − | − | + | − | − | + | + |
| b004096 | + | − | − | − | − | − | + |
| b004097 | − | − | − | − | − | − | − |
| b004098 | + | − | + | − | − | − | + |
| b004099 | − | − | − | − | + | − | + |
| b004100 | − | − | − | − | − | − | + |
| b004101 | − | − | + | − | − | − | + |
| b004102 | − | − | − | − | − | − | − |
| b004103 | − | − | − | − | − | − | − |
| b004104 | − | − | − | − | − | − | − |
| b004105 | − | − | + | − | − | + | + |
| b004106 | − | − | + | − | − | − | + |
| b004107 | − | − | − | − | − | − | − |
| b004108 | − | − | − | − | − | − | − |
| b004109 | − | − | − | − | − | − | − |
| b004110 | − | − | − | − | − | − | + |
| b004111 | − | − | − | − | − | − | + |
| b004112 | − | − | − | + | − | − | + |
| b004113 | − | − | − | − | − | − | − |
| b004114 | − | − | + | + | + | − | + |
| b004115 | − | − | − | + | + | − | + |
| b004116 | − | − | − | − | − | − | − |
| b004117 | − | − | + | − | − | + | + |

### Example 9: Full Construct in a Second E. coli Bacteriophage Assay

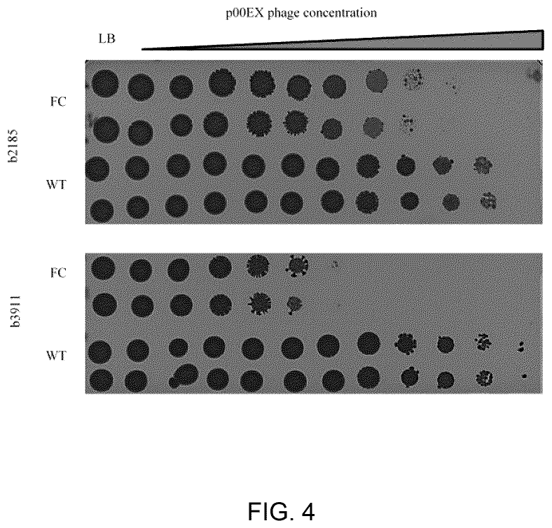

A standard plate kill assay starting at 3e10 PFU/ml (MOI of ˜100) and serial 1:5 dilutions of bacteriophage with the same bacterial concentration was completed after the E. coli bacteria (strains b2185 and b3911) and bacteriophage were mixed and immediately spotted onto a plate. p00Ex wildtype and engineered bacteriophage were mixed with bacteria in logarithmic growth and plated immediately in 2 ul spots on LB agar. The ratio of bacteriophage to bacteria was altered through the dilution series so that the amount of bacteria stayed constant at each dilution but the amount of bacteriophage was a 1 to 5 dilution. At the highest dilution, the multiplicity of infection (MOI) was 100, meaning there were approximately 100 bacteriophages per bacteria. In FIG. 4, p00EXe014 (FC) killed both the b2185 and b3911 strains consistently better than the p00EX wildtype (WT) counterpart. The data for both E. coli strains, validated the full construct bacteriophage was working as expected, validating the standard plate kill assay.

### Example 10. Purified Bacteriophage Cocktail CK618

A purified bacteriophage cocktail including six bacteriophages (p004k, p00jc, p00c0, p00ke, p5516 and p00ex) are described. Both potency and purity are evaluated for consistency with clinical trial material including endotoxin levels.

TABLE 12
Cfco

| | | CONSTRUCT |
|---|---|---|
| PHAGE | GENUS | INCORPORATED |
| P00EXE014 | Tequatrovirus | Full construct |
| P004KE009 | Tequatrovirus | Full construct |
| P00JC | Phapecoctavirus | Wild type |
| P00KE | Unclassified myovirus | Wild type |
| P00C0E030 | Mosigvirus | Full construct |
| P5516 | Vectrevirus | Wild type |

Table 13 shows E. coli bacteriophage Host Range in Cocktail CK618

| | Individual bacteriophage | | | | | | Cocktail |
|---|---|---|---|---|---|---|---|
| Target | p004ke009 | p00c0e030 | p00exe014 | p00jc | p00ke | p5516 | CK618 |
| b001024 | + | + | + | + | + | + | + |
| b001963 | − | − | + | − | + | + | + |
| b001965 | + | + | + | − | − | − | + |
| b001966 | + | + | + | + | + | + | + |
| b001991 | + | − | + | − | + | + | + |
| b001995 | − | + | + | + | − | − | + |
| b001997 | + | + | + | − | + | + | + |
| b001999 | + | + | + | + | − | + | + |
| b002027 | + | + | + | + | − | − | + |
| b002029 | − | + | + | − | + | + | + |
| b002030 | − | + | + | + | + | − | + |
| b002033 | + | + | + | + | − | − | + |
| b002034 | + | + | + | + | + | + | + |
| b002039 | + | + | + | − | + | + | + |
| b002073 | + | − | + | − | + | + | + |
| b002078 | − | + | + | − | − | − | + |
| b002105 | − | + | + | − | + | + | + |
| b002194 | + | − | + | − | + | + | + |
| b002205 | + | + | + | + | + | + | + |
| b002207 | + | + | + | + | + | + | + |
| b002208 | + | − | + | − | − | + | + |
| b002209 | + | + | + | − | + | + | + |
| b002232 | − | + | + | + | − | − | + |
| b002239 | − | − | + | + | + | + | + |
| b002245 | − | + | + | − | + | + | + |
| b002252 | − | + | + | + | + | − | + |
| b002257 | − | + | + | − | + | + | + |
| b002263 | − | + | + | + | + | + | + |
| b002301 | + | + | + | + | + | + | + |
| b002305 | − | + | + | + | − | − | + |
| b002308 | + | + | + | + | − | − | + |
| b002502 | + | + | + | − | + | + | + |
| b002503 | + | + | + | − | + | + | + |
| b002504 | − | + | + | + | − | − | + |
| b002505 | − | + | + | − | − | − | + |
| b002506 | − | + | + | + | − | − | + |
| b002507 | − | − | − | + | + | + | + |
| b002508 | + | + | + | + | + | + | + |
| b002509 | + | + | + | − | + | − | + |
| b002510 | − | − | − | − | − | − | − |
| b002511 | + | + | + | + | − | − | + |
| b002512 | − | + | + | − | − | − | + |
| b002513 | + | − | + | − | + | + | + |
| b002514 | − | + | + | − | − | − | + |
| b002515 | + | + | + | + | − | − | + |
| b002516 | + | − | + | − | + | + | + |
| b002517 | + | − | + | + | + | + | + |
| b002518 | + | + | + | − | + | + | + |
| b002519 | − | + | + | − | + | + | + |
| b002520 | + | − | + | − | − | − | + |
| b002521 | − | − | − | + | + | + | + |
| b002522 | + | + | + | + | − | + | + |
| b003247 | + | + | + | − | + | − | + |
| b003347 | − | + | + | − | + | + | + |
| b003348 | + | + | + | + | + | − | + |
| b003349 | + | + | + | + | − | − | + |
| b003350 | + | + | + | + | + | + | + |
| b003351 | − | + | + | − | − | − | + |
| b003352 | − | + | + | − | − | − | + |
| b003353 | − | + | + | − | − | − | + |
| b003354 | + | + | + | + | + | + | + |
| b003355 | − | + | + | + | − | − | + |
| b003356 | + | − | + | + | + | − | + |
| b003357 | + | + | + | + | + | − | + |
| b003358 | − | + | − | − | − | − | + |
| b003359 | − | + | − | + | + | − | + |
| b003360 | + | − | + | − | − | − | + |
| b003361 | + | + | + | + | + | + | + |
| b003362 | − | − | + | + | − | + | + |
| b003363 | + | + | + | + | + | + | + |
| b003364 | − | + | + | − | − | + | + |
| b003365 | − | − | − | − | − | − | − |
| b003366 | + | − | + | − | + | + | + |
| b003367 | − | + | − | + | − | − | + |
| b003368 | − | + | + | − | + | + | + |
| b003369 | + | − | + | + | + | + | + |
| b003370 | + | + | + | + | + | + | + |
| b003371 | + | + | + | + | + | + | + |
| b003372 | + | + | − | + | + | − | + |
| b003373 | − | + | + | − | − | − | + |
| b003374 | + | − | + | − | + | + | + |
| b003375 | + | − | + | + | + | + | + |
| b003376 | − | + | + | − | + | + | + |
| b003377 | + | − | + | − | − | + | + |
| b003378 | − | + | + | + | + | − | + |
| b003379 | − | + | + | − | + | + | + |
| b003380 | − | + | + | − | − | − | + |
| b003381 | + | + | + | − | + | − | + |
| b003382 | − | + | + | − | − | − | + |
| b003383 | − | + | + | + | + | + | + |
| b003384 | + | + | + | + | − | + | + |
| b003385 | − | + | + | − | + | + | + |
| b003386 | + | + | + | + | + | − | + |
| b003387 | + | + | + | + | + | + | + |
| b003388 | + | − | + | − | + | + | + |
| b003389 | − | + | + | + | + | + | + |
| b003390 | − | + | + | − | + | + | + |
| b003391 | + | + | + | + | + | + | + |
| b003392 | + | − | + | − | − | + | + |
| b003393 | + | + | + | − | + | + | + |
| b003394 | + | + | + | + | + | + | + |
| b003395 | + | + | + | + | + | − | + |
| b003396 | + | + | + | − | − | − | + |
| b003397 | + | + | + | − | + | − | + |
| b003398 | + | − | + | + | + | + | + |
| b003399 | + | + | + | + | + | − | + |
| b003400 | + | + | + | + | + | + | + |
| b003401 | + | + | + | + | − | − | + |
| b003402 | + | + | + | − | − | − | + |
| b003403 | + | − | − | + | + | − | + |
| b003404 | + | + | + | − | − | − | + |
| b003405 | − | + | + | + | − | − | + |
| b003406 | + | + | + | − | − | − | + |
| b003407 | + | − | + | − | + | + | + |
| b003408 | + | + | + | + | + | − | + |
| b003409 | + | + | + | + | + | + | + |
| b003410 | − | + | + | + | + | − | + |
| b003411 | + | + | + | + | + | + | + |
| b003412 | + | + | + | − | + | − | + |
| b003413 | + | − | + | + | + | − | + |
| b003414 | + | + | + | + | − | + | + |
| b003415 | + | + | + | + | + | − | + |
| b003416 | − | + | + | − | − | − | + |
| b003417 | + | + | + | + | + | + | + |
| b003418 | + | + | + | − | + | + | + |
| b003419 | + | + | + | − | + | + | + |
| b003420 | − | + | + | + | + | − | + |
| b003421 | + | − | − | − | + | − | + |
| b003422 | + | + | + | + | + | + | + |
| b003675 | + | + | + | − | + | + | + |
| b003762 | − | + | + | − | + | + | + |
| b003763 | + | − | + | − | − | − | + |
| b003764 | + | + | − | + | − | − | + |
| b003765 | + | + | + | + | + | − | + |
| b003770 | + | + | + | + | + | + | + |
| b003771 | − | − | − | + | + | + | + |
| b003773 | − | + | + | − | + | + | + |
| b003774 | + | + | + | − | + | − | + |
| b003777 | − | + | − | + | + | + | + |
| b003779 | + | + | + | + | + | − | + |
| b003780 | − | + | + | − | − | − | + |
| b003781 | + | − | − | − | − | − | − |
| b003782 | + | + | + | + | + | + | + |
| b003783 | + | + | + | + | + | + | + |
| b003786 | + | − | − | + | − | + | + |
| b003787 | + | + | + | + | − | + | + |
| b003788 | + | + | + | + | + | + | + |
| b003790 | − | + | + | − | − | − | + |
| b003791 | + | + | + | + | − | + | + |
| b003793 | + | + | + | + | + | + | + |
| b003795 | − | − | + | + | − | − | + |
| b003798 | − | + | + | − | + | + | + |
| b003802 | − | − | − | − | − | − | − |
| b003804 | − | + | + | + | − | + | + |
| b003805 | − | + | + | + | + | + | + |
| b003806 | − | − | − | + | + | − | + |
| b003812 | + | + | + | + | + | − | + |
| b003813 | + | + | + | + | + | + | + |
| b003815 | + | + | + | + | + | + | + |
| b003818 | + | + | + | + | + | + | + |
| b003821 | − | + | + | + | + | − | + |
| b003823 | + | + | + | + | + | − | + |
| b003826 | − | − | − | − | − | − | − |
| b003827 | − | + | − | − | + | + | + |
| b003829 | − | − | − | − | − | − | − |
| b003830 | − | + | + | − | + | + | + |
| b003838 | − | − | + | + | − | − | + |
| b003840 | + | − | − | − | − | − | + |
| b003841 | + | + | + | − | − | + | + |
| b003843 | + | + | − | − | − | − | + |
| b003845 | + | + | + | + | + | + | + |
| b003850 | − | − | − | + | − | − | − |
| b003851 | − | + | + | − | + | + | + |
| b003852 | + | + | + | + | + | + | + |
| b003853 | + | + | + | + | + | + | + |
| b003855 | + | + | + | + | + | + | + |
| b003856 | − | + | + | − | + | + | + |
| b003864 | + | + | + | − | + | + | + |
| b003865 | + | + | + | + | + | − | + |
| b003868 | + | − | + | + | + | + | + |
| b003871 | + | + | + | + | + | + | + |
| b003872 | − | + | + | + | + | − | + |
| b003874 | − | − | − | + | − | + | − |
| b003880 | + | − | + | − | + | − | + |
| b003885 | + | + | + | + | − | − | + |
| b003886 | − | + | − | + | + | + | + |
| b003887 | + | + | + | + | + | − | + |
| b003889 | + | + | + | + | + | + | + |
| b003890 | + | − | + | − | − | + | + |
| b003893 | + | + | + | + | + | + | + |
| b003894 | − | + | + | − | + | + | + |
| b003896 | − | − | − | + | + | + | + |
| b003897 | − | − | − | − | − | − | − |
| b003898 | + | + | + | − | + | + | + |
| b003899 | − | + | + | − | + | + | + |
| b003900 | − | + | + | − | + | − | + |
| b003902 | + | + | + | + | + | + | + |
| b003903 | + | + | + | − | + | − | + |
| b003907 | − | + | + | − | + | + | + |
| b003908 | − | − | − | + | + | + | + |
| b003909 | − | + | + | + | + | + | + |
| b003912 | + | + | + | + | + | − | + |
| b003920 | + | + | + | − | + | + | + |
| b003922 | − | + | + | − | + | + | + |
| b003923 | + | + | + | − | + | + | + |
| b003924 | − | − | + | − | + | − | + |
| b003926 | + | + | + | + | + | + | + |
| b003930 | − | + | + | − | + | + | + |
| b003931 | + | + | + | − | + | + | + |
| b003932 | − | + | + | − | + | + | + |
| b003934 | + | + | + | − | + | + | + |
| b003935 | − | − | − | − | − | − | − |
| b003941 | − | + | + | − | + | + | + |
| b003942 | + | + | + | + | + | + | + |
| b003943 | − | − | − | + | + | − | + |
| b003944 | + | + | + | − | − | − | + |
| b003945 | − | + | + | − | + | + | + |
| b003947 | + | − | + | + | + | − | + |
| b003948 | − | − | − | − | − | − | − |
| b003949 | + | + | + | + | − | − | + |
| b003952 | + | + | + | − | + | + | + |
| b003954 | − | − | − | − | − | − | − |
| b003956 | + | + | + | + | − | − | + |
| b003957 | − | + | + | + | − | − | + |
| b003958 | + | + | + | + | − | − | + |
| b003962 | + | + | + | − | + | − | + |
| b003963 | + | + | + | − | + | − | + |
| b003965 | + | + | + | − | − | + | + |
| b003969 | + | − | + | − | + | + | + |
| b003970 | − | + | + | − | + | + | + |
| b003974 | − | + | + | − | + | + | + |
| b003976 | + | + | + | + | − | − | + |
| b003977 | + | − | + | + | + | − | + |
| b003978 | + | − | − | + | + | + | + |
| b003979 | + | + | + | + | + | + | + |
| b003982 | + | + | + | + | + | + | + |
| b003983 | + | − | − | − | + | + | + |
| b003984 | + | − | + | + | − | − | + |
| b003986 | − | + | + | + | + | + | + |
| b003989 | + | + | + | + | + | − | + |
| b003990 | − | − | + | − | + | + | + |
| b003996 | + | − | + | + | + | − | + |
| b003998 | + | + | + | + | + | + | + |
| b004000 | − | + | + | − | + | + | + |
| b004008 | − | − | − | − | − | − | − |
| b004009 | + | − | + | + | − | − | + |
| b004012 | − | + | + | − | + | + | + |
| b004013 | − | + | − | + | − | + | + |
| b004014 | + | + | + | − | + | + | + |
| b004015 | − | + | + | − | + | + | + |
| b004016 | − | + | + | − | + | + | + |
| b004017 | + | + | + | + | + | + | + |
| b004018 | + | + | + | − | + | − | + |
| b004020 | + | − | + | + | − | + | + |
| b004021 | + | + | + | − | − | − | + |
| b004022 | − | − | − | − | − | − | − |
| b004023 | − | + | + | − | + | + | + |
| b004025 | − | − | − | + | − | − | − |
| b004026 | + | + | + | + | − | + | + |
| b004027 | + | + | + | + | + | − | + |
| b004028 | − | + | + | − | + | + | + |
| b004031 | + | + | + | + | + | + | + |
| b004032 | − | − | − | − | − | − | − |
| b004033 | − | − | + | + | + | − | + |
| b004034 | − | − | − | + | + | + | + |
| b004037 | − | + | + | − | − | − | + |
| b004038 | − | − | − | + | − | − | + |
| b004039 | + | − | + | + | + | − | + |
| b004040 | − | + | − | + | + | + | + |
| b004041 | − | + | + | + | + | + | + |
| b004043 | − | − | − | + | + | − | + |
| b004044 | + | + | + | + | + | + | + |
| b004045 | + | − | + | − | + | + | + |
| b004046 | + | + | + | + | + | + | + |
| b004048 | + | + | + | − | + | + | + |
| b004049 | − | + | + | + | + | + | + |
| b004052 | + | + | + | + | + | − | + |
| b004057 | + | + | + | − | + | + | + |
| b004058 | + | + | + | − | − | − | + |
| b004059 | − | + | − | + | + | + | + |
| b004060 | − | + | + | − | + | + | + |
| b004092 | + | + | + | + | + | + | + |
| b004096 | + | + | + | + | + | − | + |
| b004097 | − | − | − | − | − | − | − |
| b004098 | + | + | + | + | + | − | + |
| b004099 | + | + | + | + | + | + | + |
| b004100 | + | + | − | + | + | − | + |
| b004101 | − | + | + | + | + | + | + |
| b004102 | − | − | − | + | + | − | + |
| b004103 | − | − | − | + | − | − | − |
| b004104 | − | − | − | + | − | − | − |
| b004105 | − | + | + | − | + | + | + |
| b004106 | + | + | + | + | − | − | + |
| b004107 | − | − | − | + | − | − | + |
| b004108 | − | − | − | − | − | − | − |
| b004109 | − | − | − | − | − | − | − |
| b004110 | + | − | + | + | + | − | + |
| b004111 | + | + | + | + | + | + | + |
| b004112 | − | − | − | + | + | + | + |
| b004113 | − | − | − | − | − | − | − |
| b004114 | − | + | + | + | − | − | + |
| b004115 | + | − | − | + | + | − | + |
| b004116 | − | − | − | + | + | − | + |
| b004117 | − | − | + | + | + | + | + |

### Example 11. High Titer Liquid and Plaquing Host Range for Cocktail CK618

The objective of this study was to determine the total host range of CK618 and its component individual bacteriophages by two distinct methods.

### Materials and Methods

A clinically relevant 300-isolate Clinical Panel (CP) was developed. Escherichia coli (E. coli) isolates were sourced from the International Health Management Associates (IHMA) and metadata regarding various characteristics of each strain was obtained. Strains were defined as MDR if they were resistant to three or more classes of antibiotics summarized in Table 14.

TABLE 14
Host Range Panel Metadata
Regional distribution

| Region | # isolates | % of total panel¹ |
|---|---|---|
| North America | 127 | 42.3 |
| Europe | 86 | 29 |
| Latin America | 44 | 15 |
| Asia | 43 | 14 |

Country distribution

| Country | # isolates | % of total panel¹ |
|---|---|---|
| United States | 127 | 42.3 |
| Turkey | 21 | 7.0 |
| Argentina | 21 | 7.0 |
| Russia | 19 | 6.3 |
| Brazil | 18 | 6.0 |
| Japan | 11 | 3.7 |
| Ukraine | 10 | 3.3 |
| Hong Kong | 10 | 3.3 |
| Taiwan | 9 | 3 |
| Italy | 9 | 3 |
| Spain | 8 | 3 |
| Thailand | 8 | 3 |
| Germany | 7 | 2 |
| France | 6 | 2 |
| United Kingdom | 6 | 2 |
| Malaysia | 3 | 1 |
| Mexico | 2 | 0.7 |
| Guatemala | 2 | 0.7 |
| South Korea | 1 | 0.3 |
| Chile | 1 | 0.3 |
| Vietnam | 1 | 0.3 |

State distribution of US isolates

| State | # isolates | % of US isolates¹ |
|---|---|---|
| California | 47 | 37 |
| Florida | 28 | 22 |
| South Carolina | 15 | 12 |
| Alabama | 11 | 8.7 |
| Georgia | 7 | 6 |
| New York | 6 | 5 |
| North Carolina | 3 | 2 |
| Minnesota | 3 | 2 |
| Indiana | 3 | 2 |
| Nebraska | 2 | 2 |
| Ohio | 2 | 2 |

Age distribution

| Age range | # isolates | % of total panel¹ |
|---|---|---|
| 0 to 10 | 10 | 3.3 |
| 11 to 20 | 12 | 4.0 |
| 21 to 30 | 12 | 4.0 |
| 31 to 40 | 25 | 8.3 |
| 41 to 50 | 28 | 9.3 |
| 51 to 60 | 27 | 9.0 |
| 61 to 70 | 53 | 18 |
| 71 to 80 | 62 | 21 |
| 81 to 90 | 42 | 14 |
| 91 to 100 | 11 | 3.7 |
| not specified | 18 | 6.0 |

Gender distribution

| Gender | # isolates | % of total panel¹ |
|---|---|---|
| Female | 186 | 62.0 |
| Male | 114 | 38.0 |

Sampling time distribution

| Collection year | # isolates | % of total panel¹ |
|---|---|---|
| 2017 | 30 | 10. |
| 2018 | 3 | 3 |
| 2019 | 259 | 86.3 |
| 2020 | 2 | 0.7 |

Sampling site distribution

| Body location | # isolates | % of total panel¹ |
|---|---|---|
| Urine | 254 | 84.7 |
| Blood | 26 | 8.7 |
| Bladder | 17 | 5.7 |
| Urethra | 2 | 0.7 |
| Kidneys | 1 | 0.3 |

MDR distribution

| MDR status² | # isolates | % of total panel¹ |
|---|---|---|
| MDR | 115 | 38.3 |
| not MDR | 185 | 61.7 |

¹Due to rounding, not all percentages will sum to 100%.
²MDR = multidrug-resistant. Strains were defined as MDR if they were resistant to three or more classes of antibiotics.

Bacteria plates for both host range assays were created by growing the bacterial strains to log phase for approximately 4 to 6 hours in a 37° C. shaking incubator. Those cultures were then diluted in LB with glycerol to a target final optical density at 600 nm (OD₆₀₀) of 0.02 in 20% glycerol before the panels were frozen. After further testing, the average titer of strains on the panel was determined to be 2.89×10⁶ colony-forming unit (CFU)/mL. The panels were stored in the −80° C. freezer until use for host range testing.

Individual bacteriophages from CK618 were tested at maximum available titer. Additionally, each bacteriophage in CK618; Table 15) was combined into cocktails with three different bacteriophage concentrations.

TABLE 15
Summary of CK618

| | | CK618 | |
|---|---|---|---|
| | | Engineering | Titer |
| Phage | Genus | Status | (PFU/mL) |
| p00exe014 | Tequatrovirus | Full Construct | 5.0 × 10¹⁰ |
| p004Ke009 | Tequatrovirus | Full Construct | 2.1 × 10¹⁰ |
| p00jc | Phapecoctavirus | Wild Type | 2.7 × 10¹¹ |
| p00ke | Unclassified Myovirus | Wild Type | 3.2 × 10¹⁰ |
| p00c0e030 | Mosigvirus | Full Construct | 4.8 × 10¹⁰ |
| p5516 | Vectrevirus | Wild Type | 3.7 × 10¹⁰ |

For the highest titer cocktail, an equal volume of each bacteriophage was combined to produce a cocktail with a total cocktail titer of 1.0×10¹¹ PFU/mL. For two lower titer cocktails, the bacteriophage titers were normalized before combination to produce cocktails with each bacteriophage at a titer of 3.0×10⁹ PFU/mL (total cocktail titer of 1.8×10¹⁰ PFU/mL) or 1.0×10⁷ PFU/mL (total cocktail titer of 6.0×10⁷ PFU/mL). bacteriophages were produced through the process development (PD) process. The PD process uses the same general manufacturing process used for clinical trial material manufacturing except at a smaller scale. Both potency and purity are evaluated for consistency with clinical trial material including endotoxin levels. The cocktails were then subjected to host range testing via a liquid-based assay and a plaquing-based assay on the 300-isolate CP described above with an additional 4 strains of interest.

### Liquid-Based Host Range

Frozen bacterial panels were pulled from the freezer at least 45 minutes before the start of the Liquid Host Range protocol on the Hamilton VANTAGE liquid handling robot. The instrument was prepared with all of the necessary consumables, including LB amended with 10 mM CaCl₂ and 10 mM MgCl₂ (hereafter referred to as LB+salts) as the growth medium, the bacteria panels (up to 4 different panels in 96 well microtiter plates), and bacteriophage lysates in Eppendorf tubes. Blank samples with LB+salts only were included in the 96-well microtiter plates and in the Eppendorf tubes to test for potential contamination of the run and as a control. Each well of a 96-well microtiter plate was loaded by the liquid-handling robot with 40 μL of LB+salts, then 5 μL of bacteriophage, then 5 μL of bacteria from the bacterial panel. This preparation produced a final bacteriophage concentration in the test sample 1/10 the concentration of the input bacteriophage. Once all combinations were prepared, the plates were put into the shaking incubator at 37° C. Plates were incubated for 20 hours, and the OD₆₀₀ of each well in each plate was read every hour.

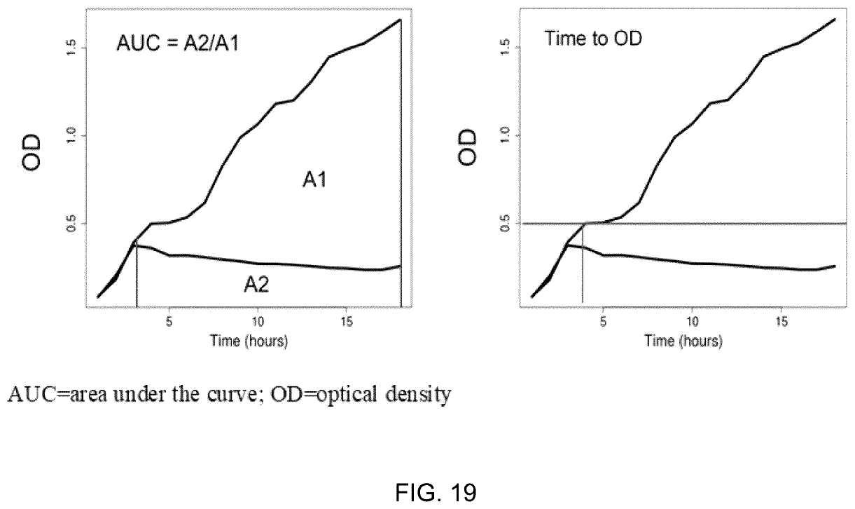

The aforementioned protocol generates growth curves for each bacterial strain in the presence and absence of the bacteriophage cocktail. The area under the curve (AUC) is computed, and the ratio of (AUC in the presence of bacteriophage)/(AUC in the absence of bacteriophage) is calculated (FIG. 19, left panel). If the AUC ratio is <0.65, the strain is considered to be targeted by the bacteriophage cocktail. The number of strains targeted divided by the number of strains tested gives the host range percentage, or % HR. The same analysis was performed on the subset of strains that are classified as multidrug-resistant (MDR). Additionally, some strains that are targeted by the bacteriophage cocktail eventually develop resistance and begin to grow, thus increasing the OD₆₀₀ reading in that well. Other strains remain suppressed until the end of the assay time course (20 hours). Strain growth was quantified by examining the amount of time in hours for a given strain+bacteriophage sample to reach an OD₆₀₀ of 0.4 (FIG. 19, right panel). If the sample never reached an OD₆₀₀ of 0.4, that sample was considered suppressed. The number of strains that remain suppressed to below an OD₆₀₀ of 0.4 divided by the number of strains tested gives the suppression metric % OD<0.4.

### Plaquing-Based Host Range

Frozen bacterial panels were removed from the freezer to thaw, and 1 mL of LB was added to each well of the deep well block to incubate overnight in a 37° C. shaking incubator. Once the cultures had grown overnight, they were used to produce the overlays needed for the double agar overlay method. To create these overlays, rectangular LB agar plates were used with an overlay mixture of 100 μL of bacteria and 0.56% low salt top agar (low salt top agar mixed 3:1 with LB) poured over the plate. The overlays were poured onto the agar plates and left to solidify for at least 20 minutes before spotting.

Phage plaquing was performed using the Hamilton VANTAGE liquid handling robots. Each bacteriophage (at maximum available titer, Table 15) or bacteriophage cocktail (as described in “Materials and Methods” section) was serially diluted 1:10 in LB. After solidification of the double agar overlay, bacteriophage were spotted onto the overlay by the robots. Once spotting was completed and the spots had dried, the plates were taken from the liquid handling robot and placed in a 37° C. incubator overnight.

After an overnight incubation, the plates were imaged using an integrated camera, IDS uEye, on the liquid handling robot Hamilton STAR. An in-house algorithm was used to identify sensitive bacterial strains by counting the number of individual plaque forming units. A minimum of three plaque forming units was used to designate a hit. Plates giving outlier results (>2 log difference between three methods in the algorithm) and plates with no plaques detected by the algorithm were counted manually.

### Results

Host range of the bacteriophage cocktail was tested using two methods which generate AUC after bacteriophage exposure in liquid and plaquing on bacterial overlays. The combination of these two metrics demonstrated that at max feasible titer CK618 targets 92.4% of the strains in the CP. On the MDR strain subset which represents 38.8% of the CP, the host range percentage increased to 94.1% for CK618.

An additional metric used to determine a cocktail's performance is Time to OD. This metric is useful for determining how effective a cocktail is at suppressing growth of the bacterial strains after the initial decrease. The score considers the percentage of strains to not rebound to OD ≥0.4 after the initial decrease in OD. Therefore, a higher Time to OD percentage represents those strains that were suppressed by the cocktail. The Time to OD score for CK618 was 67.8% at max feasible titer on the full panel and 62.4% for MDR strains (Table 16).

TABLE 16
Host range of CK507, CK618, and individual bacteriophages by different measurement methods

| | | p00exe014 | p004ke009 | p00c0e030 | p00ke | p00jc | p5516 | CK618 | CK618 | CK618 |
|---|---|---|---|---|---|---|---|---|---|---|
| | | 5.0 × 10⁹ | 2.1 × 10⁹ | 4.8 × 10⁹ | 3.2 × 10¹⁰ | 2.7 × 10¹⁰ | 4.3 × 10⁸ | 1.0 × 10¹⁰ | 1.8 × 10⁹ | 6.0 × 10⁶ |
| Metric | Measurement | PFU/mL | PFU/mL | PFU/mL | PFU/mL | PFU/mL | PFU/mL | PFU/mL | PFU/mL | PFU/mL |
| % HR | Liquid | 67.9 | 39.5 | 4.3 | 37.7 | 38.7 | 24.8 | 82.9 | 78.0 | 71.7 |
| | Plaquing | 77.3 | 51.3 | 71.7 | 47.7 | 51.6 | 38.8 | 91.5 | 91.5 | 91.5 |
| | Liquid OR | 81.6 | 57.2 | 71.7 | 67.1 | 55.3 | 54.4 | 93.6 | 92.8 | 92.2 |
| | plaquing | | | | | | | | | |
| % OD < 0.4 | Liquid | 49.8 | 20.9 | 1.2 | 18.4 | 16.4 | 4.9 | 67.8 | 59.2 | 50.5 |
| MDR % HR | Liquid | 63.4 | 26.9 | 2.7 | 37.1 | 32.3 | 26.3 | 82.8 | 74.2 | 69.4 |
| | Plaquing | 78.5 | 37.6 | 72.0 | 31.2 | 33.3 | 41.9 | 90.3 | 90.3 | 90.3 |
| | Liquid OR | 81.7 | 42.5 | 72.0 | 55.9 | 39.3 | 60.8 | 94.1 | 92.5 | 90.3 |
| | plaquing | | | | | | | | | |
| MDR % | Liquid | 49.5 | 12.4 | 1.1 | 19.4 | 12.9 | 7.5 | 62.4 | 54.8 | 51.1 |
| OD < 0.4 | | | | | | | | | | |

### Example 12. In Vitro Synergy with Antibiotics

The objective of this study was to assess the interaction of CK618 with antibiotics.

### Materials and Methods

Phage cocktail CK618 was tested in combination with and alongside antibiotics against two isolate panels. The first assay used a panel of 88 contemporaneous urinary tract infection (UTI) isolates from North America, Europe, Latin America, and Asia. Twenty-one (24%) of those isolates are classified as multidrug-resistant (MDR), with resistance to 3 or more antibiotic classes. Seven different antibiotics were tested against this panel, representing five standard-of-care (SOC) antibiotics (ceftriaxone, cephalexin, trimethoprim/sulfamethoxazole [TMP/SMX; also called cotrimoxazole], fosfomycin, and nitrofurantoin) and two non-SOC antibiotics (cefdinir and ciprofloxacin) that are commonly used as comparators. The second assay used a panel made up of a 300-strain clinical isolate panel (Table 14) with an additional 4 strains of interest. Ninety-three (31%) of those isolates are classified as MDR. Four of the same SOC antibiotics tested against the 88-isolate panel were tested against this panel. The non-SOC antibiotics were not included due to their limited use in clinical settings, and nitrofurantoin was not included due to the low probability of resistance to this drug.

For both isolate panels, antibiotic concentrations tested were determined by using Clinical and Laboratory Standards Institute (CLSI) or European Committee on Antimicrobial Susceptibility Testing (EUCAST) breakpoints at concentrations that kill susceptible isolates but allow intermediate or resistant isolates to grow in the standard AST microdilution assay (Table 17).

TABLE 17
Antibiotic and bacteriophage Treatment Conditions

| Antibiotic or | | Concentration | | Breakpoint |
|---|---|---|---|---|
| bacteriophage Cocktail | Class | in Assay | | Reference |
| Cefdinir | 3^{rd} gen cephalosporin | 1 | μg/ml | CLSI M100 Table 2A |
| Ceftriaxone | 3^{rd} gen cephalosporin | 1 | μg/ml | CLSI M100 Table 2A |
| Cephalexin | 1^{st} gen cephalosporin | 16 | μg/ml | EUCAST Breakpoint |
| | | | | Table v 11.0 |
| Ciprofloxacin | Fluoroquinolone | 0.25 | μg/ml | CLSI M100 Table 2A |
| Trimethoprim/ | Folate pathway | 2 | μg/ml | CLSI M100 Table 2A |
| Sulfamethoxazole | | | | |
| (TMP/SMX) | | | | |
| Fosfomycin | Fosfomycin | 8 | μg/ml | EUCAST Breakpoint |
| | | | | Table v 11.0 |
| | | | | (uncomplicated UTI only) |
| Fosfomycin | Fosfomycin | 64 | μg/ml | CLSI M100 Table 2A |
| Nitrofurantoin | Nitrofurans | 32 | μg/ml | CLSI M100 Table 2A |

| | | | |
|---|---|---|---|
| LBP-EC01 | Bacteriophage Cocktail | 6 × 10⁶ PFU/ml total, | Not applicable |
| | | 1 × 10⁶ PFU/ml per | |
| | | bacteriophage | |

CLSI = Clinical and Laboratory Standards Institute;
EUCAST = European Committee on Antimicrobial Susceptibility Testing;
PFU = plaque-forming unit;
SMX = sulfamethoxazole;
TMP = trimethoprim

The composition of the proposed Phase 2 CRISPR-enhanced bacteriophage (CRISPR-phage, hereafter termed ‘crPhage’) cocktail (CK618) has been updated from the CK570 to include six bacteriophages as compared to three bacteriophages in CK570. Bacteriophage cocktail CK618, was prepared such that all bacteriophages had equivalent titers in the final cocktail. The cocktail was then mixed with bacteria to obtain final titers of 1×10⁶ plaque-forming unit (PFU)/mL/phage for the cocktail and 1×10⁵ colony-forming unit (CFU)/mL for the bacteria, resulting in a multiplicity of infection (MOI) of 10 for each bacteriophage in a sample. Briefly, 5 μl of each sample (CK618 alone, CK618 and antibiotics, antibiotics alone, or medium alone) was added to 5 μl of optical density (OD)-normalized bacterial isolate and 40 μl LB medium. Reactions were incubated at 37° C. with shaking and OD measurements were taken each hour for 20 hours.

Host range percentage is the percent of isolates that responded to treatment of antibiotics or CK618. In the case of the 88-isolate panel, results were analyzed on the complete panel as well as the subset of classified as multidrug-resistant (MDR) (n=21) and beta-lactam-resistant (n=31). In the case of the 304-isolate panel, results were analyzed on the complete panel as well as the subset of isolates classified as MDR (n=93) and beta-lactam-resistant (n=91).

### Results

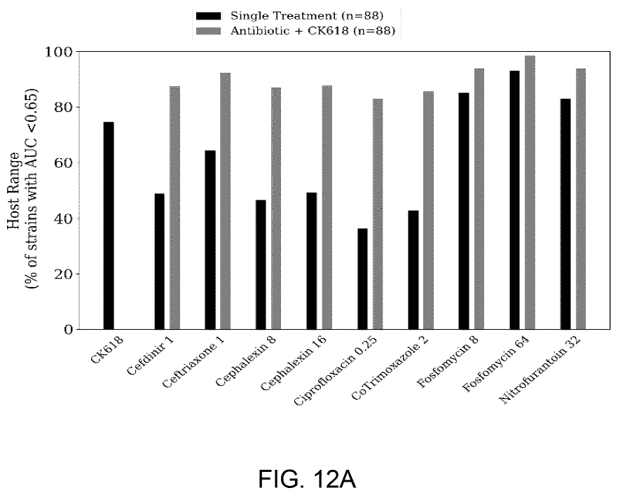

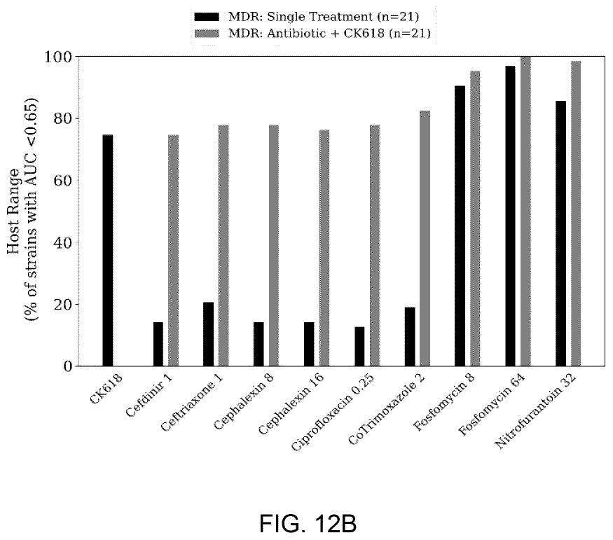

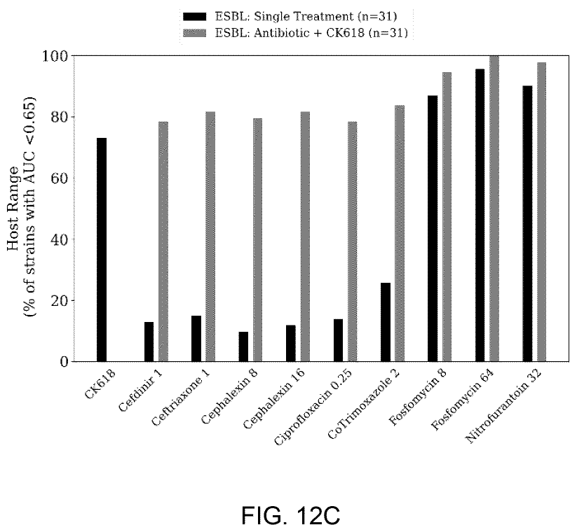

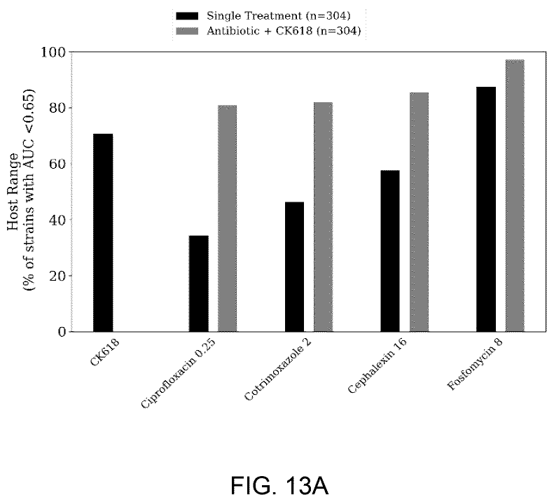

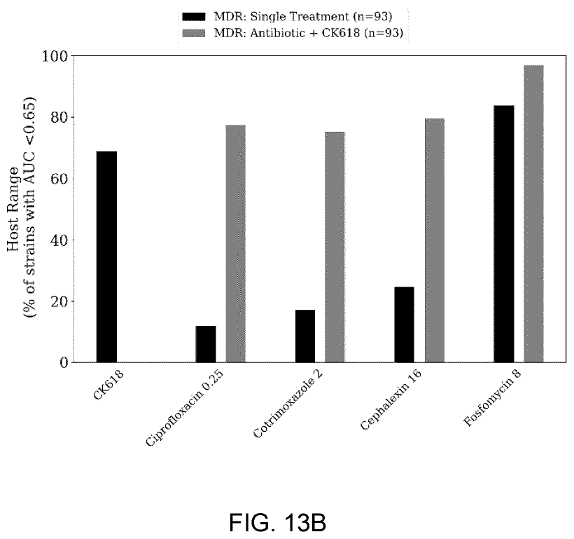

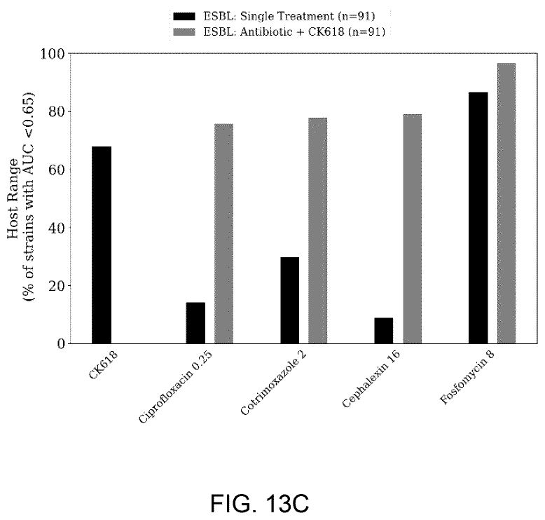

CK618 outperformed most SOC antibiotics, including first and third generation cephalosporins, ciprofloxacin, and TMP/SMX on all strains (FIGS. 12A-12C, FIGS. 13A-13C, Tables 18, 19). Combination therapy was more effective in all cases compared with antibiotic treatment alone. In the MDR and beta-lactam-resistant subsets of strains, combination therapy with the cephalosporins, ciprofloxacin, or TMP/SMX performs similarly to, or slightly better than, the bacteriophage cocktail alone. Fosfomycin and nitrofurantoin had strong activity across all strains and were minorly improved by the addition of LBP-EC01. For MDR and beta-lactam-resistant stains, the overall host range of cephalosporins, ciprofloxacin, and TMP/SMX was reduced while LBP-EC01, Nitrofurantoin, and fosfomycin performed similarly regardless of antibiotic resistance status across the isolates tested (FIGS. 12A-12C, FIGS. 13A-13C, Tables 18, 19).

TABLE 18
Host Range of CK618 and Antibiotics Alone and in Combination on 88-Isolate Panel

| | Host Range % on all | | Host Range % on | | Host Range % on Beta-lactam- | |
|---|---|---|---|---|---|---|
| | Strains (n = 88) | | MDR Subset (n = 21) | | Resistant Subset (n = 31) | |
| | | Combination: | | Combination: | | Combination: |
| | Single agent | Antibiotic + | Single agent | Antibiotic + | Single agent | Antibiotic + |
| Treatment | treatment | CK618 v.2 | treatment | CK618 v.2 | treatment | CK618 v.2 |
| CK618 | 74.6 | n/a | 74.6 | n/a | 73.1 | n/a |
| Cefdinir | 48.9 | 87.5 | 14.3 | 74.6 | 12.9 | 78.5 |
| Ceftriaxone | 64.4 | 92.4 | 20.6 | 77.8 | 15.1 | 81.7 |
| Cephalexin | 49.2 | 87.9 | 14.3 | 76.2 | 11.8 | 81.7 |
| Ciprofloxacin | 36.4 | 83.0 | 12.7 | 77.8 | 14.0 | 78.5 |
| TMP/SXT | 42.8 | 85.6 | 19.0 | 82.5 | 25.8 | 83.9 |
| Fosfomycin 8 | 85.2 | 93.9 | 90.5 | 95.2 | 87.1 | 94.6 |
| Fosfomycin 64 | 93.2 | 98.5 | 96.8 | 100.0 | 95.7 | 100.0 |
| Nitrofurantoin | 83.0 | 93.9 | 85.7 | 98.4 | 90.3 | 97.8 |

MDR = multidrug-resistant

TABLE 19
Host Range of CK618 and Antibiotics Alone and in Combination on 304-Isolate Panel

| | Host range % on all | | Host range % on | | Host range % on beta-lactam- | |
|---|---|---|---|---|---|---|
| | strains (n = 304) | | MDR subset (n = 93) | | resistant subset (n = 91) | |
| | | Combination: | | Combination: | | Combination: |
| | Single agent | Antibiotic + | Single agent | Antibiotic + | Single agent | Antibiotic + |
| Treatment | treatment | LBP-CK618 | treatment | LBP-CK618 | treatment | LBP-CK618 |
| CK618 | 70.7 | n/a | 68.8 | n/a | 68.1 | n/a |
| Ciprofloxacin 0.25 | 34.2 | 80.9 | 11.8 | 77.4 | 14.3 | 75.8 |
| Cotrimoxazole 2 | 46.4 | 81.9 | 17.2 | 75.3 | 29.7 | 78.0 |
| Cephalexin 16 | 57.6 | 85.5 | 24.7 | 79.6 | 8.8 | 79.1 |
| Fosfomycin 8 | 87.5 | 97.0 | 83.9 | 96.8 | 86.8 | 96.7 |

These data demonstrate that CK618 outperformed all of the antibiotics tested with the exception of the revived drug Fosfomycin, which already has a few demonstrated cases of antibiotic resistance mechanisms and is efficacious currently only because it has not been widely used until the emergence of MDR/XDR isolates. Additionally, CK618 acts in a complementary fashion with antibiotics. Regardless of isolate MDR status or antibiotic identity, in all cases concurrent treatment with CK618 plus antibiotic demonstrates a higher host range than treatment with either CK618 or antibiotic alone. In the case of Bactrim (TMP/SMX or cotrimoxazole), the percent of MDR strains from the 304-isolate panel targeted increases from 17.2% when treated with Bactrim alone to 75.3% when treated with Bactrim+CK618 (Table 19), providing a strong argument for the benefit of concurrent treatment.

### Example 13. Urinary Tract Infection Model to Test Cocktail Efficacy

A urinary tract infection (UTI) model was developed in C3H/OuJ mice infected with Escherichia coli (E. coli) strain ATCC 700928 that resulted in stable infection of the bladder and kidneys with E. coli. CK618 surrogate cocktail CK570 was tested in this study to determine the extent to which the cocktail can reduce E. coli burden in bladder, kidneys, and urine.

### Materials

The CRISPR bacteriophage (‘crPhage’) cocktail included 6 bacteriophages (p004k, p00jc, p00c0, p00ke, p5516 and p00ex). The full construct version of p00jc used in CK570 uses Cpf1/Cas12a as the CRISPR system rather the PAIC Cas operon used in other full construct bacteriophages, and was produced through the process development (PD) process. The PD process uses the same general manufacturing process used for clinical trial material manufacturing except at a smaller scale. Both potency and purity are evaluated for consistency with clinical trial material including endotoxin levels.

TABLE 22
Components of Surrogate Cocktail CK570

| Phage | Genus | Engineering Status |
|---|---|---|
| p00exe014 | Tequatrovirus | Full Construct |
| p004ke009 | Tequatrovirus | Full Construct |
| p00jce006 | Phapecoctavirus | Full Construct¹ |
| p00ke | Unclassified Myovirus | Wild Type |
| p00c0e030 | Mosigvirus | Full Construct |
| p5516 | Vectrevirus | Wild Type |

¹The full construct version of p00jc used in CK570 uses Cpf1/Cas12a as the CRISPR system rather than PAIC Cas operon used in the other full construct bacteriophages.

CK570 was formulated in 1×tris buffered saline (TBS), with a composite potency of 7.6×10¹⁰ plaque-forming units (PFU)/mL and an estimated endotoxin content of 16.6 EU/mL. Mice that were treated with CK570 received 3.8×10⁹ PFU/dose via intraurethral (IU) administration or 7.6×10⁹ PFU/dose via IV administration. The cocktail potency relative to the infection strain was 2.0×10¹⁰ PFU/mL (as determined by serial dilution and plating on ATCC 700928 bacterial overlays). The vehicle used in this study was 1×TBS. Ciprofloxacin was a positive control and was prepared as an injectable by a compounding pharmacy as a 2.5 mg/mL stock in water.

C3H/OuJ female mice, aged approximately 8 weeks and weighing 21 to 24 grams, were used in this study. Mice were housed with 1 to 5 animals per cage and were provided food (5P76 Prolab isopro irradiated lab diet) and water ad libitum.

### Experimental Design

On Day 0, animals in Groups 1 through 5 received a single IU dose of 50 μL of E. coli (as described below). Forty-eight (48), 60, 72, 84, and 96 hours post-infection (p.i.), all animals received vehicle (1×TBS), CK570, or ciprofloxacin administered IU (Groups 1 and 2) or IV (Groups 3 through 5) (Table 20)

TABLE 20
Experimental design

| | | Day 0 Infection | 48 hours Post-infection Treatment | | | |
|---|---|---|---|---|---|---|
| | | Bacteria, Dose, | Test Article | ROA and | Dosing | Experimental |
| Group | # | ROA, & Volume | and Dose | Volume | Schedule | Endpoints |
| 1 | 15 | E. coli | TBS | IU 50 μL | 48, 60, 72, | Urine, bladder, |
| 2 | 15 | (b3675) | CK570 7.6 × | | 84 and 96 | blood, kidneys, |
| | | 1 × 10⁹ CFU | 10¹⁰ PFU/mL | | hours post- | and spleen |
| 3 | 15 | IU | TBS | IV 100 μL | infection | were collected |
| 4 | 15 | 50 μL | CK570 7.6 × | | | from n = 5 |
| | | | 10¹⁰ PFU/mL | | | mice/timepoint |
| 5 | 15 | | Ciprofloxacin | | | |
| | | | 5 mg/mL | | | |

CFU = colony forming unit; IU = intraurethral; IV = intravenous; PFU = plaque forming unit; ROA = route of administration; TBS = tris buffered saline. Cocktail dose is listed as composite potency.

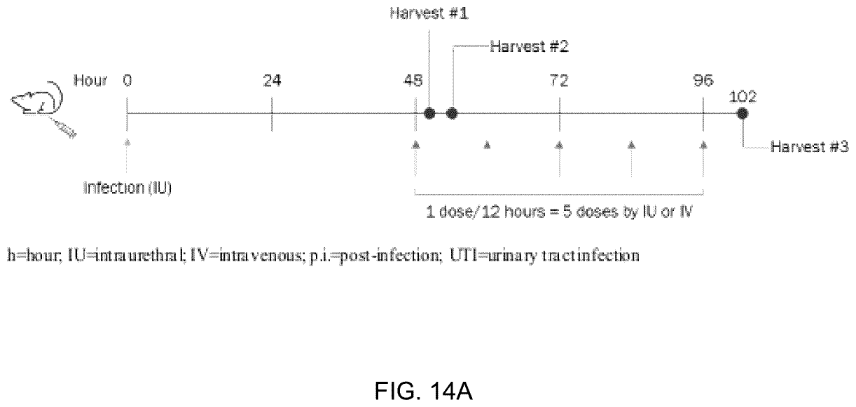

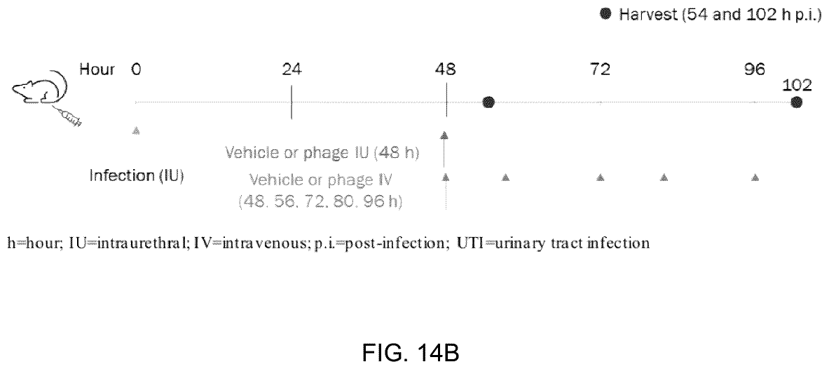

Briefly, on Day 0, Time 0, mice were infected with E. coli (ATCC 700928) at 109 colony-forming units (CFU) by IU administration (FIG. 14A). Mice were anesthetized with 1-4% isoflurane in oxygen for 2-3 minutes until a deep plane of anesthesia was achieved, confirmed by toe pinch reflex. Each mouse was then placed on a board, ventral side up, with the nose inserted into a nose cone supplying isoflurane. The lower abdomen was gently massaged to expel any urine in the bladder. Using a 24 G neonatal catheter (BD Insyte-N Autoguard BC 24 GA 0.561N [0.7×14 mm] cat #381411) connected to a 1 mL syringe, 50 μL of inoculum was slowly instilled into the bladder. For IV administration, mice were manually restrained in a restrainer device, and study treatment was administered via the lateral tail vein. A dose volume of 100 μL was used for IV injections. For IU administration, the same method was used as for the original administration of E. coli. A dose volume of 50 μL was used for IU administration.

Two cohorts of mice were then euthanized, and E. coli burden was assessed by serial dilution and culture from homogenized tissues at 49 hours and 54 hours p.i. (1 hour and 6 hour post-treatment, respectively). A third cohort of mice received 4 additional doses of either vehicle, ciprofloxacin, or CK570 every 12 hours until 96 hours p.i. At 102 hours p.i. (6 hours post-final treatment), the remaining mice were euthanized, and E. coli burden enumerated. Following anesthesia with inhaled isoflurane (1-5%), the abdomen was opened to expose the bladder and urine was aseptically collected using an insulin syringe and placed into a sterile, DNAse/RNAse-free tube.

Following urine collection, the bladder was removed and placed into a pre-weighed homogenization tube containing 1.4 mm ceramic beads (Fisher Scientific cat #15-340-153) and 200 μL of sterile phosphate buffered saline (PBS; Teknova cat #P0200) and re-weighed to the thousandths place. Kidneys and spleen were collected in a manner similar to bladder into a volume of 1 mL of sterile PBS. Blood was collected by inserting a needle into the heart and collecting into a 4 mL sodium heparin blood collection tube (Fisher Scientific cat #02-689-5) and gently rotating to ensure adequate mixing with anti-coagulant. Tissues were homogenized, and all samples immediately serially diluted and plated for CFU enumeration. CFU/mL calculations were normalized to gram tissue for bladder and kidneys. All CFU data were log-transformed.

### Results

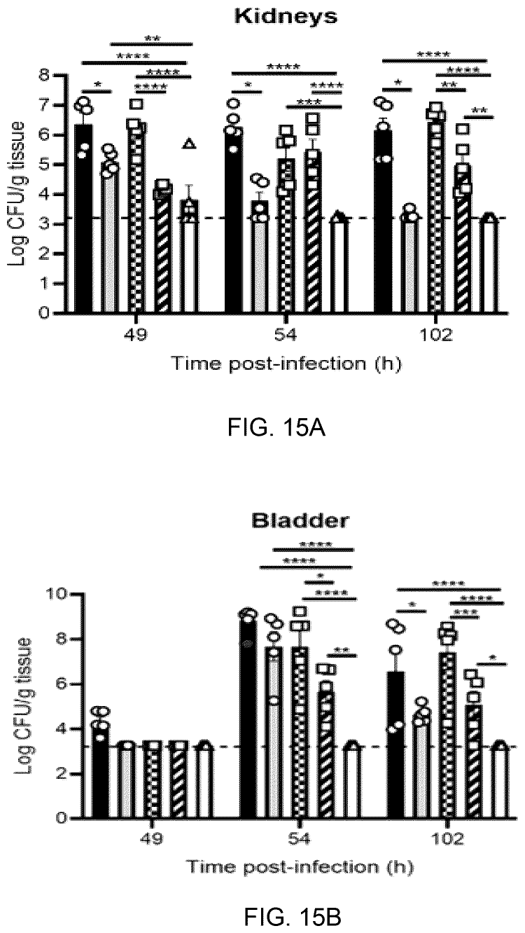

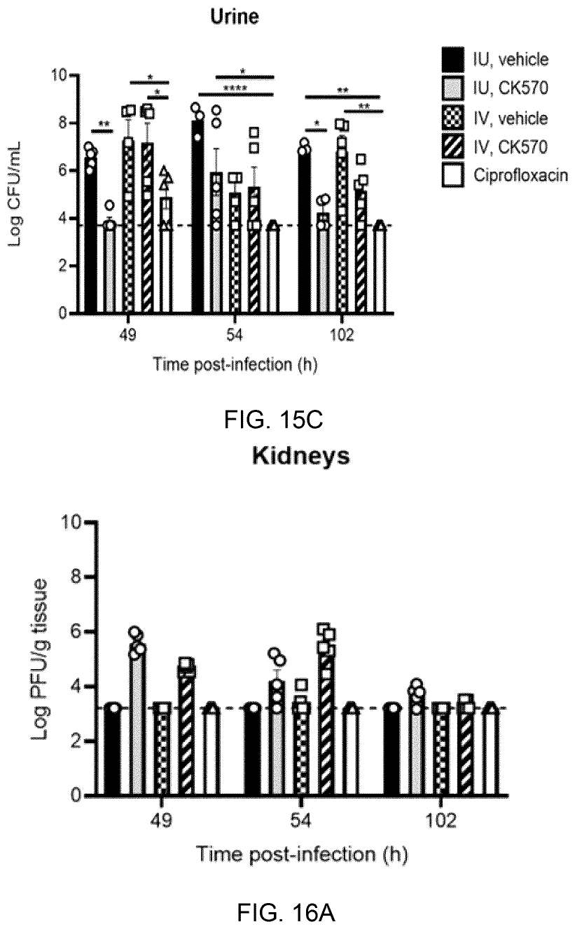

IU administration of CK570, which directly delivered the cocktail to the site of infection, resulted in significant reduction of bacterial burden in kidneys at all timepoints, in bladder at 54 hours p.i. and 102 hours p.i., and in urine at 49 hours p.i. and 102 hours p.i. (FIGS. 15A-15C). Reduction in bacterial burden ranged from ˜2 log to ˜4 log, and in some cases reached the limit of detection (LOD). These data also demonstrate that significant efficacy is achievable with either a single dose or with 5 doses given twice daily (BID).

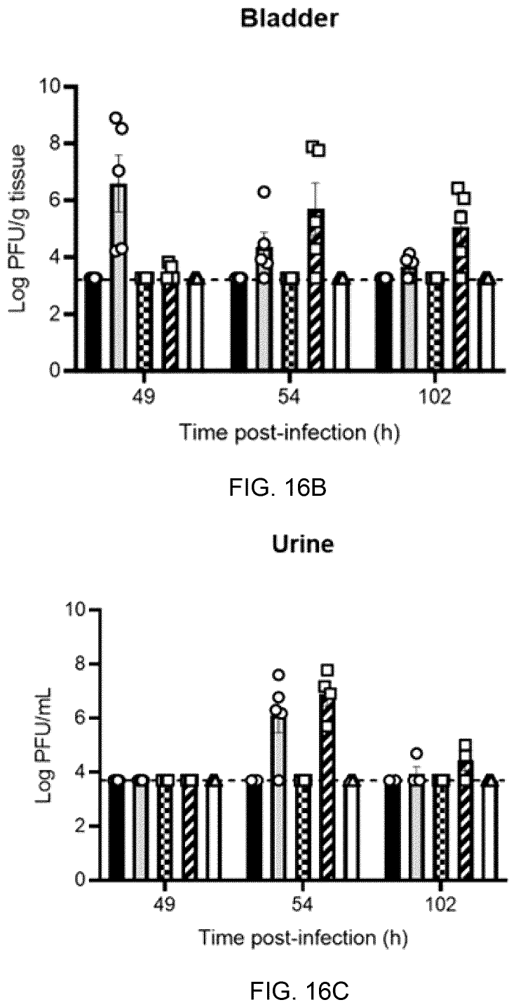

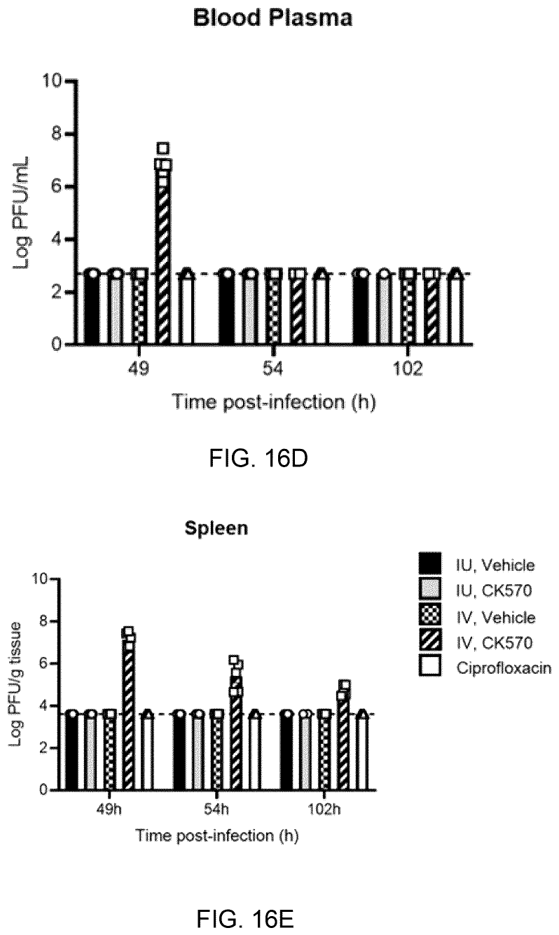

Importantly, intravenous (IV) administration of CK570, a systemic route of administration, also achieved significant reduction of bacterial burden. Significant decreases in enumerated CFU levels were observed in kidneys at all timepoints, in bladder at 54 hours p.i. and 102 hours p.i., and in urine at 102 hours p.i (FIGS. 16A-16C). Reduction in bacterial burden ranged from ˜2 log to ˜3 log. Ciprofloxacin treatment significantly reduced bacterial burden to the LOD in each tissue, across all timepoints assessed. Of note, ciprofloxacin is not a standard-of-care antibiotic for human uncomplicated UTI, and the use of ciprofloxacin in this experiment was intended solely as a positive control to confirm that that the model was executed as expected. In addition to enumeration of CFU, CK570 bacteriophages were also enumerated by serial dilution and plaquing of filtered tissue homogenates, filtered plasma, and urine on overlays of ATCC 700928. IU administration of bacteriophage resulted in detectable levels of CK570 in kidneys, bladder, and urine, especially at early timepoints. IV administration also resulted in detectable levels of LBPEC01 in blood and spleen at early timepoints, but signal dropped to at or near the limit of detection by 102 hours p.i. Despite the lowest levels of detectable bacteriophages at 102 hours p.i., there was still a significant cocktail efficacy, as evidenced by significant reduction in bacterial burden by both routes of administration. Taken together, IU or IV administration of CK570 results in sufficient delivery of the cocktail to the key organs to achieve efficacy.

Therefore, Both IU and IV administration were effective routes of administration to achieve reduction in bacterial burden, demonstrating that systemic delivery of CK570 is a viable clinical route of administration. Moreover, efficacy was achieved after a single dose of CK570 and maintained following four additional doses. Taken together, these data indicate that CK570 is highly efficacious and is capable of significantly reducing E. coli bacterial burden in a UTI model.

In another study, schematic of the design shown in FIG. 14B, similar results were obtained using CK618. The experimental layout is presented in Table. 21.

TABLE 21
Experimental design

| | | Day 0 Infection | 48 hours Post-infection Treatment | | | |
|---|---|---|---|---|---|---|
| | | Bacteria, Dose, | Test Article | ROA and | Dosing | Experimental |
| Group | # | ROA, & Volume | and Dose | Volume | Schedule | Endpoints |
| 1 | 20 | E. coli | TBS | IU 50 μL | 48 hours post- | 54 and 102 hours |
| | | (b3675) | | | infection | post-infection: |
| | | 1 × 10⁹ CFU | TBS | IV 100 μL | 56, 72, 80, and 96 | Bladder, blood, |
| | | IU | | | hours post-infection | and kidneys, |
| 2 | 20 | 50 μL | TBS | IU 50 μL | 48 hours post- | were collected |
| | | | | | infection | from n = 5 |
| | | | CK618 3.0 × | IV 100 μL | 56, 72, 80, and 96 | mice/timepoint |
| | | | 10¹¹ PFU/mL | | hours post-infection | |
| 3 | 20 | | CK618 3.0 × | IU 50 μL | 48 hours post- | |
| | | | 10¹¹ PFU/mL | | infection | |
| | | | CK618 3.0 × | IV 100 μL | 56, 72, 80, and 96 | |
| | | | 10¹¹ PFL/mL | | hours post-infection | |

CFU = colony forming unit: IU = intraurethral; IV = intravenous; PFU = plaque forming unit; ROA = route of administration; TBS = tris buffered saline. Cocktail dose is listed as composite potency.

### Results

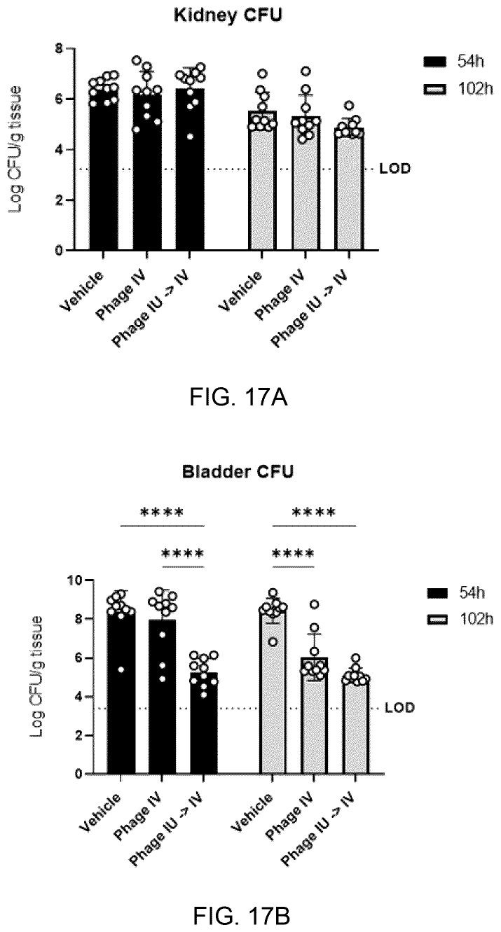

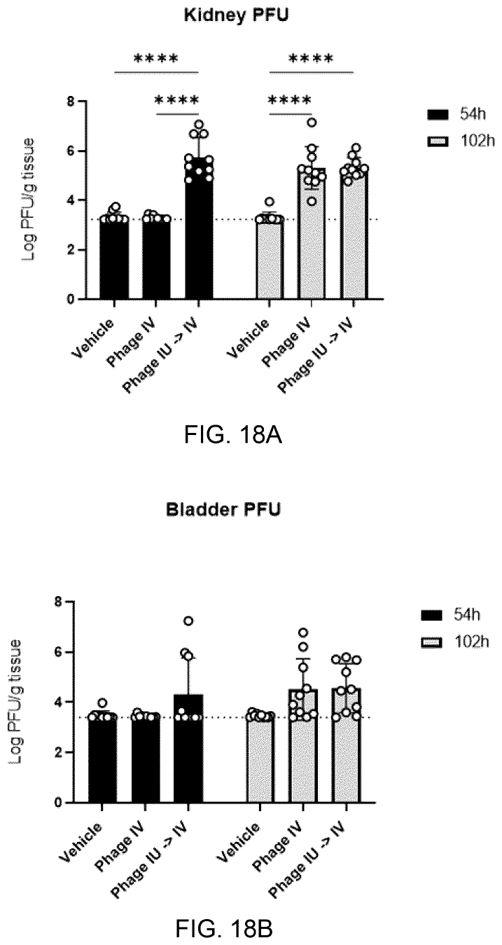

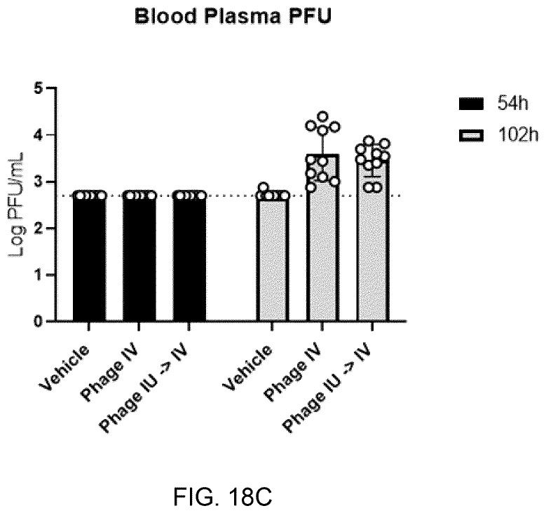

It was observed that in bladder, administration of both an IU dose and an IV dose of CK618 at 48 hours p.i. led to a significant, ˜3 log decrease in CFU at 54 hours relative to either the vehicle treatment or the IV dose alone (FIG. 17B). By contrast, at the 102 hour timepoint, both treatment groups that received 5 IV doses of CK618 showed a statistically significant, 2 to 3 log decrease in CFU relative to the vehicle-treated group, with no significant difference between the groups that did versus did not receive an initial IU dose of CK618. Therefore, an initial IU dose improved the efficacy of a single IV dose of CK618 but did not provide a boost in efficacy relative to five IV doses administered BID. Enumeration of the cocktail bacteriophage showed very few detectable bacteriophage at 54 hours in the kidneys of animals treated only with a single IV dose of CK618 at 48 hours p.i., whereas bacteriophage were clearly detectable in kidneys at the 102 hour timepoint, after 5 IV doses (FIG. 18A). By contrast, animals that received an IU dose at 48 hours p.i. prior to IV dosing had clearly detectable bacteriophage in kidneys at both timepoints. In bladder and blood plasma (FIGS. 18B-18C), the IU loading dose did not cause a significant change in biodistribution at either timepoint relative to IV dosing alone. Consistent with the efficacy data, these results suggest that an IU loading dose may improve the biodistribution of bacteriophage to the urinary tract relative to a single IV dose, but not relative to 5 BID IV doses.

TABLE 23
Cpf1 sequences

| | | ec STRAIN | |
|---|---|---|---|
| Name | SEQUENCE | COVERAGE % | TARGET |
| Ec_SC_Cpf1_4 | cattggtagccatgtttctttcctg (SEQ ID NO: 35) | 99.82% | UMP kinase |
| Ec_SC_Cpf1_5 | atactggaggagtcataagaattcg (SEQ ID NO: 36) | 99.82% | DNA gyrase subunit B |
| Ec_SC_Cpf1_7 | cggcgaccagtgccgtagtattgat (SEQ ID NO: 37) | 99.81% | 30s ribosomal protein |
| COMBINED | | 99.93% | |

TABLE 24
Example CRISPR system components

| | SEQ ID NO | Sequence |
|---|---|---|
| Cpf1 | 46 | agtatctatcaggaattcgtaaacaaatattccctgtcgaaaacgctgcgctttgaattaattccacaaggcaaaacgctggaaaacatca |
| | | aagcgcgtggccttatcctggatgacgaaaagcgtgcaaaagactacaagaaagctaaacaaatcatcgacaaatatcatcagtttttta |
| | | ttgaagaaattcttagctccgtgtgtatcagtgaagaccttcttcagaactatagcgatgtttacttcaagctgaagaaatcggacgatgat |
| | | aatctgcagaaagattttaagagtgccaaggatactatcaagaaacagattagtgagtacattaaggattccgagaagttcaaaaacctg |
| | | ttcaaccagaacctgatcgatgccaagaaagggcaggaatccgatctgatcctgtggcttaaacaatccaaagataacggcatcgaac |
| | | tgtttaaggcaaatagcgacatcacagatatcgatgaagcactggaaattatcaaatcgttcaaagggtggacaacctacttcaaaggat |
| | | ttcatgaaaatcgtaaaaacgtgtacagctcaaacgacatcccaacctctatcatttaccgcatcgttgacgataatttacccaaatttttgg |
| | | agaacaaagcgaaatatgaatctctgaaggataaggcgccggaagcgatcaattatgaacagatcaaaaaggacttggcggaggagt |
| | | tgacttttgatattgactataaaacgtcggaagtgaaccagcgcgtctttagcctggacgaagttttcgagattgcaaactttaataactact |
| | | taaatcaatccgggattactaagtttaatacgattattggcgggaaattcgttaacggtgaaaacaccaaacgtaaaggtatcaacgaata |
| | | cattaatctgtatagccagcaaatcaacgataaaacactcaaaaaatataagatgtccgtgctgttcaaacagattttaagtgatacggaa |
| | | agtaagtcatttgtgattgacaaactggaagatgactccgatgtagtgacgactatgcagtcattttatgaacagattgcggctttcaaaac |
| | | cgtcgaagagaagtcgatcaaagagacgctcagcctcttatttgatgatctcaaagcccagaagctggatctttcgaaaatctactttaaa |
| | | aacgataagtctttaaccgacctgtcccaacaggtgtttgatgactactccgtgattggtactgccgtgctggaatatattacccagcaaat |
| | | tgctccgaaaaacttagataacccaagtaagaaagaacaggaactgattgcgaaaaaaacggaaaaagcgaaatatctgagcctgga |
| | | aaccattaaactggccctggaagaatttaacaaacatcgtgatatcgacaaacaatgccgtttcgaagaaatcctggccaattttgccgc |
| | | cattccgatgattttcgatgaaatcgcacagaataaggacaatctggcgcagatctcaattaaataccagaatcagggtaagaaagatct |
| | | cctgcaggctagcgccgaagatgatgtgaaagcgatcaaagacctgctggaccaaactaataatctgctgcataaactgaagatctttc |
| | | acattagccagtcagaagacaaggcgaacattctcgataaagatgaacacttctacctggttttcgaagaatgctacttcgaattagcaaa |
| | | cattgtccccctctataataaaattcgcaattatattactcagaaaccctatagcgatgaaaaattcaaactgaatttcgaaaactccaccct |
| | | ggcgaacggttgggataagaataaagagccagataataccgccattctgtttatcaaagacgataaatattatttaggcgtcatgaacaa |
| | | gaaaaacaacaaaatctttgacgacaaagccatcaaggaaaataagggtgaaggttacaagaaaatcgtttataaactgctgcctggg |
| | | gcgaacaaaatgctgccgaaagtgttttttagcgccaagagcattaaattttataatccgagtgaagacatcctgcgcatccgtaatcattc |
| | | gacgcataccaaaaatggcagcccgcaaaaaggctatgaaaagtttgagttcaatattgaagattgtcgtaagtttatcgacttttacaaa |
| | | caatcgatcagcaaacatcccgaatggaaagactttggcttccgtttctccgacacccaacgttataactcgatcgatgagttctatcgcg |
| | | aggtcgaaaatcagggttataaactgacattcgaaaacatcagcgagtcatacattgactccgttgtaaatcagggcaaactgtacctctt |
| | | ccagatttataataaagatttcagcgcgtactcaaagggccgccccaatctccataccttatattggaaggcattgtttgatgagcgtaatc |
| | | tccaggatgtagtgtacaaactcaatggcgaggcggaactgttttatcgtaaacagagcattccaaagaaaattacccacccggcgaag |
| | | gaagcgattgccaacaaaaacaaggacaatccgaaaaaggaaagcgtgttcgagtacgacctgattaaagacaaacgctttacggaa |
| | | gataagtttttctttcactgcccgatcacgattaactttaaatcgtccggcgcaaacaaatttaacgacgaaattaacttgctgctgaaagag |
| | | aaagcgaacgatgtgcacattctgtcgatcgatcgtggtgaacgccatctggcgtattacacgctggtggatggcaagggtaacattat |
| | | caagcaggataccttcaatatcatcggtaatgatcgcatgaaaacgaattatcacgacaaactggctgctatcgagaaagatcgtgatag |
| | | cgctcgcaaagactggaaaaaaatcaacaacatcaaggaaatgaaagaaggttatctttctcaggtagtacacgaaatcgccaaattag |
| | | tcattgaatataatgctattgtagtgtttgaagatctgaactttggtttcaaacgcggtcgcttcaaggtcgaaaaacaggtttaccagaaatt |
| | | agagaaaatgttgattgagaaactgaactatctggtcttcaaggacaatgagtttgataagacaggtggcgttctgcgtgcgtatcagctg |
| | | accgccccgttcgaaaccttcaagaaaatgggtaaacagacgggtattatctattacgtgccggcaggcttcaccagcaagatttgccc |
| | | ggtcacgggcttcgtcaaccaactgtacccaaagtatgaaagtgtatccaagagccaggagttctttagcaagtttgataaaatctgctac |
| | | aacctggataagggctatttcgagttcagcttcgactacaaaaacttcggcgacaaagccgcgaaaggcaagtggacgattgcctcctt |
| | | cggcagccgcctgattaactttcgcaactcggacaaaaatcataattgggatacccgcgaagtgtatcctacgaaagagctggagaaa |
| | | ctgctcaaagactactcgatcgaatatggtcacggtgagtgtattaaagccgcaatttgcggtgaaagcgataaaaagtttttcgccaaa |
| | | ctcaccagcgtattaaatactatccttcagatgcgcaattccaagacaggtaccgaactcgattatctgatctcgccggtggggatgtga |
| | | acgggaacttctttgacagccgccaggcgccgaaaaatatgccacaagacgcagatgcaaatggagcttatcacattggtctgaaagg |
| | | acttatgctcctgggccgtattaaaaataaccaggagggtaagaaacttaatctggttattaaaaacgaagaatattttgagttcgttcaga |
| | | accgtaacaactaa |
| Cas3 | 47 | ATGGACGCGGAGGCTAGCGATACTCACTTTTTTGCTCACTCCACCTTAAAGGCAG |
| | | ATCGCAGCGATTGGCAGCCTCTGGTCGAGCATCTACAGGCTGTTGCCCGTTTGGC |
| | | AGGAGAGAAGGCTGCCTTCTTCGGCGGCGGTGAATTAGCTGCTCTTGCTGGTCTG |
| | | TTGCATGACTTGGGTAAATACACTGACGAGTTTCAGCGGCGTATTGCGGGTGATG |
| | | CCATCCGTGTCGATCACTCTACTCGCGGGGCCATACTGGCGGTAGAACGCTATGG |
| | | CGCGCTAGGTCAATTGCTAGCCTACGGCATCGCTGGCCACCATGCCGGGTTGGCC |
| | | AATGGCCGCGAGGCTGGTGAGCGAACTGCCTTGGTCGACCGCCTGAAAGGGGTT |
| | | GGGCTGCCACGGTTATTGGAGGGGTGGTGCGTGGAAATCGTGCTACCCGAGCGC |
| | | CTTCAACCACCGCCACTAAAAGCGCGCCTGGAAAGAGGTTTCTTTCAGTTGGCCT |
| | | TTCTTGGCCGGATGCTCTTTTCCTGCTTGGTTGATGCGGATTATCTAGATACCGAA |
| | | GCCTTCTACCACCGCGTCGAAGGACGGCGCTCCCTTCGCGAGCAAGCGCGGCCG |
| | | ACCTTGGCCGAGTTACGCGCAGCCCTTGATCGGCATCTGACTGAGTTCAAGGGAG |
| | | ATACGCCGGTCAACCGCGTTCGCGGGGAGATATTGGCCGGCGTGCGCGGCAAGG |
| | | CGAGCGAACTTCCCGGGCTGTTTTCTCTCACAGTGCCCACAGGAGGCGGCAAGAC |
| | | CCTGGCCTCTCTGGCTTTCGCCCTGGATCACGCTCTAGCTCATGGGCTGCGCCGG |
| | | GTGATCTACGTGATTCCCTTCACTAGCATCGTCGAGCAGAACGCTGCGGTATTCC |
| | | GTCGTGCACTCGGGGCCTTAGGCGAAGAGGCGGTGCTGGAGCATCACAGCGCCT |
| | | TCGTTGATGACCGCCGGCAGAGCCTGGAGGCCAAGAAGAAACTGAACCTAGCGA |
| | | TGGAGAACTGGGACGCGCCTATCGTGGTGACCACTGCAGTGCAGTTCTTCGAAAG |
| | | CCTGTTTGCCGACCGTCCAGCCCAGTGCCGCAAGCTACACAACATCGCCGGCAGC |
| | | GTGGTGATTCTTGACGAGGCACAGACCCTACCGCTCAAGCTGTTGCGGCCCTGCG |
| | | TTGCCGCCCTTGATGAACTGGCGCTCAACTACCGTTGTAGCCCAGTTCTCTGTACT |
| | | GCCACGCAGCCAGCGCTTCAATCGCCGGATTTCATCGGTGGGCTGCAGGACGTAC |
| | | GTGAGCTGGCGCCCGAGCCGCAGCGGCTGTTCCGGGAGTTGGTGCGGGTACGAA |
| | | TACGGACATTGGGCCCGCTCGAAGATGCGGCCTTGACTGAGCAGATCGCCAGGC |
| | | GTGAACAAGTGCTGTGCATCGTCAACAATCGACGCCAGGCCCGTGCGCTCTATGA |
| | | GTCGCTTGCCGAGTTGCCCGGTGCCCGCCATCTCACCACCCTGATGTGCGCCAAG |
| | | CACCGTAGCAGCGTGCTGGCCGAGGTGCGCCAGATGCTCAAAAAGGGGGAGCCC |
| | | TGTCGCCTGGTGGCCACCTCGCTGATCGAGGCCGGTGTGGATGTGGATTTTCCCG |
| | | TGGTACTGCGTGCCGAGGCTGGATTGGATTCCATCGCCCAGGCCGCGGGACGCTG |
| | | CAATCGCGAAGGCAAGCGGCCGCTGGCCGAAAGCGAGGTGCTGGTGTTCGCCGC |
| | | GGCCAATTCTGACTGGGCGCCACCCGAGGAACTCAAGCAGTTCGCCCAGGCCGC |
| | | CCGCGAAGTGATGCGCCTGCACCCGGATGATTGCCTGTCCATGGCGGCCATCGAG |
| | | CGGTATTTTCGCATACTGTACTGGCAGAAGGGCGCGGAGGAGTTGGATGCGGGT |
| | | AACCTGCTCGGCCTGATTGAGAGAGGCCGGCTCGATGGCCTGCCCTACGAGACTT |
| | | TGGCCACCAAGTTCCGCATGATCGACAGCCTTCAACTGCCGGTGATCATCCCATT |
| | | TGATGACGAGGCCAGAGCAGCCCTGCGCGAGCTGGAGTTCGCCGACGGCTGCGC |
| | | CGCCATCGCCCGTCGCCTGCAGCCATATCTGGTGCAGATGCCACGCAAGGGTTAT |
| | | CAGGCATTGCGGGAAGCCGGTGCGATCCAGGCGGCGGCAGGTACGCGTTATGGT |
| | | GAGCAGTTTATGGCGTTGGTCAACCCTGATCTGTATCACCACCAATTCGGGTTGC |
| | | ACTGGGATAATCCGGCCTTTGTCAGCAGCGAGCGGCTATGTTGGTAG |
| Cas5c | 48 | ATGGCCTACGGAATTCGCTTAATGGTCTGGGGCGAGCGTGCCTGCTTCACCCGCC |
| | | CGGAAATGAAGGTGGAACGCGTCTCTTACGATGCGATCACGCCGTCCGCCGCGC |
| | | GCGGCATTCTCGAGGCTATCCACTGGAAGCCGGCGATTCGCTGGGTGGTGGATCG |
| | | CATTCAAGTGCTTAAGCCGATCCGCTTCGAATCCATCCGGCGCAACGAGGTCGGC |
| | | GGCAAGCTGTCCGCTGTCAGCGTCGGTAAGGCAATGAAGGCCGGGCGTACTAAT |
| | | GGTCTGGTGAATCTGGTCGAGGAGGATCGCCAGCAGCGCGCGACTACTCTGCTGC |
| | | GCGATGTCTCCTATGTCATCGAGGCGCATTTCGAGATGACTGACAGGGCTGGCGC |
| | | CGACGATACGGTGGGCAAGCATCTGGATATCTTCAACCGTCGCGCACGGAAGGG |
| | | GCAGTGCTTCCATACACCCTGCCTAGGCGTGCGCGAGTTTCCGGCCAGTTTTCGG |
| | | TTGCTGGAAGAGGGCAGTGCCGAGCCTGAAGTCGATGCCTTTCTGCGCGGCGAG |
| | | CGTGATCTGGGCTGGATGCTGCATGACATTGACTTCGCCGATGGCATGACCCCGC |
| | | ACTTCTTCCGTGCCCTGATGCGCGATGGGCTGATCGAGGTGCCGGCCTTCAGGGC |
| | | GGCAGAGGACAAGGCATGA |
| Cas8c | 49 | ATGATCCTTTCGGCCCTCAATGACTATTATCAGCGACTGCTGGAGCGGGGTGAAG |
| | | CGAATATCTCACCCTTCGGCTACAGCCAAGAAAAGATCAGTTACGCCCTGCTGCT |
| | | GTCCGCACAAGGAGAGTTGCTGGACGTGCAGGACATTCGCTTGCTCTCTGGCAAG |
| | | AAGCCTCAACCCAGGCTTATGAGTGTGCCGCAGCCGGAGAAGCGCACCTCGGGC |
| | | ATCAAGTCCAACGTACTGTGGGACAAGACCAGCTATGTGCTGGGTGTTAGTGCCA |
| | | AGGGCGGAGAGCGTACTCAGCAGGAGCACGAGTCCTTCAAGACGCTGCACCGGC |
| | | AGATCTTGGTTGGGGAAGGCGACCCCGGTCTGCAGGCCTTGCTCCAGTTCCTCGA |
| | | CTGTTGGCAGCCGGAGCAGTTCAAGCCCCCGCTGTTCAGCGAAGCAATGCTCGAC |
| | | AGCAACTTAGTGTTCCGCCTAGACGGCCAACAACGCTATCTGCACGAGACTCCGG |
| | | CGGCCCTGGCGTTGCGTACCCGGCTGTTGGCCGACGGCGACAGCCGCGAGGGGC |
| | | TGTGCCTAGTCTGCGGCCAACGTCAGCCGTTGGCGCGCCTGCATCCAGCGGTCAA |
| | | GGGCGTCAATGGTGCCCAGAGTTCGGGGGCTTCCATCGTCTCCTTCAACCTCGAC |
| | | GCTTTTTCCTCCTACGGCAAGAGCCAGGGGGAAAATGCTCCGGTCTCCGAACAGG |
| | | CCGCCTTTGCCTACACCACGGTGCTCAACCATTTGTTGCGTCGCGACGAGCACAA |
| | | CCGCCAGCGCCTGCAGATTGGCGACGCGAGTGTGGTGTTCTGGGCGCAGGCGGA |
| | | TACTCCTGCTCAGGTGGCCGCCGCCGAGTCGACCTTCTGGAACCTGCTGGAGCCA |
| | | CCCGCAGATGATGGTCAGGAAGCGGAAAAGCTGCGCGGCGTGCTGGATGCTGTG |
| | | GCCACGGGGCGGCCCTTGCATGAGCTCGACTCGCTAATGGAGGAAGGTACCCGC |
| | | ATTTTTGTGTTAGGGCTGGCGCCCAATACCTCGCGACTGTCCATTCGGTTCTGGGC |
| | | AGTCGATAGCCTTGCGGTATTCACCCAGCATCTGGCCGAGCATTTCCGGGATATG |
| | | CACCTTGAGCCTCTGCCCTGGAAGACGGAGCCGGCCATCTGGCGCTTGCTCTATG |
| | | CTACCGCGCCCAGTCGTGACGGCAGAGCCAAGACCGAAGACGTACTCCCACAAC |
| | | TGGCCGGTGAAATGACCCGCGCCATCCTGACCGGCAGCCGCTATCCGCGCAGTTT |
| | | GCTAGCCAACCTGATCATGCGCATGCGTGCCGACGGCGACGTCTCTGGCATACGC |
| | | GTCGCGCTGTGCAAGGCCGTGCTCGCTCGCGAGGCACGCCTGAGCGGCAAAATT |
| | | CACCAAGAGGAGCTACCTATGAGTCTCGACAAGGACGCCAGCAACCCCGGCTAT |
| | | CGCTTGGGGAGGCTGTTCGCCGTGTTGGAAGGCGCCCAGCGCGCAGCCCTGGGC |
| | | GACAGGGTCAATGCCACTATCCGTGACCGCTACTACGGTGCCGCGTCCAGCACGC |
| | | CAGCCACGGTTTTCCCGATACTGCTGCGCAACACACAAAACCACTTGGCCAAGCT |
| | | GCGCAAGGAGAAGCCCGGACTAGCAGTGAACCTAGAGCGCGATATAGGCGAAAT |
| | | CATTGACGGTATGCAGAGCCAATTCCCGCGTTGCCTGCGCCTGGAGGACCAGGG |
| | | ACGCTTTGCTATTGGTTACTACCAACAGGCCCAGGCCCGTTTCAACCGTGGCCCC |
| | | GATTCCGTCGAGTAA |
| Cas7c | 50 | ATGACCGCCATCTCCAACCGCTACGAGTTCGTTTACCTCTTTGATGTCAGCAATG |
| | | GCAATCCCAATGGCGACCCGGATGCTGGCAACATGCCGCGTCTCGATCCGGAAA |
| | | CCAACCAGGGGTTGGTCACTGACGTTTGCCTCAAGCGCAAGATCCGCAACTACGT |
| | | CAGCCTGGAGCAGGAAAGTGCCCCCGGCTATGCCATCTATATGCAGGAAAAATC |
| | | CGTGCTGAATAACCAGCACAAACAGGCCTACGAGGCGCTCGGTATCGAGTCAGA |
| | | GGCAAAGAAACTGCCCAAGGACGAAGCCAAGGCGCGCGAACTGACCTCTTGGAT |
| | | GTGCAAGAACTTCTTCGATGTGCGTGCTTTCGGGGCGGTGATGACCACCGAGATT |
| | | AATGCCGGCCAGGTGCGTGGACCGATCCAACTGGCATTCGCCACGTCTATCGACC |
| | | CGGTATTGCCTATGGAGGTATCCATCACCCGCATGGCGGTGACTAACGAAAAGG |
| | | ATTTGGAGAAGGAACGCACCATGGGACGCAAGCACATCGTGCCTTACGGCTTGT |
| | | ACCGCGCCCATGGTTTCATCTCTGCCAAGTTGGCCGAGCGAACCGGCTTTTCCGA |
| | | CGACGACTTGGAACTGCTATGGCGCGCTTTGGCCAATATGTTCGAACACGACCGC |
| | | TCGGCGGCACGTGGCGAGATGGCAGCGCGCAAGTTGATCGTCTTCAAGCATGAG |
| | | CATGCCATGGGCAATGCACCCGCCCATGTGCTGTTCGGCAGCGTTAAGGTCGAGC |
| | | GAGTCGAGGGGGACGCAGTTACACCAGCACGCGGTTTCCAGGATTACCGTGTCA |
| | | GCATCGATGCGGAAGCTCTGCCTCAGGGCGTGAGCGTGCGCGAGTACCTCTAG |
| Cas3 | 51 | MDAEASDTHFFAHSTLKADRSDWQPLVEHLQAVARLAGEKAAFFGGGELAALAGL |
| | | LHDLGKYTDEFQRRIAGDAIRVDHSTRGAILAVERYGALGQLLAYGIAGHHAGLAN |
| | | GREAGERTALVDRLKGVGLPRLLEGWCVEIVLPERLQPPPLKARLERGFFQLAFLGR |
| | | MLFSCLVDADYLDTEAFYHRVEGRRSLREQARPTLAELRAALDRHLTEFKGDTPVN |
| | | RVRGEILAGVRGKASELPGLFSLTVPTGGGKTLASLAFALDHALAHGLRRVIYVIPFT |
| | | SIVEQNAAVFRRALGALGEEAVLEHHSAFVDDRRQSLEAKKKLNLAMENWDAPIVV |
| | | TTAVQFFESLFADRPAQCRKLHNIAGSVVILDEAQTLPLKLLRPCVAALDELALNYRC |
| | | SPVLCTATQPALQSPDFIGGLQDVRELAPEPQRLFRELVRVRIRTLGPLEDAALTEQIA |
| | | RREQVLCIVNNRRQARALYESLAELPGARHLTTLMCAKHRSSVLAEVRQMLKKGEP |
| | | CRLVATSLIEAGVDVDFPVVLRAEAGLDSIAQAAGRCNREGKRPLAESEVLVFAAAN |
| | | SDWAPPEELKQFAQAAREVMRLHPDDCLSMAAIERYFRILYWQKGAEELDAGNLLG |
| | | LIERGRLDGLPYETLATKFRMIDSLQLPVIIPFDDEARAALRELEFADGCAAIARRLQP |
| | | YLVQMPRKGYQALREAGAIQAAAGTRYGEQFMALVNPDLYHHQFGLHWDNPAFVS |
| | | SERLCW* |
| Cas5c | 52 | MAYGIRLMVWGERACFTRPEMKVERVSYDAITPSAARGILEAIHWKPAIRWVVDRIQ |
| | | VLKPIRFESIRRNEVGGKLSAVSVGKAMKAGRINGLVNLVEEDRQQRATTLLRDVS |
| | | YVIEAHFEMTDRAGADDTVGKHLDIFNRRARKGQCFHTPCLGVREFPASFRLLEEGS |
| | | AEPEVDAFLRGERDLGWMLHDIDFADGMTPHFFRALMRDGLIEVPAFRAAEDKA* |
| Cas8c | 53 | MILSALNDYYQRLLERGEANISPFGYSQEKISYALLLSAQGELLDVQDIRLLSGKKPQ |
| | | PRLMSVPQPEKRTSGIKSNVLWDKTSYVLGVSAKGGERTQQEHESFKTLHRQILVGE |
| | | GDPGLQALLQFLDCWQPEQFKPPLFSEAMLDSNLVFRLDGQQRYLHETPAALALRTR |
| | | LLADGDSREGLCLVCGQRQPLARLHPAVKGVNGAQSSGASIVSFNLDAFSSYGKSQG |
| | | ENAPVSEQAAFAYTTVLNHLLRRDEHNRQRLQIGDASVVFWAQADTPAQVAAAEST |
| | | FWNLLEPPADDGQEAEKLRGVLDAVATGRPLHELDSLMEEGTRIFVLGLAPNTSRLSI |
| | | RFWAVDSLAVFTQHLAEHFRDMHLEPLPWKTEPAIWRLLYATAPSRDGRAKTEDVL |
| | | PQLAGEMTRAILTGSRYPRSLLANLIMRMRADGDVSGIRVALCKAVLAREARLSGKI |
| | | HQEELPMSLDKDASNPGYRLGRLFAVLEGAQRAALGDRVNATIRDRYYGAASSTPA |
| | | TVFPILLRNTQNHLAKLRKEKPGLAVNLERDIGEIIDGMQSQFPRCLRLEDQGRFAIG |
| | | YYQQAQARFNRGPDSVE* |
| Cas7c | 54 | MTAISNRYEFVYLFDVSNGNPNGDPDAGNMPRLDPETNQGLVTDVCLKRKIRNYVS |
| | | LEQESAPGYAIYMQEKSVLNNQHKQAYEALGIESEAKKLPKDEAKARELTSWMCKN |
| | | FFDVRAFGAVMTTEINAGQVRGPIQLAFATSIDPVLPMEVSITRMAVTNEKDLEKERT |
| | | MGRKHIVPYGLYRAHGFISAKLAERTGFSDDDLELLWRALANMFEHDRSAARGEMA |
| | | ARKLIVFKHEHAMGNAPAHVLFGSVKVERVEGDAVTPARGFQDYRVSIDAEALPQG |
| | | VSVREYL* |

### Example 14: A Bacteriophage Cocktail

An example drug product is a sterile, aqueous cocktail of 6 bacteriophages, three of which have been engineered to include a CRISPR/Cas3 construct targeting highly conserved regions of the E. coli genome (p004ke009, p00c0e030, and p00exe014) and three of which are wild-type (p00jc, p00ke, and p5516), in a vehicle. Each vial nominally contains 6.34 mL of solution at an aggregate titer of 1.67×10¹¹ PFU/mL; a dose of 6.0 mL would deliver 1×10¹² PFU. The routes of administration are oral, intravesical, and intravenous. Additional characteristics are listed in Table 25.

TABLE 25
Drug characteristics

| Particulate Matter | Particulate matter in injections by | ≥10 μm: Average ≤6000 particles/vial | |
|---|---|---|---|
| | USP/NF<788>/DUR-QCT-1762-TM1.3 | ≥25 μm: Average ≤600 particles/vial | |
| Identity | Whole-genome sequencing/DUR-QCT- | Reads detected that uniquely map to each | |
| | 1546-TM; Data analysis: STM-QCB-006 | of six bacteriophage reference genomes | |
| Identity | Whole-genome sequencing/DUR-QCT- | ≤1% single nucleotide polymorphisms in | |
| | 1546-TM; Data analysis: STM-QCB-006 | reads mapped to concatenated reference | |
| | | genomes | |
| Potency | Enumeration by plaquing on strain | 1 × 10⁹-1 × 10¹² | PFU/mL |
| | b1921/DUR-QCT-1743-TM | | |
| Potency | Enumeration by plaquing on strain | 1 × 10⁹-1 × 10¹² | PFU/mL |
| | b1890/DUR-QCT-1743-TM | | |
| Potency | Enumeration by plaquing on strain | 1 × 10⁹-1 × 10¹² | PFU/mL |
| | b2297/DUR-QCT-1743-TM | | |
| Microbial | Sterility by USP/NF<71>/LM 141 | No growth detected | |
| contaminants | | | |
| Microbial | Endotoxin by USP/NF<85>/DUR-QCT- | ≤50.00 | EU/mL |

| | |
|---|---|
| contaminants | 1771-TM |

### Example 15: Host Range of Different Cocktails

The host range of three different cocktails were tested on a clinical validation panel of 304 strains, of which ninety-three were classified as multi-drug resistant. The cocktails are LBP-EC01 v.1, CK479 and CK507. CK479 and CK507 are surrogate cocktails for LBP-EC01 v.2. The components of the cocktails are listed in Table 1B. The results are depicted in Table 26.

TABLE 26
Host range of different cocktails

| Metric | LBP-EC01 v.1 | CK479 | CK507 |
|---|---|---|---|
| % host range | 56.4 | 73.0 | 68.3 |
| % optical density <0.4 | 32.9 | 52.3 | 44.9 |
| % host-range in multi-drug | 47.0 | 67.8 | 59.1 |
| resistant strains | | | |

### Example 16: LBP-EC01 v.2 Shows Good Depth of Kill and Suppression of Regrowth by CFU Reduction Assay

The CFU reduction assay measures the ability of an individual bacteriophage or bacteriophage cocktail to deplete the bacteria present in a culture as well as to assess for the durability of this reduction. Efficacy is correlated with the magnitude of CFU depletion compared to the cells-only control and is said to have high durability if this depletion is maintained or remains significant over time, for example out to 24 hours post exposure. A subset panel of 31 E. coli isolates was defined from a panel of 300 clinically relevant isolates. These isolates were challenged with the surrogate cocktail CK570 at a total titer of 1×106 PFU/mL. Maximum log CFU reductions compared to control were assessed at 0, 4, 8, and 24 hours after the initial exposure.

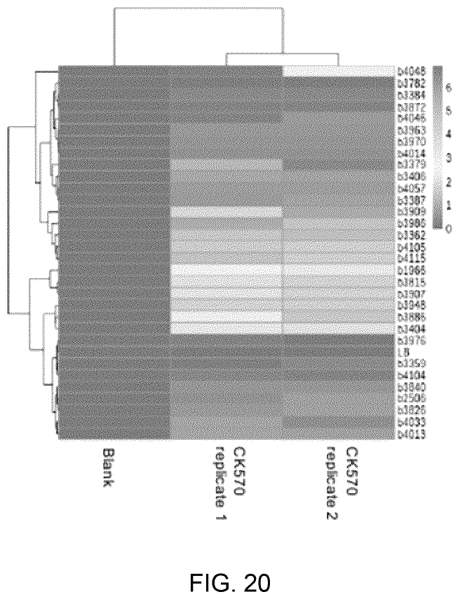

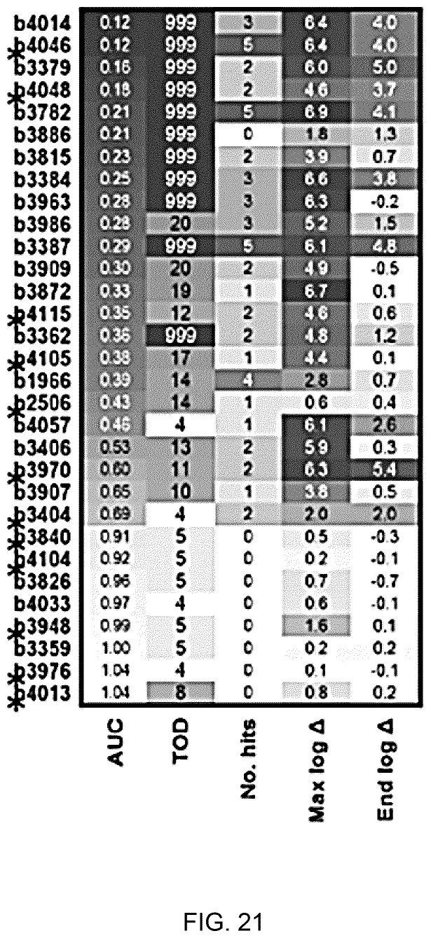

The heatmap in FIG. 20 shows larger max log CFU reductions in redder hues and smaller log CFU reductions in pale blues. Strains that were unaffected by bacteriophage treatment are in dark blue. In the leftmost column (“Blank”), the cells-only control is both the control and the test sample, so all values are 0. The center and rightmost column show two replicates of bacteriophage treatment. Data from the CFU reduction assay was also compared with AUC data from the liquid host range assay (FIG. 21). In general, bacteriophage treatment caused significant CFU reduction. Additionally, a subset of strains were depleted below the limit of detection (LOD) and even at 24 hours maintained this depletion (demonstrating good durability of bacteriophage kill). Specifically, 21/31 strains in the representative panel had CFU reductions of greater than 2-log at one of the timepoints, with 10/31 strains maintaining at least a 2-log suppression at the 24-hour timepoint.

Names on the left indicate bacterial strains; strains marked with an asterisk (*) are MDR strains. AUC and time to OD (TOD) columns are derived from liquid host range as described previously. TOD values of 999 indicate that the strain never rebounded to an OD600 of 0.4; other values indicate the time in hours at which the strain rebounded to an OD600 of 0.4. No. hits indicates the number of bacteriophages in the cocktail that target the strain listed on the left. Max log Δ is the largest log CFU reduction observed for the indicated strain at any timepoint. End log Δ is the log CFU reduction observed at the 24-hour timepoint for the indicated strain. Note that AUC values <0.65 (which we consider to represent bacteriophage targeting in our liquid host range assay) are generally predictive of a high max log Δ in the CFU reduction assay, and high TOD values from the liquid host range assay correlate well with high end log Δ values in the CFU reduction assay. This data validates the use of the liquid host range assay to determine whether and how strongly the bacteriophage cocktail targets a given strain.

### Example 17: Concomitant Treatment with Bacteriophage and Antibiotics

Antibiotics typically used to treat uncomplicated UTI were tested for host range in combination with and alongside LBP-EC01 v.2, as described in Table 1B, against two panels of contemporaneous (collected from 2017 to 2020) E. coli isolates from North America, Europe, Latin America, and Asia. The first assay used a panel of 88 contemporaneous isolates. Twenty-one of those strains are classified as MDR, with resistance to 3 or more antibiotic classes. Seven different antibiotics were tested against this panel (ceftriaxone, cephalexin, trimethoprim/sulfamethoxazole [TMP/SMX; also called cotrimoxazole], fosfomycin, and nitrofurantoin) and two antibiotics often used to treat complicated UTI (cefdinir and ciprofloxacin). The second assay used the panel made up of a 300-strain clinical isolate panel with an additional 4 strains of interest. Ninety-three of those isolates are classified as MDR. Four of the same antibiotics tested against the 88-isolate panel (ciprofloxacin, cotrimoxazole (TMP/SMX), cephalexin, and fosfomycin) were also tested against this larger panel.

Antibiotic concentrations tested were determined by using CLSI or EUCAST breakpoints at concentrations that kill susceptible strains but allow intermediate or resistant strains to grow in the standard AST microdilution assay (Table 27). LBP-EC01 v.2 was combined with bacteria resulting in a MO of 10 for each bacteriophage in a sample. In the case of the 88-isolate panel, results were analyzed on the complete panel as well as the subset of isolates classified as multidrug-resistant (MDR) (n=21) and beta-lactam-resistant (n=31). In the case of the 304-isolate panel, results were analyzed on the complete panel as well as the subset of isolates classified as MDR (n=93) and beta-lactam-resistant (n=91).

TABLE 27

| Antibiotic or | | Concentration | | Breakpoint |
|---|---|---|---|---|
| Phage Cocktail | Class | in Assay | | Reference |
| Cefdinir | 3^{rd} gen cephalosporin | 1 | .tg/ml | CLSI M100 Table 2A |
| Ceftriaxone | 3^{rd} gen cephalosporin | 1 | .tg/ml | CLSI M100 Table 2A |
| Cephalexin | 1^{st} gen cephalosporin | 16 | .tg/ml | EUCAST Breakpoint Table |
| | | | | v 11.0 |
| Ciprofloxacin | Fluoroquinolone | 0.25 | .tg/ml | CLSI M100 Table 2A |
| Cotrimoxazole | Folate pathway | 2 | .tg/ml | CLSI M100 Table 2A |
| (TMP/SMX) | | | | |
| Fosfomycin | Fosfomycin | 8 | .tg/ml | EUCAST Breakpoint Table |
| | | | | v 11.0 (uncomplicated UTI |
| | | | | only) |
| Fosfomycin | Fosfomycin | 64 | .tg/ml | CLSI M100 Table 2A |
| Nitrofurantoin | Nitrofurans | 32 | .tg/ml | CLSI M100 Table 2A |

| | | | |
|---|---|---|---|
| LBP-EC01 | Bacteriophage Cocktail | 6 × 10⁶ PFU/ml total, | Not applicable |
| | | 1 × 10⁶ PFU/ml per | |
| | | bacteriophage | |

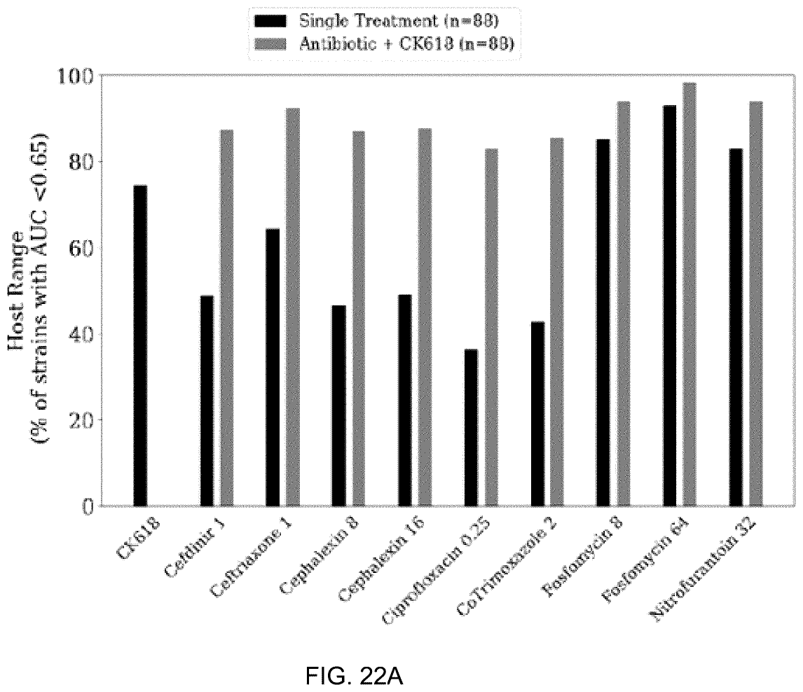

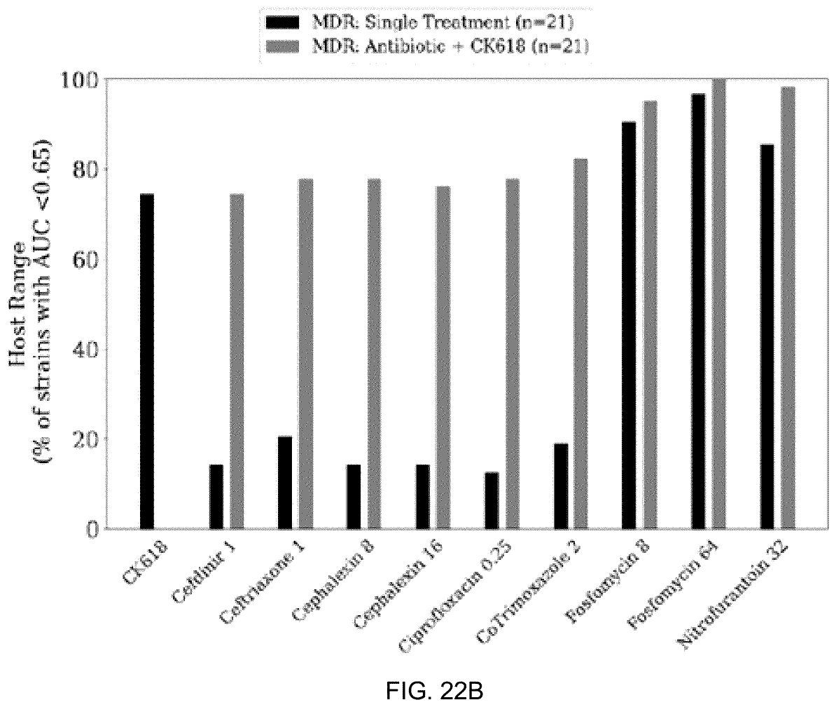

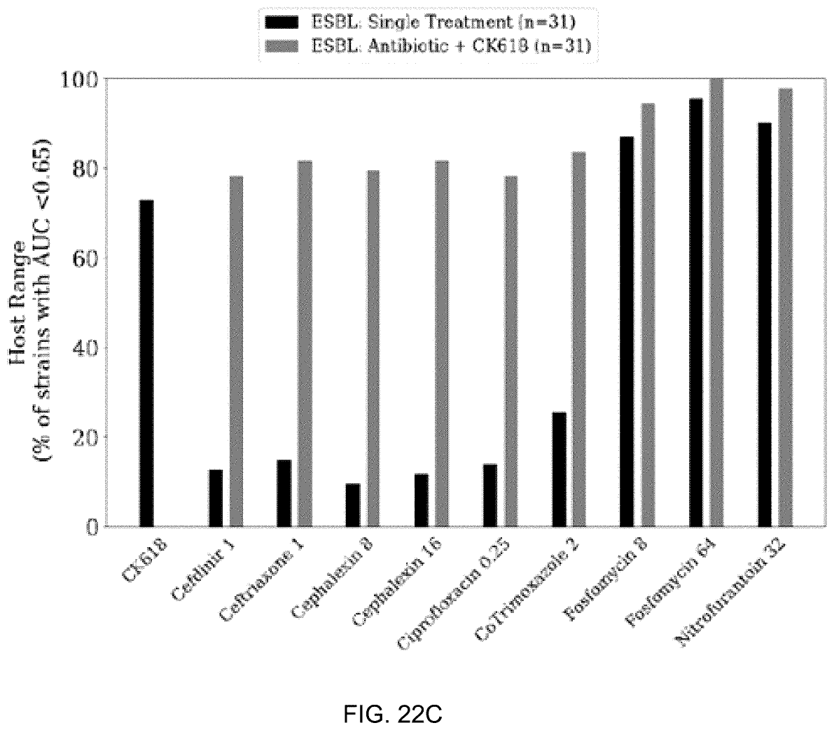

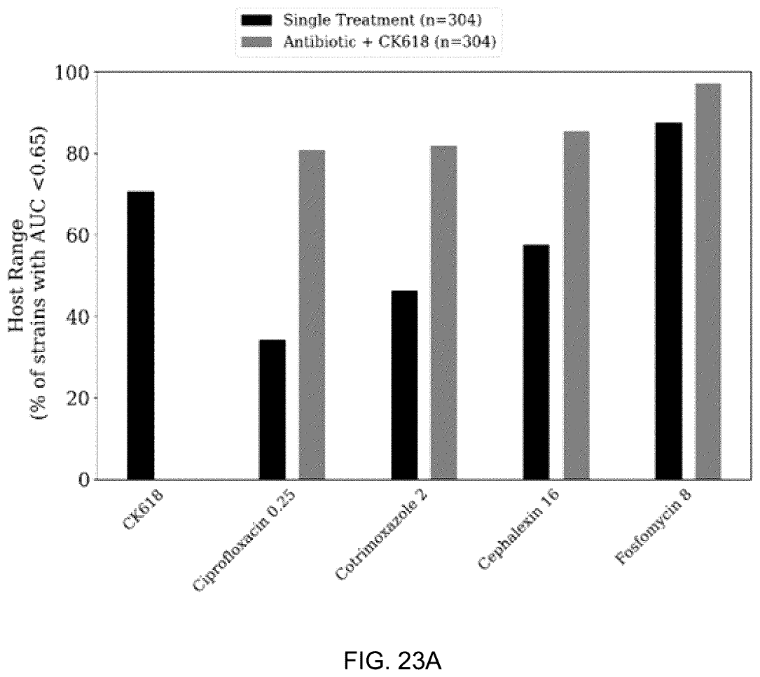

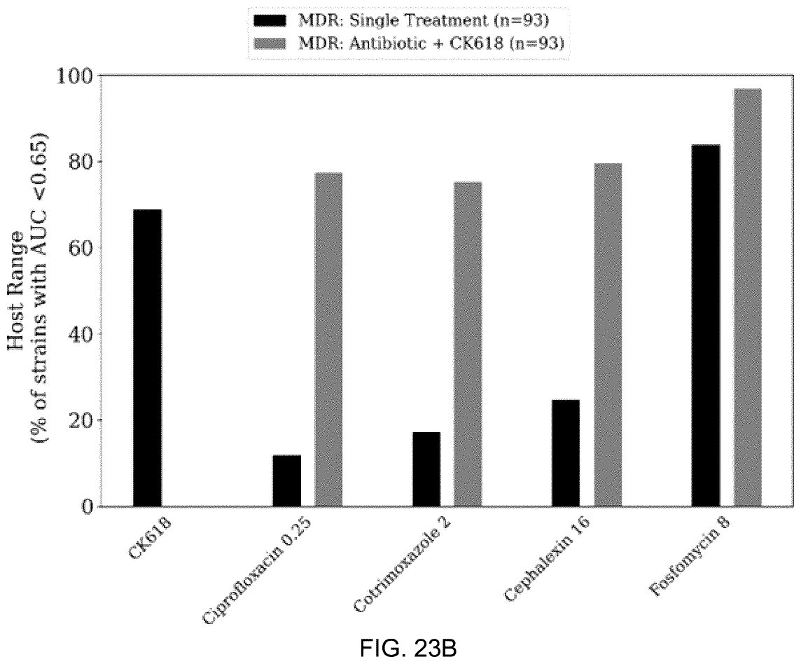

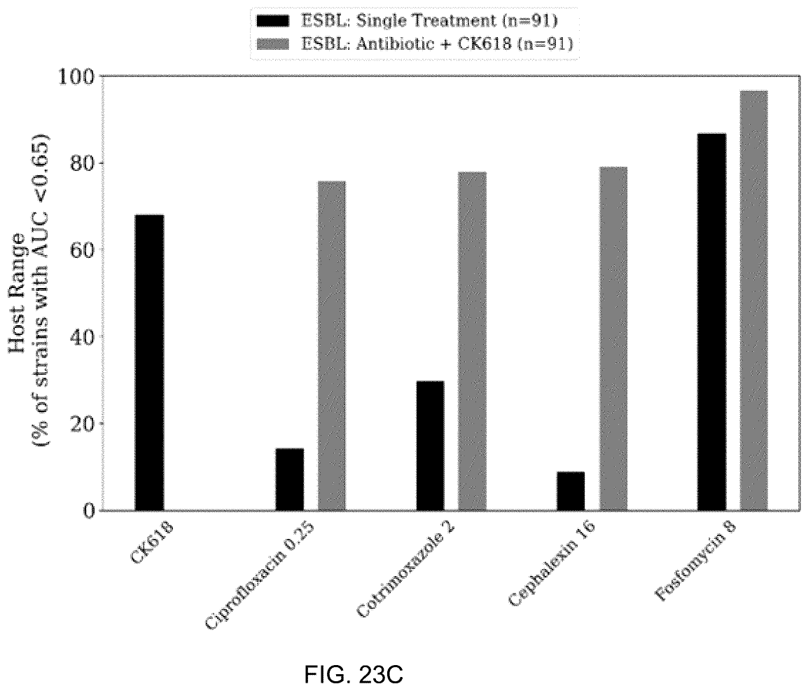

LBP-EC01 v.2 outperformed most of the antibiotics commonly used to treat uUTI that were tested, including first and third generation cephalosporins, ciprofloxacin, and TMP/SMX on all strains (FIG. 22, FIG. 23, Table 27, and Table 28), under these experimental conditions. Combination therapy was more effective in all cases compared to antibiotic treatment alone. In the MDR and beta-lactam-resistant subsets of strains, combination therapy with the cephalosporins, ciprofloxacin, or cotrimoxazole (TMP/SMX) performs similarly to or slightly better than the bacteriophage cocktail alone. Fosfomycin and nitrofurantoin had strong activity across all strains and were modestly improved by the addition of LBP-EC01 v.2. For MDR and beta-lactam-resistant strains, the overall host range of cephalosporins, ciprofloxacin, and cotrimoxazole (TMP/SMX) was reduced while LBP-EC01 v.2, nitrofurantoin, and fosfomycin performed similarly regardless of antibiotic resistance status across the isolates tested (FIG. 22, FIG. 23, Table 28, and Table 29).

TABLE 28
Host Range of LBP-EC01 v.2 and Antibiotics Alone and in Combination on 88-Isolate Panel

| | Host Range % on all | | Host Range % on | | Host Range % on Beta-lactam- | |
|---|---|---|---|---|---|---|
| | Strains (n = 88) | | MDR Subset (n = 21) | | Resistant Subset (n = 31) | |
| | | Combination: | | Combination: | | Combination: |
| | Single agent | Antibiotic + | Single agent | Antibiotic + | Single agent | Antibiotic +., |
| Treatment | treatment | LBP-EC01 v.2 | treatment | LBP-EC01 v.2 | treatment | LBP-EC01 v.2 |
| CK618 | 74.6 | n/a | 74.6 | n/a | 73.1 | n/a |
| Cefdinir | 48.9 | 87.5 | 14.3 | 74.6 | 12.9 | 78.5 |
| Ceftriaxone | 64.4 | 92.4 | 20.6 | 77.8 | 15.1 | 81.7 |
| Cephalexin | 49.2 | 87.9 | 14.3 | 76.2 | 11.8 | 81.7 |
| Ciprofloxacin | 36.4 | 83.0 | 12.7 | 77.8 | 14.0 | 78.5 |
| Cotrimoxazole | 42.8 | 85.6 | 19.0 | 82.5 | 25.8 | 83.9 |
| (TMP/SMX) | | | | | | |
| Fosfomycin 8 | 85.2 | 93.9 | 90.5 | 95.2 | 87.1 | 94.6 |
| Fosfomycin 64 | 93.2 | 98.5 | 96.8 | 100.0 | 95.7 | 100.0 |
| Nitrofurantoin | 83.0 | 93.9 | 85.7 | 98.4 | 90.3 | 97.8 |

TABLE 29
Host Range of LBP-EC01 v.2 and Antibiotics Alone and in Combination on 3-4-Isolate panel

| | Host Range % on all | | Host Range % on | | Host Range % on Beta-lactam- | |
|---|---|---|---|---|---|---|
| | Strains (n = 304) | | MDR Subset (n = 93) | | Resistant Subset (n = 91) | |
| | | Combination: | | Combination | | Combination: |
| | Single agent | Antibiotic + | Single agent | Antibiotic + | Single agent | Antibiotic + |
| Treatment | treatment | LBP-EC01 v.2 | treatment | LBP-EC01 v.2 | treatment | LBP-EC01 v.2 |
| CK618 | 70.7 | n/a | 68.8 | n/a | 68.1 | n/a |
| Ciprofloxacin 0.25 | 34.2 | 80.9 | 11.8 | 77.4 | 14.3 | 75.8 |
| Cotrimoxazole 2 | 46.4 | 81.9 | 17.2 | 75.3 | 29.7 | 78.0 |
| Cephalexin 16 | 57.6 | 85.5 | 24.7 | 79.6 | 8.8 | 79.1 |
| Fosfomycin 8 | 87.5 | 97.0 | 83.9 | 96.8 | 86.8 | 96.7 |

These data demonstrate that LBP-EC01 v.2 outperforms all of the antibiotics tested with the exception of fosfomycin. The current higher level of efficacy may be due to more limited clinical use in recent years thus reducing the overall likelihood of inducing resistance. Additionally, LBP-EC01 v.2 acts in a complementary fashion with antibiotics. Regardless of isolate MDR status or antibiotic identity, in all cases concurrent treatment with LBP-EC01 v.2 plus antibiotic demonstrates a higher host range than treatment with either LBP-EC01 v.2 or antibiotic alone. In the case of Bactrim (TMP/SMX or cotrimoxazole), the percent of sensitive MDR strains from the 304-isolate panel increases from 17.2% when treated with Bactrim alone to 75.3% when treated with Bactrim+LBP-EC01 v.2, providing a strong argument for the benefit of concurrent treatment.

### Example 18: Efficacy of LBP-EC02 with Multiple Twice-Daily Doses and Co-Administration with TMP/SMX (Study 21-LOC-029)

Study 21-LOC-029 was performed to evaluate the clinical relevance of LBP-EC01 as a concomitant therapy with an antibiotic commonly used to treat uUTI. Specifically, the efficacy of CK570 see Table 1B for cocktail composition) was evaluated in a mouse model of UTI compared with TMP/SMX (Bactrim), as well as the co-administration of CK570 plus Bactrim, and a staggered dosing regimen consisting of CK570 followed by Bactrim 1 hour later.

Mice that were treated with CK570 received 1.7×108 PFU/dose via IV administration. Mice that were treated with Bactrim received 0.63 mg/dose by IV administration.

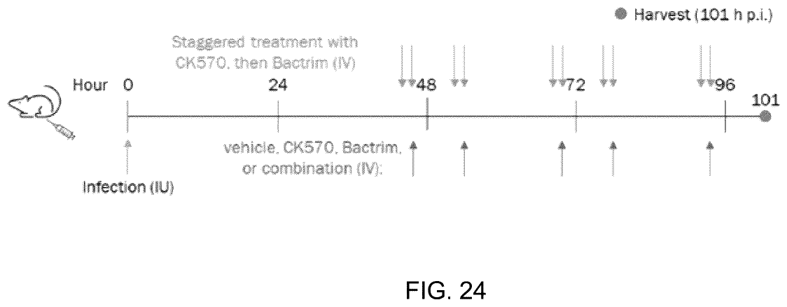

Female C3H/OuJ mice were infected with E. coli (ATCC 700928) at 1×109 CFU by IU administration. Beginning at 46 hours post-infection (p.i.), mice received vehicle (1×TBS), CK570 (Group 2), Bactrim (Group 3), simultaneous co-administration of CK570 and Bactrim (Group 4), or staggered administration of CK570 followed by Bactrim 1 hour later (Group 5) (FIG. 24).

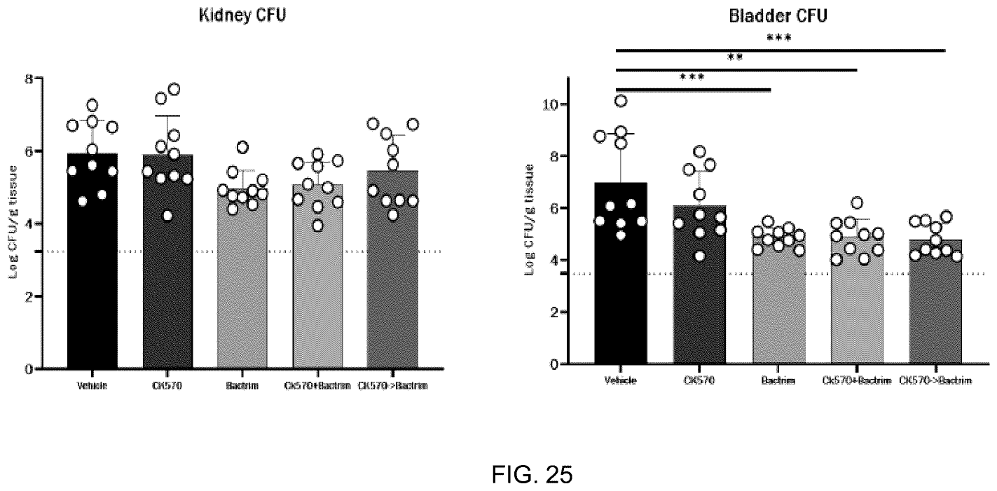

In bladder, either simultaneous co-administration or staggered dosing of CK570 plus Bactrim led to a significant, ˜2 log decrease in bacterial burden relative to the vehicle control (FIG. 25). These results confirm that repeat dosing with a combination of CK570 and Bactrim demonstrated a marked reduction in bacterial load with no observable tolerability or safety signals in a rodent model of E. coli UTI; furthermore, CK570 does not reduce the performance of the antibiotic, Bactrim, in vivo.

Enumeration of the cocktail bacteriophage in bladder, kidneys, and blood showed that the bacteriophage were clearly detectable in the kidneys, but were near the limit of detection in blood (FIG. 26). This observation suggests that PFU levels from blood are not directly predictive of bacteriophage efficacy in the target tissues (kidneys and bladder) in the murine ascending infection model of UTI. Very low PFUs were observed in the bladder in this experiment, with the exception of the CK570 monotherapy group only. Note that slightly lower observed PFUs in the staggered treatment group relative to the CK570 monotherapy and simultaneous co-administration groups are expected due to the fact that animals in the staggered treatment group received bacteriophage one hour earlier than the other groups.

### Example 19: Efficacy of Intravenous Administration of LBP-EC01 v.2 with or without an Intraurethral Loading Dose of LBP-EC01 (Study 21-LOC-032)

Study 21-LOC-032 was performed to inform the route of administration for clinical use of LBP-EC01 by evaluating the impact of a single IU loading dose of bacteriophage cocktail (CK618) on the efficacy of a 5-day BID IV dosing regimen in a murine UTI model.

Mice that were treated with CK618 received 0.5×1010 PFU/dose via IU administration or 1.0×1010 PFU/dose via IV administration.

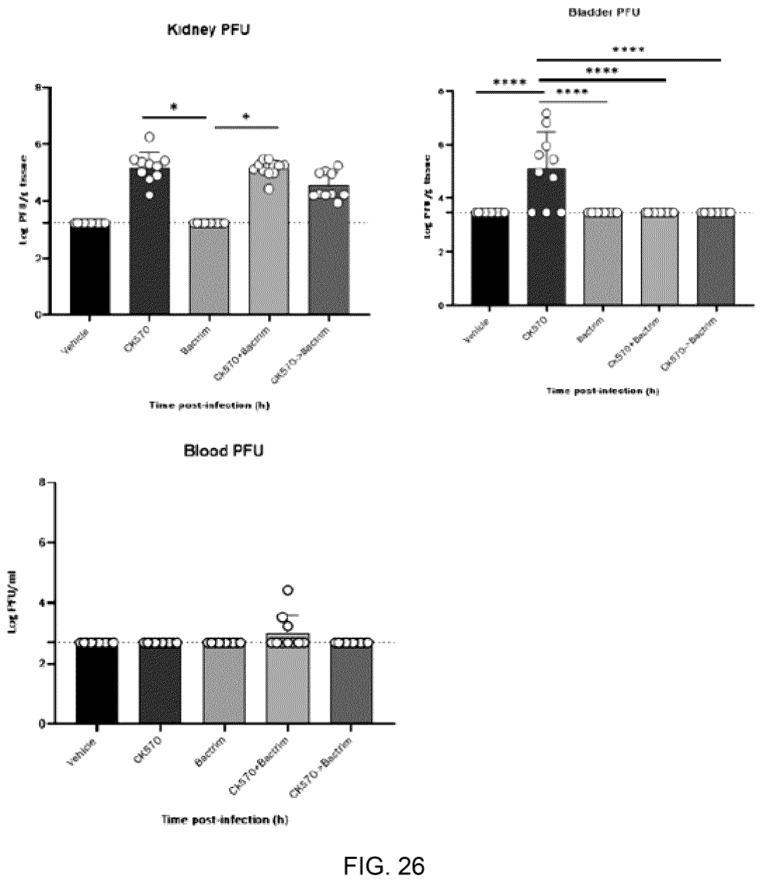

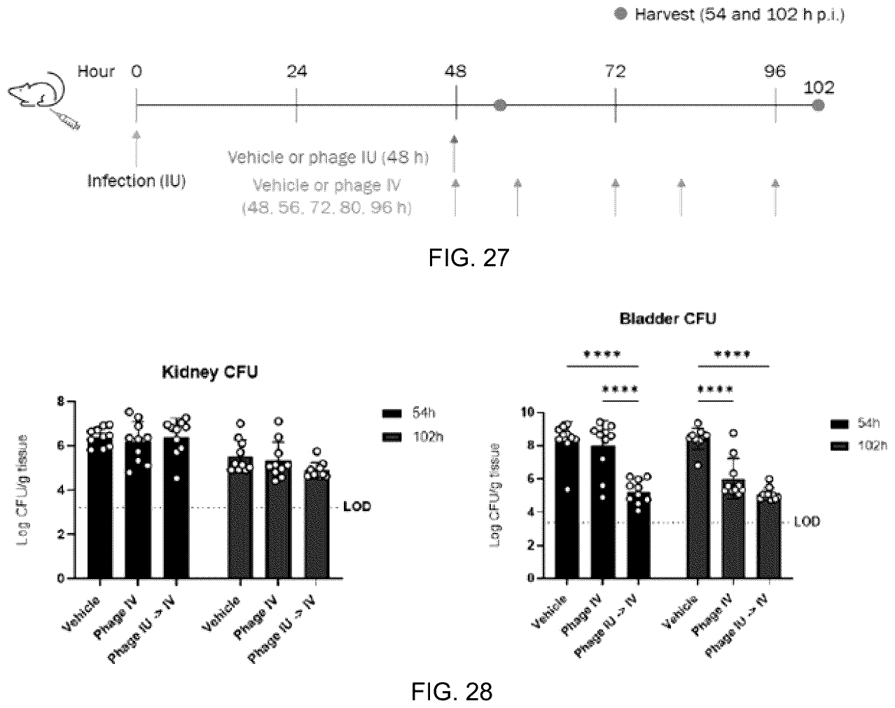

Female C3H/OuJ mice were infected with E. coli (ATCC 700928) at 2×1010 CFU by IU administration. At 48 hours p.i. mice received vehicle (1×TBS) or CK618 by IU administration, followed immediately by IV administration of vehicle or CK618 at 48, 56, 72, 80, and 96 hours p.i (FIG. 27).

In bladder, administration of both an IU dose and an IV dose of CK618 at 48 hours p.i. led to a significant, ˜3 log decrease in CFU at 54 hours relative to either the vehicle treatment or the IV dose alone (FIG. 28). By contrast, at the 102 hour timepoint, both treatment groups that received 5 IV doses of CK618 showed a statistically significant, 2 to 3 log decrease in CFU relative to the vehicle-treated group, with no significant difference between the groups that did versus did not receive an initial IU dose of CK618. Therefore, an initial IU dose improved the efficacy of a single IV dose of CK618 but did not provide significant incremental benefit relative to five IV doses administered BID.

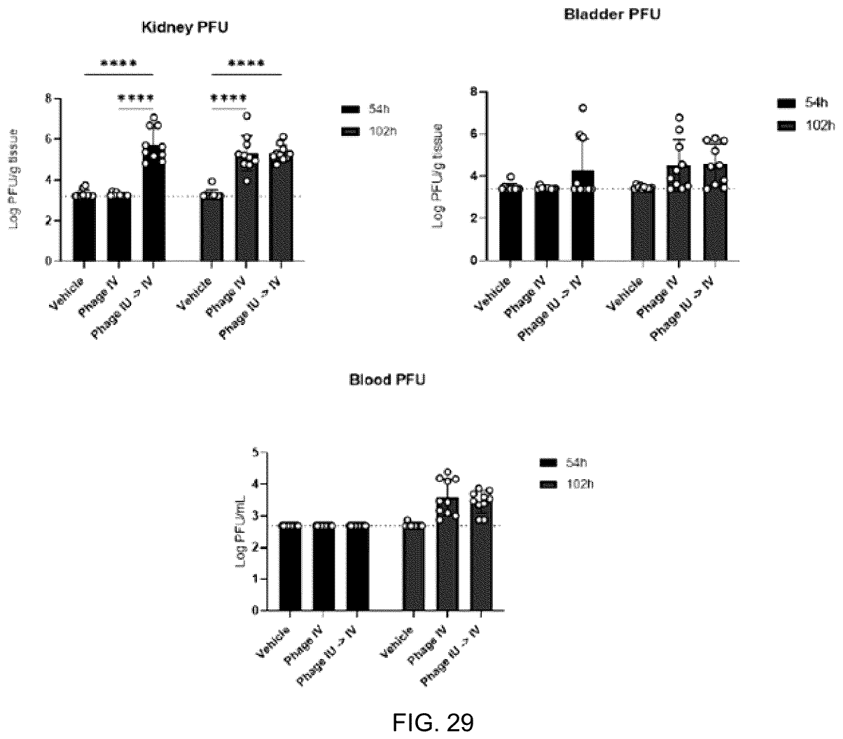

Enumeration of the cocktail bacteriophage showed very few detectable bacteriophage at 54 hours in the kidneys of animals treated only with a single IV dose of CK618 at 48 hours p.i., whereas bacteriophage were clearly detectable in kidneys at the 102 hour timepoint, after 5 IV doses (FIG. 29). By contrast, animals that received an IU dose at 48 hours p.i. prior to IV dosing had clearly detectable bacteriophage in kidneys at both timepoints. In bladder and blood, the IU loading dose did not cause a significant change in biodistribution at either timepoint relative to IV dosing alone. Consistent with the efficacy data, these results suggest that an IU loading dose may improve the biodistribution of bacteriophage to the urinary tract relative to a single IV dose, but not relative to 5 BID IV doses.

Together, these data indicate that an initial IU bacteriophage dose provides more rapid reduction of bacterial burden at early timepoints, relative to IV dosing alone, likely through direct exposure to bacteriophage at the site of infection. However, after 5 BID IV doses, the initial IU loading dose no longer provides any significant additional benefit in terms of either PK or PD.

### Example 20: Non-GLP Comparison of Efficacy of Two Bacteriophage Cocktails Following Five Twice Daily Intravenous Doses in an Acute E. coli Urinary Tract Infection Model in Female, C3H/OuJ Mice (Study 21-LOC-035)

Study 21-LOC-035 was performed to evaluate the efficacy of CK618 in combination with TMP/SMX (Bactrim) in a murine UTI model compared to the individual agents; CK618, Bactrim, and LBP-EC01 from the Phase 1b study.

Mice that were treated with CK618 received 1.0×109 PFU/dose via IV administration. For the LBP-EC01 from the Phase 1b study treatment, animals received the Phase 1 Clinical Trial Material. Bactrim was formulated at a concentration of 6.3 mg/mL.

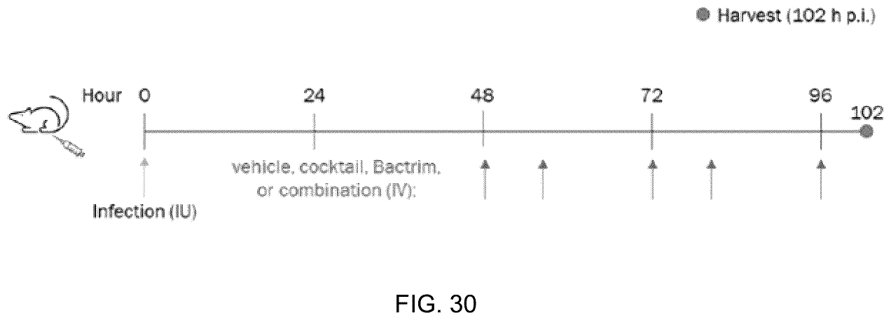

Female naïve C3H/OuJ mice were infected with E. coli (ATCC 700928) at 2×1010 CFU by IU administration. Mice were administered test articles BID by IV at 48, 54, 72, 78, and 96 hours p.i (FIG. 30).

In both kidneys and bladder, administration of the CK618 with Bactrim led to a significant decrease in bacterial burden relative to both vehicle and LBP-EC01 from the Phase 1b study. Furthermore, in bladder, treatment with CK618 or Bactrim as monotherapy also led to a significant decrease in bacterial burden relative to both vehicle and LBP-EC01 from the Phase 1b study.

### Example 21: a Phase 1 Clinical Trial to Test the Safety and Efficacy of a Bacteriophage Cocktail in the Treatment of Lower Urinary Tract Infections

LBP-EC01-01 is a blend of crPhages that each expresses an identical copy of a CRISPR-Cas3 construct targeting E. coli. The CRISPR construct is integrated into the bacteriophage genome without disrupting any genes necessary for the lytic lifestyle of the bacteriophage. This construct was optimized to leverage the Type I-E or Type I-F endogenous E. coli CRISPR-Cas3 system to degrade a highly conserved gene with function related to cell division and maintenance (the ‘CRISPR array’).

Each bacteriophage was engineered with a similar crRNA cassette that contained two elements: (1) a leuO transcription factor gene expressed from a synthetic promoter and (2) a repeat-spacer-repeat encoding a crRNA targeting the gene expressed by a synthetic promoter. Upon DNA transduction during infection, leuO would be expressed from the bacteriophage genome and subsequently upregulate expression of the endogenous Type I-E CRISPR-Cas3 operon in E. coli. Concurrently, the synthetic targeting crRNA would be expressed from the bacteriophage genome that is recognized and processed by the endogenous Type I-E CRISPR-Cas3 protein complex. This crRNA is then loaded onto a CRISPR-Cas3 complex and thereby directs the targeting and degradation of target bacterial DNA. Similarly, the synthetic targeting crRNA would be expressed from the bacteriophage genome that is recognized and processed by the endogenous Type I-F CRISPR-Cas3 protein complex in strains that contain the Type I-F system independently of leuO expression.

TABLE 30

| Element name (5′ to 3′) | Purpose | Source |
|---|---|---|
| Leader sequence | Promoter activity for Type I-E and | Endogenous Type I-F leader |
| | Type I-F CRISPRs | sequence from E. coli strain NC101 |
| Type I-F repeat sequence | Drive maturation of Type I-F | Endogenous Type I-F consensus |
| | spacer sequence to active crRNA | repeat sequence from E. coli strain |
| | | NC101 |
| Type I-F spacer sequence | Guide Cas effector complex to | Designed from E. coli |
| | degrade ftsA gene in E. coli | K12::MG1655 ftsA gene |
| Type I-F repeat sequence | Drive maturation of Type I-F | Endogenous Type I-F consensus |
| | spacer sequence to active crRNA | repeat sequence from E. coli strain |
| | | NC101 |
| Scrambled sequence | Separate Type I-F and Type I-E | Artificially generated |
| | repeats | |
| Type I-E repeat sequence | Drive maturation of Type I-E | Endogenous Type I-E consensus |
| | spacer sequence to active crRNA | repeat sequence from E. coli |
| | | K12::MG1655 |
| Type I-E spacer sequence | Guide Cas effector complex to | Designed from E. coli |
| | degrade ftsA gene in E. coli | K12::MG1655 ftsA gene |
| Type I-E repeat sequence | Drive maturation of Type I-E | Endogenous Type I-E consensus |
| | spacer sequence to active crRNA | repeat sequence from E. coli |
| | | K12::MG1655 |
| BBa_J23102 | Promoter activity for leuO coding | Registry of Standard Biological |
| | sequence | Parts:BioBricks |
| leuO coding sequence | Constitutive expression of leuO | Endogenous leuO sequence from |
| | | E. coli K12::MG1655 |

LBP-EC01-leuO, Type I-E and Type I-F ftsA Targeting CRISPR RNA Sequences

```
(SEQ ID NO 169)
GATAATTAGTGCTGCGGGTAGGTAAGGATAAAAAAGGGTGGCAGCAGGAGATTGAGATGGTTTTGCTTTA
  Leader sequence
TTAACAACGGGCTAAACGTGTAGTATTTGAGCTAGCGAATTCGAGCTCGGTACCACCTCGAGTTCCCCGC
GCCAGCGGGGATAAACCGCTGAAGTAGAAAAACGTGTTACAGCATCAGTCGAGTTCCCCGCGCCAGCGGG
                                                                                                Type I-E repeat-
spacer-repeat
GATAAACCGGGAGAGAGTGAGCGATCCTCCGTTAACATAGTTCACTGCCGTACAGGCAGCTTAGAAAGTA
TTATTCGACGGCGGTGGGATTGCTTCACGTTCACTGCCGTACAGGCAGCTTAGAAAATCAAAATTGCTGT
                                                                      Type I-F repeat-spacer-repeat
CTGCCAGGTGATCGCTTTGACAGCTAGCTCAGTCCTAGGTACTGTGCTAGCATAAAGGAGGTAAATAATG
                                                                                                                        BBa_J23102
CCAGAGGTACAAACAGATCATCCAGAGACAGCGGAGTTAAGCAAACCACAGCTACGCATGGTCGATCTCA
ACTTATTAACCGTTTTCGATGCCGTGATGCAGGAGCAAAACATTACTCGTGCCGCTCATGTTCTGGGAAT
GTCGCAACCTGCGGTCAGTAACGCTGTTGCACGCCTGAAGGTGATGTTTAATGACGAGCTTTTTGTTCGT
TATGGCCGTGGTATTCAACCGACTGCTCGCGCATTTCAACTTTTTGGTTCAGTTCGTCAGGCATTGCAAC
TAGTACAAAATGAATTGCCTGGTTCAGGTTTTGAACCCGCGAGCAGTGAACGTGTATTTCATCTTTGTGT
TTGCAGCCCGTTAGACAGCATTCTGACCTCGCAGATTTATAATCACATTGAGCAGATTGCGCCAAATATA
                                                                                                                                    leuO
coding sequence
CATGTTATGTTCAAGTCGTCATTAAATCAGAACACTGAACATCAGCTGCGTTATCAGGAAACGGAGTTTG
TGATTAGTTATGAGGACTTCCATCGTCCTGAATTTACCAGCGTACCATTATTTAAAGATGAAATGGTGCT
GGTAGCCAGCAAAAATCATCCAACAATTAAGGGCCCGTTACTGAAACATGATGTTTATAACGAACAACAT
GCGGCGGTTTCGCTCGATCGTTTCGCGTCATTTAGTCAACCTTGGTATGACACGGTAGATAAGCAAGCCA
GTATCGCGTATCAGGGCATGGCAATGATGAGCGTACTTAGCGTGGTGTCGCAAACGCATTTGGTCGCTAT
TGCGCCGCGTTGGCTGGCTGAAGAGTTCGCTGAATCCTTAGAATTACAGGTATTACCGCTGCCGTTAAAA
CAAAACAGCAGAACCTGTTATCTCTCCTGGCATGAAGCTGCCGGGCGCGATAAAGGCCATCAGTGGATGG
AAGAGCAATTAGTCTCAATTTGCAAACGCTAA
```

36 patients were randomized 2:1 to receive either LBP-EC01 (1.5×10¹⁰ to 3.0×10¹⁰ PFU/vial dosed BID by intraurethral administration) or inert placebo given twice daily for 7 days by intravesical catheter. A new catheter was installed at the beginning of the study and will be replaced at the discretion of the investigator. Study duration for patients was be up to 56 days, which includes up to 21 days for screening, 7 days of Investigational Medicinal Product (IMP) treatment, a Day 14 assessment (14 days after first dose), a Day 28 assessment (28 days after first dose), and at end of study (35 days after first dose).

Study LBx-1001 was completed on 19 Nov. 2020 where a total of 36 patients were randomized 2:1 including 24 patients to the LBP-EC01 group and 12 patients to the placebo group. The majority of patients were female (31 [86.1%]), White (33 [91.7%]), and not Hispanic or Latino (18 [50.0%]). The mean age of patients was 67.9 years.

Of the 36 patients who were randomized, 34 (94.4%) patients completed the intravesicular study treatment. Two (5.6%) patients in the LBP-EC01 group did not complete the study treatment, with one patient discontinuing treatment due to a non-drug related AE (1 [4.2%] patient) and the other patient withdrawing from the study (1 [4.2%] patient). The majority of patients in both treatment groups were exposed to IMP (1.5 to 3.0×1010 PFU/dose of LBP-EC01 or placebo) BID for 7 days (except for Day 7 where only one morning dose was given) with a mean duration of 6.8 days for the LBP-EC01 group and 7.0 days for the placebo group.

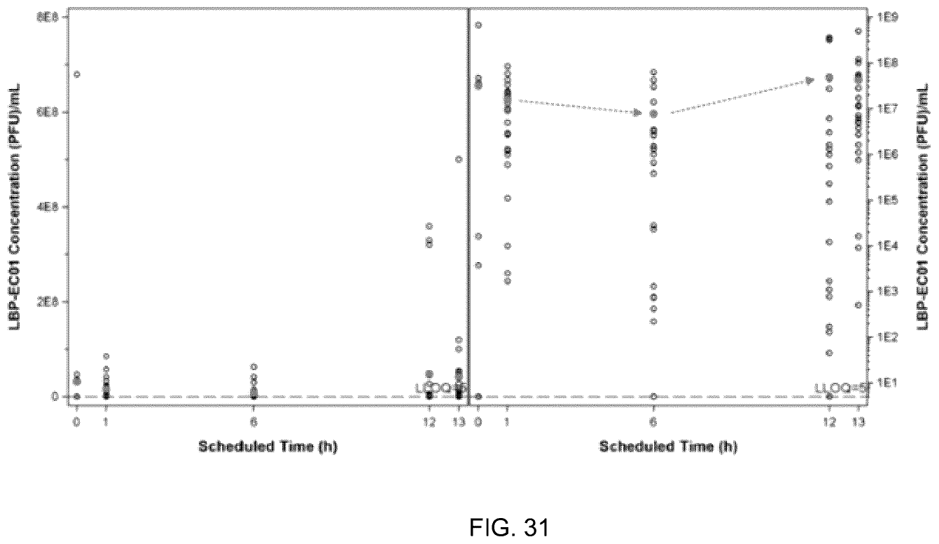

The mean LBP-EC01 urine concentrations were in the range of 7.8·106 to 4.8·107 PFU/mL on Day 1 following Dose 1. Amplification of LBP-EC01 was observed most prominently during Day 1 dosing of LBP-EC01 naïve patients as demonstrated by the increase in PFU/mL concentrations of LBP-EC01 between 6 hour and 12 hour (pre-PM dose) timepoints shown by the red dashed line (see FIG. 31) while patients were allowed to urinate between the 1 hour and 12 hour timepoints. The 0 hour timepoint had low but measurable levels of LBP-EC01 for 5 subjects which is believed to be due to contamination of samples during the initial collection of samples at the clinical site or at the testing laboratory. The mean LBP-EC01 concentrations were in the range of 1.1·106 to 8.9·107 PFU/mL during Days 1 to 28. The mean urine maximum concentration (Cmax) for Dose 1 was 5.7·107 PFU/mL and the mean urine time to maximum concentration (Tmax) for Dose 1 was 3.60 hours. The mean overall exposure, as determined by area under the concentration versus time curve (AUC0-t), was 1.5×108 h*PFU/mL. There was an increase of the mean Cmax in both pre-dose and post-dose values compared with the mean Cmax for Dose 1.

At baseline, the median E. coli burden was 5.3×105 CFU/mL for the LBP-EC01 group compared with 5.6×105 CFU/mL for the placebo group. At Day 7 (End of Treatment), the median E. coli burden was 1.1×106 CFU/mL for the LBP-EC01 group compared with 5×106 CFU/mL for the placebo group. The reduction in urinary E. coli burden from baseline occurred as early as Day 2 in 11 (52.4%) patients in the LBP-EC01 group compared with 2 (28.6%) patients in the placebo group. Subsequently, reductions were observed in:
8 (38.1%) patients in the LBP-EC01 group and 2 (28.6%) patients in the placebo group at Day 3;
10 (47.6%) patients in the LBP-EC01 group and 4 (57.1%) patients in the placebo group at Day 5;
9 (42.9%) patients in the LBP-EC01 group and 3 (42.9%) patients in the placebo group at Day 7/End of Treatment;
8 (38.1%) patients in the LBP-EC01 group and 5 (71.4%) patients in the placebo group at Day 14; and
9 (42.9%) patients in the LBP-EC01 group and 3 (42.9%) patients in the placebo group at Day 28/Early Termination.

The mean (standard deviation [SD]) time to 1-logarithmic reduction in E. coli from baseline was 5.9 (7.33) days for the LBP-EC01 group and 8.4 (5.41) days for the placebo group. The mean (SD) time to recurrence (defined as a past biological elimination followed by an increase above the patient's baseline CFU/mL value), which is not uncommon in this patient population, was 14.0 (0.00) days for the LBP-EC01 group (Table 31)

TABLE 31

| | Parameter | |
|---|---|---|
| | LBP-EC01 | Placebo |
| Statistic/Category | (N = 21) | (N = 7) |

Time to 1 log reduction in
E. coli from baseline (days)

| n | 15 | | 5 | |
|---|---|---|---|---|
| Mean (SD) | 5.9 | (7.33) | 8.4 | (5.41) |
| Median | 3.0 | | 7.0 | |
| Minimum | 2 | | 2 | |
| Maximum | 28 | | 14 | |
| Biological elimination [1] | 7 | (33.3) | 3 | (42.9) |
| at any time point, n (%) | | | | |
| Recurrence [2] of E. coli | 3 | (14.3) | 0 | (0.0) |
| colonization, n (%) | | | | |

Time to recurrence [2] (days)

| n | 3 | | 0 | |
|---|---|---|---|---|
| Mean (SD) | 14.0 | (0.00) | NA | |
| Median | 14.0 | | NA | |
| Minimum | 14 | | NA | |
| Maximum | 14 | | NA | |
| Infection based on clinical signs and | 4 | (19.0) | 0 | (0.0) |
| symptoms [3] at any time point, n (%) | | | | |

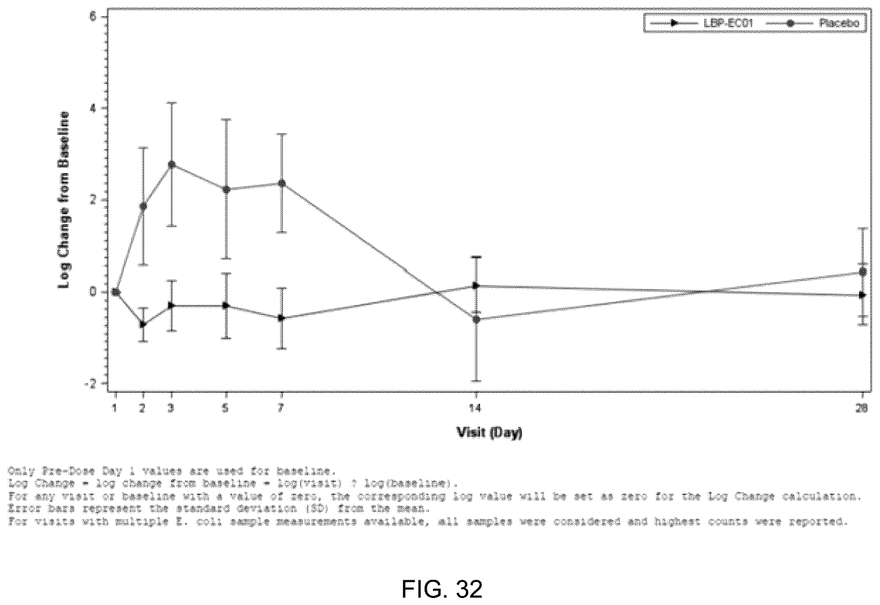

2 to 3-log (100× to 1,000×) separation of mean CFU/mL was observed between the LBP-EC01 and placebo treated groups during the Treatment Period after baseline (Day 2-7) as seen in FIG. 32. This effect was not evident on D14 and D28 and is suggestive that exposure to LBP-EC01 created selective pressure on E. coli during exposure, but patients may have returned to baseline levels of colonization due to the fact that these patients are chronically and asymptomatically colonized and tend to return to that state without the selective pressure.

Across the study results there is no evidence to suggest that the bacteriophage therapy induced either bacteriophage or antibiotic resistance. Of note, 10 subjects in the treatment group were found to have MDR positive isolates at baseline. Most of these isolates (9 of 10) were either strain sequence type ST1193 or ST131. Nine out of 10 patients with MDR isolates demonstrated a 1-log reduction or were lost (potential bacterial eradication) in at least one observed timepoint over the treatment period.

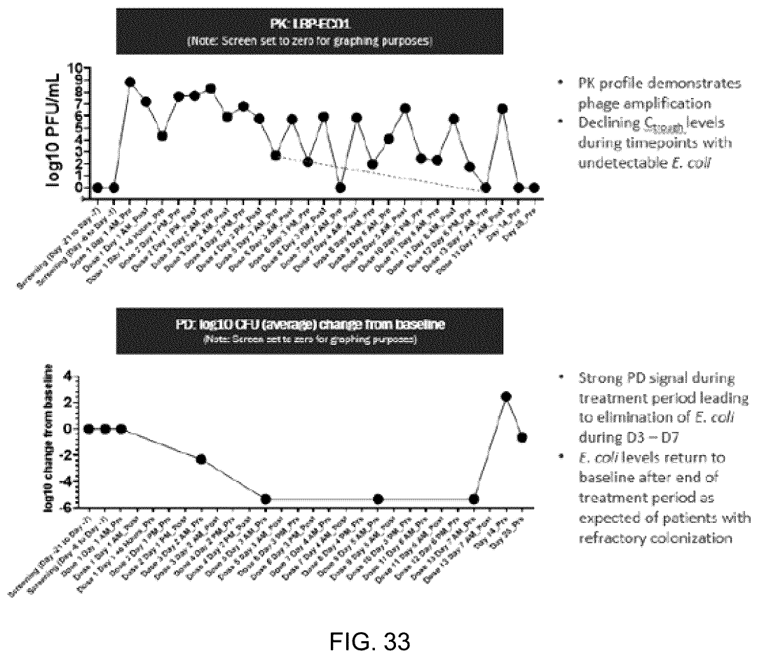

In addition, we observed a positive correlation in patients with sensitive E. coli between their PK Ctrough levels of bacteriophage and the levels of E. coli in their urine over time. For example, Subject 106-012 (FIG. 33) shows elevated levels of both E. coli and bacteriophage prior to Day 3 after which the E. coli levels are reduced significantly out to Day 7 at which time a corresponding decrease in Ctrough levels of bacteriophage is observed. This decreasing level of drug is likely a result of the diminishing ability for the bacteriophage to amplify and persist without a viable E. coli host.

### Example 22: A Two-Part, Phase 2/3 Clinical Trial to Test the Efficacy of Bacteriophage Cocktail in the Treatment of Urinary Tract Infections

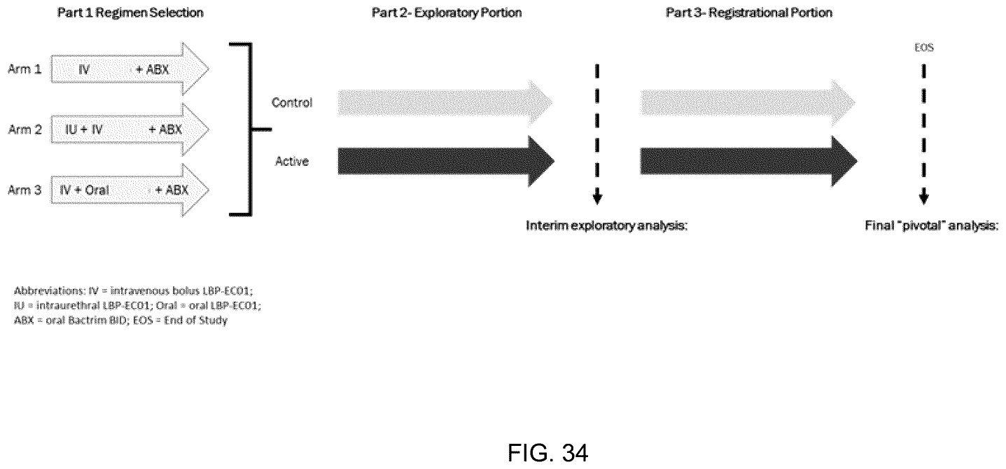

A phase 2/3 clinical trial is performed to test the efficacy of a bacteriophage cocktail on subjects with urinary tract infections. The study design is depicted in FIG. 34

### Inclusion Criteria:

Patients must have a history of recurrent urinary tract infections (UTI) with more than 2 UTIs in the past 6 months or more than 3 UTIs in the past 12 months, with at least one caused by E. coli, as well as an active uncomplicated urinary tract infection (uUTI) infective.

### Protocol:

Subjects will be administered the bacteriophage cocktail along with 800 mg sulfamethoxazole (SMX) with 160 mg trimethoprim (TMP) administered orally.

### Part 1: Part 1 of the Clinical Trial has 3 Arms

Arm 1: the bacteriophage cocktail (1×10¹⁰ to 1×10¹³) plaque forming units [PFU]) IV is given as a 6 milliliter (mL, at a concentration of 1×10⁹ to 1×10¹² PFU/mL) bolus twice daily (4 h apart) from D1 through D5 concomitantly with oral TMP/SMX twice daily (12 h apart) from D1 through D3 (6 doses). Patients should drink 240 mL of water at each LBP-EC01 dosing timepoint.

Arm 2: the bacteriophage cocktail (1×10¹⁰ to 1×10¹³ PFU) IU is given as a single dose of 100 mL (at a concentration of 1×10⁹ to 1×10¹² PFU/mL) via newly placed urinary catheter on D1 and the bacteriophage cocktail (1×10¹⁰ to 1×10¹³ PFU) IV given as a 6 mL (at a concentration of 1×10⁹ to 1×10¹² PFU/mL) bolus twice daily (4 h apart) from D1 through D5 concomitantly with oral TMP/SMX twice daily (12 h apart) from D1 through D3 (6 doses). Patients should drink 240 mL of water at each bacteriophage cocktail dosing timepoint.

Arm 3: the bacteriophage cocktail (1×10¹⁰ to 1×10¹³ PFU) IV is given as a 6 mL (at a concentration of 1×10⁹ to 1×10¹² PFU/mL) bolus twice daily (4 h apart) on D1 and D2 concomitantly with oral TMP/SMX twice daily (12 h apart) from D1 through D3 (6 doses). Patients should drink 240 mL of water at each LBP-EC01 IV dosing timepoint. The bacteriophage cocktail (1×10¹⁰ to 1×10¹³ PFU) oral will be given as a 20 mL liquid (at a concentration of 1×10⁹ to 1×10¹² PFU/mL) with 240 mL water given twice daily (4 h apart) from D3 through D5. Oral calcium carbonate (1000 milligrams [mg]) will be administered approximately 10 to 20 minutes prior to each oral dose of the bacteriophage cocktail.

The regimen, biodistribution, and exposure will be evaluated through serial collection of blood and urine to determine the level of the bacteriophage cocktail measured by a quantitative plaquing assay from pre-dose to over 48 h after the last dose of the bacteriophage cocktail. Patients in Part 1 will be followed until D21

Part 2: Part 2 is designed as a double-blind, randomized, active-controlled, parallel-group, multicenter study in patients with uUTI. Part 2 will test the efficacy, safety, tolerability, and pharmacokinetics of the bacteriophage cocktail when used concomitantly with TMP/SMX, compared with placebo when used concomitantly with TMP/SMX, for the treatment of acute uUTI caused by MDR E. coli in patients with a history of prior E. coli infections of the urinary tract. Part 2 will also assess the recurrence of UTI in patients with confirmed E. coli infection of the urinary tract.

Part 2 will consist of 5 days (D1 through D5 of treatment [dosing regimen to be confirmed in Part 1]) with the bacteriophage cocktail concomitantly with TMP/SMX or placebo concomitantly with TMP/SMX with a follow-up period to D21 (V9). TMP/SMX will be given for the first 3 days of treatment (D1 through D3) while the bacteriophage cocktail 1 will be given for up to 5 days (D1 through D5). Patients positive for E. coli as the predominant organism and who have completed the follow-up period will have a 6-month follow-up period where monthly assessment of recurrence will be performed.

### Outcome Measures

### Part 1

Outcome measures for part 1 include selecting the regimen for the bacteriophage cocktail when used concomitantly with TMP/SMX, which optimizes the pharmacokinetics for the bacteriophage cocktail; assessing the immunogenicity of the bacteriophage cocktail via assessment of NAb levels; and determining the efficacy of the bacteriophage cocktail on resolution of uUTI symptoms and demonstration of microbiologic response of uUTIs in patients with a history of prior E. coli infections of the urinary tract.

### Part 2

Outcome measures for part 2 include determining the efficacy of the bacteriophage cocktail when used concomitantly with TMP/SMX, compared to placebo when used concomitantly with TMP/SMX, on resolution of acute uUTI symptoms and demonstration of microbiologic response of acute uUTI caused by MDR E. coli.; assessing the impact of the bacteriophage cocktail when used concomitantly with TMP/SMX, compared to placebo when used concomitantly with TMP/SMX, on maintenance of clinical (symptom resolution) and microbiologic success as defined in those randomized patients with MDR E. coli uUTI demonstrating an initial response; determining the efficacy of LBP-EC01 when used concomitantly with TMP/SMX, compared to placebo when used concomitantly with TMP/SMX, on resolution of uUTI symptoms and demonstration of microbiologic response of uUTIs caused by E. coli; assessing the impact of LBP-EC01 when used concomitantly with TMP/SMX, compared to placebo when used concomitantly with TMP/SMX, on maintenance of clinical (symptom resolution) and microbiologic success as defined in those randomized patients with E. coli uUTI demonstrating an initial response; monitoring the patients for 6 months for recurrence of uUTI in patients with a documented history of prior E. coli infections of the urinary tract; comparing the bacteriophage cocktail when used concomitantly with TMP/SMX, to placebo when used concomitantly with TMP/SMX, using the following metrics: patient reported outcomes via the Self-Reported Urinary Tract Infection Symptom Assessment (UTISA) questionnaire; number and duration of hospital, emergency department (ED), outpatient visits due to uUTI or uUTI complications; antibiotic medication usage; rescue medication; and rate and time for recurrence; determining the response rate defined as resolution of clinical symptoms of uUTI and demonstration of microbiologic response of the bacteriophage cocktail when used concomitantly with TMP/SMX, against extended spectrum beta-lactamase (ESBL) E. coli strains; and assessing the impact of the bacteriophage cocktail on the microbiome as assessed by microbiological flora present in urine, vaginal, perianal, and stool samples.

TABLE 32
Promoter Sequences

| | SEQ | |
|---|---|---|
| | ID | |
| Promoter | NO | Sequence |
| prom000011 | 70 | ATTTACGATATTAATGTATCACGAGCTCCGTCAATGGTTACTATTCCAGCC |
| | | GAAGAACTAGATCGTCTTCAGAAAATTGAAGAGCTTCTTTGGGAAATTGA |
| | | ATCTGATTTGCCATCAGGATTAGAATCCTGGATTGATT |
| prom000012 | 71 | AAATATAGCATTGATGATGCTTTTAATTATGAAGAAGAATTCGAAACGGA |
| | | AATTCAATTCTTAATGAAAAAGTATAATCTCAAGCGTCAGGATATTCGTAT |
| | | CCTGGCCGACCACCCATGCGGTGAAGATGTTCTTTATATTAAAGGAAAATT |
| | | TGCCGGATATCTTGATGAATATTTTTATTCTAAAGATATGGGCATTGATAT |
| | | GCATATGAGAGTTGTATAAATAGATATATAATTCAGAGGAGACAATC |
| prom000013 | 72 | AATAACATAGAAAAGATTTATCGTCTTTGTGATAAAATTGAAAAAGAAAA |
| | | GAAATATCTATTTTGTCTATGGCCTATTGTTGACGGAAGAGTAGGCCTAGA |
| | | TGTTCTTGATTATGAAACAGAAGACAAAGTAGATGGCGCAACTTTTGATA |
| | | ACGCTTTGGATGTTATTGATTGGCTCGAAGAAAATTATGTGAGGTAAAT |
| prom000014 | 73 | AAAACATTTAAAATTGTCGTAGAATTTTATAACGGCGAAGGTGTAGTCCAT |
| | | CTTAAAGCGGCCAACCAGTTTGATGCGGTAAGAGTATATTGTCAATGCTTT |
| | | GAAAGTTCTAAACAAGCTATAAAAATTAAAAGTGTTGAGGAAACCAA |
| prom000015 | 74 | GATTTATTTGAGATGTTAGAAGATAATCATTCTACGAATAACCAGAATGAT |
| | | TCTAGTGATTATAAGAAAGAGTACCGTATAGTATTACAGAATTATGGAATT |
| | | GAAGCCCCAGATGCTCTTCTAGAAGAACTAGCTTCATACCATCTTGACCCT |
| | | CCGCCCTGGGCTCCCTGGGCAAAATAATTCAAAAAGTTGTTTACTTTCCTT |
| | | TCTAACGATGATATGATAGCTTCTGAAGTATACGGGAGGCTATC |
| prom000016 | 75 | TAATGATTTAGATTCTAGCGGAAATCCTTCACACACTGCCGGTGGAACAGT |
| | | TGGAACAACGTCAGTAACGCTTGAAAATGCAAATCTTCCTGCGACTAAAA |
| | | CTGACGAAAGAGTTTTAATTGAAGATGAAAATGGATCAGTTATTATTGGA |
| | | GGTTGTCAATATGACCCAGATGAAACTGGTCCTATATATACAAAATATCGT |
| | | GAAGACTATGCAACAACAAACTCTTCACATACTCCTCCTACTAATATTAGT |
| | | AATATTCAACCGTCTATTACTGTATACCGTTGGATAAGGATTGCATA |
| prom000017 | 76 | GGAATGCATGTTGGTGGTGTTCAGGCACAACAAATGTCATACCATAAACA |
| | | TGCTGGTGGTTGGGGTGAATATAACAGAAGTGAAGGCCCATTTGGCGCGT |
| | | CAGTTTATCAAGGATATCTTGGAACTAGAAAATATTCCGACTGGGATAAT |
| | | GCTTCATACTTCACTAATGATGGATTTGAATTAGGTGGACCGAGAGATGCT |
| | | CATGGTACACTTAATCGTGAAGGATTAATTGGTTATGAAACTAGACCATG |
| | | GAATATATCATTAAACTATATTATTAAAGTTCATTACTAAGGATTACAA |
| prom000018 | 77 | AACTTACTTCACTTATAAATGGAAATAATCCTGACGGATCAACTGTTGAAG |
| | | AACGAGGATTAACTAATTCTATAAAAACGAATGAAACCAACATTGCGGCA |
| | | GTCACACATGAAGTAAATACAGCTAAAGACAATATATCCTCTTTACAGAG |
| | | CAGCGTTCAAGCTCTACAAGAAGCAGGTTATATTCCTGAAGCTCCAAAAG |
| | | ATGGCCAAGCTTACGTTCGTAAAGACGGCGAATGGGTACTACTTTCTACCT |
| | | TTTTATCACCAGCATAACATGGGGCCGCAAGGCCCCAAAGGATTTTAA |
| prom000019 | 78 | ATAGTTGGTCATTATCTGAACGATACAATAATCCAGACCATAATTTAGTAG |
| | | GTCGTGTTGTCGGTCAAGATCCAAATGTTAAGCAAGGTGCTTATAATAATC |
| | | GTTGGGTGAAAGACTATGCAACAGCTTTAGCTAAAGAATTGAATGGTCAA |
| | | ATTTTAGCGCGTCACCAGGGAATGATGCTTCCAGGCGGTGTTACGATTGAT |
| | | GGACAACGTTTAATAGAAGAAGCTCGATTAGAAAAAGAAGCATTACGCGA |
| | | AGAATTATACTTACTTGACCCTCCATTTGGAATTTTTGTAGGTTAAT |
| prom000020 | 79 | AACAAATGCTAATGGACGCAGCCAAGATTTTTCTTGAGACGGCAAAGAAT |
| | | GCTGATTCTCCTCGTCACATGGAAGTATTTGCAACTCTTATGGGGCAAATG |
| | | ACTACGACGAACAGAGAAATACTGAAGCTTCACAAAGACATGAAAGATAT |
| | | TACATCTGAACAGGTTGGCACCAAAGGCGCTGTTCCTACAGGTCAAATGA |
| | | ATATTCAGAATGCGACAGTATTCATGGGTTCACCAACAGAATTAATGGAC |
| | | GAAATTGGTGATGCTTACGAGGCTCAAGAAGCTCGTGAGAAGGTGATAA |
| prom000021 | 80 | AGATGCAGTTAAAAATAAGACGTTATTTGAGAAAATGACATCGAGTTTAA |
| | | CTAACGTTCTTGTAGTTTCAAACCCGACAATTTGGATGGTGAAAAACTTTG |
| | | GTGCAACATCTAAGTTTGATGGAAAAACGGAAATATTCGGTCCATGTCAA |
| | | ATCCAGAGTATCAGATTTGATAAAACACCTAATGGTAACTTTAACGGATTA |
| | | GCTATTGCTCCAAATCTCCCTAGTACATTTACTCTCGAGATTACTATGAGA |
| | | GAAATTATCACGTTAAACCGTGCTTCTTTATATGCGGGGACTTTTTA |
| prom000022 | 81 | TAAGGTGACTTATACTTGTAATCTATCTAAACGGGGGACCTCTCTAGTAGA |
| | | CAATCCCGTGCTAAATTGTAGGACTACCCTTTAATAATTTCATCAGGATTA |
| | | GTTACTTACCGTGTAAAATCTGATAAATGGAATTGGTTCTACATAAATGCC |
| | | TAACGACTATCCCTTTGGGGAGTAGGGTCAAGTGACTCGAAACGATAGAC |
| | | AACTTGCTTTAACAAGTTGAAGATATAGTCTGATCTGCATGGTGACATGCA |
| | | GCTGGATATAATTCCGGGGTAAGATTAACGACCTTATCTGAACATA |
| prom000023 | 82 | GTTTGGTAACACACTTGATTCGCTTTACCAAGATTGGATTACTTATCCAAC |
| | | GACCCCAGAAGCACGTACCACTCGCTGGACACGTACATGGCAGAAAACCA |
| | | AAAACTCTTGGTCAAGTTTTGTTCAGGTATTTGACGGAGGTAACCCTCCTC |
| | | AACCTTCAGATATAGGAGCGATCCCATCTGATAATGGAATAATAGGTAAT |
| | | CTTACTATTCGTGATTTCTTACGAATTGGTAATGTTCGCATTATTCCTGACC |
| | | CAGTGAATAAAACTGTTAAATTTGAATGGGTTGAATAAGAGGTATT |
| prom000024 | 83 | TTATCCATTTAAAGGAAAAATTGACTATAAAGATTTTACAAATCTATCAGT |
| | | ACGTGTTATAGTAACTGAAGTAGACAAAAATCTAACGAAGTTCGAATCTG |
| | | AACTAGAAAAAGTTGTGCATTCATTACGAGTTGTGTCAAAGATTGATAACT |
| | | CTGTCGAGTCAGATGACAGTGAAGAAGTTGAAGTTCAATCTCTTCAGACG |
| | | TTGATGGAAGAATACATTAATGCAATTCCAGACATCACTGATTCTGACCGT |
| | | GAAGCACTTATTCAATATGCAAATCAGTTATATGTAGAGGCAACACA |
| prom000025 | 84 | AGAATATGCTAATGCAGTTTCTGACTATGAGTATTCTGCTCGGGAAAGAG |
| | | GTACAGCTTTCGCAAAGGAAGAAATGAAAATCATGGTTGATGCTCACACA |
| | | AAGCTTCAGAATTTTATTGAAAACGTCATTTAATGGTTTACAAGTTGGCAA |
| | | GATTATGATATAGTAATCTTGCCAACTGCCAAGGAGAAGAGAATGAAAGT |
| | | TTTGTTTGTTGTGTATGTGATGATTCAATATAATTATCCAATGTTTACTTAT |
| | | AATCTGGTGAACAACATTATTGATATTATTCAAAGGAGTATGTAATT |
| prom000026 | 85 | GCCTTAAAAGCAACGGCACTATTTGCCATGCTAGGATTAGCGTTTGCTTTA |
| | | TCTCCACCAATTGAAGCGAATGTCGATCCTCATTTTGATAAATTTATGGAA |
| | | TCTGGTATTAGACACGTTTATATGCTTTTTGAAAATAAAAGCGTAGAATCA |
| | | TCTGAACAGTTCTATAGTTTTATGCGAACGACTTATAAAAATGACCCGTGC |
| | | TCTTCCGATTTTGAATGTATAGAGCGAGGCGCGGAGATGGCACAATCATA |
| | | CGCTAGAATTATGAACATTAAATTGGAGACTGA |
| prom000027 | 86 | CGGTCTGTTATTTAAACGACGATTATGATTATCTTGGCGTTTATAGTTTAA |
| | | GTGATGCACGGTTTAAACGTAATTTACAAAAGTCAAATTTATTTTATATTG |
| | | ATACTACGGTAAAATTTCAGGGCAAGAAATATTTCTTTACTCTTATAGTTG |
| | | ATTCTGAAACGAAGCATGAGAATAAACGTATTCTTAGTAAAAAGAATATC |
| | | TTAACTATTGTTGATGATCTTTTTGATAAATTCGTAGAAAATCCCAATTTTG |
| | | AAAGCGATTTATTACTAGAAAAATTTGTTAAGGAATGTAGAGAAT |
| prom000028 | 87 | TGAAATCGCCAAGAAAGTAGTTGAGTTAGATGATGCTCGTCAAGAACTTG |
| | | CGGTTAAATTGGAATATATCCGTGAAACTCGTGCAGCAAATGCCCTTGGA |
| | | ATTAGTACTGCCGATGATGTAGTTGAAATTGCAGCACTGACTAAGGTTGAT |
| | | ATTGAAGATACCCTTGCTCGAGTTGAAACCTTTAACGGTAATATTTCTGGG |
| | | GTTGAAACTACCTCTGCCGATGTTCAGGAATACATTAATTCTCTGAAATAA |
| | | TGATAAGGGGCTTCGGCCCCTTATACTTGGAGTAAATAGGAATGAAA |
| prom000029 | 88 | AAAATCGTATTAACACTGCATCTAAGCTAAAAGCTGCTGCAGAAGCTATA |
| | | ATAAGTAAACTAGGTAAATAATTTTAAATCCCTATCTAAATGATAGGGCTT |
| | | TTTGGTATCTAGGCCTTTCTGGACCTCTCTAGGCATCATTTAGTTTATACCC |
| | | TTTATAATATATTATCCTATCCTTTAATTGCCAATCCCTGCCCTAGAATTCC |
| | | CTAAAAATTTTTTCACAAAACTGTTTACATCTCTGTTCTTCCATGGTACTAT |
| | | ACAACTATCAACTACTGATACAGAAAACAACTTGGAGAATGAA |
| prom000030 | 89 | ACTGCTATAACTCCGCAAGAATACATGGCGTCTCTTAAAGAAAAATATAA |
| | | TCTTTCTGCAACAGAAACACTTTTCGATTTACCAGAAAACCTTCAACTAAA |
| | | ATTTCAGGTAGAATTTCAAAAATTAGTTCACCCAGAACAAAAACACTTTAC |
| | | TGCAGTCGTTAAGTCAATTAATGCAGATGGATTGACAATTTTCACCCGACA |
| | | AATAGTACTAATTTAAGCAAGGGGCTTCGGCCCCTTAATTGGAGTATAATA |
| | | TATCAAGAGCCTAATAACTCGGGCTATAAACTAAGGAATATCT |
| prom000031 | 90 | TTGAGCGAAAGTACTAAAGATCTGACTGAGTCTCAAAAAGAAAAAGTCTC |
| | | TGCTCTGGTCGAAGGTATGGATTATTCAGATGCATTCTCAAGTAAATTGAG |
| | | TGCAATCGTAGAAATGGTGAAGAAATCTAATAAAGATGAAAGCACTATTA |
| | | CTGAGAGTATAAATACTCCTGATACTGAAGCAGCCGGACTGAATTTCGTC |
| | | ACTGAAGCTGTAGAAGATAAATCTGCACAAGGTGCAGAAGATATTGTAAG |
| | | TGTATATGCGAAAGTCGCATCTCGTTTCTAATTTTAAAGGTTAACACAA |
| prom000032 | 91 | TTACGTAGATGTTTTAATGAATCATGGATGGAAACTTCGCGGTCATCCAAC |
| | | TAAAAATTGTCATATGTTCACTGATGGAGATATTGAAGAGCTTCATGAAAT |
| | | GGCAGAAGCAATAGGAATGAAACGTTCTTGGTTTCAAGATAAACGCATTA |
| | | AACATTATGACTTACACGCTCGCCGACGCCAAAAAGCTGTAGAACTTGGA |
| | | GCTGTAGAAGTATCTCGCCGTGAAGCAGTAAAAATTTGGCGAACGTTAAA |
| | | ATAAATTGTTTACAGAAGGGTAGTAGTGTGATACTATTACCCTATCAAAAC |
| | | AAATGTGAGATTGGAGAATAAA |
| prom000033 | 92 | TGTATTAGTAGGTCCTGTGACAGCTGTATCATTTATAATCCTAATGATTATT |
| | | GGAATAGTTATAGATGTTACTACTGATATTGAATCAGACGCAGTATTTCTG |
| | | TTAGTATTAATTCTTCCATTAGTAGTTCCATTTTTATTAGTACCTGTAAATT |
| | | GGGTAGGATACTGGTATCAAGGAAGACATTATCGTAAACGCGTATGCGAA |
| | | TGGAAAGCTCAGTGTAAAAAGATTAAAAAGGAACATCAGCTTAAACTTGC |
| | | TGCGTATGAATTTAATGAAATTATGAAATTTGTTAAGGAATCACG |
| prom000034 | 93 | CTATTGTAGTGTCAAGGTCAGGTGCTTATTCTGAAATGACTTATAGGAATG |
| | | GCTATGAAGAAGCTATTCGTCTTCAAACTATGGCGCAATATGATGGCTATG |
| | | CTAAATGTTCTACTGTCGGTAATTTTAACTTGACTCCTGGTGTTAAAATTAT |
| | | TTTTAATGATAGTAAAAACCAATTTAAAACAGAATTTTACGTTGATGAAGT |
| | | TATCCATGAATTATCAAATAATAATTCAGTAACTCATCTATATATGTTCAC |
| | | TAATGCAACGAAACTGGAAACAATAGACCCAGTTAAGGTTAAAA |
| prom000035 | 94 | GCTAAAAATGCTCTGACTGCTAACAAACTGGTTGTAGATGGTATTGAATAT |
| | | GATATCTGTGGAGTTCGTGAAGAAAAACCTGGTGTTCTGACTTTCTTCACA |
| | | ATGATTTTTAAATTTAAAGGTGACACAGAATTCAAACAGTTTGATTTTGCC |
| | | CATGAAGATGAAATCGAAGTTCGTAATCTGAACATTAAGTAAGTACTTTAT |
| | | TAGAGCTCTTGAAAAAGAGTGCAAAAAAGTGTTTACTTCTGCTTTAAACAT |
| | | GATACTATAGACCTATCAAATAAATGAACTGAAACGGAGATTAAA |
| prom000036 | 95 | ATGCTCGTGAATTTCTTGACGAAGAAACCGGCGAGATGATTCGCGAAGAA |
| | | AAATCTTGGCGTGCAAAAGATACTAACTGCACTACATTCTGGGGTCCTTTA |
| | | TTTAAGCATCAACCATTCCGAGATGCTATTAAACGTGCTTATCAGTTAGGT |
| | | GCTATTGATAGTAATGAAATTGTTGAAGCTGAAGTTGATGAATTGATTAAC |
| | | TCAAAGGTTGAAAAATTTAAATCTCCAGAAAGTAAAAGTAAATCAGCTGC |
| | | TGATTTAGAAACTGACCTCGAACAGTTAAGTGATATGGAAGAATTTA |
| prom000037 | 96 | TAAGTGATGAAGCTCATTTTAATTATCTGATGGCTGCTGTTCCTCGGGGTA |
| | | AAAGATATGGTAAATGGGCAAAACTGGTTGAAGATTCCACCGAAGTATTG |
| | | ATTATTAAGTTACTTGCTAAGCGGTATCAAGTTAATACAAATGATGCAATT |
| | | AACTATAAATCAATTCTTACTAAAAATGGAAAACTATCTTTAGTATTAAAA |
| | | GAACTAAAAGGTTTAGTCACGTATGATTTTTTGAAAGAAGTGACTAAGAA |
| | | CGTAAAAGAACAGAAACAACTCAAAAAACTAGCATTGGAATGGTAAA |
| prom000038 | 97 | ATCGTAATTAACGGTTTTAATAAAGTAGAAGATTCTGCTCTGACCCGTGTT |
| | | AAATATTCTTTGACTCTTGGTGATTATGATGGTGAAAATACATTTAATTTC |
| | | ATTATCAATATGGCAAATATGAAAATGCAACCAGGAAATTATAAACTTCT |
| | | GCTCTGGGCAAAAGGTAAACAAGGTGCTGCTAAATTTGAAGGTGAACACG |
| | | CGAATTATGTGGTAGCTCTTGAAGCTGATTCTACCCACGATTTTTAATAAA |
| | | GGGCTTCGGCCCTTTATAATTTACACTAAAACTTGAATGAGGAAATT |
| prom000039 | 98 | TTTCAGTTAAAAATCTGCATCACGTTGTTTTAGCACACGGTGTTAAATCTA |
| | | AAATTATTGTATTGCAAACAATAGGTCGCGTATTACGTAAACATGGTTCTA |
| | | AAACAATCGCAACAGTCTGGGACCTCATAGATGACTGTGGTGTCAAGCCA |
| | | AAATCTGCTAATACTAAAAAGAAATATGTTCACTTGAACTATCTTTTAAAA |
| | | CACGGCATTGATCGTATTCAGCGCTACGCAGATGAAAAATTTAATTACGTA |
| | | ATGAAAACAGTCAATTTATAAGGGCTTCGGCCCTTTGGAGAAAAAG |
| prom000040 | 99 | GCCGCTAAGAAAGCTGATAAAGTTGCTGATGATTTGGATGCATTCAATGTT |
| | | GATGACTTCAATACAAAAACTGAAGATGATTTTATGAGCTCAAGCTCTGG |
| | | CAGTTCATCTAGTGCTGATGACACAGACCTGGATGACCTTTTGAATGACCT |
| | | TTAATAGATTATATTACTAATTAATTGGGGACCCTAGAGGTCCCCTTTTTT |
| | | ATTTCAAAAATTTTTCATAAAACGGTTTACATCCTTGTCCTTCCATGGTACT |
| | | ATACAACTATCGGCAATACTGCTGATAATTAAAGAGGAAAATAAT |
| prom000041 | 100 | TATAATAAATTTTGGTCAGAAGACGAAGAAAAAGACCTTTTAAATCTTTTA |
| | | GACTCTTTAAATGACAGAGGAATAAAATTTGGACTGTCGAATGTTTTAGA |
| | | GCACCACGGAAAGGAAAATACTCTTCTTAAAGAATGGTCTAAAAAATACA |
| | | CTGTTAAGCATCTTAATAAAAAATACGTCTTTAACATATATCATTCCAAAG |
| | | AAAAGAATGGAACTGATGAAGTATATATTTTTAATTAATTGCTTATATATT |
| | | CAAATGGTATAATTATTTAACTTATTAATGAATTGAAAGGAAAAATA |
| | | CAAAGCATCACGCTTATGGAATGTTCCAAAATTATTTGCCTACTATGCGAG |
| | | CGAGAGTCAAGGAACTTGGTTATAATATGACCGATGCTGAAATAAAAAGA |
| | | ATGTTGAATAAACGGTCCAATTCAGCTTCCTGGGCGTACATTGAACTTTCT |
| | | TATTGGTTAAATATACATAAGGGCGATATAAGAAAAGCAATATCCTCTTAT |
| | | AATTCGGGATGGAATGTTAAAGCTGGTTCTAAATATGCTTCTGAAGTCCTA |
| prom000042 | 101 | GAAAAGGCTAATTACCTTAAAAATAATAAACTTTTGGAAATAGTAA |
| prom000043 | 102 | AATCAAAGAAACTGAATCAGCATTTCGTTTAGCTTTTGCCAAGGCACATTT |
| | | CATTAAGAAAGTAATTTCAGGTGAAATTGTTGTACAAGGTAAAACTCGCA |
| | | AAGAACTGACCGAAGAACTTTCTAAAATTGATATGTATTCTTCTTATGTTG |
| | | ATAAACTAGTTGGAATGAACATTTTTCATATGACTTCCGACGAAGCAAAG |
| | | AAACTTGCTGAAGAAGCTAAAGCCAAAAAAGAAGAAAACGAATATTGGA |
| | | AAACTACTGATGTAGTTACAGAATACACCAAAGATTTAGAGGAAATCAA |
| prom000044 | 103 | ATTGAATTGAGTTGGTACCAGTTTAAATCTCTTATGACAAATGTTAAAACT |
| | | GTCATTGAAGAAAATCAGGGTCCTGAAAATATTACTATTCGCGAAAAAGC |
| | | TTTAAAGATAGTATACAGTCTTGAAGAAATACAAAAAGATATTGAATCTA |
| | | TGGCAAAATTTATTGATGAACCTATTAATAAAGTTTATATTCAAGATTATA |
| | | CTGTAGGTCAAATTCGCGATTTAGCAAGGAAAATTTA |
| prom000045 | 104 | GAAATAACTAAAGATCAGTTTTATCTTCTTCAAGATAAAGTAAGCGAAATT |
| | | TATGAAATTGCTTATAGCAAAAATCGTGAAACTGTAAAAATAGAATCTAG |
| | | TAAGTTGATGCTTCAATTAGAAGAAATTGAACGAGATTTAATTGCGTTAGA |
| | | ATTCTTTTGTGGTGAAGTGAAAACTGTTACAATCAATGATTATATTTTAGG |
| | | CGAAATTAGCTATCTTTATAAGGCGATTATTA |
| prom000046 | 105 | TCGGAGGGGGAAGAAGAAAGAAAAGAACGTCTTTTTAATGAATCTCTTAA |
| | | GATAATTAAATCTGCCATGGAAAATGTTATCCAGGAGATTGTCATTAAACT |
| | | AGAAGATGGTTCTACACACATTGTGTATGTGACAAAATTAGATTGGGTTG |
| | | ATGGAAAAGTCGTAATGGACTTTGCTGTTCTTGACCAAGAAAGAAAAGCT |
| | | GAGTTAGCTCCTCATGTAGAAAAATGTATTACAATGCAACTACAAGATGC |
| | | ATTTAATAAAAGGTCAAAGAAAAAGTTTAAATTCTTTTAAGGAGTAAGT |
| prom000047 | 106 | GAATATTCAACTGGACAGCATTTATTAACTTTTCCTGAAATAAAACGATAT |
| | | ATTCTGAGAAATAATTTTTCTAATGAAGAGCATATAGTTACTGAATCTATG |
| | | CTTAGGAATGCATTTAAAGCAGAATATACAAAAATAATGTCCAATAGAAA |
| | | TGAAGCTTGGACTGTTACTGATTATTATGACTAAAGGTGTATT |
| prom000048 | 107 | GCAACAACGATGCGATGGACAAATGACTGGGAGTATTCAAAAAATCATAA |
| | | GAAGCCCATGGTGACAAGAAAGGCTCATATGTTAGTGTTAATAGACCGTG |
| | | AGCAGATTAAAGCCCGAGAAGCCCTCCAGAATCATAAAAAGGCTGCCTTT |
| | | GAATGGTTTATGGATAACACTGCTCCTGAGACTAAGAAAGCAGTGAGCGC |
| | | ATGGTTCAGTGGAAAAAATTGTGAAAGAAGTTTCTTTTAGTGGTTTACAAG |
| | | ACTGTTCCTCTGTGGTACTATACAACTATCAACTACGGAGGAACACAAA |
| prom000049 | 108 | GATGTTATTCTTTCAACTGACATGATGCAAAAGCGCGGTCTTTCTGATGAA |
| | | GAAACTTATAAAGCATTATCATGGATGAAAGTCAATGTTAACCGTTTACGC |
| | | AATGTTTCACTGCGTACTGCACTTTATCTTGCTGACTTTATTATGACCGACA |
| | | AAAACGGTTGGCAAGAAATCGCTGAGGTTACTCTTCTGAAATAAATTCAT |
| | | AAGAGGACTTCTATGACAAAAAGGCAGTTCAGAAATAGATTATATGGACT |
| | | GCCATCAAAAAGATGACTAGAATTAAACTGGTAAATGGAGGTAATG |
| prom000050 | 109 | ATTTACGATAAAGTTAAGTCTATTAAAGACCGTAGTGTTATTAAACGTGAA |
| | | GTTGGTATTATTGCTCAGGACCTTGAAAAGGAATTACCGGAAGCTGTATCT |
| | | AAAGTTGAAGTTGATGGATCTGATGTTCTGACAATTTCTAACTCTGCTGTG |
| | | AATGCTCTTTTAATTAAGGCTATTCAGGAAATGAGTGAAGAAATTAAAGA |
| | | ATTGAAAACTCCTTTCTTTACTAAAATTGCTCGCAAAATTAGTAAATATTT |
| | | TAAATTCTAACAACAAGGGGGAAATGCCCCCTTTGGAGTATAAATT |
| prom000051 | 110 | GAACTGTATAAGTTTAACCTATTTTTAGGTAAAACGGCAGAAACTTATAAA |
| | | AATTGGAACAAAGGCGGAAAAGCTCCATGGAGTGATTTTTGGGATGCTAA |
| | | ATCACAGTTTAGTAAAGTGAAAGCACTTCCTGCATCAACATTCCATAAAGC |
| | | ACAGGGCATGTCTGTAGACCGTGCTTTCATTTATACGCCTTGTATTCATTAT |
| | | GCAGATGCTGAATTGGCTCAACAACTTCTTTATGTTGGTGTTACCCGTGGT |
| | | CGTTATGATGTGTTTTATGTATGATTAAATTTGAGGAAGCTATTC |
| prom000052 | 111 | AAAATTGTTATGGGGTGCTTAGCAGTTTGTTTAGTTGCATTAGCAGCAGTT |
| | | CCATTTGTTAGTGTTGAAAATGATACTCAACCTGTGATTGAATCTAGCACC |
| | | GTTATTCACACTAATGGTAAAATATCAGTTAAAATTGATGATAATCTTCAT |
| | | GTGAATACTAATGGAACGCTGGGTGTTCAATTAGGTAATATCTGTGTAAGC |
| | | ACTACTGGAGTAATTACTACTTGTATTTGAGGAAATTATT |
| prom000053 | 112 | ATGCTGGTATTTTATCACTGGTTACTAACGATCGTGGTGCTATTGATGATG |
| | | TTCTTGAGTCTCTCAAAAATAAAGATGTTAAACAACTCAGAGCTTTAGCAC |
| | | CAAAATATGCAGCTGATTATTCGTGGTTCGTAGGTAAACTTGCCGAAGAA |
| | | ATCTATTCACGTGTAACTCCGCAGAGTATTATTCGTATGTACGAAATTGTC |
| | | GGCGAAAATAATCAGTATCATGGTATTGCAGCTAATACTGAATTGCATTTA |
| | | GCTTATCTTTTCATTCAGTTAGCATGTGAAATGCAGTGGAAGTGAT |
| prom000054 | 113 | TAAAGAAATAGATGGTTATACTTATGATATTAATGACGTTTATGTATGTCA |
| | | AAGATTGGAATTTCAATACCAAGGAAATACATTTTATTTTAGACCTCCTGG |
| | | AAAATTTGAACAATTTTTAACTGTAAGCGATATGTTATCCAAATGCTTGCT |
| | | TAAGGTCAATGATGAAGTTAAAGAAATTAATTTTCTTGAGATGCCAGCATT |
| | | TGTTTTAAAATGGGCAAATGATATTTCTACAACTTTAGCAATTCCTGGCCC |
| | | TAATGGTCCAATAACCGGAATTGGCAATATTATTGGATTATTTGA |
| | | ACCAGCAGTTAAAAAAGAACTTGCTTCTAGATTTGCTAAAATTGATGCCAC |
| | | TTATCAAGAGCTTAAGAAAAATCAACCTGAGGCCAAACCTGAAACTTCTG |
| | | CTAAATCACCAGAAGCAAAACAGGTCCAGGTGATTGAAAAGAACAAAGC |
| | | ACAACAAGCTCCTGTTCAACAAGCATCTCCTTCAATCAATAATACTAATAA |
| | | TGTTATTAAGAAAAATACTGTCGTTCATAATATGACACCTGTCACAAGCAC |
| prom000055 | 114 | AACTGCTCCTGGTGTATTTGGTGCGACGGGAGTTAATTAAGGAATAAT |
| prom000056 | 115 | AGTGAAGAAGAGTTTTATTGTTTCCGCGAATATAAAGAACCGACTTCTGA |
| | | AGAGGATGAAGTCAAAGACAAGGTTTCTGGCGTAACAAAAATTCACTGCA |
| | | TTGTTGACGAAAACAATGCAGATGAAATCATTGAACTTTTGCGAAAAACT |
| | | TTCAAAAAGTAGTTTACAGCAGGGTAGTAGTGTGATACTATTACCCTATCA |
| | | AAACTAATGGAGAAAAGAAAATGTTAGCACCTTATATTATGGCAGCAGTT |
| | | ATGTTGGTTTGTTTATATCTTTTGATTAAAGCTTGCTAAGGAGAATAAA |
| prom000057 | 116 | TCATATTGTAGCTAAGATGTGTAATCTTATTCCGGGAGATTTGATATTTTCC |
| | | GGTGGTAATACTCATATCTATATGAATCACGTAGAACAATGTAAAGAAAT |
| | | TTTGCGTCGTGAACCTAAAGAGCTTTGTGAACTGGTAATAGGCGGATTGCC |
| | | TTATAAATTCCGCTATCTTTCTACTAAAGAACAATTGGAATACATTCTTAA |
| | | ACTCAGGCCTAAAGATTTCGTTCTCAAAGATTATCAGTCCCACGGCGTCTT |
| | | GAAAGGAAAAATGGCGGTGTAATTTTAATTTAATTGTGAGGATTT |
| prom000058 | 117 | GCTTCCTGATGCGGTTGAGGAGATGAAAGTCTTTTTAGAAAATCAGCTTGC |
| | | GAAATATGATTGCGATGTGTTCATTAATCAGACTCAACCTAATGTTCATAT |
| | | TAACAACTGTAAATGCTATATCATCGTTAATCCTTTAACGGGAAAACATCG |
| | | TCTTGGAATTAGTAATCCAAATCGTAGCGCATCGGATATGGCAGAAGATG |
| | | TTGAGGCATGCTTTAAAATTTCTAAATCTCCGGCTGAACATCATATTTTAA |
| | | TTAACGGTCTTTCTCAAGACGATATTATAGAGGTTATTAAAACTTT |
| prom000059 | 118 | AATGACTTCAATTGAATTTACTAGTGCCGTTAGAATAGCTCTACAGGAAAT |
| | | GGTTGTAAAATTTATTGCAATTGATTCATTTGAAGACCATCCTACCATAGG |
| | | AAATAAAATACAAGTTAAGTATTTAGATAACCAAGAACATATCTTAGAAC |
| | | AATACTCTGATAAAGGAATTACTTTCAAACAGGAAATAATTTCTCCTTCTA |
| | | AACCTGGGTATGGAACTTGGCAATTATTAGGTGCGCAAACTGTTACGCTA |
| | | GATAGTCACACACAACCTACAGTATTTTATTATTTTGAGAGAGTAGC |
| prom000060 | 119 | TTGCTAATAATAAAGATAAAGTTCTGTATCAGTCCTGTCATATTCTTCAGA |
| | | AAAAAGGACTATACTATATCGTTCATTTTAAAGAAATGCTTCGTATGGATG |
| | | GTCGCCAAGTTGAAATGACAGAAGAAGATGAAGTTCGTCGTGATTCGATT |
| | | GCATGGCTGTTAGAAGATTGGGGACTGATTGAAATCGTTCCTGGTCAAAG |
| | | AACTTTTATGAAAGATTTAACTAATAATTTCCGAGTTATTTCTTTTAAACA |
| | | AAAACATGAATGGAAACTCGTTCCTAAATATACGATTGGTAATTAAT |
| prom000061 | 120 | CTCGGCTTTCTAAATCAAAACAAACCTCTGACGTTAAGCTAACGATTGTAG |
| | | CACTCAAAGCTCGTATTGATGGTTCTCGTATAGCAGAAGGCGCTGAAGTTG |
| | | TTAAATTGAACGTTCTTCTTAAAGGCTCTGATTGGAAAACTGTGAAAAAGT |
| | | TGTCAGAAGCAGAAATGCAATATGATATGTGTGATAAAATTATTCAAGGT |
| | | GTAGAGCGGTATCAAAACTTGTCTTTTATTGATAAACTTAAACTGAAAAGA |
| | | GGATACCCGTTAAATTGTTCAATTTTTAAACTTATCCGAGGATAAT |
| prom000062 | 121 | CTATTAAGCATATTCAAGACATGCGAGCATTTGAGGCTGGAAAATAATGA |
| | | GATATAGCATTGATGATGCTTTTAATTATGAAGAAGAATTTGAAACGGAA |
| | | ATTCAATTCTTAATGAAAAAGCATAATCTTAAGCGTCAGGATATTCGTATC |
| | | CTGGCCGACCACCCGTGCGGTGAAGATGTTCTTTATATTAAAGGAAAATTT |
| | | GCCGGATATCTTGATGAATATTTTTTATTCTAAAGATATGGGCATTGATAT |
| | | GCATATGAGAGTTGTATAAATAGATATATAATTCAGAGGAGACAATC |
| prom000063 | 122 | TTGAGCGAAAGTACTAAAGATCTGACTGAGTCTCAAAAAGAAAAAGTCTC |
| | | TGCTCTGGTCGAAGGTATGGATTATTCAGATGCATTCTCAAGTAAATTGAG |
| | | TGCAATCGTAGAAATGGTGAAGAAATCTAATAAAGATGAAAGCACTATTA |
| | | CTGAGAGTATAAATACTCCTGATACTGAAGCAGCCGGACTGAATTTCGTC |
| | | ACTGAAGCTGTAGAAGATAAATCTGCACAAGGTGCAGAAGATATTGTAAG |
| | | TGTATATGCGAAAGTCGCATCTCGTTTCTAATTTTAAAGGTTAACACAA |
| prom000064 | 123 | TGAAATCCAAAAAATGACCGAAGAAGGTAAAGATTGGAAAACTGAAGAT |
| | | CCAGACAGTAAATACTACCTGCATCGTTACACTCTTCAGAAGATGATGAA |
| | | AGACTATCCAGAAGTTGATGTTCAAGAATCACGCAATGGATACATCATTC |
| | | ATAAAACTGCTTTAGAAACTGGTATCATCTATACCTATCCATAATCATAAT |
| | | CATAAGGGGCTTCGGCCCCTTTCTTCATTTTGAAAGCACACAAAACATACT |
| | | CAGAAAATGATGTATATAATGGCATCAACTCGATAACATGAGATTGATT |
| prom000065 | 124 | GATTGTTAATAACTTCGACCGCATGTACTGGGTAAAATTATTTGCTTTAGA |
| | | TTATCTTGATAAATTTAACTCAGGTTATGATTATTATATCGTTCCTGATACC |
| | | CGTCAAGATCATGAAATGGATGCGGCTAGGGCGATGGGTGCTACAGTAAT |
| | | TCATGTAGTTCGTCCTGGTCAAAAATCCAATGATACACATATTACAGAAGC |
| | | TGGATTGCCAATTCGTGATGGCGATTTAGTAATTACAAACGATGGTTCTCT |
| | | TGAAGAACTTTTTTCTAAAATTAAAAATACACTAAAGGTACTATA |
| prom000066 | 125 | AATTAAAGAAACTGAATCAGCATTTCGTTTAGCCTTTGCCAAGGCGCATTT |
| | | CATTAAGAAAGTAATTTCAGGTGAAATTGTTGTGCAGGGTAAAACTCGCA |
| | | AAGAACTGACCGAAGAACTTTCTAAAATTGATATGTATTCTTCTTATGTTG |
| | | ATAAACTAGTCGGAATGAATATTTTTCATATGACTTCCGACGAAGCAAAG |
| | | AAACTTGCTGAAGAAGCTAAAGCTAAGAAAGAAGAAAACGAATATTGGA |
| | | AAACTACTGATGTAGTTACCGAATACACTAAAGATTTAGAGGAAATCAA |
| prom000067 | 126 | CGGTTTGTTATTTAAACGACGATTTTGATTATCTCGGTGTTTATAGTTTAAG |
| | | TGACGCATGGTTTAAACGTAATTTACAAAAGTCAAATTTATTTTATATTGA |
| | | TACTACTGTAAAATTTCAGGGCAAGAAATATTTCTTTACTCTTATAGTTGA |
| | | TTCTGAAACGAAGCATGAAAATAAACGTATTCTTAGTAAAAAGAATATCT |
| | | TAACTATTGTTGATGATCTTTTTGATAAATTTGTAGAAAATCCCAATTTTGA |
| | | AAGTGATTTATTACTAGAAAAATTTGTTAAGGAATGTAGAGAAT |
| prom000068 | 127 | ATGACTGCTATAACTCCGCAAGAATACATGGCGTCTCTTAAAGAAAAATA |
| | | TAATCTTTCTGCAACAGAAACACTTTTCGATTTACCAGAAAACCTTCAACT |
| | | AAAATTTCAGGTAGAATTTCAAAAATTAATTCACCCAGAACAAAAACACT |
| | | TTACTGCAGTCGTTAAATCAATTAATGCAGATGGAATGACAATTTTTCACC |
| | | GACAAATAGTACTAATTTAAGCAAGGGGCTTCGGCCCCTTATTTGGAGTAT |
| | | AATATATCAAGAGCCTAATAACTCGGGCTATAAACTAAGGAATATCT |
| prom000069 | 128 | AACTTACTTCACTTATAAATGGAAATAATCCTGACGGATCAACTGTTGAAG |
| | | AACGAGGATTAACTAATTCTATAAAAACGAATGAAACTAACATTGCGGCA |
| | | GTCACACATGAAGTAAATACAGCTAAAGACAATATATCCTCTTTACAGAG |
| | | CAGCGTTCAAGCTCTACAAGAAGCAGGTTATATTCCTGAAGCTCCAAAAG |
| | | ATGGCCAAGCTTACGTTCGTAAAGACGGCGAATGGGTACTACTTTCTACCT |
| | | TTTTATCACCAGCATAACATGGGGCCGCAAGGCCCCAAAGGATTTTAA |
| prom000070 | 129 | GTTTGGTAACACACTTGATTCACTTTACCAAGATTGGATTACTTATCCAAC |
| | | GACCCCAGAAGCACGTACCACTCGCTGGACACGTACATGGCAGAAAACTA |
| | | AAAATTCTTGGTCAAGTTTTGTTCAGGTATTTGACGGAGGTAACCCTCCTC |
| | | AACCTTCAGATATAGGAGCGATCCCATCTGATAATGGAATAATAGGTAAT |
| | | CTTACTATTCGCGATTTCTTGCGAATTGGTAATGTTCGCATTATTCCTGACC |
| | | CAGTGAATAAAACTGTTAAATTTGAGTGGATTGAATAAGAGGTATT |
| prom000071 | 130 | GTTAAATCTCTTAAGGACCGTGAAATTATCGGTCATGAGATTGGCATTATC |
| | | GCACAGGATTTACAAGAAATATTACCGGAAGCTGTGAAATCTTCAAAAGT |
| | | TGGCAATCTTGATAATCCAGACGATGTTCTGACAATTTCTAACTCGGCAGT |
| | | GAATGCTCTTTTAATTAAGGCTATTCAGGAAATGAGTGAAGAAATTAAAG |
| | | AATTGAAAACGCCTCTCTTTACTAAAATTGCTCGCAAAATTAGTAAATATT |
| | | TTAAATTCTAACAACAAGGGGGAAATGCCCCCTTTGGAGTATAAATT |
| prom000072 | 131 | TAATGATTTAGATTCTAGCGGAAATCCTTCACATACTGCCGGTGGAACAGT |
| | | TGGAACAACATCGGTGACACTTGAGAACGCAAATCTTCCTGCGACTAAAA |
| | | CTGATGAAAAAGTTTTAATTGAAGATGAAAACGGATCAGTTATTATTGGA |
| | | GGTTGTCAATATGACCCAGACGAAACTGGTCCTATATATACAAAATATCGT |
| | | GAAGACTACGCAACAACAAACTCTTCACATACTCCTCCTACTAATATTAGT |
| | | AATATTCAACCGTCTATTACTGTATACCGTTGGATAAGGATTGCATA |
| prom000073 | 132 | TTAAACATATTCAAGATATGCGAGAATTTGAAGCGGGGCAATAATGCGTT |
| | | ATTCAATTGAAGATGCCTTTAATAATGATGAAGAATTTGAAACAGAAATT |
| | | AAATTCTTAATGGAAAAGTATCGTCTACGACGTCAGGATATTCGTATCCTG |
| | | GCAGACCACCCTTGTGGTGAAGACGTCCTTTATATTAAAGGGAAGTTTGCT |
| | | GGGTACATCGACGAGTATTTTTATACAAAAGATATGGGTATCGATATGTGC |
| | | ATGCGAGTGGTATAAATAGATATGTAATTTCAACTTAGGAGGCAATC |
| prom000074 | 133 | TTATTAATGAAAGCGTAAAAGACCTGACTGAATCTCAGAAAGAAAAAGTT |
| | | ATTGGCCTGGTTGAAGGTATGGATTATTCTGACGCATTTGGTACTAAGTTA |
| | | ACTGCTATTGTAGAAATGGTTAAAGGTTCTACTAAAGAAGAAGCCGCTAT |
| | | TACCGAAAGTATAAATACAGTTGACAATGACGCTGCCGGTCTTAATTTTGT |
| | | CGCCGAAGCAGTTGACACTACCACAACTCAGGTAGAACAGAATTCTAATG |
| | | TAAGTTTATATGCGAAAGTCGCATCTCGTTTCTAATTTAAAGGTTAACACA |
| | | A |
| prom000075 | 134 | CACTGTTCCAGGACTTAATGGAATGGCCTCAATGATGTCTGCCTGGAGTGC |
| | | CGTAGCATCCCGCTATAACATTACTGAAGAACAGCAGAAAGAAATATTTA |
| | | AAGAACTCCCTTTATTTGAGGATATGGTTGCTGGTGATTCAGGTGGCAGCC |
| | | CTCAAGCTATTGCATCAGGAACAACTACAGGTGCAGTCGTAAATAAAGGT |
| | | CCAGAACAAATTCCTTCCAAGAAAAGAAAGCGAATTAAAGTAAATATTAA |
| | | TAAGTTGTGATATGATGGCCTTATTAATTTAAGGCCTCTGGAGAATACT |
| prom000076 | 135 | CTTCGGAAGGGGAAGACATTCGCAAAGAACGAGTGTTTAATGAATCTCTT |
| | | AAAATTATCCGTTCAGCAATGGAAAATGTCATTCAAGAGATTCTGATTACA |
| | | CTTGAAGACGGTTCCCATCATATAGTCTATATTACAAAACTAGACTGGGTT |
| | | GATGGTAAAATCGTAATGGACTTTGCAGTTCTTGACCAGGACAGAAAAGC |
| | | AGAACTTGCACCGCATGTTGAAAAATGTGTTACAATGCAAGTACAACAAG |
| | | CTTTTAATGAACGAACCAAGAAAAAATTTAAATTCTTTTAAGGAGTTATC |
| prom000077 | 136 | GGTATGACCGCCGACGAATTTGTTGTACAATTACGTACTACTCTCCGGAAT |
| | | GCGATTTCCGACCAATTGGCTATTGCAGAGTATAAAGATGACCCCACTGTT |
| | | GGGACTAAATTACAAATAACATATTTAGATAACCAAAGACACGTACTCCC |
| | | AACGTATTCCTCTTATGGTATCACGGTTTCTCAAGAAATAGTGTCTCAGGC |
| | | TAAGTCCGGGTACGGCACTTGGAATTTACTAGGTGCGCAAACCATTACTCT |
| | | TGACAACCATATTTCACCTACAACCTTTTATTATTTTGTGAGAACCGC |
| prom000078 | 137 | ACCGGAAGAAAAAGCGTTCAAATGGGGACTATGCAGATCAGTTTCTGTTG |
| | | AAAAAGACCATGAATTCACTATTGCTGCGGCAACTGATGGAACGCCATGG |
| | | AAAACTAAGCAAATCGCCGGATTTATTAGCGACCGTTGGGTAGAAGATGG |
| | | TGTTAAGCTTTATAACATCGTTTTCGCTGGTCAGTTCATGGTAGTTCCTGAA |
| | | TTCCAAATTAAAAAATATAGCCCAGCTGCTTTCGCATAAAGTTGTTTACTT |
| | | CTCCTCTGGTTTTGATATTATAACCATATCAACCAGAGGAGAATAAT |
| prom000079 | 138 | GTCAATGCTCAAGTTTATCTGGAATTCCCTATGGAAGGCTGGTAATGATTA |
| | | ACAAAACATATTGGTCTAATATAGACGACGGTAATGAAGGTATTTCGTAC |
| | | TATTCAGTAGATTGGACAGGATGTCCATCCTACTTAGTGAAACGTATTTGT |
| | | CAAGAATTTAAAATCGACCAGCCAGCAGGTATTATCGTGAACAGATACAT |
| | | TAATGGATATCAACCTATCGATGAAGTAATAAGAACAACTCAAGCTGAGC |
| | | TTGAAAAGTTTTTCAATAAGTGCCAATGGATAACAGGATGGATTATAT |
| prom000080 | 139 | AAAGAAATGCTGCGTATGGACGGACGTCAGGTTGATATTGATGGTGAAGA |
| | | CTATCAGCGTCGTGATTCGATTGCTCAGTTGCTTGAAGACTGGGGATTGAT |
| | | TGTTATTGAAGACTCTGCTCGTGAAGACCTGTTTGGCCTGACTAATAACTT |
| | | CCGTGTTATCTCTTTTAAACAGAAAGATGACTGGACTTTGAAAGCAAAATA |
| | | CACAATTGGTAATTAAAGCAATGGGACTTCGGTCCCATTTGGAGTATAATA |
| | | AGTTCATCAACAAACAAAAGACAATAACTCGTCTAAAGGAAATTAA |
| prom000081 | 140 | GGTATGCATGTTGGTGGTGTCCAAGCACAGCAAATGTCATATCATAAACA |
| | | CGCTGGTGGATGGGGCGAATATCAACGTCATGAGGCACCATTCGGAGCAT |
| | | CCGTATATCAAGGTTATTTGGGTACTAGAAAATACGCTGATTGGGATAAC |
| | | GCATCATATTTCACAAATGACGGCTTCGAATTAGGTGGTCACCGCGACGC |
| | | AACAGGCACTCTTAACCGTGAAGGACTTATCGGATATGAGACTCGTCCAT |
| | | GGAATATATCTTTAAATTATATTATTAAAGTCCATTACTAAGGGTAAAAA |
| prom000082 | 141 | AGAACGCTCAAGAGAAATCTAAACTATTGGGCGGTTATACTTATCTGCTTA |
| | | AAAACTCTGTTACAGACGAAGTTAAACCGTCCGCAGGTTTAATTGCTCAG |
| | | GAAGTTCAAGAAGTATTACCTGAACTTGTTTCTGAAGATAAAGAGACCGG |
| | | ACTGCTTCGTTTGAACTATAACGGTATTATTGGTTTAAATACAGCTGCAAT |
| | | AAACGAGCATACAGATGAAATCAAGGAATTGAAATCTGAAATTACCGAAT |
| | | TGAAAGCATTAATTAAATCATTGATAAAATAATAAAAGGGCCTTCGGGCC |
| | | CTGGAGGTTTAT |
| prom000083 | 142 | TCCATGCAGAATTGAATGCAATTTTGTTTGCCGCTCGTAAAGGTAATTCTA |
| | | TTGAAGGTGCTACGTTATATACGACGCTTTCTCCATGTCCTGATTGTACTA |
| | | AAGCTATTACGCAGTCAGGTATTAAAAAGGTCGTATACGCAGAATTATAC |
| | | GACCGATCTCCTGAAAATTGGGCAGATATTCTAAAACAAGCTGGTATTGA |
| | | AGTAATTCAGTACTCTCGTAATAATCTTCGTTCTCTGAATTGGGAACAAAT |
| | | CCGAAACTTTTGTGGTGAATAATATGATTTTAACTGAACAGGAAACT |
| prom000084 | 143 | AGTGGTATTAAAATTAAGTTCCGTCAACCTAAATTATTTGATGATAAAAAT |
| | | ATCGCGTTAATGATTGCTTCATGTATTGAGGCTGTTTTTGTCAATGGAGAG |
| | | TCCATCCCTGTTGAAGATTTAACTGAAGCAGAAATAAACGACCTTTATGGT |
| | | CTTATAACAGAAGATGACATGATCAATATTAAAAATCTTTTGGTGTCGCCA |
| | | TCAATTTATCTGGCAACCCCTATTAAATGTCCCAAATGCGGGGCGTCACAC |
| | | GTTCACACAATACGAGGCCTCAAAGAATTCTTTGAGTTACTATA |
| prom000085 | 144 | TGAAATCCAAAAAATGACCGAAGAAGGTAAAGATTGGAAAACTGACGAT |
| | | CCAGATAGTAAGTACTACTTGCATCGTTACACTCTCCAGAAGATGATGAA |
| | | AGACTATCCAGAAGTTGATGTTCAAGAATCGCGTAATGGATACATCATTC |
| | | ATAAAACTGCTTTAGAAACTGGTATCATCTATACCTATCCATAATCATAAT |
| | | CATAAGGGGCTTCGGCCCCTTTCTTCATTTTGAAAGCACACAAAACATACT |
| | | CAGAAAATGATGTATATAATGGCATCAACTCGATAACATGAGATTGATT |
| prom000086 | 145 | GGAATGCATGTTGGCGGTGTTCAAGCACAACAGATGTCATACCATAAACA |
| | | TGCTGGTGGTTGGGGAGAATACCAAAGACATGAAGCTCCATTTGGCGCGT |
| | | CCGTTTATCAAGGATATCTTGGAACTAGAAAATATGCCGACTGGGATAAT |
| | | GCTTCATACTTCACTAATGATGGATTTGAATTAGGTGGACCGAGAGATGCC |
| | | CTTGGTACACTTAATCGTGAAGGATTAATTGGTTATGAAACTAGACCATGG |
| | | AATATATCATTAAACTATATTATTAAAATTCATTACTAAGGATTACAA |
| prom000087 | 146 | GTTTGGTAACACACTTGATTCGCTTTACCAAGATTGGATTACTTATCCAAC |
| | | GACCCCAGAAGCACGTACCACTCGCTGGACACGTACATGGCAGAAAACCA |
| | | AAAACTCTTGGTCAAGTTTTGTTCAGGTATTTGACGGAGGTAACCCTCCTC |
| | | AACCTTCAGATATAGGAGCGATCCCATCTGATAATGGAATAATAGGTAAT |
| | | CTTACTATTCGCGATTTCTTGCGAATTGGTAATGTTCGCATTATTCCTGACC |
| | | CAGTGAATAAAACTGTTAAATTTGAATGGGTTGAATAAGAGGTATT |
| prom000088 | 147 | CGGTTTGTTATTTAAACGACGATTTTGATTATCTCGGTGTTTATAGTTTAAG |
| | | TGACGCATGGTTTAAACGTAATTTACAAAAGTCAAATTTATTTTATATTGA |
| | | TACTACTGTAAAATTTCAGGGCAAGAAATATTTCTTTACTCTTATAGTTGA |
| | | TTCTGAAACGAAGCATGAAAATAAACGTATTCTTAGTAAAAAGAATATCT |
| | | TAACTATTGTTGATGATCTTTTTGATAAATTTGTAGAAAATCCCAATTTTGA |
| | | AAGTGATTTATTACTAGAAAAATTTGTTAAGGAATGTAGAGAAT |
| prom000089 | 148 | CGGTTTGTTATTTAAACGACGATTTTGATTATCTCGGTGTTTATAGTTTAAG |
| | | TGACGCATGGTTTAAACGTAATTTACAAAAGTCAAATTTATTTTATATTGA |
| | | TACTACTGTAAAATTTCAGGGCAAGAAATATTTCTTTACTCTTATAGTTGA |
| | | TTCTGAAACGAAGCATGAAAATAAACGTATTCTTAGTAAAAAGAATATCT |
| | | TAACTATTGTTGATGATCTTTTTGATAAATTTGTAGAAAATCCCAATTTTGA |
| | | AAGTGATTTATTACTAGAAAAATTTGTTAAGGAATGTAGAGAAT |
| prom000090 | 149 | TTGAGCGAAAGTACTAAAGATCTGACTGAGTCTCAAAAAGAAAAAGTCTC |
| | | TGCTCTGGTCGAAGGTATGGATTATTCAGATGCATTCTCAAGTAAATTGAG |
| | | TGCAATCGTAGAAATGGTGAAGAAATCTAATAAAGATGAAAGCACTATTA |
| | | CTGAGAGTATAAATACTCCTGATACTGAAGCAGCCGGACTGAATTTCGTC |
| | | ACTGAAGCTGTAGAAGATAAAGCTGCACAAGGTGCAGAAGATATTGTAAG |
| | | TGTATATGCGAAAGTCGCATCTCGTTTCTAATTTTAAAGGTTAACACAA |
| prom000091 | 150 | AAATTCTGGAATCAGCGTGATATTCGTACCAGAATTGAAGCACTTCTGCTT |
| | | GTTCGTGATATGACCACGTGTCCTCTTCCAAAAGGAACTTTAGATGGATTC |
| | | GTTGCGCATGATTCTATTCATGACTGTGCGAAAGACATCCTGATGATGAAG |
| | | TACGCTTTGCGATATGCTATGGGTCTTGAAGATGCTCCATCAGAGGAAGAT |
| | | TGCGATCCTCTATCTCTTCCAACAAAACGATAAAAAGTTGTTTACTTCCTC |
| | | GGTTAGTTGTGGTATTATAACACCATAGCTACTGAGGATAATAAA |
| prom000092 | 151 | GAAGAGGGGGAAGAAGAAAGAAAAGAACGTCTTTTTAATGAATCTCTTAA |
| | | GATAATTAAATCTGCCATGGAAAATGTTATCCAGGAGATTGTCATTAAACT |
| | | AGAAGATGGTTCTACACACATTGTGTATGTGACAAAATTAGATTGGGTTG |
| | | ATGGAAAAGTCGTAATGGACTTTGCTGTTCTTGACCAAGAAAGAAAAGCT |
| | | GAGTTAGCTCCTCATGTAGAAAAATGTATTACAATGCAACTACAAGATGC |
| | | ATTTAATAAAAGGTCAAAGAAAAAGTTTAAATTCTTTTAAGGAGTAAGT |
| prom000093 | 152 | GATTGTTAATAACTTCGACCGCATGTACTGGGTAAAATTATTTGCTTTAGA |
| | | TTATCTTGATAAATTTAACTCAGGTTATGATTATTATATCGTTCCTGATACC |
| | | CGTCAAGATCATGAAATGGATGCGGCTAGGGCGATGGGTGCTACAGTAAT |
| | | TCATGTAGTTCGTCCTGGTCAAAAATCCAATGATACACATATTACAGAAGC |
| | | TGGATTGCCAATTCGTGATGGCGATTTAGTAATTACAAATGATGGTTCTCT |
| | | TGAAGAACTTTTTTCTAAAATTAAAAATACACTAAAGGTACTATA |
| prom000094 | 153 | AATGACTTCAATTGAATTTACTAGTGCCGTTAGAATAGCTCTACAGGAAAT |
| | | GGTTGTAAAATTTATTGCAATTGATTCATTTGAAGACCATCCTACCATAGG |
| | | AAATAAAATACAAGTTAAGTATTTAGATAACCAAGAACATATCTTAGAAC |
| | | AATACTCTGATAAAGGAATTACTTTCAAACAGGAAATAATTTCTCCTTCTA |
| | | AACCTGGGTATGGAACTTGGCAATTATTAGGTGCGCAAACTGTTACGCTA |
| | | GATAGTCACACACAACCTACAGTATTTTATTATTTTGAGAGAGTAGC |
| prom000095 | 154 | TAATGATTTAGATTCTAGCGGAAATCCTTCACACACTGCCGGTGGAACAGT |
| | | TGGAACAACATCGGTGACACTTGAGAACGCAAATCTTCCTGCGACTAAAA |
| | | CTGATGAAAAAGTTTTAATTGAAGATGAAAACGGATCAGTTATTATTGGA |
| | | GGTTGTCAATATGACCCAGACGAAACTGGTCCTATATATACAAAATATCGT |
| | | GAAGACTACGCAACAACAAACTCTTCACATACTCCTCCTACTAATATTAGT |
| | | AATATTCAACCGTCTATTACTGTATACCGTTGGATAAGGATTGCATA |
| prom000096 | 155 | aaagcgaggtgctggtgttcgccgcggccaattctgactgggcgccacccgaggaactcaagcagttcgccca |
| | | ggccgcccgcgaagtgatgcgcctgcacccggatgattgcctgtccatggcggccatcgagcggtattttcgc |
| | | atactgtactggcagaagggcgcggaggagttggatgcgggtaacctgctcggcctgattgagagaggccggc |
| | | tcgatggcctgccctacgagactttggccaccaagttccgcatgatcgacagccttcaactgccggtgatcat |
| | | cccatttg |
| prom000097 | 156 | cagggctggcgccgacgatacggtgggcaagcatctggatatcttcaaccgtcgcgcacggaaggggcagtgc |
| | | ttccatacaccctgcctaggcgtgcgcgagtttccggccagttttcggttgctggaagagggcagtgccgagc |
| | | ctgaagtcgatgcctttctgcgcggcgagcgtgatctgggctggatgctgcatgacattgacttcgccgatgg |
| | | catgaccccgcacttcttccgtgccctgatgcgcgatgggctgatcgaggtgccggccttcagggcggcagag |
| | | gacaaggc |
| prom000098 | 157 | tcaagacgctgcaccggcagatcttggttggggaaggcgaccccggtctgcaggccttgctccagttcctcga |
| | | ctgttggcagccggagcagttcaagcccccgctgttcagcgaagcaatgctcgacagcaacttagtgttccgc |
| | | ctagacggccaacaacgctatctgcacgagactccggcggccctggcgttgcgtacccggctgttggccgacg |
| | | gcgacagccgcgaggggctgtgcctagtctgcggccaacgtcagccgttggcgcgcctgcatccagcggtcaa |
| | | gggcgtca |
| prom000099 | 158 | cgacagggtcaatgccactatccgtgaccgctactacggtgccgcgtccagcacgccagccacggttttcccg |
| | | atactgctgcgcaacacacaaaaccacttggccaagctgcgcaaggagaagcccggactagcagtgaacctag |
| | | agcgcgatataggcgaaatcattgacggtatgcagagccaattcccgcgttgcctgcgcctggaggaccaggg |
| | | acgctttgctattggttactaccaacaggcccaggcccgtttcaaccgtggccccgattccgtcgagtaagga |
| | | gcagaaga |
| prom000100 | 159 | GGCATTGATATGCATATGAGAGTTGTATAAATAGATATATAATTCAGAGG |
| | | AGACAATC |
| prom000101 | 160 | TGCATATGAGAGTTGTATAAATAGATATATAATTCAGAGGAGACAATC |
| prom000106 | 170 | ACTGCAACCATGAAAATTGGTTCTGTACTGAAAGCTGACAACACAGAAGC |
| | | TACTGCTGCTGGTGAAGCTGTTAAAGTTCTGCTGTGGACTGACGCACTGTT |
| | | CGGGATCGATGATGTTGCAGTAGGTGACACTTTCACCGCAGTTGTTGGTGT |
| | | ACGTGACCTGACTCTCAACCGCTATGCAGTTTACTACAAGAGTGGTGATCT |
| | | GATTGATGACGCTGGCGTTGCAGCACTGGAAACTCACGATCTGAAACTGA |
| | | CTGAAAAAGTTGTTTTCACTTCTTAATTTTTAATAGGAGATTTATAC |

### Example 23: Colicin Characterization and Selection on an E. coli Clinical Validation Panel

Colicins can act through many different mechanisms including pore formation in the inner membrane, nuclease activity, blocking translation, and inhibition of peptidoglycan synthesis (Tables 34-35).

Inducible colicin-encoding plasmids were transformed into bacteria cells. Bacteria cells containing the colicin-encoding plasmid were cultured. Twenty-seven colicin proteins, including Colicin Ia, Colicin Tb, Colicin E1, Colicin M, Colicin 10, Colicin D, Colicin E2, Colicin E3, Colicin A, Colicin B, Colicin K, and Colicin U, were generated from plasmid-based expression and were tested for ability to suppress growth on a panel of E. coli clinical isolates.

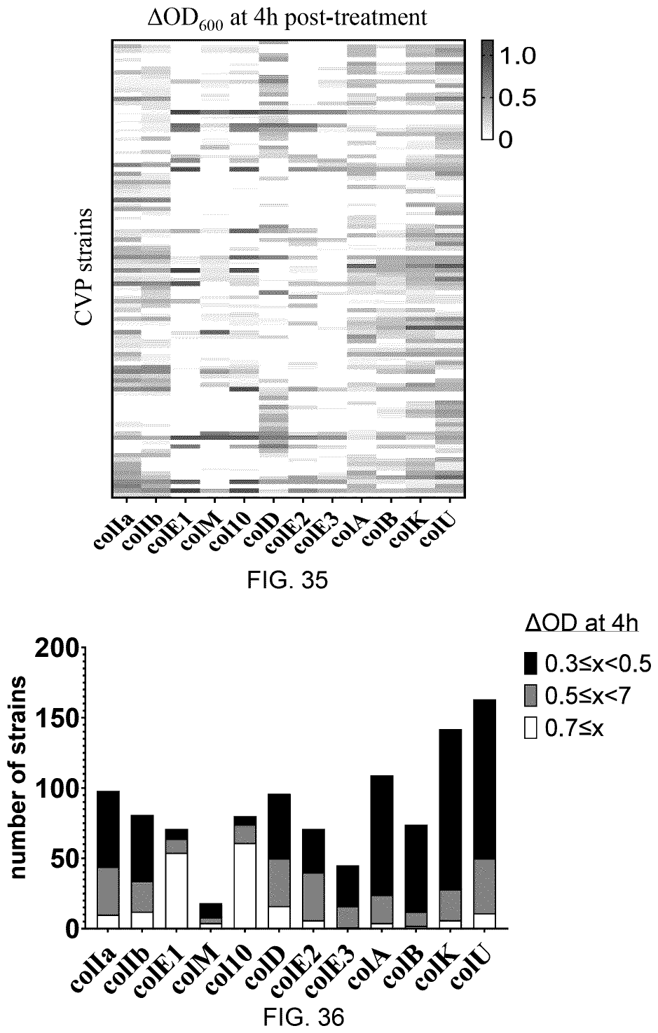

Concentration of E. coli cell cultures on a clinical validation panel were calculated based on spectrophotometer readings of Optical Density at 600 nm (OD₆₀₀). Four hours after treatment, effective killing by the colicin was determined by measuring the ΔOD₆₀₀ with and without colicin treatment (FIG. 35). Greater differences in ΔOD₆₀₀ indicated more effective killing by the colicin (FIG. 36). Multiple colicins were identified that exhibit broad activity against the E. coli clinical validation panel.

TABLE 34
Example Colicin Gene Sequences

| | SEQ | |
|---|---|---|
| Gene | ID NO | Sequence (5′-3′) |
| Colicin Ib | 161 | ATGTCTGACCCTGTACGTATTACAAATCCCGGTGCAGAATCGCTGGGATATG |
| | | ATTCAGATGGCCATGAAATTATGGCCGTTGATATTTATGTAAACCCTCCACG |
| | | TGTCGATGTCTTTCATGGTACCCCGCCTGCATGGAGTTCCTTCGGGAACAAA |
| | | ACCATCTGGGGTGGAAACGAGTGGGTCGATGATTCCCCAACCCGAAGTGAT |
| | | ATCGAAAAAAGGGACAAGGAAATCACAGCGTACAAAAACACGCTCAGCGC |
| | | GCAGCAGAAAGAGAATGAGAATAAGCGTACTGAAGCTGGAAAACGCCTTT |
| | | CTGCGGCAATTGCTGCAAGGGAAAAAGATGAAAACACACTGAAAACACTC |
| | | CGTGCCGGAAACGCAGATGCCGCTGATATTACACGACAGGAGTTCAGACTC |
| | | CTGCAGGCAGAGCTGAGAGAATACGGATTCCGTACTGAAATCGCCGGATAT |
| | | GATGCCCTCCGGCTGCATACAGAGAGCCGGATGCTGTTTGCTGATGCTGATT |
| | | CTCTTCGTATATCTCCCCGCGAGGCCAGGTCGTTAATCGAACAGGCTGAAA |
| | | AACGGCAGAAGGATGCGCAGAACGCAGACAAGAAGGCCGCTGATATGCTT |
| | | GCTGAATACGAGCGCAGAAAAGGTATTCTGGACACGCGGTTGTCAGAGCTG |
| | | GAAAAAAATGGCGGGGCAGCCCTTGCCGTTCTTGATGCACAACAGGCCCGT |
| | | CTGCTCGGGCAGCAGACACGGAATGACAGGGCCATTTCAGAGGCCCGGAAT |
| | | AAACTCAGTTCGGTGACGGAATCGCTTAAGACGGCCCGTAATGCATTAACC |
| | | AGAGCTGAACAACAGCTGACGCAACAGAAAAACACGCCTGACGGCAAAAC |
| | | GATAGTTTCCCCTGAAAAATTCCCGGGGCGTTCATCAACAAATCATTCTATT |
| | | GTTGTGAGTGGTGATCCGAGGTTTGCCGGTACGATAAAAATCACAACCAGC |
| | | GCGGTCATCGATAACCGTGCAAACCTGAATTATCTTCTGACCCATTCCGGTC |
| | | TGGACTATAAACGCAATATTCTGAATGACCGGAATCCGGTGGTGACAGAGG |
| | | ATGTGGAAGGTGACAAGAAAATTTATAATGCTGAAGTTGCTGAATGGGATA |
| | | AGTTACGGCAACGATTGCTTGATGCCAGAAATAAAATCACCTCTGCTGAAT |
| | | CTGCGATAAATTCGGCGAGAAATAACGTCAGTGCCAGAACAAATGAACAA |
| | | AAGCATGCAAATGACGCTCTTAATGCCCTGTTGAAGGAAAAAGAGAATATC |
| | | CGTAGCCAGCTTGCTGACATCAATCAGAAAATAGCTGAAGAGAAAAGAAA |
| | | AAGGGATGAAATAAATATGGTAAAGGATGCCATAAAACTCACCTCTGATTT |
| | | CTACAGAACGATATATGATGAGTTCGGTAAACAAGCATCCGAACTTGCTAA |
| | | GGAGCTGGCTTCTGTATCTCAAGGGAAACAGATTAAGAGTGTGGATGATGC |
| | | ACTGAACGCTTTTGATAAATTCCGTAATAATCTGAACAAGAAATATAACAT |
| | | ACAAGATCGCATGGCCATTTCTAAAGCCCTGGAAGCTATTAATCAGGTCCA |
| | | TATGGCGGAGAATTTTAAGCTGTTCAGTAAGGCATTTGGTTTTACCGGAAAA |
| | | GTTATTGAACGTTATGATGTTGCTGTGGAGTTACAAAAGGCTGTAAAAACG |
| | | GACAACTGGCGTCCATTTTTTGTAAAACTTGAATCACTGGCAGCAGGAAGA |
| | | GCTGCTTCAGCAGTTACAGCATGGGCGTTTTCCGTCATGCTGGGAACCCCTG |
| | | TAGGTATTCTGGGTTTTGCAATTATTATGGCGGCTGTGAGTGCGCTTGTTAA |
| | | TGATAAGTTTATTGAGCAGGTCAATAAACTTATTGGTATCTGA |
| Colicin 10 | 162 | ATGGATAAAGTCACTGATAATTCTCCAGATGTGGAGAGCACAGAATCTACT |
| | | GAGGGGTCATTCCCAACTGTTGGGGTTGATACTGGCGATACGATTACAGCG |
| | | ACGCTTGCAACTGGAACTGAAAATGTTGGTGGAGGCGGTGGAGCATTTGGT |
| | | GGGGCCAGTGAAAGTTCTGCTGCGATACATGCAACCGCTAAATGGTCTACC |
| | | GCGCAGTTGAAAAAACATCAGGCTGAACAGGCTGCCCGTGCTGCTGCGGCT |
| | | GAGGCAGCATTGGCAAAAGCGAAATCTCAGCGTGATGCCCTGACTCAACGT |
| | | CTCAAGGATATTGTTAATGACGCTTTACGTGCTAATGCCGCTCGTAGTCCAT |
| | | CAGTAACTGACCTTGCTCATGCCAATAATATGGCAATGCAGGCAGAGGCTG |
| | | AGCGTTTGCGCCTTGCGAAGGCAGAGCAAAAAGCCCGTGAAGAAGCTGAA |
| | | GCAGCAGAAAAAGCGCTCCGGGAAGCAGAACGCCAACGTGATGAGATTGC |
| | | CCGCCAACAGGCTGAAACCGCGCATTTGTTAGCAATGGCGGAGGCAGCAGA |
| | | GGCTGAGAAAAATCGACAGGATTCTCTTGATGAAGAGCATCGGGCTGTGGA |
| | | AGTGGCAGAGAAGAAGCTGGCTGAGGCTAAAGCTGAACTGGCGAAGGCCG |
| | | AAAGCGATGTACAGAGTAAGCAAGCGATTGTTTCCAGAGTTGCAGGGGAGC |
| | | TTGAAAACGCTCAAAAAAGTGTTGATGTGAAGGTTACCGGATTTCCTGGAT |
| | | GGCGTGATGTTCAGAAAAAACTGGAGAGACAATTGCAGGATAAGAAGAAT |
| | | GAATATTCGTCAGTGACGAATGCTCTTAATTCTGCTGTTAGCATTAGAGATG |
| | | CTAAAAAAACAGAAGTTCAGAATGCTGAGATAAAATTAAAAGAAGCTAAG |
| | | GATGCTCTTGAGAAGAGTCAGGTAAAAGACTCTGTTGATACTATGGTTGGG |
| | | TTTTATCAATATATAACCGAACAATATGGGGAAAAATATTCCAGAATAGCT |
| | | CAGGATTTAGCTGAAAAGGCGAAGGGTAGTAAATTTAATAGTGTTGATGAA |
| | | GCACTTGCTGCATTTGAAAAGTATAAAAATGTACTGGATAAGAAATTCAGT |
| | | AAGGTTGATAGGGATGATATTTTTAATGCTTTAGAGTCTATTACTTATGATG |
| | | AGTGGGCCAAGCATCTAGAAAAGATCTCTAGGGCTCTTAAGGTTACTGGAT |
| | | ATTTGTCTTTCGGGTATGATGTATGGGATGGTACCCTAAAGGGATTAAAAAC |
| | | AGGAGACTGGAAGCCTTTATTTGTCACTCTGGAGAAGAGCGCGGTAGATTT |
| | | CGGCGTGGCAAAAATTGTGGCATTAATGTTTAGTTTTATTGTTGGTGCGCCT |
| | | CTTGGCTTCTGGGGAATTGCAATTATCACAGGTATTGTTTCTTCTTACATAG |
| | | GGGATGATGAGTTGAACAAGCTTAATGAATTACTAGGTATTTAA |
| Colicin K | 163 | ATGGCTAAAGAACTAAGTGGATATGGACCAACTGCTGGTGAGTCTATGGGG |
| | | GGAACAGGGGCGAATCTGAATCAGCAAGGTGGAAATAATAATAGTAATAG |
| | | TGGGGTTCACTGGGGTGGTGGTTCTGGGCATGGTAATAATGGTGGTCAGGG |
| | | AAATTCAAATTCATCTGGTTCTACATCAACTGTGATGAAAACCGGTGAGTCA |
| | | TATCTGACTCCGTGGGGTGATGTTGTCATTAATAATGATGGCCTTCCGGTGA |
| | | TGAATGGTATTGTTATGACAGAAGAGAATTCAACTCTGGTTGATAATCCATT |
| | | TGGAGGGGTCTCCAGAGTTCTTAATTCTTTAATTAGTGATATGCCATCACTT |
| | | TTTGCAGAAAGCTCAGGAAATAACAACAATAACACTGCTTCAGTTAATACT |
| | | GCACCAACGAATGCTCAGGTCAGTGATATGGATAAGAGTTCGAAGGTGGTG |
| | | AGTAATGTAATTAATGAAAAACAAAAGCAGAAGAATAAAATTGCTACACA |
| | | GATTAGTGAAAAACAGAAGAAAATCGAGGAGATGAAAAAAGTATTCAAGC |
| | | ATCATTCTTATCATGGTATCACTGATCTTGAGAGGGATGTTGATGAGTTGCA |
| | | AAAAAAATCAAATCAGCTTGATGCAGATATTAGCAAACTTAATTCTTATAA |
| | | AAATACATTGCAAAGTAAAATTGGTGATGTGAATAAACAGAAGGAAGCTG |
| | | AAGAAAAGGCTCGTGAAAATGCAGAGGTCGCTGAACATGAAACTCTTAATG |
| | | AAGAGAAACAGGCGGTGGCGGAAGCAGAGAAGCGTTTAGCTGAGGCGAAA |
| | | GCTGAACTGGCGAAGGCTGAAAGTGATGTACAGAGTAAGCAAGCTACTGTT |
| | | TCCAGAGTTGCAGGAGAGCTTGAAAACGCTCAGAAGAGTGTTGACGTGAAG |
| | | GTCACCGGATTTCCGGGATGGCGTGACGTTCAGAAAAAACTACAGCGTCAG |
| | | CTTGAAGCTAAGCAGGCTGAGTACTCCGCAGTTGAAAATGAACTTAAGAAT |
| | | GCTGTTAGTTTCAGGGATGGTAAGGCCGCTGAGGTTAAAGAGGCAGAGCAG |
| | | AAATTAAAAGAAGCTCAGGATGCTCTTGAAAAGAGCCAGATAAAAGATGCT |
| | | GTTGATACTATGGTTGGATTTTATCAATATATAACCGAACAATATGGAGAA |
| | | AAATACGCAAAAATTGCTCAGGATTTAGCGGAAAAGTCTAAAGGAAAGAA |
| | | AATTCAAGGTGTTGATGAAGCTCTGGCAGCATTTGAAAAGTATAAGAATGT |
| | | ACTGGATAAGAAATTCAGTAAGGTTGATCGGGATGCTATTTTTAATGCGTTA |
| | | GAGTCTGTTAATTATGATGAGTTATCTAAGAATTTAACGAAGATATCAAAGT |
| | | CGCTTAAAATCACCAGTCGAGTATCTTTTCTATATGATGTTGGTTCTGATTTT |
| | | AAAAATGCAATAGAAACTGGTAACTGGCGACCTTTGTTTGTCACGCTGGAG |
| | | AAAAGTGCGGTCGATGTAGGTGTGGCAAAAATTGTGGCATTAATGTTTAGT |
| | | TTTATTGTTGGTGTGCCTCTTGGTTTCTGGGGGATTGCAATTGTCACAGGTAT |
| | | TGTTTCTTCTTACATAGGGGATGATGAGTTGAGCAAACTTAATGAGTTGTTA |
| | | GGGATTTAA |
| Colicin U | 164 | ATGCCTGGATTTAATTATGGTGGACATGGTGACGGTACTGGCTGGAGTTCTG |
| | | AACGTGGTGATGGTCCGGCTCCGGGCGGTGGTATGCAGGGAAACGGTGGCG |
| | | GTCATAGCGGAAATAATGACAGTGGTAGCAATTCTGTGAGTCAGCAGATTT |
| | | CTGCCATTCAGAATGATCAGAAACTTAAACAGAAAGTGGTGAATATGCTGA |
| | | TTGCTGCGAGAAAAATGAATCCTGATGCGAAAATGATTCTGGGAAGTATTG |
| | | CTCCTTCTGGTGTTATGCAGGTCACCATTGAGGGGGTAACCTCCACGCAGGC |
| | | CAGGCAGCTTGGTCTGGGAGGGCTGGTCATGGGATATAACGCTTCAGGTGT |
| | | TATTGGGGCTGTGGGCGAAATAGACACCGGTCACCGCCTTAATGCATCAGG |
| | | AGCAAGTACTCCCGGAAGCGAAACCTCTGTGGACAGTTTTGTGAACGGACA |
| | | GAAACCCGCCGAAGAATGGCATGCTGTGGCGAAGGACAGCTGGACCGGGG |
| | | CTGGTCCGGTGAATACAGGGCTGGTTAATAACGCCATCAAAAGTGTCCGGA |
| | | TTATCAAAAAAGGCTATGTGACGGGCGTTCTCACGCCAGAAGAGGTGATGA |
| | | ATAAAGCAGAGTACAAAGCAATGCGACAGGCTTTTGATTCCCTTCCACTGG |
| | | CAAAACAGGGAGAGGCTGTCAGACAGATTGTCGCAGCCTGGAGTCTGGCAT |
| | | ATCAGGATTTTCCGGTAAATCTGAAAAAAGAGATGGGGCGTGTCACTGAAC |
| | | GGATTGTTGATGCTATTAACCTGGCGCTCATTCTTAATCAGACAGAGTCCCG |
| | | TTTGTCGGAATCACAAAAGAATGTTGATGTAGCGAACCAGATTATCAGTGA |
| | | TACAGTAAAAGCTATTAATGATGTAAATAAGAAAATAGCAGAAAAACGAA |
| | | ATCAGCAGGTATCACTGACTGATTTGATGAATAAAAAACAAAAAGAGGTCG |
| | | AAGATCTTAAAAAGATATTCAAAAATCACTCTTATCACCGTATTCGTGATGC |
| | | CCAGAGGGAATATGATGATGCCCGAAATAAATATGCTTTACTGGCCAGTGA |
| | | TATTAATGCGCTGCAGGCACAGGTTTCCGGACTGACGGCAAGGAAACAGCA |
| | | GGCAGAACAGAACAAGGCAGCAGCAGAAAAAGCAAAGGCAGATGCAGCG |
| | | GCGAAAGCTGCAGCAGAAAAAGCGGCTGCTGAAGCGAAAGCAAAGGCTGA |
| | | AGCTGAAAAAGCCAGAAAGGAAGCAGAGGAAAAAGCAAATGATGAGAAG |
| | | GCTGTTCTGACAAAAGCCAGTGAAATTATCATCAGTGTTGGTGATAAGGCT |
| | | GGGGAGTATCTGGGTGATAAATATAAAGTTCTTTCTCGTGAGATAGCAGAT |
| | | AATATCAAAAACTTTCAGGGCAAAACGATTCGTAGTTACGATGAGGCGATG |
| | | GCTTCTGTCAATAAACTGATGGCTAATCCTGATCTTAAAATTAATGCCGCCG |
| | | ACAGGGATGCAATTGTGAATGCCTGGAAAGCATTTGATGCAGAGGATATGG |
| | | GGAATAAGTTTGCCGCGCTGGGTAAGACGTTTAAAGCCGCAGATTATGTGA |
| | | TGAAGGCAAACAACGTCCGGGAGAAGAGTATCGAGGGGTATCAGACCGGA |
| | | AACTGGGGGCCGCTGATGCTGGAGATTGAGTCCTGGGTATTGAGTGGTATT |
| | | GCTTCTGCTGTGGCGCTATCATTTTTCTCAGCTATCTTTGGTACTTTTGCTAT |
| | | GCTGGGTGTTTTTTCGACTAGTTTAGCTGGGATCTTGGCTGTTATTTTGGCTG |
| | | GTTTAGTTGGGGCGTTGATTGATGATAATTTTGTTGATAAGTTAAATAATGA |
| | | AATTATTCGTCCTGCGTATTAA |
| Colicin Ia | 171 | ATGTCTGACCCTGTACGTATTACAAATCCCGGTGCAGAATCGCTGGGGTATG |
| | | ATTCAGATGGCCATGAAATTATGGCCGTTGATATTTATGTAAACCCTCCACG |
| | | TGTCGATGTCTTTCATGGTACCCCGCCTGCATGGAGTTCCTTCGGGAACAAA |
| | | ACCATCTGGGGCGGAAACGAGTGGGTTGATGATTCCCCAACCCGAAGTGAT |
| | | ATCGAAAAAAGGGACAAGGAAATCACAGCGTACAAAAACACGCTCAGCGC |
| | | GCAGCAGAAAGAGAATGAGAATAAGCGTACTGAAGCCGGAAAACGCCTCT |
| | | CTGCGGCGATTGCTGCAAGGGAAAAAGATGAAAACACACTGAAAACACTCC |
| | | GTGCCGGAAACGCAGATGCCGCTGATATTACACGACAGGAGTTCAGACTCC |
| | | TGCAGGCAGAGCTGAGAGAATACGGATTCCGTACTGAAATCGCCGGATATG |
| | | ACGCCCTCCGGCTGCATACAGAGAGCCGGATGCTGTTTGCTGATGCTGATTC |
| | | TCTTCGTATATCTCCCCGGGAGGCCAGGTCGTTAATCGAACAGGCTGAAAA |
| | | ACGGCAGAAGGATGCGCAGAACGCAGACAAGAAGGCCGCTGATATGCTTG |
| | | CTGAATACGAGCGCAGAAAAGGTATTCTGGACACCCGGTTGTCAGAGCTGG |
| | | AAAAAAATGGCGGGGCAGCCCTTGCCGTTCTTGATGCACAACAGGCCCGTC |
| | | TGCTCGGGCAGCAGACACGGAATGACAGGGCCATTTCAGAGGCCCGGAATA |
| | | AACTCAGTTCAGTGACGGAATCGCTTAACACGGCCCGTAATGCATTAACCA |
| | | GAGCTGAACAACAGCTGACGCAACAGAAAAACACGCCTGACGGCAAAACG |
| | | ATAGTTTCCCCTGAAAAATTCCCGGGGCGTTCATCAACAAATCATTCTATTG |
| | | TTGTGAGCGGTGATCCGAGATTTGCCGGTACGATAAAAATCACAACCAGCG |
| | | CAGTCATCGATAACCGTGCAAACCTGAATTATCTTCTGAGCCATTCCGGTCT |
| | | GGACTATAAACGCAATATTCTGAATGACCGGAATCCGGTGGTGACAGAGGA |
| | | TGTGGAAGGTGACAAGAAAATTTATAATGCTGAAGTTGCTGAATGGGATAA |
| | | GTTACGGCAAAGATTGCTTGATGCCAGAAATAAAATCACCTCTGCTGAATCT |
| | | GCGGTAAATTCGGCGAGAAATAACCTCAGTGCCAGAACAAATGAGCAAAA |
| | | GCATGCAAATGACGCTCTTAATGCCCTGTTGAAGGAAAAAGAGAATATACG |
| | | TAACCAGCTTTCCGGCATCAATCAGAAGATAGCGGAAGAGAAAAGAAAAC |
| | | AGGATGAACTGAAGGCAACGAAAGACGCAATTAATTTCACAACAGAGTTCC |
| | | TGAAATCAGTTTCAGAAAAATATGGTGCAAAAGCTGAGCAGTTAGCCAGAG |
| | | AGATGGCCGGGCAGGCTAAAGGGAAGAAAATACGTAATGTTGAAGAGGCA |
| | | TTAAAAACGTATGAAAAGTACCGGGCTGACATTAACAAAAAAATTAATGCA |
| | | AAAGATCGTGCAGCGATTGCCGCAGCCCTTGAGTCTGTGAAGCTGTCTGATA |
| | | TATCGTCTAATCTGAACAGATTCAGTCGGGGACTGGGATATGCAGGAAAAT |
| | | TTACAAGTCTTGCTGACTGGATCACTGAGTTTGGTAAGGCTGTCCGGACAGA |
| | | GAACTGGCGTCCTCTTTTTGTTAAAACAGAAACCATCATAGCAGGCAATGCC |
| | | GCAACGGCTCTTGTGGCACTGGTCTTCAGTATTCTTACCGGAAGCGCTTTAG |
| | | GCATTATCGGGTATGGTTTACTGATGGCTGTCACCGGTGCGCTGATTGATGA |
| | | ATCGCTTGTGGAAAAAGCGAATAAGTTCTGGGGTATTTAA |
| Colicin E1 | 172 | ATGGAAACCGCGGTAGCGTACTATAAAGATGGTGTTCCTTATGATGATAAG |
| | | GGACAGGTAATTATTACTCTTTTGAATGGTACTCCTGACGGGAGTGGCTCTG |
| | | GCGGCGGAGGTGGAAAAGGAGGCAGTAAAAGTGAAAGTTCTGCAGCTATTC |
| | | ATGCAACTGCTAAATGGTCTACTGCTCAATTAAAGAAAACACAGGCAGAGC |
| | | AGGCTGCCCGGGCAAAAGCTGCAGCGGAAGCACAGGCGAAAGCAAAGGCA |
| | | AACAGGGATGCGCTGACTCAGCGCCTGAAGGATATCGTGAATGAGGCTCTT |
| | | CGTCACAATGCCTCACGTACGCCTTCAGCAACAGAGCTTGCTCATGCTAATA |
| | | ATGCAGCTATGCAGGCGGAAGACGAGCGTTTGCGCCTTGCGAAAGCAGAAG |
| | | AAAAAGCCCGTAAAGAAGCGGAAGCAGCAGAAAAGGCTTTTCAGGAAGCA |
| | | GAACAACGACGTAAAGAGATTGAACGGGAGAAGGCTGAAACAGAACGCCA |
| | | GTTGAAACTGGCTGAAGCTGAAGAGAAACGACTGGCTGCATTGAGTGAAGA |
| | | AGCTAAAGCTGTTGAGATCGCCCAAAAAAAACTTTCTGCTGCACAATCTGA |
| | | AGTGGTGAAAATGGATGGAGAGATTAAGACTCTCAATTCTCGTTTAAGCTCC |
| | | AGTATCCATGCCCGTGATGCAGAAATGAAAACGCTCGCTGGAAAACGAAAT |
| | | GAACTGGCTCAGGCATCCGCTAAATATAAAGAACTGGATGAGCTGGTCAAA |
| | | AAACTATCACCAAGAGCCAATGATCCGCTTCAGAACCGTCCTTTTTTTGAAG |
| | | CAACCAGACGACGGGTTGGGGCCGGTAAGATTAGAGAAGAAAAACAAAAA |
| | | CAGGTAACAGCATCAGAAACACGTATTAACCGGATAAATGCTGATATAACT |
| | | CAGATCCAGAAGGCTATTTCTCAGGTCAGTAATAATCGTAATGCCGGTATCG |
| | | CTCGTGTTCATGAAGCTGAAGAAAATTTGAAAAAAGCACAGAATAATCTCC |
| | | TTAATTCACAGATTAAGGATGCTGTTGATGCAACAGTTAGCTTTTATCAAAC |
| | | GCTGACTGAAAAATATGGTGAAAAATATTCGAAAATGGCACAGGAACTTGC |
| | | TGATAAGTCTAAAGGTAAGAAAATCGGCAATGTGAATGAAGCTCTCGCTGC |
| | | TTTTGAAAAATACAAGGATGTTTTAAATAAGAAATTCAGCAAAGCCGATCG |
| | | TGATGCTATTTTTAATGCGTTGGCATCGGTGAAGTATGATGACTGGGCTAAA |
| | | CATTTAGATCAGTTTGCCAAGTACTTGAAGATTACGGGGCATGTTTCTTTTG |
| | | GATATGATGTGGTATCTGATATCCTAAAAATTAAGGATACAGGTGACTGGA |
| | | AGCCACTATTTCTTACATTAGAGAAGAAAGCTGCAGATGCAGGGGTGAGTT |
| | | ATGTTGTTGCTTTACTTTTTAGCTTGCTTGCTGGAACTACATTAGGTATTTGG |
| | | GGTATTGCTATTGTTACAGGAATTCTATGCTCCTATATTGATAAGAATAAAC |
| | | TTAATACTATAAATGAGGTGTTAGGGATTTAA |
| Colicin D | 173 | ATGAGTGATTACGAAGGTAGTGGTCCGACAGAAGGTATTGATTATGGGCAC |
| | | TCGATGGTCGTGTGGCCGTCAACAGGACTGATTTCGGGTGGTGATGTGAAA |
| | | CCAGGAGGCTCATCAGGTATCGCTCCATCCATGCCTCCGGGATGGGGGGAT |
| | | TACAGCCCACAGGGTATCGCACTTGTACAAAGTGTTCTTTTTCCGGGAATTA |
| | | TTCGCCGGATTATTCTGGATAAGGAACTTGAAGAGGGAGACTGGTCGGGAT |
| | | GGTCTGTCAGTGTGCATAGCCCTTGGGGAAACGAGAAAGTTTCCGCTGCAC |
| | | GAACAGTTCTTGAGAATGGTTTACGTGGTGGTTTGCCAGAACCGTCTCGCCC |
| | | GGCTGCTGTTTCTTTTGCCCGTCTGGAGCCTGCTTCCGGAAATGAGCAAAAA |
| | | ATTATTCGTCTTATGGTTACACAGCAACTGGAACAGGTAACGGATATTCCGG |
| | | CCAGCCAGTTACCAGCAGCGGGTAATAATGTACCGGTGAAATATCGTCTGA |
| | | TGGACCTTATGCAGAACGGTACGCAATATATGGCTATTATCGGAGGTATTCC |
| | | GATGACAGTGCCGGTAGTTGATGCCGTTCCAGTTCCGGACCGGAGTCGTCCG |
| | | GGAACAAATATTAAAGATGTTTACAGTGCCCCTGTATCACCAAATCTACCGG |
| | | ACCTGGTATTAAGTGTGGGTCAGATGAATACTCCGGTTCTGTCTAATCCCGA |
| | | AATTCAGGAAGAAGGAGTTATTGCTGAGACAGGTAATTATGTTGAGGCTGG |
| | | TTATACGATGTCCAGTAATAATCATGATGTCATTGTTCGTTTTCCTGAAGGC |
| | | AGTGATGTTTCTCCGTTATATATTTCAACTGTAGAGATTCTGGACAGTAATG |
| | | GTCTGAGTCAGCGTCAGGAAGCCGAAAATAAAGCAAAGGATGATTTCAGAG |
| | | TCAAGAAAGAAGAAGCGGTAGCCCGGGCTGAAGCTGAAAAAGCAAAAGCC |
| | | GAGTTATTTAGTAAAGCAGGTGTGAACCAGCCTCCTGTATATACACAGGAA |
| | | ATGATGGAAAGGGCTAATTCAGTAATGAATGAGCAGGGAGCTCTTGTTCTG |
| | | AATAACACTGCCAGTTCTGTACAACTGGCGATGACCGGAACTGGTGTCTGG |
| | | ACTGCTGCTGGTGATATTGCAGGCAACATAAGCAAGTTTTTCAGTAATGCTC |
| | | TGGAGAAAGTAACCATACCCGAAGTGAGTCCCCTGCTTATGCGGATTTCTCT |
| | | TGGTGCTCTGTGGTTTCATTCAGAAGAAGCTGGAGCAGGAAGTGATATCGT |
| | | GCCGGGGCGAAATCTGGAGGCGATGTTTTCGCTGAGTGCTCAGATGTTGGCT |
| | | GGACAGGGCGTGGTCATTGAACCTGGTGCGACGAGTGTAAATCTGCCTGTT |
| | | CGTGGACAATTGATAAACAGTAACGGGCAATTAGCTCTGGATTTACTGAAA |
| | | ACAGGGAATGAAAGTATCCCGGCTGCGGTTCCTGTTCTTAATGCTGTCCGTG |
| | | ATACAGCAACAGGACTGGATAAAATCACGTTACCAGCAGTAGTAGGCGCAC |
| | | CTTCCCGGACGATTCTGGTTAATCCGGTACCACAACCTTCGGTGCCAACAGA |
| | | TACAGGTAATCATCAACCCGTTCCGGTTACACCAGTGCATACAGGAACGGA |
| | | AGTAAAATCGGTCGAAATGCCAGTAACGACGATTACTCCTGTTTCTGATGTT |
| | | GGTGGACTACGAGATTTTATTTACTGGCGTCCTGATGCTGCTGGGACTGGTG |
| | | TTGAAGCTGTTTATGTGATGCTTAATGATCCTCTGGATTCAGGGCGATTTTC |
| | | GCGTAAACAACTTGATAAAAAATATAAACATGCTGGTGATTTTGGTATTAGT |
| | | GATACAAAAAAGAATCGTGAAACTCTTACTAAATTCAGGGATGCTATTGAG |
| | | GAGCATTTATCGGATAAGGATACAGTAGAGAAAGGAACATACCGAAGAGA |
| | | AAAAGGTTCAAAAGTTTATTTTAATCCTAATACGATGAATGTGGTTATAATT |
| | | AAGTCAAATGGTGAGTTCTTATCTGGGTGGAAAATAAATCCAGATGCGGAT |
| | | AATGGTCGAATTTATTTAGAGACAGGTGAACTATGA |
| Colicin E2 | 174 | ATGAGCGGTGGCGATGGACGCGGCCATAACACGGGCGCGCATAGCACAAGT |
| | | GGTAACATTAATGGTGGCCCGACCGGGCTTGGTGTAGGTGGTGGTGCTTCTG |
| | | ATGGCTCCGGATGGAGTTCGGAAAATAACCCGTGGGGTGGTGGTTCCGGTA |
| | | GCGGCATTCACTGGGGTGGTGGTTCCGGTCATGGTAATGGCGGGGGGAATG |
| | | GTAATTCCGGTGGTGGTTCGGGAACAGGCGGTAATCTGTCAGCAGTAGCTG |
| | | CGCCAGTGGCATTTGGTTTTCCGGCACTTTCCACTCCAGGAGCTGGCGGTCT |
| | | GGCGGTCAGTATTTCAGCGGGAGCATTATCGGCAGCTATTGCTGATATTATG |
| | | GCTGCCCTGAAAGGACCGTTTAAATTTGGTCTTTGGGGGGTGGCTTTATATG |
| | | GTGTATTGCCATCACAAATAGCGAAAGATGACCCCAATATGATGTCAAAGA |
| | | TTGTGACGTCATTACCCGCAGATGATATTACTGAATCACCTGTCAGTTCATT |
| | | ACCTCTCGATAAGGCAACAGTAAACGTAAATGTTCGTGTTGTTGATGATGTA |
| | | AAAGACGAACGACAGAATATTTCGGTTGTTTCAGGTGTTCCGATGAGTGTTC |
| | | CGGTGGTTGATGCAAAACCTACCGAACGTCCAGGTGTTTTTACGGCATCAAT |
| | | TCCAGGTGCACCTGTTCTGAATATTTCAGTTAATAACAGTACGCCAGAAGTA |
| | | CAGACATTAAGCCCAGGTGTTACAAATAATACTGATAAGGATGTTCGCCCG |
| | | GCAGGATTTACTCAGGGTGGTAATACCAGGGATGCAGTTATTCGATTCCCGA |
| | | AGGACAGCGGTCATAATGCCGTATATGTTTCAGTGAGTGATGTTCTTAGTCC |
| | | TGACCAGGTAAAACAACGTCAGGATGAAGAAAATCGCCGTCAGCAGGAAT |
| | | GGGATGCTACGCATCCGGTTGAAGCGGCTGAGCGAAATTATGAACGCGCGC |
| | | GTGCAGAGCTGAATCAGGCAAATGAAGATGTTGCCAGAAATCAGGAGCGAC |
| | | AGGCTAAAGCTGTTCAGGTTTATAATTCGCGTAAAAGCGAACTTGATGCAG |
| | | CGAATAAAACTCTTGCTGATGCAATAGCTGAAATAAAACAATTTAATCGATT |
| | | TGCCCATGACCCAATGGCTGGCGGTCACAGAATGTGGCAAATGGCCGGACT |
| | | TAAAGCTCAGCGGGCGCAGACGGATGTAAATAATAAGCAGGCTGCATTTGA |
| | | TGCTGCTGCAAAAGAGAAGTCAGATGCTGATGCTGCATTAAGTGCCGCGCA |
| | | GGAGCGCCGCAAACAGAAGGAAAATAAAGAAAAGGACGCTAAGGATAAAT |
| | | TAGATAAGGAGAGTAAACGGAATAAGCCAGGGAAGGCGACAGGTAAAGGT |
| | | AAACCAGTTGGTGATAAATGGCTGGATGATGCAGGTAAAGATTCAGGAGCG |
| | | CCAATTCCAGATCGCATTGCTGATAAGTTGCGTGATAAAGAATTTAAAAACT |
| | | TTGACGATTTCCGGAAGAAATTCTGGGAAGAAGTGTCAAAAGATCCCGATC |
| | | TTAGTAAGCAATTTAAAGGCAGTAATAAGACGAACATTCAAAAGGGAAAAG |
| | | CACCTTTTGCAAGGAAGAAAGACCAAGTAGGTGGTAGGGAACGCTTTGAAT |
| | | TACATCATGATAAACCAATCAGTCAGGATGGTGGTGTCTATGATATGAATA |
| | | ATATCAGAGTGACCACACCTAAGCGACATATTGATATTCATCGGGGTAAGT |
| | | AA |
| Colicin E3 | 175 | ATGAGCGGTGGCGATGGACGCGGCCATAACACGGGCGCGCATAGCACAAGT |
| | | GGTAACATTAATGGTGGCCCGACCGGGCTTGGTGTAGGTGGTGGTGCTTCTG |
| | | ATGGCTCCGGATGGAGTTCGGAAAATAACCCGTGGGGTGGTGGTTCCGGTA |
| | | GCGGCATTCACTGGGGTGGTGGTTCCGGTCATGGTAATGGCGGGGGGAATG |
| | | GTAATTCCGGTGGTGGTTCGGGAACAGGCGGTAATCTGTCAGCAGTAGCTG |
| | | CGCCAGTGGCATTTGGTTTTCCGGCACTTTCCACTCCAGGAGCTGGCGGTCT |
| | | GGCGGTCAGTATTTCAGCGGGAGCATTATCGGCAGCTATTGCTGATATTATG |
| | | GCTGCCCTGAAAGGACCGTTTAAATTTGGTCTTTGGGGGGTGGCTTTATATG |
| | | GTGTATTGCCATCACAAATAGCGAAAGATGACCCCAATATGATGTCAAAGA |
| | | TTGTGACGTCATTACCCGCAGATGATATTACTGAATCACCTGTCAGTTCATT |
| | | ACCTCTCGATAAGGCAACAGTAAACGTAAATGTTCGTGTTGTTGATGATGTA |
| | | AAAGACGAGCGACAGAATATTTCGGTTGTTTCAGGTGTTCCGATGAGTGTTC |
| | | CGGTGGTTGATGCAAAACCTACCGAACGTCCGGGTGTTTTTACGGCATCAAT |
| | | TCCAGGTGCACCTGTTCTGAATATTTCAGTTAATAACAGTACGCCAGCAGTA |
| | | CAGACATTAAGCCCAGGTGTTACAAATAATACTGATAAGGATGTTCGCCCG |
| | | GCAGGATTTACTCAGGGTGGTAATACCAGGGATGCAGTTATTCGATTCCCGA |
| | | AGGACAGCGGTCATAATGCCGTATATGTTTCAGTGAGTGATGTTCTTAGCCC |
| | | TGACCAGGTAAAACAACGTCAAGATGAAGAAAATCGCCGTCAGCAGGAAT |
| | | GGGATGCTACGCATCCGGTTGAAGCGGCTGAGCGAAATTATGAACGCGCGC |
| | | GTGCAGAGCTGAATCAGGCAAATGAAGATGTTGCCAGAAATCAGGAGCGAC |
| | | AGGCTAAAGCTGTTCAGGTTTATAATTCGCGTAAAAGCGAACTTGATGCAG |
| | | CGAATAAAACTCTTGCTGATGCAATAGCTGAAATAAAACAATTTAATCGATT |
| | | TGCCCATGACCCAATGGCTGGCGGTCACAGAATGTGGCAAATGGCCGGGCT |
| | | TAAAGCCCAGCGGGCGCAGACGGATGTAAATAATAAGCAGGCTGCATTTGA |
| | | TGCTGCTGCAAAAGAGAAGTCAGATGCTGATGCTGCATTGAGTTCTGCTATG |
| | | GAAAGCAGGAAGAAGAAAGAAGATAAGAAAAGGAGTGCTGAAAATAATTT |
| | | AAACGATGAAAAGAATAAGCCCAGAAAAGGTTTTAAAGATTACGGGCATG |
| | | ATTATCATCCAGCTCCGAAAACTGAGAATATTAAAGGGCTTGGTGATCTTAA |
| | | GCCTGGGATACCAAAAACACCAAAGCAGAATGGTGGTGGAAAACGCAAGC |
| | | GCTGGACTGGAGATAAAGGGCGTAAGATTTATGAGTGGGATTCTCAGCATG |
| | | GTGAGCTTGAGGGGTATCGTGCCAGTGATGGTCAGCATCTTGGCTCATTTGA |
| | | CCCTAAAACAGGCAATCAGTTGAAAGGTCCAGATCCGAAACGAAATATCAA |
| | | GAAATATCTTTGA |
| Colicin B | 176 | ATGAGTGATAATGAAGGTAGTGTACCGACAGAAGGTATTGATTACGGGGAC |
| | | ACAATGGTTGTGTGGCCGTCAACAGGACGAATTCCGGGCGGTGATGTGAAA |
| | | CCCGGAGGCTCATCAGGTCTCGCTCCATCCATGCCTCCGGGATGGGGGGATT |
| | | ACAGCCCACAAGGTATCGCACTTGTACAAAGTGTTCTTTTTCCTGGAATTAT |
| | | TCGCCGGATTATTCTTGATAAAGAACTTGAAGAGGGAGACTGGTCGGGATG |
| | | GTCTGTCAGTGTGCATAGCCCCTGGGGAAACGAGAAAGTTTCCGCTGCACG |
| | | AACAGTTCTTGAAAATGGTTTACGTGGTGGTTTGCCAGAACCGTCTCGCCCG |
| | | GCTGCTGTTTCTTTTGCCCGTCTGGAGCCTGCTTCCGGAAATGAGCAAAAAA |
| | | TTATTCGTCTTATGGTTACACAGCAACTGGAGCAGGTAACGGATATCCCTGC |
| | | CAGCCAGTTACCAGCAGCGGGTAATAATGTACCGGTAAAATATCGTCTGAC |
| | | GGACCTTATGCAGAATGGTACACAATATATGGCTATTATCGGAGGTATTCCG |
| | | ATGACAGTGCCAGTAGTGGATGCCGTTCCAGTTCCGGACCGGAGTCGTCCG |
| | | GGAACCAATATTAAAGATGTTTACAGTGCCCCTGTATCACCAAATCTACCGG |
| | | ACCTGGTATTAAGTGTGGGTCAGATGAATACTCCAGTTCGGTCTAATCCCGA |
| | | AATCCAGGAAGATGGCGTTATTTCTGAGACAGGGAATTATGTTGAGGCTGG |
| | | TTATACGATGTCCAGTAATAATCATGATGTCATTGTCCGTTTTCCTGAAGGC |
| | | AGTGGAGTTTCTCCGCTATATATTTCAGCCGTGGAGATTCTGGACAGTAATA |
| | | GTTTAAGCCAGCGCCAGGAAGCCGAAAATAACGCAAAGGATGACTTCAGAG |
| | | TCAAGAAAGAACAAGAAAATGACGAGAAGACGGTCCTGACAAAAACCAGC |
| | | GAGGTCATCATTAGTGTCGGTGACAAAGTCGGGGAATATCTTGGAGATAAA |
| | | TACAAGGCGCTTTCCCGTGAAATTGCAGAGAATATAAATAATTTTCAGGGA |
| | | AAAACGATTCGTAGTTATGATGATGCAATGTCTTCCATTAATAAGTTAATGG |
| | | CTAACCCCAGCCTTAAAATAAATGCAACGGACAAAGAAGCCATTGTGAATG |
| | | CGTGGAAAGCATTTAATGCTGAGGATATGGGGAATAAATTTGCTGCGTTGG |
| | | GTAAAACGTTCAAAGCAGCAGATTATGCAATAAAGGCAAACAACATCAGGG |
| | | AGAAGAGTATTGAGGGTTACCAGACTGGTAACTGGGGGCCATTAATGCTGG |
| | | AAGTCGAGTCCTGGGTTATCAGTGGGATGGCATCTGCTGTAGCTCTTAGTTT |
| | | GTTTTCTTTGACATTAGGCTCGGCCCTTATAGCCTTTGGTCTTTCGGCCACAG |
| | | TTGTTGGTTTTGTTGGCGTAGTTATTGCAGGTGCTATTGGTGCATTTATCGAT |
| | | GATAAATTTGTTGATGAGTTGAATCACAAGATCATAAAATAA |

TABLE 35
Example Colicin Protein Sequences

| | SEQ | |
|---|---|---|
| Protein | ID NO | Sequence (N-terminus to C-terminus) |
| Colicin Ib | 165 | MSDPVRITNPGAESLGYDSDGHEIMAVDIYVNPPRVDVFHGTPPAWSSFGNKTI |
| | | WGGNEWVDDSPTRSDIEKRDKEITAYKNTLSAQQKENENKRTEAGKRLSAAIA |
| | | AREKDENTLKTLRAGNADAADITRQEFRLLQAELREYGFRTEIAGYDALRLHTE |
| | | SRMLFADADSLRISPREARSLIEQAEKRQKDAQNADKKAADMLAEYERRKGIL |
| | | DTRLSELEKNGGAALAVLDAQQARLLGQQTRNDRAISEARNKLSSVTESLKTA |
| | | RNALTRAEQQLTQQKNTPDGKTIVSPEKFPGRSSTNHSIVVSGDPRFAGTIKITTS |
| | | AVIDNRANLNYLLTHSGLDYKRNILNDRNPVVTEDVEGDKKIYNAEVAEWDK |
| | | LRQRLLDARNKITSAESAINSARNNVSARTNEQKHANDALNALLKEKENIRSQL |
| | | ADINQKIAEEKRKRDEINMVKDAIKLTSDFYRTIYDEFGKQASELAKELASVSQ |
| | | GKQIKSVDDALNAFDKFRNNLNKKYNIQDRMAISKALEAINQVHMAENFKLFS |
| | | KAFGFTGKVIERYDVAVELQKAVKTDNWRPFFVKLESLAAGRAASAVTAWAF |
| | | SVMLGTPVGILGFAIIMAAVSALVNDKFIEQVNKLIGI* |
| Colicin 10 | 166 | MDKVTDNSPDVESTESTEGSFPTVGVDTGDTITATLATGTENVGGGGGAFGGA |
| | | SESSAAIHATAKWSTAQLKKHQAEQAARAAAAEAALAKAKSQRDALTQRLKD |
| | | IVNDALRANAARSPSVTDLAHANNMAMQAEAERLRLAKAEQKAREEAEAAEK |
| | | ALREAERQRDEIARQQAETAHLLAMAEAAEAEKNRQDSLDEEHRAVEVAEKK |
| | | LAEAKAELAKAESDVQSKQAIVSRVAGELENAQKSVDVKVTGFPGWRDVQKK |
| | | LERQLQDKKNEYSSVTNALNSAVSIRDAKKTEVQNAEIKLKEAKDALEKSQVK |
| | | DSVDTMVGFYQYITEQYGEKYSRIAQDLAEKAKGSKFNSVDEALAAFEKYKN |
| | | VLDKKFSKVDRDDIFNALESITYDEWAKHLEKISRALKVTGYLSFGYDVWDGT |
| | | LKGLKTGDWKPLFVTLEKSAVDFGVAKIVALMFSFIVGAPLGFWGIAIITGIVSS |
| | | YIGDDELNKLNELLGI* |
| Colicin K | 167 | MAKELSGYGPTAGESMGGTGANLNQQGGNNNSNSGVHWGGGSGHGNNGGQ |
| | | GNSNSSGSTSTVMKTGESYLTPWGDVVINNDGLPVMNGIVMTEENSTLVDNPF |
| | | GGVSRVLNSLISDMPSLFAESSGNNNNNTASVNTAPTNAQVSDMDKSSKVVSN |
| | | VINEKQKQKNKIATQISEKQKKIEEMKKVFKHHSYHGITDLERDVDELQKKSNQ |
| | | LDADISKLNSYKNTLQSKIGDVNKQKEAEEKARENAEVAEHETLNEEKQAVAE |
| | | AEKRLAEAKAELAKAESDVQSKQATVSRVAGELENAQKSVDVKVTGFPGWRD |
| | | VQKKLQRQLEAKQAEYSAVENELKNAVSFRDGKAAEVKEAEQKLKEAQDALE |
| | | KSQIKDAVDTMVGFYQYITEQYGEKYAKIAQDLAEKSKGKKIQGVDEALAAFE |
| | | KYKNVLDKKFSKVDRDAIFNALESVNYDELSKNLTKISKSLKITSRVSFLYDVG |
| | | SDFKNAIETGNWRPLFVTLEKSAVDVGVAKIVALMFSFIVGVPLGFWGIAIVTGI |
| | | VSSYIGDDELSKLNELLGI* |
| Colicin U | 168 | MPGFNYGGHGDGTGWSSERGDGPAPGGGMQGNGGGHSGNNDSGSNSVSQQI |
| | | SAIQNDQKLKQKVVNMLIAARKMNPDAKMILGSIAPSGVMQVTIEGVTSTQAR |
| | | QLGLGGLVMGYNASGVIGAVGEIDTGHRLNASGASTPGSETSVDSFVNGQKPA |
| | | EEWHAVAKDSWTGAGPVNTGLVNNAIKSVRIIKKGYVTGVLTPEEVMNKAEY |
| | | KAMRQAFDSLPLAKQGEAVRQIVAAWSLAYQDFPVNLKKEMGRVTERIVDAI |
| | | NLALILNQTESRLSESQKNVDVANQIISDTVKAINDVNKKIAEKRNQQVSLTDL |
| | | MNKKQKEVEDLKKIFKNHSYHRIRDAQREYDDARNKYALLASDINALQAQVS |
| | | GLTARKQQAEQNKAAAEKAKADAAAKAAAEKAAAEAKAKAEAEKARKEAE |
| | | EKANDEKAVLTKASEIIISVGDKAGEYLGDKYKVLSREIADNIKNFQGKTIRSYD |
| | | EAMASVNKLMANPDLKINAADRDAIVNAWKAFDAEDMGNKFAALGKTFKAA |
| | | DYVMKANNVREKSIEGYQTGNWGPLMLEIESWVLSGIASAVALSFFSAIFGTFA |
| | | MLGVFSTSLAGILAVILAGLVGALIDDNFVDKLNNEIIRPAY* |
| Colicin Ia | 177 | MSDPVRITNPGAESLGYDSDGHEIMAVDIYVNPPRVDVFHGTPPAWSSFGNKTI |
| | | WGGNEWVDDSPTRSDIEKRDKEITAYKNTLSAQQKENENKRTEAGKRLSAAIA |
| | | AREKDENTLKTLRAGNADAADITRQEFRLLQAELREYGFRTEIAGYDALRLHTE |
| | | SRMLFADADSLRISPREARSLIEQAEKRQKDAQNADKKAADMLAEYERRKGIL |
| | | DTRLSELEKNGGAALAVLDAQQARLLGQQTRNDRAISEARNKLSSVTESLNTA |
| | | RNALTRAEQQLTQQKNTPDGKTIVSPEKFPGRSSTNHSIVVSGDPRFAGTIKITTS |
| | | AVIDNRANLNYLLSHSGLDYKRNILNDRNPVVTEDVEGDKKIYNAEVAEWDK |
| | | LRQRLLDARNKITSAESAVNSARNNLSARTNEQKHANDALNALLKEKENIRNQ |
| | | LSGINQKIAEEKRKQDELKATKDAINFTTEFLKSVSEKYGAKAEQLAREMAGQ |
| | | AKGKKIRNVEEALKTYEKYRADINKKINAKDRAAIAAALESVKLSDISSNLNRF |
| | | SRGLGYAGKFTSLADWITEFGKAVRTENWRPLFVKTETIIAGNAATALVALVFS |
| | | ILTGSALGIIGYGLLMAVTGALIDESLVEKANKFWGI* |
| Colicin E1 | 178 | METAVAYYKDGVPYDDKGQVIITLLNGTPDGSGSGGGGGKGGSKSESSAAIHA |
| | | TAKWSTAQLKKTQAEQAARAKAAAEAQAKAKANRDALTQRLKDIVNEALRH |
| | | NASRTPSATELAHANNAAMQAEDERLRLAKAEEKARKEAEAAEKAFQEAEQR |
| | | RKEIEREKAETERQLKLAEAEEKRLAALSEEAKAVEIAQKKLSAAQSEVVKMD |
| | | GEIKTLNSRLSSSIHARDAEMKTLAGKRNELAQASAKYKELDELVKKLSPRAN |
| | | DPLQNRPFFEATRRRVGAGKIREEKQKQVTASETRINRINADITQIQKAISQVSN |
| | | NRNAGIARVHEAEENLKKAQNNLLNSQIKDAVDATVSFYQTLTEKYGEKYSK |
| | | MAQELADKSKGKKIGNVNEALAAFEKYKDVLNKKFSKADRDAIFNALASVKY |
| | | DDWAKHLDQFAKYLKITGHVSFGYDVVSDILKIKDTGDWKPLFLTLEKKAAD |
| | | AGVSYVVALLFSLLAGTTLGIWGIAIVTGILCSYIDKNKLNTINEVLGI* |
| Colicin D | 179 | MSDYEGSGPTEGIDYGHSMVVWPSTGLISGGDVKPGGSSGIAPSMPPGWGDYS |
| | | PQGIALVQSVLFPGIIRRIILDKELEEGDWSGWSVSVHSPWGNEKVSAARTVLEN |
| | | GLRGGLPEPSRPAAVSFARLEPASGNEQKIIRLMVTQQLEQVTDIPASQLPAAGN |
| | | NVPVKYRLMDLMQNGTQYMAIIGGIPMTVPVVDAVPVPDRSRPGTNIKDVYSA |
| | | PVSPNLPDLVLSVGQMNTPVLSNPEIQEEGVIAETGNYVEAGYTMSSNNHDVIV |
| | | RFPEGSDVSPLYISTVEILDSNGLSQRQEAENKAKDDFRVKKEEAVARAEAEKA |
| | | KAELFSKAGVNQPPVYTQEMMERANSVMNEQGALVLNNTASSVQLAMTGTG |
| | | VWTAAGDIAGNISKFFSNALEKVTIPEVSPLLMRISLGALWFHSEEAGAGSDIVP |
| | | GRNLEAMFSLSAQMLAGQGVVIEPGATSVNLPVRGQLINSNGQLALDLLKTGN |
| | | ESIPAAVPVLNAVRDTATGLDKITLPAVVGAPSRTILVNPVPQPSVPTDTGNHQP |
| | | VPVTPVHTGTEVKSVEMPVTTITPVSDVGGLRDFIYWRPDAAGTGVEAVYVML |
| | | NDPLDSGRFSRKQLDKKYKHAGDFGISDTKKNRETLTKFRDAIEEHLSDKDTVE |
| | | KGTYRREKGSKVYFNPNTMNVVIIKSNGEFLSGWKINPDADNGRIYLETGEL* |
| Colicin E2 | 180 | MSGGDGRGHNTGAHSTSGNINGGPTGLGVGGGASDGSGWSSENNPWGGGSGS |
| | | GIHWGGGSGHGNGGGNGNSGGGSGTGGNLSAVAAPVAFGFPALSTPGAGGLA |
| | | VSISAGALSAAIADIMAALKGPFKFGLWGVALYGVLPSQIAKDDPNMMSKIVTS |
| | | LPADDITESPVSSLPLDKATVNVNVRVVDDVKDERQNISVVSGVPMSVPVVDA |
| | | KPTERPGVFTASIPGAPVLNISVNNSTPEVQTLSPGVTNNTDKDVRPAGFTQGG |
| | | NTRDAVIRFPKDSGHNAVYVSVSDVLSPDQVKQRQDEENRRQQEWDATHPVE |
| | | AAERNYERARAELNQANEDVARNQERQAKAVQVYNSRKSELDAANKTLADAI |
| | | AEIKQFNRFAHDPMAGGHRMWQMAGLKAQRAQTDVNNKQAAFDAAAKEKS |
| | | DADAALSAAQERRKQKENKEKDAKDKLDKESKRNKPGKATGKGKPVGDKWL |
| | | DDAGKDSGAPIPDRIADKLRDKEFKNFDDFRKKFWEEVSKDPDLSKQFKGSNK |
| | | TNIQKGKAPFARKKDQVGGRERFELHHDKPISQDGGVYDMNNIRVTTPKRHIDI |
| | | HRGK* |
| Colicin E3 | 181 | MSGGDGRGHNTGAHSTSGNINGGPTGLGVGGGASDGSGWSSENNPWGGGSGS |
| | | GIHWGGGSGHGNGGGNGNSGGGSGTGGNLSAVAAPVAFGFPALSTPGAGGLA |
| | | VSISAGALSAAIADIMAALKGPFKFGLWGVALYGVLPSQIAKDDPNMMSKIVTS |
| | | LPADDITESPVSSLPLDKATVNVNVRVVDDVKDERQNISVVSGVPMSVPVVDA |
| | | KPTERPGVFTASIPGAPVLNISVNNSTPAVQTLSPGVTNNTDKDVRPAGFTQGG |
| | | NTRDAVIRFPKDSGHNAVYVSVSDVLSPDQVKQRQDEENRRQQEWDATHPVE |
| | | AAERNYERARAELNQANEDVARNQERQAKAVQVYNSRKSELDAANKTLADAI |
| | | AEIKQFNRFAHDPMAGGHRMWQMAGLKAQRAQTDVNNKQAAFDAAAKEKS |
| | | DADAALSSAMESRKKKEDKKRSAENNLNDEKNKPRKGFKDYGHDYHPAPKTE |
| | | NIKGLGDLKPGIPKTPKQNGGGKRKRWTGDKGRKIYEWDSQHGELEGYRASD |
| | | GQHLGSFDPKTGNQLKGPDPKRNIKKYL* |
| Colicin B | 182 | MSDNEGSVPTEGIDYGDTMVVWPSTGRIPGGDVKPGGSSGLAPSMPPGWGDYS |
| | | PQGIALVQSVLFPGIIRRIILDKELEEGDWSGWSVSVHSPWGNEKVSAARTVLEN |
| | | GLRGGLPEPSRPAAVSFARLEPASGNEQKIIRLMVTQQLEQVTDIPASQLPAAGN |
| | | NVPVKYRLTDLMQNGTQYMAIIGGIPMTVPVVDAVPVPDRSRPGTNIKDVYSA |
| | | PVSPNLPDLVLSVGQMNTPVRSNPEIQEDGVISETGNYVEAGYTMSSNNHDVIV |
| | | RFPEGSGVSPLYISAVEILDSNSLSQRQEAENNAKDDFRVKKEQENDEKTVLTK |
| | | TSEVIISVGDKVGEYLGDKYKALSREIAENINNFQGKTIRSYDDAMSSINKLMA |
| | | NPSLKINATDKEAIVNAWKAFNAEDMGNKFAALGKTFKAADYAIKANNIREKS |
| | | IEGYQTGNWGPLMLEVESWVISGMASAVALSLFSLTLGSALIAFGLSATVVGFV |
| | | GVVIAGAIGAFIDDKFVDELNHKIIK* |

### Example 24: Bacteriophages Engineered to Express Colicins Show Good Depth of Kill and Suppression of Regrowth by CFU Reduction Assay

The CFU reduction assay measures the ability of an individual bacteriophage or bacteriophage cocktail to deplete the bacteria present in a culture as well as to assess for the durability of this reduction. Efficacy is correlated with the magnitude of CFU depletion compared to the cells-only control.

Subset panels of 12-24 E. coli isolates were defined from a panel of 300 clinically relevant isolates based on their expected sensitivity to the wild-type parent bacteriophages of the colicin-engineered bacteriophages under study. These isolates were challenged with no phage, wild-type bacteriophages (p00ex, p004k, p00jc, p00ke, p00c0, p5516), or colicin-engineered bacteriophages (p00c0e105 (colK), p00kee072 (col10), p00kee154 (col1b), p00kee162 (colU)). The starting amount of bacteria was 1×10⁶ CFU. In samples where phage was added, the phage exposure was 1×10⁵ PFU for a multiplicity of infection of 0.1. The number of surviving CFU were assessed at 0, 4, and 24 hours after the initial exposure.

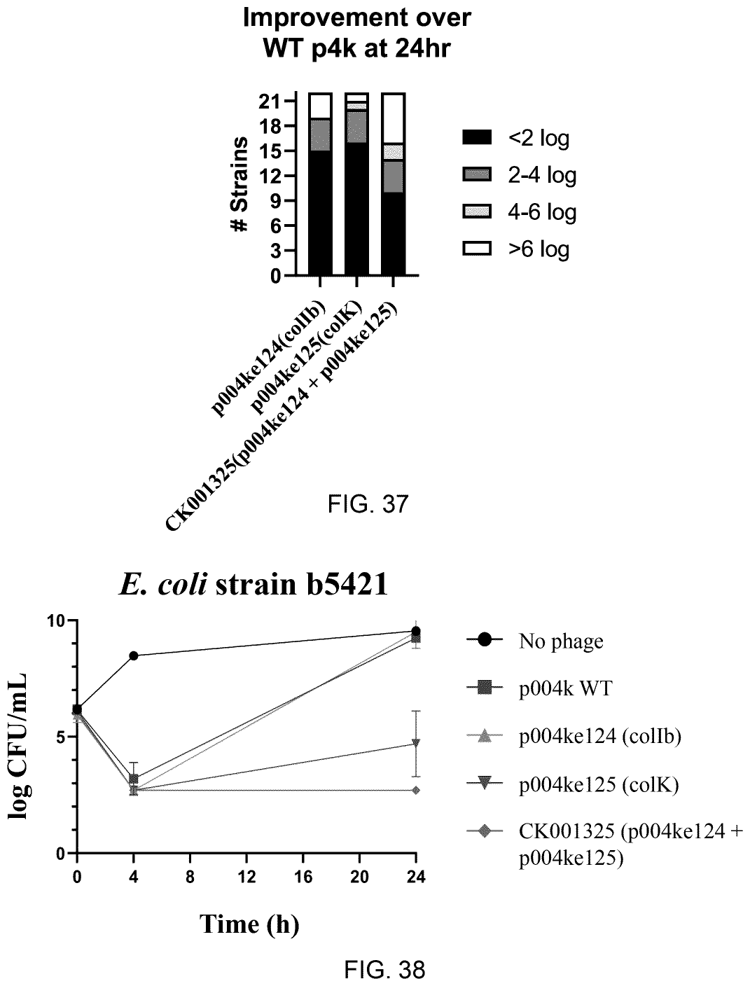

Maximum log CFU reductions compared to control (no bacteriophage) were assessed for bacteriophage p004k WT and p004k engineered with Colicin 1b (p004ke124 (col1b)), Colicin K (p004ke125 (colK)), or Colicin 1B+Colicin 10 (cocktail ck1325 (p004ke124 (col1b) and (p004ke125 (colK)) on a panel of 24 E. coli strains (FIG. 37). After 24 hours of treatment, all colicin-engineered p004k variants tested showed improvement compared to the wild-type p004k phage, with CK001325 showing the most improvement. As an example, combining p004ke124 (engineered with Colicin 1b) and p004ke125 (engineered with Colicin 10) into CK001325 showed improvement over either individual bacteriophage at 24 hours in E. coli b5421 (FIG. 38). Only strains that that were susceptible to wild type p004k are plotted.

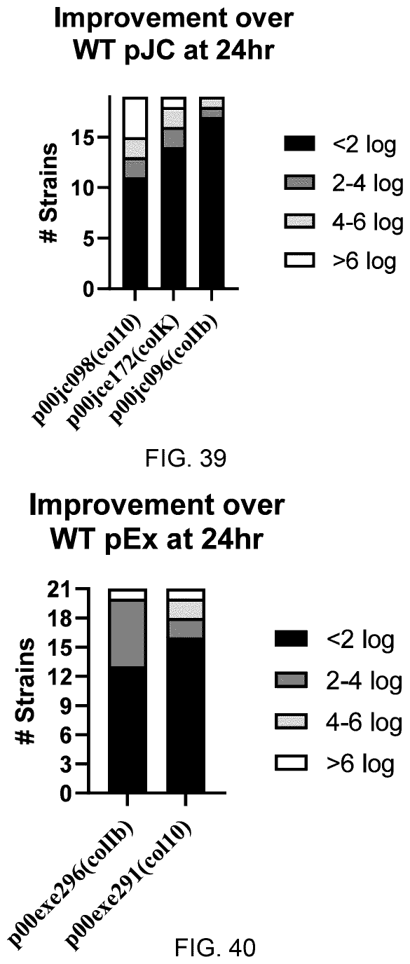

Maximum log CFU reductions compared to control (no bacteriophage) were assessed for bacteriophage p00jc WT and p00jc engineered with Colicin 10 (p00jce098 (col10)), Colicin K (p00jce172 (colK)), or Colicin 1B (p00jce096 (col1b)) on a panel of 24 E. coli strains (FIG. 39). After 24 hours of treatment, all colicin-engineered p00jc variants tested showed improvement compared to the wild-type p00jc phage. Log CFU reductions were also assessed on a second strain panel (n=12) for p00jc engineered with Colicin 10 (p00jce098 (col10)), Colicin U (p00jce175 (colU)), or Colicin K (p00jce172 (colK)) (FIG. 40). After 24 hours of treatment, both p00jce098 and p00jce172 showed improvement compared to the wild-type p00jc phage. Only strains that that were susceptible to wild type p00jc are plotted.

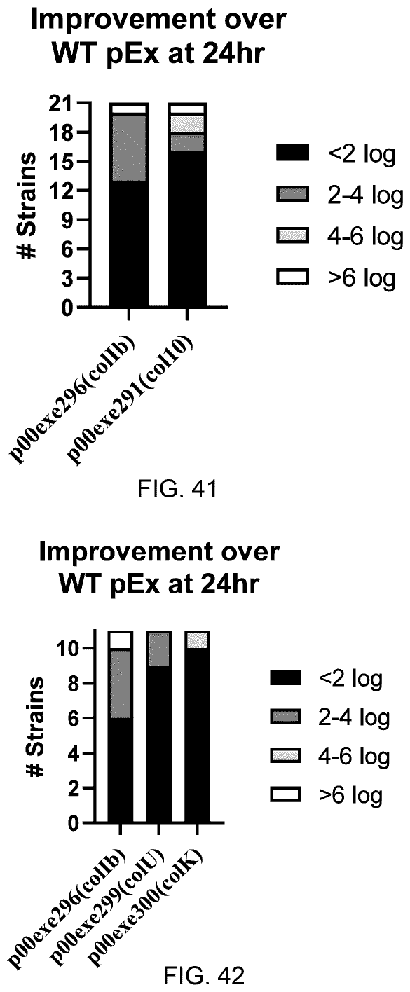

Maximum log CFU reductions compared to control (no bacteriophage) were assessed for p00ex WT and p00ex variants engineered with Colicin 1b (p00exe296 (col1b)) or Colicin 10 (p00exe291 (col10)) on a panel of 24 E. coli strains (FIG. 41). After 24 hours of treatment, both colicin-engineered p00ex variants tested showed improvement compared to the wild-type p00ex phage. Log CFU reductions were also assessed on a second strain panel (n=12) for p00ex engineered with Colicin 1b (p00exe296 (col1b)), Colicin U (p00exe299 (colU)), or Colicin K (p00exe300 (colK)) (FIG. 42). After 24 hours of treatment, all colicin-engineered p00ex variants tested showed improvement compared to the wild-type p00jc phage. Only strains that that were susceptible to wild type p00ex are plotted.

### Example 26: Cocktail CFU Reduction Assays

Colicin-engineered bacteriophage cocktails were tested against 217 strains at MOI of 1 in urine, of which 69 strains were tested with 5+ replicates and the remainder tested with 2 replicates (Table 36) Testing was performed by adding phage cocktail to bacterial cells, removing samples after 0, 4, and 24 h of incubation, and enumerating surviving CFUs by dilution and spotting.

TABLE 36
Example Colicin-Engineered Bacteriophage Cocktails

| phage | CK000618 | CK001369 | CK001360 | CK001391 | CK001352 | CK001372 | CK001390 |
|---|---|---|---|---|---|---|---|
| p00ex | FC | colicin U | FC | colicin U | FC | colicin U | colicin U |
| | (p00exe014) | (p00exe299) | (p00exe014) | (p00exe299) | (p00exe014) | (p00exe299) | (p00exe299) |
| | | | | | | | colicin 10 |
| | | | | | | | (p00exe291) |
| | | | | | | | colicin Ib |
| | | | | | | | (p00exe296) |
| | | | | | | | colicin K |
| | | | | | | | (p00exe300) |
| p004k | FC | colicin K | FC | colicin K | FC | colicin K | colicin K |
| | (p004ke009) | (p004ke127) | (p004ke009) | (p004ke127) | (p004ke009) | (p004ke127) | (p004ke127) |
| | | | | | | | colicin Ib |
| | | | | | | | (p004ke124) |
| p00c0 | FC | FC | FC | FC | N/A | N/A | N/A |
| | (p00c0e103) | (p00c0e103) | (p00c0e103) | (p00c0e103) | | | |
| p00ke | WT | colicin 10 | WT | colicin 10 | WT | colicin 10 | colicin 10 |
| | (p00ke) | (p00kee072) | (p00ke) | (p00kee072) | (p00ke) | (p00kee072) | (p00kee072) |
| p00jc | WT | colicin 10 | WT | colicin 10 | WT | colicin 10 | colicin 10 |
| | (p00jc) | (p00jce098) | (p00jc) | (p00jce098) | (p00jc) | (p00jce098) | (p00jce098) |
| | | | | | | | colicin K |
| | | | | | | | (p00jce172) |
| p5516 | WI | WT | WT | WT | WT | WT | WT |
| | (p5516) | (p5516) | (p5516) | (p5516) | (p5516) | (p5516) | (p5516) |
| p6984 | N/A | N/A | N/A | N/A | WT | WT | WT |
| | | | | | (p6984) | (p6984) | (p6984) |
| p6921 | N/A | N/A | WT | WT | N/A | N/A | N/A |
| | | | (p6921) | (p6921) | | | |
| p6977 | N/A | N/A | N/A | N/A | WT | WI | WT |
| | | | | | (p6977) | (p6977) | (p6977) |
| Liquid HR | 70.2 | 76.2 | n/a | n/a | 76.6 | 70.2 | 76.2 |
| (urine) 0.75 | | | | | | | |
| AUC % | | | | | | | |
| (n = 356) | | | | | | | |
| Liquid HR | 45.6 | 49.8 | n/a | n/a | 48.6 | 45.6 | 49.8 |
| (urine) Delta | | | | | | | |
| Endpoint | | | | | | | |
| OD > 0.25 | | | | | | | |

Throughout the Examples and specification, “CK000618” is referred to interchangeably as “CK618” and “CK000618”; “CK001369” is referred to interchangeably as “CK1369” and “CK001369 Colicin”; “CK001360” is referred to interchangeably as “CK1360” and “CK001360 WT”; “CK001391” is referred to interchangeably as “CK1391” and “CK001391 Colicin”; “CK001324” is referred to interchangeably as “CK1324” and “CK001324 WT”; “CK001352” is referred to interchangeably as “CK1352” and “CK001352 WT”; “CK001372” is referred to interchangeably as “CK1372” and “CK001372 Colicin”; and “CK001390” is referred to interchangeably as “CK1390” and “CK001390 Colicin-Max.”

Replicates with Log CFU Reduction

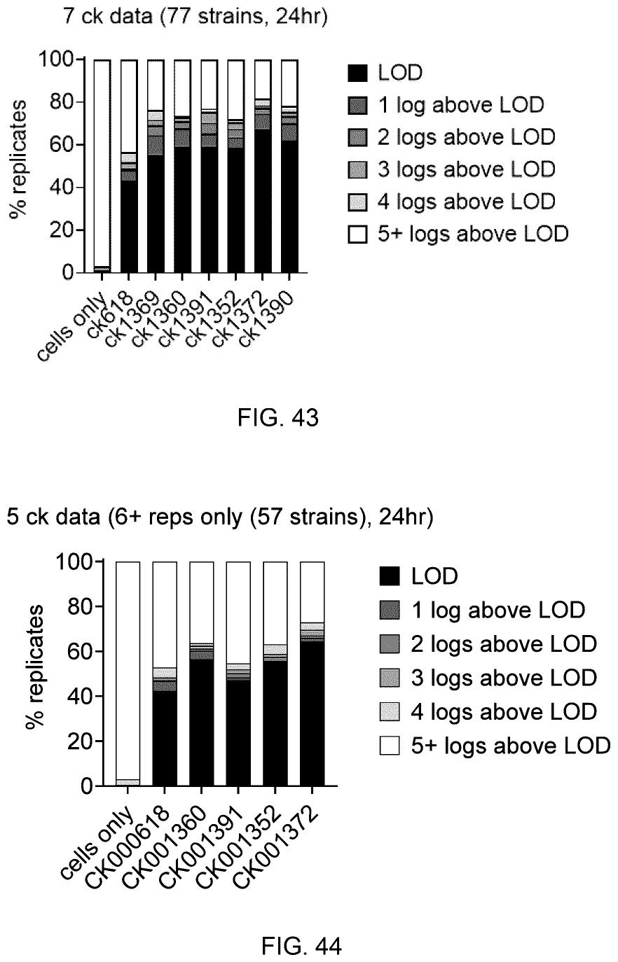

When assessing between replicates, CK001372 and CK001360 showed improved CFU reduction at 24 h compared to cocktails CK000618, CK001391, and CK001352 (FIGS. 56-57). Data was analyzed by calculating the number of replicates at LOD and if not at LOD, how many logs above LOD each replicate was. LOD was typically a 4-6 log reduction from cells only. The first 77 strains were run in duplicate (FIG. 43). CK001369 showed improved CFU reduction at 24 h compared to CK000618 and CK001372 showed improved CFU reduction at 24 h compared to CK001352. The 57 strain (6+ replicates) dataset (FIG. 44) showed improved performance for CK001360 and CK001372 at 24 h compared to cocktails CK000618, CK001391, and CK001352. CK001372 performed slightly better than CK001360.

### CFU Reduction Time Points

Among a comparison of CFU Reduction time points between cocktails tested, CK001372 showed the most improved total number of strains compared to CK000618, and net improvement compared to CK618 after 24 h (Table 37).

TABLE 37
Example Comparisons of CFU Reduction Time Points

| | CK001352 | CK001372 | CK001360 | CK001391 | CK001390 | CK001369 |
|---|---|---|---|---|---|---|
| Type of Difference | WT | Col | WT | Col | ColMax | Col |
| Improved over | 30 | 49 | 41 | 47 | 21 | 20 |
| CK000618* | | | | | | |
| Worse than | 9 | 7 | 5 | 12 | 3 | 0 |
| CK000618^{†} | | | | | | |
| Net Improvement | 21 | 42 | 36 | 35 | 18 | 20 |
| Percent Improved | 77% | 88% | 89% | 80% | 88% | 100% |

| | | |
|---|---|---|
| Number of strains | 216 | 77 |
| evaluated | | |

*>2 log improvement over CK000618 at 24 h
^{†}Underperforms CK000618 by >2log at 24 h

CFU reduction of 1e6 PFU/mL and 1e6 CFU/mL were run in 400 ul final volume and 3.3 log(CFU/mL) was LOD. Six replicates of each baseline isolate combined with cocktail were averaged. CFU reduction efficacy was present for all isolates at 4 hours on average but rebound rates were high at 24 hours

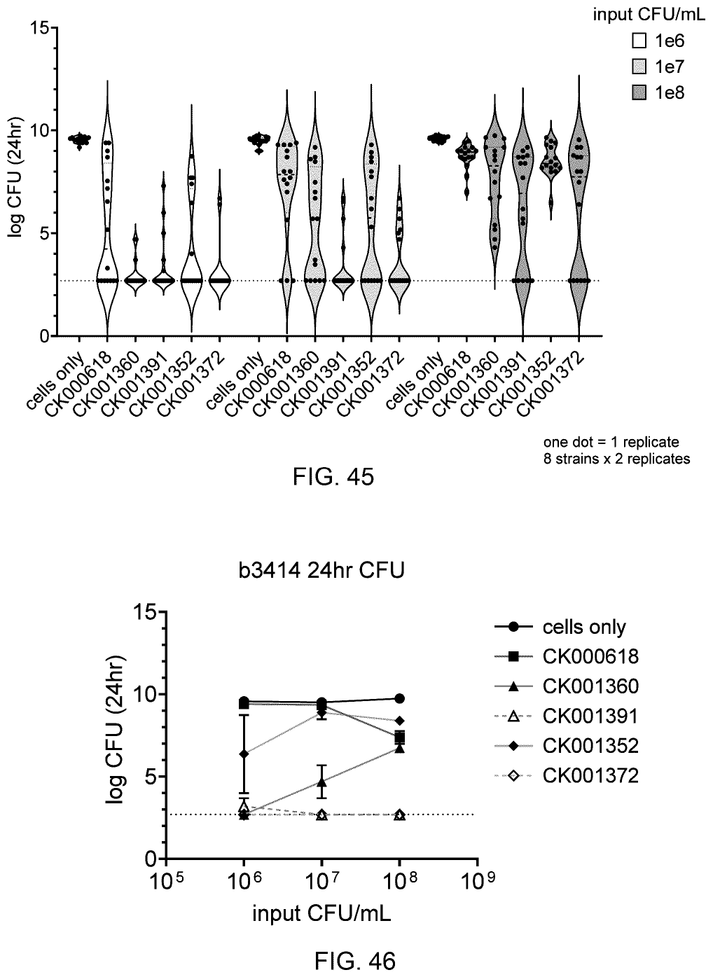

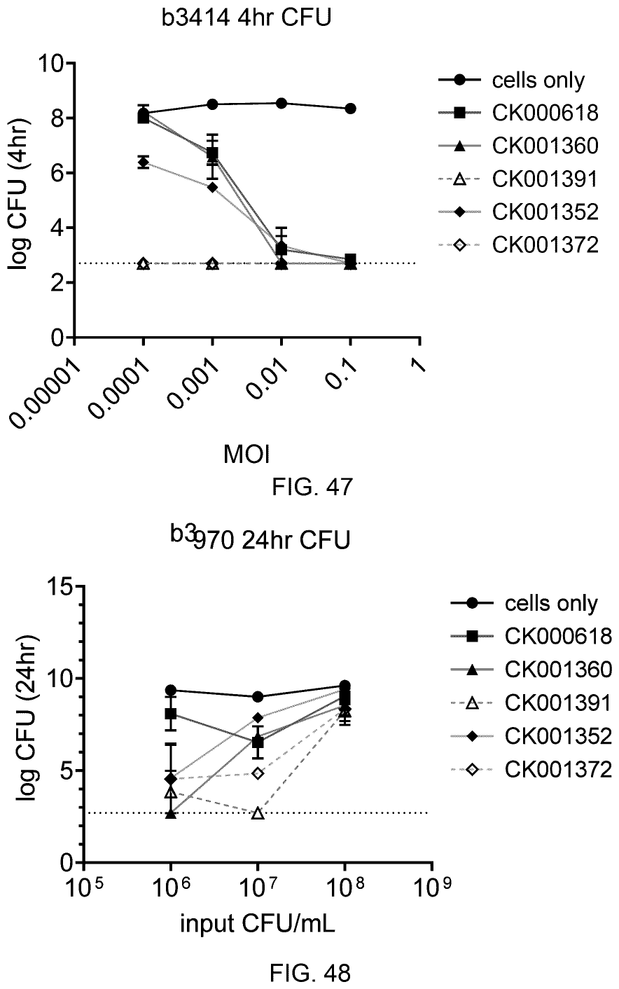

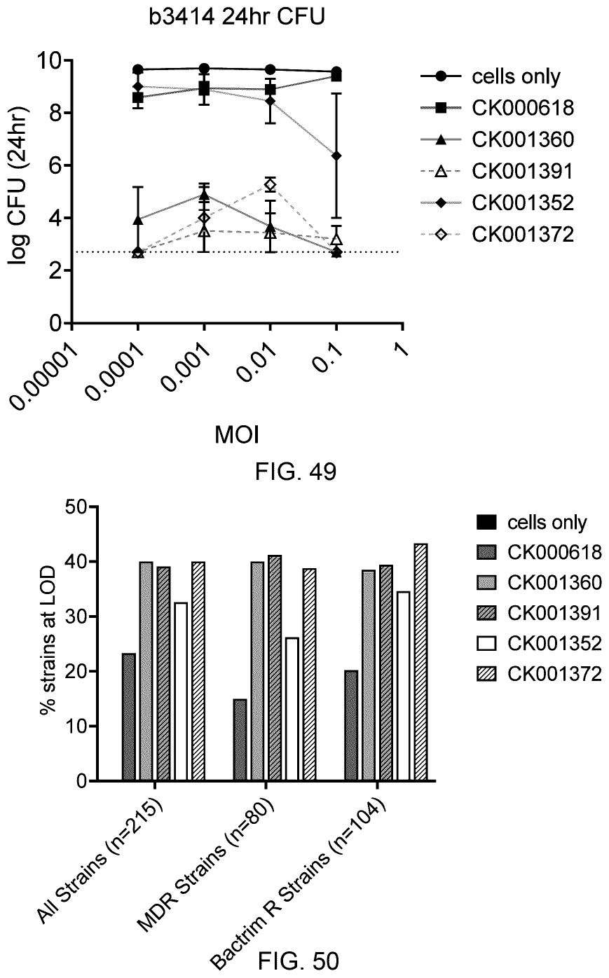

A range of input bacterial titers and input MOIs (representative of different possible levels of colonization or phage delivery in an infection setting) were tested to determine impact on WT and colicin-containing cocktail performance. The experiments testing a range of input CFUs utilized 1e8, 1e7, or 1e6 CFU/mL at MOI 0.1. The experiments testing a range of MOIs utilized an input CFU of 1e6 CFU/mL and MOIs of 0.1, 0.01, 0.001, or 0.0001. Cells and phage cocktail were mixed in pooled human urine supplemented with 0.5% casamino acids and incubated at 37 C with shaking for 24 h. Each treatment was prepared in duplicate. Samples were removed 4 and 24 h after mixing and the number of surviving CFUs was enumerated. Among the cocktails tested, increased input CFU showed more improvement (e.g., increased rate of killing) of the colicin engineered cocktails compared to their respective WT versions in the b3414 strain and the b3970 strain (FIGS. 45, 48). For example, CK001372 showed an improvement compared to CK001352, and CK1391 showed an improvement compared to CK1360 (FIG. 46). Among the cocktails tested, colicin-containing cocktails were found to kill faster at low MOI relative to their respective versions not engineered to contain colicins (FIG. 47). For example, CK001372 killed faster at low MOI compared to CK001352, and CK1391 killed faster at low MOI compared to CK1360. At 4 hours after infection, CK618, CK1360, CK1391, CK1352, and CK1372 all decreased the concentration of the b414 bacteria with increasing MOI, and a MOI of 0.1 resulted in a concentration below the LOD for all cocktails at 4 hours (FIG. 47). Furthermore, colicin-containing cocktails were found to provide better suppression of cell growth after 24 h at low MOI relative to their respective versions not engineered to contain colicins (FIG. 49). For example, CK001372 provides better suppression than CK001352 at all MOIs tested, and CK001391 provides better suppression than CK001360 at MOIs of 0.0001 and 0.001.

Cocktail CFU reduction summary on 217 strains shows colicin improvement of 7% for CK1372 over WT cocktail CK1352 at strains at LOD, CK1360 has comparable activity. Cocktails were tested against 217 strains at MOI of 1 in urine, with 69 strains tested with 5+ replicates and the remainder tested with 2 replicates

### Example 26: CFU Reduction by Metadata

Phage cocktails and bacteria were mixed in pooled human urine with 0.5% casamino acids at titers of 1e6 PFU/mL and 1eC CFU/mL, respectively, for an MOI of 1. Each treatment was prepared in duplicate. Samples were removed 4 and 24 h after mixing and the number of surviving CFUs was enumerated. At the 24 hour timepoint, cocktails containing additional wild-type phages (CK001360 and CK001352) performed 11-25% better on MDR strains and 15-19% better on BactrimR strains than CK000618. CK001360 and its colicin-engineered counterpart, CK001391, performed similarly. Colicin-containing cocktail CK001372 performed 13% better on MDR strains and 8% better on BactrimR strains than its non-colicin counterpart CK001352 CK001360 and CK001372 had comparable activity (FIG. 50 and Table 38). Among the cocktails tested, CK000618 showed the largest difference in BactrimR strains. Among the cocktails tested, no cocktails had a significant bias towards hitting MDR strains. CK000618 missed MDR strains more often than other cocktails tested. CK001360 and CK001391 hit MDR strains just as often as they missed them. CK001352 and CK001390 missed >5% more MDR strains. Colicin cocktail CK1372 appeared to have remedied this discrepancy

TABLE 38
Percentage of strains at LOD in MDR and BactrimR
strains at 24 hours (5+ log reduction)

| | All Strains | MDR Strains | Bactrim R Strains |
|---|---|---|---|
| Cocktails | (n = 215) | (n = 80) | (n = 104) |
| blank | 0 | 0 | 0 |
| CK000618 | 23 | 15 | 20 |
| CK001360 | 40 | 40 | 39 |
| CK001391 | 39 | 41 | 39 |
| CK001352 | 33 | 26 | 35 |
| CK001372 | 40 | 39 | 43 |

Hit threshold was LOD at 24 h for the CFU reduction assay (2.5e3 CFU/ml) which was equivalent to >4-log reduction when compared against the cells only control. Table 39 depicts CFU reduction data cut by location for states with at least 10 strains that were not suppressed at 24 hour timepoint. Strains were considered not suppressed if the raw CFU/mL value was greater than 7 (Log 10) at the 24 hour timepoint. For each cocktail calculated the percentage of strains from a particular state that had rebound >7 CFU/mL log 10. For most cocktails tested, clinical isolates of strains from California showed the greatest number of rebound. Strains from Illinois or Michigan showed the greatest efficacy of the tested cocktails by this measure.

TABLE 39
CFU reduction data cut by location

| State | All Strains | CK000618 | CK001360 | CK001391 | CK001352 | CK001372 |
|---|---|---|---|---|---|---|
| Code | Number | Percent | | | | |
| AL | 12 | 50 | 33 | 33 | 33 | 42 |
| CA | 38 | 66 | 45 | 47 | 61 | 47 |
| FL | 35 | 49 | 37 | 40 | 43 | 40 |
| IL | 11 | 9 | 0 | 9 | 0 | 9 |
| NY | 21 | 52 | 38 | 38 | 43 | 38 |
| TX | 15 | 27 | 20 | 20 | 13 | 13 |

### Example 28: Efficacy of Colicin 10 & U in a Neutropenic Thigh Model (Study 23-END-056)

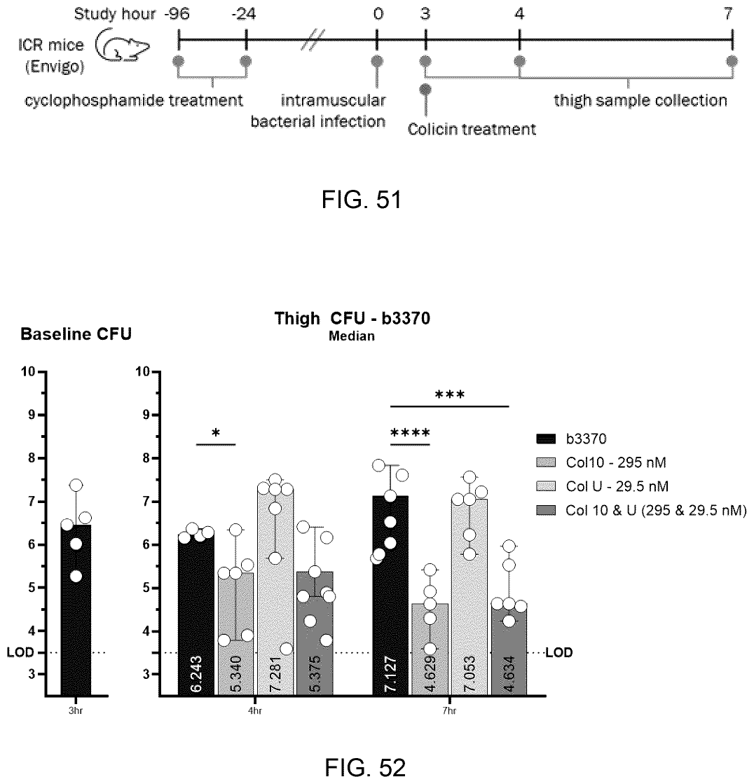

Study 23-END-056 was performed to evaluate the statistical difference between Control and Colicin treatment and sustained reduction of bacteria burden to LOD with colicin treatment in an ICR mouse model (FIG. 51). Specifically, the efficacy of 29.5 nM Colicin U, and combination therapy of both of 295 nM Colicin 10 & 29.5 nM Colicin U were evaluated in an ICR mouse model compared with no treatment (Table 40 and FIG. 52). In these experiments, due to high endotoxin, Colicin U was diluted 1:10 more than Colicin 10. At the 4 h and 7 h time points, treatment with 295 nM Colicin 10 lowered CFU, whereas no change was seen after treatment with 29.5 Colicin U.

TABLE 40
Thigh Efficacy Mouse Model

| | | | | | Harvest | | |
|---|---|---|---|---|---|---|---|
| Study | Induction of | Bacterial | | Total | time | Route of | |
| group | neutropenia | infection | Treatment | volume | (h) | administration | Total n |
| 1 | Cyclo- | E. coli | None (0.1 ml Tris- | N/A | 4, 7 | Intramuscular | 10 |
| | phosphamide | strain | buffered saline, | | | | |
| | (first dose | b3370 | vehicle) | | | | |
| 2 | 150 mg/kg; | 0.1 ml, 10⁷ | Colicin 10 (0.1 mL) | 1.5 mL | 4, 7 | Intramuscular | 10 |
| 3 | second dose | CFU/ml) | Colicin U (0.1 mL) | 1.5 mL | 4, 7 | Intramuscular | 10 |
| 4 | 100 mg/kg) | | Colicin 10 & U | 1.5 mL | 4, 7 | Intramuscular | 15 |
| | | | (0.1 mL) | | | | |

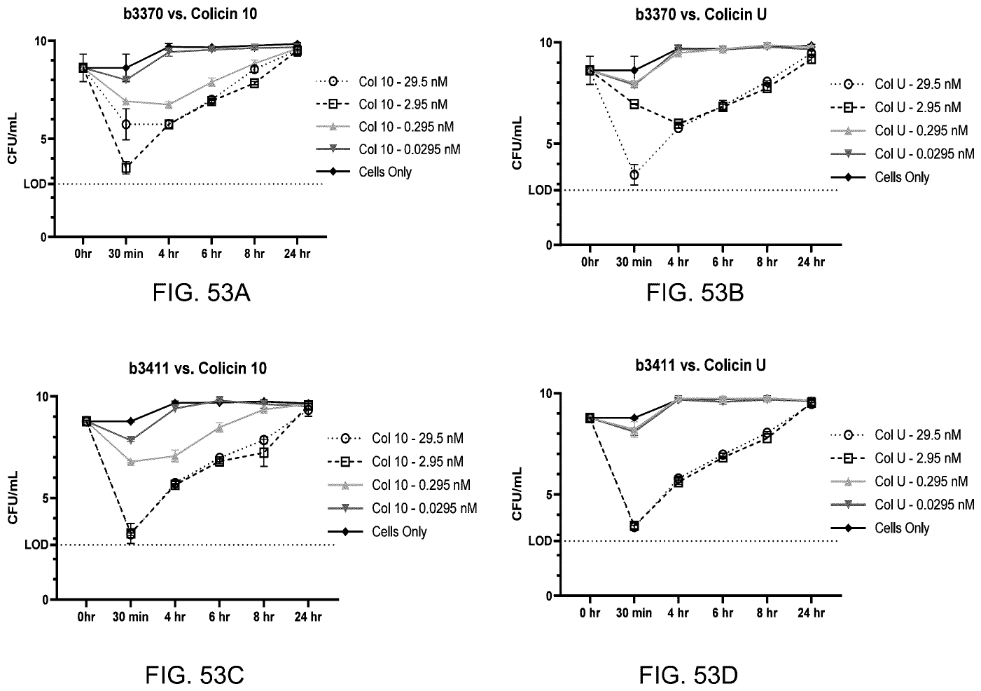

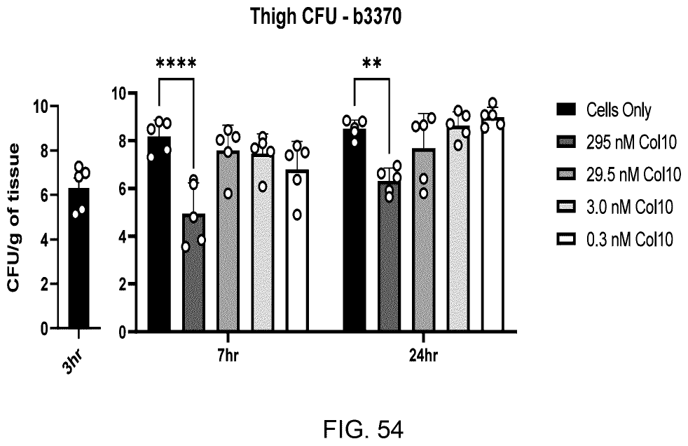

To compare Colicin activity in CFU reduction to effects seen in vivo, CFU reduction was observed in b3411 and b3370 after adding colicins Col10 and ColU (FIGS. 53A-53D and 54). Colicins produced an immediate significant reduction of CFU/mL (˜30 min) after adding colicins before bacterial regrowth. Bacteria demonstrated steady increase in CFU/mL in all timepoints post 30 min.

While preferred embodiments of the present disclosures have been shown and described herein, it will be obvious to those skilled in the art that such embodiments are provided by way of example only. Numerous variations, changes, and substitutions will now occur to those skilled in the art without departing from the disclosures. It should be understood that various alternatives to the embodiments of the disclosures described herein may be employed in practicing the disclosures. It is intended that the following claims define the scope of the disclosures and that methods and structures within the scope of these claims and their equivalents be covered thereby.

Additional Sequences

SEQ ID NO 183:
TATTACGAGATACACGTCCGAACCAACGCCATCCATTACTAATTGCACGAGAACCAGTGTGCCATG
TTTGCCAATCAAACTGAAGAAGAGTACGGTCTGGAGCTTCCCATTTCTCTAGTTCACCTTTTTCAAT
TTTTTCGAGGACTTCTTTGTGCCACTGGCGATAGATAAGTTCGCCATCAGGAATCTGACTGAACTCT
GCAGTTCCTGTTGCAAAATGAGTAGGACACACATCAGCGTTAACAAGACCAAGAATATGTTCAGAA
TGGTAACGAGGGTTATCATAGTCAGGTTGTCCTGCAGTAATAAAGTGTTGGCCGACTGGAATATCA
GGGCGAGGAACATCGTCATGGTGATATCCAGGAATAGCAGGATACCAACCAGGCATCAGCATGTG
GACTCGTGAATCAAATACAACATGTTGATTAGTCCAATCAATTGGGAGATTGGCAATAAAGCTACG
AGTAATAGGACCACCATTATCCCAAGCAAAACTCAAATCACAGTTAAAGAACATTGGTTCATTTTT
GATTTGGTCATTTTGTACGTTATGAGCAAAACCACCTACTGCACGAGCTTGACTATTAAACACTTTA
GAACTATTCATATTATTTTCCTCGACATTCTTTAATGAAATTTTCTAGATTGGTAGATTCAATAGCTT
TTTTACGAGCTTCTGCGTGTTCTTCTTCAGACAATTTACATTTATAATTAACCCAGACGTTATGGTCA
AAACTAGTCGTGGCATATAATAACAATGCTCCAACGAATAGCAAAAACGGCGATAAGAATACAAA
ACCAGGAACACCATCCATATCGTAAGCCTTTTGTAATACTGCAACAGAAAGAGCTACGTAAAAAAG
TGATACAAATATAGTTAAAAGACCAATTCGGTTGGTTTCTGTTAATTTCATAATATTCTCCTCATGTT
TTAATAGGACCACTATAACATAGTCCTATAATTGTGTAAACAGTTTTGTGAAGTTATTTGAAAGGTT
TTACCATATGGAAATTTTGTTTGAAACGTTTGACATAATCGTCGTGTCCTAAGCTTTGGTACATACT
TACAACTTTGCTGTAGTTTTCCCAGGCGTACTCACGGAACCAACCGGAGGTGATGCTTGTGCAGAC
TTTTTCCAATGCCATCATAAAACTGGCTTCAGGGTTAATTTCAAAGTTATTAGGAATCTGAGAACGT
TCTAATGCTAGTACACATGATTCTTCGTATACACCGGCCAATTTGACCATTTCTGGTAATGATTCGA
ACTTCTCGCGTGAGGTCATGACTTCAGAACCATCTTTCATATAAAACGTATAAGCCGGACGGTCTGT
TAGTGCAATGGCTTCGTGAATAGTATCATGGTCATACGTATAGATGTCATCTTTAAAGAATGCGCTT
TTAGAAGTGTCAAGAACTGGGTGAGCATAGTTCAAGGTTTCTTTCTGGCGTTTCAGCATAATTTCAG
TAAGTTCATCGTTCAGTGTAACACCTTTACCACGAAGATACTGAATATGGTTCATAGTCTTTAAAAA
GAATGGATTATTTTTCTTGAAACGATGAGACATCTTAATTGCAAGACACATCTCAGGAGTTGCCCA
GTAAAACCCGTTCAAACGGTCTTTAGTCAAATTTTTCTTGGCGTATTTTAAAAGGTCATAAGAAGAC
GTGAAATCTTCAGGAGTGAGTTCACCGACTAATTCACGGACATTAGTGATTGCCGGAACAATGTAT
GCTTCAAAATGAGTTTCTCGTCCATTATGGAAGCATTTGAAAACTTGGACACCAGGGTTATCTTGGT
GAACATATACACCCATCATTTGATTTTTGAATACATTCCAAGAACCTTGGTCCGCAATAAAGTCCCA
ATCACTATTTTTGATAGCGCTAGAATCAATAAGGCCGTGATAGTGAAGTGCACGAGAACCAATAAC
TAACATCATAATAATTTCCTCGAATTTAGATTACTTAAATTTCGCGACATGCGATATTATATTCATC
CCAGAGGTCATCGTGTGAAGCCTCTACCAATGGAACGATTGTACCGTTTATGTCTTCATTATAAGGA
CAAATTGTACAGTGCGCATCTTCGAATCGGATGCCTTTGTGTTCAGATTTTTTATAACGCATATCGA
ATATCTTTGCAAACTTTTCACGACTAATAGTTTTAGCAACTACTTTGGTTCCGATACTTTCGTAATAA
ATCATTGTGGACTCCATTTAATTTGATAGGACTACTATAACATAGCCCTATCTTGTTGTAAACTGTTT
TAGCAAGAATAACTAGATGATACCCAGCCTTGAACAGTGATACTATTGTGGGATAATGAGATCGCA
TTCTGGAGATTCAGACCTGCGTTATCTTCAATCCATAATGTAACTTCTTCGCGTTCTTCTGTTGCAAT
TTGGAACAGGTTTTCCAGAGCAGCTACTGCCGTACGTACAGCACAATCTAATTCACGAGACATATT
GGTTCCTTAGCAAGTGTGAGAAGATGCAACCCAACCACCATCTTCAAGGCTCAAGTTGTAGTCGTA
TTGGTAAACGTCGGCCCATTCAGCATAACCGTTTTTTGTACAACGTTCAAGATCGGCTTTAAGATGT
CCGGGTGAATGATAGGTTCCACCCATGCCATAAGCTGGATACAAGTTGAATGTTGTGTTTTGTTTAT
TAGCAATTTCTTTGGCAGCCTCAATAGCTGCATAGATTTTTGCTACAGCTTCAGCCAGTTCGGTTTG
TTCTTTAGACATAATGATTCCTTAGCACAGCGCAGAAGAGGAAACCCAAGCACCTTCAGTAATAAC
GTAACCATTACCTTCACGCTTAACATCGCCACCATTATTTTCAATTTCATTTTTCATCCACTGGGATA
CGATTTCACCCTTAGGATAATAAGTACGACCGCTACCATAATCACCTACAGAAAACGATACTGCAT
ATTGGTCTGCGATCTTTGTTGCTTCTTCTTCGATACGTTCAGCTTGATTCAGAAGTTTATAAATTGCA
TCTGAAGCAGATTCGGCATCATTAAACTCTGGTACTTTAATTTCAATAATCTTACTCATTTTCTACAT
TCCTTAATAAAGTTTTCGATTGTTTCATTTTCACGTCTAGCTAGGAGTTCATTATAACTAATCATTTG
AGAAGCGTAAACTTTTTTATTTTTCTTGTCATGGTTTATAAAATCGTAGTAAAAATAGACTACAGTA
CAAAATGATGCGATGGATGCAAAGGCTTGGGTATACCGTAAGTCCTGCCATTGATCTGTGAATCCA
CCAGTGTTCCATATAAATCCTAACACAACGCCGTCAATAATAAATCCAACTAATCCAAAGGCAAAT
CCCATGCCAATACTCATAATAAAAATTAGAATGTTCTCACCAAGAGAAGGCCATACGGTTAAGAGT
TGAGGCTTTTGCATCTTATTTCCTTAACGAATTCCATAGCTTCTTCATATTCATACTTTTTAACAGCT
TCATCATACTGAGCTTGGGCTTTTGCGCACTGAATTTTCCATTCTCTTAATCGGGCTCTGTGATGAC
GTCCTTGGTACCAATATCCCACCCAGTTAATAGGCATTAATAAAAACGGTACAATAAGTGGTGCGA
CGAACATTAATGAAAATGCTGCATCAGACGATATAACTTCACTAGAATCTAAAACGGCTCCTATAA
TCATAAAGATTAAAAATGCTAAAGCTAAAATAGGGCCAAATAGTACTTCATTTGGGATTAATTTAC
GCTTGTACTCATACGCCCAGGGCTTACTTGGCATGTATAGGGTTGGCTTTGACATATTCTCTACATT
CCATAATAAATTGCTCAAGGATTAAATTGTTAGTGAAATTAGGACTAACAACCATAGTTTCATAAA
GCGCATCAATAGCACTTAAAGCATCAACTCTTTTAAGCAAAGGCTTGCCTTCATACATAGTTTCAGA
ATCGATGATTAACGTAAAGTAATATTTTTTATTATCGACTAGAACTTCTGTATCGATATAAAACATA
TTAGACCTCAGAAGCTTATCTCTAAAACCATAATAATCTTCAGAGCGTTCGCTGATATATTCCAATT
CAGTGTTTAAACAACAAACAGACCACGTAGGAGCAACGAAATCAGTGAATCTAATATCATGGTCTG
CAACTAGTTTAGCAATAGCTCCGACATCATTAATCATGATACCTGGGGCTAATAAAGATTTTCGGTT
ATTTGCATATTCAGTTGTCTCATAAGAAGCTGACCAGTACCATTTACGCCCGGTTGCAACATATCGT
AATGAATCAACTAAAGACGTAGGTGGATTTACCTTATCTCGGAACTCTTCGAACATTGAGTCAATA
TCATTATTTTGGTTTCGCATGTTAACTCCTTTATTATCCAAAAAGGAGGACCGGAGTCCTCCGTATT
ATTTTATTTCAAAGAGTTAATATATTCTTGTACATCAGCAGAAGTGGTTTCAACACCGACTGGAGTG
CCATTGAATGTTTCAACACGAGTCAGAGTGTCTTCGATATCAACCTTAGTCAGTGCAGCAATTTCGA
TTACATCATCAGCCGTAGAGATACCAAGAGCATTGGCTGCTCGAGTTTCGCGGATGTATTCAAGTTT
GATTGCCAGATTCTGACGAGCATCATCAAGTTCAACCACCTTACGAGCAATTTCAATTCGCATAGC
GGCATAACCGTCAGCTTTTGTATTAAGTTGTTCGGCAGTTCGACGATACAGAAGACCAAGCTTAGC
ATGCATGGTAACATCTTGTCCTTCAGCCAGGAGCTTACGAATTTCACGTTCCTTAGAAGCGGCTTGC
TTGTTCTTTTCATTAACAAGTTCGCGAATACGTTTTTCTTCATTAATAGATTTAACAGAAGCCGTTTT
TAGTTCTTTGATTTGGGTAATCAACTTATCTGCAGCTGCGTTATACTGTTCTTCGACAGTCAGATTTT
TAGCCATAGCAGTACCCAGTTTAGTGCGGATGAATTCAACAAGTTTCTTCAGTGTGTTCATATTATT
ACCTTTCAATTGGTGGTTTATTATCCAACGAGAGCATTATACTCTGCTCTCAAGAGTTTGTAAATTA
TTTTTTAATCAAATTGACATAAAACTTGGCGTCTTCAGGGTGCATGCCTTCACAGTATTCTTCAACC
ATGAACTTCTTAACAGCTTCATAAGGACCGCTCAGATTAAGCTCGATTGTCCAAAACTTGGAATCTT
GGACTGAATCGATACGAACTTCAGGGTGACGGTTGCGAATAACTTCTTCAGTGTATTCATAATCAA
CGACATCAATACTAACTGTAGCCATTTTGTTTCTCCTCTGTAGTTGATAAGTCTATAGTATCACCAT
CCTTGGTGTTTGTACATCATTATTTTTAATTTTTCCAACTTAACAACTTGTAGTGTCCTTCATTCGTA
CGAGGTTTATCCCAAGAAATACTAATACATGATTCGCTGTCAATAGAAGTCGCCGGGTTTTTCTCTA
CAGTAAGACCTTCGCTTTCCAGCCATTCAATAGTATTTGCGCATAAATCTGAAGAACACCTCATCGG
AAACTGGCAAGATTTTTCACCTTTAGATGCAGCGTTATACATCTTTTCTAAAATACGCTTTTGCACG
TTTTTAAAAATAGTTTCAGCAGATTCTTCAGATTTTTTATTCAGTTCAGTATAAAGACTCATAATATT
CTCCTCATTAATAGGGCTCATAATATCTCAATCATAAGCCCATGTACGCATTTATTTCATATTATCG
AAATATTCTTCAGCGATTTCGACATTATCATGGTAAACTTTAGAAGACAGTTTAACATAACCTTCTG
CTGCGAACATATTAATCACAACCTTTACAGTATACCATTGTCCGTCTTCATTACCCATTACTGCGTA
AGTTTCAAACATCGGATGGTCAGGTCCAATAACTTTAATATCATTCACCGTACGTCCGAAATCTTCT
GGAACGCATTTCATAAAGAAGTTGAACAGTTCACCGTAATTATCCATTTCATTCTCCAAGGTTTTTC
TGTATCGGTAGTTGATAGTTGTATAGTACCACGGAAGAACAGGGATGTAAACAGTTTTGTGAAAAA
AATTTTTAAAAAGTTTTGTGGAATTCTAGGGCAGGGAGGGGAAATCAAAGGATAGGATAATATATT
ATAAAGGGCAGAAACTAAATGATGCCTAGAGAGGTCGGGAAAGGCCTAGATACCAAAAAGCCCTA
TCATTTAGATAGGGCTTTAAAATTATTTACCTAGTTTAGTTATTATAGATTCGGCAGCAGCTTTTAG
CTTAGATGCAGTGTTAATACGATTTTTAATTTCAGTATACGCATCGCCAAGAACATCAAGGCTATCA
GAATAAACACTTACACTATTGCGTTCGCTATTTGAAATAGAACCTTTAGTTTTACGATCAAAATCAG
TGTACACGTCTTGAAGATCACGGGCTGCCTTCTTTGCATCATCCAATGCATCAAGAGCTTTATTCAA
AGCATTGACGTATTTTTTAGTGTCAAACATATTCTTAGAAATTTTAACAGGTGCACGTTCAGGAGTA
GCTAAGGTGGATTTAGGAACCTGTGTGCCAG

What is claimed is:

1. A recombinant bacteriophage comprising a nucleic acid sequence encoding a Colicin 10, Colicin Ib, Colicin U, or Colicin K.

2. The recombinant bacteriophage of claim 1, comprising the Colicin 10.

3. The recombinant bacteriophage of claim 2, wherein the Colicin 10 has at least 80% identity to SEQ ID NO 166.

4. The recombinant bacteriophage of claim 2, wherein the Colicin 10 is a homolog of the Colicin 10 having SEQ ID NO: 166.

5. The recombinant bacteriophage of any one of claims 1-4, comprising the Colicin Ib.

6. The recombinant bacteriophage of claim 5, wherein the Colicin 1b has at least 80% identity to SEQ ID NO 165.

7. The recombinant bacteriophage of claim 5, wherein the Colicin 1b is a homolog of the Colicin 1b having SEQ ID NO: 165.

8. The recombinant bacteriophage of any one of claims 1-7, comprising the Colicin U.

9. The recombinant bacteriophage of claim 8, wherein the Colicin U has at least 80% identity to SEQ ID NO 168.

10. The recombinant bacteriophage of claim 8, wherein the Colicin U is a homolog of the Colicin U having SEQ ID NO: 168.

11. The recombinant bacteriophage of any one of claims 1-10, comprising the Colicin K.

12. The recombinant bacteriophage of claim 11, wherein the Colicin K has at least 80% identity to SEQ ID NO 167.

13. The recombinant bacteriophage of claim 11, wherein the Colicin K is a homolog of the Colicin K having SEQ ID NO: 167.

14. The recombinant bacteriophage of any one of claims 1-13, wherein the recombinant bacteriophage binds to and/or infects Escherichia.

15. The recombinant bacteriophage of any one of claims 1-14, wherein the recombinant bacteriophage is a recombinant Tequatrovirus.

16. The recombinant bacteriophage of claim 15, wherein the recombinant Tequatrovirus is a recombinant p004k.

17. The recombinant bacteriophage of any one of claims 1-16, wherein the recombinant bacteriophage has at least 80% sequence identity with p004k (ATCC Accession No. PTA-127149).

18. The recombinant bacteriophage of claim 15, wherein the recombinant Tequatrovirus is a recombinant p00ex.

19. The recombinant bacteriophage of any one of claims 1-14 or 18, wherein the recombinant bacteriophage virus has at least 80% sequence identity with p00ex (ATCC Accession No. PTA-127145).

20. The recombinant bacteriophage of any one of claims 1-14, wherein the recombinant bacteriophage is a recombinant Mosigvirus.

21. The recombinant bacteriophage of claim 20, wherein the recombinant Mosigvirus is a recombinant p00c0.

22. The recombinant bacteriophage of any one of claims 1-14 or 20-21, wherein the recombinant bacteriophage virus has at least 80% sequence identity with p00c0 (ATCC Accession No. PTA-127143).

23. The recombinant bacteriophage of any one of claims 1-14, wherein the recombinant bacteriophage is a recombinant Phapecoctavirus.

24. The recombinant bacteriophage of claim 23, wherein the recombinant Phapecoctavirus is a recombinant p00jc.

25. The recombinant bacteriophage of any one of claims 1-14 or 23-24, wherein the recombinant bacteriophage virus has at least 80% sequence identity with p00jc (ATCC Accession No. PTA-127147).

26. The recombinant bacteriophage of any one of claims 1-14, wherein the recombinant bacteriophage is a recombinant Myoviridae.

27. The recombinant bacteriophage of claim, wherein the recombinant Myoviridae is a recombinant p00ke.

28. The recombinant bacteriophage of any one of claims 1-14 or 26-27, wherein the recombinant bacteriophage virus has at least 80% sequence identity with p00ke (ATCC Accession No. PTA-127148).

29. A recombinant Tequatrovirus bacteriophage comprising a nucleic acid sequence encoding Colicin U.

30. The recombinant Tequatrovirus bacteriophage of claim 29, wherein the nucleic acid encoding Colicin U is at least 90% identical to SEQ ID NO: 164, and the recombinant Tequatrovirus bacteriophage is at least 90% identical to p00ex (ATCC Accession No. PTA-127145).

31. A recombinant Tequatrovirus bacteriophage comprising a nucleic acid sequence encoding Colicin K.

32. The recombinant Tequatrovirus bacteriophage of claim 31, wherein the nucleic acid encoding Colicin K is at least 90% identical to SEQ ID NO: 163, and the recombinant Tequatrovirus bacteriophage is at least 90% identical to p004k (ATCC Accession No. PTA-127149).

33. A recombinant Phapecoctavirus bacteriophage comprising a nucleic acid sequence encoding Colicin 10.

34. The recombinant Tequatrovirus bacteriophage of claim 33, wherein the nucleic acid encoding Colicin 10 is at least 90% identical to SEQ ID NO: 162, and the recombinant Phapecoctavirus bacteriophage is at least 90% identical to p00jc (ATCC Accession No. PTA-127147).

35. A recombinant Myoviridae bacteriophage comprising a nucleic acid sequence encoding Colicin 10.

36. The recombinant Myoviridae bacteriophage of claim 35, wherein the nucleic acid encoding Colicin 10 is at least 90% identical to SEQ ID NO: 161, and the recombinant Myoviridae bacteriophage is at least 90% identical to p00ke (ATCC Accession No. PTA-127148).

37. The recombinant bacteriophage of any one of claims 1-36, wherein the nucleic acid sequence is operably linked to a promoter sequence.

38. The recombinant bacteriophage of claim 37, wherein the promoter sequence is at least about 80% to SEQ ID NO: 1-11, 19, 64 or 65.

39. The recombinant bacteriophage of any one of claims 1-38, wherein the recombinant bacteriophage is an obligate lytic bacteriophage.

40. The recombinant bacteriophage of any one of claims 1-38, wherein the recombinant bacteriophage is a temperate bacteriophage that is rendered lytic.

41. The recombinant bacteriophage of claim 40, wherein the temperate bacteriophage is rendered lytic by the removal, replacement, or inactivation of a lysogeny gene.

42. A recombinant bacteriophage comprising at least 80% sequence identity to p6921.

43. A recombinant bacteriophage comprising at least 80% sequence identity to p6977.

44. A recombinant bacteriophage comprising at least 80% sequence identity to p6984.

45. A recombinant bacteriophage comprising at least 80% sequence identity to p00exe299.

46. A recombinant bacteriophage comprising at least 80% sequence identity to p004ke127.

47. A recombinant bacteriophage comprising at least 80% sequence identity to p00jce098.

48. A recombinant bacteriophage comprising at least 80% sequence identity to p00exe296.

49. A recombinant bacteriophage comprising at least 80% sequence identity to p00c0e103.

50. A composition comprising at least two of the recombinant bacteriophages of claims 1-49.

51. A composition comprising a first nucleic acid encoding a first Colicin, and a second nucleic acid encoding a second Colicin, wherein the first Colicin comprises Colicin Ib, and the second Colicin comprises Colicin 10, Colicin U, or Colicin K.

52. The composition of claim 51, wherein the second Colicin comprises Colicin 10.

53. The composition of claim 51, wherein the second Colicin comprises Colicin U.

54. The composition of claim 51, wherein the second Colicin comprises Colicin K.

55. A composition comprising a first nucleic acid encoding a first Colicin, and a second nucleic acid encoding a second Colicin, wherein the first Colicin comprises Colicin 10, and the second Colicin comprises Colicin U or Colicin K.

56. The composition of claim 55, wherein the second Colicin comprises Colicin U.

57. The composition of claim 55, wherein the second Colicin comprises Colicin K.

58. A composition comprising a first nucleic acid encoding a first Colicin, and a second nucleic acid encoding a second Colicin, wherein the first Colicin comprises Colicin U, and the second Colicin comprises Colicin K.

59. A composition comprising a first nucleic acid encoding Colicin Ib, a second nucleic acid encoding Colicin 10, and a third nucleic acid encoding Colicin U.

60. A composition comprising a first nucleic acid encoding Colicin Ib, a second nucleic acid encoding Colicin 10, and a third nucleic acid encoding Colicin K.

61. A composition comprising a first nucleic acid encoding Colicin Ib, a second nucleic acid encoding Colicin U, and a third nucleic acid encoding Colicin K.

62. A composition comprising a first nucleic acid encoding Colicin 10, a second nucleic acid encoding Colicin U, and third nucleic acid encoding Colicin K.

63. A composition comprising a first nucleic acid encoding Colicin Ib, a second nucleic acid encoding Colicin 10, a third nucleic acid encoding Colicin U, and a fourth nucleic acid encoding Colicin K.

64. A method of killing bacteria comprising introducing into the bacteria genetic material from the recombinant bacteriophage of any one of claims 1-49 or the composition of any one of claims 50-63.

65. A method of treating a disease in an individual in need thereof, the method comprising administering to the individual the recombinant bacteriophage of any one of claims 1-49 or the composition of any of claims 50-63.

66. The method of claim 65, wherein the disease is caused by a bacteria.

67. The method of claim 65 or 66, wherein the bacteria is Escherichia (e.g., E. coli).

68. A method of killing bacteria comprising contacting the bacteria with the composition of any one of claims 50-63, thereby killing the bacteria.

69. A method of killing a population of bacteria, the method comprising contacting the population of bacteria with one or more Colicins, wherein the one or more Colicins comprises Colicin Ib, Colicin 10, Colicin U, or Colicin K, or a combination of two or more thereof.

70. The method of claim 69, wherein the one or more Colicins comprises Colicin Ib.

71. The method of claim 69 or claim 70, wherein the one or more Colicins comprises Colicin 10.

72. The method of any one of claims 69-71, wherein the one or more Colicins comprises Colicin U.

73. The method of any one of claims 69-72, wherein the one or more Colicins comprises Colicin K.

74. A method of reducing resistance to a bacteriophage in a population of target bacteria, comprising administering the bacteriophage to the population of target bacteria, wherein the bacteriophage comprises comprising a nucleic acid sequence encoding a Colicin Ib, Colicin 10, Colicin U, or Colicin K;
wherein the population of target bacteria administered the bacteriophage comprising a nucleic acid sequence has a lower concentration of target bacteriophage at 24 hours following administration when compared to a second population of target bacteriophage administered a wildtype bacteriophage.

75. A method of treating a urinary tract infection in a subject in need thereof, the method comprising administering the recombinant bacteriophage of any one of claims 1-49 or the composition of any one of claims 50-63 to a subject in need thereof;
wherein the recurrent urinary tract infection comprises a Escherichia coli infection, wherein the first bacteriophage and the second bacteriophage target E. coli.

76. A method of reducing the levels of Escherichia coli in a subject, the method comprising administering the recombinant bacteriophage of any one of claims 1-49 or the composition of any one of claims 50-63 to a subject in need thereof, wherein the Escherichia coli concentration is or has been measured in a blood or urine sample of the patient.

77. Use of the recombinant bacteriophage of any one of claims 1-49 or the composition of any one of claims 50-63 for the treatment of a disease in a subject in need thereof.

78. Manufacture of a medicament the recombinant bacteriophage of any one of claims 1-49 or the composition of any one of claims 50-63 for use in treatment of a disease in a subject in need thereof.

79. A kit comprising the recombinant bacteriophage of any one of claims 1-49 or the composition of any one of claims 50-63.


SEQUENCE LISTING (184 sequences)

SEQ ID NO: 1 - DNA, 97 bp, unidentified
  misc_feature 1..97: note = Description of Unknown: Phage genome sequence
  source 1..97: mol_type = other DNA
acaagcggcacattgtgcctattgcgaattaggcacaatgtgcctaatctaacgtcatgccagccacaacggcgaggcgccaagaaggatagaagcc

SEQ ID NO: 2 - DNA, 35 bp, synthetic construct
  misc_feature 1..35: note = Description of Artificial Sequence: Synthetic oligonucleotide
  source 1..35: mol_type = other DNA
tttacagctagctcagtcctagggactgtgctagc

SEQ ID NO: 3 - DNA, 155 bp, Pseudomonas aeruginosa
  source 1..155: mol_type = other DNA
gatttttttcgggtgaggttgcgggctgttcggtaggtttataaacactgctatccaaagctatggacacgctcggctacgagaacagttggcgtgatggcctctagcaattagattgttatgcgacatccgcagacttggcagggagcgcacct

SEQ ID NO: 4 - DNA, 35 bp, synthetic construct
  misc_feature 1..35: note = Description of Artificial Sequence: Synthetic oligonucleotide
  source 1..35: mol_type = other DNA
tttacggctagctcagtcctaggtatagtgctagc

SEQ ID NO: 5 - DNA, 199 bp, Pseudomonas aeruginosa
  source 1..199: mol_type = other DNA
atccgagggatacgggccttgtcagcacggtgttgctaatgagagcctttgcccgggcaatagtacgggcagtgtgtagcggattgaaagacgctgaatcactgacaggcatgaagactatcgatagagtctgatagtgtcgccgccgcacagcggatagagtccacagtcattgaagtgttaatccgcgatcaagctc

SEQ ID NO: 6 - DNA, 216 bp, unidentified
  misc_feature 1..216: note = Description of Unknown: Phage genome sequence
  source 1..216: mol_type = other DNA
gacctagcttttatagcgggtttcgtggtttatagcccattgaaaaaaatctcacatctatatcacaggtgtgcactcgttcccgaaaggttctgagtctacttgatcaagtattgaaataccatcgtaaaggaaaaagacatgtctattcgtgatagcgaaaacaacaacggccaacagcagcagaccgcgcaaactgccgcccccgccccgcaa

SEQ ID NO: 7 - DNA, 182 bp, unidentified
  misc_feature 1..182: note = Description of Unknown: Phage genome sequence
  source 1..182: mol_type = other DNA
ttcaatttaagtagtaacgaggtcagcccggaatctttgggtattcttaaggtatttctgactcagtgtggttgggacagcttcactgtacattgcactggatttgttaatttcttataccggggcaccatgggcagcaaatcgtgttacgaattccgtctaaccaataagcgagctaaata

SEQ ID NO: 8 - DNA, 105 bp, synthetic construct
  misc_feature 1..105: note = Description of Artificial Sequence: Synthetic polynucleotide
  source 1..105: mol_type = other DNA
cgcggaacccctatttgtttatttttctaaatacattcaaatatgtatccgctcatgagacaataaccctgataaatgcttcaataatattgaaaaaggaagagt

SEQ ID NO: 9 - DNA, 161 bp, Pseudomonas aeruginosa
  source 1..161: mol_type = other DNA
cttcaagaattcgtattgaccccatagacagcttcgtcgacgcccgtcccggcccccttgggcttgccggacggcttatgtcatgatggcgccaccctcgcaggttcaaggccggctttcttcctctatgaacaaatcccttgcgctgactacgtaatcac

SEQ ID NO: 10 - DNA, 207 bp, Pseudomonas aeruginosa
  source 1..207: mol_type = other DNA
cttcaagaattcggggtattcctgatcctgcgccgctagcgccgcgcacggccactaggcccgcgccgatagccagtcgcgctcccggctggcacactactcccatttccgccggaaacgcgcgcaacgtaccggcaacgaacgtggaaagaccatgaaagactggctggatgagattcactggaacgccgtgacctacgtatgcac

SEQ ID NO: 11 - DNA, 59 bp, Escherichia coli
  source 1..59: mol_type = other DNA
gaaaattattttaaatttcctctagtcaggccggaataactccctataatgcgacacca

SEQ ID NO: 12 - DNA, 34 bp, synthetic construct
  misc_feature 1..34: note = Description of Artificial Sequence: Synthetic oligonucleotide
  source 1..34: mol_type = other DNA
atttatcacaaaaggattgttcgatgtccaacaa

SEQ ID NO: 13 - DNA, 32 bp, synthetic construct
  misc_feature 1..32: note = Description of Artificial Sequence: Synthetic oligonucleotide
  source 1..32: mol_type = other DNA
gtcgcgccccgcacgggcgcgtggattgaaac

SEQ ID NO: 14 - DNA, 32 bp, synthetic construct
  misc_feature 1..32: note = Description of Artificial Sequence: Synthetic oligonucleotide
  source 1..32: mol_type = other DNA
gtcgcgccccgcacgggcgcgtggagtgaaag

SEQ ID NO: 15 - DNA, 32 bp, synthetic construct
  misc_feature 1..32: note = Description of Artificial Sequence: Synthetic oligonucleotide
  source 1..32: mol_type = other DNA
gtcgcgccccgcacgggtgcgtggattgaaac

SEQ ID NO: 16 - DNA, 32 bp, synthetic construct
  misc_feature 1..32: note = Description of Artificial Sequence: Synthetic oligonucleotide
  source 1..32: mol_type = other DNA
gtcgcgccccgcatgggcgcgtggattgaaca

SEQ ID NO: 17 - DNA, 32 bp, synthetic construct
  misc_feature 1..32: note = Description of Artificial Sequence: Synthetic oligonucleotide
  source 1..32: mol_type = other DNA
gtcgcgccctacgcgggcgcgtggagtgaaag

SEQ ID NO: 18 - DNA, 36 bp, synthetic construct
  misc_feature 1..36: note = Description of Artificial Sequence: Synthetic oligonucleotide
  source 1..36: mol_type = other DNA
gtctaagaactttaaataatttctactgttgtagat

SEQ ID NO: 19 - DNA, 53 bp, synthetic construct
  misc_feature 1..53: note = Description of Artificial Sequence: Synthetic oligonucleotide
  source 1..53: mol_type = other DNA
tttacagctagctcagtcctagggactgtgctagcattaaagaggagaaaatg

SEQ ID NO: 20 - DNA, 34 bp, synthetic construct
  misc_feature 1..34: note = Description of Artificial Sequence: Synthetic oligonucleotide
  source 1..34: mol_type = other DNA
cggcagatggtcattgacctcggcataggtcaga

SEQ ID NO: 21 - DNA, 34 bp, synthetic construct
  misc_feature 1..34: note = Description of Artificial Sequence: Synthetic oligonucleotide
  source 1..34: mol_type = other DNA
ataatcgatcatttgacgactcctgtcttagcgt

SEQ ID NO: 22 - DNA, 34 bp, synthetic construct
  misc_feature 1..34: note = Description of Artificial Sequence: Synthetic oligonucleotide
  source 1..34: mol_type = other DNA
tgacatttcagcttttgtaagcgccataggttca

SEQ ID NO: 23 - DNA, 34 bp, synthetic construct
  misc_feature 1..34: note = Description of Artificial Sequence: Synthetic oligonucleotide
  source 1..34: mol_type = other DNA
gcctttgtgctgcagtttattgagctgccgttcc

SEQ ID NO: 24 - DNA, 34 bp, synthetic construct
  misc_feature 1..34: note = Description of Artificial Sequence: Synthetic oligonucleotide
  source 1..34: mol_type = other DNA
atgccggatgcggcgtgaacgccttatccggcct

SEQ ID NO: 25 - DNA, 34 bp, synthetic construct
  misc_feature 1..34: note = Description of Artificial Sequence: Synthetic oligonucleotide
  source 1..34: mol_type = other DNA
aaccgtctgtactgaagcgcaaccgttctcacgg

SEQ ID NO: 26 - DNA, 34 bp, synthetic construct
  misc_feature 1..34: note = Description of Artificial Sequence: Synthetic oligonucleotide
  source 1..34: mol_type = other DNA
gtaagattaaaaaactgatcgagctggttgaaga

SEQ ID NO: 27 - DNA, 34 bp, synthetic construct
  misc_feature 1..34: note = Description of Artificial Sequence: Synthetic oligonucleotide
  source 1..34: mol_type = other DNA
ttatgactcctccagtatcaaagtcctgaaaggg

SEQ ID NO: 28 - DNA, 34 bp, synthetic construct
  misc_feature 1..34: note = Description of Artificial Sequence: Synthetic oligonucleotide
  source 1..34: mol_type = other DNA
gtaaccgcggtctgcactctgcattcactgttcg

SEQ ID NO: 29 - DNA, 34 bp, synthetic construct
  misc_feature 1..34: note = Description of Artificial Sequence: Synthetic oligonucleotide
  source 1..34: mol_type = other DNA
cctatccttctggtcgactacacatgggccacgt

SEQ ID NO: 30 - DNA, 34 bp, synthetic construct
  misc_feature 1..34: note = Description of Artificial Sequence: Synthetic oligonucleotide
  source 1..34: mol_type = other DNA
tgatctgtctgcggacattaacgaacacctgatc

SEQ ID NO: 31 - DNA, 34 bp, synthetic construct
  misc_feature 1..34: note = Description of Artificial Sequence: Synthetic oligonucleotide
  source 1..34: mol_type = other DNA
tgtcatgcaagtccctcgggtcgagaagatcacc

SEQ ID NO: 32 - DNA, 34 bp, synthetic construct
  misc_feature 1..34: note = Description of Artificial Sequence: Synthetic oligonucleotide
  source 1..34: mol_type = other DNA
caatatcatggtcgtgtccaggcactggcagatg

SEQ ID NO: 33 - DNA, 34 bp, synthetic construct
  misc_feature 1..34: note = Description of Artificial Sequence: Synthetic oligonucleotide
  source 1..34: mol_type = other DNA
tatgaaaaaccgactaccgaacgtaagcgcgcta

SEQ ID NO: 34 - DNA, 34 bp, synthetic construct
  misc_feature 1..34: note = Description of Artificial Sequence: Synthetic oligonucleotide
  source 1..34: mol_type = other DNA
tgaacaacgaaatcatcctggtaacctgtggttc

SEQ ID NO: 35 - DNA, 25 bp, synthetic construct
  misc_feature 1..25: note = Description of Artificial Sequence: Synthetic oligonucleotide
  source 1..25: mol_type = other DNA
cattggtagccatgtttctttcctg

SEQ ID NO: 36 - DNA, 25 bp, synthetic construct
  misc_feature 1..25: note = Description of Artificial Sequence: Synthetic oligonucleotide
  source 1..25: mol_type = other DNA
atactggaggagtcataagaattcg

SEQ ID NO: 37 - DNA, 25 bp, synthetic construct
  misc_feature 1..25: note = Description of Artificial Sequence: Synthetic oligonucleotide
  source 1..25: mol_type = other DNA
cggcgaccagtgccgtagtattgat

SEQ ID NO: 38 - DNA, 98 bp, synthetic construct
  misc_feature 1..98: note = Description of Artificial Sequence: Synthetic oligonucleotide
  source 1..98: mol_type = other DNA
gtcgcgccccgcacgggcgcgtggattgaaacatttatcacaaaaggattgttcgatgtccaacaagtcgcgccccgcacgggcgcgtggattgaaac

SEQ ID NO: 39 - DNA, 230 bp, synthetic construct
  misc_feature 1..230: note = Description of Artificial Sequence: Synthetic polynucleotide
  source 1..230: mol_type = other DNA
gtcgcgccccgcacgggcgcgtggattgaaacatttatcacaaaaggattgttcgatgtccaacaagtcgcgccccgcacgggcgcgtggattgaaacaaccgtctgtactgaagcgcaaccgttctcacgggtcgcgccccgcacgggcgcgtggattgaaacatgccggatgcggcgtgaacgccttatccggcctgtcgcgccccgcacgggcgcgtggattgaaac

SEQ ID NO: 40 - DNA, 166 bp, synthetic construct
  misc_feature 1..166: note = Description of Artificial Sequence: Synthetic polynucleotide
  source 1..166: mol_type = other DNA
atttatcacaaaaggattgttcgatgtccaacaagtcgcgccccgcacgggcgcgtggattgaaacaaccgtctgtactgaagcgcaaccgttctcacgggtcgcgccccgcacgggcgcgtggattgaaacatgccggatgcggcgtgaacgccttatccggcct

SEQ ID NO: 41 - DNA, 219 bp, synthetic construct
  misc_feature 1..219: note = Description of Artificial Sequence: Synthetic polynucleotide
  source 1..219: mol_type = other DNA
gtctaagaactttaaataatttctactgttgtagatcattggtagccatgtttctttcctggtctaagaactttaaataatttctactgttgtagatatactggaggagtcataagaattcggtctaagaactttaaataatttctactgttgtagatcggcgaccagtgccgtagtattgatgtctaagaactttaaataatttctactgttgtagat

SEQ ID NO: 42 - DNA, 157 bp, synthetic construct
  misc_feature 1..157: note = Description of Artificial Sequence: Synthetic polynucleotide
  source 1..157: mol_type = other DNA
gaaaattattttaaatttcctctagtcaggccggaataactccctataatgcgacaccagtcgcgccccgcacgggcgcgtggattgaaacatttatcacaaaaggattgttcgatgtccaacaagtcgcgccccgcacgggcgcgtggattgaaac

SEQ ID NO: 43 - DNA, 289 bp, synthetic construct
  misc_feature 1..289: note = Description of Artificial Sequence: Synthetic polynucleotide
  source 1..289: mol_type = other DNA
gaaaattattttaaatttcctctagtcaggccggaataactccctataatgcgacaccagtcgcgccccgcacgggcgcgtggattgaaacatttatcacaaaaggattgttcgatgtccaacaagtcgcgccccgcacgggcgcgtggattgaaacaaccgtctgtactgaagcgcaaccgttctcacgggtcgcgccccgcacgggcgcgtggattgaaacatgccggatgcggcgtgaacgccttatccggcctgtcgcgccccgcacgggcgcgtggattgaaac

SEQ ID NO: 44 - DNA, 278 bp, synthetic construct
  misc_feature 1..278: note = Description of Artificial Sequence: Synthetic polynucleotide
  source 1..278: mol_type = other DNA
gaaaattattttaaatttcctctagtcaggccggaataactccctataatgcgacaccagtctaagaactttaaataatttctactgttgtagatcattggtagccatgtttctttcctggtctaagaactttaaataatttctactgttgtagatatactggaggagtcataagaattcggtctaagaactttaaataatttctactgttgtagatcggcgaccagtgccgtagtattgatgtctaagaactttaaataatttctactgttgtagat

SEQ ID NO: 45 - DNA, 289 bp, synthetic construct
  misc_feature 1..289: note = Description of Artificial Sequence: Synthetic polynucleotide
  source 1..289: mol_type = other DNA
gaaaattattttaaatttcctctagtcaggccggaataactccctataatgcgacaccagtcgcgccccgcacgggcgcgtggattgaaacatttatcacaaaaggattgttcgatgtccaacaagtcgcgccccgcacgggcgcgtggattgaaacaaccgtctgtactgaagcgcaaccgttctcacgggtcgcgccccgcacgggcgcgtggattgaaactgaacaacgaaatcatcctggtaacctgtggttcgtcgcgccccgcacgggcgcgtggattgaaac

SEQ ID NO: 46 - DNA, 3900 bp, synthetic construct
  misc_feature 1..3900: note = Description of Artificial Sequence: Synthetic polynucleotide
  source 1..3900: mol_type = other DNA
agtatctatcaggaattcgtaaacaaatattccctgtcgaaaacgctgcgctttgaattaattccacaaggcaaaacgctggaaaacatcaaagcgcgtggccttatcctggatgacgaaaagcgtgcaaaagactacaagaaagctaaacaaatcatcgacaaatatcatcagttttttattgaagaaattcttagctccgtgtgtatcagtgaagaccttcttcagaactatagcgatgtttacttcaagctgaagaaatcggacgatgataatctgcagaaagattttaagagtgccaaggatactatcaagaaacagattagtgagtacattaaggattccgagaagttcaaaaacctgttcaaccagaacctgatcgatgccaagaaagggcaggaatccgatctgatcctgtggcttaaacaatccaaagataacggcatcgaactgtttaaggcaaatagcgacatcacagatatcgatgaagcactggaaattatcaaatcgttcaaagggtggacaacctacttcaaaggatttcatgaaaatcgtaaaaacgtgtacagctcaaacgacatcccaacctctatcatttaccgcatcgttgacgataatttacccaaatttttggagaacaaagcgaaatatgaatctctgaaggataaggcgccggaagcgatcaattatgaacagatcaaaaaggacttggcggaggagttgacttttgatattgactataaaacgtcggaagtgaaccagcgcgtctttagcctggacgaagttttcgagattgcaaactttaataactacttaaatcaatccgggattactaagtttaatacgattattggcgggaaattcgttaacggtgaaaacaccaaacgtaaaggtatcaacgaatacattaatctgtatagccagcaaatcaacgataaaacactcaaaaaatataagatgtccgtgctgttcaaacagattttaagtgatacggaaagtaagtcatttgtgattgacaaactggaagatgactccgatgtagtgacgactatgcagtcattttatgaacagattgcggctttcaaaaccgtcgaagagaagtcgatcaaagagacgctcagcctcttatttgatgatctcaaagcccagaagctggatctttcgaaaatctactttaaaaacgataagtctttaaccgacctgtcccaacaggtgtttgatgactactccgtgattggtactgccgtgctggaatatattacccagcaaattgctccgaaaaacttagataacccaagtaagaaagaacaggaactgattgcgaaaaaaacggaaaaagcgaaatatctgagcctggaaaccattaaactggccctggaagaatttaacaaacatcgtgatatcgacaaacaatgccgtttcgaagaaatcctggccaattttgccgccattccgatgattttcgatgaaatcgcacagaataaggacaatctggcgcagatctcaattaaataccagaatcagggtaagaaagatctcctgcaggctagcgccgaagatgatgtgaaagcgatcaaagacctgctggaccaaactaataatctgctgcataaactgaagatctttcacattagccagtcagaagacaaggcgaacattctcgataaagatgaacacttctacctggttttcgaagaatgctacttcgaattagcaaacattgtccccctctataataaaattcgcaattatattactcagaaaccctatagcgatgaaaaattcaaactgaatttcgaaaactccaccctggcgaacggttgggataagaataaagagccagataataccgccattctgtttatcaaagacgataaatattatttaggcgtcatgaacaagaaaaacaacaaaatctttgacgacaaagccatcaaggaaaataagggtgaaggttacaagaaaatcgtttataaactgctgcctggggcgaacaaaatgctgccgaaagtgttttttagcgccaagagcattaaattttataatccgagtgaagacatcctgcgcatccgtaatcattcgacgcataccaaaaatggcagcccgcaaaaaggctatgaaaagtttgagttcaatattgaagattgtcgtaagtttatcgacttttacaaacaatcgatcagcaaacatcccgaatggaaagactttggcttccgtttctccgacacccaacgttataactcgatcgatgagttctatcgcgaggtcgaaaatcagggttataaactgacattcgaaaacatcagcgagtcatacattgactccgttgtaaatcagggcaaactgtacctcttccagatttataataaagatttcagcgcgtactcaaagggccgccccaatctccataccttatattggaaggcattgtttgatgagcgtaatctccaggatgtagtgtacaaactcaatggcgaggcggaactgttttatcgtaaacagagcattccaaagaaaattacccacccggcgaaggaagcgattgccaacaaaaacaaggacaatccgaaaaaggaaagcgtgttcgagtacgacctgattaaagacaaacgctttacggaagataagtttttctttcactgcccgatcacgattaactttaaatcgtccggcgcaaacaaatttaacgacgaaattaacttgctgctgaaagagaaagcgaacgatgtgcacattctgtcgatcgatcgtggtgaacgccatctggcgtattacacgctggtggatggcaagggtaacattatcaagcaggataccttcaatatcatcggtaatgatcgcatgaaaacgaattatcacgacaaactggctgctatcgagaaagatcgtgatagcgctcgcaaagactggaaaaaaatcaacaacatcaaggaaatgaaagaaggttatctttctcaggtagtacacgaaatcgccaaattagtcattgaatataatgctattgtagtgtttgaagatctgaactttggtttcaaacgcggtcgcttcaaggtcgaaaaacaggtttaccagaaattagagaaaatgttgattgagaaactgaactatctggtcttcaaggacaatgagtttgataagacaggtggcgttctgcgtgcgtatcagctgaccgccccgttcgaaaccttcaagaaaatgggtaaacagacgggtattatctattacgtgccggcaggcttcaccagcaagatttgcccggtcacgggcttcgtcaaccaactgtacccaaagtatgaaagtgtatccaagagccaggagttctttagcaagtttgataaaatctgctacaacctggataagggctatttcgagttcagcttcgactacaaaaacttcggcgacaaagccgcgaaaggcaagtggacgattgcctccttcggcagccgcctgattaactttcgcaactcggacaaaaatcataattgggatacccgcgaagtgtatcctacgaaagagctggagaaactgctcaaagactactcgatcgaatatggtcacggtgagtgtattaaagccgcaatttgcggtgaaagcgataaaaagtttttcgccaaactcaccagcgtattaaatactatccttcagatgcgcaattccaagacaggtaccgaactcgattatctgatctcgccggtggcggatgtgaacgggaacttctttgacagccgccaggcgccgaaaaatatgccacaagacgcagatgcaaatggagcttatcacattggtctgaaaggacttatgctcctgggccgtattaaaaataaccaggagggtaagaaacttaatctggttattaaaaacgaagaatattttgagttcgttcagaaccgtaacaactaa

SEQ ID NO: 47 - DNA, 2235 bp, Pseudomonas aeruginosa
  source 1..2235: mol_type = other DNA
atggacgcggaggctagcgatactcacttttttgctcactccaccttaaaggcagatcgcagcgattggcagcctctggtcgagcatctacaggctgttgcccgtttggcaggagagaaggctgccttcttcggcggcggtgaattagctgctcttgctggtctgttgcatgacttgggtaaatacactgacgagtttcagcggcgtattgcgggtgatgccatccgtgtcgatcactctactcgcggggccatactggcggtagaacgctatggcgcgctaggtcaattgctagcctacggcatcgctggccaccatgccgggttggccaatggccgcgaggctggtgagcgaactgccttggtcgaccgcctgaaaggggttgggctgccacggttattggaggggtggtgcgtggaaatcgtgctacccgagcgccttcaaccaccgccactaaaagcgcgcctggaaagaggtttctttcagttggcctttcttggccggatgctcttttcctgcttggttgatgcggattatctagataccgaagccttctaccaccgcgtcgaaggacggcgctcccttcgcgagcaagcgcggccgaccttggccgagttacgcgcagcccttgatcggcatctgactgagttcaagggagatacgccggtcaaccgcgttcgcggggagatattggccggcgtgcgcggcaaggcgagcgaacttcccgggctgttttctctcacagtgcccacaggaggcggcaagaccctggcctctctggctttcgccctggatcacgctctagctcatgggctgcgccgggtgatctacgtgattcccttcactagcatcgtcgagcagaacgctgcggtattccgtcgtgcactcggggccttaggcgaagaggcggtgctggagcatcacagcgccttcgttgatgaccgccggcagagcctggaggccaagaagaaactgaacctagcgatggagaactgggacgcgcctatcgtggtgaccactgcagtgcagttcttcgaaagcctgtttgccgaccgtccagcccagtgccgcaagctacacaacatcgccggcagcgtggtgattcttgacgaggcacagaccctaccgctcaagctgttgcggccctgcgttgccgcccttgatgaactggcgctcaactaccgttgtagcccagttctctgtactgccacgcagccagcgcttcaatcgccggatttcatcggtgggctgcaggacgtacgtgagctggcgcccgagccgcagcggctgttccgggagttggtgcgggtacgaatacggacattgggcccgctcgaagatgcggccttgactgagcagatcgccaggcgtgaacaagtgctgtgcatcgtcaacaatcgacgccaggcccgtgcgctctatgagtcgcttgccgagttgcccggtgcccgccatctcaccaccctgatgtgcgccaagcaccgtagcagcgtgctggccgaggtgcgccagatgctcaaaaagggggagccctgtcgcctggtggccacctcgctgatcgaggccggtgtggatgtggattttcccgtggtactgcgtgccgaggctggattggattccatcgcccaggccgcgggacgctgcaatcgcgaaggcaagcggccgctggccgaaagcgaggtgctggtgttcgccgcggccaattctgactgggcgccacccgaggaactcaagcagttcgcccaggccgcccgcgaagtgatgcgcctgcacccggatgattgcctgtccatggcggccatcgagcggtattttcgcatactgtactggcagaagggcgcggaggagttggatgcgggtaacctgctcggcctgattgagagaggccggctcgatggcctgccctacgagactttggccaccaagttccgcatgatcgacagccttcaactgccggtgatcatcccatttgatgacgaggccagagcagccctgcgcgagctggagttcgccgacggctgcgccgccatcgcccgtcgcctgcagccatatctggtgcagatgccacgcaagggttatcaggcattgcgggaagccggtgcgatccaggcggcggcaggtacgcgttatggtgagcagtttatggcgttggtcaaccctgatctgtatcaccaccaattcgggttgcactgggataatccggcctttgtcagcagcgagcggctatgttggtag

SEQ ID NO: 48 - DNA, 675 bp, Pseudomonas aeruginosa
  source 1..675: mol_type = other DNA
atggcctacggaattcgcttaatggtctggggcgagcgtgcctgcttcacccgcccggaaatgaaggtggaacgcgtctcttacgatgcgatcacgccgtccgccgcgcgcggcattctcgaggctatccactggaagccggcgattcgctgggtggtggatcgcattcaagtgcttaagccgatccgcttcgaatccatccggcgcaacgaggtcggcggcaagctgtccgctgtcagcgtcggtaaggcaatgaaggccgggcgtactaatggtctggtgaatctggtcgaggaggatcgccagcagcgcgcgactactctgctgcgcgatgtctcctatgtcatcgaggcgcatttcgagatgactgacagggctggcgccgacgatacggtgggcaagcatctggatatcttcaaccgtcgcgcacggaaggggcagtgcttccatacaccctgcctaggcgtgcgcgagtttccggccagttttcggttgctggaagagggcagtgccgagcctgaagtcgatgcctttctgcgcggcgagcgtgatctgggctggatgctgcatgacattgacttcgccgatggcatgaccccgcacttcttccgtgccctgatgcgcgatgggctgatcgaggtgccggccttcagggcggcagaggacaaggcatga

SEQ ID NO: 49 - DNA, 1764 bp, Pseudomonas aeruginosa
  source 1..1764: mol_type = other DNA
atgatcctttcggccctcaatgactattatcagcgactgctggagcggggtgaagcgaatatctcacccttcggctacagccaagaaaagatcagttacgccctgctgctgtccgcacaaggagagttgctggacgtgcaggacattcgcttgctctctggcaagaagcctcaacccaggcttatgagtgtgccgcagccggagaagcgcacctcgggcatcaagtccaacgtactgtgggacaagaccagctatgtgctgggtgttagtgccaagggcggagagcgtactcagcaggagcacgagtccttcaagacgctgcaccggcagatcttggttggggaaggcgaccccggtctgcaggccttgctccagttcctcgactgttggcagccggagcagttcaagcccccgctgttcagcgaagcaatgctcgacagcaacttagtgttccgcctagacggccaacaacgctatctgcacgagactccggcggccctggcgttgcgtacccggctgttggccgacggcgacagccgcgaggggctgtgcctagtctgcggccaacgtcagccgttggcgcgcctgcatccagcggtcaagggcgtcaatggtgcccagagttcgggggcttccatcgtctccttcaacctcgacgctttttcctcctacggcaagagccagggggaaaatgctccggtctccgaacaggccgcctttgcctacaccacggtgctcaaccatttgttgcgtcgcgacgagcacaaccgccagcgcctgcagattggcgacgcgagtgtggtgttctgggcgcaggcggatactcctgctcaggtggccgccgccgagtcgaccttctggaacctgctggagccacccgcagatgatggtcaggaagcggaaaagctgcgcggcgtgctggatgctgtggccacggggcggcccttgcatgagctcgactcgctaatggaggaaggtacccgcatttttgtgttagggctggcgcccaatacctcgcgactgtccattcggttctgggcagtcgatagccttgcggtattcacccagcatctggccgagcatttccgggatatgcaccttgagcctctgccctggaagacggagccggccatctggcgcttgctctatgctaccgcgcccagtcgtgacggcagagccaagaccgaagacgtactcccacaactggccggtgaaatgacccgcgccatcctgaccggcagccgctatccgcgcagtttgctagccaacctgatcatgcgcatgcgtgccgacggcgacgtctctggcatacgcgtcgcgctgtgcaaggccgtgctcgctcgcgaggcacgcctgagcggcaaaattcaccaagaggagctacctatgagtctcgacaaggacgccagcaaccccggctatcgcttggggaggctgttcgccgtgttggaaggcgcccagcgcgcagccctgggcgacagggtcaatgccactatccgtgaccgctactacggtgccgcgtccagcacgccagccacggttttcccgatactgctgcgcaacacacaaaaccacttggccaagctgcgcaaggagaagcccggactagcagtgaacctagagcgcgatataggcgaaatcattgacggtatgcagagccaattcccgcgttgcctgcgcctggaggaccagggacgctttgctattggttactaccaacaggcccaggcccgtttcaaccgtggccccgattccgtcgagtaa

SEQ ID NO: 50 - DNA, 870 bp, Pseudomonas aeruginosa
  source 1..870: mol_type = other DNA
atgaccgccatctccaaccgctacgagttcgtttacctctttgatgtcagcaatggcaatcccaatggcgacccggatgctggcaacatgccgcgtctcgatccggaaaccaaccaggggttggtcactgacgtttgcctcaagcgcaagatccgcaactacgtcagcctggagcaggaaagtgcccccggctatgccatctatatgcaggaaaaatccgtgctgaataaccagcacaaacaggcctacgaggcgctcggtatcgagtcagaggcaaagaaactgcccaaggacgaagccaaggcgcgcgaactgacctcttggatgtgcaagaacttcttcgatgtgcgtgctttcggggcggtgatgaccaccgagattaatgccggccaggtgcgtggaccgatccaactggcattcgccacgtctatcgacccggtattgcctatggaggtatccatcacccgcatggcggtgactaacgaaaaggatttggagaaggaacgcaccatgggacgcaagcacatcgtgccttacggcttgtaccgcgcccatggtttcatctctgccaagttggccgagcgaaccggcttttccgacgacgacttggaactgctatggcgcgctttggccaatatgttcgaacacgaccgctcggcggcacgtggcgagatggcagcgcgcaagttgatcgtcttcaagcatgagcatgccatgggcaatgcacccgcccatgtgctgttcggcagcgttaaggtcgagcgagtcgagggggacgcagttacaccagcacgcggtttccaggattaccgtgtcagcatcgatgcggaagctctgcctcagggcgtgagcgtgcgcgagtacctctag

SEQ ID NO: 51 - protein, 744 aa, Pseudomonas aeruginosa
MDAEASDTHFFAHSTLKADRSDWQPLVEHLQAVARLAGEKAAFFGGGELAALAGLLHDLGKYTDEFQRRIAGDAIRVDHSTRGAILAVERYGALGQLLAYGIAGHHAGLANGREAGERTALVDRLKGVGLPRLLEGWCVEIVLPERLQPPPLKARLERGFFQLAFLGRMLFSCLVDADYLDTEAFYHRVEGRRSLREQARPTLAELRAALDRHLTEFKGDTPVNRVRGEILAGVRGKASELPGLFSLTVPTGGGKTLASLAFALDHALAHGLRRVIYVIPFTSIVEQNAAVFRRALGALGEEAVLEHHSAFVDDRRQSLEAKKKLNLAMENWDAPIVVTTAVQFFESLFADRPAQCRKLHNIAGSVVILDEAQTLPLKLLRPCVAALDELALNYRCSPVLCTATQPALQSPDFIGGLQDVRELAPEPQRLFRELVRVRIRTLGPLEDAALTEQIARREQVLCIVNNRRQARALYESLAELPGARHLTTLMCAKHRSSVLAEVRQMLKKGEPCRLVATSLIEAGVDVDFPVVLRAEAGLDSIAQAAGRCNREGKRPLAESEVLVFAAANSDWAPPEELKQFAQAAREVMRLHPDDCLSMAAIERYFRILYWQKGAEELDAGNLLGLIERGRLDGLPYETLATKFRMIDSLQLPVIIPFDDEARAALRELEFADGCAAIARRLQPYLVQMPRKGYQALREAGAIQAAAGTRYGEQFMALVNPDLYHHQFGLHWDNPAFVSSERLCW

SEQ ID NO: 52 - protein, 224 aa, Pseudomonas aeruginosa
MAYGIRLMVWGERACFTRPEMKVERVSYDAITPSAARGILEAIHWKPAIRWVVDRIQVLKPIRFESIRRNEVGGKLSAVSVGKAMKAGRTNGLVNLVEEDRQQRATTLLRDVSYVIEAHFEMTDRAGADDTVGKHLDIFNRRARKGQCFHTPCLGVREFPASFRLLEEGSAEPEVDAFLRGERDLGWMLHDIDFADGMTPHFFRALMRDGLIEVPAFRAAEDKA

SEQ ID NO: 53 - protein, 587 aa, Pseudomonas aeruginosa
MILSALNDYYQRLLERGEANISPFGYSQEKISYALLLSAQGELLDVQDIRLLSGKKPQPRLMSVPQPEKRTSGIKSNVLWDKTSYVLGVSAKGGERTQQEHESFKTLHRQILVGEGDPGLQALLQFLDCWQPEQFKPPLFSEAMLDSNLVFRLDGQQRYLHETPAALALRTRLLADGDSREGLCLVCGQRQPLARLHPAVKGVNGAQSSGASIVSFNLDAFSSYGKSQGENAPVSEQAAFAYTTVLNHLLRRDEHNRQRLQIGDASVVFWAQADTPAQVAAAESTFWNLLEPPADDGQEAEKLRGVLDAVATGRPLHELDSLMEEGTRIFVLGLAPNTSRLSIRFWAVDSLAVFTQHLAEHFRDMHLEPLPWKTEPAIWRLLYATAPSRDGRAKTEDVLPQLAGEMTRAILTGSRYPRSLLANLIMRMRADGDVSGIRVALCKAVLAREARLSGKIHQEELPMSLDKDASNPGYRLGRLFAVLEGAQRAALGDRVNATIRDRYYGAASSTPATVFPILLRNTQNHLAKLRKEKPGLAVNLERDIGEIIDGMQSQFPRCLRLEDQGRFAIGYYQQAQARFNRGPDSVE

SEQ ID NO: 54 - protein, 289 aa, Pseudomonas aeruginosa
MTAISNRYEFVYLFDVSNGNPNGDPDAGNMPRLDPETNQGLVTDVCLKRKIRNYVSLEQESAPGYAIYMQEKSVLNNQHKQAYEALGIESEAKKLPKDEAKARELTSWMCKNFFDVRAFGAVMTTEINAGQVRGPIQLAFATSIDPVLPMEVSITRMAVTNEKDLEKERTMGRKHIVPYGLYRAHGFISAKLAERTGFSDDDLELLWRALANMFEHDRSAARGEMAARKLIVFKHEHAMGNAPAHVLFGSVKVERVEGDAVTPARGFQDYRVSIDAEALPQGVSVREYL

SEQ ID NO: 55 - DNA, 177 bp, synthetic construct
  misc_feature 1..177: note = Description of Artificial Sequence: Synthetic polynucleotide
  source 1..177: mol_type = other DNA
gaaaattattttaaatttcctctagtcaggccggaataactccctataatgcgacaccagtcgcgccccgcacgggcgcgtggattgaaacatttatcacaaaaggattgttcgatgtccaacaagtcgcgccccgcacgggcgcgtggattgaaacgcactcccgttctggataat

SEQ ID NO: 56 - DNA, 327 bp, synthetic construct
  misc_feature 1..327: note = Description of Artificial Sequence: Synthetic polynucleotide
  source 1..327: mol_type = other DNA
acccggggaaaattattttaaatttcctctagtcaggccggaataactccctataatgcgacaccagtctaagaactttaaataatttctactgttgtagatcattggtagccatgtttctttcctggtctaagaactttaaataatttctactgttgtagatatactggaggagtcataagaattcggtctaagaactttaaataatttctactgttgtagatcggcgaccagtgccgtagtattgatgtctaagaactttaaataatttctactgttgtagattcctcgctcactgactcgcttcgagctcggtacctgacagct

SEQ ID NO: 57-59 - skipped (3 entries)

SEQ ID NO: 60 - DNA, 804 bp, synthetic construct
  misc_feature 1..804: note = hDNaseI
  source 1..804: mol_type = other DNA
atgttaaagattgctgcttttaacattcaaacatttggtgaaacaaaaatgagtaatgctacattagtatcatatattgttcaaattttatctagatatgatattgctttagttcaagaagttagagatagtcatttaacagctgtaggtaaattattagataatttaaatcaagatgctcctgatacataccattacgtagtaagtgaacctttaggtagaaattcatataaagaaagatacttatttgtatatagacctgaccaagtaagtgctgtagattcatattattatgatgatggttgtgaaccttgtggtaatgatacatttaatagagaacctgctattgttagattcttttcaagatttacagaagtaagagaatttgctattgtacctttacatgctgctcctggtgatgctgtagctgaaattgatgctttatatgatgtatatttagatgtacaagaaaaatggggtttagaagatgtaatgttaatgggtgattttaatgctggttgtagttatgtaagaccttcacaatggtctagtattagattatggacaagtcctacatttcaatggttaattcctgattcagctgatacaacagctacacctacacattgtgcttatgatagaattgtagtagctggtatgttattaagaggtgctgtagtacctgattctgctttaccttttaattttcaagctgcttatggtttaagtgaccaattagctcaagctatttcagaccattatcctgtagaagtaatgttaaaacatcatcaccatcatcactaa

SEQ ID NO: 61 - DNA, 288 bp, synthetic construct
  misc_feature 1..288: note = Ipi
  source 1..288: mol_type = other DNA
atgaaaactttcaaagaatttacttctacgactaccccggtttctactattactgaagctactcttacttctgaagttattaaagcaaataaaggacgagaaggtaaaccgatgattagtctggttgatggtgaagaaatcaaaggtactgtttacctaggtgatgggtggtctgctaaaaaggatggtgctacaatcgttatctctcctgctgaagaaactgcgttgtttaaagctaaacacatttctgcggcacatctcaagattattgctaaaaatcttttgtaa

SEQ ID NO: 62 - protein, 267 aa, synthetic construct
  REGION 1..267: note = DNaseI
MLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKHHHHHH

SEQ ID NO: 63 - protein, 95 aa, synthetic construct
  REGION 1..95: note = Ipi
MKTFKEFTSTTTPVSTITEATLTSEVIKANKGREGKPMISLVDGEEIKGTVYLGDGWSAKKDGATIVISPAEETALFKAKHISAAHLKIIAKNLL

SEQ ID NO: 64 - DNA, 35 bp, synthetic construct
  misc_feature 1..35: note = BBa_J23102
  source 1..35: mol_type = other DNA
ttgacagctagctcagtcctaggtactgtgctagc

SEQ ID NO: 65 - DNA, 100 bp, synthetic construct
  misc_feature 1..100: note = Leader Sequence
  source 1..100: mol_type = other DNA
gataattagtgctgcgggtaggtaaggataaaaaagggtggcagcaggagattgagatggttttgctttattaacaacgggctaaacgtgtagtatttga

SEQ ID NO: 66-69 - skipped (4 entries)

SEQ ID NO: 70 - DNA, 139 bp, synthetic construct
  misc_feature 1..139: note = prom000011
  source 1..139: mol_type = other DNA
atttacgatattaatgtatcacgagctccgtcaatggttactattccagccgaagaactagatcgtcttcagaaaattgaagagcttctttgggaaattgaatctgatttgccatcaggattagaatcctggattgatt

SEQ ID NO: 71 - DNA, 250 bp, synthetic construct
  misc_feature 1..250: note = prom000012
  source 1..250: mol_type = other DNA
aaatatagcattgatgatgcttttaattatgaagaagaattcgaaacggaaattcaattcttaatgaaaaagtataatctcaagcgtcaggatattcgtatcctggccgaccacccatgcggtgaagatgttctttatattaaaggaaaatttgccggatatcttgatgaatatttttattctaaagatatgggcattgatatgcatatgagagttgtataaatagatatataattcagaggagacaatc

SEQ ID NO: 72 - DNA, 200 bp, synthetic construct
  misc_feature 1..200: note = prom000013
  source 1..200: mol_type = other DNA
aataacatagaaaagatttatcgtctttgtgataaaattgaaaaagaaaagaaatatctattttgtctatggcctattgttgacggaagagtaggcctagatgttcttgattatgaaacagaagacaaagtagatggcgcaacttttgataacgctttggatgttattgattggctcgaagaaaattatgtgaggtaaat

SEQ ID NO: 73 - DNA, 149 bp, synthetic construct
  misc_feature 1..149: note = prom000014
  source 1..149: mol_type = other DNA
aaaacatttaaaattgtcgtagaattttataacggcgaaggtgtagtccatcttaaagcggccaaccagtttgatgcggtaagagtatattgtcaatgctttgaaagttctaaacaagctataaaaattaaaagtgttgaggaaaccaa

SEQ ID NO: 74 - DNA, 248 bp, synthetic construct
  misc_feature 1..248: note = prom000015
  source 1..248: mol_type = other DNA
gatttatttgagatgttagaagataatcattctacgaataaccagaatgattctagtgattataagaaagagtaccgtatagtattacagaattatggaattgaagccccagatgctcttctagaagaactagcttcataccatcttgaccctccgccctgggctccctgggcaaaataattcaaaaagttgtttactttcctttctaacgatgatatgatagcttctgaagtatacgggaggctatc

SEQ ID NO: 75 - DNA, 300 bp, synthetic construct
  misc_feature 1..300: note = prom000016
  source 1..300: mol_type = other DNA
taatgatttagattctagcggaaatccttcacacactgccggtggaacagttggaacaacgtcagtaacgcttgaaaatgcaaatcttcctgcgactaaaactgacgaaagagttttaattgaagatgaaaatggatcagttattattggaggttgtcaatatgacccagatgaaactggtcctatatatacaaaatatcgtgaagactatgcaacaacaaactcttcacatactcctcctactaatattagtaatattcaaccgtctattactgtataccgttggataaggattgcata

SEQ ID NO: 76 - DNA, 300 bp, synthetic construct
  misc_feature 1..300: note = prom000017
  source 1..300: mol_type = other DNA
ggaatgcatgttggtggtgttcaggcacaacaaatgtcataccataaacatgctggtggttggggtgaatataacagaagtgaaggcccatttggcgcgtcagtttatcaaggatatcttggaactagaaaatattccgactgggataatgcttcatacttcactaatgatggatttgaattaggtggaccgagagatgctcatggtacacttaatcgtgaaggattaattggttatgaaactagaccatggaatatatcattaaactatattattaaagttcattactaaggattacaa

SEQ ID NO: 77 - DNA, 300 bp, synthetic construct
  misc_feature 1..300: note = prom000018
  source 1..300: mol_type = other DNA
aacttacttcacttataaatggaaataatcctgacggatcaactgttgaagaacgaggattaactaattctataaaaacgaatgaaaccaacattgcggcagtcacacatgaagtaaatacagctaaagacaatatatcctctttacagagcagcgttcaagctctacaagaagcaggttatattcctgaagctccaaaagatggccaagcttacgttcgtaaagacggcgaatgggtactactttctacctttttatcaccagcataacatggggccgcaaggccccaaaggattttaa

SEQ ID NO: 78 - DNA, 300 bp, synthetic construct
  misc_feature 1..300: note = prom000019
  source 1..300: mol_type = other DNA
atagttggtcattatctgaacgatacaataatccagaccataatttagtaggtcgtgttgtcggtcaagatccaaatgttaagcaaggtgcttataataatcgttgggtgaaagactatgcaacagctttagctaaagaattgaatggtcaaattttagcgcgtcaccagggaatgatgcttccaggcggtgttacgattgatggacaacgtttaatagaagaagctcgattagaaaaagaagcattacgcgaagaattatacttacttgaccctccatttggaatttttgtaggttaat

SEQ ID NO: 79 - DNA, 300 bp, synthetic construct
  misc_feature 1..300: note = prom000020
  source 1..300: mol_type = other DNA
aacaaatgctaatggacgcagccaagatttttcttgagacggcaaagaatgctgattctcctcgtcacatggaagtatttgcaactcttatggggcaaatgactacgacgaacagagaaatactgaagcttcacaaagacatgaaagatattacatctgaacaggttggcaccaaaggcgctgttcctacaggtcaaatgaatattcagaatgcgacagtattcatgggttcaccaacagaattaatggacgaaattggtgatgcttacgaggctcaagaagctcgtgagaaggtgataa

SEQ ID NO: 80 - DNA, 300 bp, synthetic construct
  misc_feature 1..300: note = prom000021
  source 1..300: mol_type = other DNA
agatgcagttaaaaataagacgttatttgagaaaatgacatcgagtttaactaacgttcttgtagtttcaaacccgacaatttggatggtgaaaaactttggtgcaacatctaagtttgatggaaaaacggaaatattcggtccatgtcaaatccagagtatcagatttgataaaacacctaatggtaactttaacggattagctattgctccaaatctccctagtacatttactctcgagattactatgagagaaattatcacgttaaaccgtgcttctttatatgcggggacttttta

SEQ ID NO: 81 - DNA, 300 bp, synthetic construct
  misc_feature 1..300: note = prom000022
  source 1..300: mol_type = other DNA
taaggtgacttatacttgtaatctatctaaacgggggacctctctagtagacaatcccgtgctaaattgtaggactaccctttaataatttcatcaggattagttacttaccgtgtaaaatctgataaatggaattggttctacataaatgcctaacgactatccctttggggagtagggtcaagtgactcgaaacgatagacaacttgctttaacaagttgaagatatagtctgatctgcatggtgacatgcagctggatataattccggggtaagattaacgaccttatctgaacata

SEQ ID NO: 82 - DNA, 300 bp, synthetic construct
  misc_feature 1..300: note = prom000023
  source 1..300: mol_type = other DNA
gtttggtaacacacttgattcgctttaccaagattggattacttatccaacgaccccagaagcacgtaccactcgctggacacgtacatggcagaaaaccaaaaactcttggtcaagttttgttcaggtatttgacggaggtaaccctcctcaaccttcagatataggagcgatcccatctgataatggaataataggtaatcttactattcgtgatttcttacgaattggtaatgttcgcattattcctgacccagtgaataaaactgttaaatttgaatgggttgaataagaggtatt

SEQ ID NO: 83 - DNA, 300 bp, synthetic construct
  misc_feature 1..300: note = prom000024
  source 1..300: mol_type = other DNA
ttatccatttaaaggaaaaattgactataaagattttacaaatctatcagtacgtgttatagtaactgaagtagacaaaaatctaacgaagttcgaatctgaactagaaaaagttgtgcattcattacgagttgtgtcaaagattgataactctgtcgagtcagatgacagtgaagaagttgaagttcaatctcttcagacgttgatggaagaatacattaatgcaattccagacatcactgattctgaccgtgaagcacttattcaatatgcaaatcagttatatgtagaggcaacaca

SEQ ID NO: 84 - DNA, 300 bp, synthetic construct
  misc_feature 1..300: note = prom000025
  source 1..300: mol_type = other DNA
agaatatgctaatgcagtttctgactatgagtattctgctcgggaaagaggtacagctttcgcaaaggaagaaatgaaaatcatggttgatgctcacacaaagcttcagaattttattgaaaacgtcatttaatggtttacaagttggcaagattatgatatagtaatcttgccaactgccaaggagaagagaatgaaagttttgtttgttgtgtatgtgatgattcaatataattatccaatgtttacttataatctggtgaacaacattattgatattattcaaaggagtatgtaatt

SEQ ID NO: 85 - DNA, 287 bp, synthetic construct
  misc_feature 1..287: note = prom000026
  source 1..287: mol_type = other DNA
gccttaaaagcaacggcactatttgccatgctaggattagcgtttgctttatctccaccaattgaagcgaatgtcgatcctcattttgataaatttatggaatctggtattagacacgtttatatgctttttgaaaataaaagcgtagaatcatctgaacagttctatagttttatgcgaacgacttataaaaatgacccgtgctcttccgattttgaatgtatagagcgaggcgcggagatggcacaatcatacgctagaattatgaacattaaattggagactga

SEQ ID NO: 86 - DNA, 300 bp, synthetic construct
  misc_feature 1..300: note = prom000027
  source 1..300: mol_type = other DNA
cggtctgttatttaaacgacgattatgattatcttggcgtttatagtttaagtgatgcacggtttaaacgtaatttacaaaagtcaaatttattttatattgatactacggtaaaatttcagggcaagaaatatttctttactcttatagttgattctgaaacgaagcatgagaataaacgtattcttagtaaaaagaatatcttaactattgttgatgatctttttgataaattcgtagaaaatcccaattttgaaagcgatttattactagaaaaatttgttaaggaatgtagagaat

SEQ ID NO: 87 - DNA, 300 bp, synthetic construct
  misc_feature 1..300: note = prom000028
  source 1..300: mol_type = other DNA
tgaaatcgccaagaaagtagttgagttagatgatgctcgtcaagaacttgcggttaaattggaatatatccgtgaaactcgtgcagcaaatgcccttggaattagtactgccgatgatgtagttgaaattgcagcactgactaaggttgatattgaagatacccttgctcgagttgaaacctttaacggtaatatttctggggttgaaactacctctgccgatgttcaggaatacattaattctctgaaataatgataaggggcttcggccccttatacttggagtaaataggaatgaaa

SEQ ID NO: 88 - DNA, 300 bp, synthetic construct
  misc_feature 1..300: note = prom000029
  source 1..300: mol_type = other DNA
aaaatcgtattaacactgcatctaagctaaaagctgctgcagaagctataataagtaaactaggtaaataattttaaatccctatctaaatgatagggctttttggtatctaggcctttctggacctctctaggcatcatttagtttataccctttataatatattatcctatcctttaattgccaatccctgccctagaattccctaaaaattttttcacaaaactgtttacatctctgttcttccatggtactatacaactatcaactactgatacagaaaacaacttggagaatgaa

SEQ ID NO: 89 - DNA, 297 bp, synthetic construct
  misc_feature 1..297: note = prom000030
  source 1..297: mol_type = other DNA
actgctataactccgcaagaatacatggcgtctcttaaagaaaaatataatctttctgcaacagaaacacttttcgatttaccagaaaaccttcaactaaaatttcaggtagaatttcaaaaattagttcacccagaacaaaaacactttactgcagtcgttaagtcaattaatgcagatggattgacaattttcacccgacaaatagtactaatttaagcaaggggcttcggccccttaattggagtataatatatcaagagcctaataactcgggctataaactaaggaatatct

SEQ ID NO: 90 - DNA, 300 bp, synthetic construct
  misc_feature 1..300: note = prom000031
  source 1..300: mol_type = other DNA
ttgagcgaaagtactaaagatctgactgagtctcaaaaagaaaaagtctctgctctggtcgaaggtatggattattcagatgcattctcaagtaaattgagtgcaatcgtagaaatggtgaagaaatctaataaagatgaaagcactattactgagagtataaatactcctgatactgaagcagccggactgaatttcgtcactgaagctgtagaagataaatctgcacaaggtgcagaagatattgtaagtgtatatgcgaaagtcgcatctcgtttctaattttaaaggttaacacaa

SEQ ID NO: 91 - DNA, 325 bp, synthetic construct
  misc_feature 1..325: note = prom000032
  source 1..325: mol_type = other DNA
ttacgtagatgttttaatgaatcatggatggaaacttcgcggtcatccaactaaaaattgtcatatgttcactgatggagatattgaagagcttcatgaaatggcagaagcaataggaatgaaacgttcttggtttcaagataaacgcattaaacattatgacttacacgctcgccgacgccaaaaagctgtagaacttggagctgtagaagtatctcgccgtgaagcagtaaaaatttggcgaacgttaaaataaattgtttacagaagggtagtagtgtgatactattaccctatcaaaacaaatgtgagattggagaataaa

SEQ ID NO: 92 - DNA, 300 bp, synthetic construct
  misc_feature 1..300: note = prom000033
  source 1..300: mol_type = other DNA
tgtattagtaggtcctgtgacagctgtatcatttataatcctaatgattattggaatagttatagatgttactactgatattgaatcagacgcagtatttctgttagtattaattcttccattagtagttccatttttattagtacctgtaaattgggtaggatactggtatcaaggaagacattatcgtaaacgcgtatgcgaatggaaagctcagtgtaaaaagattaaaaaggaacatcagcttaaacttgctgcgtatgaatttaatgaaattatgaaatttgttaaggaatcacg

SEQ ID NO: 93 - DNA, 300 bp, synthetic construct
  misc_feature 1..300: note = prom000034
  source 1..300: mol_type = other DNA
ctattgtagtgtcaaggtcaggtgcttattctgaaatgacttataggaatggctatgaagaagctattcgtcttcaaactatggcgcaatatgatggctatgctaaatgttctactgtcggtaattttaacttgactcctggtgttaaaattatttttaatgatagtaaaaaccaatttaaaacagaattttacgttgatgaagttatccatgaattatcaaataataattcagtaactcatctatatatgttcactaatgcaacgaaactggaaacaatagacccagttaaggttaaaa

SEQ ID NO: 94 - DNA, 300 bp, synthetic construct
  misc_feature 1..300: note = prom000035
  source 1..300: mol_type = other DNA
gctaaaaatgctctgactgctaacaaactggttgtagatggtattgaatatgatatctgtggagttcgtgaagaaaaacctggtgttctgactttcttcacaatgatttttaaatttaaaggtgacacagaattcaaacagtttgattttgcccatgaagatgaaatcgaagttcgtaatctgaacattaagtaagtactttattagagctcttgaaaaagagtgcaaaaaagtgtttacttctgctttaaacatgatactatagacctatcaaataaatgaactgaaacggagattaaa

SEQ ID NO: 95 - DNA, 300 bp, synthetic construct
  misc_feature 1..300: note = prom000036
  source 1..300: mol_type = other DNA
atgctcgtgaatttcttgacgaagaaaccggcgagatgattcgcgaagaaaaatcttggcgtgcaaaagatactaactgcactacattctggggtcctttatttaagcatcaaccattccgagatgctattaaacgtgcttatcagttaggtgctattgatagtaatgaaattgttgaagctgaagttgatgaattgattaactcaaaggttgaaaaatttaaatctccagaaagtaaaagtaaatcagctgctgatttagaaactgacctcgaacagttaagtgatatggaagaattta

SEQ ID NO: 96 - DNA, 300 bp, synthetic construct
  misc_feature 1..300: note = prom000037
  source 1..300: mol_type = other DNA
taagtgatgaagctcattttaattatctgatggctgctgttcctcggggtaaaagatatggtaaatgggcaaaactggttgaagattccaccgaagtattgattattaagttacttgctaagcggtatcaagttaatacaaatgatgcaattaactataaatcaattcttactaaaaatggaaaactatctttagtattaaaagaactaaaaggtttagtcacgtatgattttttgaaagaagtgactaagaacgtaaaagaacagaaacaactcaaaaaactagcattggaatggtaaa

SEQ ID NO: 97 - DNA, 300 bp, synthetic construct
  misc_feature 1..300: note = prom000038
  source 1..300: mol_type = other DNA
atcgtaattaacggttttaataaagtagaagattctgctctgacccgtgttaaatattctttgactcttggtgattatgatggtgaaaatacatttaatttcattatcaatatggcaaatatgaaaatgcaaccaggaaattataaacttctgctctgggcaaaaggtaaacaaggtgctgctaaatttgaaggtgaacacgcgaattatgtggtagctcttgaagctgattctacccacgatttttaataaagggcttcggccctttataatttacactaaaacttgaatgaggaaatt

SEQ ID NO: 98 - DNA, 300 bp, synthetic construct
  misc_feature 1..300: note = prom000039
  source 1..300: mol_type = other DNA
tttcagttaaaaatctgcatcacgttgttttagcacacggtgttaaatctaaaattattgtattgcaaacaataggtcgcgtattacgtaaacatggttctaaaacaatcgcaacagtctgggacctcatagatgactgtggtgtcaagccaaaatctgctaatactaaaaagaaatatgttcacttgaactatcttttaaaacacggcattgatcgtattcagcgctacgcagatgaaaaatttaattacgtaatgaaaacagtcaatttataagggcttcggccctttggagaaaaag

SEQ ID NO: 99 - DNA, 300 bp, synthetic construct
  misc_feature 1..300: note = prom000040
  source 1..300: mol_type = other DNA
gccgctaagaaagctgataaagttgctgatgatttggatgcattcaatgttgatgacttcaatacaaaaactgaagatgattttatgagctcaagctctggcagttcatctagtgctgatgacacagacctggatgaccttttgaatgacctttaatagattatattactaattaattggggaccctagaggtccccttttttatttcaaaaatttttcataaaacggtttacatccttgtccttccatggtactatacaactatcggcaatactgctgataattaaagaggaaaataat

SEQ ID NO: 100 - DNA, 300 bp, synthetic construct
  misc_feature 1..300: note = prom000041
  source 1..300: mol_type = other DNA
tataataaattttggtcagaagacgaagaaaaagaccttttaaatcttttagactctttaaatgacagaggaataaaatttggactgtcgaatgttttagagcaccacggaaaggaaaatactcttcttaaagaatggtctaaaaaatacactgttaagcatcttaataaaaaatacgtctttaacatatatcattccaaagaaaagaatggaactgatgaagtatatatttttaattaattgcttatatattcaaatggtataattatttaacttattaatgaattgaaaggaaaaata

SEQ ID NO: 101 - DNA, 300 bp, synthetic construct
  misc_feature 1..300: note = prom000042
  source 1..300: mol_type = other DNA
caaagcatcacgcttatggaatgttccaaaattatttgcctactatgcgagcgagagtcaaggaacttggttataatatgaccgatgctgaaataaaaagaatgttgaataaacggtccaattcagcttcctgggcgtacattgaactttcttattggttaaatatacataagggcgatataagaaaagcaatatcctcttataattcgggatggaatgttaaagctggttctaaatatgcttctgaagtcctagaaaaggctaattaccttaaaaataataaacttttggaaatagtaa

SEQ ID NO: 102 - DNA, 300 bp, synthetic construct
  misc_feature 1..300: note = prom000043
  source 1..300: mol_type = other DNA
aatcaaagaaactgaatcagcatttcgtttagcttttgccaaggcacatttcattaagaaagtaatttcaggtgaaattgttgtacaaggtaaaactcgcaaagaactgaccgaagaactttctaaaattgatatgtattcttcttatgttgataaactagttggaatgaacatttttcatatgacttccgacgaagcaaagaaacttgctgaagaagctaaagccaaaaaagaagaaaacgaatattggaaaactactgatgtagttacagaatacaccaaagatttagaggaaatcaa

SEQ ID NO: 103 - DNA, 239 bp, synthetic construct
  misc_feature 1..239: note = prom000044
  source 1..239: mol_type = other DNA
attgaattgagttggtaccagtttaaatctcttatgacaaatgttaaaactgtcattgaagaaaatcagggtcctgaaaatattactattcgcgaaaaagctttaaagatagtatacagtcttgaagaaatacaaaaagatattgaatctatggcaaaatttattgatgaacctattaataaagtttatattcaagattatactgtaggtcaaattcgcgatttagcaaggaaaattta

SEQ ID NO: 104 - DNA, 235 bp, synthetic construct
  misc_feature 1..235: note = prom000045
  source 1..235: mol_type = other DNA
gaaataactaaagatcagttttatcttcttcaagataaagtaagcgaaatttatgaaattgcttatagcaaaaatcgtgaaactgtaaaaatagaatctagtaagttgatgcttcaattagaagaaattgaacgagatttaattgcgttagaattcttttgtggtgaagtgaaaactgttacaatcaatgattatattttaggcgaaattagctatctttataaggcgattatta

SEQ ID NO: 105 - DNA, 300 bp, synthetic construct
  misc_feature 1..300: note = prom000046
  source 1..300: mol_type = other DNA
tcggagggggaagaagaaagaaaagaacgtctttttaatgaatctcttaagataattaaatctgccatggaaaatgttatccaggagattgtcattaaactagaagatggttctacacacattgtgtatgtgacaaaattagattgggttgatggaaaagtcgtaatggactttgctgttcttgaccaagaaagaaaagctgagttagctcctcatgtagaaaaatgtattacaatgcaactacaagatgcatttaataaaaggtcaaagaaaaagtttaaattcttttaaggagtaagt

SEQ ID NO: 106 - DNA, 195 bp, synthetic construct
  misc_feature 1..195: note = prom000047
  source 1..195: mol_type = other DNA
gaatattcaactggacagcatttattaacttttcctgaaataaaacgatatattctgagaaataatttttctaatgaagagcatatagttactgaatctatgcttaggaatgcatttaaagcagaatatacaaaaataatgtccaatagaaatgaagcttggactgttactgattattatgactaaaggtgtatt

SEQ ID NO: 107 - DNA, 300 bp, synthetic construct
  misc_feature 1..300: note = prom000048
  source 1..300: mol_type = other DNA
gcaacaacgatgcgatggacaaatgactgggagtattcaaaaaatcataagaagcccatggtgacaagaaaggctcatatgttagtgttaatagaccgtgagcagattaaagcccgagaagccctccagaatcataaaaaggctgcctttgaatggtttatggataacactgctcctgagactaagaaagcagtgagcgcatggttcagtggaaaaaattgtgaaagaagtttcttttagtggtttacaagactgttcctctgtggtactatacaactatcaactacggaggaacacaaa

SEQ ID NO: 108 - DNA, 300 bp, synthetic construct
  misc_feature 1..300: note = prom000049
  source 1..300: mol_type = other DNA
gatgttattctttcaactgacatgatgcaaaagcgcggtctttctgatgaagaaacttataaagcattatcatggatgaaagtcaatgttaaccgtttacgcaatgtttcactgcgtactgcactttatcttgctgactttattatgaccgacaaaaacggttggcaagaaatcgctgaggttactcttctgaaataaattcataagaggacttctatgacaaaaaggcagttcagaaatagattatatggactgccatcaaaaagatgactagaattaaactggtaaatggaggtaatg

SEQ ID NO: 109 - DNA, 300 bp, synthetic construct
  misc_feature 1..300: note = prom000050
  source 1..300: mol_type = other DNA
atttacgataaagttaagtctattaaagaccgtagtgttattaaacgtgaagttggtattattgctcaggaccttgaaaaggaattaccggaagctgtatctaaagttgaagttgatggatctgatgttctgacaatttctaactctgctgtgaatgctcttttaattaaggctattcaggaaatgagtgaagaaattaaagaattgaaaactcctttctttactaaaattgctcgcaaaattagtaaatattttaaattctaacaacaagggggaaatgccccctttggagtataaatt

SEQ ID NO: 110 - DNA, 300 bp, synthetic construct
  misc_feature 1..300: note = prom000051
  source 1..300: mol_type = other DNA
gaactgtataagtttaacctatttttaggtaaaacggcagaaacttataaaaattggaacaaaggcggaaaagctccatggagtgatttttgggatgctaaatcacagtttagtaaagtgaaagcacttcctgcatcaacattccataaagcacagggcatgtctgtagaccgtgctttcatttatacgccttgtattcattatgcagatgctgaattggctcaacaacttctttatgttggtgttacccgtggtcgttatgatgtgttttatgtatgattaaatttgaggaagctattc

SEQ ID NO: 111 - DNA, 244 bp, synthetic construct
  misc_feature 1..244: note = prom000052
  source 1..244: mol_type = other DNA
aaaattgttatggggtgcttagcagtttgtttagttgcattagcagcagttccatttgttagtgttgaaaatgatactcaacctgtgattgaatctagcaccgttattcacactaatggtaaaatatcagttaaaattgatgataatcttcatgtgaatactaatggaacgctgggtgttcaattaggtaatatctgtgtaagcactactggagtaattactacttgtatttgaggaaattatt

SEQ ID NO: 112 - DNA, 300 bp, synthetic construct
  misc_feature 1..300: note = prom000053
  source 1..300: mol_type = other DNA
atgctggtattttatcactggttactaacgatcgtggtgctattgatgatgttcttgagtctctcaaaaataaagatgttaaacaactcagagctttagcaccaaaatatgcagctgattattcgtggttcgtaggtaaacttgccgaagaaatctattcacgtgtaactccgcagagtattattcgtatgtacgaaattgtcggcgaaaataatcagtatcatggtattgcagctaatactgaattgcatttagcttatcttttcattcagttagcatgtgaaatgcagtggaagtgat

SEQ ID NO: 113 - DNA, 300 bp, synthetic construct
  misc_feature 1..300: note = prom000054
  source 1..300: mol_type = other DNA
taaagaaatagatggttatacttatgatattaatgacgtttatgtatgtcaaagattggaatttcaataccaaggaaatacattttattttagacctcctggaaaatttgaacaatttttaactgtaagcgatatgttatccaaatgcttgcttaaggtcaatgatgaagttaaagaaattaattttcttgagatgccagcatttgttttaaaatgggcaaatgatatttctacaactttagcaattcctggccctaatggtccaataaccggaattggcaatattattggattatttga

SEQ ID NO: 114 - DNA, 300 bp, synthetic construct
  misc_feature 1..300: note = prom000055
  source 1..300: mol_type = other DNA
accagcagttaaaaaagaacttgcttctagatttgctaaaattgatgccacttatcaagagcttaagaaaaatcaacctgaggccaaacctgaaacttctgctaaatcaccagaagcaaaacaggtccaggtgattgaaaagaacaaagcacaacaagctcctgttcaacaagcatctccttcaatcaataatactaataatgttattaagaaaaatactgtcgttcataatatgacacctgtcacaagcacaactgctcctggtgtatttggtgcgacgggagttaattaaggaataat

SEQ ID NO: 115 - DNA, 300 bp, synthetic construct
  misc_feature 1..300: note = prom000056
  source 1..300: mol_type = other DNA
agtgaagaagagttttattgtttccgcgaatataaagaaccgacttctgaagaggatgaagtcaaagacaaggtttctggcgtaacaaaaattcactgcattgttgacgaaaacaatgcagatgaaatcattgaacttttgcgaaaaactttcaaaaagtagtttacagcagggtagtagtgtgatactattaccctatcaaaactaatggagaaaagaaaatgttagcaccttatattatggcagcagttatgttggtttgtttatatcttttgattaaagcttgctaaggagaataaa

SEQ ID NO: 116 - DNA, 300 bp, synthetic construct
  misc_feature 1..300: note = prom000057
  source 1..300: mol_type = other DNA
tcatattgtagctaagatgtgtaatcttattccgggagatttgatattttccggtggtaatactcatatctatatgaatcacgtagaacaatgtaaagaaattttgcgtcgtgaacctaaagagctttgtgaactggtaataggcggattgccttataaattccgctatctttctactaaagaacaattggaatacattcttaaactcaggcctaaagatttcgttctcaaagattatcagtcccacggcgtcttgaaaggaaaaatggcggtgtaattttaatttaattgtgaggattt

SEQ ID NO: 117 - DNA, 300 bp, synthetic construct
  misc_feature 1..300: note = prom000058
  source 1..300: mol_type = other DNA
gcttcctgatgcggttgaggagatgaaagtctttttagaaaatcagcttgcgaaatatgattgcgatgtgttcattaatcagactcaacctaatgttcatattaacaactgtaaatgctatatcatcgttaatcctttaacgggaaaacatcgtcttggaattagtaatccaaatcgtagcgcatcggatatggcagaagatgttgaggcatgctttaaaatttctaaatctccggctgaacatcatattttaattaacggtctttctcaagacgatattatagaggttattaaaacttt

SEQ ID NO: 118 - DNA, 300 bp, synthetic construct
  misc_feature 1..300: note = prom000059
  source 1..300: mol_type = other DNA
aatgacttcaattgaatttactagtgccgttagaatagctctacaggaaatggttgtaaaatttattgcaattgattcatttgaagaccatcctaccataggaaataaaatacaagttaagtatttagataaccaagaacatatcttagaacaatactctgataaaggaattactttcaaacaggaaataatttctccttctaaacctgggtatggaacttggcaattattaggtgcgcaaactgttacgctagatagtcacacacaacctacagtattttattattttgagagagtagc

SEQ ID NO: 119 - DNA, 300 bp, synthetic construct
  misc_feature 1..300: note = prom000060
  source 1..300: mol_type = other DNA
ttgctaataataaagataaagttctgtatcagtcctgtcatattcttcagaaaaaaggactatactatatcgttcattttaaagaaatgcttcgtatggatggtcgccaagttgaaatgacagaagaagatgaagttcgtcgtgattcgattgcatggctgttagaagattggggactgattgaaatcgttcctggtcaaagaacttttatgaaagatttaactaataatttccgagttatttcttttaaacaaaaacatgaatggaaactcgttcctaaatatacgattggtaattaat

SEQ ID NO: 120 - DNA, 300 bp, synthetic construct
  misc_feature 1..300: note = prom000061
  source 1..300: mol_type = other DNA
ctcggctttctaaatcaaaacaaacctctgacgttaagctaacgattgtagcactcaaagctcgtattgatggttctcgtatagcagaaggcgctgaagttgttaaattgaacgttcttcttaaaggctctgattggaaaactgtgaaaaagttgtcagaagcagaaatgcaatatgatatgtgtgataaaattattcaaggtgtagagcggtatcaaaacttgtcttttattgataaacttaaactgaaaagaggatacccgttaaattgttcaatttttaaacttatccgaggataat

SEQ ID NO: 121 - DNA, 300 bp, synthetic construct
  misc_feature 1..300: note = prom000062
  source 1..300: mol_type = other DNA
ctattaagcatattcaagacatgcgagcatttgaggctggaaaataatgagatatagcattgatgatgcttttaattatgaagaagaatttgaaacggaaattcaattcttaatgaaaaagcataatcttaagcgtcaggatattcgtatcctggccgaccacccgtgcggtgaagatgttctttatattaaaggaaaatttgccggatatcttgatgaatattttttattctaaagatatgggcattgatatgcatatgagagttgtataaatagatatataattcagaggagacaatc

SEQ ID NO: 122 - DNA, 300 bp, synthetic construct; identical to SEQ ID NO: 90
  misc_feature 1..300: note = prom000063
  source 1..300: mol_type = other DNA

SEQ ID NO: 123 - DNA, 300 bp, synthetic construct
  misc_feature 1..300: note = prom000064
  source 1..300: mol_type = other DNA
tgaaatccaaaaaatgaccgaagaaggtaaagattggaaaactgaagatccagacagtaaatactacctgcatcgttacactcttcagaagatgatgaaagactatccagaagttgatgttcaagaatcacgcaatggatacatcattcataaaactgctttagaaactggtatcatctatacctatccataatcataatcataaggggcttcggcccctttcttcattttgaaagcacacaaaacatactcagaaaatgatgtatataatggcatcaactcgataacatgagattgatt

SEQ ID NO: 124 - DNA, 300 bp, synthetic construct
  misc_feature 1..300: note = prom000065
  source 1..300: mol_type = other DNA
gattgttaataacttcgaccgcatgtactgggtaaaattatttgctttagattatcttgataaatttaactcaggttatgattattatatcgttcctgatacccgtcaagatcatgaaatggatgcggctagggcgatgggtgctacagtaattcatgtagttcgtcctggtcaaaaatccaatgatacacatattacagaagctggattgccaattcgtgatggcgatttagtaattacaaacgatggttctcttgaagaacttttttctaaaattaaaaatacactaaaggtactata

SEQ ID NO: 125 - DNA, 300 bp, synthetic construct
  misc_feature 1..300: note = prom000066
  source 1..300: mol_type = other DNA
aattaaagaaactgaatcagcatttcgtttagcctttgccaaggcgcatttcattaagaaagtaatttcaggtgaaattgttgtgcagggtaaaactcgcaaagaactgaccgaagaactttctaaaattgatatgtattcttcttatgttgataaactagtcggaatgaatatttttcatatgacttccgacgaagcaaagaaacttgctgaagaagctaaagctaagaaagaagaaaacgaatattggaaaactactgatgtagttaccgaatacactaaagatttagaggaaatcaa

SEQ ID NO: 126 - DNA, 300 bp, synthetic construct
  misc_feature 1..300: note = prom000067
  source 1..300: mol_type = other DNA
cggtttgttatttaaacgacgattttgattatctcggtgtttatagtttaagtgacgcatggtttaaacgtaatttacaaaagtcaaatttattttatattgatactactgtaaaatttcagggcaagaaatatttctttactcttatagttgattctgaaacgaagcatgaaaataaacgtattcttagtaaaaagaatatcttaactattgttgatgatctttttgataaatttgtagaaaatcccaattttgaaagtgatttattactagaaaaatttgttaaggaatgtagagaat

SEQ ID NO: 127 - DNA, 300 bp, synthetic construct
  misc_feature 1..300: note = prom000068
  source 1..300: mol_type = other DNA
atgactgctataactccgcaagaatacatggcgtctcttaaagaaaaatataatctttctgcaacagaaacacttttcgatttaccagaaaaccttcaactaaaatttcaggtagaatttcaaaaattaattcacccagaacaaaaacactttactgcagtcgttaaatcaattaatgcagatggaatgacaatttttcaccgacaaatagtactaatttaagcaaggggcttcggccccttatttggagtataatatatcaagagcctaataactcgggctataaactaaggaatatct

SEQ ID NO: 128 - DNA, 300 bp, synthetic construct
  misc_feature 1..300: note = prom000069
  source 1..300: mol_type = other DNA
aacttacttcacttataaatggaaataatcctgacggatcaactgttgaagaacgaggattaactaattctataaaaacgaatgaaactaacattgcggcagtcacacatgaagtaaatacagctaaagacaatatatcctctttacagagcagcgttcaagctctacaagaagcaggttatattcctgaagctccaaaagatggccaagcttacgttcgtaaagacggcgaatgggtactactttctacctttttatcaccagcataacatggggccgcaaggccccaaaggattttaa

SEQ ID NO: 129 - DNA, 300 bp, synthetic construct
  misc_feature 1..300: note = prom000070
  source 1..300: mol_type = other DNA
gtttggtaacacacttgattcactttaccaagattggattacttatccaacgaccccagaagcacgtaccactcgctggacacgtacatggcagaaaactaaaaattcttggtcaagttttgttcaggtatttgacggaggtaaccctcctcaaccttcagatataggagcgatcccatctgataatggaataataggtaatcttactattcgcgatttcttgcgaattggtaatgttcgcattattcctgacccagtgaataaaactgttaaatttgagtggattgaataagaggtatt

SEQ ID NO: 130 - DNA, 300 bp, synthetic construct
  misc_feature 1..300: note = prom000071
  source 1..300: mol_type = other DNA
gttaaatctcttaaggaccgtgaaattatcggtcatgagattggcattatcgcacaggatttacaagaaatattaccggaagctgtgaaatcttcaaaagttggcaatcttgataatccagacgatgttctgacaatttctaactcggcagtgaatgctcttttaattaaggctattcaggaaatgagtgaagaaattaaagaattgaaaacgcctctctttactaaaattgctcgcaaaattagtaaatattttaaattctaacaacaagggggaaatgccccctttggagtataaatt

SEQ ID NO: 131 - DNA, 300 bp, synthetic construct
  misc_feature 1..300: note = prom000072
  source 1..300: mol_type = other DNA
taatgatttagattctagcggaaatccttcacatactgccggtggaacagttggaacaacatcggtgacacttgagaacgcaaatcttcctgcgactaaaactgatgaaaaagttttaattgaagatgaaaacggatcagttattattggaggttgtcaatatgacccagacgaaactggtcctatatatacaaaatatcgtgaagactacgcaacaacaaactcttcacatactcctcctactaatattagtaatattcaaccgtctattactgtataccgttggataaggattgcata

SEQ ID NO: 132 - DNA, 300 bp, synthetic construct
  misc_feature 1..300: note = prom000073
  source 1..300: mol_type = other DNA
ttaaacatattcaagatatgcgagaatttgaagcggggcaataatgcgttattcaattgaagatgcctttaataatgatgaagaatttgaaacagaaattaaattcttaatggaaaagtatcgtctacgacgtcaggatattcgtatcctggcagaccacccttgtggtgaagacgtcctttatattaaagggaagtttgctgggtacatcgacgagtatttttatacaaaagatatgggtatcgatatgtgcatgcgagtggtataaatagatatgtaatttcaacttaggaggcaatc

SEQ ID NO: 133 - DNA, 304 bp, synthetic construct
  misc_feature 1..304: note = prom000074
  source 1..304: mol_type = other DNA
ttattaatgaaagcgtaaaagacctgactgaatctcagaaagaaaaagttattggcctggttgaaggtatggattattctgacgcatttggtactaagttaactgctattgtagaaatggttaaaggttctactaaagaagaagccgctattaccgaaagtataaatacagttgacaatgacgctgccggtcttaattttgtcgccgaagcagttgacactaccacaactcaggtagaacagaattctaatgtaagtttatatgcgaaagtcgcatctcgtttctaatttaaaggttaacacaa

SEQ ID NO: 134 - DNA, 301 bp, synthetic construct
  misc_feature 1..301: note = prom000075
  source 1..301: mol_type = other DNA
cactgttccaggacttaatggaatggcctcaatgatgtctgcctggagtgccgtagcatcccgctataacattactgaagaacagcagaaagaaatatttaaagaactccctttatttgaggatatggttgctggtgattcaggtggcagccctcaagctattgcatcaggaacaactacaggtgcagtcgtaaataaaggtccagaacaaattccttccaagaaaagaaagcgaattaaagtaaatattaataagttgtgatatgatggccttattaatttaaggcctctggagaatact

SEQ ID NO: 135 - DNA, 302 bp, synthetic construct
  misc_feature 1..302: note = prom000076
  source 1..302: mol_type = other DNA
cttcggaaggggaagacattcgcaaagaacgagtgtttaatgaatctcttaaaattatccgttcagcaatggaaaatgtcattcaagagattctgattacacttgaagacggttcccatcatatagtctatattacaaaactagactgggttgatggtaaaatcgtaatggactttgcagttcttgaccaggacagaaaagcagaacttgcaccgcatgttgaaaaatgtgttacaatgcaagtacaacaagcttttaatgaacgaaccaagaaaaaatttaaattcttttaaggagttatc

SEQ ID NO: 136 - DNA, 302 bp, synthetic construct
  misc_feature 1..302: note = prom000077
  source 1..302: mol_type = other DNA
ggtatgaccgccgacgaatttgttgtacaattacgtactactctccggaatgcgatttccgaccaattggctattgcagagtataaagatgaccccactgttgggactaaattacaaataacatatttagataaccaaagacacgtactcccaacgtattcctcttatggtatcacggtttctcaagaaatagtgtctcaggctaagtccgggtacggcacttggaatttactaggtgcgcaaaccattactcttgacaaccatatttcacctacaaccttttattattttgtgagaaccgc

SEQ ID NO: 137 - DNA, 300 bp, synthetic construct
  misc_feature 1..300: note = prom000078
  source 1..300: mol_type = other DNA
accggaagaaaaagcgttcaaatggggactatgcagatcagtttctgttgaaaaagaccatgaattcactattgctgcggcaactgatggaacgccatggaaaactaagcaaatcgccggatttattagcgaccgttgggtagaagatggtgttaagctttataacatcgttttcgctggtcagttcatggtagttcctgaattccaaattaaaaaatatagcccagctgctttcgcataaagttgtttacttctcctctggttttgatattataaccatatcaaccagaggagaataat

SEQ ID NO: 138 - DNA, 300 bp, synthetic construct
  misc_feature 1..300: note = prom000079
  source 1..300: mol_type = other DNA
gtcaatgctcaagtttatctggaattccctatggaaggctggtaatgattaacaaaacatattggtctaatatagacgacggtaatgaaggtatttcgtactattcagtagattggacaggatgtccatcctacttagtgaaacgtatttgtcaagaatttaaaatcgaccagccagcaggtattatcgtgaacagatacattaatggatatcaacctatcgatgaagtaataagaacaactcaagctgagcttgaaaagtttttcaataagtgccaatggataacaggatggattatat

SEQ ID NO: 139 - DNA, 300 bp, synthetic construct
  misc_feature 1..300: note = prom000080
  source 1..300: mol_type = other DNA
aaagaaatgctgcgtatggacggacgtcaggttgatattgatggtgaagactatcagcgtcgtgattcgattgctcagttgcttgaagactggggattgattgttattgaagactctgctcgtgaagacctgtttggcctgactaataacttccgtgttatctcttttaaacagaaagatgactggactttgaaagcaaaatacacaattggtaattaaagcaatgggacttcggtcccatttggagtataataagttcatcaacaaacaaaagacaataactcgtctaaaggaaattaa

SEQ ID NO: 140 - DNA, 300 bp, synthetic construct
  misc_feature 1..300: note = prom000081
  source 1..300: mol_type = other DNA
ggtatgcatgttggtggtgtccaagcacagcaaatgtcatatcataaacacgctggtggatggggcgaatatcaacgtcatgaggcaccattcggagcatccgtatatcaaggttatttgggtactagaaaatacgctgattgggataacgcatcatatttcacaaatgacggcttcgaattaggtggtcaccgcgacgcaacaggcactcttaaccgtgaaggacttatcggatatgagactcgtccatggaatatatctttaaattatattattaaagtccattactaagggtaaaaa

SEQ ID NO: 141 - DNA, 314 bp, synthetic construct
  misc_feature 1..314: note = prom000082
  source 1..314: mol_type = other DNA
agaacgctcaagagaaatctaaactattgggcggttatacttatctgcttaaaaactctgttacagacgaagttaaaccgtccgcaggtttaattgctcaggaagttcaagaagtattacctgaacttgtttctgaagataaagagaccggactgcttcgtttgaactataacggtattattggtttaaatacagctgcaataaacgagcatacagatgaaatcaaggaattgaaatctgaaattaccgaattgaaagcattaattaaatcattgataaaataataaaagggccttcgggccctggaggtttat

SEQ ID NO: 142 - DNA, 300 bp, synthetic construct
  misc_feature 1..300: note = prom000083
  source 1..300: mol_type = other DNA
tccatgcagaattgaatgcaattttgtttgccgctcgtaaaggtaattctattgaaggtgctacgttatatacgacgctttctccatgtcctgattgtactaaagctattacgcagtcaggtattaaaaaggtcgtatacgcagaattatacgaccgatctcctgaaaattgggcagatattctaaaacaagctggtattgaagtaattcagtactctcgtaataatcttcgttctctgaattgggaacaaatccgaaacttttgtggtgaataatatgattttaactgaacaggaaact

SEQ ID NO: 143 - DNA, 299 bp, synthetic construct
  misc_feature 1..299: note = prom000084
  source 1..299: mol_type = other DNA
agtggtattaaaattaagttccgtcaacctaaattatttgatgataaaaatatcgcgttaatgattgcttcatgtattgaggctgtttttgtcaatggagagtccatccctgttgaagatttaactgaagcagaaataaacgacctttatggtcttataacagaagatgacatgatcaatattaaaaatcttttggtgtcgccatcaatttatctggcaacccctattaaatgtcccaaatgcggggcgtcacacgttcacacaatacgaggcctcaaagaattctttgagttactata

SEQ ID NO: 144 - DNA, 300 bp, synthetic construct
  misc_feature 1..300: note = prom000085
  source 1..300: mol_type = other DNA
tgaaatccaaaaaatgaccgaagaaggtaaagattggaaaactgacgatccagatagtaagtactacttgcatcgttacactctccagaagatgatgaaagactatccagaagttgatgttcaagaatcgcgtaatggatacatcattcataaaactgctttagaaactggtatcatctatacctatccataatcataatcataaggggcttcggcccctttcttcattttgaaagcacacaaaacatactcagaaaatgatgtatataatggcatcaactcgataacatgagattgatt

SEQ ID NO: 145 - DNA, 300 bp, synthetic construct
  misc_feature 1..300: note = prom000086
  source 1..300: mol_type = other DNA
ggaatgcatgttggcggtgttcaagcacaacagatgtcataccataaacatgctggtggttggggagaataccaaagacatgaagctccatttggcgcgtccgtttatcaaggatatcttggaactagaaaatatgccgactgggataatgcttcatacttcactaatgatggatttgaattaggtggaccgagagatgcccttggtacacttaatcgtgaaggattaattggttatgaaactagaccatggaatatatcattaaactatattattaaaattcattactaaggattacaa

SEQ ID NO: 146 - DNA, 300 bp, synthetic construct
  misc_feature 1..300: note = prom000087
  source 1..300: mol_type = other DNA
gtttggtaacacacttgattcgctttaccaagattggattacttatccaacgaccccagaagcacgtaccactcgctggacacgtacatggcagaaaaccaaaaactcttggtcaagttttgttcaggtatttgacggaggtaaccctcctcaaccttcagatataggagcgatcccatctgataatggaataataggtaatcttactattcgcgatttcttgcgaattggtaatgttcgcattattcctgacccagtgaataaaactgttaaatttgaatgggttgaataagaggtatt

SEQ ID NO: 147 - DNA, 300 bp, synthetic construct; identical to SEQ ID NO: 126
  misc_feature 1..300: note = prom000088
  source 1..300: mol_type = other DNA

SEQ ID NO: 148 - DNA, 300 bp, synthetic construct; identical to SEQ ID NO: 126
  misc_feature 1..300: note = prom000089
  source 1..300: mol_type = other DNA

SEQ ID NO: 149 - DNA, 300 bp, synthetic construct
  misc_feature 1..300: note = prom000090
  source 1..300: mol_type = other DNA
ttgagcgaaagtactaaagatctgactgagtctcaaaaagaaaaagtctctgctctggtcgaaggtatggattattcagatgcattctcaagtaaattgagtgcaatcgtagaaatggtgaagaaatctaataaagatgaaagcactattactgagagtataaatactcctgatactgaagcagccggactgaatttcgtcactgaagctgtagaagataaagctgcacaaggtgcagaagatattgtaagtgtatatgcgaaagtcgcatctcgtttctaattttaaaggttaacacaa

SEQ ID NO: 150 - DNA, 300 bp, synthetic construct
  misc_feature 1..300: note = prom000091
  source 1..300: mol_type = other DNA
aaattctggaatcagcgtgatattcgtaccagaattgaagcacttctgcttgttcgtgatatgaccacgtgtcctcttccaaaaggaactttagatggattcgttgcgcatgattctattcatgactgtgcgaaagacatcctgatgatgaagtacgctttgcgatatgctatgggtcttgaagatgctccatcagaggaagattgcgatcctctatctcttccaacaaaacgataaaaagttgtttacttcctcggttagttgtggtattataacaccatagctactgaggataataaa

SEQ ID NO: 151 - DNA, 300 bp, synthetic construct
  misc_feature 1..300: note = prom000092
  source 1..300: mol_type = other DNA
gaagagggggaagaagaaagaaaagaacgtctttttaatgaatctcttaagataattaaatctgccatggaaaatgttatccaggagattgtcattaaactagaagatggttctacacacattgtgtatgtgacaaaattagattgggttgatggaaaagtcgtaatggactttgctgttcttgaccaagaaagaaaagctgagttagctcctcatgtagaaaaatgtattacaatgcaactacaagatgcatttaataaaaggtcaaagaaaaagtttaaattcttttaaggagtaagt

SEQ ID NO: 152 - DNA, 300 bp, synthetic construct
  misc_feature 1..300: note = prom000093
  source 1..300: mol_type = other DNA
gattgttaataacttcgaccgcatgtactgggtaaaattatttgctttagattatcttgataaatttaactcaggttatgattattatatcgttcctgatacccgtcaagatcatgaaatggatgcggctagggcgatgggtgctacagtaattcatgtagttcgtcctggtcaaaaatccaatgatacacatattacagaagctggattgccaattcgtgatggcgatttagtaattacaaatgatggttctcttgaagaacttttttctaaaattaaaaatacactaaaggtactata

SEQ ID NO: 153 - DNA, 300 bp, synthetic construct; identical to SEQ ID NO: 118
  misc_feature 1..300: note = prom000094
  source 1..300: mol_type = other DNA

SEQ ID NO: 154 - DNA, 300 bp, synthetic construct
  misc_feature 1..300: note = prom000095
  source 1..300: mol_type = other DNA
taatgatttagattctagcggaaatccttcacacactgccggtggaacagttggaacaacatcggtgacacttgagaacgcaaatcttcctgcgactaaaactgatgaaaaagttttaattgaagatgaaaacggatcagttattattggaggttgtcaatatgacccagacgaaactggtcctatatatacaaaatatcgtgaagactacgcaacaacaaactcttcacatactcctcctactaatattagtaatattcaaccgtctattactgtataccgttggataaggattgcata

SEQ ID NO: 155 - DNA, 300 bp, synthetic construct
  misc_feature 1..300: note = prom000096
  source 1..300: mol_type = other DNA
aaagcgaggtgctggtgttcgccgcggccaattctgactgggcgccacccgaggaactcaagcagttcgcccaggccgcccgcgaagtgatgcgcctgcacccggatgattgcctgtccatggcggccatcgagcggtattttcgcatactgtactggcagaagggcgcggaggagttggatgcgggtaacctgctcggcctgattgagagaggccggctcgatggcctgccctacgagactttggccaccaagttccgcatgatcgacagccttcaactgccggtgatcatcccatttg

SEQ ID NO: 156 - DNA, 300 bp, synthetic construct
  misc_feature 1..300: note = prom000097
  source 1..300: mol_type = other DNA
cagggctggcgccgacgatacggtgggcaagcatctggatatcttcaaccgtcgcgcacggaaggggcagtgcttccatacaccctgcctaggcgtgcgcgagtttccggccagttttcggttgctggaagagggcagtgccgagcctgaagtcgatgcctttctgcgcggcgagcgtgatctgggctggatgctgcatgacattgacttcgccgatggcatgaccccgcacttcttccgtgccctgatgcgcgatgggctgatcgaggtgccggccttcagggcggcagaggacaaggc

SEQ ID NO: 157 - DNA, 300 bp, synthetic construct
  misc_feature 1..300: note = prom000098
  source 1..300: mol_type = other DNA
tcaagacgctgcaccggcagatcttggttggggaaggcgaccccggtctgcaggccttgctccagttcctcgactgttggcagccggagcagttcaagcccccgctgttcagcgaagcaatgctcgacagcaacttagtgttccgcctagacggccaacaacgctatctgcacgagactccggcggccctggcgttgcgtacccggctgttggccgacggcgacagccgcgaggggctgtgcctagtctgcggccaacgtcagccgttggcgcgcctgcatccagcggtcaagggcgtca

SEQ ID NO: 158 - DNA, 300 bp, synthetic construct
  misc_feature 1..300: note = prom000099
  source 1..300: mol_type = other DNA
cgacagggtcaatgccactatccgtgaccgctactacggtgccgcgtccagcacgccagccacggttttcccgatactgctgcgcaacacacaaaaccacttggccaagctgcgcaaggagaagcccggactagcagtgaacctagagcgcgatataggcgaaatcattgacggtatgcagagccaattcccgcgttgcctgcgcctggaggaccagggacgctttgctattggttactaccaacaggcccaggcccgtttcaaccgtggccccgattccgtcgagtaaggagcagaaga

SEQ ID NO: 159 - DNA, 58 bp, synthetic construct
  misc_feature 1..58: note = prom000100
  source 1..58: mol_type = other DNA
ggcattgatatgcatatgagagttgtataaatagatatataattcagaggagacaatc

SEQ ID NO: 160 - DNA, 48 bp, synthetic construct
  misc_feature 1..48: note = prom000101
  source 1..48: mol_type = other DNA
tgcatatgagagttgtataaatagatatataattcagaggagacaatc

SEQ ID NO: 161 - DNA, 1881 bp, synthetic construct
  misc_feature 1..1881: note = Colicin Ib
  source 1..1881: mol_type = other DNA
atgtctgaccctgtacgtattacaaatcccggtgcagaatcgctgggatatgattcagatggccatgaaattatggccgttgatatttatgtaaaccctccacgtgtcgatgtctttcatggtaccccgcctgcatggagttccttcgggaacaaaaccatctggggtggaaacgagtgggtcgatgattccccaacccgaagtgatatcgaaaaaagggacaaggaaatcacagcgtacaaaaacacgctcagcgcgcagcagaaagagaatgagaataagcgtactgaagctggaaaacgcctttctgcggcaattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgatgccctccggctgcatacagagagccggatgctgtttgctgatgctgattctcttcgtatatctccccgcgaggccaggtcgttaatcgaacaggctgaaaaacggcagaaggatgcgcagaacgcagacaagaaggccgctgatatgcttgctgaatacgagcgcagaaaaggtattctggacacgcggttgtcagagctggaaaaaaatggcggggcagcccttgccgttcttgatgcacaacaggcccgtctgctcgggcagcagacacggaatgacagggccatttcagaggcccggaataaactcagttcggtgacggaatcgcttaagacggcccgtaatgcattaaccagagctgaacaacagctgacgcaacagaaaaacacgcctgacggcaaaacgatagtttcccctgaaaaattcccggggcgttcatcaacaaatcattctattgttgtgagtggtgatccgaggtttgccggtacgataaaaatcacaaccagcgcggtcatcgataaccgtgcaaacctgaattatcttctgacccattccggtctggactataaacgcaatattctgaatgaccggaatccggtggtgacagaggatgtggaaggtgacaagaaaatttataatgctgaagttgctgaatgggataagttacggcaacgattgcttgatgccagaaataaaatcacctctgctgaatctgcgataaattcggcgagaaataacgtcagtgccagaacaaatgaacaaaagcatgcaaatgacgctcttaatgccctgttgaaggaaaaagagaatatccgtagccagcttgctgacatcaatcagaaaatagctgaagagaaaagaaaaagggatgaaataaatatggtaaaggatgccataaaactcacctctgatttctacagaacgatatatgatgagttcggtaaacaagcatccgaacttgctaaggagctggcttctgtatctcaagggaaacagattaagagtgtggatgatgcactgaacgcttttgataaattccgtaataatctgaacaagaaatataacatacaagatcgcatggccatttctaaagccctggaagctattaatcaggtccatatggcggagaattttaagctgttcagtaaggcatttggttttaccggaaaagttattgaacgttatgatgttgctgtggagttacaaaaggctgtaaaaacggacaactggcgtccattttttgtaaaacttgaatcactggcagcaggaagagctgcttcagcagttacagcatgggcgttttccgtcatgctgggaacccctgtaggtattctgggttttgcaattattatggcggctgtgagtgcgcttgttaatgataagtttattgagcaggtcaataaacttattggtatctga

SEQ ID NO: 162 - DNA, 1473 bp, synthetic construct
  misc_feature 1..1473: note = Colicin 10
  source 1..1473: mol_type = other DNA
atggataaagtcactgataattctccagatgtggagagcacagaatctactgaggggtcattcccaactgttggggttgatactggcgatacgattacagcgacgcttgcaactggaactgaaaatgttggtggaggcggtggagcatttggtggggccagtgaaagttctgctgcgatacatgcaaccgctaaatggtctaccgcgcagttgaaaaaacatcaggctgaacaggctgcccgtgctgctgcggctgaggcagcattggcaaaagcgaaatctcagcgtgatgccctgactcaacgtctcaaggatattgttaatgacgctttacgtgctaatgccgctcgtagtccatcagtaactgaccttgctcatgccaataatatggcaatgcaggcagaggctgagcgtttgcgccttgcgaaggcagagcaaaaagcccgtgaagaagctgaagcagcagaaaaagcgctccgggaagcagaacgccaacgtgatgagattgcccgccaacaggctgaaaccgcgcatttgttagcaatggcggaggcagcagaggctgagaaaaatcgacaggattctcttgatgaagagcatcgggctgtggaagtggcagagaagaagctggctgaggctaaagctgaactggcgaaggccgaaagcgatgtacagagtaagcaagcgattgtttccagagttgcaggggagcttgaaaacgctcaaaaaagtgttgatgtgaaggttaccggatttcctggatggcgtgatgttcagaaaaaactggagagacaattgcaggataagaagaatgaatattcgtcagtgacgaatgctcttaattctgctgttagcattagagatgctaaaaaaacagaagttcagaatgctgagataaaattaaaagaagctaaggatgctcttgagaagagtcaggtaaaagactctgttgatactatggttgggttttatcaatatataaccgaacaatatggggaaaaatattccagaatagctcaggatttagctgaaaaggcgaagggtagtaaatttaatagtgttgatgaagcacttgctgcatttgaaaagtataaaaatgtactggataagaaattcagtaaggttgatagggatgatatttttaatgctttagagtctattacttatgatgagtgggccaagcatctagaaaagatctctagggctcttaaggttactggatatttgtctttcgggtatgatgtatgggatggtaccctaaagggattaaaaacaggagactggaagcctttatttgtcactctggagaagagcgcggtagatttcggcgtggcaaaaattgtggcattaatgtttagttttattgttggtgcgcctcttggcttctggggaattgcaattatcacaggtattgtttcttcttacataggggatgatgagttgaacaagcttaatgaattactaggtatttaa

SEQ ID NO: 163 - DNA, 1647 bp, synthetic construct
  misc_feature 1..1647: note = Colicin K
  source 1..1647: mol_type = other DNA
atggctaaagaactaagtggatatggaccaactgctggtgagtctatggggggaacaggggcgaatctgaatcagcaaggtggaaataataatagtaatagtggggttcactggggtggtggttctgggcatggtaataatggtggtcagggaaattcaaattcatctggttctacatcaactgtgatgaaaaccggtgagtcatatctgactccgtggggtgatgttgtcattaataatgatggccttccggtgatgaatggtattgttatgacagaagagaattcaactctggttgataatccatttggaggggtctccagagttcttaattctttaattagtgatatgccatcactttttgcagaaagctcaggaaataacaacaataacactgcttcagttaatactgcaccaacgaatgctcaggtcagtgatatggataagagttcgaaggtggtgagtaatgtaattaatgaaaaacaaaagcagaagaataaaattgctacacagattagtgaaaaacagaagaaaatcgaggagatgaaaaaagtattcaagcatcattcttatcatggtatcactgatcttgagagggatgttgatgagttgcaaaaaaaatcaaatcagcttgatgcagatattagcaaacttaattcttataaaaatacattgcaaagtaaaattggtgatgtgaataaacagaaggaagctgaagaaaaggctcgtgaaaatgcagaggtcgctgaacatgaaactcttaatgaagagaaacaggcggtggcggaagcagagaagcgtttagctgaggcgaaagctgaactggcgaaggctgaaagtgatgtacagagtaagcaagctactgtttccagagttgcaggagagcttgaaaacgctcagaagagtgttgacgtgaaggtcaccggatttccgggatggcgtgacgttcagaaaaaactacagcgtcagcttgaagctaagcaggctgagtactccgcagttgaaaatgaacttaagaatgctgttagtttcagggatggtaaggccgctgaggttaaagaggcagagcagaaattaaaagaagctcaggatgctcttgaaaagagccagataaaagatgctgttgatactatggttggattttatcaatatataaccgaacaatatggagaaaaatacgcaaaaattgctcaggatttagcggaaaagtctaaaggaaagaaaattcaaggtgttgatgaagctctggcagcatttgaaaagtataagaatgtactggataagaaattcagtaaggttgatcgggatgctatttttaatgcgttagagtctgttaattatgatgagttatctaagaatttaacgaagatatcaaagtcgcttaaaatcaccagtcgagtatcttttctatatgatgttggttctgattttaaaaatgcaatagaaactggtaactggcgacctttgtttgtcacgctggagaaaagtgcggtcgatgtaggtgtggcaaaaattgtggcattaatgtttagttttattgttggtgtgcctcttggtttctgggggattgcaattgtcacaggtattgtttcttcttacataggggatgatgagttgagcaaacttaatgagttgttagggatttaa

SEQ ID NO: 164 - DNA, 1860 bp, synthetic construct
  misc_feature 1..1860: note = Colicin U
  source 1..1860: mol_type = other DNA
atgcctggatttaattatggtggacatggtgacggtactggctggagttctgaacgtggtgatggtccggctccgggcggtggtatgcagggaaacggtggcggtcatagcggaaataatgacagtggtagcaattctgtgagtcagcagatttctgccattcagaatgatcagaaacttaaacagaaagtggtgaatatgctgattgctgcgagaaaaatgaatcctgatgcgaaaatgattctgggaagtattgctccttctggtgttatgcaggtcaccattgagggggtaacctccacgcaggccaggcagcttggtctgggagggctggtcatgggatataacgcttcaggtgttattggggctgtgggcgaaatagacaccggtcaccgccttaatgcatcaggagcaagtactcccggaagcgaaacctctgtggacagttttgtgaacggacagaaacccgccgaagaatggcatgctgtggcgaaggacagctggaccggggctggtccggtgaatacagggctggttaataacgccatcaaaagtgtccggattatcaaaaaaggctatgtgacgggcgttctcacgccagaagaggtgatgaataaagcagagtacaaagcaatgcgacaggcttttgattcccttccactggcaaaacagggagaggctgtcagacagattgtcgcagcctggagtctggcatatcaggattttccggtaaatctgaaaaaagagatggggcgtgtcactgaacggattgttgatgctattaacctggcgctcattcttaatcagacagagtcccgtttgtcggaatcacaaaagaatgttgatgtagcgaaccagattatcagtgatacagtaaaagctattaatgatgtaaataagaaaatagcagaaaaacgaaatcagcaggtatcactgactgatttgatgaataaaaaacaaaaagaggtcgaagatcttaaaaagatattcaaaaatcactcttatcaccgtattcgtgatgcccagagggaatatgatgatgcccgaaataaatatgctttactggccagtgatattaatgcgctgcaggcacaggtttccggactgacggcaaggaaacagcaggcagaacagaacaaggcagcagcagaaaaagcaaaggcagatgcagcggcgaaagctgcagcagaaaaagcggctgctgaagcgaaagcaaaggctgaagctgaaaaagccagaaaggaagcagaggaaaaagcaaatgatgagaaggctgttctgacaaaagccagtgaaattatcatcagtgttggtgataaggctggggagtatctgggtgataaatataaagttctttctcgtgagatagcagataatatcaaaaactttcagggcaaaacgattcgtagttacgatgaggcgatggcttctgtcaataaactgatggctaatcctgatcttaaaattaatgccgccgacagggatgcaattgtgaatgcctggaaagcatttgatgcagaggatatggggaataagtttgccgcgctgggtaagacgtttaaagccgcagattatgtgatgaaggcaaacaacgtccgggagaagagtatcgaggggtatcagaccggaaactgggggccgctgatgctggagattgagtcctgggtattgagtggtattgcttctgctgtggcgctatcatttttctcagctatctttggtacttttgctatgctgggtgttttttcgactagtttagctgggatcttggctgttattttggctggtttagttggggcgttgattgatgataattttgttgataagttaaataatgaaattattcgtcctgcgtattaa

SEQ ID NO: 165 - protein, 626 aa, synthetic construct
  REGION 1..626: note = Colicin Ib
MSDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLKTARNALTRAEQQLTQQKNTPDGKTIVSPEKFPGRSSTNHSIVVSGDPRFAGTIKITTSAVIDNRANLNYLLTHSGLDYKRNILNDRNPVVTEDVEGDKKIYNAEVAEWDKLRQRLLDARNKITSAESAINSARNNVSARTNEQKHANDALNALLKEKENIRSQLADINQKIAEEKRKRDEINMVKDAIKLTSDFYRTIYDEFGKQASELAKELASVSQGKQIKSVDDALNAFDKFRNNLNKKYNIQDRMAISKALEAINQVHMAENFKLFSKAFGFTGKVIERYDVAVELQKAVKTDNWRPFFVKLESLAAGRAASAVTAWAFSVMLGTPVGILGFAIIMAAVSALVNDKFIEQVNKLIGI

SEQ ID NO: 166 - protein, 490 aa, synthetic construct
  REGION 1..490: note = Colicin 10
MDKVTDNSPDVESTESTEGSFPTVGVDTGDTITATLATGTENVGGGGGAFGGASESSAAIHATAKWSTAQLKKHQAEQAARAAAAEAALAKAKSQRDALTQRLKDIVNDALRANAARSPSVTDLAHANNMAMQAEAERLRLAKAEQKAREEAEAAEKALREAERQRDEIARQQAETAHLLAMAEAAEAEKNRQDSLDEEHRAVEVAEKKLAEAKAELAKAESDVQSKQAIVSRVAGELENAQKSVDVKVTGFPGWRDVQKKLERQLQDKKNEYSSVTNALNSAVSIRDAKKTEVQNAEIKLKEAKDALEKSQVKDSVDTMVGFYQYITEQYGEKYSRIAQDLAEKAKGSKFNSVDEALAAFEKYKNVLDKKFSKVDRDDIFNALESITYDEWAKHLEKISRALKVTGYLSFGYDVWDGTLKGLKTGDWKPLFVTLEKSAVDFGVAKIVALMFSFIVGAPLGFWGIAIITGIVSSYIGDDELNKLNELLGI

SEQ ID NO: 167 - protein, 548 aa, synthetic construct
  REGION 1..548: note = Colicin K
MAKELSGYGPTAGESMGGTGANLNQQGGNNNSNSGVHWGGGSGHGNNGGQGNSNSSGSTSTVMKTGESYLTPWGDVVINNDGLPVMNGIVMTEENSTLVDNPFGGVSRVLNSLISDMPSLFAESSGNNNNNTASVNTAPTNAQVSDMDKSSKVVSNVINEKQKQKNKIATQISEKQKKIEEMKKVFKHHSYHGITDLERDVDELQKKSNQLDADISKLNSYKNTLQSKIGDVNKQKEAEEKARENAEVAEHETLNEEKQAVAEAEKRLAEAKAELAKAESDVQSKQATVSRVAGELENAQKSVDVKVTGFPGWRDVQKKLQRQLEAKQAEYSAVENELKNAVSFRDGKAAEVKEAEQKLKEAQDALEKSQIKDAVDTMVGFYQYITEQYGEKYAKIAQDLAEKSKGKKIQGVDEALAAFEKYKNVLDKKFSKVDRDAIFNALESVNYDELSKNLTKISKSLKITSRVSFLYDVGSDFKNAIETGNWRPLFVTLEKSAVDVGVAKIVALMFSFIVGVPLGFWGIAIVTGIVSSYIGDDELSKLNELLGI

SEQ ID NO: 168 - protein, 619 aa, synthetic construct
  REGION 1..619: note = Colicin U
MPGFNYGGHGDGTGWSSERGDGPAPGGGMQGNGGGHSGNNDSGSNSVSQQISAIQNDQKLKQKVVNMLIAARKMNPDAKMILGSIAPSGVMQVTIEGVTSTQARQLGLGGLVMGYNASGVIGAVGEIDTGHRLNASGASTPGSETSVDSFVNGQKPAEEWHAVAKDSWTGAGPVNTGLVNNAIKSVRIIKKGYVTGVLTPEEVMNKAEYKAMRQAFDSLPLAKQGEAVRQIVAAWSLAYQDFPVNLKKEMGRVTERIVDAINLALILNQTESRLSESQKNVDVANQIISDTVKAINDVNKKIAEKRNQQVSLTDLMNKKQKEVEDLKKIFKNHSYHRIRDAQREYDDARNKYALLASDINALQAQVSGLTARKQQAEQNKAAAEKAKADAAAKAAAEKAAAEAKAKAEAEKARKEAEEKANDEKAVLTKASEIIISVGDKAGEYLGDKYKVLSREIADNIKNFQGKTIRSYDEAMASVNKLMANPDLKINAADRDAIVNAWKAFDAEDMGNKFAALGKTFKAADYVMKANNVREKSIEGYQTGNWGPLMLEIESWVLSGIASAVALSFFSAIFGTFAMLGVFSTSLAGILAVILAGLVGALIDDNFVDKLNNEIIRPAY

SEQ ID NO: 169 - DNA, 1362 bp, synthetic construct
  misc_feature 1..1362: note = LBP-EC01-leuO, Type I-E and Type I-F ftsA targeting CRISPR RNA sequences
  source 1..1362: mol_type = other DNA
gataattagtgctgcgggtaggtaaggataaaaaagggtggcagcaggagattgagatggttttgctttattaacaacgggctaaacgtgtagtatttgagctagcgaattcgagctcggtaccacctcgagttccccgcgccagcggggataaaccgctgaagtagaaaaacgtgttacagcatcagtcgagttccccgcgccagcggggataaaccgggagagagtgagcgatcctccgttaacatagttcactgccgtacaggcagcttagaaagtattattcgacggcggtgggattgcttcacgttcactgccgtacaggcagcttagaaaatcaaaattgctgtctgccaggtgatcgctttgacagctagctcagtcctaggtactgtgctagcataaaggaggtaaataatgccagaggtacaaacagatcatccagagacagcggagttaagcaaaccacagctacgcatggtcgatctcaacttattaaccgttttcgatgccgtgatgcaggagcaaaacattactcgtgccgctcatgttctgggaatgtcgcaacctgcggtcagtaacgctgttgcacgcctgaaggtgatgtttaatgacgagctttttgttcgttatggccgtggtattcaaccgactgctcgcgcatttcaactttttggttcagttcgtcaggcattgcaactagtacaaaatgaattgcctggttcaggttttgaacccgcgagcagtgaacgtgtatttcatctttgtgtttgcagcccgttagacagcattctgacctcgcagatttataatcacattgagcagattgcgccaaatatacatgttatgttcaagtcgtcattaaatcagaacactgaacatcagctgcgttatcaggaaacggagtttgtgattagttatgaggacttccatcgtcctgaatttaccagcgtaccattatttaaagatgaaatggtgctggtagccagcaaaaatcatccaacaattaagggcccgttactgaaacatgatgtttataacgaacaacatgcggcggtttcgctcgatcgtttcgcgtcatttagtcaaccttggtatgacacggtagataagcaagccagtatcgcgtatcagggcatggcaatgatgagcgtacttagcgtggtgtcgcaaacgcatttggtcgctattgcgccgcgttggctggctgaagagttcgctgaatccttagaattacaggtattaccgctgccgttaaaacaaaacagcagaacctgttatctctcctggcatgaagctgccgggcgcgataaaggccatcagtggatggaagagcaattagtctcaatttgcaaacgctaa

SEQ ID NO: 170 - DNA, 300 bp, synthetic construct
  misc_feature 1..300: note = prom000106
  source 1..300: mol_type = other DNA
actgcaaccatgaaaattggttctgtactgaaagctgacaacacagaagctactgctgctggtgaagctgttaaagttctgctgtggactgacgcactgttcgggatcgatgatgttgcagtaggtgacactttcaccgcagttgttggtgtacgtgacctgactctcaaccgctatgcagtttactacaagagtggtgatctgattgatgacgctggcgttgcagcactggaaactcacgatctgaaactgactgaaaaagttgttttcacttcttaatttttaataggagatttatac

SEQ ID NO: 171 - DNA, 1881 bp, synthetic construct
  misc_feature 1..1881: note = Colicin Ia
  source 1..1881: mol_type = other DNA
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtatttaa

SEQ ID NO: 172 - DNA, 1569 bp, synthetic construct
  misc_feature 1..1569: note = Colicin E1
  source 1..1569: mol_type = other DNA
atggaaaccgcggtagcgtactataaagatggtgttccttatgatgataagggacaggtaattattactcttttgaatggtactcctgacgggagtggctctggcggcggaggtggaaaaggaggcagtaaaagtgaaagttctgcagctattcatgcaactgctaaatggtctactgctcaattaaagaaaacacaggcagagcaggctgcccgggcaaaagctgcagcggaagcacaggcgaaagcaaaggcaaacagggatgcgctgactcagcgcctgaaggatatcgtgaatgaggctcttcgtcacaatgcctcacgtacgccttcagcaacagagcttgctcatgctaataatgcagctatgcaggcggaagacgagcgtttgcgccttgcgaaagcagaagaaaaagcccgtaaagaagcggaagcagcagaaaaggcttttcaggaagcagaacaacgacgtaaagagattgaacgggagaaggctgaaacagaacgccagttgaaactggctgaagctgaagagaaacgactggctgcattgagtgaagaagctaaagctgttgagatcgcccaaaaaaaactttctgctgcacaatctgaagtggtgaaaatggatggagagattaagactctcaattctcgtttaagctccagtatccatgcccgtgatgcagaaatgaaaacgctcgctggaaaacgaaatgaactggctcaggcatccgctaaatataaagaactggatgagctggtcaaaaaactatcaccaagagccaatgatccgcttcagaaccgtcctttttttgaagcaaccagacgacgggttggggccggtaagattagagaagaaaaacaaaaacaggtaacagcatcagaaacacgtattaaccggataaatgctgatataactcagatccagaaggctatttctcaggtcagtaataatcgtaatgccggtatcgctcgtgttcatgaagctgaagaaaatttgaaaaaagcacagaataatctccttaattcacagattaaggatgctgttgatgcaacagttagcttttatcaaacgctgactgaaaaatatggtgaaaaatattcgaaaatggcacaggaacttgctgataagtctaaaggtaagaaaatcggcaatgtgaatgaagctctcgctgcttttgaaaaatacaaggatgttttaaataagaaattcagcaaagccgatcgtgatgctatttttaatgcgttggcatcggtgaagtatgatgactgggctaaacatttagatcagtttgccaagtacttgaagattacggggcatgtttcttttggatatgatgtggtatctgatatcctaaaaattaaggatacaggtgactggaagccactatttcttacattagagaagaaagctgcagatgcaggggtgagttatgttgttgctttactttttagcttgcttgctggaactacattaggtatttggggtattgctattgttacaggaattctatgctcctatattgataagaataaacttaatactataaatgaggtgttagggatttaa

SEQ ID NO: 173 - DNA, 2094 bp, synthetic construct
  misc_feature 1..2094: note = Colicin D
  source 1..2094: mol_type = other DNA
atgagtgattacgaaggtagtggtccgacagaaggtattgattatgggcactcgatggtcgtgtggccgtcaacaggactgatttcgggtggtgatgtgaaaccaggaggctcatcaggtatcgctccatccatgcctccgggatggggggattacagcccacagggtatcgcacttgtacaaagtgttctttttccgggaattattcgccggattattctggataaggaacttgaagagggagactggtcgggatggtctgtcagtgtgcatagcccttggggaaacgagaaagtttccgctgcacgaacagttcttgagaatggtttacgtggtggtttgccagaaccgtctcgcccggctgctgtttcttttgcccgtctggagcctgcttccggaaatgagcaaaaaattattcgtcttatggttacacagcaactggaacaggtaacggatattccggccagccagttaccagcagcgggtaataatgtaccggtgaaatatcgtctgatggaccttatgcagaacggtacgcaatatatggctattatcggaggtattccgatgacagtgccggtagttgatgccgttccagttccggaccggagtcgtccgggaacaaatattaaagatgtttacagtgcccctgtatcaccaaatctaccggacctggtattaagtgtgggtcagatgaatactccggttctgtctaatcccgaaattcaggaagaaggagttattgctgagacaggtaattatgttgaggctggttatacgatgtccagtaataatcatgatgtcattgttcgttttcctgaaggcagtgatgtttctccgttatatatttcaactgtagagattctggacagtaatggtctgagtcagcgtcaggaagccgaaaataaagcaaaggatgatttcagagtcaagaaagaagaagcggtagcccgggctgaagctgaaaaagcaaaagccgagttatttagtaaagcaggtgtgaaccagcctcctgtatatacacaggaaatgatggaaagggctaattcagtaatgaatgagcagggagctcttgttctgaataacactgccagttctgtacaactggcgatgaccggaactggtgtctggactgctgctggtgatattgcaggcaacataagcaagtttttcagtaatgctctggagaaagtaaccatacccgaagtgagtcccctgcttatgcggatttctcttggtgctctgtggtttcattcagaagaagctggagcaggaagtgatatcgtgccggggcgaaatctggaggcgatgttttcgctgagtgctcagatgttggctggacagggcgtggtcattgaacctggtgcgacgagtgtaaatctgcctgttcgtggacaattgataaacagtaacgggcaattagctctggatttactgaaaacagggaatgaaagtatcccggctgcggttcctgttcttaatgctgtccgtgatacagcaacaggactggataaaatcacgttaccagcagtagtaggcgcaccttcccggacgattctggttaatccggtaccacaaccttcggtgccaacagatacaggtaatcatcaacccgttccggttacaccagtgcatacaggaacggaagtaaaatcggtcgaaatgccagtaacgacgattactcctgtttctgatgttggtggactacgagattttatttactggcgtcctgatgctgctgggactggtgttgaagctgtttatgtgatgcttaatgatcctctggattcagggcgattttcgcgtaaacaacttgataaaaaatataaacatgctggtgattttggtattagtgatacaaaaaagaatcgtgaaactcttactaaattcagggatgctattgaggagcatttatcggataaggatacagtagagaaaggaacataccgaagagaaaaaggttcaaaagtttattttaatcctaatacgatgaatgtggttataattaagtcaaatggtgagttcttatctgggtggaaaataaatccagatgcggataatggtcgaatttatttagagacaggtgaactatga

SEQ ID NO: 174 - DNA, 1746 bp, synthetic construct
  misc_feature 1..1746: note = Colicin E2
  source 1..1746: mol_type = other DNA
atgagcggtggcgatggacgcggccataacacgggcgcgcatagcacaagtggtaacattaatggtggcccgaccgggcttggtgtaggtggtggtgcttctgatggctccggatggagttcggaaaataacccgtggggtggtggttccggtagcggcattcactggggtggtggttccggtcatggtaatggcggggggaatggtaattccggtggtggttcgggaacaggcggtaatctgtcagcagtagctgcgccagtggcatttggttttccggcactttccactccaggagctggcggtctggcggtcagtatttcagcgggagcattatcggcagctattgctgatattatggctgccctgaaaggaccgtttaaatttggtctttggggggtggctttatatggtgtattgccatcacaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaataacagtacgccagaagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccggacttaaagctcagcgggcgcagacggatgtaaataataagcaggctgcatttgatgctgctgcaaaagagaagtcagatgctgatgctgcattaagtgccgcgcaggagcgccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaaactttgacgatttccggaagaaattctgggaagaagtgtcaaaagatcccgatcttagtaagcaatttaaaggcagtaataagacgaacattcaaaagggaaaagcaccttttgcaaggaagaaagaccaagtaggtggtagggaacgctttgaattacatcatgataaaccaatcagtcaggatggtggtgtctatgatatgaataatatcagagtgaccacacctaagcgacatattgatattcatcggggtaagtaa

SEQ ID NO: 175 - DNA, 1656 bp, synthetic construct
  misc_feature 1..1656: note = Colicin E3
  source 1..1656: mol_type = other DNA
atgagcggtggcgatggacgcggccataacacgggcgcgcatagcacaagtggtaacattaatggtggcccgaccgggcttggtgtaggtggtggtgcttctgatggctccggatggagttcggaaaataacccgtggggtggtggttccggtagcggcattcactggggtggtggttccggtcatggtaatggcggggggaatggtaattccggtggtggttcgggaacaggcggtaatctgtcagcagtagctgcgccagtggcatttggttttccggcactttccactccaggagctggcggtctggcggtcagtatttcagcgggagcattatcggcagctattgctgatattatggctgccctgaaaggaccgtttaaatttggtctttggggggtggctttatatggtgtattgccatcacaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgagcgacagaatatttcggttgtttcaggtgttccgatgagtgttccggtggttgatgcaaaacctaccgaacgtccgggtgtttttacggcatcaattccaggtgcacctgttctgaatatttcagttaataacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagccctgaccaggtaaaacaacgtcaagatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagcccagcgggcgcagacggatgtaaataataagcaggctgcatttgatgctgctgcaaaagagaagtcagatgctgatgctgcattgagttctgctatggaaagcaggaagaagaaagaagataagaaaaggagtgctgaaaataatttaaacgatgaaaagaataagcccagaaaaggttttaaagattacgggcatgattatcatccagctccgaaaactgagaatattaaagggcttggtgatcttaagcctgggataccaaaaacaccaaagcagaatggtggtggaaaacgcaagcgctggactggagataaagggcgtaagatttatgagtgggattctcagcatggtgagcttgaggggtatcgtgccagtgatggtcagcatcttggctcatttgaccctaaaacaggcaatcagttgaaaggtccagatccgaaacgaaatatcaagaaatatctttga

SEQ ID NO: 176 - DNA, 1536 bp, synthetic construct
  misc_feature 1..1536: note = Colicin B
  source 1..1536: mol_type = other DNA
atgagtgataatgaaggtagtgtaccgacagaaggtattgattacggggacacaatggttgtgtggccgtcaacaggacgaattccgggcggtgatgtgaaacccggaggctcatcaggtctcgctccatccatgcctccgggatggggggattacagcccacaaggtatcgcacttgtacaaagtgttctttttcctggaattattcgccggattattcttgataaagaacttgaagagggagactggtcgggatggtctgtcagtgtgcatagcccctggggaaacgagaaagtttccgctgcacgaacagttcttgaaaatggtttacgtggtggtttgccagaaccgtctcgcccggctgctgtttcttttgcccgtctggagcctgcttccggaaatgagcaaaaaattattcgtcttatggttacacagcaactggagcaggtaacggatatccctgccagccagttaccagcagcgggtaataatgtaccggtaaaatatcgtctgacggaccttatgcagaatggtacacaatatatggctattatcggaggtattccgatgacagtgccagtagtggatgccgttccagttccggaccggagtcgtccgggaaccaatattaaagatgtttacagtgcccctgtatcaccaaatctaccggacctggtattaagtgtgggtcagatgaatactccagttcggtctaatcccgaaatccaggaagatggcgttatttctgagacagggaattatgttgaggctggttatacgatgtccagtaataatcatgatgtcattgtccgttttcctgaaggcagtggagtttctccgctatatatttcagccgtggagattctggacagtaatagtttaagccagcgccaggaagccgaaaataacgcaaaggatgacttcagagtcaagaaagaacaagaaaatgacgagaagacggtcctgacaaaaaccagcgaggtcatcattagtgtcggtgacaaagtcggggaatatcttggagataaatacaaggcgctttcccgtgaaattgcagagaatataaataattttcagggaaaaacgattcgtagttatgatgatgcaatgtcttccattaataagttaatggctaaccccagccttaaaataaatgcaacggacaaagaagccattgtgaatgcgtggaaagcatttaatgctgaggatatggggaataaatttgctgcgttgggtaaaacgttcaaagcagcagattatgcaataaaggcaaacaacatcagggagaagagtattgagggttaccagactggtaactgggggccattaatgctggaagtcgagtcctgggttatcagtgggatggcatctgctgtagctcttagtttgttttctttgacattaggctcggcccttatagcctttggtctttcggccacagttgttggttttgttggcgtagttattgcaggtgctattggtgcatttatcgatgataaatttgttgatgagttgaatcacaagatcataaaataa

SEQ ID NO: 177 - protein, 626 aa, synthetic construct
  REGION 1..626: note = Colicin Ia
MSDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGI

SEQ ID NO: 178 - protein, 522 aa, synthetic construct
  REGION 1..522: note = Colicin E1
METAVAYYKDGVPYDDKGQVIITLLNGTPDGSGSGGGGGKGGSKSESSAAIHATAKWSTAQLKKTQAEQAARAKAAAEAQAKAKANRDALTQRLKDIVNEALRHNASRTPSATELAHANNAAMQAEDERLRLAKAEEKARKEAEAAEKAFQEAEQRRKEIEREKAETERQLKLAEAEEKRLAALSEEAKAVEIAQKKLSAAQSEVVKMDGEIKTLNSRLSSSIHARDAEMKTLAGKRNELAQASAKYKELDELVKKLSPRANDPLQNRPFFEATRRRVGAGKIREEKQKQVTASETRINRINADITQIQKAISQVSNNRNAGIARVHEAEENLKKAQNNLLNSQIKDAVDATVSFYQTLTEKYGEKYSKMAQELADKSKGKKIGNVNEALAAFEKYKDVLNKKFSKADRDAIFNALASVKYDDWAKHLDQFAKYLKITGHVSFGYDVVSDILKIKDTGDWKPLFLTLEKKAADAGVSYVVALLFSLLAGTTLGIWGIAIVTGILCSYIDKNKLNTINEVLGI

SEQ ID NO: 179 - protein, 697 aa, synthetic construct
  REGION 1..697: note = Colicin D
MSDYEGSGPTEGIDYGHSMVVWPSTGLISGGDVKPGGSSGIAPSMPPGWGDYSPQGIALVQSVLFPGIIRRIILDKELEEGDWSGWSVSVHSPWGNEKVSAARTVLENGLRGGLPEPSRPAAVSFARLEPASGNEQKIIRLMVTQQLEQVTDIPASQLPAAGNNVPVKYRLMDLMQNGTQYMAIIGGIPMTVPVVDAVPVPDRSRPGTNIKDVYSAPVSPNLPDLVLSVGQMNTPVLSNPEIQEEGVIAETGNYVEAGYTMSSNNHDVIVRFPEGSDVSPLYISTVEILDSNGLSQRQEAENKAKDDFRVKKEEAVARAEAEKAKAELFSKAGVNQPPVYTQEMMERANSVMNEQGALVLNNTASSVQLAMTGTGVWTAAGDIAGNISKFFSNALEKVTIPEVSPLLMRISLGALWFHSEEAGAGSDIVPGRNLEAMFSLSAQMLAGQGVVIEPGATSVNLPVRGQLINSNGQLALDLLKTGNESIPAAVPVLNAVRDTATGLDKITLPAVVGAPSRTILVNPVPQPSVPTDTGNHQPVPVTPVHTGTEVKSVEMPVTTITPVSDVGGLRDFIYWRPDAAGTGVEAVYVMLNDPLDSGRFSRKQLDKKYKHAGDFGISDTKKNRETLTKFRDAIEEHLSDKDTVEKGTYRREKGSKVYFNPNTMNVVIIKSNGEFLSGWKINPDADNGRIYLETGEL

SEQ ID NO: 180 - protein, 581 aa, synthetic construct
  REGION 1..581: note = Colicin E2
MSGGDGRGHNTGAHSTSGNINGGPTGLGVGGGASDGSGWSSENNPWGGGSGSGIHWGGGSGHGNGGGNGNSGGGSGTGGNLSAVAAPVAFGFPALSTPGAGGLAVSISAGALSAAIADIMAALKGPFKFGLWGVALYGVLPSQIAKDDPNMMSKIVTSLPADDITESPVSSLPLDKATVNVNVRVVDDVKDERQNISVVSGVPMSVPVVDAKPTERPGVFTASIPGAPVLNISVNNSTPEVQTLSPGVTNNTDKDVRPAGFTQGGNTRDAVIRFPKDSGHNAVYVSVSDVLSPDQVKQRQDEENRRQQEWDATHPVEAAERNYERARAELNQANEDVARNQERQAKAVQVYNSRKSELDAANKTLADAIAEIKQFNRFAHDPMAGGHRMWQMAGLKAQRAQTDVNNKQAAFDAAAKEKSDADAALSAAQERRKQKENKEKDAKDKLDKESKRNKPGKATGKGKPVGDKWLDDAGKDSGAPIPDRIADKLRDKEFKNFDDFRKKFWEEVSKDPDLSKQFKGSNKTNIQKGKAPFARKKDQVGGRERFELHHDKPISQDGGVYDMNNIRVTTPKRHIDIHRGK

SEQ ID NO: 181 - protein, 551 aa, synthetic construct
  REGION 1..551: note = Colicin E3
MSGGDGRGHNTGAHSTSGNINGGPTGLGVGGGASDGSGWSSENNPWGGGSGSGIHWGGGSGHGNGGGNGNSGGGSGTGGNLSAVAAPVAFGFPALSTPGAGGLAVSISAGALSAAIADIMAALKGPFKFGLWGVALYGVLPSQIAKDDPNMMSKIVTSLPADDITESPVSSLPLDKATVNVNVRVVDDVKDERQNISVVSGVPMSVPVVDAKPTERPGVFTASIPGAPVLNISVNNSTPAVQTLSPGVTNNTDKDVRPAGFTQGGNTRDAVIRFPKDSGHNAVYVSVSDVLSPDQVKQRQDEENRRQQEWDATHPVEAAERNYERARAELNQANEDVARNQERQAKAVQVYNSRKSELDAANKTLADAIAEIKQFNRFAHDPMAGGHRMWQMAGLKAQRAQTDVNNKQAAFDAAAKEKSDADAALSSAMESRKKKEDKKRSAENNLNDEKNKPRKGFKDYGHDYHPAPKTENIKGLGDLKPGIPKTPKQNGGGKRKRWTGDKGRKIYEWDSQHGELEGYRASDGQHLGSFDPKTGNQLKGPDPKRNIKKYL

SEQ ID NO: 182 - protein, 511 aa, synthetic construct
  REGION 1..511: note = Colicin B
MSDNEGSVPTEGIDYGDTMVVWPSTGRIPGGDVKPGGSSGLAPSMPPGWGDYSPQGIALVQSVLFPGIIRRIILDKELEEGDWSGWSVSVHSPWGNEKVSAARTVLENGLRGGLPEPSRPAAVSFARLEPASGNEQKIIRLMVTQQLEQVTDIPASQLPAAGNNVPVKYRLTDLMQNGTQYMAIIGGIPMTVPVVDAVPVPDRSRPGTNIKDVYSAPVSPNLPDLVLSVGQMNTPVRSNPEIQEDGVISETGNYVEAGYTMSSNNHDVIVRFPEGSGVSPLYISAVEILDSNSLSQRQEAENNAKDDFRVKKEQENDEKTVLTKTSEVIISVGDKVGEYLGDKYKALSREIAENINNFQGKTIRSYDDAMSSINKLMANPSLKINATDKEAIVNAWKAFNAEDMGNKFAALGKTFKAADYAIKANNIREKSIEGYQTGNWGPLMLEVESWVISGMASAVALSLFSLTLGSALIAFGLSATVVGFVGVVIAGAIGAFIDDKFVDELNHKIIK

SEQ ID NO: 183 - DNA, 6577 bp, synthetic construct
  misc_feature 1..6577: note = Deletion
  source 1..6577: mol_type = other DNA
tattacgagatacacgtccgaaccaacgccatccattactaattgcacgagaaccagtgtgccatgtttgccaatcaaactgaagaagagtacggtctggagcttcccatttctctagttcacctttttcaattttttcgaggacttctttgtgccactggcgatagataagttcgccatcaggaatctgactgaactctgcagttcctgttgcaaaatgagtaggacacacatcagcgttaacaagaccaagaatatgttcagaatggtaacgagggttatcatagtcaggttgtcctgcagtaataaagtgttggccgactggaatatcagggcgaggaacatcgtcatggtgatatccaggaatagcaggataccaaccaggcatcagcatgtggactcgtgaatcaaatacaacatgttgattagtccaatcaattgggagattggcaataaagctacgagtaataggaccaccattatcccaagcaaaactcaaatcacagttaaagaacattggttcatttttgatttggtcattttgtacgttatgagcaaaaccacctactgcacgagcttgactattaaacactttagaactattcatattattttcctcgacattctttaatgaaattttctagattggtagattcaatagcttttttacgagcttctgcgtgttcttcttcagacaatttacatttataattaacccagacgttatggtcaaaactagtcgtggcatataataacaatgctccaacgaatagcaaaaacggcgataagaatacaaaaccaggaacaccatccatatcgtaagccttttgtaatactgcaacagaaagagctacgtaaaaaagtgatacaaatatagttaaaagaccaattcggttggtttctgttaatttcataatattctcctcatgttttaataggaccactataacatagtcctataattgtgtaaacagttttgtgaagttatttgaaaggttttaccatatggaaattttgtttgaaacgtttgacataatcgtcgtgtcctaagctttggtacatacttacaactttgctgtagttttcccaggcgtactcacggaaccaaccggaggtgatgcttgtgcagactttttccaatgccatcataaaactggcttcagggttaatttcaaagttattaggaatctgagaacgttctaatgctagtacacatgattcttcgtatacaccggccaatttgaccatttctggtaatgattcgaacttctcgcgtgaggtcatgacttcagaaccatctttcatataaaacgtataagccggacggtctgttagtgcaatggcttcgtgaatagtatcatggtcatacgtatagatgtcatctttaaagaatgcgcttttagaagtgtcaagaactgggtgagcatagttcaaggtttctttctggcgtttcagcataatttcagtaagttcatcgttcagtgtaacacctttaccacgaagatactgaatatggttcatagtctttaaaaagaatggattatttttcttgaaacgatgagacatcttaattgcaagacacatctcaggagttgcccagtaaaacccgttcaaacggtctttagtcaaatttttcttggcgtattttaaaaggtcataagaagacgtgaaatcttcaggagtgagttcaccgactaattcacggacattagtgattgccggaacaatgtatgcttcaaaatgagtttctcgtccattatggaagcatttgaaaacttggacaccagggttatcttggtgaacatatacacccatcatttgatttttgaatacattccaagaaccttggtccgcaataaagtcccaatcactatttttgatagcgctagaatcaataaggccgtgatagtgaagtgcacgagaaccaataactaacatcataataatttcctcgaatttagattacttaaatttcgcgacatgcgatattatattcatcccagaggtcatcgtgtgaagcctctaccaatggaacgattgtaccgtttatgtcttcattataaggacaaattgtacagtgcgcatcttcgaatcggatgcctttgtgttcagattttttataacgcatatcgaatatctttgcaaacttttcacgactaatagttttagcaactactttggttccgatactttcgtaataaatcattgtggactccatttaatttgataggactactataacatagccctatcttgttgtaaactgttttagcaagaataactagatgatacccagccttgaacagtgatactattgtgggataatgagatcgcattctggagattcagacctgcgttatcttcaatccataatgtaacttcttcgcgttcttctgttgcaatttggaacaggttttccagagcagctactgccgtacgtacagcacaatctaattcacgagacatattggttccttagcaagtgtgagaagatgcaacccaaccaccatcttcaaggctcaagttgtagtcgtattggtaaacgtcggcccattcagcataaccgttttttgtacaacgttcaagatcggctttaagatgtccgggtgaatgataggttccacccatgccataagctggatacaagttgaatgttgtgttttgtttattagcaatttctttggcagcctcaatagctgcatagatttttgctacagcttcagccagttcggtttgttctttagacataatgattccttagcacagcgcagaagaggaaacccaagcaccttcagtaataacgtaaccattaccttcacgcttaacatcgccaccattattttcaatttcatttttcatccactgggatacgatttcacccttaggataataagtacgaccgctaccataatcacctacagaaaacgatactgcatattggtctgcgatctttgttgcttcttcttcgatacgttcagcttgattcagaagtttataaattgcatctgaagcagattcggcatcattaaactctggtactttaatttcaataatcttactcattttctacattccttaataaagttttcgattgtttcattttcacgtctagctaggagttcattataactaatcatttgagaagcgtaaacttttttatttttcttgtcatggtttataaaatcgtagtaaaaatagactacagtacaaaatgatgcgatggatgcaaaggcttgggtataccgtaagtcctgccattgatctgtgaatccaccagtgttccatataaatcctaacacaacgccgtcaataataaatccaactaatccaaaggcaaatcccatgccaatactcataataaaaattagaatgttctcaccaagagaaggccatacggttaagagttgaggcttttgcatcttatttccttaacgaattccatagcttcttcatattcatactttttaacagcttcatcatactgagcttgggcttttgcgcactgaattttccattctcttaatcgggctctgtgatgacgtccttggtaccaatatcccacccagttaataggcattaataaaaacggtacaataagtggtgcgacgaacattaatgaaaatgctgcatcagacgatataacttcactagaatctaaaacggctcctataatcataaagattaaaaatgctaaagctaaaatagggccaaatagtacttcatttgggattaatttacgcttgtactcatacgcccagggcttacttggcatgtatagggttggctttgacatattctctacattccataataaattgctcaaggattaaattgttagtgaaattaggactaacaaccatagtttcataaagcgcatcaatagcacttaaagcatcaactcttttaagcaaaggcttgccttcatacatagtttcagaatcgatgattaacgtaaagtaatattttttattatcgactagaacttctgtatcgatataaaacatattagacctcagaagcttatctctaaaaccataataatcttcagagcgttcgctgatatattccaattcagtgtttaaacaacaaacagaccacgtaggagcaacgaaatcagtgaatctaatatcatggtctgcaactagtttagcaatagctccgacatcattaatcatgatacctggggctaataaagattttcggttatttgcatattcagttgtctcataagaagctgaccagtaccatttacgcccggttgcaacatatcgtaatgaatcaactaaagacgtaggtggatttaccttatctcggaactcttcgaacattgagtcaatatcattattttggtttcgcatgttaactcctttattatccaaaaaggaggaccggagtcctccgtattattttatttcaaagagttaatatattcttgtacatcagcagaagtggtttcaacaccgactggagtgccattgaatgtttcaacacgagtcagagtgtcttcgatatcaaccttagtcagtgcagcaatttcgattacatcatcagccgtagagataccaagagcattggctgctcgagtttcgcggatgtattcaagtttgattgccagattctgacgagcatcatcaagttcaaccaccttacgagcaatttcaattcgcatagcggcataaccgtcagcttttgtattaagttgttcggcagttcgacgatacagaagaccaagcttagcatgcatggtaacatcttgtccttcagccaggagcttacgaatttcacgttccttagaagcggcttgcttgttcttttcattaacaagttcgcgaatacgtttttcttcattaatagatttaacagaagccgtttttagttctttgatttgggtaatcaacttatctgcagctgcgttatactgttcttcgacagtcagatttttagccatagcagtacccagtttagtgcggatgaattcaacaagtttcttcagtgtgttcatattattacctttcaattggtggtttattatccaacgagagcattatactctgctctcaagagtttgtaaattattttttaatcaaattgacataaaacttggcgtcttcagggtgcatgccttcacagtattcttcaaccatgaacttcttaacagcttcataaggaccgctcagattaagctcgattgtccaaaacttggaatcttggactgaatcgatacgaacttcagggtgacggttgcgaataacttcttcagtgtattcataatcaacgacatcaatactaactgtagccattttgtttctcctctgtagttgataagtctatagtatcaccatccttggtgtttgtacatcattatttttaatttttccaacttaacaacttgtagtgtccttcattcgtacgaggtttatcccaagaaatactaatacatgattcgctgtcaatagaagtcgccgggtttttctctacagtaagaccttcgctttccagccattcaatagtatttgcgcataaatctgaagaacacctcatcggaaactggcaagatttttcacctttagatgcagcgttatacatcttttctaaaatacgcttttgcacgtttttaaaaatagtttcagcagattcttcagattttttattcagttcagtataaagactcataatattctcctcattaatagggctcataatatctcaatcataagcccatgtacgcatttatttcatattatcgaaatattcttcagcgatttcgacattatcatggtaaactttagaagacagtttaacataaccttctgctgcgaacatattaatcacaacctttacagtataccattgtccgtcttcattacccattactgcgtaagtttcaaacatcggatggtcaggtccaataactttaatatcattcaccgtacgtccgaaatcttctggaacgcatttcataaagaagttgaacagttcaccgtaattatccatttcattctccaaggtttttctgtatcggtagttgatagttgtatagtaccacggaagaacagggatgtaaacagttttgtgaaaaaaatttttaaaaagttttgtggaattctagggcagggaggggaaatcaaaggataggataatatattataaagggcagaaactaaatgatgcctagagaggtcgggaaaggcctagataccaaaaagccctatcatttagatagggctttaaaattatttacctagtttagttattatagattcggcagcagcttttagcttagatgcagtgttaatacgatttttaatttcagtatacgcatcgccaagaacatcaaggctatcagaataaacacttacactattgcgttcgctatttgaaatagaacctttagttttacgatcaaaatcagtgtacacgtcttgaagatcacgggctgccttctttgcatcatccaatgcatcaagagctttattcaaagcattgacgtattttttagtgtcaaacatattcttagaaattttaacaggtgcacgttcaggagtagctaaggtggatttaggaacctgtgtgccag

SEQ ID NO: 184 - DNA, 207 bp, synthetic construct
  misc_feature 1..207: note = Type I-E / I-F array
  source 1..207: mol_type = other DNA
gagttccccgcgccagcggggataaaccgctgaagtagaaaaacgtgttacagcatcagtcgagttccccgcgccagcggggataaaccgggagagagtgagcgatcctccgttaacatagttcactgccgtacaggcagcttagaaagtattattcgacggcggtgggattgcttcacgttcactgccgtacaggcagcttagaaa